(19) United States
(12) Patent Application Publication
(10) Pub. No.: US 2025/0388604 A1
(43) Pub. Date: Dec. 25, 2025

(54) Compounds and Their Use as PDE4 Activators

(71) Applicant: Mironid Limited, Glasgow (GB)

(72) Inventors: Julia Mary Adam, Glasgow (GB); David Roger Adams, Glasgow (GB); Rutger Folmer, Nijmegen (NL); Koen Hekking, Nijmegen (NL); Yorik Bruseker, Nijmegen (NL)

(73) Assignee: Mironid Limited, Glasgow (GB)

(21) Appl. No.: 19/103,707

(22) PCT Filed: Aug. 17, 2023
(86) PCT No.: PCT/EP2023/072647

§ 371 Date: Feb. 13, 2025

(30) Foreign Application Priority Data
Aug. 17, 2022 (GB) 2212000.0

(51) Int. Cl. C07D 519/00; A61K 31/444; A61K 31/4545; A61K 31/496; A61K 31/4985; A61K 31/4995; A61P 5/18; A61P 13/12; A61P 35/00; C07D 513/04

(52) U.S. Cl. CPC C07D 519/00; A61K 31/444; A61K 31/4545; A61K 31/496; A61K 31/4985; A61K 31/4995; A61P 5/18; A61P 13/12; A61P 35/00; C07D 513/04

(57) ABSTRACT

The present invention relates to compounds of Formulas I to V and la to Va, their use as activators of long form cyclic nucleotide phosphodiesterase-4 (PDE4) enzymes (isoforms) and to these compounds for use in a method for the treatment or prevention of disorders requiring a reduction of second messenger responses mediated by cyclic 3′,5′-adenosine monophosphate (cAMP).

### FIELD OF THE INVENTION

The present invention relates to compounds as defined herein, their use as activators of long form cyclic nucleotide phosphodiesterase-4 (PDE4) enzymes (isoforms) and to therapies using these compounds. In particular, the invention relates to these compounds for use in a method for the treatment or prevention of disorders requiring a reduction of second messenger responses mediated by cyclic 3′,5′-adenosine monophosphate (cAMP).

### BACKGROUND TO THE INVENTION

Cyclic 3′,5′-adenosine monophosphate—“cAMP”—is a critical intracellular biochemical messenger that is involved in the transduction of the cellular effects of a variety of hormones, neurotransmitters, and other extracellular biological factors in most animal and human cells. The intracellular concentration of cAMP is controlled by the relative balance between its rate of production and degradation. cAMP is generated by biosynthetic enzymes of the adenylyl cyclase superfamily and degraded by members of the cyclic nucleotide phosphodiesterase (PDE) superfamily. Certain members of the PDE superfamily, such as PDE4, specifically degrade cAMP, while others either specifically degrade cyclic guanosine monophosphate (cGMP) or degrade both cAMP and cGMP. PDE4 enzymes inactivate cAMP, thereby terminating its signalling, by hydrolysing cAMP to 5′-AMP (Lugnier, C. Pharmacol Ther. 109: 366-398, 2006).

Four PDE4 genes (PDE4A, PDE4B, PDE4C and PDE4D) have been identified, each of which encodes a number of different enzyme isoforms through the use of alternative promoters and mRNA splicing. On the basis of their primary structures, the catalytically active PDE4 splice variants can be classified as “long”, “short” or “super-short” forms (Houslay, M. D. Prog Nucleic Acid Res Mol Biol. 69: 249-315, 2001). A “dead short” form also exists, which is not catalytically active (Houslay, M. D., Baillie, G. S. and Maurice, D. H. Circ Res. 100: 950-66, 2007). PDE4 long forms have two regulatory regions, called upstream conserved regions 1 and 2 (UCR1 and UCR2), located between their isoform-specific N-terminal portion and the catalytic domain. The UCR1 domain is absent in short forms, whereas the super-short forms not only lack UCR1, but also have a truncated UCR2 domain (Houslay, M. D., Schafer, P. and Zhang, K. Drug Discovery Today 10: 1503-1519, 2005).

PDE4 long forms, but not short forms, associate into dimers within cells (Richter, W and Conti, M. J. Biol. Chem. 277: 40212-40221, 2002; Bolger, G. B. et al., Cell. Signal. 27: 756-769, 2015). A proposed negative allosteric modulation of PDE4 long forms by small molecules has been reported (Burgin A. B. et al., Nat. Biotechnol. 28: 63-70, 2010; Gurney M. E. et al., Handb. Exp. Pharmacol. 204: 167-192, 2011).

It is known in the art that PDE4 long forms may be activated by endogenous cellular mechanisms, such as phosphorylation (Mackenzie, S. J. et al., Br. J. Pharmacol. 136: 421-433, 2002) and phosphatidic acid (Grange et al., J. Biol. Chem. 275: 33379-33387, 2000). Activation of PDE4 long forms by ectopic expression of a 57 amino acid protein (called ‘UCR1C’) whose precise sequence reflects part of that of the upstream conserved region 1 of PDE4D (‘UCR1C’ sequence reflects that of amino acids 80-136 while UCR is amino acids 17-136: numbering based on the PDE4D3 long isoform) has been reported (Wang, L. et al., Cell. Signal. 27: 908-922, 2015: “UCR1C is a novel activator of phosphodiesterase 4 (PDE4) long isoforms and attenuates cardiomyocyte hypertrophy”). The authors hypothesised that PDE4 activation might be used as a potential therapeutic strategy for preventing cardiac hypertrophy.

The first small molecules that act as activators of PDE4 long forms were recently disclosed in WO2016/151300, WO2018/060704 and WO2019/193342. A small molecule activator of PDE4 long forms was recently evaluated in cell-based models of Autosomal Dominant Polycystic Kidney Disease (ADPKD) (Omar et al., PNAS 116: 13320-13329, 2019). No small molecule activators of PDE4 long forms have yet been reported in clinical development. There remains a need for further, structurally distinct small molecule activators of PDE4 long forms for potential development as therapeutic agents.

It is amongst the objects of the present invention to provide new small molecule activators of at least one of the long forms of PDE4 for use in a method of therapy, as well as specific disease treatment or prevention.

### SUMMARY OF THE INVENTION

In a first aspect of the present invention, there is provided a compound of Formula I:

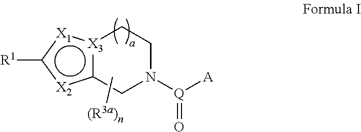

or a pharmaceutically acceptable salt or derivative thereof, wherein:
one of X₁ and X₂ is N and the other is S, N, or CR^{3b}, X₃ is N or C;
Q is C or S(O);
R¹ is a 4- to 10-membered non-aromatic ring system that may be monocyclic, bridged or bicyclic containing at least 1 ring N heteroatom and optionally a ring O heteroatom, and wherein R¹ is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1;
   for use in the treatment or prevention of a disease or disorder that can be ameliorated by activation of long isoforms of PDE4 or a disease or disorder mediated by excessive intracellular cyclic AMP signalling.

In a second aspect of the present invention, there is provided a compound of Formula II:

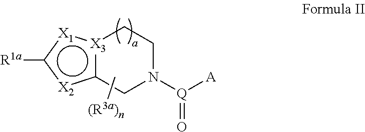

or a pharmaceutically acceptable salt or derivative thereof, wherein:
one of X₁ and X₂ is N and the other is S, N or CR^{3b}, X₃ is N or C; Q is C or S(O);
R^{1a} is a 7- to 9-membered saturated, bridged ring system containing 2 ring N heteroatoms, and wherein R^{1a} is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocyclic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1.

In a third aspect of the present invention, there is provided a compound of Formula III:

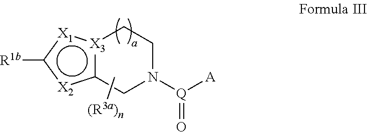

or a pharmaceutically acceptable salt or derivative thereof, wherein:
one of X₁ and X₂ is N and the other is S, N or CR^{3b}, X₃ is N or C;
Q is C or S(O);
R^{1b} is a 4- to 10-membered non-aromatic ring that may monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocyclic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1;
wherein when one of X₁ and X₂ is N and the other is S, X₃ is C, and (a) A is R^{2c} or NR^{2a}R^{2b}, then R^{1b} is not 4-methyl-piperazin-1-yl or 4-ethyl-piperazin-1yl or (b) A is OR^{2f}, then R^{1b} is not a monocyclic piperazine ring.

In a fourth aspect of the present invention, there is provided a Formula IV:

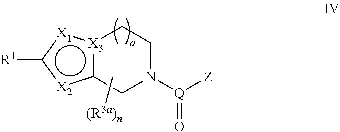

or a pharmaceutically acceptable salt or derivative thereof, wherein:
one of X₁ and X₂ is N and the other is S, N or CR^{3b}, X₃ is N or C; Q is C or S(O);
R¹ is a 4- to 10-membered non-aromatic ring that may be monocyclic, bridged or bicyclic containing at least 1 ring N heteroatom and optionally a ring O heteroatom, and wherein R¹ is optionally substituted with 1 or more R⁴;
Z is R^{2e} or NR^{2d}R^{2b};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1;
optionally wherein the compound is not selected from a group consisting of

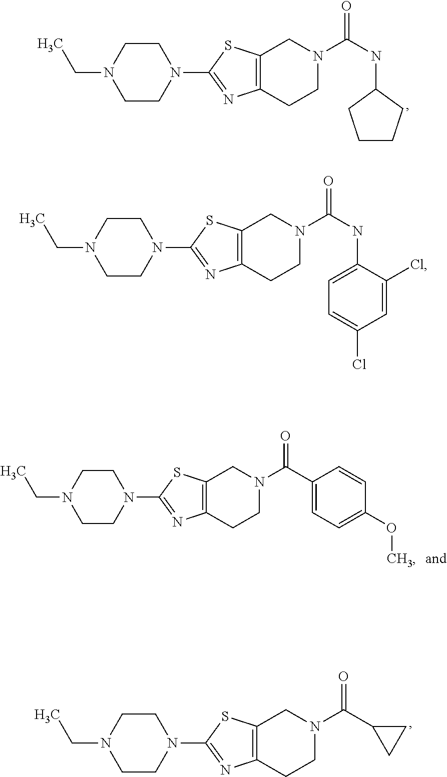 [SMILES: CCN1CCN(c2nc3c(s2)CN(C(=O)NC2CCCC2)CC3)CC1 ; CCN1CCN(c2nc3c(s2)CN(C(=O)Nc2ccc(Cl)cc2Cl)CC3)CC1 ; CCN1CCN(c2nc3c(s2)CN(C(=O)c2ccc(OC)cc2)CC3)CC1 ; "and" ; CCN1CCN(c2nc3c(s2)CN(C(=O)C2CC2)CC3)CC1]

or a pharmaceutically acceptable salt thereof.

In a fifth aspect of the present invention, there is provided a compound of Formula V:

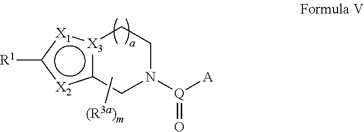

or a pharmaceutically acceptable salt or derivative thereof, wherein:
one of X₁ and X₂ is N and the other is S, N or CR^{3b}, X₃ is N or C;
Q is C or S(O);
R¹ is a 4- to 10-membered non-aromatic ring system that may be monocyclic, bridged or bicyclic containing at least 1 ring N heteroatom and optionally a ring O heteroatom, and wherein R¹ is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
m is 1, 2, 3 or 4; and
a is 0 or 1.

Compounds described herein are shown in the Examples to activate PDE4 long form enzymes.

In a further aspect, the present invention provides a pharmaceutical composition comprising a compound or a pharmaceutically acceptable salt or derivative as described herein, and a pharmaceutically acceptable excipient.

In a further aspect, the present invention provides a compound or pharmaceutical composition described herein for use in therapy. The therapy may be the treatment or prevention of any disease or disorder as described herein. The therapy may be the treatment or prevention of a disease or disorder that can be ameliorated by activation of long isoforms of PDE4. The therapy may be the treatment or prevention of a disease or disorder mediated by excessive intracellular cAMP signalling. In these diseases, a reduction of second messenger responses mediated by cyclic 3′,5′-adenosine monophosphate (cAMP) should provide a therapeutic benefit.

Also provided is a method of treating or preventing a disease or disorder that can be ameliorated by activation of long isoforms of PDE4, comprising the step of administering an effective amount of a compound or pharmaceutical composition described herein to a patient in need thereof. Also provided is a method of treating or preventing a disease or disorder mediated by excessive intracellular cAMP signalling, comprising the step of administering an effective amount of a compound or pharmaceutical composition described herein to a patient in need thereof.

Also provided is the use of a compound or pharmaceutical composition described herein in the manufacture of a medicament for treating or preventing a disease or disorder that can be ameliorated by activation of long isoforms of PDE4. Also provided is the use of a compound or pharmaceutical composition described herein in the manufacture of a medicament for treating or preventing a disease or disorder mediated by excessive intracellular cAMP signalling.

In certain embodiments of the foregoing aspects, the compounds of the invention are provided for the treatment or prevention of cancer. In certain embodiments of the foregoing aspects, the compounds of the invention are provided for the treatment or prevention of a disease or disorder selected from hyperthyroidism, Jansens's metaphyseal chondrodysplasia, hyperparathyroidism, familial male-limited precocious puberty, pituitary adenomas, Cushing's disease, polycystic kidney disease, polycystic liver disease, McCune-Albright syndrome, cholera, whooping cough, anthrax, tuberculosis, HIV, AIDS, Common Variable Immunodeficiency (CVID), melanoma, pancreatic cancer, leukaemia, prostate cancer, adrenocortical tumours, testicular cancer, primary pigmented nodular adrenocortical diseases (PPNAD), Carney Complex, autosomal dominant polycystic kidney disease (ADPKD), autosomal recessive polycystic kidney disease (ARPKD), maturity onset diabetes of young type 5 (MODY5), or cardiac hypertrophy.

### DETAILED DESCRIPTION

The invention is based on the surprising identification of new compounds that are able to activate long isoforms of PDE4 enzymes. The compounds are small molecules and so are expected to be easier and cheaper to make and formulate into pharmaceuticals than large biological molecules such as polypeptides, proteins or antibodies. The compounds can be chemically synthesized, as demonstrated in the Examples.

The Examples demonstrate that a number of compounds of Formula I to V and Ia to Va are able to activate long isoforms of PDE4. The Examples go on to demonstrate that certain tested compounds of the invention do not activate a short form of PDE4, thereby demonstrating selectivity for activation of PDE4 long forms over PDE4 short forms. The Examples further demonstrate that PDE4 long form activators of the present invention reduce cAMP-driven cyst formation in an in vitro model of ADPKD.

Various aspects and embodiments are disclosed herein. It will be recognised that features specified in each embodiment may be combined with other specified features to provide further embodiments.

Described herein are compounds of Formula I to V, or pharmaceutically acceptable salts or derivatives thereof, as set out above. Formula I to V are illustrated herein. Compounds of Formula I to V, or pharmaceutically acceptable salts or derivatives thereof, may be provided for use in the treatment or prevention of a disease or disorder that can be ameliorated by activation of long isoforms of PDE4. Compounds of Formula I to V, or pharmaceutically acceptable salts or derivatives thereof, may be provided for use in the treatment or prevention of a disease or disorder mediated by excessive intracellular cAMP signalling.

Also described herein are compounds of Formula Ia to Va, or pharmaceutically acceptable salts or derivatives thereof, as set out below. Formula Ia to Va are illustrated herein. Compounds of Formula Ia to Va, or pharmaceutically acceptable salts or derivatives thereof, may be provided for use in the treatment or prevention of a disease or disorder that can be ameliorated by activation of long isoforms of PDE4. Compounds of Formula Ia to Va, or pharmaceutically acceptable salts or derivatives thereof, may be provided for use in the treatment or prevention of a disease or disorder mediated by excessive intracellular cAMP signalling.

Described herein is a compound of Formula Ia

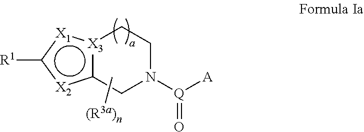

or a pharmaceutically acceptable salt or derivative thereof, wherein:
one of X₁ and X₂ is N and the other is N, S or CR^{3b}, X₃ is N or C; Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Also described herein is a compound of Formula IIa:

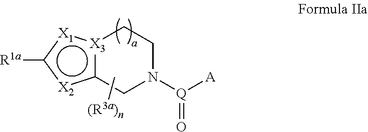

or a pharmaceutically acceptable salt or derivative thereof, wherein:
one of X₁ and X₂ is N and the other is N, S or CR^{3b}, X₃ is N or C;
Q is C or S(O);
R^{1a} is a 7- to 9-membered saturated, bridged ring system containing 2 ring N heteroatoms, and wherein R^{1a} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Also described herein is a compound of Formula IIIa:

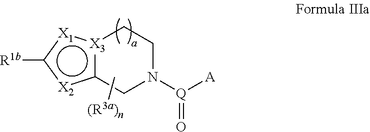

or a pharmaceutically acceptable salt or derivative thereof, wherein:
one of X₁ and X₂ is N and the other is N, S or CR^{3b}, X₃ is N or C;
Q is C or S(O);
R^{1b} is a 4- to 10-membered non-aromatic ring that may monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1;
wherein when one of X₁ and X₂ is N and the other is S and X₃ is C, Rib is not a monocyclic piperazine ring.

Also described herein is a compound of Formula IVa:

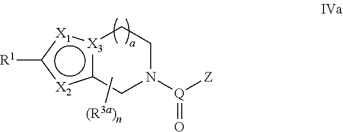

or a pharmaceutically acceptable salt or derivative thereof, wherein:
one of X₁ and X₂ is N and the other is N, S or CR^{3b}, X₃ is N or C;
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.

Also described herein is a compound of Formula Va:

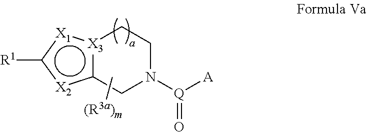

or a pharmaceutically acceptable salt or derivative thereof, wherein:
one of X₁ and X₂ is N and the other is N, S or CR^{3b}, X₃ is N or C;
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
m is 1, 2, 3 or 4; and
a is 0 or 1.

In the compounds of Formula I, R¹ is a 4- to 10-membered non-aromatic ring system that may be monocyclic, bridged or bicyclic, containing at least 1 ring N heteroatom and optionally a ring O heteroatom, and wherein R¹ is optionally substituted with 1 or more R⁴. The monocyclic, bridged or bicyclic ring may be saturated or partially saturated, or in the case of a bicyclic ring, a combination thereof. It will be appreciated that the ring N atom in a saturated or partially saturated ring, when unsubstituted, may be NH (as valency allows). It will also be appreciated that no further ring heteroatoms are present other than the “at least 1 ring N heteroatom” (i.e. 1 or more ring N heteroatoms) and the optional “ring O heteroatom”.

In the compounds of Formula Ia, R¹ is a 4- to 10-membered monocyclic, bridged or bicyclic ring containing at least 1 ring N heteroatom and optionally a ring O heteroatom, and wherein R¹ is optionally substituted with 1 or more R⁴. The monocyclic, bridged or bicyclic ring may be saturated, partially saturated or aromatic, or in the case of a bicyclic ring, a combination thereof. It will be appreciated that the ring N atom in a saturated or partially saturated ring, when unsubstituted, may be NH (as valency allows). It will also be appreciated that no further ring heteroatoms are present other than the “at least 1 ring N heteroatom” (i.e. 1 or more ring N heteroatoms) and the optional “ring O heteroatom”.

In embodiment (1) of Formula I or Ia, R¹ comprises at least 1 ring N heteroatom not at the point of attachment of R¹ (i.e. a ring N atom must be present at a position that not the point of attachment of R¹ to the ring containing X₁, X₂ and X₃). The remaining moieties may be as defined for Formula I or Ia or any of embodiments (6)-(34) of Formula I or Ia described herein, mutatis mutandis.

In embodiment (2) of Formula I or Ia, R¹ is a 4- to 10-membered monocyclic, bridged or bicyclic ring containing 1 ring N heteroatom, 2 ring N heteroatoms or 1 ring N heteroatom and 1 ring O heteroatom, and wherein R¹ is optionally substituted with 1 or more R⁴. R¹ may comprise at least 1 ring N heteroatom not at the point of attachment of R¹. The remaining moieties may be as defined for Formula I or Ia or any of embodiments (6)-(34) of Formula I or Ia described herein, mutatis mutandis.

In embodiment (3) of Formula I, R¹ is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms, optionally 2 ring N heteroatoms; and R¹ is optionally substituted with 1 or more R⁴, optionally wherein R¹ is optionally substituted with 1, 2 or 3 R⁴. R¹ may comprise at least 1 ring N heteroatom not at the point of attachment of R¹. The remaining moieties may be as defined for Formula I or any of embodiments (6)-(34) of Formula I described herein, mutatis mutandis.

In embodiment (3) of Formula Ia, R¹ is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 5- to 6-membered aromatic, monocyclic ring containing 1 or 2 ring N heteroatoms; or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms, optionally 2 ring N heteroatoms; and R¹ is optionally substituted with 1 or more R⁴, optionally wherein R¹ is optionally substituted with 1, 2 or 3 R⁴. R¹ may comprise at least 1 ring N heteroatom not at the point of attachment of R¹. The remaining moieties may be as defined for Formula Ia or any of embodiments (6)-(34) of Formula Ia described herein, mutatis mutandis.

In Formula I or any of the options for embodiments (1), (2) or (3), R¹ may be a 4- to 10-membered monocyclic, bridged or bicyclic ring containing at least 1 ring N heteroatom (i.e. with no ring O heteroatom). R¹ may be a 6-membered saturated monocyclic ring containing 1 or 2 ring N heteroatoms, or a 7- to 8-membered saturated, bridged ring system containing 1 or 2 ring N heteroatoms, and R¹ is optionally substituted with 1 or more R⁴. R¹ may be a 6-membered saturated monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and R¹ is optionally substituted with 1 or more R⁴. R¹ may be a 6-membered saturated monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and wherein R¹ is optionally substituted with 1 R⁴. R¹ may be a 6-membered saturated monocyclic ring containing 2 ring N heteroatoms; or a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, wherein R¹ is optionally substituted with 1 R⁴, R¹ may be a 7- to 8-membered saturated, bridged ring system containing 2 ring N heteroatoms, for example a bridged piperazine, such as 3,8-diazabicyclo[3.2.1]octanyl, wherein R¹ is optionally substituted with 1 R⁴. The remaining moieties may be as defined for Formula I or any of embodiments (6)-(34) of Formula I described herein, mutatis mutandis.

In Formula Ia or any of the options for embodiments (1), (2) or (3), R¹ may be a 4- to 10-membered monocyclic, bridged or bicyclic ring containing at least 1 ring N heteroatom (i.e. with no ring O heteroatom). R¹ may be a 6-membered saturated or aromatic monocyclic ring containing 1 or 2 ring N heteroatoms, or a 7- to 8-membered saturated, bridged ring system containing 1 or 2 ring N heteroatoms, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and wherein R¹ is optionally substituted with 1 R⁴. R¹ may be a 6-membered saturated or aromatic monocyclic ring containing 2 ring N heteroatoms; or a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, wherein R¹ is optionally substituted with 1 R⁴, R¹ may be a 7- to 8-membered saturated, bridged ring system containing 2 ring N heteroatoms, for example a bridged piperazine, such as 3,8-diazabicyclo[3.2.1]octanyl, wherein R¹ is optionally substituted with 1 R⁴. The remaining moieties may be as defined for Formula Ia or any of embodiments (6)-(34) of Formula Ia described herein, mutatis mutandis.

In Formula I or any of the options for embodiments (1), (2) or (3), R¹ may be piperidinyl, piperazinyl, morpholinyl, pyrrolidinyl, azetidinyl, 2,5-diazabicyclo[2.2.1]heptanyl, 2,5-diazabicyclo[2.2.2]octanyl or 3,8-diazabicyclo[3.2.1]octanyl, 3,9-diazabicyclo[3.3.1]nonanyl, 3,6-diazabicyclo[3.1.1]heptanyl, 4,7-diazaspiro[2.5]octanyl, 2,6-diazaspiro[3.3]heptanyl, 2,6-diazaspiro[3.4]octanyl, 2,7-diazaspiro[3.5]nonanyl, octahydro-4H-pyrrolo[3,2-b]pyridinyl, octahydro-5H-pyrrolo[3,2-c]pyridinyl or hexahydropyrrolo[3,4-c]pyrrol-(1H)-yl, each of which is optionally substituted with 1 or more R⁴, preferably optionally substituted with 1-3 R⁴, preferably optionally substituted with 1 R⁴. R¹ may be a group of structure:

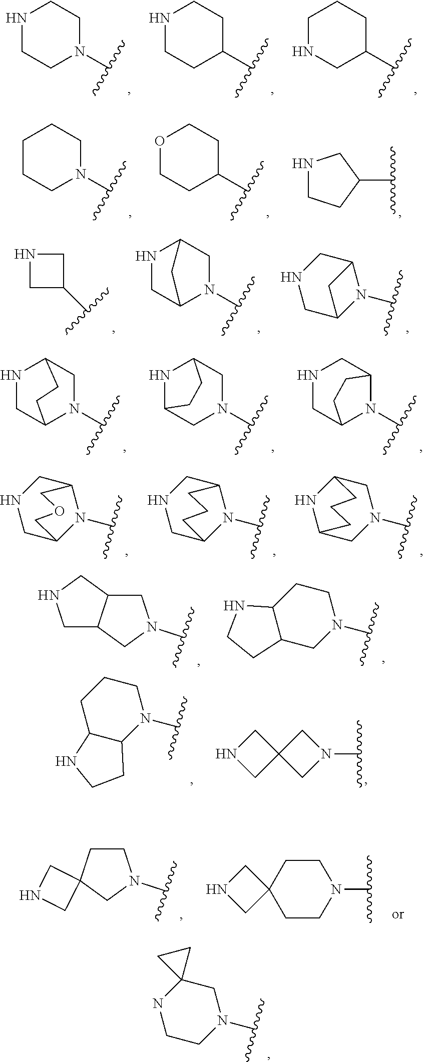

and wherein R¹ is optionally substituted with 1 or more R⁴, optionally wherein R¹ is optionally substituted with 1-3 R⁴. R¹ may be piperidinyl, piperazinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be piperidinyl or piperazinyl, each of which is optionally substituted with 1 or more R⁴, preferably optionally substituted with 1-3 R⁴, preferably optionally substituted with 1 R⁴. R¹ may be: a group of structure

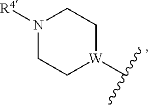

wherein W is CH or N and R^{4′} is H or R⁴. R¹ may be a 7- to 8-membered saturated, bridged ring system containing 2 ring N heteroatoms, for example a bridged piperazine such as

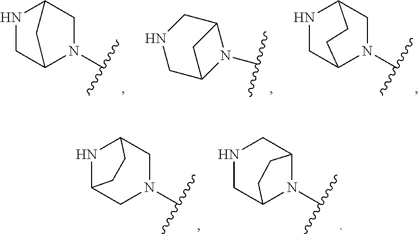 [SMILES: *N1CC2CC1CN2 ; *N1C2CNCC1C2 ; *N1CC2CCC1CN2 ; *N1CC2CCC(C1)N2 ; *N1C2CCC1CNC2]

The remaining moieties may be as defined for Formula I or any of embodiments (6)-(34) of Formula I described herein, mutatis mutandis.

In Formula Ia or any of the options for embodiments (1), (2) or (3), R¹ may be piperidinyl, piperazinyl, morpholinyl, pyrrolidinyl, pyrazolyl, imidazolyl, pyridinyl, azetidinyl, 2,5-diazabicyclo[2.2.1]heptanyl, 2,5-diazabicyclo[2.2.2]octanyl or 3,8-diazabicyclo[3.2.1]octanyl, 3,6-diazabicyclo[3.1.1]heptanyl, 4,7-diazaspiro[2.5]octanyl, 2,6-diazaspiro[3.3]heptanyl, 2,6-diazaspiro[3.4]octanyl, 2,7-diazaspiro[3.5]nonanyl, octahydro-4H-pyrrolo[3,2-b]pyridinyl, octahydro-5H-pyrrolo[3,2-c]pyridinyl or hexahydropyrrolo[3,4-c]pyrrol-(1H)-yl, each of which is optionally substituted with 1 or more R⁴, preferably optionally substituted with 1-3 R⁴, preferably optionally substituted with 1 R⁴. R¹ may be a group of structure:

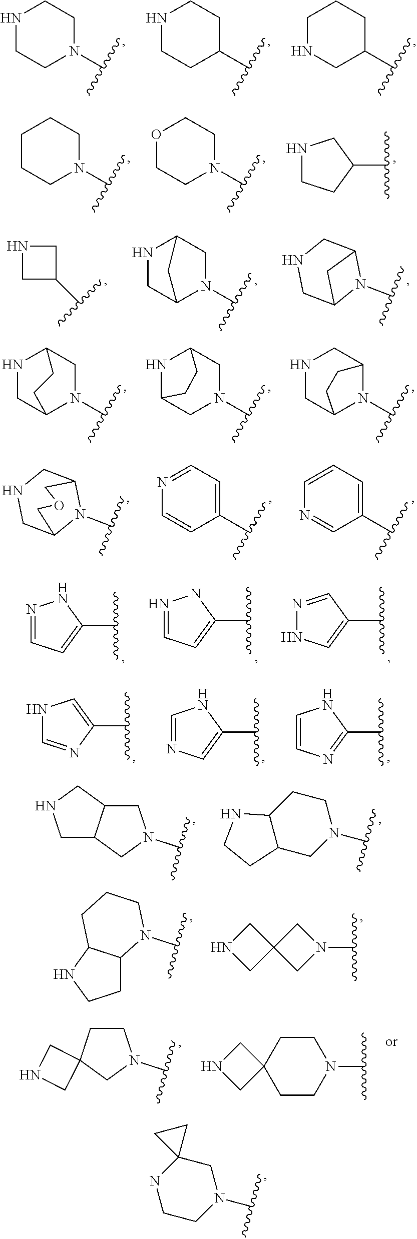

and wherein R¹ is optionally substituted with 1 or more R⁴, optionally wherein R¹ is optionally substituted with 1-3 R⁴. R¹ may be piperidinyl, piperazinyl, pyrrolidinyl, pyrazolyl, imidazolyl, pyridinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be piperidinyl, piperazinyl or pyridinyl, each of which is optionally substituted with 1 or more R⁴, preferably optionally substituted with 1-3 R⁴, preferably optionally substituted with 1 R⁴. R¹ may be: a group of structure

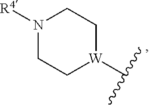

wherein W is CH or N and R^{4′} is H or R⁴; or pyridyl (optionally 3-pyridyl) optionally substituted with 1 R⁴. R¹ may be a 7- to 8-membered saturated, bridged ring system containing 2 ring N heteroatoms, for example a bridged piperazine such as

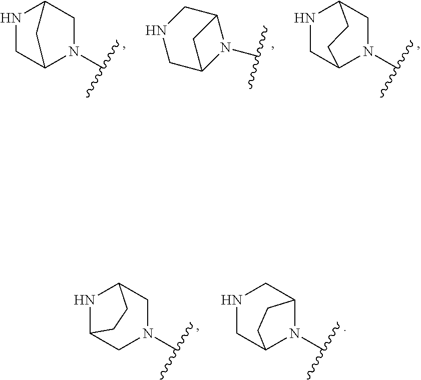 [SMILES: *N1CC2CC1CN2 ; *N1C2CNCC1C2 ; *N1CC2CCC1CN2 ; *N1CC2CCC(C1)N2 ; *N1C2CCC1CNC2]

The remaining moieties may be as defined for Formula Ia or any of embodiments (6)-(34) of Formula Ia described herein, mutatis mutandis.

In embodiment (4) of Formula I or Ia, R¹ is a 5- to 6-membered saturated, monocyclic ring containing at least 1 ring N heteroatom and optionally a ring O heteroatom; or a 7- to 8-membered saturated, bridged ring containing 1 or 2 ring N heteroatoms, and wherein R¹ is optionally substituted with 1, 2 or 3 R⁴. In any of the options for embodiment (4), R¹ may comprise at least 1 ring N heteroatom not at the point of attachment of R¹. R¹ may be a 6-membered saturated monocyclic ring containing 1 or 2 ring N heteroatoms, or a 7- to 8-membered saturated, bridged ring system containing 1 or 2 ring N heteroatoms. R¹ may be a 6-membered saturated monocyclic ring containing 1 or 2 ring N heteroatoms, or a 7- to 8-membered saturated, bridged ring system containing 1 or 2 ring N heteroatoms, wherein R¹ is optionally substituted with 1 R⁴. The remaining moieties may be as defined for Formula I or Ia or any of embodiments (6)-(34) of Formula I or Ia described herein, mutatis mutandis.

In embodiment (5) of Formula I or Ia, R¹ is a 7- to 9-membered saturated, bridged ring system containing 2 ring N heteroatoms, and wherein R¹ is optionally substituted with 1 or more R⁴. R¹ may be a 7- to 9-membered saturated, bridged ring system containing 2 ring N heteroatoms, and wherein R^{1a} is optionally substituted with 1, 2 or 3 R⁴. R¹ may be an optionally substituted 7- to 8-membered saturated, bridged ring system containing 2 ring N heteroatoms. The remaining moieties may be as defined for Formula I or Ia or any of embodiments (6)-(34) of Formula I or Ia described herein, mutatis mutandis.

In Formula I or any of the options for embodiments (1)-(5), R¹ may be substituted with 1 or more R⁴. Where R¹ contains a substitutable ring N atom, R¹ may be substituted on a substitutable ring N atom. In embodiments where R¹ is saturated ring, R¹ may be substituted by 1 R⁴, preferably on a ring N atom. In embodiments where R¹ is a 6-membered ring, R¹ may be substituted by 1 R⁴. In embodiments where R¹ is a 5-membered ring, R¹ may be substituted by 1, 2 or 3 R⁴. R¹ may be substituted by 1 R⁴, preferably on a ring N atom.

In Formula Ia or any of the options for embodiments (1)-(5), R¹ may be substituted with 1 or more R⁴. Where R¹ contains a substitutable ring N atom, R¹ may be substituted on a substitutable ring N atom. In embodiments where R¹ is saturated ring, R¹ may be substituted by 1 R⁴, preferably on a ring N atom. In embodiments where R¹ is an aromatic ring, R¹ may be substituted by 1, 2 or 3 R⁴. In embodiments where R¹ is a 6-membered ring, R¹ may be substituted by 1 R⁴. In embodiments where R¹ is a 5-membered ring, R¹ may be substituted by 1, 2 or 3 R⁴.

In the compounds of Formula I and Ia, each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and (C1-6)alkylene-(C1-6)alkoxy being optionally substituted with 1 or more substituents independently selected from halogen, OH and (C1-6)alkoxy. Each R⁴ may, independently, represent a substituent on a carbon atom or a substitutable N atom.

In embodiment (6) of Formula I or Ia, each R⁴ is independently halogen, OH, CN, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, CN, (C1-4)alkyl, methoxy, ethoxy, cyclopropyl or —(CH₂) 2-O—(CH₂)₂—O—CH₃, the (C1-4)alkyl being optionally substituted with 1 or more substituents independently selected from halogen and OH. The remaining moieties may be as defined for Formula I or Ia or any of embodiments (1)-(5) or (9)-(34) of Formula I or Ia described herein, mutatis mutandis.

In embodiment (7) of Formula I or Ia, each R⁴ is independently halogen, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy. Each R⁴ may independently be halogen, OH, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, (C1-4)alkyl, methoxy, ethoxy, cyclopropyl or —(CH₂)₂—O—(CH₂)₂—O—CH₃, the (C1-4)alkyl being optionally substituted with 1 or more substituents independently selected from halogen and OH. The remaining moieties may be as defined for Formula I or Ia or any of embodiments (1)-(5) or (9)-(34) of Formula I or Ia described herein, mutatis mutandis.

In embodiment (8) of Formula I or Ia, each R⁴ is independently halogen, CN, OH, (C1-3)alkyl, (C1-6)alkoxy, or —(C1-6)alkylene-(C1-6)alkoxy, the (C1-3)alkyl, (C1-6)alkoxy and —(C1-6)alkylene-(C1-6)alkoxy being optionally substituted with 1 or more substituents independently selected from halogen, OH and (C1-6)alkoxy. Each R⁴ may independently be halogen, CN, OH, (C1-2)alkyl, (C1-6)alkoxy, or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy. Each R⁴ may independently be F, Cl, OH, (C1-2)alkyl, methoxy, ethoxy or —(CH₂)₂—O—(CH₂)₂—O—CH₃, the (C1-2)alkyl being optionally substituted with 1 or more substituents independently selected from halogen and OH. The remaining moieties may be as defined for Formula I or Ia or any of embodiments (1)-(5) or (9)-(34) of Formula I or Ia described herein, mutatis mutandis.

In Formula I or Ia or any of the options for embodiments (6)-(8), when attached to a ring N atom, R⁴ may independently be any of the options identified herein for R⁴, except for halogen, CN, OH, and —(C1-6)alkoxy.

In the compounds of Formula I, A is R^{2c}, NR^{2a}R^{2b}, or OR^{2f}, wherein:
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;

In the compounds of Formula Ia, A is NR^{2a}R^{2b} or R^{2c}, wherein
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵.

In the compounds of Formula I, each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy. It will be appreciated that an alkyl group may may be straight chain, branched or cyclic, or a combination thereof.

In the compounds of Formula Ia, each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH. It will be appreciated that an alkyl group may may be straight chain, branched or cyclic, or a combination thereof.

In embodiment (9) of Formula I or Ia, each R⁵ is independently halogen, OH, CN, (C1-4)alkyl, or (C1-4)alkoxy, the (C1-4)alkyl and (C1-4)alkoxy group being optionally substituted with 1 or more halogen or OH, preferably optionally substituted with 1 or more fluoro, 1 OH or 1 (C1-2)alkoxy. Each R⁵ may independently be halogen, OH, CN, (C1-4)alkyl, or (C1-4)alkoxy, the (C1-4)alkyl and (C1-4)alkoxy group being optionally substituted with 1 or more halogen or OH, preferably optionally substituted with 1 or more fluoro or 1 OH. Each R⁵ may independently be halogen, CN or (C1-4)alkyl, the (C1-4)alkyl group being optionally substituted with 1 or more halogen, preferably optionally substituted with 1 or more fluoro. Each R⁵ may independently be halogen (preferably fluoro), CN or CF₃. Each R⁵ may independently be halogen (preferably fluoro). The remaining moieties may be as defined for Formula I or Ia or any of embodiments (1)-(8) or (10)-(34) of Formula I or Ia described herein, mutatis mutandis.

In embodiment (10) of Formula I or Ia, R^{2a} is a 6-membered aromatic or heteroaromatic ring that contains 0, 1 or 2 ring N atoms, wherein R^{2a} is optionally substituted with 1 or more R⁵. R^{2a} may be substituted with 0, 1 or 2 R⁵, preferably 0 or 1 R⁵. R^{2a} may be substituted with halogen, CN or (C1-4)alkyl, the (C1-4)alkyl group being optionally substituted with 1 or more halogen, preferably optionally substituted with 1 or more fluoro. R^{2a} may be substituted with halogen (preferably fluoro), CN or CF₃. The remaining moieties may be as defined for Formula I or Ia or any of embodiments (1)-(9), (14) or (24)-(34) of Formula I or Ia described herein, mutatis mutandis.

In embodiment (11) of Formula I or Ia, R^{2a} is a CH₂-[6-membered aromatic or heteroaromatic ring that contains 0, 1 or 2 ring N atoms], wherein R^{2a} is optionally substituted with 1 or more R⁵. It will be appreciated that substitution by R⁵ is possible on the —CH₂-linker or aromatic or heteroaromatic ring of R^{2a}. R^{2a} is a CH₂-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃. The remaining moieties may be as defined for Formula I or Ia or any of embodiments (1)-(9), (14) or (24)-(34) of Formula I or Ia described herein, mutatis mutandis.

In embodiment (12) of Formula I or Ia, R^{2a} is a (C2-10)alkyl group that may be straight chain, branched or cyclic, or a combination thereof, wherein R^{2a} is optionally substituted with 1 or more R⁵. R^{2a} may be substituted with 0, 1 or 2 R⁵, preferably 0 or 1 R⁵. R^{2a} may be a (C3-10)alkyl group that may be straight chain, branched or cyclic, or a combination thereof, wherein R^{2a} is optionally substituted with R⁵. R^{2a} may be a (C4-10)alkyl group that may be straight chain, branched or cyclic, or a combination thereof, wherein R^{2a} is optionally substituted with R⁵. R^{2a} may be a (C5-10)alkyl group that may be straight chain, branched or cyclic, or a combination thereof, wherein R^{2a} is optionally substituted with R⁵. R^{2a} may be a (C5-10)alkyl group comprising a cyclic moiety, wherein R^{2a} is optionally substituted with R⁵. R^{2c} may be a (C5-8)cycloalkyl group or CH₂—[(C5-6)cycloalkyl group]. R^{2c} may be substituted with 1 or 2 R⁵, R⁵ may preferably substituted by halogen (for example, fluoro). A cyclic moiety in R^{2c} may be substituted with 2 R⁵ (for example, fluoro) at the same carbon atom. The remaining moieties may be as defined for Formula I or Ia or any of embodiments (1)-(9), (14) or (24)-(34) of Formula I or Ia described herein, mutatis mutandis.

In embodiment (13) of Formula I or Ia, R^{2a} is a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵. R^{2a} may be a 6-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵. R^{2a} may be a 5- to 7-membered non-aromatic heterocycle containing one ring O heteroatom fused to a 6-membered aromatic or heteroaromatic ring that contains 0, 1 or 2 ring N atoms; and wherein R^{2a} is optionally substituted with 1 or more R⁵. R^{2a} may be substituted with 0, 1 or 2 R⁵, preferably 0 or 1 R⁵, preferably unsubstituted. The remaining moieties may be as defined for Formula I or Ia or any of embodiments (1)-(9), (14) or (24)-(34) of Formula I or Ia described herein, mutatis mutandis.

In any of embodiments (10)-(13) of Formula I or Ia, R^{2a} may be substituted with 1 or more R⁵. R⁵ may preferably by halogen (for example, fluoro).

In embodiment (14) of Formula I or Ia, R^{2b} is H or (C1-3)alkyl, and wherein (C1-3)alkyl is optionally substituted with 1 or more R⁵. R^{2b} may be H, CH₃ or CH₂CH₃. The remaining moieties may be as defined for Formula I or Ia or any of embodiments (1)-(13) or (24)-(34) of Formula I or Ia described herein, mutatis mutandis.

In embodiment (15) of Formula I or Ia, R^{2a} and R^{2b}, together with the N atom to which they are attached, form a 5- to 10-membered non-aromatic heterocycle, optionally containing 1 further heteroatom selected from O, and optionally substituted with 1 or more R⁵. It will be appreciated that a heterocycle group may be monocyclic or multicyclic (e.g. bicyclic). Multicyclic (e.g. bicyclic) rings may include spiro and fused rings, preferably spiro rings. R^{2a} and R^{2b}, together with the N atom to which they are attached, may form a 5- to 7-membered non-aromatic heterocycle, optionally containing 1 further heteroatom selected from O, and optionally substituted with 1 or more R⁵. R^{2a} and R^{2b}, together with the N atom to which they are attached, may form a 5- to 7-membered non-aromatic heterocycle, optionally substituted with 1 or more R⁵. R^{2a} and R^{2b}, together with the N atom to which they are attached, may form a 5-membered non-aromatic heterocycle, optionally substituted with 1 or more R⁵. A ring formed by R^{2a} and R^{2b} together may be substituted with 0, 1 or 2 (preferably 0 or 1) R⁵. The remaining moieties may be as defined for Formula I or Ia or any of embodiments (1)-(9) or (24)-(34) of Formula I or Ia described herein, mutatis mutandis.

In embodiment (16) of Formula I or Ia, A is R^{2c}. Preferably, Q is also C. The remaining moieties may be as defined for Formula I or Ia or any of embodiments (1)-(9), (17-(22) or (24)-(34) of Formula I or Ia described herein, mutatis mutandis.

In embodiment (17) of Formula I or Ia, R^{2c} is CH₂-[6-membered aromatic or heteroaromatic ring that contains 0, 1 or 2 ring N atoms], wherein R^{2c} is optionally substituted with 1 or more R⁵. It will be appreciated that substitution by R⁵ is possible on the —CH₂-linker or aromatic or heteroaromatic ring of R^{2c}. R^{2c} may be a CH₂-[6-membered aromatic or heteroaromatic ring that contains 0, 1 or 2 ring N atoms], wherein the CH₂ is optionally substituted with 1 or 2 halogen (preferably fluoro, to form —CHF— or —CF₂—), 1 OH or 1 (C1-4)alkoxy and the aromatic or heteroaromatic ring is optionally substituted with 1, 2 or 3 (preferably 1 or 2, preferably 1) halogen, CN, (C1-4)alkyl, or (C1-4)alkoxy the (C1-4)alkyl group being optionally substituted with 1 or more halogen (preferably optionally substituted with 1 or more fluoro). R^{2c} may be a CH₂-[6-membered aromatic or heteroaromatic ring that contains 0, 1 or 2 ring N atoms], wherein the CH₂ is optionally substituted with 1 or 2 halogen (preferably fluoro, to form —CHF— or —CF₂—) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃. The remaining moieties may be as defined for Formula I or Ia or any of embodiments (1)-(9), (16) or (24)-(34) of Formula I or Ia described herein, mutatis mutandis.

In embodiment (18) of Formula I or Ia, R^{2c} is CH₂—O-[6-membered aromatic or heteroaromatic ring that contains 0, 1 or 2 ring N atoms], wherein R^{2c} is optionally substituted with 1 or more R⁵. It will be appreciated that substitution by R⁵ is possible on the —CH₂-linker or aromatic or heteroaromatic ring of R^{2c}. R^{2c} may be a CH₂—O-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN, (C1-4)alkoxy or (C1-4)alkyl, the (C1-4)alkyl group being optionally substituted with 1 or more halogen (preferably optionally substituted with 1 or more fluoro). R^{2c} may be a CH₂—O-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃. The remaining moieties may be as defined for Formula I or Ia or any of embodiments (1)-(9), (14) or (24)-(34) of Formula I or Ia described herein, mutatis mutandis.

In embodiment (19) of Formula I or Ia, R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; wherein R^{2c} is optionally substituted with 1 or more R⁵. It will be appreciated that a cyclic moiety includes multicyclic (e.g. bicyclic) moieties, for example, fused, bridged or spiro bicyclic rings. R^{2c} may be a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms, and wherein R^{2c} is optionally substituted with 1 or more R⁵. R^{2c} may be a (C3-10)alkyl group comprising a monocyclic moeity, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms, and wherein R^{2c} is optionally substituted with 1 or more R⁵. R^{2c} may be a (C5-10)alkyl group comprising a cyclic moeity; and wherein R^{2c} is optionally substituted with 1 or more R⁵. R^{2c} may be a (C5-6)cycloalkyl group or (C1-2)alkyl-[(C4-6)cycloalkyl group]. R^{2c} may be substituted with 1 or 2 R⁵ and R⁵ may preferably by halogen (for example, fluoro). R^{2c} may be substituted with 1 or more substituents selected from halogen, OH and CN. R^{2c} may be substituted with 1, 2 or 3 halogen, 1 OH or 1 CN. A cyclic moiety in R^{2c} may be substituted with 2 R⁵ (for example, fluoro) at the same carbon atom. The remaining moieties may be as defined for Formula I or Ia or any of (1)-(9), (16) or (24)-(34) of Formula I or Ia described herein, mutatis mutandis.

In embodiment (20) of Formula I or Ia, R^{2c} is a (C3-10)alkyl group that may be straight chain or branched, wherein a C atom of said (C3-10)alkyl group may be optionally replaced by 1 —O— other than at the point of attachment of R^{2c}, wherein said (C3-10)alkyl group may optionally be substituted with a 6-membered aromatic or heteroaromatic ring that contains 0, 1 or 2 ring N atoms; wherein R^{2c} is optionally substituted with 1 or more R⁵. R^{2c} may be a (C3-10)alkyl group that may be straight chain or branched, wherein a C atom of said (C3-10)alkyl group may be optionally replaced by 1 —O— other than at the point of attachment of R^{2c}; wherein R^{2c} is optionally substituted with 1 or more R⁵. R^{2c} may be a (C3-10)alkyl group that may be straight chain or branched, wherein R^{2c} is optionally substituted with 1 or more R⁵. R^{2c} may be substituted with 1 or more substituents selected from halogen, OH and CN. R^{2c} may be unsubstituted or substituted with 1 substituent. The remaining moieties may be as defined for Formula I or Ia or any of (1)-(9), (16) or (24)-(34) of Formula I or Ia described herein, mutatis mutandis.

In any of embodiments (17)-(20) of Formula I or Ia, R^{2c} may be substituted with 1 or more R⁵. R⁵ may be halogen, for example fluoro.

In embodiment (21) of Formula I or Ia, R^{2c} is a 6-membered aromatic or heteroaromatic ring that contains 1 or 2 ring N atoms. R^{2c} may be phenyl or pyridyl. R^{2c} may be optionally substituted by 1, 2 or 3 (preferably 1 or 2, preferably 1) halogen, CN, (C1-4)alkoxy or (C1-6)alkyl, the (C1-6)alkyl or (C1-4)alkoxy group being optionally substituted with 1 or more halogen (preferably optionally substituted with 1 or more fluoro) and the (C1-6)alkyl group being optionally substituted with (C1-4)alkoxy. It will be appreciated that alkyl group may be straight chain, branched or cyclic or a combination thereof. The remaining moieties may be as defined for Formula I or Ia or any of (1)-(9), (15) or (19)-(26) of Formula I or Ia described herein, mutatis mutandis.

In embodiment (22) of Formula I or Ia, R^{2c} is a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms. It will be appreciated that R^{2c} may be attached to the rest of the compound through either the 5- to 6-membered aromatic or non-aromatic heterocycle or the 6-membered aromatic or heteroaromatic ring. R^{2c} may be a 9-membered bicyclic ring system comprising a 5-membered aromatic heterocycle containing 1, 2 or 3 ring N heteroatoms and/or 1 O heteroatom fused to a 6-membered aromatic or heteroaromatic ring that contains 0 or 1 ring N atoms; a 9- or 10-membered bicyclic ring system comprising a 5- to 6-membered non-aromatic heterocycle containing 1 or 2 ring O heteroatoms fused to a 6-membered aromatic or heteroaromatic ring that contains 0 or 1 ring N atoms; a 10-membered bicyclic ring system comprising a 6-membered aromatic or non-aromatic heterocycle containing 1 or 2 ring N heteroatoms fused to a 6-membered aromatic or heteroaromatic ring that contains 0, 1 or 2 ring N atoms. R^{2c} may be benzofuranyl, 2,3-dihydro-1-benzofuranyl, 1,3-benzoxazolyl, 1,3,3a-triazaindenyl, 1,3-benzodioxolyl, indolyl, quinolyl, isoquinolyl, chromanyl, isochromanyl or 2,3-dihydro-1,4-benzodioxinyl. The remaining moieties may be as defined for Formula I or Ia or any of (1)-(9), (15) or (19)-(26) of Formula I or Ia described herein, mutatis mutandis.

In embodiment (23) of Formula I, R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵. It will be appreciated that substitution by R⁵ is possible on the —CH₂-linker or aromatic or heteroaromatic ring of R^{2f}. R^{2f} may be CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵. The remaining moieties may be as defined for Formula I or any of embodiments (1)-(9) or (21)-(31) of Formula I described herein, mutatis mutandis.

In the compounds of Formula I and Ia, each R^{3a} is independently (C1-6)alkyl or fluoro, the (C1-6)alkyl being optionally substituted by 1 or more halogen; or two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen; and each R^{3b} is independently H or (C1-6)alkyl.

In embodiment (24) of Formula I and Ia, each R^{3a} is independently (C1-3)alkyl or fluoro, the (C1-3)alkyl being optionally substituted by 1 or more halogen and/or each R^{3b} is independently H or (C1-3)alkyl. Each R^{3a} may be —CH₃ or F. Each R^{3b} may be —CH₃ or H. The remaining moieties may be as defined for Formula I or Ia or any of embodiments (1)-(18) or (21)-(26) of Formula I or Ia described herein, mutatis mutandis.

In embodiment (25) of Formula I and Ia, two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen. Two R^{3a} attached to the same carbon may be joined together with the atom to which they are attached to form a 3- to 6-membered cycloalkyl ring, or 5- to 6-membered heterocycloalky ring containing an O heteroatom. Two R^{3a} attached to the same or adjacent carbon atoms may be joined together with the atoms to which they are attached to form a 3- to 6-membered ring, wherein said ring is optionally substituted by 1 or more halogen. Two R^{3a} attached to the same or adjacent carbon atoms may be joined together with the atoms to which they are attached to form a 3- to 6-membered ring. It will be appreciated that when two R^{3a} are attached to the same carbon atom, a spiro ring will be formed and when two R^{3a} are attached to adjacent carbon atoms, a fused ring will be formed. Two R^{3a} attached to the same carbon may be joined together with the atom to which they are attached to form a cyclopropyl ring. The remaining moieties may be as defined for Formula I or Ia or any of embodiments (1)-(18) or (21)-(26) of Formula I or Ia described herein, mutatis mutandis.

In embodiment (26) of Formula I or Ia, each R^{3b} may independently be H or (C1-3)alkyl. Each R^{3b} may be —CH₃ or H. The remaining moieties may be as defined for Formula I or Ia or any of embodiments (1)-(18) or (21)-(26) of Formula I or Ia described herein, mutatis mutandis.

In the compounds of Formula I and Ia, n is 0, 1, 2, 3 or 4.

In embodiment (27) of Formula I and Ia, n is 0. The remaining moieties may be as defined for Formula I or Ia or any of embodiments (1)-(18) or (21)-(26) of Formula I or Ia described herein, mutatis mutandis.

In embodiment (28) of Formula I and Ia, n is 0, 1, 2 or 3. Preferably, n is 0, 1 or 2. n may be 1 or 2. Where n is 2, both R^{3a} are preferably on the same ring carbon atom. The remaining moieties may be as defined for Formula I or Ia or any of embodiments (1)-(20) or (22)-(26) of Formula I or Ia described herein, mutatis mutandis.

In the compounds of Formula I and Ia, Q is C or S(O).

In embodiment (29) of Formula I or Ia, Q is C. The remaining moieties may be as defined for Formula I or Ia or any of embodiments (1)-(21) or (23)-(26) of Formula I or Ia described herein, mutatis mutandis.

In embodiment (30) of Formula I or Ia, Q is S═O and A is R^{2c}. R^{2c} may be a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; or 6-membered aromatic or heteroaromatic ring that contains 0, 1 or 2 ring N atoms. R^{2c} may be according to embodiment (17), (19) or (20). The remaining moieties may be as defined for Formula I or Ia or any of embodiments (1)-(9), (19)-(21) or (23)-(26) of Formula I or Ia described herein, mutatis mutandis.

In the compounds of Formula I or Ia, one of X₁ and X₂ is N and the other is N, S or CR^{3b}, X₃ is N or C.

In embodiment (31), the compound of Formula I or Ia may be a compound of structure:

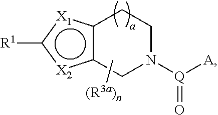

or a pharmaceutically acceptable salt or derivative thereof, wherein one of X₁ and X₂ is N and the other is S. The compound of Formula I or Ia may be a compound of structure:

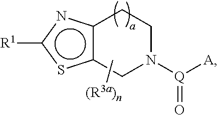

or a pharmaceutically acceptable salt or derivative thereof. The remaining moieties are as defined for Formula I or Ia or any of embodiments (1)-(22) or (25)-(27) of Formula I or Ia described herein, mutatis mutandis.

In embodiment (32), the compound of Formula I or Ia may be a compound of structure:

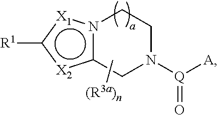

or a pharmaceutically acceptable salt or derivative thereof, wherein one X₁ and X₂ is N and the other is CR^{3b} or both of X₁ and X₂ are N. The compound of Formula I or Ia may be a compound of structure:

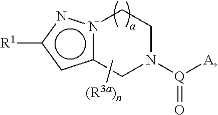

or a pharmaceutically acceptable salt or derivative thereof. The remaining moieties are as defined for Formula I or Ia or any of embodiments (1)-(22) or (25)-(27) of Formula I or Ia described herein, mutatis mutandis.

In the compounds of Formula I and Ia, a is 0 or 1.

In embodiment (33) of Formula I or Ia, a is 0. The compound of Formula I or Ia may be:

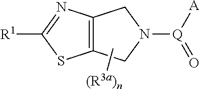

or a pharmaceutically acceptable salt or derivative thereof. The remaining moieties may be as defined for Formula I or Ia or any of embodiments (1)-(24) or (27) of Formula I or Ia described herein, mutatis mutandis.

In embodiment (34) of Formula I or Ia, a is 1. The compound of Formula I or Ia may be:

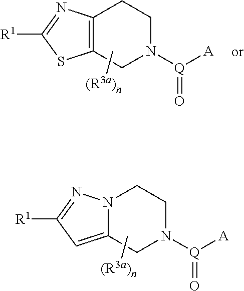

or a pharmaceutically acceptable salt or derivative thereof. The remaining moieties may be as defined for Formula I or Ia or any of embodiments (1)-(24) or (27) of Formula I or Ia described herein, mutatis mutandis.

In embodiment (35) of Formula I or Ia:
Q is C;
R¹ is a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2. The remaining moieties may be as defined for Formula I or Ia or any of embodiments (1)-(34) of Formula I or Ia described herein, mutatis mutandis. For example, preferably X₃ is C and one of X₁ and X₂ is S and the other is N (preferably X₁ is N and X₂ is S). n may be 0. R^{2c} may be as defined in embodiment (19) or (20) of Formula I or Ia.

Compounds of Formula I include compounds of Formulas II to V. Compounds of Formula Ia include compounds of Formulas IIa to Va. Embodiments (1)-(35) of Formula I or Ia may apply mutatis mutandis to each of Formulas II to V or IIa to Va.

Described herein is a compound of Formula II:

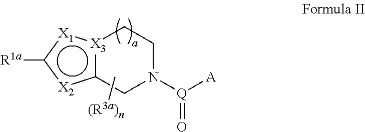

or a pharmaceutically acceptable salt or derivative thereof, wherein:
R^{1a} is a 7- to 9-membered saturated, bridged ring system containing 2 ring N heteroatoms, and wherein R^{1a} is optionally substituted with 1 or more R⁴;
and X₁, X₂, X₃, R^{2a}, R^{2b}, R^{2c}, R^{2f}, R^{3a}, R^{3b}, A, Q, R⁴, R⁵, a and n are as defined for Formula I or any of embodiments (6)-(34) of Formula I above.

Also described herein is a compound of Formula IIa:

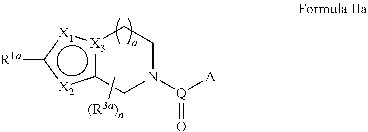

or a pharmaceutically acceptable salt or derivative thereof, wherein:
R^{1a} is a 7- to 9-membered saturated, bridged ring system containing 2 ring N heteroatoms, and wherein R^{1a} is optionally substituted with 1 or more R⁴;
and X₁, X₂, X₃, R^{2a}, R^{2b}, R^{2c}, R^{3a}, R^{3b}, A, Q, R⁴, R⁵, a and n are as defined for Formula Ia or any of embodiments (6)-(34) of Formula Ia above.

In embodiment (1) of Formula II or IIa, R^{1a} is a 7- to 8-membered saturated, bridged ring system containing 2 ring N heteroatoms, wherein R¹ is optionally substituted with 1 or more R⁴, e.g. optionally substituted with 1, 2 or 3 R⁴. R^{1a} may be a 7- to 8-membered saturated, bridged ring system containing 2 ring N heteroatoms, wherein R¹ is optionally substituted with 1 R⁴. R^{1a} may be a bridged piperazine, such as 3,8-diazabicyclo[3.2.1]octanyl, wherein R^{1a} is optionally substituted with 1 R⁴. R^{1a} may be a bridged piperazine such as

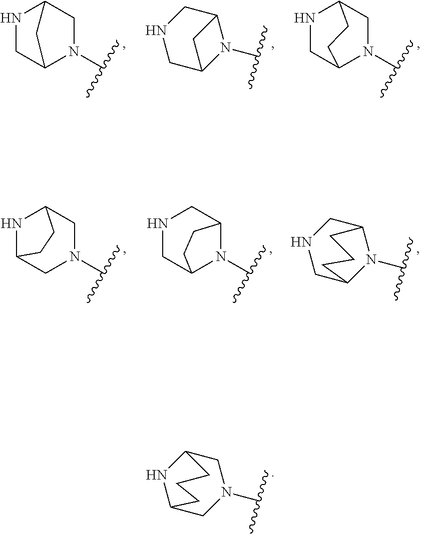

In Formula II or IIa or any of the embodiments of Formula II or IIa, R^{1a} may be optionally substituted with 1 or more R⁴. Where R^{1a} contains a substitutable ring N atom, R^{1a} may preferably be unsubstituted or substituted on a substitutable ring N atom. R^{1a} may be unsubstituted or substituted by 1 R⁴, preferably on a ring N atom.

In Formula II or IIa or any of the embodiments of Formula II or IIa, A may be R^{2c} or NR^{2a}R^{2b}.

In embodiment (2) of Formula II or IIa:
Q is C;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵ R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

In embodiment (2) of Formula II or IIa, preferably X₃ is C and one of X₁ and X₂ is S and the other is N (preferably X₁ is N and X₂ is S), n may be 0, R^{2c} may be as defined in embodiment (19) or (20) of Formula I or Ia.

Described herein is a compound of Formula III:

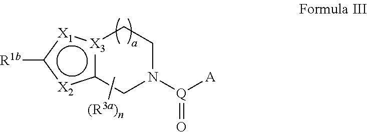

or a pharmaceutically acceptable salt or derivative thereof, wherein:
R^{1b} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
and X₁, X₂, X₃, R^{2a}, R^{2b}, R^{2c}, R^{2f}, R^{3a}, R^{3b}, A, Q, R⁴, R⁵, a and n are as defined for Formula I or any of embodiments (6)-(34) of Formula I above.

Also described herein is a compound of Formula IIIa:

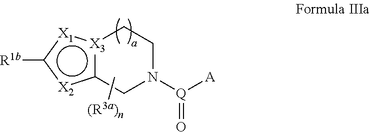

or a pharmaceutically acceptable salt or derivative thereof, wherein:
R^{1b} is a 4- to 10-membered non-aromatic ring that may monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
and X₁, X₂, X₃, R^{2a}, R^{2b}, R^{2c}, R^{3a}, R^{3b}, A, Q, R⁴, R⁵, a and n are as defined for Formula Ia or any of embodiments (6)-(34) of Formula Ia above.

In a compound of Formula III or IIIa, R^{1b} comprises at least one ring N heteroatom not at the point of attachment to R^{1b}, i.e. a ring N atom must be present at a position that is not the point of attachment of R^{1b} to the ring containing X₁, X₂, and X₃.

In embodiment (1) of Formula III or IIIa, R^{1b} is a 4- to 10-membered non-aromatic, monocyclic, bridged or bicyclic ring containing 1 ring N heteroatom, 2 ring N heteroatoms or 1 ring N heteroatom and 1 ring O heteroatom, and wherein R¹ is optionally substituted with 1 or more R⁴. R^{1b} may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 7- to 8-membered saturated, bridged ring system containing 1 or 2 ring N heteroatoms; or a 9-membered saturated, bridged ring system containing 2 ring N heteroatoms and a ring O-heteroatom; or a 7- to 10-membered saturated, fused or spiro ring system containing 1 or 2 ring N heteroatoms, and wherein R^{1b} is optionally substituted with 1 or more R⁴, optionally 1, 2 or 3 R⁴.

In embodiment (2) of Formula III or IIIa, R^{1b} is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); or a 7- to 8-membered saturated, bridged ring containing 1 or 2 ring N heteroatoms, and wherein R^{1b} is optionally substituted with 1 or more R⁴, optionally 1, 2 or 3 R⁴. R^{1b} may be a 6-membered saturated monocyclic ring containing 1 or 2 ring N heteroatoms, optionally wherein at least 1 ring N heteroatom is not at the point of attachment of R^{1b}. R^{1b} may be a 6-membered saturated monocyclic ring containing 1 or 2 ring N heteroatoms, wherein R^{1b} is optionally substituted with 1 R⁴. R^{1b} may be a 7- to 8-membered saturated, bridged ring system containing 1 or 2 ring N heteroatoms, and wherein R^{1b} is optionally substituted with 1 or more R⁴, optionally 1, 2 or 3 R⁴. R^{1b} may be a 7- to 8-membered saturated, bridged ring system containing 2 ring N heteroatoms, for example a bridged piperazine, such as 3,8-diazabicyclo[3.2.1]octanyl, wherein R^{1b} is optionally substituted with 1 R⁴.

In Formula III or IIIa or any of the embodiments of Formula III or IIIa, R^{1b} may be piperidinyl, piperazinyl, morpholinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1b} may be group of structure:

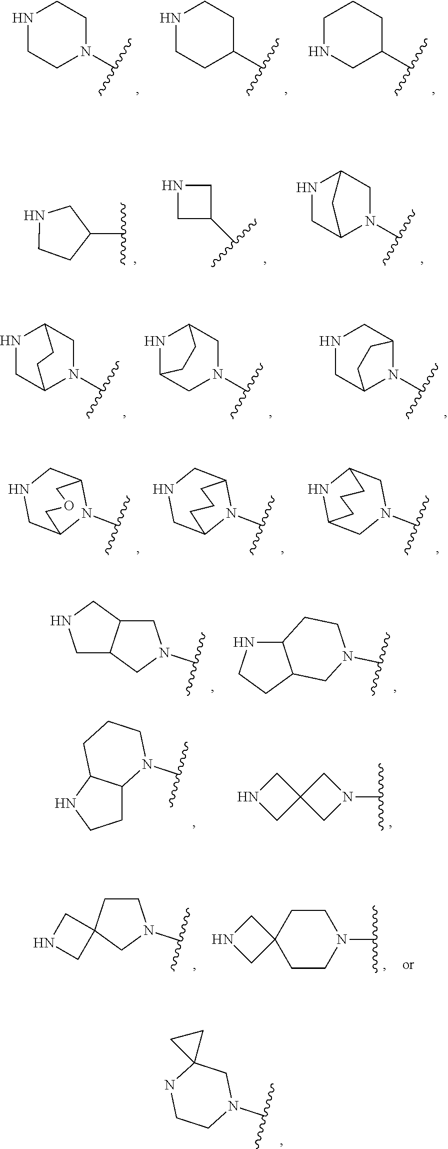

and wherein R^{1b} is optionally substituted with 1 or more R⁴, optionally wherein R^{1b} is optionally substituted with 1-3 R⁴, preferably optionally substituted with 1 R⁴. R^{1b} may be piperidinyl, piperazinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1b} may be piperidinyl or piperazinyl, each of which is optionally substituted with 1 or more R⁴, preferably optionally substituted with 1-3 R⁴, preferably optionally substituted with 1 R⁴. R^{1b} may be a group of structure

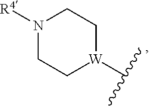

wherein W is CH or N and R^{4′} is H or R⁴. R^{1b} may be a 7- to 8-membered saturated, bridged ring system containing 2 ring N heteroatoms, for example a bridged piperazine such as

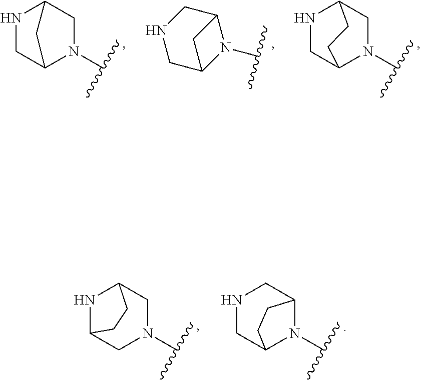 [SMILES: *N1CC2CC1CN2 ; *N1C2CNCC1C2 ; *N1CC2CCC1CN2 ; *N1CC2CCC(C1)N2 ; *N1C2CCC1CNC2]

In Formula III or IIIa or any of the embodiments of Formula III or IIIa, R^{1b} may be optionally substituted with 1 or more R⁴. Where R^{1b} contains a substitutable ring N atom R^{1b} may preferably be substituted on a substitutable ring N atom. R^{1b} may be substituted by 1 R⁴, preferably on a ring N atom.

In Formula III or IIIa or any of the embodiments of Formula III or IIIa, R^{1b} may be a 4- to 10-membered non-aromatic, monocyclic, bridged or bicyclic ring containing at least 1 ring N heteroatom (i.e. with no ring O heteroatom). R^{1b} may be a 6-membered saturated monocyclic ring containing 1 or 2 ring N heteroatoms, or a 7- to 8-membered saturated, bridged ring system containing 1 or 2 ring N heteroatoms, and R^{1b} is optionally substituted with 1 or more R⁴.

In Formula III or IIIa or any of the embodiments of Formula III or IIIa, A may be R^{2c} or NR^{2a}R^{2b}.

In Formula III or IIIa or any of the embodiments for Formula III or IIIa, optionally when one of X₁ and X₂ is N and the other is S, X₃ is C, and A is R^{2c}, then R^{1b} is not 4-methyl-piperazin-1-yl or 4-ethyl-piperazin-1yl.

In Formula III or IIIa or any of the embodiments for Formula III or IIIa, optionally when one of X₁ and X₂ is N and the other is S, X₃ is C and A is OR^{2f}, then R^{1b} is not a (substituted or unsubstituted) monocyclic piperazine ring.

In Formula III or IIIa or any of the embodiments for Formula III or IIIa, optionally when one of X₁ and X₂ is N and the other is S, and X₃ is C, then R^{1b} is not a (substituted or unsubstituted) monocyclic piperazine ring.

In embodiment (3) of Formula III or IIIa:
Q is C;
R^{1b} is a 7- to 9-membered saturated, bridged ring system containing 2 ring N heteroatoms, wherein R^{1b} is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵ R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

In embodiment (3) of Formula III or IIIa, preferably X₃ is C and one of X₁ and X₂ is S and the other is N (preferably X₁ is N and X₂ is S), n may be 0, R^{2c} may be as defined in embodiment (19) or (20) of Formula I or Ia.

Described herein is a compound of Formula IV:

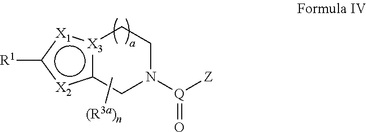

or a pharmaceutically acceptable salt or derivative thereof, wherein:
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; or 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2e} is substituted with 1 or more R⁵ (optionally wherein R⁵ is halogen);
   each R^{3a} is independently (C1-6)alkyl, optionally substituted by 1 or more halogen; or two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen.
and X₁, X₂, X₃, R¹, R^{3a}, R^{3b}, Q, R⁴, R⁵, a and n are as defined for Formula I or any of embodiments (1)-(9) and (24)-(34) of Formula I above.

Also described herein is a compound of Formula IVa:

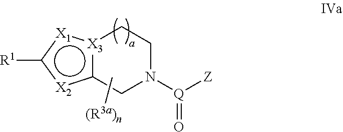

or a pharmaceutically acceptable salt or derivative thereof, wherein:
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
   each R^{3a} is independently (C1-6)alkyl, optionally substituted by 1 or more halogen; or two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen.
and X₁, X₂, X₃, R¹, R^{3a}, R^{3b}, Q, R⁴, R⁵, a and n are as defined for Formula Ia or any of embodiments (1)-(9) and (24)-(34) of Formula Ia above.

In embodiment (1) of Formula IV or IVa, R^{2d} is a 6-membered aromatic or heteroaromatic ring that contains 0, 1 or 2 ring N atoms, wherein R^{2d} is substituted with 1 or more R⁵. R^{2d} may be substituted with 1 or 2 R⁵, preferably 1 R⁵. R^{2d} may be substituted with halogen, CN or (C1-4)alkyl, the (C1-4)alkyl group being optionally substituted with 1 or more halogen, preferably optionally substituted with 1 or more fluoro. R^{2d} may be substituted with halogen (preferably fluoro), CN or CF₃.

In embodiment (2) of Formula IV or IVa, R^{2d} is a CH₂-[6-membered aromatic or heteroaromatic ring that contains 0, 1 or 2 ring N atoms], wherein R^{2d} is substituted with 1 or more R⁵. It will be appreciated that substitution by R⁵ is possible on the —CH₂-linker or aromatic or heteroaromatic ring of R^{2d}. R^{2d} is a CH₂-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃.

In embodiment (3) of Formula IV or IVa, R^{2d} is a (C2-10)alkyl group that may be straight chain, branched or cyclic, or a combination thereof, wherein R^{2d} is substituted with 1 or more R⁵. R^{2d} may be substituted with 1 or 2 R⁵, preferably 1 R⁵. R^{2d} may be a (C3-10)alkyl group that may be straight chain, branched or cyclic, or a combination thereof, wherein R^{2d} is substituted with R⁵. R^{2d} may be a (C4-10)alkyl group that may be straight chain, branched or cyclic, or a combination thereof, wherein R^{2d} is substituted with R⁵. R^{2d} may be a (C5-10)alkyl group that may be straight chain, branched or cyclic, or a combination thereof, wherein R^{2d} is substituted with R⁵. R^{2d} may be a (C5-10)alkyl group comprising a cyclic moiety, wherein R^{2d} is substituted with R⁵. The remaining moieties may be as defined for Formula Ia or any of embodiments (1)-(9), (13) or (19)-(26) of Formula Ia described herein, mutatis mutandis.

In embodiment (4) of Formula IV or IVa, R^{2d} is a (C5-10)alkyl group comprising a cyclic moeity; and wherein R^{2d} is optionally substituted with 1 or more R⁵, R^{2d} may be a (C5-8)cycloalkyl group or CH₂—[(C5-6)cycloalkyl group]. R^{2d} may be substituted with 1 or 2 R⁵, R⁵ may preferably by halogen (for example, fluoro). A cyclic moiety in R^{2d} may be substituted with 2 R⁵ (for example, fluoro) at the same carbon atom.

In embodiment (5) of Formula IV or IVa, R^{2b} is H or (C1-3)alkyl, and wherein (C1-3)alkyl is optionally substituted with 1 or more R⁵. R^{2b} may be H, CH₃ or CH₂CH₃.

In embodiment (6) of Formula IV or IVa, Z is R^{2e}. Preferably, Q is also C.

In embodiment (7) of Formula IV or IVa, R^{2e} is CH₂-[6-membered aromatic or heteroaromatic ring that contains 0, 1 or 2 ring N atoms], wherein R^{2e} is substituted with 1 or more R⁵. It will be appreciated that substitution by R⁵ is possible on the —CH₂-linker or aromatic or heteroaromatic ring of R^{2e}. R^{2e} may be a CH₂-[6-membered aromatic or heteroaromatic ring that contains 0, 1 or 2 ring N atoms], wherein the CH₂ is optionally substituted with 1 or 2 halogen (preferably fluoro), 1 OH or 1 (C1-4)alkoxy and the aromatic or heteroaromatic ring is optionally substituted with 1, 2 or 3 (preferably 1 or 2, preferably 1) halogen, CN, (C-4)alkoxy, or (C1-4)alkyl, the (C1-4)alkyl group being optionally substituted with 1 or more halogen (preferably optionally substituted with 1 or more fluoro). R^{2e} may be a CH₂-[6-membered aromatic or heteroaromatic ring that contains 0, 1 or 2 ring N atoms], wherein the CH₂ is optionally substituted with 1 or 2 halogen (preferably fluoro, to form —CHF— or —CF₂—) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃.

In embodiment (8) of Formula IV or IVa, R^{2e} is CH₂—O-[6-membered aromatic or heteroaromatic ring that contains 0, 1 or 2 ring N atoms], wherein R^{2e} is substituted with 1 or more R⁵. It will be appreciated that substitution by R⁵ is possible on the —CH₂-linker or aromatic or heteroaromatic ring of R^{2e}. R^{2e} may be a CH₂—O-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN, (C-4)alkoxy or (C1-4)alkyl, the (C1-4)alkyl group being optionally substituted with 1 or more halogen (preferably optionally substituted with 1 or more fluoro). R^{2e} may be a CH₂—O-[6-membered aromatic or heteroaromatic ring that contains 0, 1 or 2 ring N atoms], wherein the CH₂ is optionally substituted with 1 or 2 halogen (preferably fluoro) and the aromatic or heteroaromatic ring is optionally substituted with 1, 2 or 3 (preferably 1 or 2, preferably 1) halogen, CN or (C1-4)alkyl, the (C1-4)alkyl group being optionally substituted with 1 or more halogen (preferably optionally substituted with 1 or more fluoro). The aromatic or heteroaromatic ring may be optionally substituted with halogen (preferably fluoro), CN or CF₃.

In embodiment (9) of Formula IV or IVa, R^{2e} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; wherein R^{2e} is substituted with 1 or more R⁵. It will be appreciated that a cyclic moiety includes multicyclic (e.g. bicyclic) moieties, for example, fused, bridged or spiro bicyclic rings. R^{2e} may be a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms. and wherein R^{2e} is substituted with 1 or more R⁵. R^{2e} may be a (C3-10)alkyl group comprising a monocyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms, and wherein R^{2e} is substituted with 1 or more R⁵. R^{2e} may be substituted with 1 or more substituents selected from halogen, OH and CN. R^{2e} may be substituted with 1, 2 or 3 halogen, 1 OH or 1 CN.

In embodiment (10) of Formula IV or IVa, R^{2e} is a (C5-10)alkyl group comprising a cyclic moeity; and wherein R^{2e} is optionally substituted with 1 or more R⁵. It will be appreciated that a cyclic moiety includes multicyclic (e.g. bicyclic) moieties, for example, fused, bridged or spiro bicyclic rings. R^{2e} may be a (C5-10)alkyl group comprising a monocyclic moiety, and wherein R^{2e} is optionally substituted with 1 or more R⁵. R^{2e} may be a (C5-6)cycloalkyl group or (C1-2)alkyl-[(C4-6)cycloalkyl group]. R^{2e} may be substituted with 1 or 2 R⁵, R⁵ may preferably by halogen (for example, fluoro). A cyclic moiety in R^{2e} may be substituted with 2 R⁵ (for example, fluoro) at the same carbon atom.

In embodiment (11) of Formula IV or IVa, R^{2e} is a 6-membered aromatic or heteroaromatic ring that contains 1 or 2 ring N atoms. R^{2e} may be phenyl or pyridyl. R^{2e} may be optionally substituted by 1, 2 or 3 (preferably 1 or 2, preferably 1) halogen, CN, (C1-4)alkoxy or (C1-6)alkyl, the (C1-6)alkyl or (C1-4)alkoxy group being optionally substituted with 1 or more halogen (preferably optionally substituted with 1 or more fluoro) and the (C1-6)alkyl group being optionally substituted with (C1-4)alkoxy. It will be appreciated that alkyl group may be straight chain, branched or cyclic or a combination thereof.

In embodiment (12) of Formula IV or IVa, R^{2e} is a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms. It will be appreciated that R^{2e} may be attached to the rest of the compound through either the 5- to 6-membered aromatic or non-aromatic heterocycle or the 6-membered aromatic or heteroaromatic ring. R^{2e} may be a 9-membered bicyclic ring system comprising a 5-membered aromatic heterocycle containing 1, 2 or 3 ring N heteroatoms and/or 1 O heteroatom fused to a 6-membered aromatic or heteroaromatic ring that contains 0 or 1 ring N atoms; a 9- or 10-membered bicyclic ring system comprising a 5- to 6-membered non-aromatic heterocycle containing 1 or 2 ring O heteroatoms fused to a 6-membered aromatic or heteroaromatic ring that contains 0 or 1 ring N atoms; a 10-membered bicyclic ring system comprising a 6-membered aromatic or non-aromatic heterocycle containing 1 or 2 ring N heteroatoms fused to a 6-membered aromatic or heteroaromatic ring that contains 0, 1 or 2 ring N atoms.

In Formula IV or IVa or any embodiment of Formula IV or IVa, optionally the compound is not selected from a group consisting of

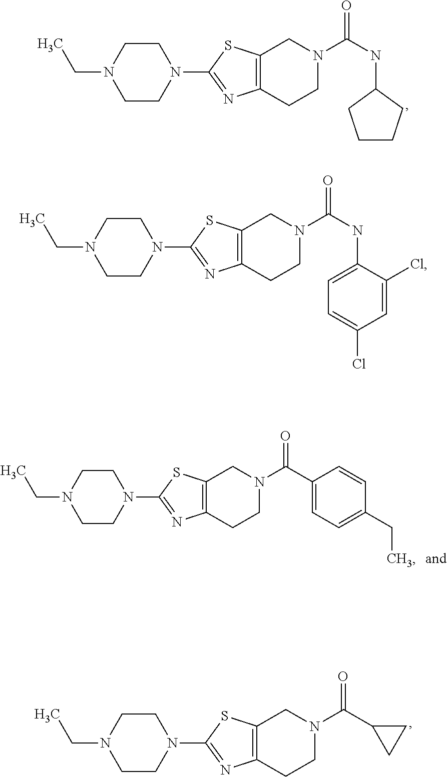 [SMILES: CCN1CCN(c2nc3c(s2)CN(C(=O)NC2CCCC2)CC3)CC1 ; CCN1CCN(c2nc3c(s2)CN(C(=O)Nc2ccc(Cl)cc2Cl)CC3)CC1 ; CCc1ccc(C(=O)N2CCc3nc(N4CCN(CC)CC4)sc3C2)cc1 ; "and" ; CCN1CCN(c2nc3c(s2)CN(C(=O)C2CC2)CC3)CC1]

or a pharmaceutically acceptable salt thereof.

Described herein is a compound of Formula V:

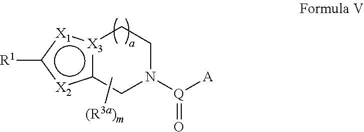

or a pharmaceutically acceptable salt or derivative thereof, wherein:
m is 1, 2, 3 or 4;
and X₁, X₂, X₃, R¹, R^{2a}, R^{2b}, R^{2c}, R^{2f}, R^{3a}, R^{3b}, A, Q, R⁴, R⁵ and a are as defined for Formula I or any of embodiments (1)-(26) and (29)-(34) of Formula I above.

Also described herein is a compound of Formula Va:

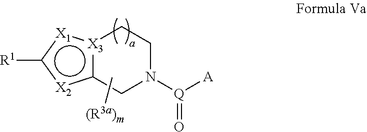

or a pharmaceutically acceptable salt or derivative thereof, wherein:
m is 1, 2, 3 or 4;
and X₁, X₂, X₃, R¹, R^{2a}, R^{2b}, R^{2c}, R^{3a}, R^{3b}, A, Q, R⁴, R⁵ and a are as defined for Formula Ia or any of embodiments (1)-(26) and (29)-(34) of Formula Ia above.

In embodiment (1) of Formula V or Va, m is 1 or 2. Where m is 2, both R^{3a} are preferably on the same ring carbon atom.

In Formula V or Va or any of the embodiments of Formula V or Va, A may be R^{2c} or NR^{2a}R^{2b}.

In a further embodiment of a compound of Formula I to V or Ia to Va or a pharmaceutically acceptable salt thereof, including any of the embodiments thereof described above, one or more hydrogen atoms are replaced by 2H. The remaining moieties may be as defined for any aspect or embodiment of Formula Ia-Va described herein, mutatis mutandis.

Compounds of Formula I to V, including any embodiments thereof, may be preferred.

In an embodiment, the compound of Formula I or Ia is selected from:
1-(2-(3,8-diazabicyclo[3.2.1]octan-8-yl)-6,7-dihydrothiazolo[5,4-c]pyridin-5(4H)-yl)-2-cyclopentylethan-1-one;
1-(2-(3,8-diazabicyclo[3.2.1]octan-8-yl)-6,7-dihydrothiazolo[5,4-c]pyridin-5(4H)-yl)-2-(4,4-difluorocyclohexyl)ethan-1-one;
1-(2-(3,8-diazabicyclo[3.2.1]octan-8-yl)-6,7-dihydrothiazolo[5,4-c]pyridin-5(4H)-yl)-2-(tetrahydro-2H-pyran-4-yl)ethan-1-one;
1-(2-(3,8-diazabicyclo[3.2.1]octan-8-yl)-6,7-dihydrothiazolo[5,4-c]pyridin-5(4H)-yl)-2-isopropoxyethan-1-one;
1-(2-(3,8-diazabicyclo[3.2.1]octan-8-yl)-6,7-dihydrothiazolo[5,4-c]pyridin-5(4H)-yl)-3-phenylpropan-1-one;
1-(2-(3,8-diazabicyclo[3.2.1]octan-8-yl)-6,7-dihydrothiazolo[5,4-c]pyridin-5(4H)-yl)-2-phenoxyethan-1-one;
4-(3-(2-(3,8-diazabicyclo[3.2.1]octan-8-yl)-6,7-dihydrothiazolo[5,4-c]pyridin-5(4H)-yl)-3-oxopropyl)benzonitrile;
1-(2-(3,8-diazabicyclo[3.2.1]octan-8-yl)-6,7-dihydrothiazolo[5,4-c]pyridin-5(4H)-yl)-2-phenylethan-1-one;
1-(2-(3,8-diazabicyclo[3.2.1]octan-8-yl)-6,7-dihydrothiazolo[5,4-c]pyridin-5(4H)-yl)-3-methylbutan-1-one;
1-(2-(3,8-diazabicyclo[3.2.1]octan-8-yl)-6,7-dihydrothiazolo[5,4-c]pyridin-5(4H)-yl)-3,3-dimethylbutan-1-one;
1-(2-(3,8-diazabicyclo[3.2.1]octan-8-yl)-6,7-dihydrothiazolo[5,4-c]pyridin-5(4H)-yl)-2-cyclobutylethan-1-one;
1-(2-(3,8-diazabicyclo[3.2.1]octan-8-yl)-6,7-dihydrothiazolo[5,4-c]pyridin-5(4H)-yl)-2-(bicyclo[1.1.1]pentan-1-yl)ethan-1-one;
1-(2-(3,8-diazabicyclo[3.2.1]octan-8-yl)-6,7-dihydrothiazolo[5,4-c]pyridin-5(4H)-yl)-2-cyclohexylethan-1-one;
(2-(3,8-diazabicyclo[3.2.1]octan-8-yl)-6,7-dihydrothiazolo[5,4-c]pyridin-5(4H)-yl)(4,4-difluorocyclohexyl)methanone;
(2S)-1-(2-(3,8-diazabicyclo[3.2.1]octan-8-yl)-6,7-dihydrothiazolo[5,4-c]pyridin-5(4H)-yl)-2-methylbutan-1-one;
(2R)-1-(2-(3,8-diazabicyclo[3.2.1]octan-8-yl)-6,7-dihydrothiazolo[5,4-c]pyridin-5(4H)-yl)-2-methylbutan-1-one;
1-(2-(3,8-diazabicyclo[3.2.1]octan-8-yl)-6,7-dihydrothiazolo[5,4-c]pyridin-5(4H)-yl)-2,3-dimethylbutan-1-one;
(2S)-1-(2-(3,8-diazabicyclo[3.2.1]octan-8-yl)-6,7-dihydrothiazolo[5,4-c]pyridin-5(4H)-yl)-2,3-dimethylbutan-1-one;
(2R)-1-(2-(3,8-diazabicyclo[3.2.1]octan-8-yl)-6,7-dihydrothiazolo[5,4-c]pyridin-5(4H)-yl)-2,3-dimethylbutan-1-one;
1-(2-(3,8-diazabicyclo[3.2.1]octan-8-yl)-6,7-dihydrothiazolo[5,4-c]pyridin-5(4H)-yl)-2-methylpropan-1-one;
1-(2-(3,8-diazabicyclo[3.2.1]octan-8-yl)-6,7-dihydrothiazolo[5,4-c]pyridin-5(4H)-yl)-2-(4-fluorophenoxy) ethan-1-one;
(2-(3,8-diazabicyclo[3.2.1]octan-8-yl)-6,7-dihydrothiazolo[5,4-c]pyridin-5(4H)-yl)(cyclopentyl)methanone;
1-(2-(3,8-diazabicyclo[3.2.1]octan-8-yl)-6,7-dihydrothiazolo[5,4-c]pyridin-5(4H)-yl)-2-phenoxypropan-1-one;
1-(2-(3,8-diazabicyclo[3.2.1]octan-8-yl)-6,7-dihydrothiazolo[5,4-c]pyridin-5(4H)-yl)-2-cyclopentyl-2,2-difluoroethan-1-one;
1-(2-(3,8-diazabicyclo[3.2.1]octan-8-yl)-6,7-dihydrothiazolo[5,4-c]pyridin-5(4H)-yl)-2-(4-fluorophenyl)ethan-1-one;
1-(2-(3,8-diazabicyclo[3.2.1]octan-8-yl)-6,7-dihydrothiazolo[5,4-c]pyridin-5(4H)-yl)-3,3,3-trifluoropropan-1-one;
1-(2-(3,8-diazabicyclo[3.2.1]octan-8-yl)-6,7-dihydrothiazolo[5,4-c]pyridin-5(4H)-yl)-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-hydroxy-3-methylbutan-1-one;
(2-(3,8-diazabicyclo[3.2.1]octan-8-yl)-6,7-dihydrothiazolo[5,4-c]pyridin-5(4H)-yl)(3,3-difluorocyclopentyl)methanone;
1-(2-(3,8-diazabicyclo[3.2.1]octan-8-yl)-6,7-dihydrothiazolo[5,4-c]pyridin-5(4H)-yl)-2-methyl-2-phenoxypropan-1-one;
(2-(3,8-diazabicyclo[3.2.1]octan-8-yl)-6,7-dihydrothiazolo[5,4-c]pyridin-5(4H)-yl)(1-phenoxycyclopropyl)methanone;
1-(2-(3,8-diazabicyclo[3.2.1]octan-8-yl)-6,7-dihydrothiazolo[5,4-c]pyridin-5(4H)-yl)-3,3,3-trifluoro-2,2-dimethylpropan-1-one;
(2-(3,8-diazabicyclo[3.2.1]octan-8-yl)-6,7-dihydrothiazolo[5,4-c]pyridin-5(4H)-yl)(1-phenylcyclopropyl)methanone;
1-(2-(3,8-diazabicyclo[3.2.1]octan-8-yl)-6,7-dihydrothiazolo[5,4-c]pyridin-5(4H)-yl)-2-cyclopentyl-2-fluoroethan-1-one;
1-(2-(3,8-diazabicyclo[3.2.1]octan-8-yl)-6,7-dihydrothiazolo[5,4-c]pyridin-5(4H)-yl)-2-(4,4-difluorocyclohexyl)-2-fluoroethan-1-one;
1-(2-(3,8-diazabicyclo[3.2.1]octan-8-yl)-6,7-dihydrothiazolo[5,4-c]pyridin-5(4H)-yl)-2,2-difluoro-2-phenylethan-1-one;
(2-(3,8-diazabicyclo[3.2.1]octan-8-yl)-6,7-dihydrothiazolo[5,4-c]pyridin-5(4H)-yl)(2,2-dimethylcyclopentyl)methanone;
(2-(3,8-diazabicyclo[3.2.1]octan-8-yl)-6,7-dihydrothiazolo[5,4-c]pyridin-5(4H)-yl)(3,3-dimethylcyclopentyl)methanone;
2-(3,8-diazabicyclo[3.2.1]octan-8-yl)-N-cyclopentyl-6,7-dihydrothiazolo[5,4-c]pyridine-5(4H)-carboxamide;
2-(3,8-diazabicyclo[3.2.1]octan-8-yl)-N-isopropyl-6,7-dihydrothiazolo[5,4-c]pyridine-5(4H)-carboxamide;
2-(3,8-diazabicyclo[3.2.1]octan-8-yl)-N-cyclohexyl-6,7-dihydrothiazolo[5,4-c]pyridine-5(4H)-carboxamide;
2-(3,8-diazabicyclo[3.2.1]octan-8-yl)-N-cycloheptyl-6,7-dihydrothiazolo[5,4-c]pyridine-5(4H)-carboxamide;
2-(3,8-diazabicyclo[3.2.1]octan-8-yl)-N-(cyclohexylmethyl)-6,7-dihydrothiazolo[5,4-c]pyridine-5(4H)-carboxamide;
2-(3,8-diazabicyclo[3.2.1]octan-8-yl)-N-(4-fluorobenzyl)-6,7-dihydrothiazolo[5,4-c]pyridine-5(4H)-carboxamide;
2-(3,8-diazabicyclo[3.2.1]octan-8-yl)-N-(4-(trifluoromethyl)benzyl)-6,7-dihydrothiazolo[5,4-c]pyridine-5(4H)-carboxamide;
2-(3,8-diazabicyclo[3.2.1]octan-8-yl)-N-phenyl-6,7-dihydrothiazolo[5,4-c]pyridine-5(4H)-carboxamide;
2-(3,8-diazabicyclo[3.2.1]octan-8-yl)-N-(4,4-difluorocyclohexyl)-6,7-dihydrothiazolo[5,4-c]pyridine-5(4H)-carboxamide;
(2-(3,8-diazabicyclo[3.2.1]octan-8-yl)-6,7-dihydrothiazolo[5,4-c]pyridin-5(4H)-yl)(pyrrolidin-1-yl)methanone;
2-(3,8-diazabicyclo[3.2.1]octan-8-yl)-N-cyclopentyl-N-methyl-6,7-dihydrothiazolo[5,4-c]pyridine-5(4H)-carboxamide;
1-(2-(3,8-diazabicyclo[3.2.1]octan-8-yl)-6,7-dihydrothiazolo[5,4-c]pyridin-5(4H)-yl)-2,2-dimethylpropan-1-one;
1-(2-(3,8-diazabicyclo[3.2.1]octan-8-yl)-4,6-dihydro-5H-pyrrolo[3,4-d]thiazol-5-yl)-2-cyclopentylethan-1-one;
1-(2-(3,8-diazabicyclo[3.2.1]octan-8-yl)-4,6-dihydro-5H-pyrrolo[3,4-d]thiazol-5-yl)-2-(4,4-difluorocyclohexyl)ethan-1-one;
2-(3,8-diazabicyclo[3.2.1]octan-8-yl)-N-cyclopentyl-4,6-dihydro-5H-pyrrolo[3,4-d]thiazole-5-carboxamide;
2-(3,8-diazabicyclo[3.2.1]octan-8-yl)-N-(4,4-difluorocyclohexyl)-4,6-dihydro-5H-pyrrolo[3,4-d]thiazole-5-carboxamide;
2-(3,8-diazabicyclo[3.2.1]octan-8-yl)-N-(4,4-difluorocyclohexyl)-6,7-dihydropyrazolo[1,5-a]pyrazine-5(4H)-carboxamide;
1-(2-(3,8-diazabicyclo[3.2.1]octan-8-yl)-6,7-dihydropyrazolo[1,5-a]pyrazin-5(4H)-yl)-2-(4,4-difluorocyclohexyl)ethan-1-one;
1-(2-(3,8-diazabicyclo[3.2.1]octan-8-yl)-6,7-dihydropyrazolo[1,5-a]pyrazin-5(4H)-yl)-2-(4-fluorophenoxy) ethan-1-one;
1-(2-(3,8-diazabicyclo[3.2.1]octan-8-yl)-6,7-dihydrothiazolo[5,4-c]pyridin-5(4H)-yl)-2-((2-fluoropyridin-4-yl)oxy) ethan-1-one;
1-(2-(3,8-diazabicyclo[3.2.1]octan-8-yl)-6,7-dihydrothiazolo[5,4-c]pyridin-5(4H)-yl)-2-((6-fluoropyridin-3-yl)oxy) ethan-1-one;
2-cyclopentyl-1-(2-(3-methyl-3,8-diazabicyclo[3.2.1]octan-8-yl)-6,7-dihydrothiazolo[5,4-c]pyridin-5(4H)-yl)ethan-1-one;
2-(4,4-difluorocyclohexyl)-1-(2-(3-methyl-3,8-diazabicyclo[3.2.1]octan-8-yl)-6,7-dihydrothiazolo[5,4-c]pyridin-5(4H)-yl)ethan-1-one;
N-(4,4-difluorocyclohexyl)-2-(3-methyl-3,8-diazabicyclo[3.2.1]octan-8-yl)-6,7-dihydrothiazolo[5,4-c]pyridine-5(4H)-carboxamide;
N-cyclopentyl-2-(3-methyl-3,8-diazabicyclo[3.2.1]octan-8-yl)-6,7-dihydrothiazolo[5,4-c]pyridine-5(4H)-carboxamide;
2-((2-fluoropyridin-4-yl)oxy)-1-(2-(3-methyl-3,8-diazabicyclo[3.2.1]octan-8-yl)-6,7-dihydrothiazolo[5,4-c]pyridin-5(4H)-yl)ethan-1-one;
2-((6-fluoropyridin-3-yl)oxy)-1-(2-(3-methyl-3,8-diazabicyclo[3.2.1]octan-8-yl)-6,7-dihydrothiazolo[5,4-c]pyridin-5(4H)-yl)ethan-1-one;
N-(4,4-difluorocyclohexyl)-2-(3-methyl-3,8-diazabicyclo[3.2.1]octan-8-yl)-4,6-dihydro-5H-pyrrolo[3,4-d]thiazole-5-carboxamide;
N-cyclopentyl-2-(3-methyl-3,8-diazabicyclo[3.2.1]octan-8-yl)-4,6-dihydro-5H-pyrrolo[3,4-d]thiazole-5-carboxamide;
2-cyclopentyl-1-(2-(3-methyl-3,8-diazabicyclo[3.2.1]octan-8-yl)-4,6-dihydro-5H-pyrrolo[3,4-d]thiazol-5-yl)ethan-1-one;
2-(4,4-difluorocyclohexyl)-1-(2-(3-methyl-3,8-diazabicyclo[3.2.1]octan-8-yl)-4,6-dihydro-5H-pyrrolo[3,4-d]thiazol-5-yl)ethan-1-one;
N-(4,4-difluorocyclohexyl)-2-(3-methyl-3,8-diazabicyclo[3.2.1]octan-8-yl)-6,7-dihydropyrazolo[1,5-a]pyrazine-5(4H)-carboxamide;
2-(4,4-difluorocyclohexyl)-1-(2-(3-methyl-3,8-diazabicyclo[3.2.1]octan-8-yl)-6,7-dihydropyrazolo[1,5-a]pyrazin-5(4H)-yl)ethan-1-one;
1-(2-(3,8-diazabicyclo[3.2.1]octan-8-yl)-5,6-dihydro-[1,2,4]triazolo[1,5-a]pyrazin-7(8H)-yl)-2-(4,4-difluorocyclohexyl)ethan-1-one;
1-(2-(3,8-diazabicyclo[3.2.1]octan-8-yl)-7,7-dimethyl-6,7-dihydrothiazolo[5,4-c]pyridin-5(4H)-yl)-2-(4,4-difluorocyclohexyl)ethan-1-one;
1-(2-(3,8-diazabicyclo[3.2.1]octan-8-yl)-7,7-dimethyl-6,7-dihydrothiazolo[5,4-c]pyridin-5(4H)-yl)-2-cyclopentylethan-1-one;
2-(4,4-difluorocyclohexyl)-1-(7,7-dimethyl-2-(piperazin-1-yl)-6,7-dihydrothiazolo[5,4-c]pyridin-5(4H)-yl)ethan-1-one;
2-cyclopentyl-1-(7,7-dimethyl-2-(piperazin-1-yl)-6,7-dihydrothiazolo[5,4-c]pyridin-5(4H)-yl)ethan-1-one;
2-cyclopentyl-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one;
2-(4,4-difluorocyclohexyl)-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one;
2-(4,4-difluorocyclohexyl)-1-(7,7-dimethyl-2-(4-methylpiperazin-1-yl)-6,7-dihydrothiazolo[5,4-c]pyridin-5(4H)-yl)ethan-1-one;
2-(3,8-diazabicyclo[3.2.1]octan-8-yl)-5-((cyclopentylmethyl) sulfonyl)-4,5,6,7-tetrahydrothiazolo[5,4-c]pyridine;
2-(3,8-diazabicyclo[3.2.1]octan-8-yl)-N-cyclopentyl-6,7-dihydrothiazolo[5,4-c]pyridine-5(4H)-sulfonamide;
2-cyclopentyl-1-(2-(piperazin-1-yl)-6,7-dihydrothiazolo[5,4-c]pyridin-5(4H)-yl)ethan-1-one; 2-(4-fluorophenoxy)-1-(2-(piperazin-1-yl)-6,7-dihydrothiazolo[5,4-c]pyridin-5(4H)-yl)ethan-1-one;
(S)-2-cyclopentyl-1-(2-(3-(hydroxymethyl)piperazin-1-yl)-6,7-dihydrothiazolo[5,4-c]pyridin-5(4H)-yl)ethan-1-one;
(R)-2-cyclopentyl-1-(2-(3-(hydroxymethyl)piperazin-1-yl)-6,7-dihydrothiazolo[5,4-c]pyridin-5(4H)-yl)ethan-1-one;
2-cyclopentyl-1-(2-(piperidin-4-yl)-6,7-dihydrothiazolo[5,4-c]pyridin-5(4H)-yl)ethan-1-one; 1-(2-(3,8-diazabicyclo[3.2.1]octan-8-yl)-6,7-dihydrothiazolo[5,4-c]pyridin-5(4H)-yl)-2-(3,3-dimethylcyclobutyl)ethan-1-one;
(2-(3,8-diazabicyclo[3.2.1]octan-8-yl)-6,7-dihydrothiazolo[5,4-c]pyridin-5(4H)-yl)(2,2-dimethylpyrrolidin-1-yl)methanone;
(2-(3,8-diazabicyclo[3.2.1]octan-8-yl)-6,7-dihydrothiazolo[5,4-c]pyridin-5(4H)-yl)(3,3-dimethylpyrrolidin-1-yl)methanone;
(2-(3,8-diazabicyclo[3.2.1]octan-8-yl)-6,7-dihydrothiazolo[5,4-c]pyridin-5(4H)-yl)(2-azaspiro[4.4]nonan-2-yl)methanone;
(2-(3,8-diazabicyclo[3.2.1]octan-8-yl)-6,7-dihydrothiazolo[5,4-c]pyridin-5(4H)-yl)(6-azaspiro[3.4]octan-6-yl)methanone;
2-(3,8-diazabicyclo[3.2.1]octan-8-yl)-N-(1-methylcyclobutyl)-6,7-dihydrothiazolo[5,4-c]pyridine-5(4H)-carboxamide;
2-(3,8-diazabicyclo[3.2.1]octan-8-yl)-N-(1-methylcyclopentyl)-6,7-dihydrothiazolo[5,4-c]pyridine-5(4H)-carboxamide;
1-(2-(3,8-diazabicyclo[3.2.1]octan-8-yl)-6,7-dihydropyrazolo[1,5-a]pyrazin-5(4H)-yl)-2,2-difluoro-2-phenylethan-1-one;
1-(2-(3,8-diazabicyclo[3.2.1]octan-8-yl)-6,7-dihydropyrazolo[1,5-a]pyrazin-5(4H)-yl)-2-cyclopentylethan-1-one;
1-(2-(3,8-diazabicyclo[3.2.1]octan-8-yl)-6,7-dihydropyrazolo[1,5-a]pyrazin-5(4H)-yl)-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-(2-(2-methoxyethoxy) phenoxy) ethan-1-one;
1-(2-(3,8-diazabicyclo[3.2.1]octan-8-yl)-6,7-dihydrothiazolo[5,4-c]pyridin-5(4H)-yl)-2-(1-methylcyclobutyl)ethan-1-one;
1-(2-(3,8-diazabicyclo[3.2.1]octan-8-yl)-6,7-dihydrothiazolo[5,4-c]pyridin-5(4H)-yl)-2-(4-fluorophenyl)-2-methoxyethan-1-one;
1-(2-(3,8-diazabicyclo[3.2.1]octan-8-yl)-6,7-dihydrothiazolo[5,4-c]pyridin-5(4H)-yl)-2-(4-fluorophenyl)-2-hydroxyethan-1-one;
(2-(3,8-diazabicyclo[3.2.1]octan-8-yl)-6,7-dihydrothiazolo[5,4-c]pyridin-5(4H)-yl)(tetrahydrofuran-2-yl)methanone;
1-(2-(3,8-diazabicyclo[3.2.1]octan-8-yl)-6,7-dihydrothiazolo[5,4-c]pyridin-5(4H)-yl)-2-(1-methoxycyclopentyl)ethan-1-one;
1-(2-(3,8-diazabicyclo[3.2.1]octan-8-yl)-6,7-dihydrothiazolo[5,4-c]pyridin-5(4H)-yl)-2-(1-(methoxymethyl)cyclopentyl)ethan-1-one;
1-(2-(3,8-diazabicyclo[3.2.1]octan-8-yl)-6,7-dihydrothiazolo[5,4-c]pyridin-5(4H)-yl)-2-(pyrazin-2-yl)ethan-1-one;
1-(2-(3,8-diazabicyclo[3.2.1]octan-8-yl)-6,7-dihydrothiazolo[5,4-c]pyridin-5(4H)-yl)-2-(3,3-difluorocyclopentyl)ethan-1-one;
1-(2-(3,8-diazabicyclo[3.2.1]octan-8-yl)-6,7-dihydrothiazolo[5,4-c]pyridin-5(4H)-yl)-2-(2-methoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2-methoxyphenyl)ethan-1-one;
1-(2-(3,8-diazabicyclo[3.2.1]octan-8-yl)-6,7-dihydrothiazolo[5,4-c]pyridin-5(4H)-yl)-2-(4-fluoro-2-methoxyphenyl)ethan-1-one;
1-(2-(3,8-diazabicyclo[3.2.1]octan-8-yl)-6,7-dihydrothiazolo[5,4-c]pyridin-5(4H)-yl)-2-(2-fluoropyridin-4-yl)ethan-1-one;
1-(2-(3,8-diazabicyclo[3.2.1]octan-8-yl)-6,7-dihydrothiazolo[5,4-c]pyridin-5(4H)-yl)-3-(6-fluoropyridin-3-yl) propan-1-one;
1-(2-(3,8-diazabicyclo[3.2.1]octan-8-yl)-6,7-dihydrothiazolo[5,4-c]pyridin-5(4H)-yl)-2-(4-fluoro-2-methoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2,4-difluorophenyl)ethan-1-one;
1-(2-(3,8-diazabicyclo[3.2.1]octan-8-yl)-6,7-dihydrothiazolo[5,4-c]pyridin-5(4H)-yl)-2-(4-fluoro-2-methylphenoxy) ethan-1-one;
1-(2-(3,8-diazabicyclo[3.2.1]octan-8-yl)-6,7-dihydrothiazolo[5,4-c]pyridin-5(4H)-yl)-2-cyclopentyl-2-methylpropan-1-one;
1-(2-(3,8-diazabicyclo[3.2.1]octan-8-yl)-6,7-dihydrothiazolo[5,4-c]pyridin-5(4H)-yl)-3-(4-fluoro-2-methoxyphenyl)propan-1-one;
1-(2-(3,8-diazabicyclo[3.2.1]octan-8-yl)-6,7-dihydrothiazolo[5,4-c]pyridin-5(4H)-yl)-2-(2,4-difluorophenoxy)ethan-1-one;
1-(2-(3,8-diazabicyclo[3.2.1]octan-8-yl)-6,7-dihydrothiazolo[5,4-c]pyridin-5(4H)-yl)-2-(cyclopentyloxy)ethan-1-one;
(2-(3,8-diazabicyclo[3.2.1]octan-8-yl)-6,7-dihydrothiazolo[5,4-c]pyridin-5(4H)-yl)(1-methylcyclopentyl)methanone;
(2-(3,8-diazabicyclo[3.2.1]octan-8-yl)-6,7-dihydrothiazolo[5,4-c]pyridin-5(4H)-yl)(1-fluorocyclopentyl)methanone;
3-(2-(3,8-diazabicyclo[3.2.1]octan-8-yl)-6,7-dihydrothiazolo[5,4-c]pyridin-5(4H)-yl)-2-cyclopentyl-3-oxopropanenitrile;

1-(2-(3,8-diazabicyclo[3.2.1]octan-8-yl)-6,7-dihydrothiazolo[5,4-c]pyridin-5(4H)-yl)-2-(2,6-dimethylpyridin-4-yl)ethan-1-one;
1-(2-(3,8-diazabicyclo[3.2.1]octan-8-yl)-6,7-dihydrothiazolo[5,4-c]pyridin-5(4H)-yl)-2,2-dimethylbutan-1-one;
1-(2-(3,8-diazabicyclo[3.2.1]octan-8-yl)-6,7-dihydrothiazolo[5,4-c]pyridin-5(4H)-yl)-2-(2-oxaspiro[3.5]nonan-7-yl)ethan-1-one;
1-(2-(3,8-diazabicyclo[3.2.1]octan-8-yl)-6,7-dihydrothiazolo[5,4-c]pyridin-5(4H)-yl)-2-(4-fluoro-2-methylphenyl)ethan-1-one;
1-(2-(3,8-diazabicyclo[3.2.1]octan-8-yl)-6,7-dihydrothiazolo[5,4-c]pyridin-5(4H)-yl)-2-cyclopentyl-2-methoxyethan-1-one;
1-(2-(3,8-diazabicyclo[3.2.1]octan-8-yl)-6,7-dihydrothiazolo[5,4-c]pyridin-5(4H)-yl)-2,3,3-trimethylbutan-1-one (enantiomer-1);

1-(2-(3,8-diazabicyclo[3.2.1]octan-8-yl)-6,7-dihydrothiazolo[5,4-c]pyridin-5(4H)-yl)-2,3,3-trimethylbutan-1-one (enantiomer-2);

1-(2-(3,8-diazabicyclo[3.2.1]octan-8-yl)-6,7-dihydrothiazolo[5,4-c]pyridin-5(4H)-yl)-2-(bicyclo[2.2.1]heptan-1-yl)ethan-1-one;
1-(2-(3,8-diazabicyclo[3.2.1]octan-8-yl)-6,7-dihydrothiazolo[5,4-c]pyridin-5(4H)-yl)-2,2,3-trimethylbutan-1-one;
(2-(3,8-diazabicyclo[3.2.1]octan-8-yl)-6,7-dihydrothiazolo[5,4-c]pyridin-5(4H)-yl)(spiro[2.4]heptan-1-yl)methanone;
1-(2-(3,8-diazabicyclo[3.2.1]octan-8-yl)-6,7-dihydrothiazolo[5,4-c]pyridin-5(4H)-yl)-2-(2-isopropoxyphenoxy) ethan-1-one;
1-(2-(3,8-diazabicyclo[3.2.1]octan-8-yl)-6,7-dihydrothiazolo[5,4-c]pyridin-5(4H)-yl)-2-(2-(trifluoromethoxy)phenoxy)ethan-1-one;
1-(2-(3,8-diazabicyclo[3.2.1]octan-8-yl)-6,7-dihydrothiazolo[5,4-c]pyridin-5(4H)-yl)-2-(6-fluoropyridin-3-yl)ethan-1-one;
1-(2-(3,8-diazabicyclo[3.2.1]octan-8-yl)-6,7-dihydrothiazolo[5,4-c]pyridin-5(4H)-yl)-2,2-difluoro-2-(6-fluoropyridin-3-yl)ethan-1-one;
2-(2-(2-(3,8-diazabicyclo[3.2.1]octan-8-yl)-6,7-dihydrothiazolo[5,4-c]pyridin-5(4H)-yl)-2-oxoethoxy)benzonitrile;
2-(2-(2-(3,8-diazabicyclo[3.2.1]octan-8-yl)-6,7-dihydrothiazolo[5,4-c]pyridin-5(4H)-yl)-2-oxoethoxy)-3-methoxybenzonitrile;
1-(2-(3,8-diazabicyclo[3.2.1]octan-8-yl)-6,7-dihydrothiazolo[5,4-c]pyridin-5(4H)-yl)-2-(cyclopentyloxy)propan-1-one;
(2S)-1-(2-(3,8-diazabicyclo[3.2.1]octan-8-yl)-6,7-dihydrothiazolo[5,4-c]pyridin-5(4H)-yl)-2-cyclopentyl-2-hydroxyethan-1-one;
(2R)-1-(2-(3,8-diazabicyclo[3.2.1]octan-8-yl)-6,7-dihydrothiazolo[5,4-c]pyridin-5(4H)-yl)-2-cyclopentyl-2-hydroxyethan-1-one;
1-(2-(3,8-diazabicyclo[3.2.1]octan-8-yl)-6,7-dihydrothiazolo[5,4-c]pyridin-5(4H)-yl)-2-(2,6-dimethoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2-(difluoromethyl)phenoxy)ethan-1-one;
1-(2-(3,8-diazabicyclo[3.2.1]octan-8-yl)-6,7-dihydrothiazolo[5,4-c]pyridin-5(4H)-yl)-3,3-dimethylpentan-1-one;
1-(2-(3,8-diazabicyclo[3.2.1]octan-8-yl)-6,7-dihydrothiazolo[5,4-c]pyridin-5(4H)-yl)-4,4-difluoro-3,3-dimethylbutan-1-one;
(2R)-1-(2-(3,8-diazabicyclo[3.2.1]octan-8-yl)-6,7-dihydrothiazolo[5,4-c]pyridin-5(4H)-yl)-2-hydroxy-3,3-dimethylbutan-1-one;
(2R)-1-(2-(3,8-diazabicyclo[3.2.1]octan-8-yl)-6,7-dihydrothiazolo[5,4-c]pyridin-5(4H)-yl)-2-cyclohexyl-2-hydroxyethan-1-one;
4-(2-(3,8-diazabicyclo[3.2.1]octan-8-yl)-6,7-dihydrothiazolo[5,4-c]pyridin-5(4H)-yl)-2,2-dimethyl-4-oxobutanenitrile;

(2-(3,8-diazabicyclo[3.2.1]octan-8-yl)-6,7-dihydrothiazolo[5,4-c]pyridin-5(4H)-yl)(2,3-dihydrobenzofuran-2-yl)methanone;
(2-(3,8-diazabicyclo[3.2.1]octan-8-yl)-6,7-dihydrothiazolo[5,4-c]pyridin-5(4H)-yl)(2,3-dihydrobenzo[b][1,4]dioxin-2-yl)methanone;
(2-(3,8-diazabicyclo[3.2.1]octan-8-yl)-6,7-dihydrothiazolo[5,4-c]pyridin-5(4H)-yl)(chroman-2-yl)methanone;
(2-(3,8-diazabicyclo[3.2.1]octan-8-yl)-6,7-dihydrothiazolo[5,4-c]pyridin-5(4H)-yl)(chroman-3-yl)methanone;
(2-(3,8-diazabicyclo[3.2.1]octan-8-yl)-6,7-dihydrothiazolo[5,4-c]pyridin-5(4H)-yl)(isochroman-3-yl)methanone;
(2-(3,8-diazabicyclo[3.2.1]octan-8-yl)-6,7-dihydrothiazolo[5,4-c]pyridin-5(4H)-yl)(2,3-dihydrobenzofuran-3-yl)methanone;
(2-(3,8-diazabicyclo[3.2.1]octan-8-yl)-6,7-dihydrothiazolo[5,4-c]pyridin-5(4H)-yl)(benzofuran-2-yl)methanone;
(2-(3,8-diazabicyclo[3.2.1]octan-8-yl)-6,7-dihydrothiazolo[5,4-c]pyridin-5(4H)-yl)(isoquinolin-1-yl)methanone;
(2-(3,8-diazabicyclo[3.2.1]octan-8-yl)-6,7-dihydrothiazolo[5,4-c]pyridin-5(4H)-yl)(benzofuran-3-yl)methanone;
(2-(3,8-diazabicyclo[3.2.1]octan-8-yl)-6,7-dihydrothiazolo[5,4-c]pyridin-5(4H)-yl)(1H-indol-2-yl)methanone;
(2-(3,8-diazabicyclo[3.2.1]octan-8-yl)-6,7-dihydrothiazolo[5,4-c]pyridin-5(4H)-yl)(1-methyl-1H-indol-2-yl)methanone;
(2-(3,8-diazabicyclo[3.2.1]octan-8-yl)-6,7-dihydrothiazolo[5,4-c]pyridin-5(4H)-yl)(benzo[d]oxazol-2-yl)methanone;
(2-(3,8-diazabicyclo[3.2.1]octan-8-yl)-6,7-dihydrothiazolo[5,4-c]pyridin-5(4H)-yl)(isoquinolin-3-yl)methanone;
(2-(3,8-diazabicyclo[3.2.1]octan-8-yl)-6,7-dihydrothiazolo[5,4-c]pyridin-5(4H)-yl)(phenyl)methanone;
(2-(3,8-diazabicyclo[3.2.1]octan-8-yl)-6,7-dihydrothiazolo[5,4-c]pyridin-5(4H)-yl)(o-tolyl)methanone;
(2-(3,8-diazabicyclo[3.2.1]octan-8-yl)-6,7-dihydrothiazolo[5,4-c]pyridin-5(4H)-yl)(m-tolyl)methanone;
(2-(3,8-diazabicyclo[3.2.1]octan-8-yl)-6,7-dihydrothiazolo[5,4-c]pyridin-5(4H)-yl)(p-tolyl)methanone;
(2-(3,8-diazabicyclo[3.2.1]octan-8-yl)-6,7-dihydrothiazolo[5,4-c]pyridin-5(4H)-yl)(2-methoxyphenyl)methanone;
(2-(3,8-diazabicyclo[3.2.1]octan-8-yl)-6,7-dihydrothiazolo[5,4-c]pyridin-5(4H)-yl)(4-fluorophenyl)methanone;
(2-(3,8-diazabicyclo[3.2.1]octan-8-yl)-6,7-dihydrothiazolo[5,4-c]pyridin-5(4H)-yl)(3-(difluoromethyl)phenyl)methanone;
(2-(3,8-diazabicyclo[3.2.1]octan-8-yl)-6,7-dihydrothiazolo[5,4-c]pyridin-5(4H)-yl)(6-fluoropyridin-3-yl)methanone;
(2-(3,8-diazabicyclo[3.2.1]octan-8-yl)-6,7-dihydrothiazolo[5,4-c]pyridin-5(4H)-yl)(pyridin-2-yl)methanone;
(2-(3,8-diazabicyclo[3.2.1]octan-8-yl)-6,7-dihydrothiazolo[5,4-c]pyridin-5(4H)-yl)(5-fluoropyridin-2-yl)methanone;
(2-(3,8-diazabicyclo[3.2.1]octan-8-yl)-6,7-dihydrothiazolo[5,4-c]pyridin-5(4H)-yl)(2-(difluoromethoxy)phenyl)methanone;
(2-(3,8-diazabicyclo[3.2.1]octan-8-yl)-6,7-dihydrothiazolo[5,4-c]pyridin-5(4H)-yl)(4-(difluoromethoxy)phenyl)methanone;
(2-(3,8-diazabicyclo[3.2.1]octan-8-yl)-6,7-dihydrothiazolo[5,4-c]pyridin-5(4H)-yl)(3-(trifluoromethoxy)phenyl)methanone;
(2-(3,8-diazabicyclo[3.2.1]octan-8-yl)-6,7-dihydrothiazolo[5,4-c]pyridin-5(4H)-yl)(3-(difluoromethoxy)phenyl)methanone;
(2-(3,8-diazabicyclo[3.2.1]octan-8-yl)-6,7-dihydrothiazolo[5,4-c]pyridin-5(4H)-yl)(2-(trifluoromethoxy)phenyl)methanone;
(2-(3,8-diazabicyclo[3.2.1]octan-8-yl)-6,7-dihydrothiazolo[5,4-c]pyridin-5(4H)-yl)(4-(trifluoromethoxy)phenyl)methanone;
(2-(3,8-diazabicyclo[3.2.1]octan-8-yl)-6,7-dihydrothiazolo[5,4-c]pyridin-5(4H)-yl)(2-fluorophenyl)methanone;
(2-(3,8-diazabicyclo[3.2.1]octan-8-yl)-6,7-dihydrothiazolo[5,4-c]pyridin-5(4H)-yl)(2-chlorophenyl)methanone;
(2-(3,8-diazabicyclo[3.2.1]octan-8-yl)-6,7-dihydrothiazolo[5,4-c]pyridin-5(4H)-yl)(2-isopropoxyphenyl)methanone;
(2-(3,8-diazabicyclo[3.2.1]octan-8-yl)-6,7-dihydrothiazolo[5,4-c]pyridin-5(4H)-yl)(2,4-difluorophenyl)methanone;
2-(2-(3,8-diazabicyclo[3.2.1]octan-8-yl)-4,5,6,7-tetrahydrothiazolo[5,4-c]pyridine-5-carbonyl)benzonitrile;

(2-(3,8-diazabicyclo[3.2.1]octan-8-yl)-6,7-dihydrothiazolo[5,4-c]pyridin-5(4H)-yl)(2-(methoxymethyl)phenyl)methanone;
(2-(3,8-diazabicyclo[3.2.1]octan-8-yl)-6,7-dihydrothiazolo[5,4-c]pyridin-5(4H)-yl)(2-isopropylphenyl)methanone;
(2-(3,8-diazabicyclo[3.2.1]octan-8-yl)-6,7-dihydrothiazolo[5,4-c]pyridin-5(4H)-yl)(4-fluoro-2-methylphenyl)methanone;
(2-(3,8-diazabicyclo[3.2.1]octan-8-yl)-6,7-dihydrothiazolo[5,4-c]pyridin-5(4H)-yl)(chroman-8-yl)methanone;
(2-(3,8-diazabicyclo[3.2.1]octan-8-yl)-6,7-dihydrothiazolo[5,4-c]pyridin-5(4H)-yl)(2-(difluoromethyl)phenyl)methanone;
(2-(3,8-diazabicyclo[3.2.1]octan-8-yl)-6,7-dihydrothiazolo[5,4-c]pyridin-5(4H)-yl)(2-methoxy-4-methylphenyl)methanone;
(2-(3,8-diazabicyclo[3.2.1]octan-8-yl)-6,7-dihydrothiazolo[5,4-c]pyridin-5(4H)-yl)(2-cyclopropylphenyl)methanone;
(2-(3,8-diazabicyclo[3.2.1]octan-8-yl)-6,7-dihydrothiazolo[5,4-c]pyridin-5(4H)-yl)(6-chloropyridin-3-yl)methanone;
(2-(3,8-diazabicyclo[3.2.1]octan-8-yl)-6,7-dihydrothiazolo[5,4-c]pyridin-5(4H)-yl)(4-fluoro-2-methoxyphenyl)methanone;
(2-(3,8-diazabicyclo[3.2.1]octan-8-yl)-6,7-dihydrothiazolo[5,4-c]pyridin-5(4H)-yl)(2-(1,1-difluoroethyl)phenyl)methanone;
(2-(3,8-diazabicyclo[3.2.1]octan-8-yl)-6,7-dihydrothiazolo[5,4-c]pyridin-5(4H)-yl)(2-chloropyridin-3-yl)methanone;
(2-(3,8-diazabicyclo[3.2.1]octan-8-yl)-6,7-dihydrothiazolo[5,4-c]pyridin-5(4H)-yl)(2,4-dimethylphenyl)methanone;
(2-(3,8-diazabicyclo[3.2.1]octan-8-yl)-6,7-dihydrothiazolo[5,4-c]pyridin-5(4H)-yl)(4-chlorophenyl)methanone;
(2-(3,8-diazabicyclo[3.2.1]octan-8-yl)-6,7-dihydrothiazolo[5,4-c]pyridin-5(4H)-yl)(4-(trifluoromethyl)phenyl)methanone;
(2-(3,8-diazabicyclo[3.2.1]octan-8-yl)-6,7-dihydrothiazolo[5,4-c]pyridin-5(4H)-yl)(4-cyclopropylphenyl)methanone;
(2-(3,8-diazabicyclo[3.2.1]octan-8-yl)-6,7-dihydrothiazolo[5,4-c]pyridin-5(4H)-yl)(4-(difluoromethyl)phenyl)methanone;
(2-(3,8-diazabicyclo[3.2.1]octan-8-yl)-6,7-dihydrothiazolo[5,4-c]pyridin-5(4H)-yl)(2,3-dihydrobenzofuran-7-yl)methanone;
(2-(3,8-diazabicyclo[3.2.1]octan-8-yl)-6,7-dihydrothiazolo[5,4-c]pyridin-5(4H)-yl)(4-(2,2-difluoroethyl)phenyl)methanone;
(2-(3,8-diazabicyclo[3.2.1]octan-8-yl)-6,7-dihydrothiazolo[5,4-c]pyridin-5(4H)-yl)(4-(2,2,2-trifluoroethyl)phenyl)methanone;
(2-(3,8-diazabicyclo[3.2.1]octan-8-yl)-6,7-dihydrothiazolo[5,4-c]pyridin-5(4H)-yl)(2,2-difluorobenzo[d][1,3]dioxol-4-yl)methanone;
(2-(3,8-diazabicyclo[3.2.1]octan-8-yl)-6,7-dihydrothiazolo[5,4-c]pyridin-5(4H)-yl)(benzo[d][1,3]dioxol-4-yl)methanone;
(2-(3,8-diazabicyclo[3.2.1]octan-8-yl)-6,7-dihydrothiazolo[5,4-c]pyridin-5(4H)-yl)(3-fluorophenyl)methanone;
(2-(3,8-diazabicyclo[3.2.1]octan-8-yl)-6,7-dihydrothiazolo[5,4-c]pyridin-5(4H)-yl)(2,3-dihydrobenzofuran-4-yl)methanone;
(2-(3,8-diazabicyclo[3.2.1]octan-8-yl)-6,7-dihydrothiazolo[5,4-c]pyridin-5(4H)-yl)(quinolin-8-yl)methanone;
2-(2-(3,8-diazabicyclo[3.2.1]octan-8-yl)-4,5,6,7-tetrahydrothiazolo[5,4-c]pyridine-5-carbonyl)-5-methylbenzonitrile;
4-(2-(3,8-diazabicyclo[3.2.1]octan-8-yl)-4,5,6,7-tetrahydrothiazolo[5,4-c]pyridine-5-carbonyl)benzonitrile;

(2-(3,8-diazabicyclo[3.2.1]octan-8-yl)-6,7-dihydrothiazolo[5,4-c]pyridin-5(4H)-yl)(6-fluoro-2-methoxypyridin-3-yl)methanone;
(2-(3,8-diazabicyclo[3.2.1]octan-8-yl)-6,7-dihydrothiazolo[5,4-c]pyridin-5(4H)-yl)(6-(trifluoromethyl)pyridin-3-yl)methanone;
(2-(3,8-diazabicyclo[3.2.1]octan-8-yl)-6,7-dihydrothiazolo[5,4-c]pyridin-5(4H)-yl)(6-fluoro-4-methylpyridin-3-yl)methanone;
(2-(3,8-diazabicyclo[3.2.1]octan-8-yl)-6,7-dihydrothiazolo[5,4-c]pyridin-5(4H)-yl)(6-(difluoromethyl)pyridin-3-yl)methanone;
cyclopentyl 2-(3,8-diazabicyclo[3.2.1]octan-8-yl)-6,7-dihydrothiazolo[5,4-c]pyridine-5(4H)-carboxylate;
isopropyl 2-(3,8-diazabicyclo[3.2.1]octan-8-yl)-6,7-dihydrothiazolo[5,4-c]pyridine-5(4H)-carboxylate;
benzyl 2-(3,8-diazabicyclo[3.2.1]octan-8-yl)-6,7-dihydrothiazolo[5,4-c]pyridine-5(4H)-carboxylate;
phenyl 2-(3,8-diazabicyclo[3.2.1]octan-8-yl)-6,7-dihydrothiazolo[5,4-c]pyridine-5(4H)-carboxylate;
1-(2-(3,8-diazabicyclo[3.2.1]octan-8-yl)-6,7-dihydrothiazolo[5,4-c]pyridin-5(4H)-yl)-2-hydroxy-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-methoxy-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-(2-(2-hydroxyethoxy) phenoxy) ethan-1-one;
1-(2-(3,8-diazabicyclo[3.2.1]octan-8-yl)-6,7-dihydrothiazolo[5,4-c]pyridin-5(4H)-yl)-2,2-difluoro-2-(2-fluoropyridin-4-yl)ethan-1-one;
1-(2-(3,8-diazabicyclo[3.2.1]octan-8-yl)-6,7-dihydrothiazolo[5,4-c]pyridin-5(4H)-yl)-2-(2-(difluoromethoxy) phenoxy) ethan-1-one;
2-(2-(2-(3,8-diazabicyclo[3.2.1]octan-8-yl)-6,7-dihydrothiazolo[5,4-c]pyridin-5(4H)-yl)-2-oxoethoxy)-5-fluoro-3-methoxybenzonitrile;
1-(2-(3,8-diazabicyclo[3.2.1]octan-8-yl)-6,7-dihydrothiazolo[5,4-c]pyridin-5(4H)-yl)-2-cyclopentyl-2-hydroxypropan-1-one (enantiomer-1);
1-(2-(3,8-diazabicyclo[3.2.1]octan-8-yl)-6,7-dihydrothiazolo[5,4-c]pyridin-5(4H)-yl)-2-cyclopentyl-2-hydroxypropan-1-one (enantiomer-2);
(2-(3,8-diazabicyclo[3.2.1]octan-8-yl)-7,7-dimethyl-6,7-dihydrothiazolo[5,4-c]pyridin-5(4H)-yl)(cyclopentyl)methanone;
1-(2-(3,8-diazabicyclo[3.2.1]octan-8-yl)-7,7-dimethyl-6,7-dihydrothiazolo[5,4-c]pyridin-5(4H)-yl)-3-methylbutan-1-one;
1-(2-(3,8-diazabicyclo[3.2.1]octan-8-yl)-7,7-dimethyl-6,7-dihydrothiazolo[5,4-c]pyridin-5(4H)-yl)-2-methylpropan-1-one;
1-(2-(3,8-diazabicyclo[3.2.1]octan-8-yl)-7,7-dimethyl-6,7-dihydrothiazolo[5,4-c]pyridin-5(4H)-yl)-2-hydroxy-3-methylbutan-1-one;
1-(2-(3,8-diazabicyclo[3.2.1]octan-8-yl)-7,7-dimethyl-6,7-dihydrothiazolo[5,4-c]pyridin-5(4H)-yl)-2-(2-oxaspiro[3.5]nonan-7-yl)ethan-1-one;
3-(2-(3,8-diazabicyclo[3.2.1]octan-8-yl)-7,7-dimethyl-6,7-dihydrothiazolo[5,4-c]pyridin-5(4H)-yl)-2-cyclopentyl-3-oxopropanenitrile;
(2R)-1-(2-(3,8-diazabicyclo[3.2.1]octan-8-yl)-7,7-dimethyl-6,7-dihydrothiazolo[5,4-c]pyridin-5(4H)-yl)-2-cyclopentyl-2-hydroxyethan-1-one;
(2-(3,8-diazabicyclo[3.2.1]octan-8-yl)-7,7-dimethyl-6,7-dihydrothiazolo[5,4-c]pyridin-5(4H)-yl)(phenyl)methanone;
(2-(3,8-diazabicyclo[3.2.1]octan-8-yl)-7,7-dimethyl-6,7-dihydrothiazolo[5,4-c]pyridin-5(4H)-yl)(6-fluoropyridin-3-yl)methanone;
1-(2′-(3,8-diazabicyclo[3.2.1]octan-8-yl)-4′H-spiro[cyclopropane-1,7′-thiazolo[5,4-c]pyridin]-5′ (6′H)-yl)-2-cyclopentylethan-1-one;
1-(2′-(3,8-diazabicyclo[3.2.1]octan-8-yl)-2,3,5,6-tetrahydro-4′H-spiro[pyran-4,7′-thiazolo[5,4-c]pyridin]-5′ (6′H)-yl)-2-cyclopentylethan-1-one;
1-(2-(3,8-diazabicyclo[3.2.1]octan-8-yl)-6,6-dimethyl-6,7-dihydrothiazolo[5,4-c]pyridin-5(4H)-yl)-2-cyclopentylethan-1-one;
1-(2-(3,8-diazabicyclo[3.2.1]octan-8-yl)-6,6-dimethyl-6,7-dihydrothiazolo[5,4-c]pyridin-5(4H)-yl)-2-methylpropan-1-one;
(2-(3,8-diazabicyclo[3.2.1]octan-8-yl)-6,6-dimethyl-6,7-dihydrothiazolo[5,4-c]pyridin-5(4H)-yl)(phenyl)methanone;
1-(2-(3,8-diazabicyclo[3.2.1]octan-8-yl)-7,7-difluoro-6,7-dihydrothiazolo[5,4-c]pyridin-5(4H)-yl)-2-cyclopentylethan-1-one;
(S)-2-cyclopentyl-1-(2-(hexahydropyrrolo[1,2-a]pyrazin-2 (1H)-yl)-6,7-dihydrothiazolo[5,4-c]pyridin-5(4H)-yl)ethan-1-one;
(R)-2-cyclopentyl-1-(2-(hexahydropyrrolo[1,2-a]pyrazin-2 (1H)-yl)-6,7-dihydrothiazolo[5,4-c]pyridin-5(4H)-yl)ethan-1-one;
1-(2-(3,9-diazabicyclo[3.3.1]nonan-9-yl)-6,7-dihydrothiazolo[5,4-c]pyridin-5(4H)-yl)-2-cyclopentylethan-1-one;
3,3-dimethyl-1-(2-(piperazin-1-yl)-6,7-dihydrothiazolo[5,4-c]pyridin-5(4H)-yl) butan-1-one;
(R)-2-cyclopentyl-1-(2-(2-(hydroxymethyl)piperazin-1-yl)-6,7-dihydrothiazolo[5,4-c]pyridin-5(4H)-yl)ethan-1-one;
1-(2-(3,8-diazabicyclo[3.2.1]octan-3-yl)-6,7-dihydrothiazolo[5,4-c]pyridin-5(4H)-yl)-2-cyclopentylethan-1-one;
1-(2-((1 S,4S)-2,5-diazabicyclo[2.2.1]heptan-2-yl)-6,7-dihydrothiazolo[5,4-c]pyridin-5(4H)-yl)-2-cyclopentylethan-1-one;
1-(2-(2,5-diazabicyclo[2.2.2]octan-2-yl)-6,7-dihydrothiazolo[5,4-c]pyridin-5(4H)-yl)-2-cyclopentylethan-1-one;
1-(2-((1R,4R)-2,5-diazabicyclo[2.2.1]heptan-2-yl)-6,7-dihydrothiazolo[5,4-c]pyridin-5(4H)-yl)-2-cyclopentylethan-1-one;
1-(2-(3,9-diazabicyclo[3.3.1]nonan-3-yl)-6,7-dihydrothiazolo[5,4-c]pyridin-5(4H)-yl)-2-cyclopentylethan-1-one;
1-(2-(3,8-diazabicyclo[3.2.1]octan-8-yl)-6,7-dihydrothiazolo[4,5-c]pyridin-5(4H)-yl)-2-cyclopentylethan-1-one;
1-(2-(3,8-diazabicyclo[3.2.1]octan-8-yl)-6,7-dihydrothiazolo[4,5-c]pyridin-5(4H)-yl)-3,3-dimethylbutan-1-one;
2-cyclopentyl-1-(2-(7-hydroxy-3,9-diazabicyclo[3.3.1]nonan-9-yl)-6,7-dihydrothiazolo[5,4-c]pyridin-5(4H)-yl)ethan-1-one;
(S)-2-cyclopentyl-1-(2-(2-(hydroxymethyl)piperazin-1-yl)-6,7-dihydrothiazolo[5,4-c]pyridin-5(4H)-yl)ethan-1-one;
2-(3,8-diazabicyclo[3.2.1]octan-8-yl)-5-((4-fluorobenzyl) sulfonyl)-4,5,6,7-tetrahydrothiazolo[5,4-c]pyridine;
2-(3-ethyl-3,8-diazabicyclo[3.2.1]octan-8-yl)-5-((4-fluorobenzyl) sulfonyl)-4,5,6,7-tetrahydrothiazolo[5,4-c]pyridine;
5-((4-fluorobenzyl) sulfonyl)-2-(piperazin-1-yl)-4,5,6,7-tetrahydrothiazolo[5,4-c]pyridine;
5-(benzylsulfonyl)-2-(3,8-diazabicyclo[3.2.1]octan-8-yl)-4,5,6,7-tetrahydrothiazolo[5,4-c]pyridine;
5-(benzylsulfonyl)-2-(3-ethyl-3,8-diazabicyclo[3.2.1]octan-8-yl)-4,5,6,7-tetrahydrothiazolo[5,4-c]pyridine;
2-cyclopentyl-1-(2-(3-isopropyl-3,8-diazabicyclo[3.2.1]octan-8-yl)-6,7-dihydrothiazolo[5,4-c]pyridin-5(4H)-yl)ethan-1-one;
2-cyclopentyl-1-(2-(3-ethyl-3,8-diazabicyclo[3.2.1]octan-8-yl)-6,7-dihydrothiazolo[5,4-c]pyridin-5(4H)-yl)ethan-1-one;
2-cyclopentyl-1-(2-(3-propyl-3,8-diazabicyclo[3.2.1]octan-8-yl)-6,7-dihydrothiazolo[5,4-c]pyridin-5(4H)-yl)ethan-1-one;
2-(3,8-diazabicyclo[3.2.1]octan-8-yl)-N—((S)-chroman-4-yl)-6,7-dihydrothiazolo[5,4-c]pyridine-5(4H)-carboxamide;
N—((S)-chroman-4-yl)-2-(3-ethyl-3,8-diazabicyclo[3.2.1]octan-8-yl)-6,7-dihydrothiazolo[5,4-c]pyridine-5(4H)-carboxamide;
N—((R)-chroman-4-yl)-2-(3-ethyl-3,8-diazabicyclo[3.2.1]octan-8-yl)-6,7-dihydrothiazolo[5,4-c]pyridine-5(4H)-carboxamide;
(S)—N-(chroman-4-yl)-2-(piperazin-1-yl)-6,7-dihydrothiazolo[5,4-c]pyridine-5(4H)-carboxamide;
(S)—N-(chroman-4-yl)-2-(4-ethylpiperazin-1-yl)-6,7-dihydrothiazolo[5,4-c]pyridine-5(4H)-carboxamide;
2-(4-fluorophenoxy)-1-(2-(3-isopropyl-3,8-diazabicyclo[3.2.1]octan-8-yl)-6,7-dihydrothiazolo[5,4-c]pyridin-5(4H)-yl)ethan-1-one;
1-(2-(4-ethylpiperazin-1-yl)-6,7-dihydrothiazolo[5,4-c]pyridin-5(4H)-yl)-3,3-dimethylbutan-1-one;
2-(3-ethyl-3,8-diazabicyclo[3.2.1]octan-8-yl)-N-(4-fluorobenzyl)-6,7-dihydrothiazolo[5,4-c]pyridine-5(4H)-carboxamide;
2-(4-fluorophenoxy)-1-(2-(3-methyl-3,8-diazabicyclo[3.2.1]octan-8-yl)-6,7-dihydrothiazolo[5,4-c]pyridin-5(4H)-yl)ethan-1-one;
1-(2-(3-ethyl-3,8-diazabicyclo[3.2.1]octan-8-yl)-6,7-dihydrothiazolo[5,4-c]pyridin-5(4H)-yl)-2-(4-fluorophenoxy) ethan-1-one;
2-(4-fluorophenoxy)-1-(2-(3-propyl-3,8-diazabicyclo[3.2.1]octan-8-yl)-6,7-dihydrothiazolo[5,4-c]pyridin-5(4H)-yl)ethan-1-one;
2-(4-ethylpiperazin-1-yl)-5-((4-fluorobenzyl) sulfonyl)-4,5,6,7-tetrahydrothiazolo[5,4-c]pyridine;
2-cyclopentyl-1-(2-(6-methylpyridin-3-yl)-6,7-dihydrothiazolo[5,4-c]pyridin-5(4H)-yl)ethan-1-one;
2-cyclopentyl-1-(2-(5-methylpyridin-3-yl)-6,7-dihydrothiazolo[5,4-c]pyridin-5(4H)-yl)ethan-1-one;
   and pharmaceutically acceptable salts thereof.

In a further embodiment of a compound of Formula I or Ia, R¹ may be as defined in any of the compounds of Formula I or Ia, above. In a further embodiment of a compound of Formula I or Ia, A may be as defined in any of the compounds of Formula I or Ia, above.

Further aspects and embodiments are as set out in the following numbered clauses.

Clause 1. A compound of Formula Ia

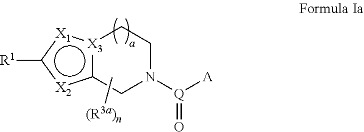

or a pharmaceutically acceptable salt or derivative thereof, wherein:
one of X₁ and X₂ is N and the other is N, S or CR^{3b}, X₃ is N or C;
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1;
   for use in the treatment or prevention of a disease or disorder that can be ameliorated by activation of long isoforms of PDE4.
   Clause 2. The compound for use of Clause 1, where in the compound is a compound of formula:

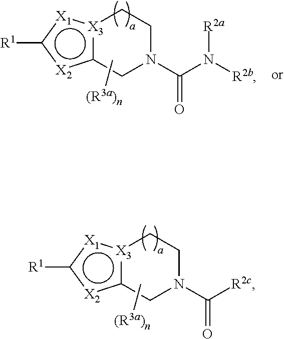

or a pharmaceutically acceptable salt or derivative thereof.
Clause 3. The compound, or a pharmaceutically acceptable salt or derivative thereof, for use of Clause 1 or 2, wherein R¹ is a 5- to 6-membered saturated, monocyclic ring containing at least 1 ring N heteroatom and optionally a ring O heteroatom; a 6-membered aromatic, monocyclic ring containing 1 or 2 ring N heteroatoms; a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.
Clause 4. The compound, or a pharmaceutically acceptable salt or derivative thereof, for use of any preceding Clause, wherein R¹ is a 4- to 10-membered non-aromatic ring that may be monocyclic, bridged or bicyclic containing at least 1 ring N heteroatom and optionally a ring O heteroatom; wherein R¹ is optionally substituted with 1 R⁴.
Clause 5. The compound, or a pharmaceutically acceptable salt or derivative thereof, for use of any one of Clause 1 to 3, wherein R¹ is a 7- to 9-membered saturated, bridged ring system containing 2 ring N heteroatoms, optionally a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.
Clause 6. The compound, or a pharmaceutically acceptable salt or derivative thereof, for use of any preceding Clause, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵; or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵.
   Clause 7. The compound, or a pharmaceutically acceptable salt or derivative thereof, for use of any preceding Clause, wherein:
a) R^{2a} is a (C5-10)alkyl group comprising a cyclic moiety; and wherein R^{2a} is optionally substituted with 1 or more R⁵; and R^{2b} is H or (C1-6)alkyl, and wherein (C1-6)alkyl is optionally substituted with 1 or more R⁵; or
b) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2a} is substituted with 1 or more R⁵ (optionally wherein R⁵ is halogen); and R^{2b} is H or (C1-6)alkyl, and wherein (C1-6)alkyl is optionally substituted with 1 or more R⁵.
   Clause 8. The compound, or a pharmaceutically acceptable salt or derivative thereof, for use of any preceding Clause, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is substituted with 1 or more R⁵ (optionally wherein R⁵ is halogen).
   Clause 9. The compound, or a pharmaceutically acceptable salt or derivative thereof, for use of any one of Clause 1 to 7, wherein the compound is of formula:

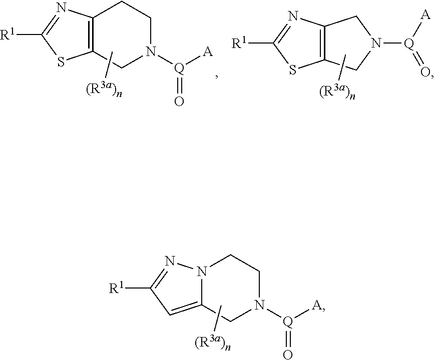

or a pharmaceutically acceptable salt or derivative thereof.
Clause 10. The compound, or a pharmaceutically acceptable salt or derivative thereof, for use of any preceding Clause, wherein each R^{3a} is-CH₃ or F, or two R^{3a} attached to the same carbon are joined together with the atom to which they are attached to form a cyclopropyl ring.
Clause 11. The compound, or a pharmaceutically acceptable salt or derivative thereof, for use of any preceding Clause, wherein n is 0, 1 or 2.
Clause 12. The compound, or a pharmaceutically acceptable salt or derivative thereof, for use of any preceding Clause, wherein the compound is a compound of Formula Ia, one of X₁ and X₂ is N and the other is S, and X₃ is C.
Clause 13. The compound, or a pharmaceutically acceptable salt or derivative thereof, for use of any preceding Clause, wherein Q is C and/or A is R^{2c}.
Clause 14. The compound, or a pharmaceutically acceptable salt or derivative thereof, for use of any preceding Clause, wherein
Q is C;
R¹ is a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.
   Clause 15. A compound of Formula IIa:

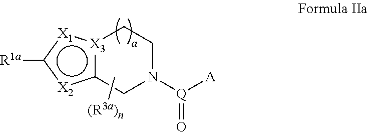

or a pharmaceutically acceptable salt or derivative thereof, wherein:
one of X₁ and X₂ is N and the other is N, S or CR^{3b}, X₃ is N or C; Q is C or S(O);
R^{1a} is a 7- to 9-membered saturated, bridged ring system containing 2 ring N heteroatoms, and wherein R^{1a} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.
   Clause 16. The compound, or a pharmaceutically acceptable salt or derivative thereof, of Clause 15, wherein R^{1a} is a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.
   Clause 17. The compound, or a pharmaceutically acceptable salt or derivative thereof, of any Clause 15 or 16, wherein the compound is of formula:

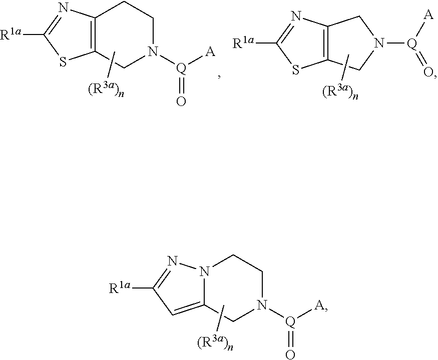

or a pharmaceutically acceptable salt or derivative thereof.
Clause 18. The compound, or a pharmaceutically acceptable salt or derivative thereof, of any of Clause 15-17, wherein the compound is a compound of Formula IIa, one of X₁ and X₂ is N and the other is S and X₃ is C.
Clause 19. A compound of Formula IIIa:

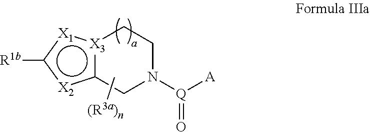

or a pharmaceutically acceptable salt or derivative thereof, wherein:
one of X₁ and X₂ is N and the other is N, S or CR^{3b}, X₃ is N or C;
Q is C or S(O);
R^{1b} is a 4- to 10-membered non-aromatic ring that may monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered ring, optionally additionally containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1;
wherein when one of X₁ and X₂ is N and the other is S and X₃ is C, R^{1b} is not a monocyclic piperazine ring.
   Clause 20. The compound or a pharmaceutically acceptable salt or derivative thereof of Clause 19, wherein R^{1b} is a 5- to 6-membered saturated, monocyclic ring containing at least 1 ring N heteroatom and optionally a ring O heteroatom; or a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R^{1b} is optionally substituted with 1, 2 or 3 R⁴.
   Clause 21. The compound, or a pharmaceutically acceptable salt or derivative thereof, of Clause 19 or 20, wherein R^{1b} is a 6-membered saturated monocyclic ring containing 2 ring N heteroatoms or a 7- to 9-membered saturated, bridged ring system containing 2 ring N heteroatoms; wherein R^{1a} is optionally substituted with 1 R⁴, optionally wherein R^{1a} is a 7- to 8-membered saturated, bridged ring system containing 2 ring N heteroatoms (for example a bridged piperazine, such as 3,8-diazabicyclo[3.2.1]octanyl), wherein R^{1b} is optionally substituted with 1 R⁴.
   Clause 22. The compound, or a pharmaceutically acceptable salt or derivative thereof, of any of Clause 15-21, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵; and/or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵.
   Clause 23. The compound, or a pharmaceutically acceptable salt or derivative thereof, of any of Clause 15-22, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is substituted with 1 or more R⁵ (optionally wherein R⁵ is halogen).
   Clause 24. The compound, or a pharmaceutically acceptable salt or derivative thereof, of any of Clause 15-23, wherein the compound is of formula:

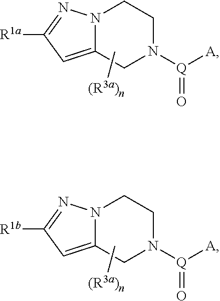

or a pharmaceutically acceptable salt or derivative thereof.
Clause 25. The compound, or a pharmaceutically acceptable salt or derivative thereof, of any of Clause 15-24, wherein Q is C and/or A is R^{2c}.
Clause 26. The compound, or a pharmaceutically acceptable salt or derivative thereof, of any of Clause 15-24, wherein
Q is C;
R^{1a} or R^{1b} is a 7- to 9-membered saturated, bridged ring system containing 2 ring N heteroatoms, wherein R^{1a} and R^{1b} are optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.
   Clause 27. A compound of Formula IVa:

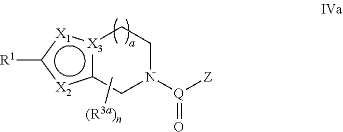

or a pharmaceutically acceptable salt or derivative thereof, wherein:
one of X₁ and X₂ is N and the other is N, S or CR^{3b}, X₃ is N or C;
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
Z is NR^{2d}R^{2b} or R^{2e};
R^{2b} is H or (C1-6)alkyl, and wherein (C1-6)alkyl is optionally substituted with 1 or more R⁵; and
R^{2d} is
   a) a (C5-10)alkyl group comprising a cyclic moeity; and wherein R^{2d} is optionally substituted with 1 or more R⁵; or
   b) a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2d} is substituted with 1 or more R⁵ (optionally wherein R⁵ is halogen);
R^{2e} is
   a) a (C3-10)alkyl group comprising a cyclic moeity, wherein a C atom in straight chain or cyclic portion of said (C3-10)alkyl group may be optionally replaced by 1 —O— other than at the point of attachment of R^{2e}, wherein said (C3-10)alkyl group may be substituted with a 6-membered aromatic or heteroaromatic ring that contains 0, 1 or 2 ring N atoms; and wherein R^{2e} is optionally substituted with 1 or more R⁵; or
   b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2e}, wherein said (C3-10)alkyl group maybe substituted with a 6-membered aromatic or heteroaromatic ring that contains 0, 1 or 2 ring N atoms; and wherein R^{2e} is substituted with 1 or more R⁵ (optionally wherein R⁵ is halogen);
each R^{3a} is independently (C1-6)alkyl, optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
n is 0, 1, 2, 3 or 4; and
a is 0 or 1.
   Clause 28. The compound, or a pharmaceutically acceptable salt or derivative thereof, of Clause 27, wherein Z is R^{2e}.
   Clause 29. The compound or a pharmaceutically acceptable salt or derivative thereof, of Clause 27 or 28, wherein R¹ is a 5- to 6-membered saturated, monocyclic ring containing at least 1 ring N heteroatom and optionally a ring O heteroatom; a 6-membered aromatic, monocyclic ring containing 1 or 2 ring N heteroatoms; a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.
   Clause 30. The compound, or a pharmaceutically acceptable salt or derivative thereof, of any of Clause 27-29, wherein R¹ is a 4- to 10-membered non-aromatic ring that may be monocyclic, bridged or bicyclic containing at least 1 ring N heteroatom and optionally a ring O heteroatom; wherein R¹ is optionally substituted with 1 R⁴.
   Clause 31. The compound, or a pharmaceutically acceptable salt or derivative thereof, of any of Clause 27-30, wherein R¹ is a 7- to 9-membered saturated, bridged ring system containing 2 ring N heteroatoms (for example a bridged piperazine, such as 3,8-diazabicyclo[3.2.1]octanyl), wherein R¹ is optionally substituted with 1 R⁴.
   Clause 32. The compound, or a pharmaceutically acceptable salt or derivative thereof, of any of Clause 15-31, wherein each R^{3a} is —CH₃, or two R^{3a} attached to the same carbon are joined together with the atoms to which they are attached to form a cyclopropyl ring.
   Clause 33. The compound, or a pharmaceutically acceptable salt or derivative thereof, of any of Clause 15-32, wherein n is 0, 1 or 2.
   Clause 34. A compound of Formula Va:

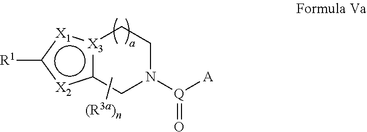

or a pharmaceutically acceptable salt or derivative thereof, wherein:
one of X₁ and X₂ is N and the other is N, S or CR^{3b}, X₃ is N or C;
Q is C or S(O);
R¹ is a 4- to 10-membered monocyclic, bridged or bicyclic ring containing at least 1 ring N heteroatom and optionally a ring O heteroatom, and wherein R¹ is optionally substituted with 1 or more R⁴;
A is NR^{2a}R^{2b} or R^{2c};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 7-membered non-aromatic heterocycle, optionally containing 1 further heteroatom selected from O, and optionally substituted with 1 or more R⁵;
R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered ring, optionally additionally containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
m is 1, 2, 3 or 4; and
a is 0 or 1.

### Definitions

The term “aromatic ring” refers to an aromatic carbocyclic ring system. The term “heteroaromatic ring” refers to an aromatic ring system wherein one or more of the ring-forming atoms is a heteroatom such as O, S or N. An aromatic ring may be a 6-membered aromatic ring, i.e. a phenyl ring. A heteroaromatic ring may be a 6-membered heteroaromatic ring that contains one to three N atoms or a 5-membered heteroaromatic ring that contains one to three heteroatoms selected from O, S and N. Examples of such 6- or 5-membered heteroaromatic rings include pyridine, pyridazine, pyrazine, pyrimidine, thiophene, furan, thiazole, thiadiazole, oxazole, oxadiazole, imidazole, triazole and their isomers including isothiazole, isothiadiazole, isoxazole and isoxadiazole. In all instances described above, an aromatic ring may be optionally substituted as defined herein.

The term “carbocyclic ring” refers to a ring system with may be saturated, partially unsaturated or aromatic and wherein all ring forming atoms are carbon. The term “heterocyclic ring” refers to a ring system with may be saturated, partially unsaturated or aromatic and wherein one or more of the ring-forming atoms is a heteroatom such as O, S or N. A “non-aromatic carbocyclic or heterocyclic ring” may be saturated or partially unsaturated. Carbocyclic and heterocyclic rings may be bicyclic or multicyclic ring systems, for example bicyclic or multicyclic fused ring systems or bicyclic or multicyclic spiro ring systems or a combination thereof. Each ring within a fused ring system may independently be saturated, partially unsaturated or aromatic. Examples of such fused bicyclic ring systems include indane and chromane. A non-aromatic carbocyclic or heterocyclic ring may include fused ring systems, where for example two rings share two adjacent atoms, bridged ring systems, where for example two rings share three or more adjacent atoms, or spiro ring systems, where for example two rings share one adjacent atom. Examples of fused ring systems include octahydropyrrolo[1,2-a]pyrazine and octahydro-2H-pyrido[1,2-a]pyrazine. Bridged rings may comprise three or more rings. Examples of such bridged ring systems include 2,5-diazabicyclo[2.2.1]heptane, 2,5-diazabicyclo[2.2.2]octane and 3,8-diazabicyclo[3.2.1]octane. Examples of spiro ring systems include spiro[4.3]octane and 2,6-diazaspiro[3.4]octane. In all instances described above, a carbocyclic or heterocyclic ring may be optionally substituted as defined herein.

Where a ring is referred to herein as containing specified ring heteroatoms, it will be appreciated that no further ring heteroatoms are present beyond those specified.

A “monocyclic, bridged or bicyclic ring” includes monocyclic rings, bridged ring systems and bicyclic ring systems. A “monocyclic, bridged or bicyclic ring”, unless otherwise defined, may be saturated, partially unsaturated or aromatic. These may be aromatic, heteroaromatic, carbocyclic or heterocyclic rings or combinations thereof. Bicyclic ring systems may include fused and spiro rings.

Unless otherwise defined, the term “alkyl” as used herein refers to a saturated hydrocarbon which may be straight-chain, branched, cyclic or a combination thereof. Alkyl groups include linear, branched or cyclic alkyl groups and hybrids thereof, such as (cycloalkyl)alkyl. The term “(C1-6)alkyl” as used herein means an alkyl group having 1-6 carbon atoms, which may be branched or unbranched and optionally contains a ring. Examples of (C1-6)alkyl include hexyl, cyclohexyl, pentyl, cyclopentyl, butyl, isobutyl, cyclobutyl, tertiary butyl, propyl, isopropyl, cyclopropyl, cyclopropylmethyl, ethyl and methyl. The term “(C1-4)alkyl” as used herein means a branched or unbranched alkyl group having 1-4 carbon atoms, optionally containing a ring. Examples of (C1-4)alkyl include butyl, isobutyl, cyclobutyl, tertiary butyl, propyl, isopropyl, cyclopropyl, cyclopropylmethyl, ethyl and methyl. A (C1-4)alkyl as referenced herein may preferably be a (C1-2)alkyl. Where specified in the formulae above, (C1-4)alkyl may be substituted, for example with 1 to 3 fluoros. A particularly preferred example of a substituted (C1-4)alkyl is trifluoromethyl. Alternatively (C1-4)alkyl may be unsubstituted.

The term “alkylene” as used herein refers to a divalent alkyl group.

The term “cycloalkyl” refers to a cyclic alkyl group, for example cycloheptyl, cyclohexyl, cyclopentyl, cyclobutyl or cyclopropyl. Cycloalkyl may be substituted as defined herein.

The term “alkoxy” means-O-alkyl wherein alkyl has the meaning as defined above. Examples of (C1-4)alkoxy include methoxy, ethoxy, propoxy, isopropoxy, butoxy, isobutoxy and tertiary butoxy. A (C1-4)alkoxy as referenced herein may preferably be a (C1-2)alkoxy. Where specified in the formulae above, (C1-4)alkoxy may be substituted, for example with 1 to 3 fluoros. A particularly preferred example of a substituted (C1-4)alkoxy is trifluoromethoxy. Alternatively, (C1-4)alkoxy may be unsubstituted. In the present invention, alkoxy is attached to the rest of the molecule by the “oxy” moiety.

A group that is referred to herein as being “substituted”, whether preceded by the term “optionally” or not, means that at least one hydrogen present on a group (e.g. a C or N atom) is replaced with a permissible substituent, for example a substituent which upon substitution results in a stable compound, e.g. a compound which does not spontaneously undergo transformation such as by rearrangement, cyclization, elimination or other reaction. Unless otherwise indicated, when more than one substituent is present, the substituent is either the same or different at each occurrence. Unless otherwise indicated, a “substituted” group has one or more substituents at one or more substitutable positions of the group, and when more than one position in any given structure is substituted, the substituent is either the same or different at each position.

The term “may” as used herein is interpreted as being optional. Where a feature is referred to as “may be” present, said feature is optionally present. For example, where a moiety is described as “may be substituted”, said moiety is optionally substituted, i.e. it is either unsubstituted or is substituted as described.

The term “halogen” means F, Cl, Br or I. F and Cl are particularly preferred, with F the most preferred.

### Activation of Long PDE4 Isoforms

PDE4 long isoforms have two regulatory regions, upstream conserved region 1 (UCR1) and upstream conserved region 2 (UCR2). These are between the isoform-specific N-terminal portion and the catalytic domain. The UCR1 domain is missing in the short forms, whereas the super-short forms not only lack UCR1, but also have a N-terminal truncated UCR2 domain (Houslay, M. D., Schafer, P. and Zhang, K. Drug Discovery Today 10: 1503-1519, 2005).

There are four PDE4 families, PDE4A, PDE4B, PDE4C and PDE4D. The present invention concerns compounds that are capable of activating one or more of the long isoforms from one or more of these four families. The long isoform PDE4 may therefore be long isoform PDE4A, long isoform PDE4B, long isoform PDE4C or long isoform PDE4D. For the avoidance of doubt, a long isoform PDE4 contains a UCR1 region. In some embodiments, a long isoform PDE4 as referred to herein is human. UCR1 is conserved within mammalian species (Houslay, M D, Sullivan, M and Bolger G B Adv. Pharmacol. 44: 225-342, 1998), so in other embodiments, the long isoform PDE4 can be from a non-human mammal.

Without wishing to be bound by theory, the compounds described herein may act as PDE4 long form activators. The compounds described herein are small molecules that are believed to bind directly to PDE4 long forms and induce structural changes that increase, stabilise, uncover and/or maintain the catalytic activity of these enzymes. Without wishing to be bound by theory, the activation of PDE4 long forms by PDE4 long form activators may be sensitive to the regulatory status of the enzyme, including post-translational modifications (such as phosphorylation) or the adoption of protein-protein complexes associated with a particular physiological localisation or with a cellular or biochemical assay context. PDE4 long form activators may manifest activation of the enzyme in one or more states but not necessarily all states.

In the field of pharmacology, and as used herein, a small molecule is defined as a low molecular weight organic compound that may serve as a regulator of biological processes. Preferred small molecule activators according to the present invention have a molecular weight of less than or equal to 700 Daltons. This allows for the possibility to rapidly diffuse across cell membranes and reach intracellular sites of action (Veber, D. F. et al., J. Med. Chem. 45: 2615-2623, 2002). Especially preferred small molecule activators according to the present invention have molecular weights of greater than or equal to 250 Daltons and less than or equal to 500 Daltons (Lipinski, C. A. Drug Discovery Today: Technologies 1: 337-341, 2004).

One suitable method of detecting whether or not a compound is capable of serving as an activator of a PDE4 long form is using a two-step radio-assay procedure described in Experiment 1. In summary, the method involves incubating a PDE4 long form with a test small molecule activator, together with [³H]-labelled cAMP to assess alterations in the breakdown of cAMP to the 5′-adenosine monophosphate (5′-AMP) product. A sample of the reaction mixture from such an incubation is subsequently treated with snake venom 5′-nucleotidase to allow conversion of the nucleotide [³H]-labelled 5′-AMP to the uncharged nucleoside [³H]-labelled adenosine, which can be separated and quantified to assess PDE4 activity and the effect of the test compound (Thompson, W. J. and Appleman, M. M. Biochemistry 10: 311-316, 1971, with some modifications as described in: Marchmont, R. J. and Houslay, M. D. Biochem J. 187: 381-92, 1980).

Using the above assay procedure, as described in detail in Experiment 1, preferred compounds described herein may produce an increase in the background activity of one or more PDE4 long forms of more than 20% or more than 30% at a test compound concentration of 100 micromolar or less. Especially preferred compounds described herein may produce an increase in the background activity of one or more PDE4 long forms of more than 20% or more than 30% at a test compound concentration of 10 micromolar, or less, for example 3 micromolar.

The compounds described herein may be selective for the long form of the PDE4 enzyme and, as such, do not act or act to a lesser extent as activators of the short or super-short isoforms of the PDE4 enzyme. The short or super-short isoform PDE4 may be short or super-short isoform PDE4A, short or super-short isoform PDE4B, short or super-short isoform PDE4C, or short or super-short isoform PDE4D. For the avoidance of doubt, short and super-short isoforms of PDE4 lack a UCR1 domain. Super-short isoforms are characterised by a truncated UCR2 domain and lack of a UCR1 domain. The short or super-short isoform PDE4 is, for example, human, but may also be from other mammalian species (where UCR2 is conserved, see Houslay, M D, Sullivan, M and Bolger GB Adv. Pharmacol. 44:225-342, 1998).

Under the same assay conditions, as described in Experiment 1, the compounds described herein may produce a less than 30% or less than 20% increase in the background activity of the short or super-short forms of the PDE4A, PDE4B, PDE4C or PDE4D enzymes at a test compound concentration of 100 micromolar, or less.

Compounds described herein may therefore provide a positive result in an assay for activation of a long form PDE4 and a negative result in an assay for activation of a short form (or super-short form) of PDE4.

PDE4 long isoforms include those now known as PDE4A4, PDE4A4/5, PDE4A5, PDE4A8, PDE4A10, PDE4A11, PDE4B1, PDE4B3, PDE4B4, PDE4C1, PDE4C2, PDE4C3, PDE4C4, PDE4D3, PDE4D4, PDE4D5, PDE4D7, PDE4D8, PDE4D9 and PDE4D11. Further long isoforms may be or have already been identified or called by different nomenclature from any of the four PDE4 sub-families.

PDE4 short and super-short isoforms include PDE4A1, PDE4B2, PDE4B5, PDE4D1, PDE4D2, PDE4D6 and PDE4D10. Further short and super-short isoforms may be or have already been identified or called by different nomenclature from any of the four PDE4 sub-families.

The Examples below exemplify activity of compounds described herein in an assay for activation of the human PDE4D5 long isoforms and a lack of activity in an assay for activation of the human PDE4B2 short isoform. Details of these isoforms and a number of the other known isoforms, including GenBank accession numbers, are provided in Tables A to D immediately below.

TABLE A
Examples of known PDE4A Isoforms

| | | | Calculated | |
|---|---|---|---|---|
| | | | molecular | |
| Isoform | Species | Accession | weight (kDa) | Type |
| PDE4A1 | Human | NM_006202 | 73 | Short |
| PDE4A1 | Rodent | L27062 | 68 | Short |
| PDE4A4* | Human | L20965 | 98 | Long |
| PDE4A5 | Rodent | L27057 | 93 | Long |
| PDE4A7** | Human | U18088 | 37 | Dead-Short |
| PDE4A8 | Human | AY593872 | 96 | Long |
| PDE4A8 | Rodent | L36467 | 85 | Long |
| PDE4A10 | Human | AF073745 | 91 | Long |
| PDE4A11 | Human | AY618547 | 95 | Long |

*Note that the PDE4A4B clone is correct while PDE4A4A has a cloning artefact and PDE4A4C is a truncation artefact.
**Note that this species is C- as well as N-terminally truncated

TABLE B
Examples of known PDE4B Isoforms

| | | | Calculated | |
|---|---|---|---|---|
| | | | molecular | |
| Isoform | Species | Accession | weight (kDa) | Type |
| PDE4B1 | Human | NM_001037341.1 | 83 | Long |
| PDE4B2 | Human | NM_001037339.1 | 64 | Short |
| PDE4B3 | Human | NM_001037340 | 83 | Long |
| PDE4B4 | Rodent | AF202733.1 | 75 | Long |
| PDE4B5 | Human | EF595686.1 | 57 | Super-short |

TABLE C
Examples of known PDE4C Isoforms

| | | | Calculated | |
|---|---|---|---|---|
| | | | molecular | |
| Isoform | Species | Accession | weight (kDa) | Type |
| PDE4C1 | Human | NM_000923 | 79 | Long |
| PDE4C2 | Human | NM_001098819 | 67 | Long |
| PDE4C3 | Human | NM_001098818 | 76 | Long |
| PDE4C4 | Human | U66346 | 88 | Long |
| PDE4C5 | Human | U66347 | 47 | Partial |
| PDE4C6 | Human | U66348 | 58 | Partial |
| PDE4C7 | Human | U66349 | 48 | Partial |

TABLE D
Examples of known PDE4D Isoforms

| | | | Calculated | |
|---|---|---|---|---|
| | | | molecular | |
| Isoform | Species | Accession | weight (kDa) | Type |
| PDE4D1 | Human | NM_001197222 | 66 | Short |
| PDE4D2 | Human | NM_001197221 | 58 | Super-short |
| PDE4D3 | Human | NM_006203 | 76 | Long |
| PDE4D4 | Human | NM_001104631 | 91 | Long |
| PDE4D5 | Human | NM_001197218 | 84 | Long |
| PDE4D6 | Human | NM_001197223 | 59 | Super-short |
| PDE4D7 | Human | NM_001165899 | 85 | Long |
| PDE4D8 | Human | NM_001197219 | 78 | Long |
| PDE4D9 | Human | NM_001197220 | 77 | Long |
| PDE4D10 | Rodent | DQ665896.1 | 58 | Super-short |
| PDE4D11 | Rodent | EU489880.1 | 79 | Long |

*PDE4D8 was originally called PDE4D6 in the literature

Reduction of cAMP Levels

Without wishing to be bound by theory, the compounds described herein may function by reducing cAMP levels in one or more intracellular compartments. The PDE4 long form activators described herein may thus provide a means to regulate certain cellular processes that are dependent upon cAMP. Excessive intracellular cAMP signalling mediates a number of diseases and disorders. Therefore, the compounds described herein are expected to be of utility for the treatment of diseases associated with abnormally elevated cAMP levels, increased cAMP-mediated signalling and/or reduced cAMP elimination, enzymatic or otherwise (e.g. via efflux). The treatment is typically of a human, but may also be of a non-human animal, such as a non-human mammal (e.g. veterinary treatment).

In one aspect, the present invention provides a compound described here (i.e. a small molecule activator of a PDE4 long form), for use in a method for the treatment or prevention of disorders where a reduction of second messenger responses mediated by cyclic 3′,5′-adenosine monophosphate (cAMP) is required.

For example, gain-of-function gene mutations in proteins involved in driving cAMP signalling upstream of adenylyl cyclase, such as GPCRs and Gsα, can lead to abnormal excessive cAMP activity with pathological consequences (Lania A, Mantovani G, Spada A. Ann Endocrinol (Paris). 73: 73-75, 2012.; Thompson, M. D. et al., Methods Mol. Biol. 448: 109-137, 2008; Weinstein L S, Liu J, Sakamoto A, Xie T, Chen M. Endocrinology. 145: 5459-5464, 2004; Lania A, Mantovani G, Spada A. Eur J Endocrinol. 145: 543-559, 2001). PDE4 long form activators described herein, possessing the ability to accelerate the termination of cAMP action, would therefore be expected to be effective in the treatment, prevention or partial control of diseases characterised by undesirably high cAMP levels, or activity, as detailed below.

The treatment or prevention described herein may be treatment or prevention of a disease or disorder that can be ameliorated by activation of long isoforms of PDE4. The treatment or prevention described herein may be treatment or prevention of a disease or disorder mediated by excessive intracellular cAMP signalling. In these diseases, a reduction of second messenger responses mediated by cyclic 3′,5′-adenosine monophosphate (cAMP) should provide a therapeutic benefit.

Diseases Ameliorated by Activation of Long Isoforms of PDE4 or Characterised by Elevated cAMP Levels

### Hyperthyroidism

Stimulation of the thyroid-stimulating hormone (TSH) receptor (TSHR) leads to increased generation and release of thyroid hormones, thyroxine and triiodothyronine, through a cAMP-dependent signalling mechanism involving Gsα-mediated activation of adenylyl cyclase. Gain-of-function mutations in the TSHR have been reported to be involved in the development of hyperthyroidism (Duprez, L. et al., Nat. Genet. 7: 396-401, 1994; Biebermann, H. et al., J. Clin. Endocrinol. Metab. 86: 4429-4433, 2001; Karges, B. et al., J. Endocrinol. 186: 377-385, 2005). Activating mutations of both TSHR and Gsα have also been found in goitre and thyroid adenomas (Arturi, F. et al., Exp. Clin. Endocrinol. Diabetes 106: 234-236, 1998). The increased cAMP activity in thyroid adenomas, as a result of the activating TSHR or Gsα mutations, has been reported to produce a protective adaptive increase in PDE4 activity to counteract abnormal rise in cAMP levels and signal transduction (Persani, L. et al., J. Clin. Endocrinol. Metab. 85:2872-2878, 2000).

The most common cause of hyperthyroidism is Graves' disease, an autoimmune disorder in which antibodies mimic TSH action at the TSHR, leading to excessive cAMP activity in thyroid follicle cells and consequently a state of hyperthyroidism.

PDE4 long form activators described herein are therefore expected to be effective in the treatment, prevention or partial control of hyperthyroidism. In one embodiment, the hyperthyroidism is associated with Graves' disease.

### Jansens's Metaphyseal Chondrodysplasia

Jansens's Metaphyseal Chondrodysplasia (JMC) is a very rare disease resulting from gain-of-function mutations of the parathyroid hormone (PTH) receptor 1 (PTHR1) (Thompson, M. D. et al., Methods Mol. Biol. 448:109-137, 2008). The constitutive activation of the PTHR1 which couples to adenylyl cyclase as effector is associated with excessive cAMP signalling primarily in bone and kidney, leading to dysregulation of ion homeostasis characterised by hypercalcemia and hypophosphatemia (Calvi, L. M. and Schipani, E. J. Endocrinol. Invest. 23: 545-554, 2000) and developmental (e.g. short stature) and physical (e.g. protruding eyes) abnormalities. PDE4 long form activators described herein are therefore expected to be effective in the treatment, prevention or partial control of JMC.

### Hyperparathyroidism

Hyperparathyroidism (HPT) is characterized by excessive secretion from the parathyroid gland of PTH, which regulates plasma calcium and phosphate concentrations via PTHR1 receptors in the kidney, bone and GI tract. The resulting excessive stimulation of these receptors causes disruption of plasma ion homeostasis with patients showing hypercalcemia and hypophosphatemia. Primary HPT is driven by parathyroid gland hyperplasia or dysfunction, whereas secondary HPT is associated with underlying medical conditions, predominantly chronic renal disease. Left untreated, HPT causes a variety of debilitating symptoms and can become life-threatening.

By acting to down-regulate excessive cAMP generated by sustained PTH signalling, PDE4 long form activators described herein are expected to be effective in the treatment, prevention or partial control of hyperparathyroidism.

### Familial Male Precocious Puberty (Testotoxicosis)

Familial male-limited precocious puberty (FMPP), also known as familial sexual precocity or gonadotropin-independent testotoxicosis, is a disorder in which boys generally develop signs of precocious puberty in early childhood.

The spinal length in boys may be short due to a rapid advance in epiphyseal maturation. FMPP is an autosomal dominant condition with constitutively activating mutations in the luteinizing hormone (LH) receptor, which leads to increased cAMP production, associated with Leydig cell hyperplasia and low sperm cell count (Latronico, A. C. et al., J Clin. Endocrinol. Metab. 80: 2490-2494, 1995; Kosugi, S. et al., Hum. Mol. Genet. 4: 183-188, 1995). PDE4 long form activators described herein are therefore expected to be effective in the treatment, prevention or partial control of FMPP.

### Pituitary Adenomas and Cushing's Disease

Non-cancerous tumours of the pituitary gland are collectively referred to as pituitary adenomas and can lead to hypersecretion of adenohypophyseal hormones (e.g. growth hormone, thyroid stimulating hormone, luteinizing hormone, follicle stimulating hormone and adrenocorticotrophic hormone), which exert their action through GPCRs coupled to Gs and cAMP generation. Thus pituitary adenomas can lead to a state of enhanced cAMP mediated signalling in a variety of endocrine tissues which can precipitate a number of hormonal disorders such as acromegly (mainly due to excess growth hormone secretion), Cushing's disease (due to overproduction of adrenocorticotrophic hormone (ACTH) and the subsequent hypercortisolemia) and/or general hyperpituitarism (associated with excess release of multiple anterior pituitary hormones). Current treatment options for pituitary adenomas include treatment with dopamine receptor agonists, which reduce tumour size and lower pituitary hormonal output through a mechanism involving lowering of intracellular cAMP levels. PDE4 long form activators described herein may also be expected to attenuate the pathological effects of pituitary hormones in their target tissues, such as the adrenal glands.

In Cushing's disease, pituitary adenoma related overproduction of ACTH can lead to hypercortisolemia through an overactivation of melanocortin 2 receptor (MC2) and subsequent cAMP mediated stimulation of steroidogenesis and release of cortisol from the adrenal cortex (Tritos, N. A. and Biller, B. M. Discov. Med. 13: 171-179, 2012). PDE4 long form activators described herein are therefore expected to be effective in the treatment, prevention or partial control of Cushing's disease.

### Polycystic Kidney Disease

Polycystic kidney disease (PKD) is a genetic disorder of the kidneys characterised by development of pathological cysts, which damage renal structure and compromise kidney function (Takiar, V. and Caplan, M. J. Biochim. Biophys. Acta. 1812: 1337-1343, 2011; Masoumi, A. et al., Drugs 67: 2495-2510, 2007). There are two types of PKD: autosomal dominant polycystic kidney disease (ADPKD) and autosomal recessive polycystic kidney disease (ARPKD). ADPKD affects between 0.1% and 0.2% of the population worldwide and is characterized by progressive cyst development and enlarged kidneys. Approximately 50% of people with this disease will develop end stage kidney disease, usually between 40 and 70 years of age and require dialysis or kidney transplantation. ARPKD affects around 1:20,000 live births and is typically identified in the first few weeks after birth. Pulmonary hypoplasia results in a 30-50% death rate in neonates with ARPKD.

Defects in two genes are thought to be responsible for ADPKD. In around 85% of patients, development of ADPKD can be linked to mutations in the gene PKD1, encoding polycystin-1 (PC-1); in around 15% of patients mutations in PKD2, encoding polycystin-2 (PC-2) are implicated. Cyclic AMP has been identified as an important stimulus for proliferation and cyst expansion in polycystic kidney cells but not in normal human kidney cells (Yamaguchi, T. et al., Kidney Int. 57: 1460-1471, 2000). A considerable body of evidence has now developed to implicate cAMP as an important facilitator of renal cystogenesis (Masoumi, A. et al., Drugs 67: 2495-2510, 2007; Wallace, D. P. Biochim. Biophys. Acta. 1812: 1291-1300, 2011). Consistent with the role of cAMP in cyst formation, agents that lower cAMP levels (e.g. vasopressin V2 receptor antagonists and the somatostatin receptor agonist octreotide) showed efficacy in rodent models of PKD (Torres, V. E. et al., Nat. Med. 10: 363-364, 2004; Gattone, V. H. 2^{nd} et al., Nat. Med. 9:1323-1326, 2003; Belibi, F. A. and Edelstein, C. L. Expert Opin. Investig. Drugs. 19: 315-328, 2010). In zebrafish embryos, depletion of a cAMP-hydrolysing PDE enzyme subtype, PDE1A, resulted in development of a cystic phenotype, while PDE1A over-expression partially rescued cystic phenotypes resulting from PC2 depletion (Sussman, C. R., Ward, C. J., Leightner, A. C., Smith, J. L., Agarwal, R., Harris, P. C., Torres, V. E. J. Am. Soc. Nephrol. 25: 2222-2230, 2014). Phosphodiesterase activation has been suggested as a therapeutic strategy for PKD treatment (Sun, Y., Zhou, H. and Yang, B-X. Acta Pharmacologica Sinica 32:805-816, 2011).

PDE4 long form activators described herein are therefore expected to be effective in the treatment, prevention or partial control of polycystic kidney disease.

### Polycystic Liver Disease

Polycystic liver disease (PLD) is a rare inherited condition associated with hepatic cystogenesis (usually defined when number of cysts exceeds 20), which often occurs in association with ADPKD (Strazzabosco, M. and Somlo, S. Gastroenterology 140: 1855-1859, 2011; Gevers, T. J. and Drenth, J. P. Curr. Opin. Gastroenterol. 27: 294-300, 2010). PLD may have a different genetic pathology when compared to ADPKD, driven by mutated proteins associated with the endoplasmic reticulum and the cilium. Increased cholangiocyte proliferation, neovascularisation and elevated fluid secretion act to drive liver cyst formation through dysregulation of multiple signal transduction pathways, including cAMP-mediated signalling. Elevation of hepatic cAMP levels stimulates cAMP-dependent chloride and fluid secretion in biliary epithelial cells and increases cholangiocyte proliferation (Janssen, M. J. et al., J. Hepatol. 52: 432-440, 2010). Somatostatin, which acts through a Gi-coupled mechanism to lower cAMP levels, reduced cholangiocyte proliferation and fluid secretion (Gong, A. Y. et al., Am. J. Physiol. Cell. Physiol. 284: C1205-1214, 2003). Furthermore, the synthetic somatostatin analogue, octreotide, showed efficacy in an animal model of PLD through a mechanism involving reduction in cAMP signalling (Masyuk, T. V. et al., Gastroenterology 132: 1104-1116, 2007). PDE4 long form activators described herein may therefore be effective in the treatment, prevention or partial control of polycystic liver disease due at least in part to cAMP.

### Maturity Onset Diabetes of Young Type 5 (MODY5)

MODY5 is a form of non-insulin-dependent diabetes mellitus associated with renal cysts. It is an autosomal dominant disorder caused by mutations in the gene encoding hepatocyte nuclear factor-1β (HNF-1β). The predominant clinical feature of patients affected by MODY5 is renal dysfunction, frequently diagnosed before the onset of diabetes. In some patients, HNF-1β mutations can result in additional phenotypic features, such as pancreatic atrophy, abnormal liver function and genital tract abnormalities. Studies in mice suggest that the mechanism responsible for renal cyst formation, associated with mutations of HNF-1β, involves a severe defect of the transcriptional activation of PKD2, in addition to effects on uromodulin (UMOD) and PKD1 genes. Down-regulation of PKD1 and PKD2 is associated with cAMP-driven formation of renal cysts (Mancusi, S. et al., J. Nephrol. 26: 207-12, 2013). HNF-1β also binds to the PDE4C promoter and regulates the expression of PDE4C (Ma et al., PNAS 104: 20386, 2007).

PDE4 long form activators described herein are therefore expected to be effective in the treatment, prevention or partial control of the symptoms of MODY5.

### Cardiac Hypertrophy, Heart Failure and Arrhythmia

Localized regulation and integration of cAMP signalling are important for proper cardiac function and perturbation of this signalling can lead to heart failure. Upon chronic β-adrenergic receptor stimulation, cardiomyocyte hypertrophy is induced via elevated cAMP and activation of its downstream effectors, including PKA and Epac (Wang, L. et al., Cell. Signal. 27: 908-922, 2015 and references therein). Cardiomyocyte hypertrophy increases the risk of heart failure and arrhythmia.

PDE4 long form activators described herein may therefore be effective in the treatment, prevention or partial control of cardiac hypertrophy, heart failure and/or arrhythmia.

Diseases Associated with Increased cAMP-Mediated Signalling
Disorders Associated with Activating Mutations of the Alpha Subunit of the G Protein (GNAS1)

The G-protein Gs acts as a transducer for GPCRs that stimulate adenylyl cyclase activity and exert their biological effects by increasing intracellular cAMP levels. Gs is a heterotrimeric protein composed of α, β and γ subunits. Activating mutations in the gene, GNAS1, for the α-subunit have been identified which lead to exaggerated abnormal cAMP signalling in a variety of tissues and give rise to a range of disorders.

### McCune-Albright Syndrome

McCune-Albright syndrome (MAS) is a rare genetic disorder typically characterised by three dominating features of precocious puberty, fibrous dysplasia of bone and café au lait lesions. The underlying molecular pathology for MAS involves an activating mutation of the GNAS1 gene (Diaz, A. Danon, M. and Crawford, J. J. Pediatr. Endocrinol. Metab. 20: 853-880, 2007). PDE4 long form activators described herein would therefore be expected to be effective in the treatment, prevention or partial control of disorders associated with activating mutations of GNAS1, including McCune-Albright syndrome.

### Amelioration of Toxin-Induced Increases in Adenylyl Cyclase Activity in Infectious Diseases.

Adenylyl cyclase, the enzyme responsible for production of cAMP, is a key biological target thought to be involved in mediating the effects of many bacterial toxins (Ahuja et al., Critical Reviews in Microbiology, 30:187-196, 2004). These toxins produce their effects by raising cAMP levels through enhancement of host immune cell and/or pathogen related adenylyl cyclase activity. PDE4 long form activators described herein, by reducing cAMP levels, would therefore be expected to be of utility in the treatment or partial control of symptoms of infectious diseases that are associated with elevated cAMP activity. The following are some examples of such infectious diseases:

### Cholera

Vibrio cholerae produces cholera toxin, which through adenosine disphosphate ribosylation of the a subunit of Gs leads to host cell adenylyl cyclase activation and cAMP production. Diarrhoea caused by cholera toxin is believed to be a result of excessive cAMP accumulation in the cells of the gastrointestinal tract.

### Whooping Cough

Bordetella pertussis is the pathogen responsible for the childhood disease whooping cough. Bordetella pertussis toxin stimulates adenosine disphosphate ribosylation of the a subunit of Gi and indirectly augments cAMP levels in target cells. The bacterium also secretes an invasive adenylyl cyclase, which produces toxic cAMP levels and impairs host immune defence.

### Anthrax

Anthrax is caused by Bacillus anthracis and whilst it is primarily an animal disease it can be transmitted to humans through contact. Anthrax infections are associated with widespread oedema, the development of which is thought to be driven by oedema toxin. The latter is an adenylyl cyclase and is activated by host calmodulin to produce abnormally high levels of cAMP that have a toxic effect on host immune cells.

### Tuberculosis

Mycobacterium tuberculosis expresses a large and diverse range of adenylyl cyclases, which may play a role in virulence and generation of disease pathology. One adenylyl cyclase subtype, RV0386, has been demonstrated to enter host macrophages and elevate intracellular cAMP to cause toxicity (Agarwal et al., Nature, 460: 98-102, 2009).

PDE4 long form activators described herein may therefore be effective in the treatment, prevention or partial control of infectious diseases such as cholera, whooping cough, anthrax and tuberculosis.

Diseases Dependent Upon Activation of PKA by Elevated cAMP.

In eukaryotes, cAMP activates protein kinase A (PKA), which is also known as cAMP-dependent protein kinase. PKA is normally inactive as a tetrameric holoenzyme, consisting of two catalytic and two regulatory units, with the regulatory units blocking the catalytic centres of the catalytic units. cAMP binds to specific locations on the regulatory units of PKA and causes dissociation between the regulatory and catalytic units, thus activating the catalytic units. The active catalytic units catalyse the transfer of phosphate from ATP to specific residues of protein substrates, which may modulate the function of those protein substrates.

PDE4 long form activation reduces cAMP levels and cAMP mediated activation of PKA. PDE4 long form activators described herein would therefore be expected to be of utility in the treatment or partial control of disorders where inhibitors of PKA show evidence of therapeutic effects.

Disorders that are dependent upon activation of PKA by cAMP may be identified by their response to PKA inhibitors such as Rp-8-Br-cAMPS. Rp-8-Br-cAMPS is an analogue of cAMP that occupies the cAMP binding sites of PKA, preventing its dissociation and activation.

### HIV Infection and AIDS

T cells from HIV-infected patients have increased levels of cAMP and are more sensitive to inhibition by Rp-8-Br-cAMPS than are normal T cells. Excessive activation of PKA by cAMP has been associated with the progressive T cell dysfunction in HIV infection (Aandahl, E. M. et al., FASEB J. 12: 855-862, 1998). Furthermore, in vivo administration of Rp-8-Br-cAMPS has been shown to restore T cell responses in retrovirus-infected mice (Nayjib, B. et al., The Open Immunology Journal, 1: 20-24, 2008). PDE4 long form activators described herein are therefore expected to be of utility in the treatment, prevention or partial control of HIV infection and AIDS.

### Common Variable Immunodeficiency (CVID)

In vitro administration of Rp-8-Br-cAMPS has been shown to correct impaired secretion of the cytokine IL-10 by T cells from patients with Common Variable Immunodeficiency (CVID) (Holm, A. M. et al., J. Immunol. 170: 5772-5777, 2003). PDE4 long form activators described herein are therefore expected to be of utility in the treatment, prevention or partial control of CVID.

Diseases Dependent Upon Activation of Either or Both of Epac1 and Epac2 by Elevated cAMP.

In addition to PKA, cAMP activates another intracellular receptor, known as exchange protein directly activated by cAMP (Epac). There are two isoforms of Epac, Epac1 and Epac2, both consisting of a regulatory region that binds cAMP and a catalytic region that promotes the exchange of GDP for GTP on the small G proteins, Rap1 and Rap2 of the Ras family. In addition, Epac proteins exert their functions through interactions with a number of other cellular partners at specific cellular loci. Pathophysiological changes in Epac signalling have been associated with a wide range of diseases (Breckler, M. et al., Cell. Signal. 23: 1257-1266, 2011).

Relevant disorders that are dependent upon activation of Epac proteins by cAMP may be identified by their response to Epac inhibitors, such as ESI-09, a novel non-cyclic nucleotide Epac1 and Epac2 antagonist that is capable of specifically blocking intracellular Epac-mediated Rap1 activation and Akt phosphorylation, as well as Epac-mediated insulin secretion in pancreatic beta cells (Almahariq, M. et al., Mol. Pharmacol. 83: 122-128, 2013).

### Melanoma

Epac1 has been implicated in promoting migration and metastasis in melanoma (Baljinnyam, E. et al., Pigment Cell Melanoma Res. 24: 680-687, 2011 and references cited therein). PDE4 long form activators described herein are therefore expected to be of utility in the treatment, prevention or partial control of melanoma.

### Pancreatic Cancer

It has recently been shown that Epac1 is markedly elevated in human pancreatic cancer cells as compared with normal pancreas or surrounding tissue (Lorenz, R. et al., Pancreas 37: 102-103, 2008).

Pancreatic cancer is often resistant to treatments that are usually effective for other types of cancer. Using the Epac inhibitor ESI-09, a functional role of Epac1 overexpression in pancreatic cancer cell migration and invasion was demonstrated (Almahariq, M. et al., Mol. Pharmacol. 83: 122-128, 2013). These findings are consistent with results based on RNAi silencing techniques and suggest that inhibition of Epac1 signalling could be an effective therapeutic strategy for pancreatic cancer.

PDE4 long form activators described herein would therefore be expected to be of utility in the treatment, prevention or partial control of pancreatic cancer.

Diseases Dependent Upon Modulation of cAMP-Gated Ion Channels by Elevated cAMP.

In addition to activation of PKA and Epac, another effector pathway for elevated cAMP is the activation of cAMP-gated ion channels. PDE4 long form activators described herein would therefore be expected to be of utility in the treatment of disorders where inhibitors of cAMP-gated ion channels show evidence of therapeutic effects.

Diseases Associated with Increased Activity of cAMP Response Element Binding Protein.

The cAMP response element binding protein (CREB) is an important transcription factor involved in the regulation of a variety of cellular functions such as cell proliferation, differentiation, survival, and apoptosis (Cho et al., Crit Rev Oncog, 16: 37-46, 2011). CREB activity is regulated by kinase dependant phosphorylation through a range of extracellular signals, such as stress, growth factors and neurotransmitters. Phosphorylation leads to dimerisation of CREB, and together with other co-activator partner proteins, enables it to bind to promoter regions of target genes containing the cAMP response element (CRE sites) and initiate transcriptional activity. The cAMP pathway (e.g. through cAMP-dependant protein kinase mediated phosphorylation) is an important positive modulator of CREB mediated biological activity. PDE4 long form activators described herein are therefore expected to be of utility in the treatment, prevention or partial control of disorders associated with elevated CREB activity.

### Leukaemia

Bone marrow cells from acute lymphoid and myeloid leukaemia patients have been reported to overexpress CREB protein and mRNA (Crans-Vargas et al., Blood, 99: 2617-9, 2002; Cho et al., Crit Rev Oncog, 16: 37-46, 2011). Furthermore, the increased CREB level correlates with poor clinical response in subjects with acute myeloid leukaemia (Crans-Vargas et al., Blood, 99: 2617-9, 2002; Shankar et al., Cancer Cell, 7: 351-62, 2005). Upregulation of CREB is associated with stimulation of human leukaemia cell growth whilst downregulation inhibits myeloid cell proliferation and survival. PDE4 long form activators described herein would be expected to reduce CREB activity and function through attenuation of cAMP mediated stimulation of CREB and therefore expected to have utility in the treatment, prevention or partial control of acute lymphoid and myeloid leukaemia.

### Prostate Cancer

Abnormal excessive androgen activity is an important driver in the development of prostate cancer as it stimulates the development of intraepithelial neoplasias (Merkle et al., Cellular Signalling, 23: 507-515, 2011). This is strongly supported by the use of androgen ablation approaches, involving chemical or surgical castration, in the treatment of prostate cancer. Cyclic AMP elevating agents such as forskolin can enhance androgen receptor activity through multiple intracellular mechanisms including androgen receptor activation through phosphorylation and/or interaction with CREB. Epac1 activation has also been implicated in promoting cellular proliferation in prostate cancer (Misra, U. K. and Pizzo, S. V. J. Cell. Biochem. 108: 998-1011, 2009; Misra, U. K. and Pizzo, S. V. J. Cell. Biochem. 113: 1488-1500, 2012). PDE4 long form activators described herein are therefore expected to have utility in the treatment, prevention or partial control of prostate cancer.

Diseases Associated with Reduced Activity of cAMP-Hydrolysing PDE Enzymes

Loss-of-function mutations in gene(s) for cAMP-hydrolysing PDE isoforms other than PDE4, such as PDE8 and PDE11, have been detected in a number of diseases (Vezzosi, D. and Bertherat, J., Eur. J. Endocrinol. 165: 177-188, 2011; Levy, I. et al., Curr. Opin. Pharmacol. 11: 689-697, 2011; Azevedo, M. F. and Stratakis, C. A. Endocr. Pract. 17 Suppl 3: 2-7, 2011). These mutations can lead to abnormally high cAMP levels and/or duration of cAMP action with pathological consequences as detailed below. PDE4 long form activators described herein are therefore expected to be of utility in the treatment, prevention or partial control of these diseases, such as adrenocortical tumours, testicular cancer, PPNAD and Carney Complex.

### Adrenocortical Tumours

Adrenocortical tumours associated with an inactivating point mutation in the gene encoding PDE11A4 have decreased expression of PDE11A4 and increased cAMP levels (Horvath, A. et al., Nat Genet. 38: 794-800, 2006; Horvath, A. et al., Cancer Res. 66: 11571-11575, 2006; Libé, R., et al., Clin. Cancer Res. 14: 4016-4024, 2008).

### Testicular Cancer

Mutations that reduce PDE11A activity and increase cAMP levels have been observed in some forms of testicular cancer (Horvath. A. et al., Cancer Res. 69: 5301-5306, 2009).

### Primary Pigmented Nodular Adrenocortical Diseases (PPNAD)

Mutations in the PDE8B gene have also been identified as a predisposing factor for PPNAD and the mutant protein shows reduced ability to degrade cAMP (Horvath, A., Mericq, V. and Stratakis, C. A. N. Engl. J. Med. 358: 750-752, 2008; Horvath, A. et al., Eur. J. Hum. Genet. 16: 1245-1253, 2008).

### Carney Complex

In Carney Complex (CNC) caused by PRKAR1A mutations, some patients also have defects in PDE11A that may exert a synergistic effect to enhance abnormal activation of the cAMP signal transduction pathway, leading to adrenal and testicular cancer (Libé, R. et al., J. Clin. Endocrinol. Metab. 96: E208-214, 2011).

### Treatment and Posology

By “treatment” herein is meant the treatment by therapy, whether of a human or a non-human animal (e.g., in veterinary applications) typically a non-human mammal, in which some desired therapeutic effect on the condition is achieved; for example, the inhibition of the progress of the disorder, including a reduction in the rate of progress, a halt in the rate of progress, amelioration of the disorder or cure of the condition. Treatment as a prophylactic measure is also included. References herein to prevention or prophylaxis do not indicate or require complete prevention of a condition; its manifestation may instead be reduced or delayed via prophylaxis or prevention according to the present invention.

Compounds or compositions as described herein, when used for preventing or treating a disorder, may be administered in an “effective amount”, which may also be referred to as a “therapeutically effective amount”. By a “therapeutically effective amount” herein is meant an amount of the one or more compounds described herein or a pharmaceutical formulation comprising such one or more compounds, which is effective for producing such a therapeutic effect, commensurate with a reasonable benefit/risk ratio.

It will be appreciated that appropriate dosages of the compounds described herein may vary from patient to patient. Determining the optimal dosage will generally involve the balancing of the level of therapeutic benefit against any risk or deleterious side effects of the treatments of the present invention. The selected dosage level will depend on a variety of factors including the activity of the particular compound, the route of administration, the time of administration, the rate of excretion of the compound, the duration of the treatment, other drugs, compounds or materials used in combination and the age, sex, weight, condition, general health and prior medical history of the patient. The amount of compound(s) and route of administration will ultimately be at the discretion of the physician, although generally the dosage will be to achieve local concentrations at the site of action so as to achieve the desired effect. Administration in vivo can be effected in one dose, continuously or intermittently throughout the course of treatment. Methods of determining the most effective means and dosage of administration are well known to a person skilled in the art and will vary with the formulation used for therapy, the purpose of the therapy, the target cell being treated, and the subject being treated. Single or multiple administrations can be carried out with the dose level and pattern being selected by the treating physician.

In general, a suitable dose of the one or more compounds described herein may be in the range of about 0.001 to 50 mg/kg body weight of the subject per day, preferably in a dosage of 0.01-25 mg per kg body weight per day, e.g., 0.01, 0.05, 0.10, 0.25, 0.50, 1.0, 2.5, 10 or 25 mg/kg per day. Where the compound(s) is a salt, solvate, prodrug or the like, the amount administered may be calculated on the basis of the parent compound and so the actual weight to be used may be increased proportionately.

### Combination Therapies

The compounds described herein may also find application in mimicking or enhancing the effects of drugs known to produce their therapeutic effect through lowering of intracellular cAMP levels.

A number of therapeutically beneficial drugs have a primary mode of action involving lowering intracellular cAMP levels and/or cAMP-mediated activity, as summarised below. Since PDE4 long form activators described herein will also act to lower cAMP levels it is expected that these agents will mimic and/or augment the pharmacological properties and therapeutic utility of drugs operating through a down-regulation of cAMP-mediated signalling. In certain embodiments, a compound described herein is therefore provided as part of a combination therapy with another agent that lowers intracellular cAMP levels and/or cAMP-mediated activity. The combination therapy may be administered simultaneously, contemporaneously, sequentially or separately. In one embodiment, the compound described herein and the separate cAMP lowering agent are provided in a single composition, as described in more detail below. The combination therapy may comprise a described herein and one or more of:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.
   Combination with α-2 Adrenergic Receptor Agonist

α-2 Adrenergic receptor stimulation is known to reduce cAMP levels through a Gᵢ protein-mediated inhibition of adenylyl cyclase activity in a broad range of tissues. In noradrenergic neurones in the brain and peripheral sympathetic nervous system, presynaptic α-2 adrenergic receptor activation inhibits noradrenaline release and noradrenergic activity. Drugs (e.g. clonidine, dexmedetomidine, guanfacine) that act as agonists at these receptors are effective in the treatment of a variety of clinical conditions. Clonidine, the prototypic agent, has shown therapeutic utility in the treatment of hypertension, neuropathic pain, opioid detoxification, insomnia, ADHD, Tourette syndrome, sleep hyperhidrosis, addiction (narcotic, alcohol and nicotine withdrawal symptoms), migraine, hyperarousal, anxiety and also as a veterinary anaesthetic. Lowering of cAMP levels by PDE4 long form activation may be expected to yield similar effects to drugs acting through α-2 adrenergic receptor stimulation. Furthermore, PDE4 long form activators described herein may be expected to potentiate the pharmacodynamic effects of α-2 adrenergic receptor agonists when used in combination.

Combination with β-1 Adrenergic Receptor Antagonist

β-1 Adrenergic receptor antagonists are used in the treatment a range of cardiovascular indications including hypertension, cardiac arrhythmias and cardioprotection following myocardial infarction. Their primary mechanism of action involves reducing the effects of excessive circulating adrenaline and sympathetic activity, mediated by noradrenaline, particularly at cardiac β-1 adrenergic receptors. Endogenous and synthetic β-1 adrenergic receptor agonists stimulate adenylyl cyclase activity through Gₛ activation and raise intracellular cAMP levels in a variety of tissues such as heart and kidney. Consequently, drugs that block β-1 adrenergic receptor mediated activity exert their pharmacological effects by attenuating the increase in cAMP mediated signalling. Given that PDE4 long form activation will also lower cAMP concentration and transduction in cardiac tissue, PDE4 long form activators described herein may be expected to find utility in the treatment or partial control of hypertension, cardiac arrhythmias, congestive heart failure and cardioprotection. Additional non-cardiovascular therapeutic utility may be expected in disorders such as post-traumatic stress related disorder, anxiety, essential tremor and glaucoma, which also respond to β-1 adrenergic antagonist treatment. Furthermore, PDE4 long form activators described herein may be expected to potentiate the pharmacodynamic effects of β-1 adrenergic receptor antagonists when used in combination.

### Methods of Treatment

Compounds as described herein may be used for treating or preventing a disease or disorder that can be ameliorated by activation of long isoforms of PDE4. Compounds as described herein may be used for treating or preventing a disease or disorder mediated by excessive intracellular cyclic AMP signalling. Compounds as described herein may be used for treating or preventing a disease or disorder that can be ameliorated by activation of long isoforms of PDE4, wherein the disease or disorder that can be ameliorated by activation of long isoforms of PDE4 is a disease or disorder mediated by excessive intracellular cyclic AMP signalling. In a further aspect, the present invention provides a small molecule activator of a PDE4 long form described herein for use in a method for the treatment or prevention of a disease or disorder in a patient in need of such therapy. The invention also provides a method of treating or preventing a disease or disorder in a patient in need thereof, comprising administering to a patient in need thereof an effective amount of a compound described herein. The invention provides a method of treating or preventing a disease or disorder that can be ameliorated by activation of long isoforms of PDE4, comprising administering to a patient in need thereof a therapeutically effective amount of any compound or a pharmaceutically acceptable salt or derivative as described herein. The invention provides a method of treating or preventing a disease or disorder mediated by excessive intracellular cyclic AMP signalling, comprising administering to a patient in need thereof a therapeutically effective amount of any compound or a pharmaceutically acceptable salt or derivative as described herein. The disease or disorder may be any disease of disorder described herein, including: a disease associated with increased cAMP production and signalling (such as hyperthyroidism, Jansens's metaphyseal chondrodysplasia, hyperparathyroidism, familial male-limited precocious puberty, pituitary adenomas, Cushing's disease, polycystic kidney disease, polycystic liver disease, MODY5 and cardiac hypertrophy); diseases known to be associated with increased cAMP-mediated signalling, including disorders associated with activating mutations of the alpha subunit of the G protein (GNAS1) (such as McCune-Albright syndrome); amelioration of toxin-induced increases in adenylyl cyclase activity in infectious diseases (such as cholera, whooping cough, anthrax, and tuberculosis); treatment of diseases known to be dependent upon activation of PKA by elevated cAMP (such as HIV infection and AIDS, and Common Variable Immunodeficiency (CVID)); treatment of diseases known to be dependent upon activation of either or both of Epac1 and Epac2 by elevated cAMP (such as melanoma and pancreatic cancer); treatment of diseases dependent upon modulation of cAMP-gated ion channels by elevated cAMP; treatment of diseases known to be associated with increased activity of cAMP response element binding protein (such as leukaemia and prostate cancer); treatment of diseases known to be associated with reduced activity of cAMP-hydrolysing PDE enzymes (such as adrenocortical tumours, testicular cancer, primary pigmented nodular adrenocortical diseases (PPNAD) and Carney Complex); and mimicking or enhancing the effects of drugs known to produce their therapeutic effect through lowering of intracellular cAMP levels.

As used herein, the terms “compound of the invention”, “compound of the disclosure” “compound described herein” and “compound of Formula I”, etc, include pharmaceutically acceptable salts and derivatives thereof and polymorphs, isomers (e.g. stereoisomers and tautomers) and isotopically labelled variants thereof. For example, reference to compounds of Formula I includes pharmaceutically acceptable salts thereof. Reference to compounds of Formula Ia includes pharmaceutically acceptable salts thereof. Furthermore, these terms include all the sub-embodiments of those compounds disclosed herein, including compounds of Formula I to V and Ia to Va, and all embodiments thereof.

A compound described herein may be provided as a solvate, for example a hydrate.

Pharmaceutically acceptable derivatives of a compound described herein include pharmaceutically acceptable esters, amides, prodrugs (e.g. a pyridine N-oxide) or isotopically labelled variants thereof. The present invention further provides pharmaceutical compositions comprising a compound described herein, including a pharmaceutically acceptable salt, solvate, ester, hydrate or amide thereof, in admixture with a pharmaceutically acceptable excipient(s), and optionally other therapeutic agents. The term “acceptable” means being compatible with the other ingredients of the composition and not deleterious to the recipient thereof. Compositions include e.g. those suitable for oral, sublingual, subcutaneous, intravenous, epidural, intrathecal, intramuscular, transdermal, intranasal, pulmonary, topical, local, or rectal administration, and the like, typically in unit dosage forms for administration.

The term “pharmaceutically acceptable salt” includes a salt prepared from pharmaceutically acceptable non-toxic acids or bases including inorganic or organic acids and bases. Compounds which contain basic, e.g. amino, groups are capable of forming pharmaceutically acceptable salts with acids. Examples of pharmaceutically acceptable acid addition salts of the compounds described herein include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Compounds which contain acidic, e.g. carboxyl, groups are capable of forming pharmaceutically acceptable salts with bases. Pharmaceutically acceptable basic salts of the compounds described herein include, but are not limited to, metal salts such as alkali metal or alkaline earth metal salts (e.g. sodium, potassium, magnesium or calcium salts) and zinc or aluminium salts and salts formed with ammonia or pharmaceutically acceptable organic amines or heterocyclic bases such as ethanolamines (e.g. diethanolamine), benzylamines, N-methyl-glucamine, amino acids (e.g. lysine) or pyridine.

Hemisalts of acids and bases may also be formed, e.g. hemisulphate salts.

Pharmaceutically acceptable salts of compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

### Prodrugs

Compounds described herein may be provided as a prodrug. Prodrugs are derivatives of compounds described herein (which may have little or no pharmacological activity themselves), which can, when administered in vivo, be converted into compounds described herein.

Prodrugs can, for example, be produced by replacing functionalities present in the compounds described herein with appropriate moieties which are metabolised in vivo to form a compound described herein. The design of prodrugs is well-known in the art, as discussed in Bundgaard, Design of Prodrugs 1985 (Elsevier), The Practice of Medicinal Chemistry 2003, 2^{nd} Ed, 561-585 and Leinweber, Drug Metab. Res. 1987, 18: 379.

In vivo metabolism of prodrugs of compounds described herein may for example involve hydrolysis, oxidative metabolism or reductive metabolism of the prodrug. Examples of prodrugs of compounds described herein are amides and esters of those compounds that may be hydrolysed in vivo. For example, where the compound described herein contains a carboxylic acid group (—COOH), the hydrogen atom of the carboxylic acid group may be replaced in order to form an ester (e.g. the hydrogen atom may be replaced by C₁₋₆alkyl). Where a compound contains an alcohol group (—OH), the hydrogen atom of the alcohol group may be replaced in order to form an ester (e.g. the hydrogen atom may be replaced by —C(O)C₁₋₆alkyl). Further examples of prodrugs of compounds described herein include pyridine N-oxides that may be reductively metabolised in vivo to form compounds described herein containing a pyridine ring.

### Solvates

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

### Isomers

It will be appreciated that the compounds described herein may exist in various isomeric forms and the compounds described herein include all stereoisomeric forms and mixtures thereof, including enantiomers and racemic mixtures. The present invention includes within its scope the use of any such stereoisomeric form or mixture of stereoisomers, including the individual enantiomers of the compounds described herein as well as wholly or partially racemic mixtures of such enantiomers. Where appropriate, isomers can be separated from their mixtures by the application or adaptation of known methods (e.g. chromatographic techniques and recrystallisation techniques). Where appropriate, isomers can be prepared by the application or adaptation of known methods (e.g. asymmetric synthesis). In addition, it will be appreciated that in some instances, compounds described herein may exist in tautomeric forms and the compounds described herein include all tautomers and mixtures thereof.

### Isotopes

The compounds described herein include pharmaceutically acceptable isotopically-labelled compounds wherein one or more atoms are replaced by atoms having the same atomic number, but an atomic mass or mass number different from the atomic mass or mass number usually found in nature.

Examples of isotopes suitable for inclusion in the compounds described herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulphur, such as ³⁵S. Certain isotopically-labelled compounds, for example, those incorporating a radioactive isotope, are useful in drug and/or substrate tissue distribution studies. The radioactive isotopes ³H and ¹⁴C are particularly useful for this purpose in view of their ease of incorporation and ready means of detection. Substitution with positron emitting isotopes, such as ¹¹C, ¹⁸F, ¹⁵O and ¹³N, can be useful in Positron Emission Topography (PET) studies for examining substrate receptor occupancy. It is well known in the art that isotope substitution of a hydrogen atom that is bonded to carbon with deuterium [²H] may positively influence the ADME properties of drug candidates by slowing CYP-mediated metabolism [for a review see Nat. Rev. Drug Discov. 15(4): 219-21 (2016)].

Isotopically-labelled compounds can generally be prepared by conventional techniques known to those skilled in the art or by processes analogous to those described herein using an appropriate isotopically-labelled reagent in place of the non-labelled reagent previously employed.

### Pharmaceutical Compositions

A pharmaceutical composition may comprise any compound or a pharmaceutically acceptable salt or derivative as described herein, and a pharmaceutically acceptable excipient. A pharmaceutical composition as described herein may comprise one or more pharmaceutically acceptable excipients, for example pharmaceutically acceptable carriers, diluents, preserving agents, solubilising agents, stabilising agents, disintegrating agents, binding agents, lubricating agents, wetting agents, emulsifiers, sweeteners, colourants, odourants, salts, buffers, coating agents and antioxidants. Suitable excipients and techniques for formulating pharmaceutical compositions are well known in the art (see, e.g. Remington: The Science and Practice of Pharmacy, 20th Ed., ed. A. Gennaro, Lippincott Williams & Wilkins, 2000). Suitable excipients include, without limitation, pharmaceutical grade starch, mannitol, lactose, corn starch, magnesium stearate, stearic acid, alginic acid, sodium saccharin, talcum, cellulose, cellulose derivatives (e.g. hydroxypropylmethylcellulose, carboxymethylcellulose) glucose, sucrose (or other sugar), sodium carbonate, calcium carbonate, magnesium carbonate, sodium phosphate, calcium phosphate, gelatin, agar, pectin, liquid paraffin oil, olive oil, alcohol, detergents, emulsifiers or water (preferably sterile).

A pharmaceutical composition may further comprise an adjuvant and/or one or more additional therapeutically active agent(s).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by oral, buccal or sublingual routes or parenteral routes, including subcutaneous, intramuscular, intravenous, intraperitoneal, and intradermal, rectal and topical administration, and inhalation. Such compositions may be prepared by any method known in the art of pharmacy, for example by admixing the active ingredient with a excipient(s) under sterile conditions.

For oral administration, the active ingredient may be presented as discrete units, such as tablets, capsules, powders, granulates, solutions, suspensions, and the like.

Formulations suitable for oral administration may also be designed to deliver the compounds described herein in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds described herein may also be used in fast-dissolving, fast-disintegrating dosage forms such as those described in Liang and Chen, Expert Opinion in Therapeutic Patents 2001, 11(6): 981-986.

The formulation of tablets is discussed in H. Lieberman and L. Lachman, Pharmaceutical Dosage Forms: Tablets 1980, vol. 1 (Marcel Dekker, New York).

For administration intranasally or by inhalation, the active ingredient may be presented in the form of a dry powder from a dry powder inhaler or in the form of an aerosol spray of a solution or suspension from a pressurised container, pump, spray, atomiser or nebuliser.

For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

For parenteral administration, the compounds described herein may be administered directly into the blood stream, into subcutaneous tissue, into muscle, or into an internal organ. Suitable means for administration include intravenous, intraarterial, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions. Where the solution is aqueous, excipients such as sugars (including but not restricted to glucose, mannitol, sorbitol, etc.) salts, carbohydrates and buffering agents (preferably to a pH of from 3 to 9) may be used. For some applications, the compounds described herein may be more suitably formulated as a sterile non-aqueous solution or as a dried form to be used in conjunction with a suitable vehicle such as sterile, pyrogen-free water (WFI).

Parenteral formulations may include implants derived from degradable polymers such as polyesters (e.g. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds described herein used in the preparation of parenteral solutions may be increased by the use of appropriate formulation techniques, such as the incorporation of co-solvents and/or solubility-enhancing agents such as surfactants, micelle structures and cyclodextrins.

Mixed with such pharmaceutically acceptable excipients, e.g. as described in the standard reference, Gennaro, A. R. et al, Remington: The Science and Practice of Pharmacy (21st Edition, Lippincott Williams & Wilkins, 2005, see especially Part 5: Pharmaceutical Manufacturing), the active agent may be compressed into solid dosage units, such as pills, tablets, or be processed into capsules, suppositories or patches. By means of pharmaceutically acceptable liquids the active agent can be applied as a fluid composition, e.g. as an injection preparation or as an aerosol spray, in the form of a solution, suspension, or emulsion.

For making solid dosage units, the use of conventional additives such as fillers, colorants, polymeric binders and the like is contemplated. In general any pharmaceutically acceptable additive that does not interfere with the function of the active compounds can be used. Suitable carriers with which the active agent described herein can be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The invention further includes a pharmaceutical composition, as hereinbefore described, in combination with packaging material suitable for said composition, said packaging material including instructions for the use of the composition for the use as hereinbefore described. In some embodiments, the one or more compounds described herein may be used in combination therapies for the treatment of the described conditions i.e., in conjunction with other therapeutic agents. For the case of active compounds combined with other therapies the two or more treatments may be given in individually varying dose schedules and via different routes.

The combination of the agents listed above with a compound described herein would be at the discretion of the physician who would select dosages using his common general knowledge and dosing regimens known to a skilled practitioner.

Where a compound described herein is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

In one embodiment, the invention provides a product comprising a compound described herein and another therapeutic agent as a combined preparation for simultaneous, separate or sequential use in therapy. In one embodiment, the therapy is the treatment or prevention of disorders where a reduction of second messenger responses mediated by cyclic 3′,5′-adenosine monophosphate (cAMP) is required. Products provided as a combined preparation include a composition comprising a compound described herein and the other therapeutic agent together in the same pharmaceutical composition, or the compound described herein and the other therapeutic agent in separate form, e.g. in the form of a kit.

In one embodiment, the invention provides a pharmaceutical composition comprising a compound of the invention and another therapeutic agent. Optionally, the pharmaceutical composition may comprise a pharmaceutically acceptable excipient, as described above.

In one embodiment, the invention provides a kit comprising two or more separate pharmaceutical compositions, at least one of which contains a compound described herein. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound described herein and the other therapeutic agent may be manufactured and/or formulated by the same or different manufacturers. Moreover, the compound described herein and the other therapeutic may be brought together into a combination therapy: (i) prior to release of the combination product to physicians (e.g. in the case of a kit comprising the compound described herein and the other therapeutic agent); (ii) by the physician themselves (or under the guidance of the physician) shortly before administration; (iii) in the patient themselves, e.g. during sequential administration of the compound described herein and the other therapeutic agent.

### Method of Manufacture & Method of Treatment

The invention also provides the use of a compound described herein in the manufacture of a medicament for the treatment or prevention of disorders where a reduction of second messenger responses mediated by cyclic 3′,5′-adenosine monophosphate (cAMP) is required, wherein the medicament is prepared for administration with another therapeutic agent. The invention also provides the use of another therapeutic agent in the manufacture of medicament for treating a disease or condition mediated by cAMP for the treatment or prevention of disorders where a reduction of second messenger responses mediated by cAMP is required, wherein the medicament is prepared for administration with a compound described herein.

The invention also provides a compound described herein for use in the treatment or prevention of disorders where a reduction of second messenger responses mediated by cAMP is required, wherein the compound described herein is prepared for administration with another therapeutic agent. The invention also provides another therapeutic agent for use in the treatment or prevention of disorders where a reduction of second messenger responses mediated by cAMP is required, wherein the other therapeutic agent is prepared for administration with a compound described herein. The invention also provides a compound described herein for use in for the treatment or prevention of disorders where a reduction of second messenger responses mediated by cAMP is required, wherein the compound described herein is administered with another therapeutic agent. The invention also provides another therapeutic agent for use in the treatment or prevention of disorders where a reduction of second messenger responses mediated by cAMP is required, wherein the other therapeutic agent is administered with a compound described herein.

The invention also provides the use of a compound described herein in the manufacture of a medicament for the treatment or prevention of disorders where a reduction of second messenger responses mediated by cAMP is required, wherein the patient has previously (e.g. within 24 hours) been treated with another therapeutic agent. The invention also provides the use of another therapeutic agent in the manufacture of a medicament for the treatment or prevention of disorders where a reduction of second messenger responses mediated by cAMP is required, wherein the patient has previously (e.g. within 24 hours) been treated with a compound described herein.

In one embodiment, the other therapeutic agent is:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.

### EXAMPLES

The present invention will now be further described by way of the following non-limiting examples and with reference to the Tables:

Table 1 shows the structures of small molecule PDE4 long form activators according to the present invention.

Table 2 shows enzyme assay data for PDE4D5, a long form of PDE4 and PDE4B2, a short form of PDE4.

Table 3 shows a reduction of cAMP levels in a 3D culture of m-IMCD3 kidney cells treated with compounds of the present invention.

Table 4 shows inhibition of PGE2-stimulated cyst formation in a 3D culture of m-IMCD3 kidney cells treated with compounds of the present invention.

### General Experimental Details

Reactions were monitored by thin layer chromatography (Merck Millipore TLC Silica Gel 60 F₂₅₄). Flash column chromatography was performed on Biotage Isolera® or Büchi Reveleris® X2 Flash Chromatography systems using pre-packed silica gel columns. NMR spectra were recorded using Bruker 300 or 400 MHz spectrometers, using residual signal of deuterated solvent as internal reference at 25° C. (unless otherwise specified). Exchangeable NH and OH residues were not identifiable in the ¹H-NMR spectra in some cases.

### UPLC Methods:

Method A: Instrument: Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5 μm) Flow: 0.8 mL/min Column temp: 40° C., Eluent A: 0.1% Formic acid in Water, Eluent B: 0.1% Formic acid in Acetonitrile, Gradient: t=0 min 5% B, t=0.5 min 5% B, t=4.5 min 98% B; t=5 min 98% B, Postrun: 0.5 min.

Method B: Instrument: Agilent 1290 Infinity II, 1290 G7120A Bin. Pump, 1290 G7167B Multisampler, 1290 MCT G7116B Column Comp., 1290 G7117B DAD (210-320 nm), PDA (210-320 nm), G6135B MSD (ESI pos/neg) mass range: 90-1500, Column: XSelect CSH XP C18 (50×2.1 mm, 2.5 μm) Flow: 0.8 mL/min Column temp: 25, Eluent A: 10 mM Ammonium Bicarbonate in Water (pH 9.5), Eluent B: Acetonitrile, Gradient: t=0 min 5% B, t=0.5 min 5% B, t=4.5 min 98% B; t=5 min 98% B, Postrun: 0.5 min.

Method C: Instrument: Agilent 1290 Infinity II, 1290 G7120A Bin. Pump, 1290 G7167B Multisampler, 1290 MCT G7116B Column Comp., 1290 G7117B DAD (210, 210-320 nm), PDA (210-320 nm), G6135B MSD (ESI pos/neg) mass range: 90-1500, Column: Atlantis T3 (100×3.0 mm, 3 μm) Flow: 0.8 mL/min Column temp: 40° C., Eluent A: 0.1% Formic acid in Water, Eluent B: 0.1% Formic acid in Acetonitrile, Gradient: t=0 min 5% B, t=1 min 5% B, t=10 min 98% B; t=12 min 98% B, Postrun: 2.5 min.

Method D: Instrument: Agilent 1290 Infinity II, 1290 G7120A Bin. Pump, 1290 G7167B Multisampler, 1290 MCT G7116B Column Comp., 1290 G7117B DAD (210, 210-320 nm), PDA (210-320 nm), G6135B MSD (ESI pos/neg) mass range: 90-1500, Column: Atlantis T3 (100×2.1 mm, 1.7 μm) Flow: 0.8 mL/min Column temp: 40° C., Eluent A: 0.1% Formic acid in Water, Eluent B: 0.1% Formic acid in Acetonitrile, Gradient: t=0 min 2% B, t=2 min 2% B, t=10 min 30% B; t=12 min 30% B, Postrun: 2.5 min.

### LCMS Methods

Method A: Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6135B MSD (ESI pos/neg) mass range 90-1500, 1290 G7102A ELSD (Evap: 50° C., Neb: 50° C., gasflow: 1.3 mL/min), Column: XSelect CSH C18 (30×2.1 mm 3.5 μm) Flow: 1 mL/min, Column temp.: 40° C., Eluent A: 0.1% Formic acid in Water, Eluent B: 0.1% Formic acid in Acetonitrile, Gradient: t=0 min 5% B, t=1.6 min 98% B, t=3 min 98% B, Postrun: 1.3 min.

Method B: Instrument: Agilent 1260 Infinity, 1260 G1312B Bin. Pump, 1260 G1367E WPS, 1260 TCC G1316A Column Comp. 1260 G1315C DAD (210-320 nm, 210 and 220 nm), PDA (210-320 nm), G6130B MSD (ESI pos/neg) mass range: 100-1000, Column: Waters XSelect CSH C18 (30×2.1 mm, 3.5 μm), Flow: 1 mL/min Column temp: 25, Eluent A: 10 mM ammonium bicarbonate in water (pH=9), Eluent B: Acetonitrile, Gradient: t=0 min 5% B, t=1.6 min 98% B, t=3 min 98% B, Postrun: 1.4 min.

Method C: Instrument: Agilent 1260 Infinity II, 1260 G7112B Bin. Pump, 1260 G7167A Multisampler, 1290 MCT G7116B Column Comp. 1260 G7115A DAD (210, 220 and 210-320 nm), PDA (210-320 nm), G6135B MSD (ESI pos/neg) mass range 90-1500, 1290 G7102A ELSD (Evap: 50° C., Neb: 50° C., gasflow: 1.3 mL/min), Column: XSelect CSH C18 (50×2.1 mm 3.5 μm) Flow: 0.8 mL/min, Column temp.: 60° C., Eluent A: 0.1% Formic acid in Water, Eluent B: 0.1% Formic acid in Acetonitrile, Gradient: t=0 min 5% B, t=4 min 98% B, t=6 min 98% B, Postrun: 2 min.

### HPLC Methods

Method A: (preparative HPLC with formic acid as buffer): MS instrument type: ACQ-SQD2; HPLC instrument type: Waters Modular Preparative HPLC System; column: Waters XSelect (C18, 100×30 mm, 10 μm); flow: 55 mL/min prep pump; column temp: RT; eluent A: 0.1% formic acid in water; eluent B: 100% acetonitrile; lin. gradient: t=0 min 2% B, t=4 min 2% B, t=13 min 30% B, t=14.5 100% B, t=17 min 100% B; detection: DAD (220-320 nm); detection: MSD (ESI pos/neg) mass range: 100-800; fraction collection based on MS and DAD

Method B: (preparative HPLC with ammonium bicarbonate as buffer): MS instrument type: Agilent Technologies G6130B Quadrupole; HPLC instrument type: Agilent Technologies 1290 preparative LC; Column: Waters XSelect CSH (C18, 150×19 mm, 10 μm); Flow: 25 mL/min; Column temp: RT; Eluent A: 10 mM ammonium bicarbonate in water pH=9.5; Eluent B: 100% acetonitrile; lin. gradient: t=0 min 10% B, t=2.5 min 10% B, t=11 min 50% B, t=13 min 100% B, t=17 min 100% B; Detection: DAD (220-320 nm); Detection: MSD (ESI pos/neg) mass range: 100-1000; Fraction collection based on MS and DAD.

### Abbreviations:

| | Name | CAS nr |
|---|---|---|
| RuPhos | 2-dicyclohexylphosphino-2′,6′- | 787618-22-8 |
| | diisopropoxybiphenyl | |
| Pd(Ph₃P)₄ | tetrakis(triphenylphosphine)palladium(0) | 14221-01-3 |
| Xantphos | 9,9-dimethyl-4,5-bis(diphenylphosphino)xanthene | 161265-03-8 |
| Pd₂(dba)₃ | tris(dibenzylideneacetone)dipalladium(0) | 51364-51-3 |
| XPhos | 2-dicyclohexylphosphino-2′,4′,6′- | 564483-18-7 |
| | triisopropylbiphenyl | |
| (^{t}Bu)PhCPhos | 2-[(tert-butyl)phenylphosphino]-2′,6′- | 1660153-91-2 |
| | bis(N,N)dimethylamino)biphenyl | |
| PdCl₂(dppf)—CH₂Cl₂ | [1,1′-bis(diphenylphosphino)ferrocene] | 95464-05-4 |
| | dichloropalladium(II), complex with dichloromethane | |

The following abbreviations may also be used in the experimental details: CDI (1,1′-carbonyldiimidazole), DCM (dichloromethane), DIPEA (N,N-diisopropylethylamine), DMF (N,N-dimethylformamide), EDC (N-ethyl-N′-(3-dimethylaminopropyl) carbodiimide), h (hours), HOBt (hydroxybenzotriazole), MW (microwave), r.t. (room temperature), SEM [2-(trimethylsilyl) ethoxymethyl], TBDPS (tert-butyldiphenylsilyl), THF (tetrahydrofuran). The following abbreviations are used in the assignment of NMR signals: s (singlet), d (doublet), t (triplet), q (quartet), m (multiplet), app. (approximate), br. (broad), dd (double doublet), dt (double triplet), td (triple doublet).

### Synthetic Procedures for Key Intermediates:

### Intermediate 1: Synthesis of benzyl 3,8-diazabicyclo[3.2.1]octane-3-carboxylate

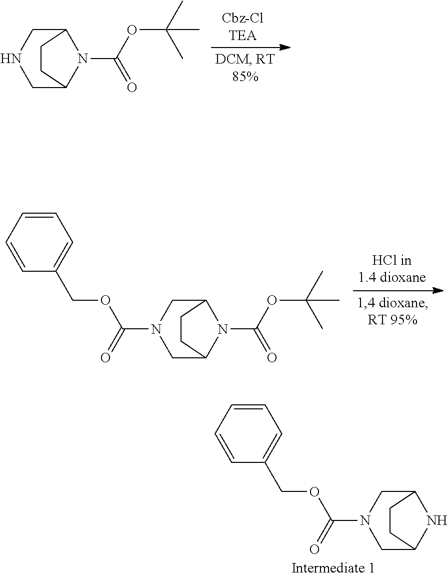 [SMILES: CC(C)(C)OC(=O)N1C2CCC1CNC2 ; CC(C)(C)OC(=O)N1C2CCC1CN(C(=O)OCc1ccccc1)C2 ; O=C(OCc1ccccc1)N1CC2CCC(C1)N2]

Under a nitrogen atmosphere, tert-butyl 3,8-diazabicyclo[3.2.1]octane-8-carboxylate (5.00 g, 23.6 mmol) and triethylamine (6.57 mL, 47.1 mmol) were dissolved in dichloromethane (100 mL) and benzyl chloroformate (3.7 mL, 26 mmol) was slowly added. The mixture was stirred at room temperature for 30 minutes, poured into saturated aqueous sodium bicarbonate solution and the layers were separated. The aqueous layer was extracted with dichloromethane twice. The combined organic layers were washed with brine, dried with sodium sulfate and concentrated in vacuo to afford a yellow oil. The crude oil was purified with silica column chromatography (0% to 50% ethyl acetate in n-heptane) and concentrated in vacuo to afford 3-benzyl 8-(tert-butyl) 3,8-diazabicyclo[3.2.1]octane-3,8-dicarboxylate (8.15 g, 85%) as a colorless oil.

To a solution of 3-benzyl 8-(tert-butyl) 3,8-diazabicyclo[3.2.1]octane-3,8-dicarboxylate (8.15 g, 20.0 mmol) in 1,4-dioxane (40 mL) was added 4 M hydrochloric acid in 1,4-dioxane (40 mL, 160 mmol) and the mixture was stirred at room temperature for 3 hours. The mixture was concentrated in vacuo and partitioned between ethyl acetate and saturated aqueous sodium bicarbonate solution. The layers were separated, and the aqueous layer was extracted with ethyl acetate twice. The combined organic layers were washed with brine, dried with sodium sulfate and concentrated in vacuo to afford benzyl 3,8-diazabicyclo[3.2.1]octane-3-carboxylate (4.95 g, 95%, Intermediate 1) as a light-yellow oil. ¹H-NMR: δ_{H} (400 MHz, CDCl₃) 7.39-7.28 (5H, m), 5.13 (2H, s), 3.89-3.72 (2H, m), 3.57-3.41 (2H, m), 3.13-2.97 (2H, m), 1.86 (1H, s), 1.80-1.66 (4H, m).

### Intermediate 2: Synthesis of benzyl 8-(4,5,6,7-tetrahydrothiazolo[5,4-c]pyridin-2-yl)-3,8-diazabicyclo[3.2.1]octane-3-carboxylate

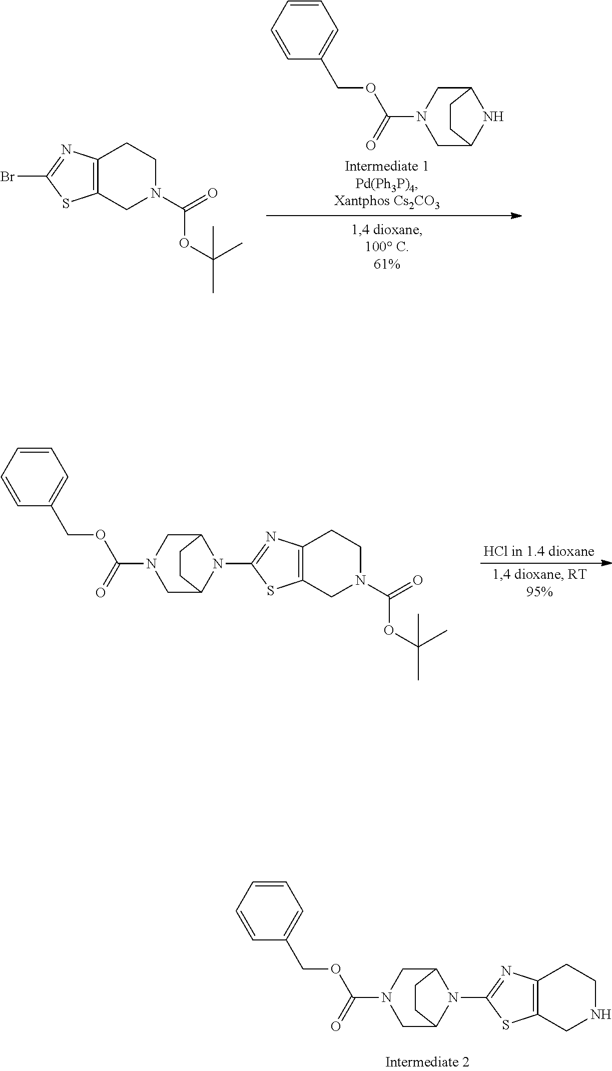 [SMILES: CC(C)(C)OC(=O)N1CCc2nc(Br)sc2C1 ; O=C(OCc1ccccc1)N1CC2CCC(C1)N2 ; CC(C)(C)OC(=O)N1CCc2nc(N3C4CCC3CN(C(=O)OCc3ccccc3)C4)sc2C1 ; O=C(OCc1ccccc1)N1CC2CCC(C1)N2c1nc2c(s1)CNCC2]

Under an argon atmosphere, tert-butyl 2-bromo-6,7-dihydrothiazolo[5,4-c]pyridine-5(4H)-carboxylate (500 mg, 1.57 mmol, 4), benzyl 3,8-diazabicyclo[3.2.1]octane-3-carboxylate (424 mg, 1.72 mmol, Intermediate 1) and cesium carbonate (1.53 g, 4.70 mmol) were dissolved in 1,4-dioxane (20 mL). Argon gas was bubbled through for 5 minutes. Next, Pd(Ph₃P)₄ (91 mg, 0.08 mmol) and Xantphos (91 mg, 0.16 mmol) were added, and the mixture was stirred at 100° C. for 20 hours. The mixture was cooled to room temperature, poured into water and extracted with ethyl acetate twice. The combined organic layers were washed with brine, dried with sodium sulfate and concentrated in vacuo to afford a yellow oil. The oil was purified with silica column chromatography (0% to 100% ethyl acetate in n-heptane) and concentrated in vacuo to afford tert-butyl 2-(3-((benzyloxy)carbonyl)-3,8-diazabicyclo[3.2.1]octan-8-yl)-6,7-dihydrothiazolo[5,4-c]pyridine-5(4H)-carboxylate (503 mg, 61%) as a yellow gum. To a solution of tert-butyl 2-(3-((benzyloxy)carbonyl)-3,8-diazabicyclo[3.2.1]octan-8-yl)-6,7-dihydrothiazolo[5,4-c]pyridine-5(4H)-carboxylate (500 mg, 1.03 mmol) in 1,4-dioxane (10 mL) was added 4 M hydrochloric acid in 1,4-dioxane (5 mL, 20.0 mmol) and the mixture was stirred at room temperature for 16 hours. The mixture was concentrated in vacuo, redissolved in methanol and purified with SCX (ion exchange) chromatography (washed with methanol and eluted with 3.5 M ammonia in methanol) to afford benzyl 8-(4,5,6,7-tetrahydrothiazolo[5,4-c]pyridin-2-yl)-3,8-diazabicyclo[3.2.1]octane-3-carboxylate (360 mg, 86%, Intermediate 2) as a light-yellow gum. ¹H-NMR: δ_{H} (400 MHz, CDCl₃) 7.40-7.29 (5H, m), 5.14 (2H, s), 4.27-4.15 (2H, m), 3.92-3.86 (3H, m), 3.79 (1H, d, J 12.8), 3.45-3.29 (2H, m), 3.24-3.14 (3H, m), 2.69-2.60 (2H, m), 2.08-1.99 (2H, m), 1.80-1.72 (2H, m).

### Intermediate 3: Synthesis of benzyl 8-(5,6-dihydro-4H-pyrrolo[3,4-d]thiazol-2-yl)-3,8-diazabicyclo[3.2.1]octane-3-carboxylate

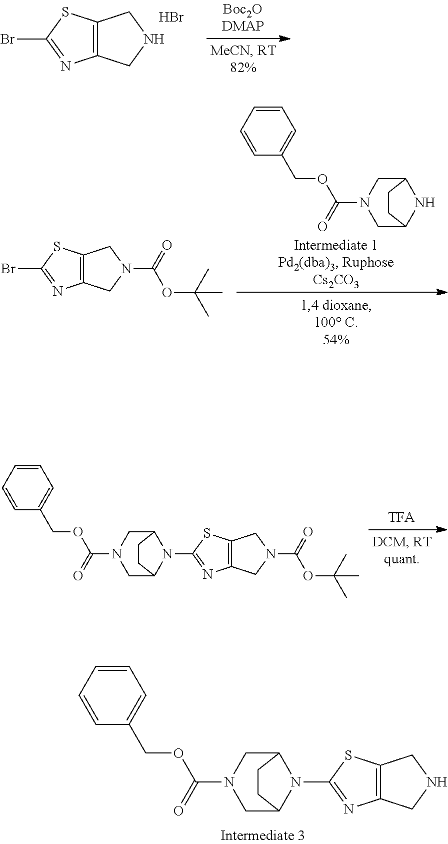 [SMILES: Brc1nc2c(s1)CNC2 ; Br ; O=C(OCc1ccccc1)N1CC2CCC(C1)N2 ; CC(C)(C)OC(=O)N1Cc2nc(Br)sc2C1 ; CC(C)(C)OC(=O)N1Cc2nc(N3C4CCC3CN(C(=O)OCc3ccccc3)C4)sc2C1 ; O=C(OCc1ccccc1)N1CC2CCC(C1)N2c1nc2c(s1)CNC2]

A solution of di-tert-butyl dicarbonate (0.30 mL, 1.28 mmol) in acetonitrile (2 mL) was added dropwise to a solution of 2-bromo-5,6-dihydro-4H-pyrrolo[3,4-d]thiazole hydrobromide (244 mg, 0.85 mmol) and 4-dimethylaminopyridine (136 mg, 1.11 mmol) in acetonitrile (2 mL). The mixture was stirred at room temperature for 1 hour. The mixture was poured into water (30 mL) and the suspension was stirred for 30 minutes. The precipitate was filtered off, washed with water and air dried. The solid was redissolved in dichloromethane, dried with sodium sulfate and concentrated in vacuo to afford tert-butyl 2-bromo-4,6-dihydro-5H-pyrrolo[3,4-d]thiazole-5-carboxylate (216 mg, 82%) as a grey solid.

Under an argon atmosphere, RuPhos (57.8 mg, 0.12 mmol) and Pd₂(dba)₃ (50.4 mg, 0.06 mmol) were suspended in 1,4-dioxane (0.5 mL) and heated to 80° C. for 5 minutes. The mixture was cooled to room temperature and transferred under an argon atmosphere to a stirring solution of tert-butyl 2-bromo-4,6-dihydro-5H-pyrrolo[3,4-d]thiazole-5-carboxylate (210 mg, 0.69 mmol), benzyl 3,8-diazabicyclo[3.2.1]octane-3-carboxylate (288 mg, 1.17 mmol, Intermediate 1) and cesium carbonate (673 mg, 2.06 mmol) in 1,4-dioxane (4 mL). The mixture was heated to 100° C., stirred for 20 hours and concentrated in vacuo. The residue was partitioned between ethyl acetate (15 mL) and water (10 mL) and the layers were separated. The organic layer was washed with brine, dried with sodium sulfate and concentrated in vacuo to afford a brown oil. The crude oil was purified with silica column chromatography (0% to 70% ethyl acetate in n-heptane) and concentrated in vacuo to afford tert-butyl 2-(3-((benzyloxy)carbonyl)-3,8-diazabicyclo[3.2.1]octan-8-yl)-4,6-dihydro-5H-pyrrolo[3,4-d]thiazole-5-carboxylate (177 mg, 54%) as a white foam.

To a solution tert-butyl 2-(3-((benzyloxy)carbonyl)-3,8-diazabicyclo[3.2.1]octan-8-yl)-4,6-dihydro-5H-pyrrolo[3,4-d]thiazole-5-carboxylate (162 mg, 0.34 mmol) in dichloromethane (1 mL) was added trifluoroacetic acid (0.53 mL, 6.89 mmol) and the mixture was stirred at room temperature for 2 hours. The mixture was poured into saturated aqueous sodium carbonate solution (pH >10) and extracted with dichloromethane three times. The combined organic layers were washed with brine, dried with sodium sulfate and concentrated in vacuo to afford benzyl 8-(5,6-dihydro-4H-pyrrolo[3,4-d]thiazol-2-yl)-3,8-diazabicyclo[3.2.1]octane-3-carboxylate (138 mg, 95%, Intermediate 3). LCMS (Method B): t_{R} 1.69 min, 95%, MS (ESI) 371.1 (M+H)⁺.

### Intermediate 5: Synthesis of benzyl 8-(4,5,6,7-tetrahydropyrazolo[1,5-a]pyrazin-2-yl)-3,8-diazabicyclo[3.2.1]octane-3-carboxylate

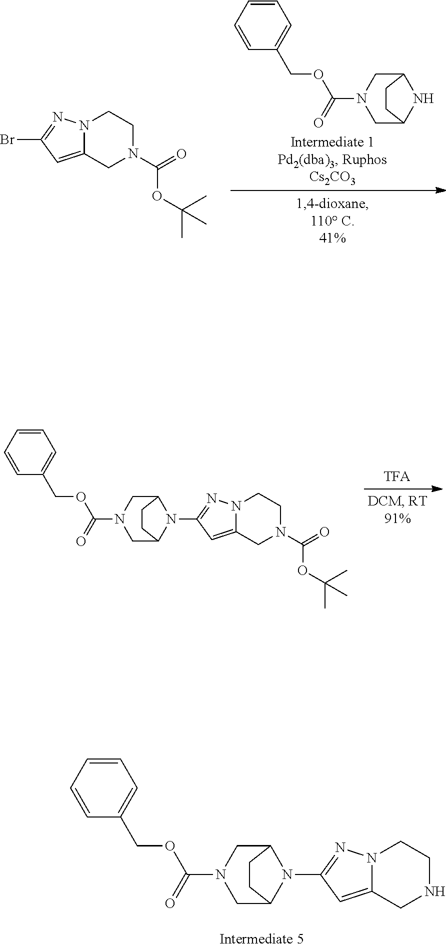 [SMILES: CC(C)(C)OC(=O)N1CCn2nc(Br)cc2C1 ; O=C(OCc1ccccc1)N1CC2CCC(C1)N2 ; CC(C)(C)OC(=O)N1CCn2nc(N3C4CCC3CN(C(=O)OCc3ccccc3)C4)cc2C1 ; O=C(OCc1ccccc1)N1CC2CCC(C1)N2c1cc2n(n1)CCNC2]

Under an argon atmosphere, RuPhos (265 mg, 0.57 mmol) and Pd₂(dba)₃ (236 mg, 0.26 mmol) were suspended in 1,4-dioxane (2 mL) and heated to 80° C. for 5 minutes. The mixture was cooled to room temperature and transferred under an argon atmosphere to a stirring solution of tert-butyl 2-bromo-6,7-dihydropyrazolo[1,5-a]pyrazine-5(4H)-carboxylate (779 mg, 2.58 mmol), benzyl 3,8-diazabicyclo[3.2.1]octane-3-carboxylate (889 mg, 3.61 mmol, Intermediate 1) and cesium carbonate (2.52 g, 7.73 mmol) in 1,4-dioxane (7 mL) and the mixture was heated to 110° C. for 16 hours. The mixture was filtered through Celite®, washing with acetonitrile and concentrated in vacuo. The residue was purified with silica column chromatography (0% to 100% ethyl acetate in n-heptane) and concentrated in vacuo to afford tert-butyl 2-(3-((benzyloxy)carbonyl)-3,8-diazabicyclo[3.2.1]octan-8-yl)-6,7-dihydropyrazolo[1,5-a]pyrazine-5(4H)-carboxylate (494 mg, 41%) as an orange solid. To a solution of tert-butyl 2-(3-((benzyloxy)carbonyl)-3,8-diazabicyclo[3.2.1]octan-8-yl)-6,7-dihydropyrazolo[1,5-a]pyrazine-5(4H)-carboxylate (494 mg, 1.06 mmol) in dichloromethane (2 mL) was added trifluoroacetic acid (1.63 mL, 21.1 mmol) and the mixture was stirred at room temperature for 45 minutes. The mixture was concentrated in vacuo, redissolved in dichloromethane (10 mL) and basified with aqueous potassium carbonate solution. The biphasic mixture was extracted with dichloromethane three times. The combined organic layers were washed with brine, concentrated in vacuo and co-evaporated with n-heptane to afford benzyl 8-(4,5,6,7-tetrahydropyrazolo[1,5-a]pyrazin-2-yl)-3,8-diazabicyclo[3.2.1]octane-3-carboxylate (354 mg, 89%, Intermediate 5) as an orange solid. LCMS (Method B): t_{R} 1.77 min, 97%, MS (ESI) 368.2 (M+H)⁺.

### Intermediate 6: Synthesis of tert-butyl 8-carbamothioyl-3,8-diazabicyclo[3.2.1]octane-3-carboxylate

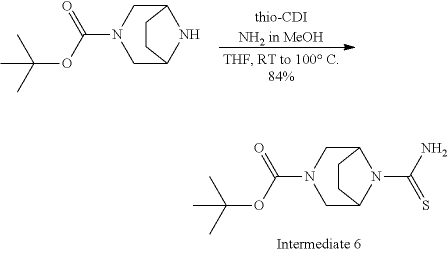 [SMILES: CC(C)(C)OC(=O)N1CC2CCC(C1)N2 ; CC(C)(C)OC(=O)N1CC2CCC(C1)N2C(N)=S]

To a solution of tert-butyl 3,8-diazabicyclo[3.2.1]octane-3-carboxylate (450 mg, 2.12 mmol) in tetrahydrofuran (7 mL) was added 1,1′-thiocarbonyldiimidazole (529 mg, 2.97 mmol) and the mixture was stirred at room temperature for 6 hours. Next, 7 N ammonia in methanol (9 mL, 63 mmol) was added and the mixture was stirred at 100° C. in a sealed vial for 16 hours. The mixture was concentrated in vacuo and the residue was partitioned between 10% aqueous citric acid solution and ethyl acetate. The organic layer was washed with 10% aqueous citric acid solution followed by water, saturated aqueous sodium bicarbonate solution and brine. The organic layer was dried with sodium sulfate and concentrated in vacuo to afford tert-butyl 8-carbamothioyl-3,8-diazabicyclo[3.2.1]octane-3-carboxylate (488 mg, 84%, Intermediate 6) as an orange foam. LCMS (Method B): t_{R} 1.73 min, 98%, MS (ESI) 272.1 (M+H)⁺.

### Intermediate 7: Synthesis of 5-bromo-3,3-dimethyl-1-(2,2,2-trifluoroacetyl) piperidin-4-one

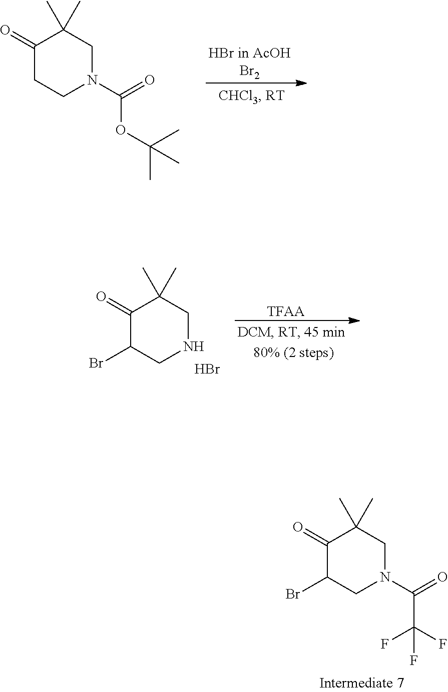

To a solution of tert-butyl 3,3-dimethyl-4-oxopiperidine-1-carboxylate (750 mg, 3.30 mmol) in chloroform (15 mL) was added 33% hydrogen bromide in acetic acid (0.57 mL, 3.30 mmol) and the mixture was stirred at room temperature for 5 minutes. Next, a solution of bromine (0.17 mL, 3.30 mmol) in chloroform (3 mL) was added and the mixture was stirred at room temperature for 30 minutes. The mixture was concentrated in vacuo, the residue was suspended in a mixture of triethylamine (1.80 mL, 12.9 mmol) and dichloromethane (15 mL) and taken forward into the next step without further purification.

To the above solution was slowly added trifluoroacetic anhydride (0.70 mL, 5.03 mmol) and the mixture was stirred at room temperature for 45 minutes. The mixture was concentrated, and the residue was partitioned between water and ethyl acetate. The organic layer was washed with brine, dried with sodium sulfate and concentrated in vacuo to afford 5-bromo-3,3-dimethyl-1-(2,2,2-trifluoroacetyl) piperidin-4-one (1.32 g, 80% over two steps, Intermediate 7) as a light orange solid. ¹H-NMR: δ_{H} (400 MHz, CDCl₃, mixture of rotamers) 4.93-4.84 (0.5H, m), 4.83-4.68 (1H, m), 4.58-4.48 (0.5H, m), 4.40-4.31 (0.5H, m), 3.86-3.68 (1H, m), 3.63-3.48 (1H, m), 3.19-3.10 (0.5H, m), 1.27 (3H, s), 1.25-1.21 (3H, m).

### Intermediate 8: Synthesis of tert-butyl 8-(7,7-dimethyl-4,5,6,7-tetrahydrothiazolo[5,4-c]pyridin-2-yl)-3,8-diazabicyclo[3.2.1]octane-3-carboxylate

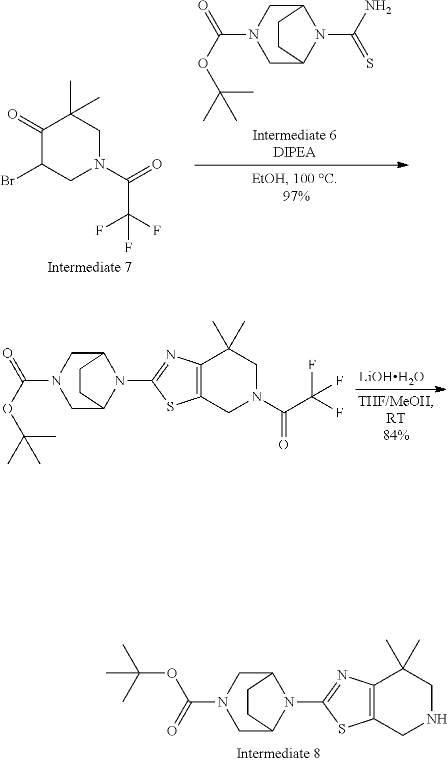

A mixture of tert-butyl 8-carbamothioyl-3,8-diazabicyclo[3.2.1]octane-3-carboxylate (386 mg, 1.42 mmol, Intermediate 6), 5-bromo-3,3-dimethyl-1-(2,2,2-trifluoroacetyl) piperidin-4-one (602 mg, 1.99 mmol, Intermediate 7) and N,N-diisopropylethylamine (2.5 mL, 14.3 mmol) in ethanol (3 mL) was heated to 100° C. for 4 hours. The mixture was concentrated in vacuo, purified with silica column chromatography (0% to 100% ethyl acetate in n-heptane) and concentrated in vacuo to afford 8-(7,7-dimethyl-5-(2,2,2-trifluoroacetyl)-4,5,6,7-tetrahydrothiazolo[5,4-c]pyridin-2-yl)-3,8-diazabicyclo[3.2.1]octane-3-carboxylate (758 mg, 97%) as a yellow oil.

To a solution of tert-butyl 8-(7,7-dimethyl-5-(2,2,2-trifluoroacetyl)-4,5,6,7-tetrahydrothiazolo[5,4-c]pyridin-2-yl)-3,8-diazabicyclo[3.2.1]octane-3-carboxylate (750 mg, 1.58 mmol) in methanol (3 mL) and tetrahydrofuran (3 mL) was added a solution of lithium hydroxide monohydrate (66.3 mg, 1.58 mmol) in water (1.5 mL) and the mixture was stirred at room temperature for 16 hours. The mixture was concentrated, partitioned between water and dichloromethane and the aqueous layer was extracted with dichloromethane twice. The combined organic layers were washed with brine, concentrated in vacuo and co-evaporated with n-heptane to afford tert-butyl 8-(7,7-dimethyl-4,5,6,7-tetrahydrothiazolo[5,4-c]pyridin-2-yl)-3,8-diazabicyclo[3.2.1]octane-3-carboxylate (507 mg, 84%, Intermediate 8) as a yellow solid. LCMS (Method B): t_{R} 2.04 min, 99%, MS (ESI) 379.2 (M+H)⁺.

### Intermediate 9: Synthesis of tert-butyl 8-(4,5,6,7-tetrahydrothiazolo[5,4-c]pyridin-2-yl)-3,8-diazabicyclo[3.2.1]octane-3-carboxylate

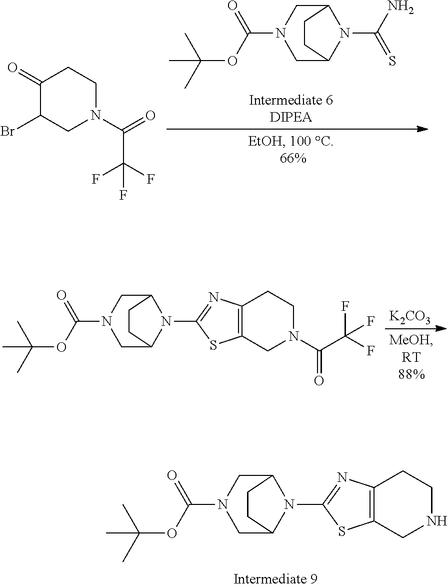

A mixture of tert-butyl 8-carbamothioyl-3,8-diazabicyclo[3.2.1]octane-3-carboxylate (5.50 g, 20.3 mmol, Intermediate 6), 3-bromo-1-(2,2,2-trifluoroacetyl) piperidin-4-one (6.11 g, 22.3 mmol) and N,N-diisopropylethylamine (4.25 mL, 24.3 mmol) in 2-propanol (60 mL) was heated to 70° C. with stirring for 16 hours. The mixture was concentrated in vacuo and partitioned between 10% aqueous citric acid solution and ethyl acetate. The layers were separated, and the organic layer was subsequently washed with water, saturated aqueous sodium bicarbonate and brine. The organic layer was dried with sodium sulfate and coated onto silica. The coated product was purified with silica column chromatography (0% to 100% ethyl acetate in n-heptane) and concentrated in vacuo to afford tert-butyl 8-(5-(2,2,2-trifluoroacetyl)-4,5,6,7-tetrahydrothiazolo[5,4-c]pyridin-2-yl)-3,8-diazabicyclo[3.2.1]octane-3-carboxylate (6.06 g, 66%) as a white solid.

To a solution of tert-butyl 8-(5-(2,2,2-trifluoroacetyl)-4,5,6,7-tetrahydrothiazolo[5,4-c]pyridin-2-yl)-3,8-diazabicyclo[3.2.1]octane-3-carboxylate (6.06 g, 13.6 mmol) in methanol (40 mL) was added potassium carbonate (2.06 g, 14.9 mmol) and the mixture was stirred at room temperature for 2 days. The mixture was concentrated in vacuo, partitioned between water and diethyl ether and acidified with 1M potassium hydrogen sulfate. The aqueous layer was washed with diethyl ether twice, basified with aqueous potassium carbonate to pH >10 and extracted with dichloromethane three times. The combined organic layers were washed with brine, dried with sodium sulfate and concentrated in vacuo to afford butyl 8-(4,5,6,7-tetrahydrothiazolo[5,4-c]pyridin-2-yl)-3,8-diazabicyclo[3.2.1]octane-3-carboxylate (4.22 g, 88%, Intermediate 9) as a light-yellow solid. ¹H-NMR: δ_{H} (400 MHz, CDCl₃, mixture of rotamers) 4.28-4.11 (2H, m), 3.93-3.61 (4H, m), 3.41-3.19 (2H, m), 3.19-3.08 (2H, m), 2.67-2.55 (2H, m), 2.10-1.93 (2H, m), 1.93-1.72 (2H, m), 1.45 (9H, s); LCMS (Method): t_{R} 1.83 min, 98%, MS (ESI) 351.2 (M+H)⁺.

### Intermediate 10: Synthesis of tert-butyl 4-(4,5,6,7-tetrahydrothiazolo[5,4-c]pyridin-2-yl)piperazine-1-carboxylate

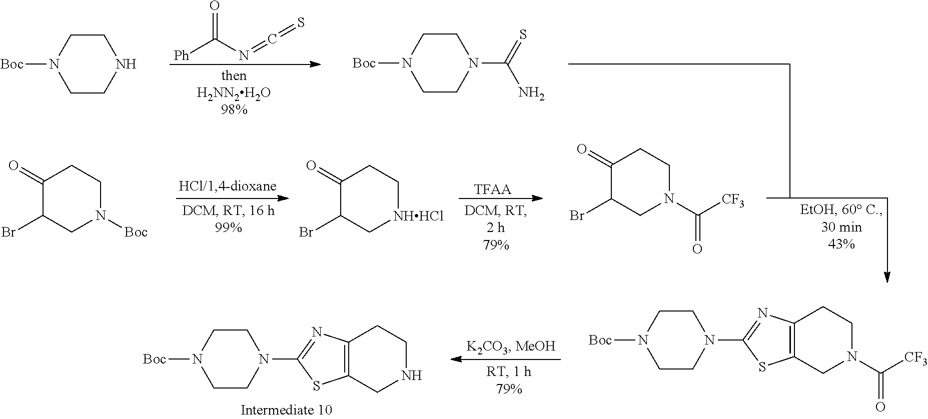

To a stirred solution of tert-butyl piperazine-1-carboxylate (10.0 g, 53.7 mmol) in DCM (50 mL) was added benzoyl isothiocyanate (8.76 g, 53.7 mmol). The mixture was stirred at ambient temperature for 2 h, then concentrated under reduced pressure. To the crude residue was added hydrazine hydrate (13.4 g, 268 mmol) and the mixture was stirred at r.t. for 4 h. The reaction mixture was diluted with water (100 mL) and extracted with DCM (2×100 mL). The combined organic extracts were washed with water (100 mL) and brine (50 mL), dried over anhydrous sodium sulfate and concentrated under reduced pressure. The residue was triturated with hexanes to afford tert-butyl 4-carbamothioylpiperazine-1-carboxylate (13.0 g, 98%) as an off-white solid.

To a stirred solution of tert-butyl 3-bromo-4-oxopiperidine-1-carboxylate (25.0 g, 90.0 mmol) in DCM (50 mL) at 0° C. was added HCl (4 M in dioxane; 45 mL, 180 mmol). The reaction mixture was stirred at r.t. for 16 h, then concentrated under reduced pressure to afford 3-bromopiperidin-4-one hydrochloride (19.0 g, 99%) as an off-white solid.

To a stirred solution of 3-bromopiperidin-4-one hydrochloride (19.5 g, 91.0 mmol) in DCM (200 mL) was added 2,2,2-trifluoroacetic anhydride (63.2 mL, 455 mmol). The mixture was stirred at r.t. for 2 h, then concentrated under reduced pressure. The residue was diluted with water, neutralized with saturated sodium bicarbonate solution, and the organic components were extracted with DCM (2×200 mL). The combined organic extract was washed with brine (100 mL), dried over anhydrous sodium sulfate, filtered and concentrated under reduced pressure to obtain crude material, which was triturated with hexanes to afford 3-bromo-1-(2,2,2-trifluoroacetyl) piperidin-4-one (20 g, 79%) as a brown solid.

To a stirred solution of tert-butyl 4-carbamothioylpiperazine-1-carboxylate (7.50 g, 30.6 mmol) in EtOH (30 mL) was added 3-bromo-1-(2,2,2-trifluoroacetyl) piperidin-4-one (8.38 g, 30.6 mmol). The mixture was heated at 60° C. for 30 minutes, then concentrated under reduced pressure. The residue was triturated with 20% EtOAc in petroleum ether (50 mL) then washed with petroleum ether (50 mL) and dried under vacuum to afford tert-butyl 4-(5-(2,2,2-trifluoroacetyl)-4,5,6,7-tetrahydrothiazolo[5,4-c]pyridin-2-yl)piperazine-1-carboxylate (5.50 g, 43%) as an off-white solid.

To a stirred solution of tert-butyl 4-(5-(2,2,2-trifluoroacetyl)-4,5,6,7-tetrahydrothiazolo[5,4-c]pyridin-2-yl)piperazine-1-carboxylate (11.0 g, 26.2 mmol) in MeOH (100 mL) and water (50 mL) was added potassium carbonate (7.23 g, 52.3 mmol). The mixture was stirred at r.t. for 1 h, then concentrated under reduced pressure, diluted with water (50 mL), and extracted with 10% MeOH in DCM (2×50 mL). Then combined organic layer was washed with water and brine, dried over anhydrous sodium sulfate and concentrated under reduced pressure. The residue was triturated with 10% MeOH in EtOAc to afford tert-butyl 4-(4,5,6,7-tetrahydrothiazolo[5,4-c]pyridin-2-yl)piperazine-1-carboxylate (6.70 g, 79%, Intermediate 10) as a pale yellow solid.

### Intermediate 11: Synthesis of 1-(2-bromo-6,7-dihydrothiazolo[5,4-c]pyridin-5(4H)-yl)-2-cyclopentylethan-1-one

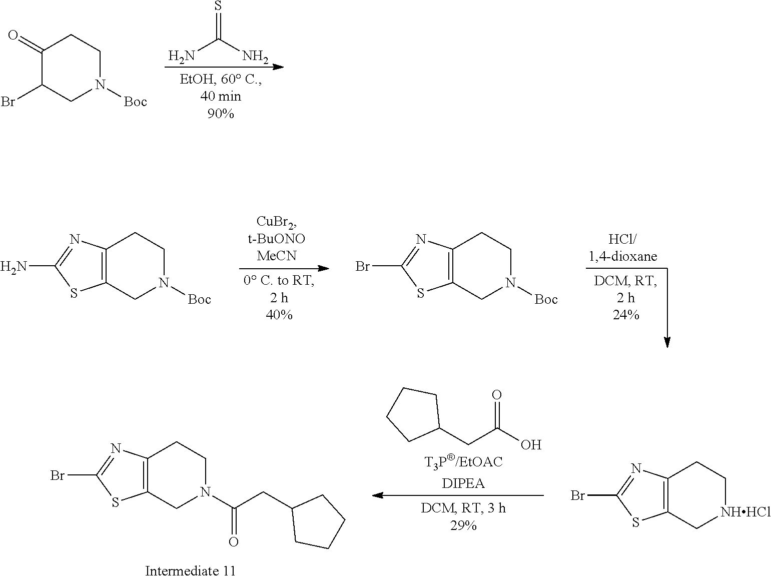

To a stirred solution of thiourea (0.958 g, 12.6 mmol) in EtOH (30 mL) was added tert-butyl 3-bromo-4-oxopiperidine-1-carboxylate (5.0 g, 18.0 mmol). The mixture was heated at 60° C. for 40 minutes, then concentrated under reduced pressure. The residue was triturated with 20% ethyl acetate in petroleum ether and the solid filtered off, washing with petroleum ether and dried under vacuum to afford tert-butyl 2-amino-6,7-dihydrothiazolo[5,4-c]pyridine-5(4H)-carboxylate (4.50 g, 90%).

To a stirred solution of copper (II) bromide (4.55 g, 20.4 mmol) in acetonitrile (25 mL) at 0° C. under nitrogen was added dropwise tert-butyl nitrite (2.10 g, 20.4 mmol). After 20 minutes, tert-butyl 2-amino-6,7-dihydrothiazolo[5,4-c]pyridine-5(4H)-carboxylate (4.00 g, 15.7 mmol) was added. The reaction mixture was stirred at r.t. for 2 h, then diluted with ethyl acetate (100 mL), washed with 1.5 N aqueous HCl (2×10 mL), then water (20 mL) and brine (20 mL), dried over Na₂SO₄ and concentrated under reduced pressure to afford tert-butyl 2-bromo-6,7-dihydrothiazolo[5,4-c]pyridine-5(4H)-carboxylate (3.80 g, 40%).

To a stirred solution of tert-butyl 2-bromo-6,7-dihydrothiazolo[5,4-c]pyridine-5(4H)-carboxylate (5.0 g, 15.7 mmol) in DCM (25 mL) at r.t. was added HCl in 1,4-dioxane (4 M; 7.83 mL, 31.3 mmol). The reaction mixture was stirred at r.t. for 2 h, then concentrated under reduced pressure to afford 2-bromo-4,5,6,7-tetrahydrothiazolo[5,4-c]pyridine hydrochloride (2.70 g, 24%).

To a stirred solution of 2-bromo-4,5,6,7-tetrahydrothiazolo[5,4-c]pyridine hydrochloride (2.80 g, 11.0 mmol) in DCM (25 mL) under nitrogen at r.t. was added 2-cyclopentylacetic acid (2.11 g, 16.4 mmol), n-propylphosphonic acid anhydride, cyclic trimer (50% in ethyl acetate; 3.19 mL, 11.0 mmol) and N,N-diisopropylethylamine (7.08 g, 54.8 mmol). The reaction mixture was stirred at r.t. for 3 h, then diluted with DCM (25 mL), washed with water (20 mL) and brine (20 mL), dried over Na₂SO₄, filtered and concentrated under reduced pressure. The residue was purified by flash chromatography, eluting with 70% ethyl acetate in petroleum ether, to afford 1-(2-bromo-6,7-dihydrothiazolo[5,4-c]pyridin-5(4H)-yl)-2-cyclopentylethan-1-one (1.30 g, 29%, Intermediate 11).

The following intermediates (Intermediates 12-13) were prepared using procedures analogous to Intermediate 8, using the appropriate starting materials.

### Intermediate 12: Synthesis of tert-butyl 8-(5′,6′-dihydro-4′H-spiro[cyclopropane-1,7′-thiazolo[5,4-c]pyridin]-2′-yl)-3,8-diazabicyclo[3.2.1]octane-3-carboxylate

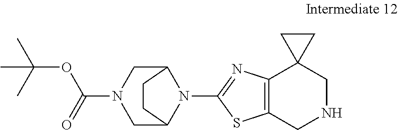 [SMILES: CC(C)(C)OC(=O)N1CC2CCC(C1)N2c1nc2c(s1)CNCC21CC1]

Prepared using tert-butyl 8-oxo-5-azaspiro[2.5]octane-5-carboxylate and Intermediate 6. LCMS (Method B): t_{R} 2.06 min, 100%, MS (ESI) 377.2 (M+H)⁺.

### Intermediate 13: Synthesis of tert-butyl 8-(2,3,5,5′,6,6′-hexahydro-4′H-spiro[pyran-4,7′-thiazolo[5,4-c]pyridin]-2′-yl)-3,8-diazabicyclo[3.2.1]octane-3-carboxylate

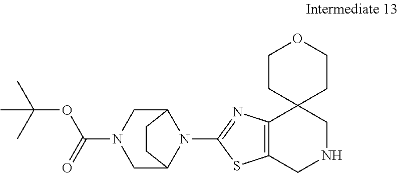 [SMILES: CC(C)(C)OC(=O)N1CC2CCC(C1)N2c1nc2c(s1)CNCC21CCOCC1]

Prepared using 9-oxa-2-azaspiro[5.5]undecan-5-one hydrochloride and Intermediate 6. ¹H-NMR: δ_{H} (400 MHz, CDCl₃, mixture of rotamers) 4.20 (2H, s), 4.13-3.99 (2H, m), 3.90-3.77 (3H, m), 3.75-3.61 (3H, m), 3.33 (1H, d, J 12.8), 3.24 (1H, d, J 12.8), 3.00 (2H, s), 2.19-2.07 (2H, m), 2.07-1.94 (2H, m), 1.91-1.73 (2H, m), 1.53-1.43 (11H, m); LCMS (Method A): t_{R} 1.51 min, 100%, MS (ESI) 421.2 (M+H)⁺.

### Intermediate 14: Synthesis of 2-bromo-6,6-dimethyl-4,5,6,7-tetrahydrothiazolo[5,4-c]pyridine

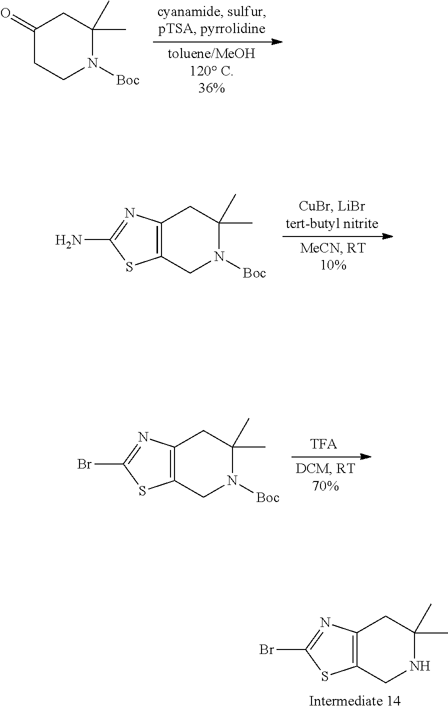 [SMILES: CC(C)(C)OC(=O)N1CCC(=O)CC1(C)C ; CC(C)(C)OC(=O)N1Cc2sc(N)nc2CC1(C)C ; CC(C)(C)OC(=O)N1Cc2sc(Br)nc2CC1(C)C ; CC1(C)Cc2nc(Br)sc2CN1]

A solution of tert-butyl 2,2-dimethyl-4-oxopiperidine-1-carboxylate (1.30 g, 5.72 mmol) and p-toluenesulfonic acid monohydrate (0.05 g, 0.26 mmol) in a mixture of pyrrolidine (1.5 mL, 18.14 mmol) and toluene (10 mL) was refluxed in a Dean-Stark setup for 16 hours. The mixture was concentrated in vacuo and redissolved in methanol (10 mL). Next, sulfur (0.24 g, 7.43 mmol) and cyanamide (433 mg, 10.29 mmol) were added and the mixture was stirred at room temperature for 4 hours. The mixture was coated onto silica and purified with silica column chromatography (0% to 100% ethyl acetate in n-heptane) and concentrated in vacuo to afford tert-butyl 2-amino-6,6-dimethyl-6,7-dihydrothiazolo[5,4-c]pyridine-5(4H)-carboxylate (749 mg, 36%) as an orange liquid. LCMS (Method B): t_{R} 1.89 min, 99%, MS (ESI) 284.2 (M+H)⁺.

A solution of tert-butyl 2-amino-6,6-dimethyl-6,7-dihydrothiazolo[5,4-c]pyridine-5(4H)-carboxylate (695 mg, 2.45 mmol), copper (I) bromide (457 mg, 3.19 mmol) and lithium bromide (319 mg, 3.68 mmol) in acetonitrile (10 mL) was cooled to 0° C. tert-Butyl nitrite (0.42 mL, 3.19 mmol) was added and the mixture was stirred at room temperature for 16 hours. The mixture was partitioned between ethyl acetate (50 mL) and saturated aqueous ammonium chloride solution and the layers were separated. The organic layer was washed with saturated aqueous sodium bicarbonate solution, brine (50 mL) and concentrated in vacuo. The residue was dissolved in dichloromethane, purified with silica column chromatography (0% to 100% ethyl acetate in n-heptane) and concentrated in vacuo to afford tert-butyl 2-bromo-6,6-dimethyl-6,7-dihydrothiazolo[5,4-c]pyridine-5(4H)-carboxylate (106 mg, 10%) as a colorless oil. ¹H-NMR: OH (400 MHz, CDCl₃, mixture of rotamers) 4.59 (2H, d, J 1.6), 2.84 (2H, d, J 1.7), 1.50 (6H, s), 1.47 (9H, s); LCMS (Method B): t_{R} 2.24 min, 91%, MS (ESI) 349.0 (M+H)⁺.

tert-Butyl 2-bromo-6,6-dimethyl-6,7-dihydrothiazolo[5,4-c]pyridine-5(4H)-carboxylate (106 mg, 0.31 mmol) was dissolved in dichloromethane (1 mL) and trifluoroacetic acid (0.35 mL, 4.58 mmol) was added. The mixture was stirred at room temperature for 1 hour and the mixture was concentrated in vacuo. The residue was partitioned between aqueous potassium carbonate and dichloromethane. The aqueous layer was extracted with dichloromethane three times. The combined organic layers were filtered, concentrated in vacuo and coevaporated with heptane to afford 2-bromo-6,6-dimethyl-4,5,6,7-tetrahydrothiazolo[5,4-c]pyridine (57 mg, 70%, Intermediate 14) as a colorless oil. LCMS (Method B): t_{R} 1.63 min, 95%, MS (ESI) 249.0 (M+H)⁺.

The following intermediate (Intermediate 15) was prepared using procedures analogous to Intermediate 14, using the appropriate starting materials.

### Intermediate 15: Synthesis of tert-butyl 2-bromo-7,7-difluoro-6,7-dihydrothiazolo[5,4-c]pyridine-5(4H)-carboxylate

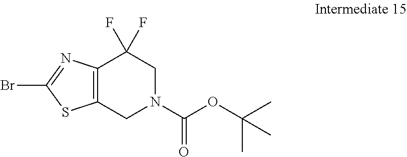 [SMILES: CC(C)(C)OC(=O)N1Cc2sc(Br)nc2C(F)(F)C1]

Prepared using tert-butyl 3,3-difluoro-4-oxopiperidine-1-carboxylate. ¹H-NMR: δ_{H} (400 MHz, CDCl₃) 4.70 (2H, s), 4.06 (2H, t, J 10.5), 1.49 (9H, s); LCMS (Method B): t_{R} 2.09 min, 99%, MS (ESI) 355.0 (M+H)⁺.

### Synthetic Procedures for Final Compounds:

### Example 1: Synthesis of 1-(2-(3,8-diazabicyclo[3.2.1]octan-8-yl)-6,7-dihydrothiazolo[5,4-c]pyridin-5(4H)-yl)-2-cyclopentylethan-1-one

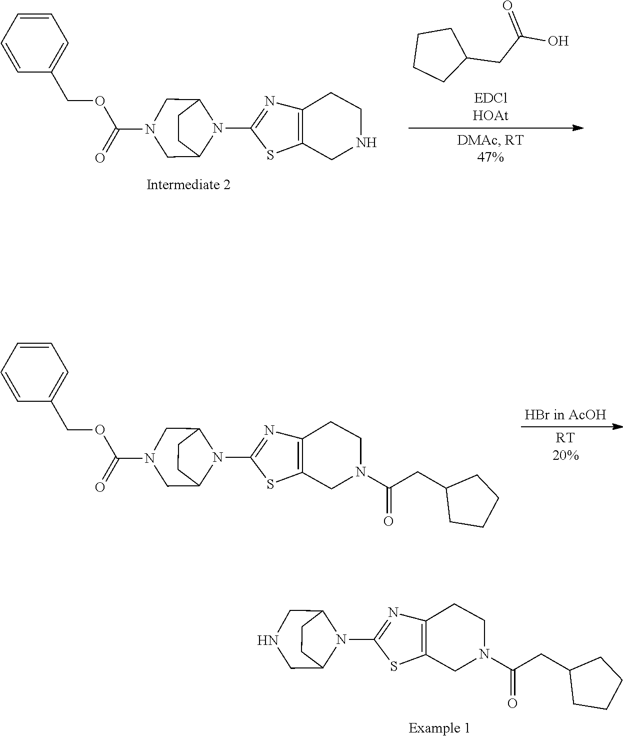 [SMILES: O=C(OCc1ccccc1)N1CC2CCC(C1)N2c1nc2c(s1)CNCC2 ; O=C(O)CC1CCCC1 ; O=C(CC1CCCC1)N1CCc2nc(N3C4CCC3CN(C(=O)OCc3ccccc3)C4)sc2C1 ; O=C(CC1CCCC1)N1CCc2nc(N3C4CCC3CNC4)sc2C1]

A solution of benzyl 8-(4,5,6,7-tetrahydrothiazolo[5,4-c]pyridin-2-yl)-3,8-diazabicyclo[3.2.1]octane-3-carboxylate (150 mg, 0.39 mmol, Intermediate 2), 2-cyclopentylacetic acid (55.0 mg, 0.43 mmol), N-(3-dimethylaminopropyl)-N′-ethylcarbodiimide hydrochloride (90 mg, 0.47 mmol) and 1-hydroxy-7-azabenzotriazole (26.6 mg, 0.20 mmol) in N,N-dimethylacetamide (3 mL) was stirred at room temperature for 16 hours. The crude mixture was purified with preparative reversed phase chromatography (Phenomenex LUNA C18 column, formic acid in water/acetonitrile) and lyophilized to afford benzyl 8-(5-(2-cyclopentylacetyl)-4,5,6,7-tetrahydrothiazolo[5,4-c]pyridin-2-yl)-3,8-diazabicyclo[3.2.1]octane-3-carboxylate (95 mg, 47%) as a white solid.

A solution of benzyl 8-(5-(2-cyclopentylacetyl)-4,5,6,7-tetrahydrothiazolo[5,4-c]pyridin-2-yl)-3,8-diazabicyclo[3.2.1]octane-3-carboxylate (95 mg, 0.18 mmol) in 33% hydrogen bromide in acetic acid (5 mL, 87 mmol) was stirred at room temperature for 2 hours. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-cyclopentylethan-1-one (15 mg, 20%, Example 1) as a white solid. ¹H-NMR: δ_{H} (400 MHz, CDCl₃, mixture of rotamers) 6.10 (1H, s), 4.57-4.43 (2H, m), 4.19-3.92 (2H, m), 3.77-3.67 (2H, m), 3.07-2.89 (2H, m), 2.62-2.52 (2H, m), 2.44-2.35 (2H, m), 2.24-2.06 (2H, m), 1.95-1.81 (4H, m), 1.79-1.64 (3H, m), 1.62-1.43 (4H, m), 1.18-1.05 (2H, m); UPLC (Method A): t_{R} 1.50 min, 100%, MS (ESI) 361.2 (M+H)⁺.

The following examples (2-29) were prepared using procedures analogous to Example 1, using the appropriate starting materials.

### Example 2: Synthesis of 1-(2-(3,8-diazabicyclo[3.2.1]octan-8-yl)-6,7-dihydrothiazolo[5,4-c]pyridin-5(4H)-yl)-2-(4,4-difluorocyclohexyl)ethan-1-one

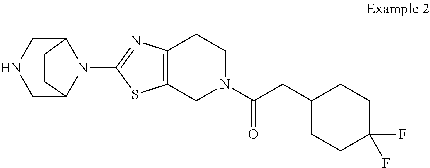 [SMILES: O=C(CC1CCC(F)(F)CC1)N1CCc2nc(N3C4CCC3CNC4)sc2C1]

Prepared using 2-(4,4-difluorocyclohexyl)acetic acid as the acid component.

¹H-NMR: δ_{H} (400 MHz, CDCl₃, mixture of rotamers) 4.66-4.45 (2H, m), 4.17-4.08 (2H, m), 3.93-3.70 (2H, m), 3.22 (2H, d, J 12.2), 2.76-2.64 (4H, m), 2.38-2.28 (2H, m), 2.12-1.65 (12H, m), 1.40-1.22 (2H, m); UPLC (Method C): t_{R} 4.06 min, 98%, MS (ESI) 411.2 (M+H)⁺.

### Example 3: Synthesis of 1-(2-(3,8-diazabicyclo[3.2.1]octan-8-yl)-6,7-dihydrothiazolo[5,4-c]pyridin-5(4H)-yl)-2-(tetrahydro-2H-pyran-4-yl)ethan-1-one

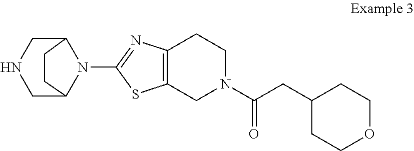 [SMILES: O=C(CC1CCOCC1)N1CCc2nc(N3C4CCC3CNC4)sc2C1]

Prepared using 2-(tetrahydro-2H-pyran-4-yl)acetic acid as the acid component.

UPLC (Method B): t_{R} 0.90 min, 97%, MS (ESI) 377.2 (M+H)⁺.

### Example 4: Synthesis of 1-(2-(3,8-diazabicyclo[3.2.1]octan-8-yl)-6,7-dihydrothiazolo[5,4-c]pyridin-5(4H)-yl)-2-isopropoxyethan-1-one

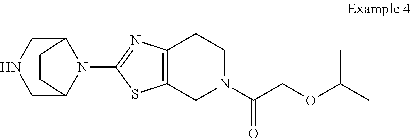 [SMILES: CC(C)OCC(=O)N1CCc2nc(N3C4CCC3CNC4)sc2C1]

Prepared using 2-isopropoxyacetic acid as the acid component.

¹H-NMR: δ_{H} (400 MHz, DMSO-d₆, mixture of rotamers) 4.57-3.92 (6H, m), 3.78-3.65 (2H, m), 3.65-3.54 (1H, m), 2.92 (3H, d, J 12.1), 2.71-2.57 (2H, m), 2.54-2.51 (2H, m), 1.97-1.78 (4H, m), 1.15-1.01 (6H, m); UPLC (Method B): t_{R} 0.95 min, 93%, MS (ESI) 351.2 (M+H)⁺.

### Example 5: Synthesis of 1-(2-(3,8-diazabicyclo[3.2.1]octan-8-yl)-6,7-dihydrothiazolo[5,4-c]pyridin-5(4H)-yl)-3-phenylpropan-1-one

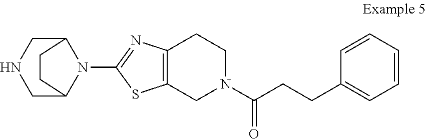 [SMILES: O=C(CCc1ccccc1)N1CCc2nc(N3C4CCC3CNC4)sc2C1]

Prepared using 3-phenylpropanoic acid as the acid component.

¹H-NMR: δ_{H} (400 MHz, DMSO-d₆, mixture of rotamers) 7.32-7.10 (6H, m), 4.81-4.46 (2H, m), 4.14-3.94 (2H, m), 3.79-3.66 (2H, m), 3.01-2.64 (8H, m), 1.94-1.80 (4H, m); UPLC (Method B): t_{R} 1.19 min, 100%, MS (ESI) 383.2 (M+H)⁺.

### Example 6: Synthesis of 1-(2-(3,8-diazabicyclo[3.2.1]octan-8-yl)-6,7-dihydrothiazolo[5,4-c]pyridin-5(4H)-yl)-2-phenoxyethan-1-one

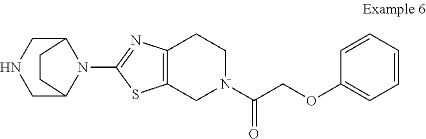 [SMILES: O=C(COc1ccccc1)N1CCc2nc(N3C4CCC3CNC4)sc2C1]

Prepared using 2-phenoxyacetic acid as the acid component.

UPLC (Method B): t_{R} 1.13 min, 97%, MS (ESI) 385.1 (M+H)⁺.

### Example 7: Synthesis of 4-(3-(2-(3,8-diazabicyclo[3.2.1]octan-8-yl)-6,7-dihydrothiazolo[5,4-c]pyridin-5(4H)-yl)-3-oxopropyl)benzonitrile

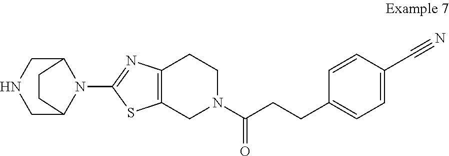 [SMILES: N#Cc1ccc(CCC(=O)N2CCc3nc(N4C5CCC4CNC5)sc3C2)cc1]

Prepared using 3-(4-cyanophenyl) propanoic acid as the acid component.

¹H-NMR: δ_{H} (400 MHz, DMSO-d₆, mixture of rotamers) 7.82-7.63 (2H, m), 7.55-7.35 (2H, m), 4.79-4.70 (1H, m), 4.55-4.45 (1H, m), 3.98 (1H, s), 3.83-3.65 (2H, m), 2.98-2.88 (3H, m), 2.88-2.71 (3H, m), 2.61-2.51 (4H, m), 1.94-1.80 (2H, m); UPLC (Method B): t_{R} 1.12 min, 99%, MS (ESI) 408.1 (M+H)⁺.

### Example 8: Synthesis of 1-(2-(3,8-diazabicyclo[3.2.1]octan-8-yl)-6,7-dihydrothiazolo[5,4-c]pyridin-5(4H)-yl)-2-phenylethan-1-one

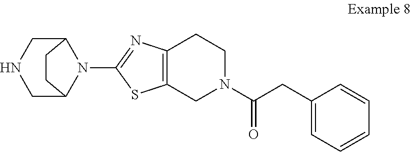 [SMILES: O=C(Cc1ccccc1)N1CCc2nc(N3C4CCC3CNC4)sc2C1]

Prepared using 2-phenylacetic acid as the acid component.

¹H-NMR: δ_{H} (400 MHz, DMSO-d₆, mixture of rotamers) 7.37-7.13 (5H, m), 4.85-4.43 (2H, m), 4.00-3.70 (5H, m), 2.96-2.68 (2H, m), 2.50-2.38 (6H, m), 1.92-1.80 (4H, m); UPLC (Method B): t_{R} 1.11 min, 95%, MS (ESI) 369.1 (M+H)⁺.

### Example 9: Synthesis of 1-(2-(3,8-diazabicyclo[3.2.1]octan-8-yl)-6,7-dihydrothiazolo[5,4-c]pyridin-5(4H)-yl)-3-methylbutan-1-one

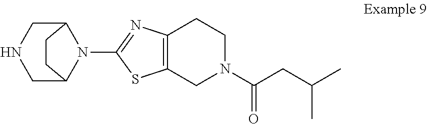 [SMILES: CC(C)CC(=O)N1CCc2nc(N3C4CCC3CNC4)sc2C1]

Prepared using 3-methylbutanoic acid as the acid component.

¹H-NMR: δ_{H} (400 MHz, DMSO-d₆, mixture of rotamers) 4.60-4.44 (2H, m), 4.16-3.94 (2H, m), 3.85-3.64 (2H, m), 3.02-2.84 (2H, m), 2.70-2.55 (4H, m), 2.35-2.20 (2H, m), 2.10-1.64 (2H, m), 1.05-0.81 (6H, m); UPLC (Method B): t_{R} 0.69 min, 96%, MS (ESI) 335.1 (M+H)⁺.

### Example 10: Synthesis of 1-(2-(3,8-diazabicyclo[3.2.1]octan-8-yl)-6,7-dihydrothiazolo[5,4-c]pyridin-5(4H)-yl)-3,3-dimethylbutan-1-one

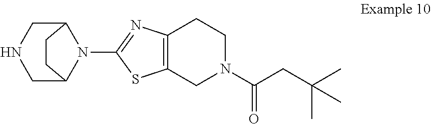 [SMILES: CC(C)(C)CC(=O)N1CCc2nc(N3C4CCC3CNC4)sc2C1]

Prepared using 3,3-dimethylbutanoic acid as the acid component.

¹H-NMR: δ_{H} (400 MHz, DMSO-d₆, mixture of rotamers) 4.88-4.44 (2H, m), 3.98 (2H, s), 3.88-3.69 (2H, m), 2.96-2.72 (2H, m), 2.62-2.51 (4H, m), 2.36-2.25 (3H, m), 1.94-1.79 (3H, m), 1.04-0.90 (9H, m); UPLC (Method B): t_{R} 1.14 min, 95%, MS (ESI) 349.2 (M+H)⁺.

### Example 11: Synthesis of 1-(2-(3,8-diazabicyclo[3.2.1]octan-8-yl)-6,7-dihydrothiazolo[5,4-c]pyridin-5(4H)-yl)-2-cyclobutylethan-1-one

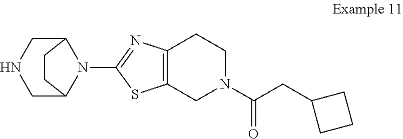 [SMILES: O=C(CC1CCC1)N1CCc2nc(N3C4CCC3CNC4)sc2C1]

Prepared using 2-cyclobutylacetic acid as the acid component.

¹H-NMR: δ_{H} (400 MHz, DMSO-d₆, mixture of rotamers) 4.55-4.41 (2H, m), 4.17-3.94 (2H, m), 3.81-3.64 (2H, m), 3.07-2.87 (2H, m), 2.67-2.52 (5H, m), 2.05 (3H, m), 1.94-1.73 (6H, m), 1.72-1.57 (3H, m); UPLC (Method B): t_{R} 0.73 min, 93%, MS (ESI) 347.1 (M+H)⁺.

### Example 12: Synthesis of 1-(2-(3,8-diazabicyclo[3.2.1]octan-8-yl)-6,7-dihydrothiazolo[5,4-c]pyridin-5(4H)-yl)-2-(bicyclo[1.1.1]pentan-1-yl)ethan-1-one

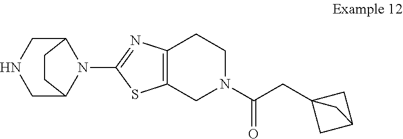 [SMILES: O=C(CC12CC(C1)C2)N1CCc2nc(N3C4CCC3CNC4)sc2C1]

Prepared using 2-(bicyclo[1.1.1]pentan-1-yl)acetic acid as the acid component. ¹H-NMR: δ_{H} (400 MHz, DMSO-d₆, mixture of rotamers) 4.77-4.41 (2H, m), 4.21-3.92 (2H, m), 3.78-3.63 (3H, m), 3.07-2.89 (2H, m), 2.62-2.53 (2H, m), 2.47-2.40 (2H, m), 1.97-1.80 (4H, m), 1.79-1.61 (8H, m); UPLC (Method B): t_{R} 0.74 min, 94%, MS (ESI) 359.2 (M+H)⁺.

### Example 13: Synthesis of 1-(2-(3,8-diazabicyclo[3.2.1]octan-8-yl)-6,7-dihydrothiazolo[5,4-c]pyridin-5(4H)-yl)-2-cyclohexylethan-1-one

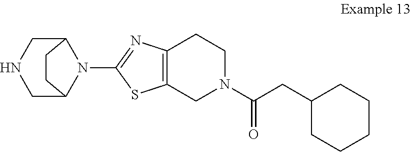 [SMILES: O=C(CC1CCCCC1)N1CCc2nc(N3C4CCC3CNC4)sc2C1]

Prepared using 2-cyclohexylacetic acid as the acid component.

¹H-NMR: δ_{H} (400 MHz, DMSO-d₆, mixture of rotamers) 4.57-4.43 (2H, m), 3.98 (2H, s), 3.82-3.67 (2H, m), 2.92 (2H, d, J 12.3), 2.64-2.55 (2H, m), 2.34-2.18 (2H, m), 1.99-1.79 (4H, m), 1.77-1.54 (7H, m), 1.33-0.85 (6H, m); UPLC (Method B): t_{R} 1.29 min, 94%, MS (ESI) 375.2 (M+H)⁺.

### Example 14: Synthesis of (2-(3,8-diazabicyclo[3.2.1]octan-8-yl)-6,7-dihydrothiazolo[5,4-c]pyridin-5(4H)-yl)(4,4-difluorocyclohexyl)methanone

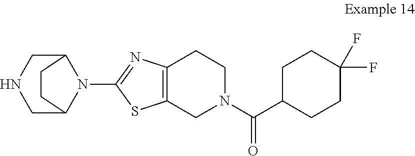 [SMILES: O=C(C1CCC(F)(F)CC1)N1CCc2nc(N3C4CCC3CNC4)sc2C1]

Prepared using 4,4-difluorocyclohexane-1-carboxylic acid as the acid component.

¹H-NMR: δ_{H} (400 MHz, DMSO-d₆, mixture of rotamers) 4.96-4.40 (2H, m), 3.98 (1H, s), 3.92-3.67 (2H, m), 3.00-2.81 (4H, m), 2.66-2.55 (4H, m), 2.13-1.46 (12H, m); UPLC (Method B): t_{R} 1.14 min, 97%, MS (ESI) 397.2 (M+H)⁺.

### Example 15: Synthesis of (2S)-1-(2-(3,8-diazabicyclo[3.2.1]octan-8-yl)-6,7-dihydrothiazolo[5,4-c]pyridin-5(4H)-yl)-2-methylbutan-1-one

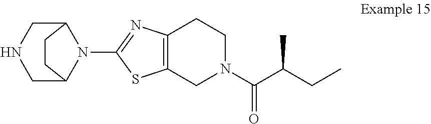 [SMILES: CC[C@H](C)C(=O)N1CCc2nc(N3C4CCC3CNC4)sc2C1]

Prepared using(S)-2-methylbutanoic acid as the acid component.

¹H-NMR: δ_{H} (400 MHz, CDCl₃, mixture of rotamers) 4.71-4.51 (2H, m), 4.16 (2H, s), 3.99-3.74 (2H, m), 3.27 (2H, d, J 12.3), 2.79-2.56 (4H, m), 2.16-1.97 (4H, m), 1.77-1.71 (2H, m), 1.53-1.37 (1H, m), 1.21-1.07 (3H, m), 0.97-0.84 (3H, m); UPLC (Method B): t_{R} 0.69 min, 95%, MS (ESI) 335.1 (M+H)⁺.

### Example 16: Synthesis of (2R)-1-(2-(3,8-diazabicyclo[3.2.1]octan-8-yl)-6,7-dihydrothiazolo[5,4-c]pyridin-5(4H)-yl)-2-methylbutan-1-one

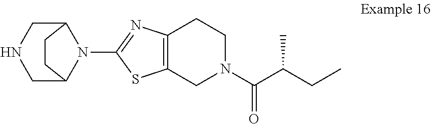 [SMILES: CC[C@@H](C)C(=O)N1CCc2nc(N3C4CCC3CNC4)sc2C1]

Prepared using (R)-2-methylbutanoic acid as the acid component.

¹H-NMR: δ_{H} (400 MHz, CDCl₃, mixture of rotamers) 4.68-4.53 (2H, m), 4.25 (2H, s), 3.97-3.75 (2H, m), 3.38 (2H, d, J 2.4), 2.99 (2H, d, J 12.6), 2.76-2.60 (4H, m), 1.82-1.68 (2H, m), 1.53-1.36 (2H, m), 1.19-1.08 (3H, m), 0.97-0.85 (4H, m); UPLC (Method B): t_{R} 1.28 min, 93%, MS (ESI) 335.2 (M+H)⁺.

### Example 17: Synthesis of 1-(2-(3,8-diazabicyclo[3.2.1]octan-8-yl)-6,7-dihydrothiazolo[5,4-c]pyridin-5(4H)-yl)-2,3-dimethylbutan-1-one

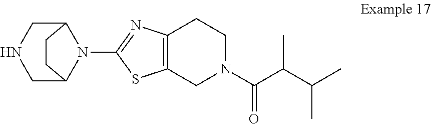 [SMILES: CC(C)C(C)C(=O)N1CCc2nc(N3C4CCC3CNC4)sc2C1]

Prepared using 2,3-dimethylbutanoic acid as the acid component.

¹H-NMR: δ_{H} (400 MHz, CDCl₃, mixture of rotamers) 4.67-4.53 (2H, m), 4.14 (2H, s), 4.03-3.75 (2H, m), 3.24 (2H, d, J 12.2), 2.78-2.64 (4H, m), 2.58-2.41 (1H, m), 2.17-1.86 (5H, m), 1.18-0.84 (9H, m); UPLC (Method B): t_{R} 0.75 min, 90%, MS (ESI) 349.2 (M+H)⁺.

### Example 18: Synthesis of (2S)-1-(2-(3,8-diazabicyclo[3.2.1]octan-8-yl)-6,7-dihydrothiazolo[5,4-c]pyridin-5(4H)-yl)-2,3-dimethylbutan-1-one

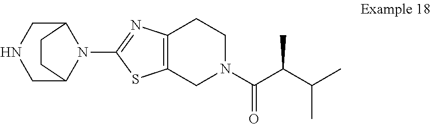 [SMILES: CC(C)[C@H](C)C(=O)N1CCc2nc(N3C4CCC3CNC4)sc2C1]

Prepared using(S)-2,3-dimethylbutanoic acid as the acid component.

¹H-NMR: δ_{H} (400 MHz, CDCl₃, mixture of rotamers) 4.71-4.49 (2H, m), 4.14 (2H, s), 3.98-3.75 (2H, m), 3.23 (2H, d, J 12.3), 2.81-2.61 (4H, m), 2.59-2.41 (1H, m), 2.14-1.84 (5H, m), 1.19-1.04 (3H, m), 1.01-0.83 (6H, m); UPLC (Method A): t_{R} 1.41 min, 100%, MS (ESI) 349.2 (M+H)⁺.

### Example 19: Synthesis of (2R)-1-(2-(3,8-diazabicyclo[3.2.1]octan-8-yl)-6,7-dihydrothiazolo[5,4-c]pyridin-5(4H)-yl)-2,3-dimethylbutan-1-one

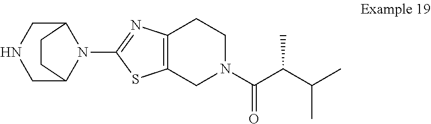 [SMILES: CC(C)[C@@H](C)C(=O)N1CCc2nc(N3C4CCC3CNC4)sc2C1]

Prepared using (R)-2,3-dimethylbutanoic acid as the acid component.

¹H-NMR: δ_{H} (400 MHz, CDCl₃, mixture of rotamers) 4.69-4.50 (2H, m), 4.13 (2H, s), 4.02-3.67 (2H, m), 3.22 (2H, d, J 12.3), 2.76-2.59 (4H, m), 2.60-2.38 (1H, m), 2.15-1.85 (5H, m), 1.19-1.06 (3H, m), 1.00-0.83 (6H, m); UPLC (Method A): t_{R} 1.41 min, 100%, MS (ESI) 349.2 (M+H)⁺.

### Example 20: Synthesis of 1-(2-(3,8-diazabicyclo[3.2.1]octan-8-yl)-6,7-dihydrothiazolo[5,4-c]pyridin-5(4H)-yl)-2-methylpropan-1-one

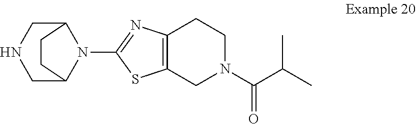 [SMILES: CC(C)C(=O)N1CCc2nc(N3C4CCC3CNC4)sc2C1]

Prepared using isobutyric acid as the acid component.

¹H-NMR: δ_{H} (400 MHz, CDCl₃, mixture of rotamers) 4.69-4.49 (2H, m), 4.15 (2H, s), 3.97-3.73 (2H, m), 3.30-3.06 (2H, m), 2.96-2.78 (1H, m), 2.77-2.65 (3H, m), 2.17-1.93 (4H, m), 1.25-1.06 (7H, m); UPLC (Method B): t_{R} 0.96 min, 92%, MS (ESI) 321.1 (M+H)⁺.

### Example 21: Synthesis of 1-(2-(3,8-diazabicyclo[3.2.1]octan-8-yl)-6,7-dihydrothiazolo[5,4-c]pyridin-5(4H)-yl)-2-(4-fluorophenoxy)ethan-1-one

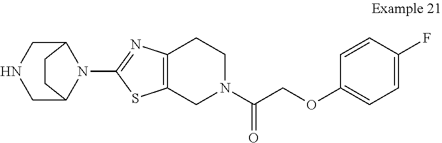 [SMILES: O=C(COc1ccc(F)cc1)N1CCc2nc(N3C4CCC3CNC4)sc2C1]

Prepared using 2-(4-fluorophenoxy)acetic acid as the acid component.

¹H-NMR: δ_{H} (400 MHz, CDCl₃, mixture of rotamers) 7.03-6.94 (2H, m), 6.94-6.85 (2H, m), 4.73 (2H, d, J 10.0), 4.67-4.55 (2H, m), 4.12 (2H, s), 3.89-3.83 (2H, m), 3.21 (2H, d, J 12.6), 2.80-2.58 (4H, m), 2.14-1.98 (2H, m), 1.94-1.91 (2H, m); UPLC (Method C): t_{R} 4.00 min, 96%, MS (ESI) 403.2 (M+H)⁺.

### Example 22: Synthesis of (2-(3,8-diazabicyclo[3.2.1]octan-8-yl)-6,7-dihydrothiazolo[5,4-c]pyridin-5(4H)-yl)(cyclopentyl)methanone

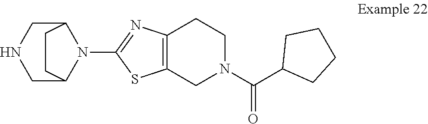 [SMILES: O=C(C1CCCC1)N1CCc2nc(N3C4CCC3CNC4)sc2C1]

Prepared using cyclopentanecarboxylic acid as the acid component.

¹H-NMR: δ_{H} (400 MHz, CDCl₃, mixture of rotamers) 4.68-4.49 (2H, m), 4.13 (2H, br s), 3.97-3.70 (2H, m), 3.22 (2H, d, J 12.3), 3.04-2.87 (1H, m), 2.78-2.60 (4H, m), 2.06 (2H, m), 1.94 (2H, m), 1.90-1.79 (4H, m), 1.75 (2H, m), 1.60 (2H, m); UPLC (Method A): t_{R} 1.92 min, 98%, MS (ESI) 347.2 (M+H)⁺.

### Example 23: Synthesis of 1-(2-(3,8-diazabicyclo[3.2.1]octan-8-yl)-6,7-dihydrothiazolo[5,4-c]pyridin-5(4H)-yl)-2-phenoxypropan-1-one

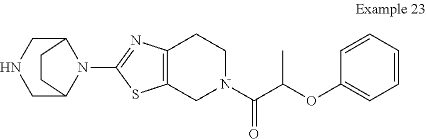 [SMILES: CC(Oc1ccccc1)C(=O)N1CCc2nc(N3C4CCC3CNC4)sc2C1]

Prepared using 2-phenoxypropanoic acid as the acid component.

¹H-NMR: δ_{H} (400 MHz, CDCl₃, mixture of rotamers) 7.31-7.23 (2H, m), 7.04-6.83 (3H, m), 5.11-4.92 (1H, m), 4.89-4.47 (2H, m), 4.11 (2H, br s), 4.02 (1H, m), 3.90-3.61 (1H, m), 3.28-3.12 (2H, m), 2.80-2.48 (4H, m), 2.14-1.86 (4H, m), 1.70-1.60 (3H, m); UPLC (Method A): t_{R} 1.53 min, 99%, MS (ESI) 399.6 (M+H)⁺.

### Example 24: Synthesis of 1-(2-(3,8-diazabicyclo[3.2.1]octan-8-yl)-6,7-dihydrothiazolo[5,4-c]pyridin-5(4H)-yl)-2-cyclopentyl-2,2-difluoroethan-1-one

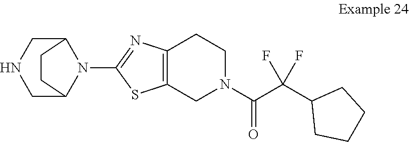 [SMILES: O=C(N1CCc2nc(N3C4CCC3CNC4)sc2C1)C(F)(F)C1CCCC1]

Prepared using 2-cyclopentyl-2,2-difluoroacetic acid as the acid component.

¹H-NMR: δ_{H} (400 MHz, CDCl₃, mixture of rotamers) 4.77-4.58 (2H, m), 4.17-4.08 (2H, m), 4.03-3.86 (2H, m), 3.27-3.14 (2H, m), 2.76 (3H, m), 2.71-2.63 (2H, m), 2.13-1.99 (2H, m), 1.99-1.90 (2H, m), 1.88 (2H, s), 1.59 (6H s); UPLC (Method A): t_{R} 2.91 min, 100%, MS (ESI) 397.2 (M+H)⁺.

### Example 25: Synthesis of 1-(2-(3,8-diazabicyclo[3.2.1]octan-8-yl)-6,7-dihydrothiazolo[5,4-c]pyridin-5(4H)-yl)-2-(4-fluorophenyl)ethan-1-one

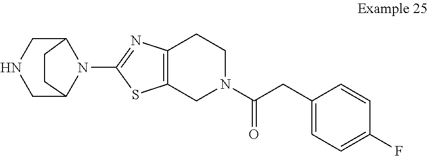 [SMILES: O=C(Cc1ccc(F)cc1)N1CCc2nc(N3C4CCC3CNC4)sc2C1]

Prepared using 2-(4-fluorophenyl)acetic acid as the acid component.

¹H-NMR: δ_{H} (400 MHz, CDCl₃, mixture of rotamers) 7.27-7.14 (2H, m), 7.01 (2H, m), 4.79-4.40 (2H, m), 4.11 (2H, s), 3.98-3.66 (4H, m), 3.21 (2H, d, J 12.2), 2.81-2.45 (4H, m), 2.18-1.84 (4H, m); UPLC (Method C): t_{R} 3.84 min, 98%, MS (ESI) 387.2 (M+H)⁺.

### Example 26: Synthesis of 1-(2-(3,8-diazabicyclo[3.2.1]octan-8-yl)-6,7-dihydrothiazolo[5,4-c]pyridin-5(4H)-yl)-3,3,3-trifluoropropan-1-one

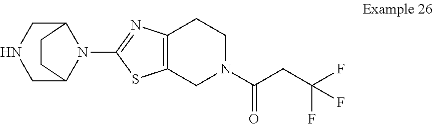 [SMILES: O=C(CC(F)(F)F)N1CCc2nc(N3C4CCC3CNC4)sc2C1]

Prepared using 3,3,3-trifluoropropanoic acid as the acid component.

¹H-NMR: δ_{H} (400 MHz, CDCl₃, mixture of rotamers) 4.71-4.47 (2H, m), 4.13 (2H, s), 3.99-3.72 (2H, m), 3.41-3.28 (2H, m), 3.27-3.18 (2H, m), 2.82-2.63 (4H, m), 2.14-1.88 (4H, m); UPLC (Method A): t_{R} 1.12 min, 91%, MS (ESI) 361.2 (M+H)⁺.

### Example 27: Synthesis of 1-(2-(3,8-diazabicyclo[3.2.1]octan-8-yl)-6,7-dihydrothiazolo[5,4-c]pyridin-5(4H)-yl)-2-(1-methylcyclopentyl)ethan-1-one

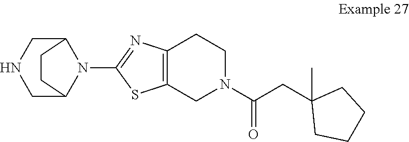 [SMILES: CC1(CC(=O)N2CCc3nc(N4C5CCC4CNC5)sc3C2)CCCC1]

Prepared using 2-(1-methylcyclopentyl)acetic acid as the acid component.

¹H-NMR: δ_{H} (400 MHz, CDCl₃, mixture of rotamers) 4.65-4.46 (2H, m), 4.13 (2H, s), 3.95-3.71 (2H, m), 3.22 (2H, d, J 12.2), 2.77-2.61 (4H, m), 2.49-2.37 (2H, m), 2.12-2.01 (2H, m), 1.99-1.89 (2H, m), 1.71-1.57 (6H, m), 1.53-1.43 (2H, m), 1.12-1.03 (3H, m); UPLC (Method A): t_{R} 1.67 min, 100%, MS (ESI) 375.2 (M+H)⁺.

### Example 28: Synthesis of 1-(2-(3,8-diazabicyclo[3.2.1]octan-8-yl)-6,7-dihydrothiazolo[5,4-c]pyridin-5(4H)-yl)-2-hydroxy-3-methylbutan-1-one

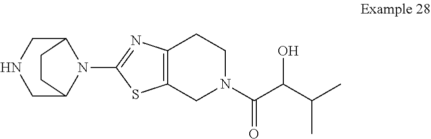 [SMILES: CC(C)C(O)C(=O)N1CCc2nc(N3C4CCC3CNC4)sc2C1]

Prepared using 2-hydroxy-3-methylbutanoic acid as the acid component.

¹H-NMR: δ_{H} (400 MHz, CDCl₃, mixture of rotamers) 4.69-4.40 (2H, m), 4.37-4.25 (1H, m), 4.17-4.10 (2H, m), 4.09-3.79 (1H, m), 3.75-3.56 (2H, m), 3.22 (2H, d, J 12.2), 2.79-2.71 (2H, m), 2.67 (2H, d, J 12.5), 2.12-2.00 (2H, m), 1.97-1.84 (2H, m), 1.14-1.05 (3H, m), 0.83-0.79 (3H, m); UPLC (Method A): t_{R} 1.58 min, 97%, MS (ESI) 351.2 (M+H)⁺.

### Example 29: Synthesis of (2-(3,8-diazabicyclo[3.2.1]octan-8-yl)-6,7-dihydrothiazolo[5,4-c]pyridin-5(4H)-yl)(3,3-difluorocyclopentyl)methanone

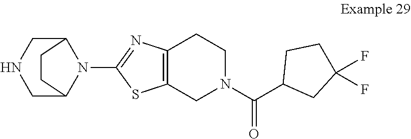 [SMILES: O=C(C1CCC(F)(F)C1)N1CCc2nc(N3C4CCC3CNC4)sc2C1]

Prepared using 3,3-difluorocyclopentanecarboxylic acid as the acid component.

¹H-NMR: δ_{H} (400 MHz, CDCl₃, mixture of rotamers) 4.67-4.61 (1H, m), 4.55-4.48 (0.5H, m), 4.14 (2H, br s), 3.99-3.84 (1H, m), 3.77 (1H, t, J 5.8), 3.31-3.14 (3.5H, m), 2.78-2.45 (5H, m), 2.27 (2H, m), 2.17-1.93 (7H, m); UPLC (Method A): t_{R} 1.37 min, 99%, MS (ESI) 383.2 (M+H)⁺.

### Example 30: Synthesis of 1-(2-(3,8-diazabicyclo[3.2.1]octan-8-yl)-6,7-dihydrothiazolo[5,4-c]pyridin-5(4H)-yl)-2-methyl-2-phenoxypropan-1-one

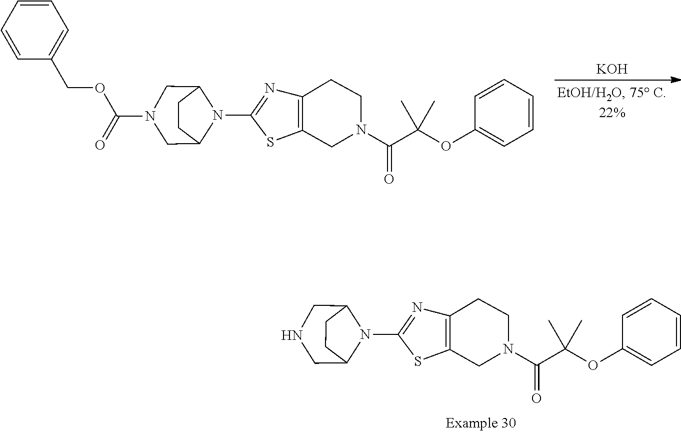 [SMILES: CC(C)(Oc1ccccc1)C(=O)N1CCc2nc(N3C4CCC3CN(C(=O)OCc3ccccc3)C4)sc2C1 ; "KOH" ; CC(C)(Oc1ccccc1)C(=O)N1CCc2nc(N3C4CCC3CNC4)sc2C1]

Benzyl 8-(5-(2-methyl-2-phenoxypropanoyl)-4,5,6,7-tetrahydrothiazolo[5,4-c]pyridin-2-yl)-3,8-diazabicyclo[3.2.1]octane-3-carboxylate was prepared according to the method of Example 1.

To a solution of benzyl 8-(5-(2-methyl-2-phenoxypropanoyl)-4,5,6,7-tetrahydrothiazolo[5,4-c]pyridin-2-yl)-3,8-diazabicyclo[3.2.1]octane-3-carboxylate (87 mg, 0.16 mmol) in ethanol (0.7 mL) was added a solution of potassium hydroxide (93 mg, 1.66 mmol) in water (0.5 mL) and the mixture was stirred at 75° C. for 16 hours. The mixture was diluted with water (10 mL) and extracted with dichloromethane twice. The combined organic layers were washed with brine and concentrated in vacuo. The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-methyl-2-phenoxypropan-1-one (16 mg, 22% yield, Example 30) as a light yellow solid. ¹H-NMR: δ_{H} (400 MHz, CDCl₃) 7.19 (2H, m), 6.93 (1H, m), 6.83 (2H, d, J 8.0), 4.92-4.58 (2H, m), 4.18-3.84 (4H, m), 3.18 (2H, t, J 11.1), 2.74-2.58 (3H, m), 2.51-2.40 (1H, m), 2.02 (2H, m), 1.91 (2H, m), 1.63 (3H, s), 1.59 (3H, s); UPLC (Method A): t_{R} 1.68 min, 94%, MS (ESI) 413.2 (M+H)⁺.

### Example 31: Synthesis of (2-(3,8-diazabicyclo[3.2.1]octan-8-yl)-6,7-dihydrothiazolo[5,4-c]pyridin-5(4H)-yl)(1-phenoxycyclopropyl)methanone

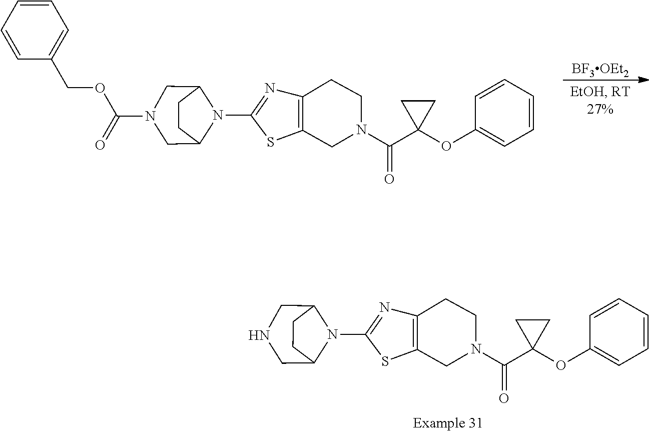 [SMILES: O=C(OCc1ccccc1)N1CC2CCC(C1)N2c1nc2c(s1)CN(C(=O)C1(Oc3ccccc3)CC1)CC2 ; O=C(N1CCc2nc(N3C4CCC3CNC4)sc2C1)C1(Oc2ccccc2)CC1 ; "BF3·OEt2"]

Benzyl 8-(5-(1-phenoxycyclopropane-1-carbonyl)-4,5,6,7-tetrahydrothiazolo[5,4-c]pyridin-2-yl)-3,8-diazabicyclo[3.2.1]octane-3-carboxylate was prepared according to Example 1.

A solution of benzyl 8-(5-(1-phenoxycyclopropane-1-carbonyl)-4,5,6,7-tetrahydrothiazolo[5,4-c]pyridin-2-yl)-3,8-diazabicyclo[3.2.1]octane-3-carboxylate (76 mg, 0.13 mmol) in dichloromethane (3 mL) was added to a mixture of boron trifluoride etherate (ca. 48% BF₃, 1 mL, 7.9 mmol) and trifluoroacetic acid (1 mL, 13.1 mmol) in ethanol (0.6 mL) and stirred at room temperature for 2 hours. The mixture was slowly poured into saturated aqueous sodium carbonate solution and stirred for 15 minutes while effervescence was observed. The mixture was diluted with ethyl acetate and the layers were separated. The aqueous layer was extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford (2-(3,8-diazabicyclo[3.2.1]octan-8-yl)-6,7-dihydrothiazolo[5,4-c]pyridin-5(4H)-yl)(1-phenoxycyclopropyl)methanone (16 mg, 27%, Example 31) as a white solid. ¹H-NMR: δ_{H} (400 MHz, CDCl₃, mixture of rotamers) 7.26-7.20 (2H, m), 7.04 (2H, d, J 8.1), 7.00-6.90 (1H, m), 4.89-4.44 (2H, m), 4.21-3.70 (4H, m), 3.29-3.12 (2H, m), 2.74-2.56 (4H, m), 2.12-1.85 (4H, m), 1.52-1.44 (2H, m), 1.21-1.09 (2H, m); UPLC (Method A): t_{R} 1.69 min, 100%, MS (ESI) 411.2 (M+H)⁺.

The following examples (32-38) were prepared using procedures analogous to Example 1 and Example 31, using the appropriate starting materials.

### Example 32: Synthesis of 1-(2-(3,8-diazabicyclo[3.2.1]octan-8-yl)-6,7-dihydrothiazolo[5,4-c]pyridin-5(4H)-yl)-3,3,3-trifluoro-2,2-dimethylpropan-1-one

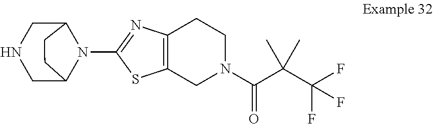 [SMILES: CC(C)(C(=O)N1CCc2nc(N3C4CCC3CNC4)sc2C1)C(F)(F)F]

Prepared using 3,3,3-trifluoro-2,2-dimethylpropanoic acid as the acid component.

¹H-NMR: δ_{H} (400 MHz, CDCl₃, mixture of rotamers) 4.68-4.58 (2H, m), 4.13 (2H, s), 3.90 (2H, t, J5.7), 3.22 (2H, m), 2.79-2.71 (2H, m), 2.71-2.62 (2H, m), 2.13-2.00 (2H, m), 1.99-1.89 (2H, m), 1.57 (6H, s); UPLC (Method A): t_{R} 1.46 min, 97%, MS (ESI) 389.2 (M+H)⁺.

### Example 33: Synthesis of (2-(3,8-diazabicyclo[3.2.1]octan-8-yl)-6,7-dihydrothiazolo[5,4-c]pyridin-5(4H)-yl)(1-phenylcyclopropyl)methanone

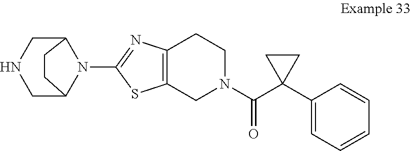 [SMILES: O=C(N1CCc2nc(N3C4CCC3CNC4)sc2C1)C1(c2ccccc2)CC1]

Prepared using 1-phenylcyclopropane-1-carboxylic acid as the acid component.

¹H-NMR: δ_{H} (400 MHz, CDCl₃, mixture of rotamers) 7.33-7.27 (2H, m), 7.25-7.11 (3H, m), 4.70-4.32 (2H, m), 4.09 (2H, s), 3.96-3.66 (2H, m), 3.19 (2H, dd, J 12.6, 1.7), 2.64 (2.5H, m), 2.30-1.88 (5.5H, m), 1.46 (2H, s), 1.22 (2H, s); UPLC (Method A): t_{R} 1.53 min, 93%, MS (ESI) 395.2 (M+H)⁺.

### Example 34: Synthesis of 1-(2-(3,8-diazabicyclo[3.2.1]octan-8-yl)-6,7-dihydrothiazolo[5,4-c]pyridin-5(4H)-yl)-2-cyclopentyl-2-fluoroethan-1-one

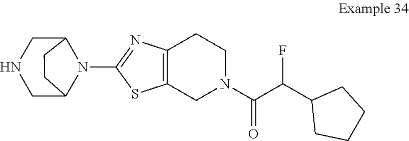 [SMILES: O=C(C(F)C1CCCC1)N1CCc2nc(N3C4CCC3CNC4)sc2C1]

Prepared using 2-cyclopentyl-2-fluoroacetic acid as the acid component.

¹H-NMR: δ_{H} (400 MHz, CDCl₃, mixture of rotamers) 5.10-4.80 (1H, m), 4.74-4.50 (2H, m), 4.13 (2H, t, J 3.6), 4.01-3.71 (2H, m), 3.22 (2H, d, J 12.2), 2.84-2.59 (4H, m), 2.58-2.31 (1H, m), 2.18-1.66 (8H, m), 1.38 (1H, m); UPLC (Method A): t_{R} 2.27 min, 99%, MS (ESI) 379.2 (M+H)⁺.

### Example 35: Synthesis of 1-(2-(3,8-diazabicyclo[3.2.1]octan-8-yl)-6,7-dihydrothiazolo[5,4-c]pyridin-5(4H)-yl)-2-(4,4-difluorocyclohexyl)-2-fluoroethan-1-one

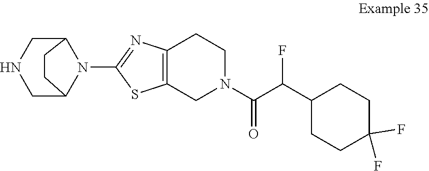 [SMILES: O=C(C(F)C1CCC(F)(F)CC1)N1CCc2nc(N3C4CCC3CNC4)sc2C1]

Prepared using 2-(4,4-difluorocyclohexyl)-2-fluoroacetic acid as the acid component.

¹H-NMR: δ_{H} (400 MHz, CDCl₃, mixture of rotamers) 5.04-4.80 (1H, m), 4.72-4.52 (2H, m), 4.18-4.04 (2H, m), 4.02-3.73 (2H, m), 3.22 (2H, d, J 12.5), 2.81-2.59 (4H, m), 2.24-1.98 (6H, m), 1.98-1.66 (6H, m), 1.58-1.41 (2H, m); UPLC (Method A): t_{R} 2.26 min, 99%, MS (ESI) 429.2 (M+H)⁺.

### Example 36: Synthesis of 1-(2-(3,8-diazabicyclo[3.2.1]octan-8-yl)-6,7-dihydrothiazolo[5,4-c]pyridin-5(4H)-yl)-2,2-difluoro-2-phenylethan-1-one

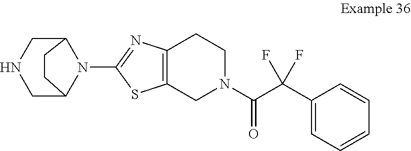 [SMILES: O=C(N1CCc2nc(N3C4CCC3CNC4)sc2C1)C(F)(F)c1ccccc1]

Prepared using 2,2-difluoro-2-phenylacetic acid as the acid component.

¹H-NMR: δ_{H} (400 MHz, CDCl₃, mixture of rotamers) 7.67-7.37 (5H, m), 4.76-4.44 (2H, m), 4.11 (2H, s), 4.03-3.67 (2H, m), 3.29-3.13 (2H, m), 2.83-2.40 (4H, m), 2.12-1.89 (4H, m); UPLC (Method C): t_{R} 4.18 min, 96%, MS (ESI) 405.2 (M+H)⁺.

### Example 37: Synthesis of (2-(3,8-diazabicyclo[3.2.1]octan-8-yl)-6,7-dihydrothiazolo[5,4-c]pyridin-5(4H)-yl)(2,2-dimethylcyclopentyl)methanone

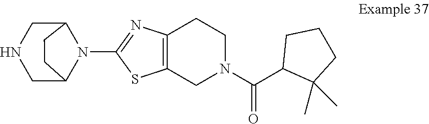 [SMILES: CC1(C)CCCC1C(=O)N1CCc2nc(N3C4CCC3CNC4)sc2C1]

Prepared using 2,2-dimethylcyclopentane-1-carboxylic acid as the acid component.

¹H-NMR: δ_{H} (400 MHz, CDCl₃, mixture of rotamers) 4.90-4.80 (1H, m), 4.68-4.47 (1H, m), 4.46-4.38 (1H, m), 4.22-4.09 (2H, m), 4.00-3.88 (1H, m), 3.75-3.61 (1H, m), 3.22 (2H, m), 2.88-2.62 (5H, m), 2.29-2.11 (1H, m), 2.12-1.98 (2H, m), 1.97-1.89 (2H, m), 1.89-1.76 (2H, m), 1.73-1.62 (2H, m), 1.50-1.38 (1H, m), 1.17-1.05 (3H, m), 1.00-0.85 (3H, m); UPLC (Method A): t_{R} 1.64 min, 100%, MS (ESI) 375.2 (M+H)⁺.

### Example 38: Synthesis of (2-(3,8-diazabicyclo[3.2.1]octan-8-yl)-6,7-dihydrothiazolo[5,4-c]pyridin-5(4H)-yl)(3,3-dimethylcyclopentyl)methanone

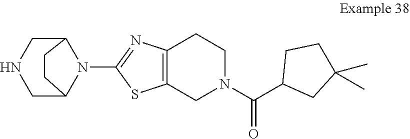 [SMILES: CC1(C)CCC(C(=O)N2CCc3nc(N4C5CCC4CNC5)sc3C2)C1]

Prepared using 3,3-dimethylcyclopentane-1-carboxylic acid as the acid component.

¹H-NMR: δ_{H} (400 MHz, CDCl₃, mixture of rotamers) 4.65-4.48 (2H, m), 4.13 (2H, s), 3.93-3.72 (2H, m), 3.22 (2H, m), 3.18-3.05 (1H, m), 2.77-2.60 (4H, m), 2.12-1.84 (6H, m), 1.80-1.60 (3H, m), 1.48-1.37 (1H, m), 1.11-1.05 (3H, m), 1.04-0.96 (3H, m); UPLC (Method A): t_{R} 1.64 min, 100%, MS (ESI) 375.2 (M+H)⁺.

### Example 50: Synthesis of 2-(3,8-diazabicyclo[3.2.1]octan-8-yl)-N-cyclopentyl-6,7-dihydrothiazolo[5,4-c]pyridine-5(4H)-carboxamide

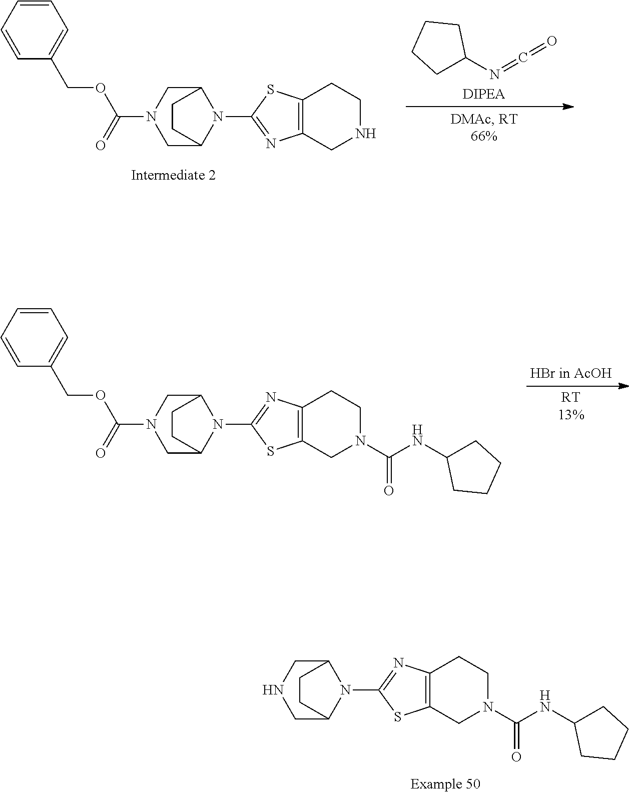 [SMILES: O=C(OCc1ccccc1)N1CC2CCC(C1)N2c1nc2c(s1)CCNC2 ; O=C=NC1CCCC1 ; O=C(NC1CCCC1)N1CCc2nc(N3C4CCC3CN(C(=O)OCc3ccccc3)C4)sc2C1 ; O=C(NC1CCCC1)N1CCc2nc(N3C4CCC3CNC4)sc2C1]

To a solution of benzyl 8-(4,5,6,7-tetrahydrothiazolo[5,4-c]pyridin-2-yl)-3,8-diazabicyclo[3.2.1]octane-3-carboxylate (150 mg, 0.39 mmol, Intermediate 2) and N,N-diisopropylethylamine (0.14 mL, 0.78 mmol) in N,N-dimethylacetamide (3 mL) was added isocyanatocyclopentane (0.05 mL, 0.47 mmol) and the mixture was stirred at room temperature for 16 hours. The crude mixture was purified with preparative reversed phase chromatography (Phenomenex LUNA C18 column, formic acid in water/acetonitrile) and lyophilized to afford benzyl 8-(5-(cyclopentylcarbamoyl)-4,5,6,7-tetrahydrothiazolo[5,4-c]pyridin-2-yl)-3,8-diazabicyclo[3.2.1]octane-3-carboxylate (135 mg, 66%) as a white solid. A solution of benzyl 8-(5-(cyclopentylcarbamoyl)-4,5,6,7-tetrahydrothiazolo[5,4-c]pyridin-2-yl)-3,8-diazabicyclo[3.2.1]octane-3-carboxylate (135 mg, 0.27 mmol) in 33% hydrogen bromide in acetic acid (5 mL, 87 mmol) was stirred at room temperature for 2 hours. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-N-cyclopentyl-6,7-dihydrothiazolo[5,4-c]pyridine-5(4H)-carboxamide (14 mg, 13%, Example 50) as a white solid. ¹H-NMR: δ_{H} (400 MHz, CDCl₃, mixture of rotamers) 6.35 (1H, d, J7.0), 4.39-4.26 (2H, m), 4.16-3.94 (2H, m), 3.94-3.85 (1H, m), 3.76-3.54 (2H, m), 3.07-2.87 (2H, m), 1.93-1.69 (7H, m), 1.69-1.53 (3H, m), 1.54-1.31 (5H, m); LCMS (Method A): t_{R} 1.28 min, 98.1, MS (ESI) 362.2 (M+H)⁺.

The following examples (51-58) were prepared using procedures analogous to Example 50, using the appropriate starting materials.

### Example 51: Synthesis of 2-(3,8-diazabicyclo[3.2.1]octan-8-yl)-N-isopropyl-6,7-dihydrothiazolo[5,4-c]pyridine-5(4H)-carboxamide

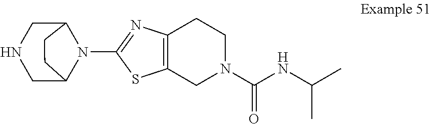 [SMILES: CC(C)NC(=O)N1CCc2nc(N3C4CCC3CNC4)sc2C1]

Prepared using 2-isocyanatopropane as the isocyanate component.

¹H-NMR: δ_{H} (400 MHz, CDCl₃) 4.46-4.39 (2H, m), 4.31-4.13 (3H, m), 4.07-3.96 (1H, m), 3.67-3.61 (2H, m), 3.26 (1H, d, J 12.7), 2.79-2.65 (4H, m), 2.17-2.00 (4H, m), 1.21-1.11 (8H, m); UPLC (Method B): t_{R} 0.92 min, 91%, MS (ESI) 336.1 (M+H)⁺.

### Example 52: Synthesis of 2-(3,8-diazabicyclo[3.2.1]octan-8-yl)-N-isopropyl-6,7-dihydrothiazolo[5,4-c]pyridine-5(4H)-carboxamide

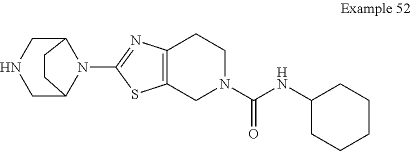 [SMILES: O=C(NC1CCCCC1)N1CCc2nc(N3C4CCC3CNC4)sc2C1]

Prepared using isocyanatocyclohexane as the isocyanate component.

¹H-NMR: δ_{H} (400 MHz, CDCl₃) 4.42 (2H, t, J2.0), 4.32 (1H, d, J7.5), 4.14 (2H, s), 3.77-3.57 (3H, m), 3.29-3.18 (2H, m), 2.74-2.63 (4H, m), 2.11-1.90 (6H, m), 1.77-1.67 (3H, m), 1.45-1.03 (6H, m); UPLC (Method B): t_{R} 1.14 min, 100%, MS (ESI) 376.2 (M+H)⁺.

### Example 53: Synthesis of 2-(3,8-diazabicyclo[3.2.1]octan-8-yl)-N-cycloheptyl-6,7-dihydrothiazolo[5,4-c]pyridine-5(4H)-carboxamide

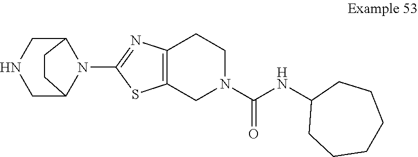 [SMILES: O=C(NC1CCCCCC1)N1CCc2nc(N3C4CCC3CNC4)sc2C1]

Prepared using isocyanatocycloheptane as the isocyanate component.

¹H-NMR: δ_{H} (400 MHz, DMSO-d₆) 6.32 (1H, d, J7.7), 4.33 (2H, s), 3.97 (2H, s), 3.71-3.51 (3H, m), 2.92 (2H, d, J 12.2), 2.49-2.41 (4H, m), 1.96-1.68 (7H, m), 1.66-1.28 (11H, m); UPLC (Method B): t_{R} 1.24 min, 100%, MS (ESI) 390.2 (M+H)⁺.

### Example 54: Synthesis of 2-(3,8-diazabicyclo[3.2.1]octan-8-yl)-N-(cyclohexylmethyl)-6,7-dihydrothiazolo[5,4-c]pyridine-5(4H)-carboxamide

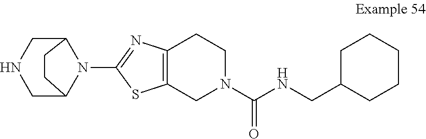 [SMILES: O=C(NCC1CCCCC1)N1CCc2nc(N3C4CCC3CNC4)sc2C1]

Prepared using (isocyanatomethyl)cyclohexane as the isocyanate component.

¹H-NMR: δ_{H} (400 MHz, CDCl₃) 4.66-4.52 (1H, m), 4.44 (2H, s), 4.20-4.06 (2H, m), 3.66 (2H, t, J5.7), 3.30-3.18 (2H, m), 3.10 (2H, t, J 6.2), 2.78-2.62 (4H, m), 2.15-1.89 (4H, m), 1.69-1.59 (6H, m), 1.53-1.41 (1H, m), 1.31-1.07 (3H, m), 0.99-0.85 (2H, m); UPLC (Method B): t_{R} 1.24 min, 100%, MS (ESI) 390.2 (M+H)⁺.

### Example 55: Synthesis of 2-(3,8-diazabicyclo[3.2.1]octan-8-yl)-N-(4-fluorobenzyl)-6,7-dihydrothiazolo[5,4-c]pyridine-5(4H)-carboxamide

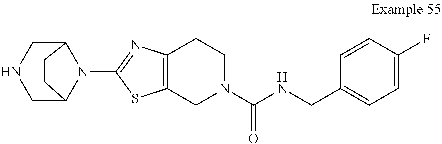 [SMILES: O=C(NCc1ccc(F)cc1)N1CCc2nc(N3C4CCC3CNC4)sc2C1]

Prepared using 1-fluoro-4-(isocyanatomethyl)benzene as the isocyanate component.

¹H-NMR: δ_{H} (400 MHz, DMSO-d₆) 7.33-7.07 (5H, m), 4.39 (2H, d, J2.2), 4.23 (2H, d, J5.7), 4.02-3.93 (2H, m), 3.63 (2H, t, J 5.7), 2.92 (2H, m), 2.56-2.51 (3H, m), 1.99-1.76 (4H, m); UPLC (Method A): t_{R} 0.75 min, 98%, MS (ESI) 402.2 (M+H)⁺.

### Example 56: Synthesis of 2-(3,8-diazabicyclo[3.2.1]octan-8-yl)-N-(4-(trifluoromethyl)benzyl)-6,7-dihydrothiazolo[5,4-c]pyridine-5(4H)-carboxamide

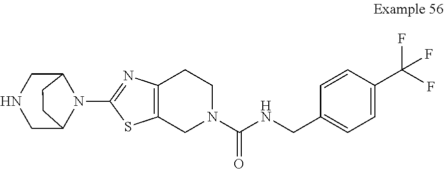 [SMILES: O=C(NCc1ccc(C(F)(F)F)cc1)N1CCc2nc(N3C4CCC3CNC4)sc2C1]

Prepared using 1-(isocyanatomethyl)-4-(trifluoromethyl)benzene as the isocyanate component.

¹H-NMR: δ_{H} (400 MHz, CDCl₃) 7.58 (2H, d, J 8.1), 7.43 (2H, d, J 8.0), 4.89 (1H, s), 4.52 (2H, d, J 5.6), 4.48 (2H, d, J 2.0), 4.13 (2H, d, J 4.2), 3.70 (2H, t, J 5.7), 3.22 (2H, m), 2.76-2.64 (4H, m), 2.10-2.03 (2H, m), 1.99-1.91 (2H, m); UPLC (Method B): t_{R} 1.28 min, 100%, MS (ESI) 452.1 (M+H)⁺.

### Example 57: Synthesis of 2-(3,8-diazabicyclo[3.2.1]octan-8-yl)-N-phenyl-6,7-dihydrothiazolo[5,4-c]pyridine-5(4H)-carboxamide

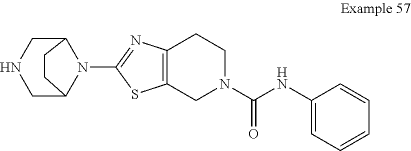 [SMILES: O=C(Nc1ccccc1)N1CCc2nc(N3C4CCC3CNC4)sc2C1]

Prepared using isocyanatobenzene as the isocyanate component.

¹H-NMR: δ_{H} (400 MHz, CDCl₃) 7.39-7.27 (5H, m), 7.09-7.01 (1H, m), 4.57 (2H, s), 4.15 (2H, d, J 4.8), 3.86-3.73 (2H, m), 3.29-3.17 (2H, m), 2.85-2.73 (2H, m), 2.68 (2H, dd, J 12.5, 2.3), 2.14-2.00 (2H, m), 2.00-1.91 (2H, m); UPLC (Method B): t_{R} 0.71 min, 91%, MS (ESI) 370.2 (M+H)⁺.

### Example 58: Synthesis of 2-(3,8-diazabicyclo[3.2.1]octan-8-yl)-N-(4,4-difluorocyclohexyl)-6,7-dihydrothiazolo[5,4-c]pyridine-5(4H)-carboxamide

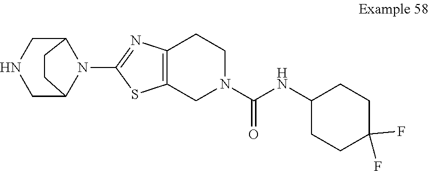 [SMILES: O=C(NC1CCC(F)(F)CC1)N1CCc2nc(N3C4CCC3CNC4)sc2C1]

Prepared using 1,1-difluoro-4-isocyanatocyclohexane as the isocyanate component.

¹H-NMR: δ_{H} (400 MHz, CDCl₃, mixture of rotamers) 4.45-4.39 (2H, m), 4.32 (1H, d, J 7.5), 4.14 (2H, s), 3.90-3.76 (1H, m), 3.67 (2H, m), 3.28-3.17 (2H, m), 2.75-2.64 (4H, m), 2.02-1.74 (9H, m), 1.57-1.44 (3H, m); UPLC (Method A): t_{R} 1.39 min, 93%, MS (ESI) 412.2 (M+H)⁺.

### Example 60: Synthesis of (2-(3,8-diazabicyclo[3.2.1]octan-8-yl)-6,7-dihydrothiazolo[5,4-c]pyridin-5(4H)-yl)(pyrrolidin-1-yl)methanone

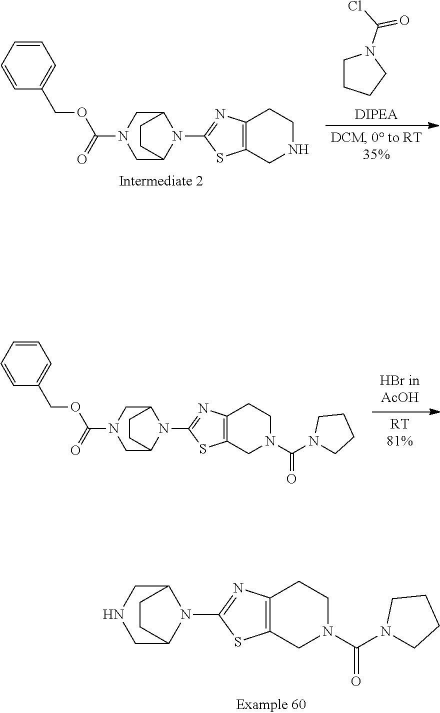 [SMILES: O=C(OCc1ccccc1)N1CC2CCC(C1)N2c1nc2c(s1)CNCC2 ; O=C(Cl)N1CCCC1 ; O=C(OCc1ccccc1)N1CC2CCC(C1)N2c1nc2c(s1)CN(C(=O)N1CCCC1)CC2 ; O=C(N1CCCC1)N1CCc2nc(N3C4CCC3CNC4)sc2C1]

A solution of benzyl 8-(4,5,6,7-tetrahydrothiazolo[5,4-c]pyridin-2-yl)-3,8-diazabicyclo[3.2.1]octane-3-carboxylate (67 mg, 0.17 mmol, Intermediate 2) and N,N-diisopropylethylamine (91 μL, 0.52 mmol) in dichloromethane (2.5 mL) was cooled to 0° C. and 1-pyrrolidinecarbonyl chloride (35 μL, 0.32 mmol) was added. The mixture was allowed to warm to room temperature and stirred for 16 hours. The mixture was purified with preparative reversed phase chromatography (Waters XSelect™ CSH C18 column, ammonium bicarbonate in water/acetonitrile) and lyophilized to afford benzyl 8-(5-(pyrrolidine-1-carbonyl)-4,5,6,7-tetrahydrothiazolo[5,4-c]pyridin-2-yl)-3,8-diazabicyclo[3.2.1]octane-3-carboxylate (29 mg, 35%) as a yellow solid.

A solution of benzyl 8-(5-(pyrrolidine-1-carbonyl)-4,5,6,7-tetrahydrothiazolo[5,4-c]pyridin-2-yl)-3,8-diazabicyclo[3.2.1]octane-3-carboxylate (29 mg, 0.06 mmol) in 33% hydrogen bromide in acetic acid (0.16 mL, 0.91 mmol) was stirred at room temperature for 16 hours. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford (2-(3,8-diazabicyclo[3.2.1]octan-8-yl)-6,7-dihydrothiazolo[5,4-c]pyridin-5(4H)-yl)(pyrrolidin-1-yl)methanone (17 mg, 81%, Example 60) as a white solid. ¹H-NMR: δ_{H} (400 MHz, CDCl₃) 4.30 (2H, t, J 2.0), 4.16-4.10 (2H, m), 3.55 (2H, t, J5.7), 3.45-3.37 (4H, m), 3.23 (2H, m), 2.77-2.70 (2H, m), 2.66 (2H, m), 2.08-2.02 (2H, m), 1.96-1.89 (2H, m), 1.88-1.81 (4H, m); UPLC (Method A): t_{R} 1.24 min, 97%, MS (ESI) 348.2 (M+H)⁺.

### Example 62: Synthesis of 2-(3,8-diazabicyclo[3.2.1]octan-8-yl)-N-cyclopentyl-N-methyl-6,7-dihydrothiazolo[5,4-c]pyridine-5(4H)-carboxamide

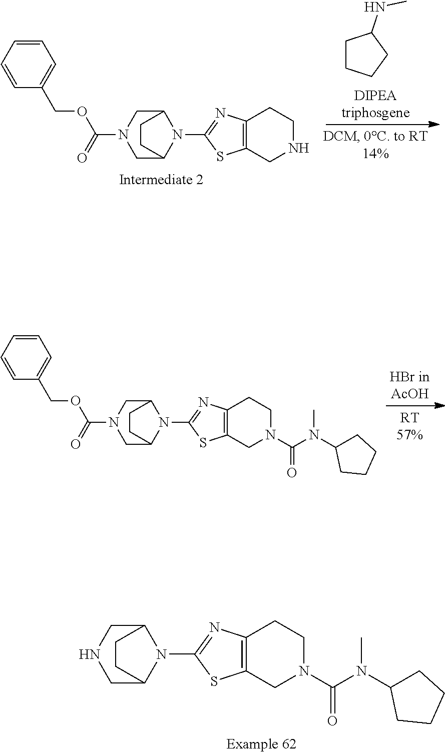

To a solution of benzyl 8-(4,5,6,7-tetrahydrothiazolo[5,4-c]pyridin-2-yl)-3,8-diazabicyclo[3.2.1]octane-3-carboxylate (77 mg, 0.20 mmol, Intermediate 2) in dichloromethane (3 mL) at 0° C. were added N,N-diisopropylethylamine (88 μL, 0.51 mmol) and triphosgene (42 mg, 0.14 mmol). The mixture was stirred at 0° C. for 30 minutes. Next, N-methylcyclopentanamine (118 μL, 1.00 mmol) was added and the mixture was stirred at room temperature for 1.5 hours. The mixture was purified with silica column chromatography (50% to 100% ethyl acetate in n-heptane followed by 0% to 10% methanol in dichloromethane) and concentrated in vacuo to afford a brown oil. The oil was further purified with preparative reversed phase chromatography (Waters XSelect™ CSH C18 column, ammonium bicarbonate in water/acetonitrile) and lyophilized to afford benzyl 8-(5-(cyclopentyl(methyl)carbamoyl)-4,5,6,7-tetrahydrothiazolo[5,4-c]pyridin-2-yl)-3,8-diazabicyclo[3.2.1]octane-3-carboxylate (14 mg, 14%) as a yellow solid.

To a solution of benzyl 8-(5-(cyclopentyl(methyl)carbamoyl)-4,5,6,7-tetrahydrothiazolo[5,4-c]pyridin-2-yl)-3,8-diazabicyclo[3.2.1]octane-3-carboxylate (14 mg, 0.03 mmol) in acetic acid (1 mL) was added 33% hydrogen bromide in acetic acid (49 μL, 0.28 mmol) and the mixture was stirred at room temperature for 25 hours. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-N-cyclopentyl-N-methyl-6,7-dihydrothiazolo[5,4-c]pyridine-5(4H)-carboxamide (6 mg, 57%, Example 62) as a white solid. ¹H-NMR: δ_{H} (400 MHz, CDCl₃) 4.25 (2H, t, J 2.0), 4.20-4.10 (3H, m), 3.50 (2H, t, J 5.6), 3.23 (2H, d, J 12.1), 2.76 (3H, s), 2.75-2.70 (2H, m), 2.69-2.60 (2H, m), 2.08-2.02 (2H, m), 1.96-1.90 (2H, m), 1.88-1.78 (2H, m), 1.71-1.64 (2H, m), one signal (4H) coincides with H₂O signal; UPLC (Method A): t_{R} 2.22 min, 96%, MS (ESI) 376.4 (M+H)⁺.

### Example 65: Synthesis of 1-(2-(3,8-diazabicyclo[3.2.1]octan-8-yl)-6,7-dihydrothiazolo[5,4-c]pyridin-5(4H)-yl)-2,2-dimethylpropan-1-one

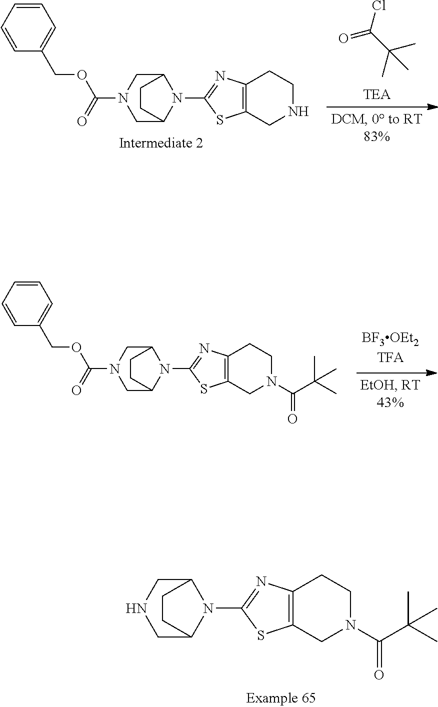

A solution of benzyl 8-(4,5,6,7-tetrahydrothiazolo[5,4-c]pyridin-2-yl)-3,8-diazabicyclo[3.2.1]octane-3-carboxylate (250 mg, 0.650 mmol, Intermediate 2) in dichloromethane (2 mL) was cooled to 0° C. Triethylamine (0.19 mL, 1.37 mmol) and pivaloyl chloride (0.10 mL, 0.78 mmol) were added, the mixture was allowed to reach room temperature and was stirred for 16 hours. The mixture was quenched with ice water and diluted with some dichloromethane. The layers were separated, and the aqueous layer was extracted with dichloromethane twice. The combined organics were washed with brine, dried with sodium sulfate and concentrated in vacuo. The residue was purified with silica column chromatography (35% to 65% ethyl acetate in n-heptane) and concentrated in vacuo to afford benzyl 8-(5-pivaloyl-4,5,6,7-tetrahydrothiazolo[5,4-c]pyridin-2-yl)-3,8-diazabicyclo[3.2.1]octane-3-carboxylate (253 mg, 83%) as a white solid.

A solution of benzyl 8-(5-pivaloyl-4,5,6,7-tetrahydrothiazolo[5,4-c]pyridin-2-yl)-3,8-diazabicyclo[3.2.1]octane-3-carboxylate (133 mg, 0.28 mmol) in dichloromethane (3 mL) was added to a mixture of boron trifluoride etherate ca. 48% BF3 (2.7 mL, 21.3 mmol) and trifluoroacetic acid (2.7 mL, 35.3 mmol) in ethanol (0.6 mL) and was stirred at room temperature for 2 hours. The mixture was slowly poured into saturated aqueous sodium carbonate solution and stirred for 15 minutes while effervescence was observed. The mixture was diluted with ethyl acetate and the layers were separated. The aqueous layer was extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2,2-dimethylpropan-1-one (41 mg, 43%, Example 65) as a white solid. ¹H-NMR: δ_{H} (400 MHz, CDCl₃) 4.72-4.48 (2H, m), 4.21-4.03 (2H, m), 3.89 (2H, t, J5.7), 3.23 (2H, d, J 12.5), 2.83-2.58 (4H, m), 2.15-1.85 (4H, m), 1.32 (9H, s); UPLC (Method A): t_{R} 1.76 min, 99%, MS (ESI) 335.2 (M+H)⁺.

### Example 66: Synthesis of 1-(2-(3,8-diazabicyclo[3.2.1]octan-8-yl)-4,6-dihydro-5H-pyrrolo[3,4-d]thiazol-5-yl)-2-cyclopentylethan-1-one

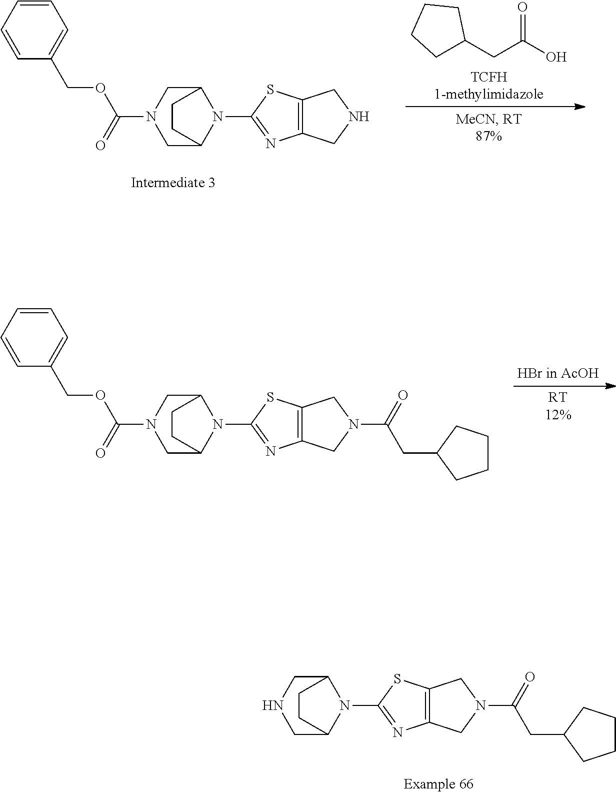 [SMILES: O=C(OCc1ccccc1)N1CC2CCC(C1)N2c1nc2c(s1)CNC2 ; O=C(O)CC1CCCC1 ; O=C(CC1CCCC1)N1Cc2nc(N3C4CCC3CN(C(=O)OCc3ccccc3)C4)sc2C1 ; O=C(CC1CCCC1)N1Cc2nc(N3C4CCC3CNC4)sc2C1]

To a solution of 1-methylimidazole (0.02 mL, 0.26 mmol) and cyclopentylacetic acid (0.03 mL, 0.23 mmol) in acetonitrile (0.5 mL) was added chloro-N,N,N′,N′-tetramethylformamidinium hexafluorophosphate (64 mg, 0.23 mmol) and the mixture was stirred at room temperature for 15 minutes. This mixture was added to a solution of benzyl 8-(5,6-dihydro-4H-pyrrolo[3,4-d]thiazol-2-yl)-3,8-diazabicyclo[3.2.1]octane-3-carboxylate (65 mg, 0.18 mmol, Intermediate 3) in acetonitrile (0.5 mL) and was stirred at room temperature for 1 hour. The mixture was poured into water and extracted with diethyl ether twice. The combined organic layers were washed with brine, dried with sodium sulfate and concentrated in vacuo to afford benzyl 8-(5-(2-cyclopentylacetyl)-5,6-dihydro-4H-pyrrolo[3,4-d]thiazol-2-yl)-3,8-diazabicyclo[3.2.1]octane-3-carboxylate (73 mg, 87%) as an orange oil.

A solution of benzyl 8-(5-(2-cyclopentylacetyl)-5,6-dihydro-4H-pyrrolo[3,4-d]thiazol-2-yl)-3,8-diazabicyclo[3.2.1]octane-3-carboxylate (72 mg, 0.15 mmol) in 33% hydrogen bromide in acetic acid (0.2 mL, 3.46 mmol) was stirred at room temperature for 30 minutes. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-4,6-dihydro-5H-pyrrolo[3,4-d]thiazol-5-yl)-2-cyclopentylethan-1-one (6.3 mg, 12%, Example 66) as a white solid. ¹H-NMR: δ_{H} (400 MHz, CDCl₃, mixture of rotamers) 4.73-4.60 (2H, m), 4.58-4.48 (2H, m), 4.22-4.09 (2H, m), 3.28-3.18 (2H, m), 2.73-2.63 (2H, m), 2.43-2.27 (3H, m), 2.15-2.03 (2H, m), 2.01-1.84 (4H, m), 1.70-1.50 (4H, m), 1.27-1.12 (2H, m); UPLC (Method C): t_{R} 1.28 min, 98%, MS (ESI) 347.2 (M+H)⁺.

The following example (67) was prepared using procedures analogous to Example 66, using the appropriate starting materials.

### Example 67: Synthesis of 1-(2-(3,8-diazabicyclo[3.2.1]octan-8-yl)-4,6-dihydro-5H-pyrrolo[3,4-d]thiazol-5-yl)-2-(4,4-difluorocyclohexyl)ethan-1-one

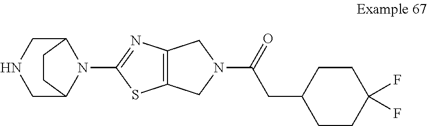 [SMILES: O=C(CC1CCC(F)(F)CC1)N1Cc2nc(N3C4CCC3CNC4)sc2C1]

Prepared using 2-(4,4-difluorocyclohexyl)acetic acid as the acid component.

¹H-NMR: δ_{H} (400 MHz, CDCl₃, mixture of rotamers) 4.71-4.62 (2H, m), 4.56-4.48 (2H, m), 4.20-4.10 (2H, m), 3.29-3.18 (2H, m), 2.74-2.62 (2H, m), 2.30-2.20 (2H, m), 2.15-2.00 (5H, m), 2.00-1.94 (2h, m), 1.94-1.67 (4H, m), 1.45-1.25 (2H, m); UPLC (Method C): t_{R} 4.24 min, 97%, MS (ESI) 397.2 (M+H)⁺.

### Example 68: Synthesis of 2-(3,8-diazabicyclo[3.2.1]octan-8-yl)-N-cyclopentyl-4,6-dihydro-5H-pyrrolo[3,4-d]thiazole-5-carboxamide

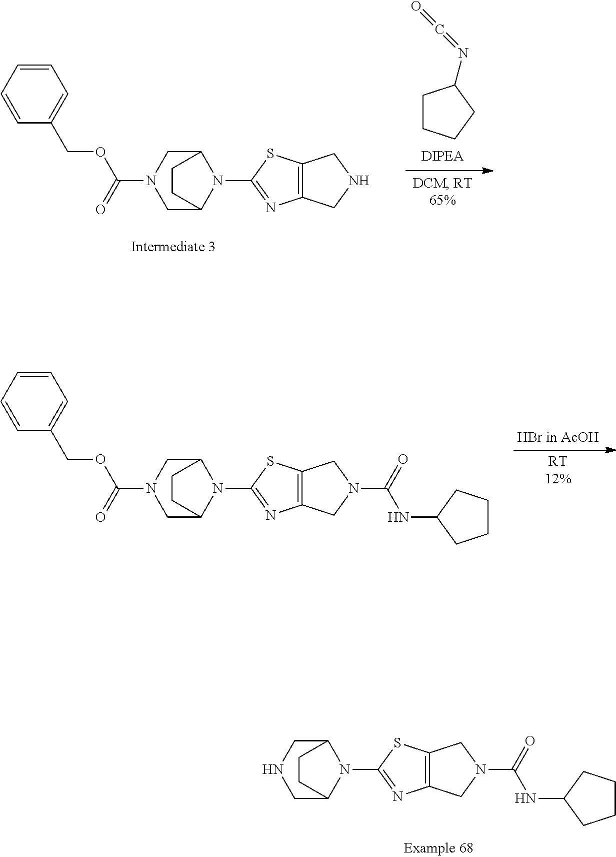 [SMILES: O=C(OCc1ccccc1)N1CC2CCC(C1)N2c1nc2c(s1)CNC2 ; O=C=NC1CCCC1 ; O=C(NC1CCCC1)N1Cc2nc(N3C4CCC3CN(C(=O)OCc3ccccc3)C4)sc2C1 ; O=C(NC1CCCC1)N1Cc2nc(N3C4CCC3CNC4)sc2C1]

To a solution of benzyl 8-(5,6-dihydro-4H-pyrrolo[3,4-d]thiazol-2-yl)-3,8-diazabicyclo[3.2.1]octane-3-carboxylate (65 mg, 0.19 mmol, Intermediate 3) and N,N-diisopropylethylamine (0.06 mL, 0.35 mmol in dichloromethane (1 mL) was added cyclopentyl isocyanate (0.03 mL, 0.23 mmol) and the mixture was stirred at room temperature for 20 minutes. The reaction mixture was diluted with dichloromethane (2 mL) and purified with silica column chromatography (0% to 100% ethyl acetate in n-heptane) to afford benzyl 8-(5-(cyclopentylcarbamoyl)-5,6-dihydro-4H-pyrrolo[3,4-d]thiazol-2-yl)-3,8-diazabicyclo[3.2.1]octane-3-carboxylate (56 mg, 65%) as a white solid.

A solution of benzyl 8-(5-(cyclopentylcarbamoyl)-5,6-dihydro-4H-pyrrolo[3,4-d]thiazol-2-yl)-3,8-diazabicyclo[3.2.1]octane-3-carboxylate (54 mg, 0.11 mmol) in 33% hydrogen bromide in acetic acid (0.2 mL, 3.46 mmol) was stirred at room temperature for 30 minutes. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-N-cyclopentyl-4,6-dihydro-5H-pyrrolo[3,4-d]thiazole-5-carboxamide (4.9 mg, 12%, Example 68) as a white solid. ¹H-NMR: δ_{H} (400 MHz, CDCl₃, mixture of rotamers) 4.61-4.51 (2H, m), 4.41-4.33 (2H, m), 4.24-4.08 (4H, m), 3.29-3.19 (2H, m), 2.72-2.63 (2H, m), 2.15-2.00 (4H, m), 2.00-1.91 (2, m), 1.76-1.64 (2H, m), 1.64-1.58 (2H, m), 1.48-1.33 (2H, m); UPLC (Method C): t_{R} 3.68 min, 99%, MS (ESI) 348.2 (M+H)⁺.

The following example (69) was prepared using procedures analogous to Example 68, using the appropriate starting materials.

### Example 69: Synthesis of 2-(3,8-diazabicyclo[3.2.1]octan-8-yl)-N-(4,4-difluorocyclohexyl)-4,6-dihydro-5H-pyrrolo[3,4-d]thiazole-5-carboxamide

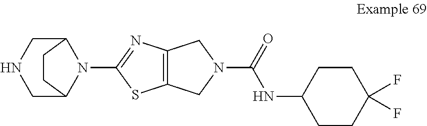 [SMILES: O=C(NC1CCC(F)(F)CC1)N1Cc2nc(N3C4CCC3CNC4)sc2C1]

Prepared using 1,1-difluoro-4-isocyanatocyclohexane as the isocyanate component.

¹H-NMR: δ_{H} (400 MHz, CDCl₃, mixture of rotamers) 4.64-4.49 (2H, m), 4.38 (2H, t, J 3.8), 4.23-4.11 (2H, m), 4.06 (1H, m), 3.86 (1H, m), 3.32-3.16 (2H, m), 2.73-2.63 (2H, m), 2.17-2.00 (6H, m), 2.00-1.78 (4H, m), 1.57-1.46 (2H, m); UPLC (Method C): t_{R} 3.90 min, 99%, MS (ESI) 398.2 (M+H)⁺.

### Example 70: Synthesis of 2-(3,8-diazabicyclo[3.2.1]octan-8-yl)-N-(4,4-difluorocyclohexyl)-6,7-dihydropyrazolo[1,5-a]pyrazine-5(4H)-carboxamide

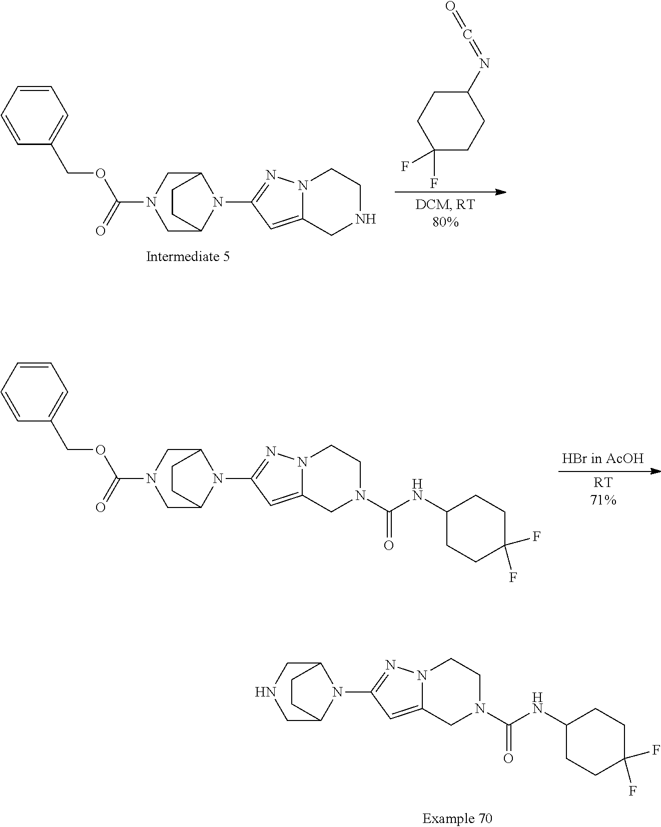 [SMILES: O=C(OCc1ccccc1)N1CC2CCC(C1)N2c1cc2n(n1)CCNC2 ; O=C=NC1CCC(F)(F)CC1 ; O=C(NC1CCC(F)(F)CC1)N1CCn2nc(N3C4CCC3CN(C(=O)OCc3ccccc3)C4)cc2C1 ; O=C(NC1CCC(F)(F)CC1)N1CCn2nc(N3C4CCC3CNC4)cc2C1]

To a solution of benzyl 8-(4,5,6,7-tetrahydropyrazolo[1,5-a]pyrazin-2-yl)-3,8-diazabicyclo[3.2.1]octane-3-carboxylate (170 mg, 0.46 mmol, Intermediate 5) in dichloromethane (2 mL) was added 1,1-difluoro-4-isocyanatocyclohexane (97 mg, 0.60 mmol) and the mixture was stirred at room temperature for 2 hours. The mixture was concentrated in vacuo, purified with preparative reversed phase chromatography (Waters XSelect™ CSH C18 column, ammonium bicarbonate in water/acetonitrile) and lyophilized to afford benzyl 8-(5-((4,4-difluorocyclohexyl) carbamoyl)-4,5,6,7-tetrahydropyrazolo[1,5-a]pyrazin-2-yl)-3,8-diazabicyclo[3.2.1]octane-3-carboxylate (200 mg, 80%) as a white solid.

A solution of benzyl 8-(5-((4,4-difluorocyclohexyl) carbamoyl)-4,5,6,7-tetrahydropyrazolo[1,5-a]pyrazin-2-yl)-3,8-diazabicyclo[3.2.1]octane-3-carboxylate (195 mg, 0.37 mmol) in 33% hydrogen bromide in acetic acid (1 mL, 17.4 mmol) was stirred at room temperature for 30 minutes. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-N-(4,4-difluorocyclohexyl)-6,7-dihydropyrazolo[1,5-a]pyrazine-5(4H)-carboxamide (104 mg, 71%, Example 70) as a white solid. ¹H-NMR: δ_{H} (400 MHz, CDCl₃, mixture of rotamers) 5.44 (1H, s), 4.49 (2H, s), 4.32 (1H, d, J 7.4), 4.03 (2H, t, J 5.4), 3.98-3.90 (2H, m), 3.89-3.75 (3H, m), 3.26-3.14 (2H, m), 2.67-2.55 (2H, m), 2.18-1.99 (4H, m), 1.99-1.76 (6H, m), 1.59-1.43 (2H, m); UPLC (Method C): t_{R} 3.63 min, 99%, MS (ESI) 395.2 (M+H)⁺.

### Example 71: Synthesis of 1-(2-(3,8-diazabicyclo[3.2.1]octan-8-yl)-6,7-dihydropyrazolo[1,5-a]pyrazin-5(4H)-yl)-2-(4,4-difluorocyclohexyl)ethan-1-one

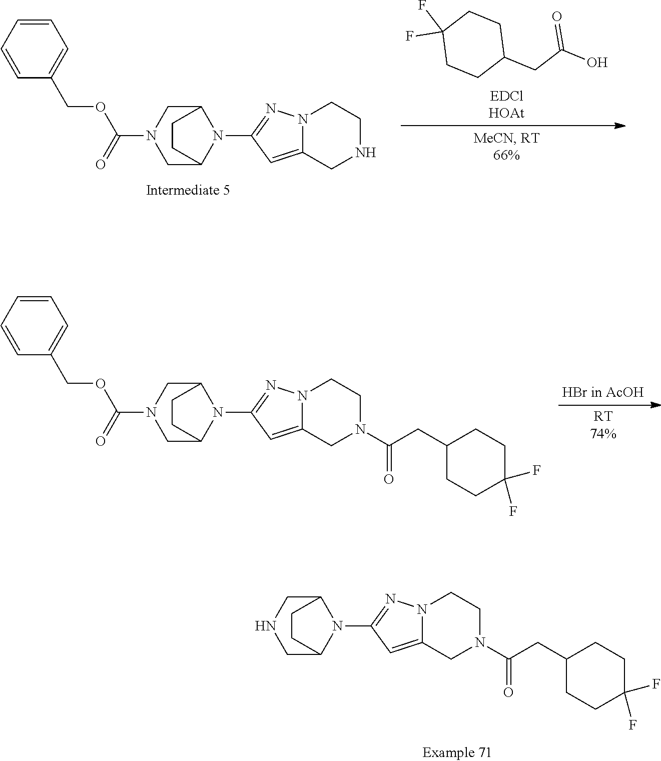 [SMILES: O=C(OCc1ccccc1)N1CC2CCC(C1)N2c1cc2n(n1)CCNC2 ; O=C(O)CC1CCC(F)(F)CC1 ; O=C(CC1CCC(F)(F)CC1)N1CCn2nc(N3C4CCC3CN(C(=O)OCc3ccccc3)C4)cc2C1 ; O=C(CC1CCC(F)(F)CC1)N1CCn2nc(N3C4CCC3CNC4)cc2C1]

To a solution of benzyl 8-(4,5,6,7-tetrahydropyrazolo[1,5-a]pyrazin-2-yl)-3,8-diazabicyclo[3.2.1]octane-3-carboxylate (170 mg, 0.46 mmol, Intermediate 5), 2-(4,4-difluorocyclohexyl)acetic acid (99 mg, 0.56 mmol) and triethylamine (0.10 mL, 0.69 mmol) in acetonitrile (2 mL) were added N-(3-dimethylaminopropyl)-N′-ethylcarbodiimide hydrochloride (106 mg, 0.56 mmol) and 1-hydroxy-7-azabenzotriazole (6.30 mg, 0.05 mmol). The mixture was stirred at room temperature for 5 hours. The mixture was concentrated, the residue was redissolved in methanol (3 mL), purified with preparative reversed phase chromatography (Phenomenex LUNA C18 column, formic acid in water/acetonitrile) and lyophilized to afford benzyl 8-(5-(2-(4,4-difluorocyclohexyl) acetyl)-4,5,6,7-tetrahydropyrazolo[1,5-a]pyrazin-2-yl)-3,8-diazabicyclo[3.2.1]octane-3-carboxylate (162 mg, 65%) as a white solid.

A solution of benzyl 8-(5-(2-(4,4-difluorocyclohexyl) acetyl)-4,5,6,7-tetrahydropyrazolo[1,5-a]pyrazin-2-yl)-3,8-diazabicyclo[3.2.1]octane-3-carboxylate (160 mg, 0.30 mmol) in 33% hydrogen bromide in acetic acid (1 mL, 17.3 mmol) was stirred at room temperature for 30 minutes. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydropyrazolo[1,5-a]pyrazin-5(4H)-yl)-2-(4,4-difluorocyclohexyl)ethan-1-one (89 mg, 74%, Example 71) as a white solid. ¹H-NMR: δ_{H} (400 MHz, CDCl₃, mixture of rotamers) 5.45 (1H, d, J7.9), 4.76-4.56 (2H, m), 4.10-3.97 (3H, m), 3.94 (2H, s), 3.88 (1H, t, J 5.4), 3.26-3.12 (2H, m), 2.61 (2H, d, J 12.1), 2.39-2.26 (2H, m), 2.17-1.91 (5H, m), 1.91-1.66 (6H, m), 1.39-1.22 (2H, m); UPLC (Method C): t_{R} 3.92 min, 99%, MS (ESI) 394.2 (M+H)⁺.

The following example (72) was prepared using procedures analogous to Example 71, using the appropriate starting materials.

### Example 72: Synthesis of 1-(2-(3,8-diazabicyclo[3.2.1]octan-8-yl)-6,7-dihydropyrazolo[1,5-a]pyrazin-5(4H)-yl)-2-(4-fluorophenoxy)ethan-1-one

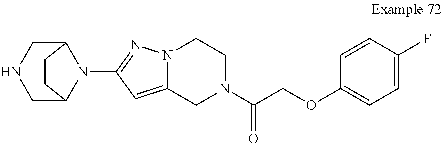 [SMILES: O=C(COc1ccc(F)cc1)N1CCn2nc(N3C4CCC3CNC4)cc2C1]

Prepared using 4-fluorophenoxyacetic acid as the acid component.

¹H-NMR: δ_{H} (400 MHz, CDCl₃, mixture of rotamers) 7.02-6.95 (2H, m), 6.93-6.86 (2H, m), 5.46 (1H, s), 4.73 (4H, d, J 5.8), 4.11-3.98 (4H, m), 3.93 (2H, s), 3.19 (2H, d, J 12.1), 2.66-2.54 (2H, m), 1.95 (2H, d, J 7.7), 1.83 (2H, d, J7.7); UPLC (Method A): 0.88 min, 99%, MS (ESI) 386.2 (M+H)⁺.

### Example 73: Synthesis of 1-(2-(3,8-diazabicyclo[3.2.1]octan-8-yl)-6,7-dihydrothiazolo[5,4-c]pyridin-5(4H)-yl)-2-((2-fluoropyridin-4-yl)oxy)ethan-1-one

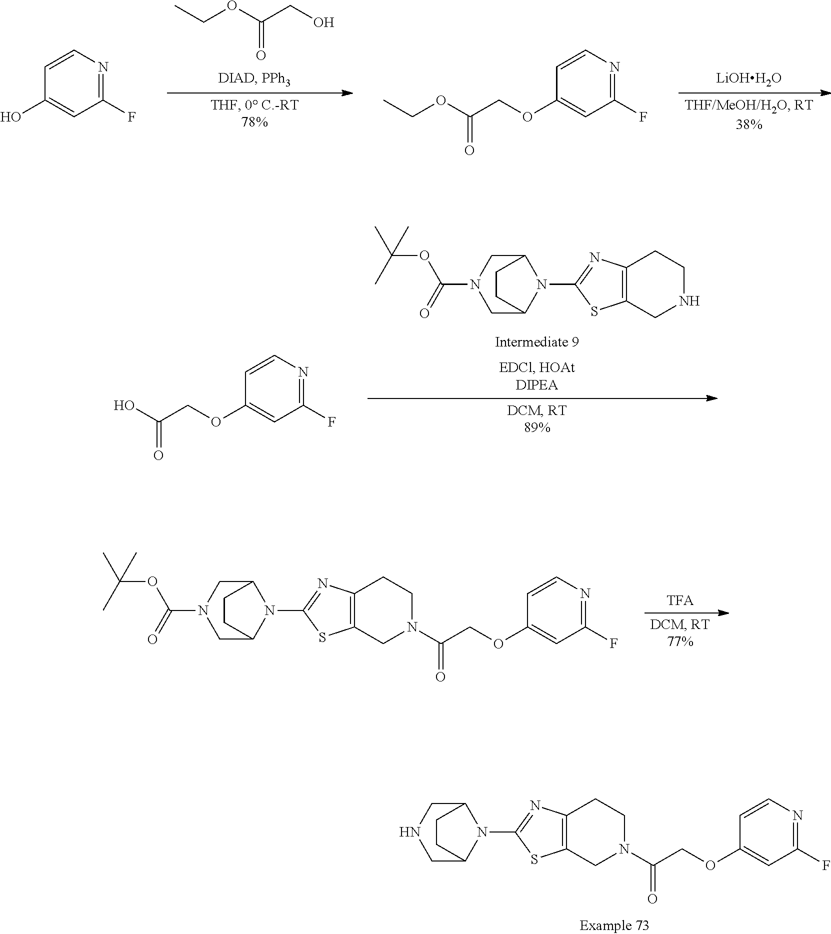

A solution of ethyl glycolate (1.05 mL, 11.1 mmol), 2-fluoro-4-hydroxypyridine (840 mg, 7.43 mmol) and triphenylphosphine (4.48 g, 17.1 mmol) in tetrahydrofuran (20 mL) was cooled to 0° C. Diisopropyl azodicarboxylate (3.32 mL, 17.1 mmol) was added dropwise and the mixture was stirred at 0° C. for 10 minutes, followed by stirring at room temperature for 16 hours. The mixture was concentrated in vacuo, the residue dissolved in water and extracted with ethyl acetate twice. The combined organic layers were washed with water twice and brine once. The organic layer was dried with sodium sulfate, concentrated in vacuo and triturated in isopropyl alcohol for 16 hours. The solids were filtered off, washed with isopropyl alcohol and the filtrate was concentrated in vacuo to afford a yellow oil. The oil was purified with silica column chromatography (0% to 100% ethyl acetate in n-heptane) to afford ethyl 2-((2-fluoropyridin-4-yl)oxy)acetate (1.3 g, 88%) as a light yellow oil.

A solution of lithium hydroxide monohydrate (544 mg, 13.0 mmol) in water (5 mL) was added to a solution of ethyl 2-((2-fluoropyridin-4-yl)oxy)acetate (1.3 g, 6.48 mmol) in tetrahydrofuran (5 mL) and methanol (5 mL) and the mixture was stirred at room temperature for 1 hour. The reaction was concentrated in vacuo, the residue was dissolved in water and washed with dichloromethane three times. The aqueous layer was acidified with 10% aqueous citric acid solution and was extracted with dichloromethane three times. The combined organic layers were concentrated in vacuo to afford a white solid. Additional material precipitated from the aqueous layer after 16 hours and was collected by filtration. Both solid batches were combined to afford 2-((2-fluoropyridin-4-yl)oxy)acetic acid (687 mg, 61%) as a white solid.

A solution of 2-((2-fluoropyridin-4-yl)oxy)acetic acid (56.4 mg, 0.33 mmol), N-(3-dimethylaminopropyl)-N′-ethylcarbodiimide hydrochloride (63.2 mg, 0.330 mmol) and 1-hydroxy-7-azabenzotriazole (4.08 mg, 0.030 mmol) in acetonitrile (3 mL) was stirred at room temperature for 5 minutes. Next, tert-butyl 8-(4,5,6,7-tetrahydrothiazolo[5,4-c]pyridin-2-yl)-3,8-diazabicyclo[3.2.1]octane-3-carboxylate (105 mg, 0.30 mmol, Intermediate 9) and N,N-diisopropylethylamine (0.06 mL, 0.33 mmol) were added and the mixture was stirred at room temperature for 16 hours. The mixture was concentrated in vacuo and the residue was purified with silica column chromatography (0% to 100% ethyl acetate in n-heptane) to afford tert-butyl 8-(5-(2-((2-fluoropyridin-4-yl)oxy) acetyl)-4,5,6,7-tetrahydrothiazolo[5,4-c]pyridin-2-yl)-3,8-diazabicyclo[3.2.1]octane-3-carboxylate (141 mg, 93%) as a white solid.

To a solution of tert-butyl 8-(5-(2-((2-fluoropyridin-4-yl)oxy) acetyl)-4,5,6,7-tetrahydrothiazolo[5,4-c]pyridin-2-yl)-3,8-diazabicyclo[3.2.1]octane-3-carboxylate (140 mg, 0.28 mmol) in dichloromethane (1 mL) was added trifluoroacetic acid (0.43 mL, 5.56 mmol) and the mixture was stirred at room temperature for 1 hour. The mixture was concentrated in vacuo and co-evaporated with toluene. The residue was dissolved in methanol (3.5 mL) and 7N ammonia in methanol (0.5 mL), purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-((2-fluoropyridin-4-yl)oxy)ethan-1-one (98 mg, 77%, Example 73) as a white solid. ¹H-NMR: δ_{H} (400 MHz, CDCl₃, mixture of rotamers) 8.06 (1H, d, J 5.9), 6.87-6.73 (1H, m), 6.55-6.40 (1H, m), 4.86-4.82 (2H, m), 4.63 (1.3H, d, J2.0), 4.54 (0.7H, d, J2.3), 4.13 (2H, s), 3.91 (0.7H, t, J 5.8), 3.79 (1.3H, t, J 5.8), 3.23-3.20 (2H, m), 2.83-2.57 (4H, m), 2.13-1.88 (4H, m); UPLC (Method A): t_{R} 1.58 min, 99%, MS (ESI) 404.2 (M+H)⁺.

The following example (74) was prepared using procedures analogous to Example 73, using the appropriate starting materials.

### Example 74: Synthesis of 1-(2-(3,8-diazabicyclo[3.2.1]octan-8-yl)-6,7-dihydrothiazolo[5,4-c]pyridin-5(4H)-yl)-2-((6-fluoropyridin-3-yl)oxy)ethan-1-one

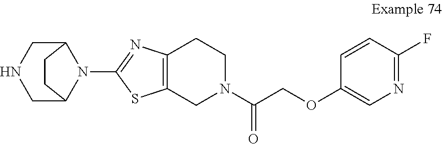 [SMILES: O=C(COc1ccc(F)nc1)N1CCc2nc(N3C4CCC3CNC4)sc2C1]

Prepared using 2-fluoro-5-hydroxypyridine as the starting reagent.

¹H-NMR: δ_{H} (400 MHz, CDCl₃, mixture of rotamers) 7.89 (1H, d, J 3.8), 7.49-7.38 (1H, m), 6.92-6.83 (1H, m), 4.81-4.78 (2H, m), 4.64 (1.3H, d, J 2.1), 4.56 (0.7H, d, J 2.0), 4.19-4.05 (2H, m), 3.91 (0.7H, t, J 5.8), 3.81 (1.3H, t, J 5.8), 3.23-3.20 (2H, m), 2.81-2.61 (4H, m), 2.13-1.88 (4H, m); UPLC (Method A): t_{R} 1.61 min, 99%, MS (ESI) 404.2 (M+H)⁺.

### Example 75: Synthesis of 2-cyclopentyl-1-(2-(3-methyl-3,8-diazabicyclo[3.2.1]octan-8-yl)-6,7-dihydrothiazolo[5,4-c]pyridin-5(4H)-yl)ethan-1-one

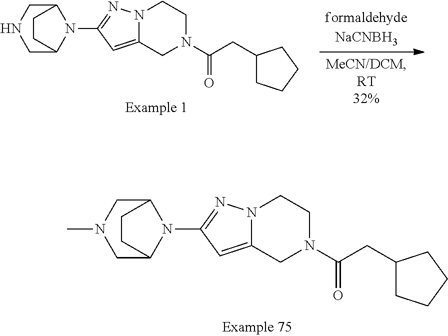 [SMILES: O=C(CC1CCCC1)N1CCn2nc(N3C4CCC3CNC4)cc2C1 ; CN1CC2CCC(C1)N2c1cc2n(n1)CCN(C(=O)CC1CCCC1)C2]

To a suspension of 1-(2-(3,8-diazabicyclo[3.2.1]octan-8-yl)-6,7-dihydrothiazolo[5,4-c]pyridin-5(4H)-yl)-2-cyclopentylethan-1-one (81 mg, 0.18 mmol, Example 1) in acetonitrile (2 mL) at room temperature was added 37% formaldehyde in water (0.02 mL, 0.28 mmol) and N,N-diisopropylethylamine (0.03 mL, 0.18 mmol) followed by dichloromethane (1 mL) to improve solubility. After 1 hour, sodium cyanoborohydride (17.3 mg, 0.28 mmol) was added and the mixture was stirred at room temperature for 3 days. The mixture was quenched with 7N ammonia in methanol (1 mL) and concentrated in vacuo. The residue was purified with reversed phase chromatography (Waters XSelect™ CSH C18 column, ammonium bicarbonate in water/acetonitrile followed by Phenomenex LUNA C18 column, formic acid in water/acetonitrile) and lyophilized to afford 2-cyclopentyl-1-(2-(3-methyl-3,8-diazabicyclo[3.2.1]octan-8-yl)-6,7-dihydrothiazolo[5,4-c]pyridin-5(4H)-yl)ethan-1-one (22.1 mg, 32% yield, Example 75) as a sticky off-white solid. ¹H-NMR: δ_{H} (400 MHz, DMSO-d₆) 4.50 (2H, m), 4.10 (2H, br s), 3.72 (2H, m), 2.62-2.54 (3H, m), 2.49-2.45 (1H, m), 2.45-2.36 (2H, m), 2.27 (2H, m), 2.20-2.06 (4H, m), 1.91-1.79 (4H, m), 1.79-1.67 (2H, m), 1.65-1.40 (4H, m), 1.20-1.03 (2H, m); UPLC (Method C): t_{R} 4.07 min, 97%, MS (ESI) 375.2 (M+H)⁺.

The following example (76) was prepared using procedures analogous to Example 75, using the appropriate starting materials.

### Example 76: Synthesis of 2-(4,4-difluorocyclohexyl)-1-(2-(3-methyl-3,8-diazabicyclo[3.2.1]octan-8-yl)-6,7-dihydrothiazolo[5,4-c]pyridin-5(4H)-yl)ethan-1-one

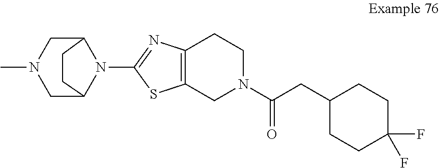 [SMILES: CN1CC2CCC(C1)N2c1nc2c(s1)CN(C(=O)CC1CCC(F)(F)CC1)CC2]

Prepared using Example 2 as starting reagent.

¹H-NMR: δ_{H} (400 MHz, DMSO-d₆) 4.57-4.44 (2H, m), 4.15-4.04 (2H, m), 3.78-3.66 (2H, m), 2.62-2.54 (3H, m), 2.49-2.45 (1H, m), 2.45-2.36 (2H, m), 2.27 (2H, d, J 10.7), 2.20-2.06 (4H, m), 1.91-1.79 (4H, m), 1.79-1.67 (2H, m), 1.65-1.40 (4H, m), 1.20-1.03 (2H, m); UPLC (Method C): t_{R} 4.15 min, 96%, MS (ESI) 425.4 (M+H)⁺.

### Example 77: Synthesis of N-(4,4-difluorocyclohexyl)-2-(3-methyl-3,8-diazabicyclo[3.2.1]octan-8-yl)-6,7-dihydrothiazolo[5,4-c]pyridine-5(4H)-carboxamide

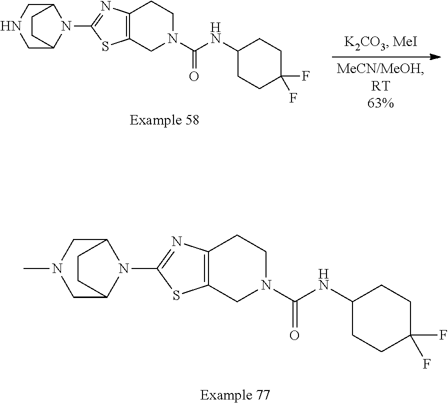 [SMILES: O=C(NC1CCC(F)(F)CC1)N1CCc2nc(N3C4CCC3CNC4)sc2C1 ; CN1CC2CCC(C1)N2c1nc2c(s1)CN(C(=O)NC1CCC(F)(F)CC1)CC2]

To a solution of 2-(3,8-diazabicyclo[3.2.1]octan-8-yl)-N-(4,4-difluorocyclohexyl)-6,7-dihydrothiazolo[5,4-c]pyridine-5(4H)-carboxamide (82 mg, 0.20 mmol, Example 58) and potassium carbonate (69 mg, 0.50 mmol) in acetonitrile (1 mL) and methanol (0.5 mL) was added methyl iodide (37 μL, 0.59 mmol). The mixture was stirred at room temperature for 90 minutes and concentrated in vacuo. The residue was purified with preparative reversed phase chromatography (Waters XSelect™ CSH C18 column, ammonium bicarbonate in water/acetonitrile) and lyophilized to afford N-(4,4-difluorocyclohexyl)-2-(3-methyl-3,8-diazabicyclo[3.2.1]octan-8-yl)-6,7-dihydrothiazolo[5,4-c]pyridine-5(4H)-carboxamide (54.0 mg, 63%, Example 77) as a white solid. ¹H-NMR: δ_{H} (400 MHz, CDCl₃) 4.42 (2H, s), 4.31 (1H, d, J 7.4), 4.16 (2H, s), 3.82 (1H, m), 3.65 (2H, t, J 5.7), 2.75-2.66 (2H, m), 2.66-2.57 (2H, m), 2.50-2.40 (2H, m), 2.22 (3H, s), 2.16-1.75 (10H, m), 1.54-1.46 (2H, m); UPLC (Method B): t_{R} 2.41 min, 99%, MS (ESI) 426.2 (M+H)⁺.

The following examples (78-80) were prepared using procedures analogous to Example 77, using the appropriate starting materials.

### Example 78: Synthesis of N-cyclopentyl-2-(3-methyl-3,8-diazabicyclo[3.2.1]octan-8-yl)-6,7-dihydrothiazolo[5,4-c]pyridine-5(4H)-carboxamide

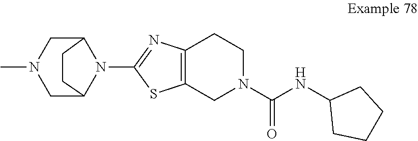 [SMILES: CN1CC2CCC(C1)N2c1nc2c(s1)CN(C(=O)NC1CCCC1)CC2]

Prepared using Example 50 as starting reagent.

¹H-NMR: δ_{H} (400 MHz, CDCl₃) 4.42-4.38 (3H, m), 4.21-4.05 (3H, m), 3.64 (2H, t, J 5.8), 2.75-2.66 (2H, m), 2.66-2.57 (2H, m), 2.51-2.40 (2H, m), 2.22 (3H, s), 2.09-1.91 (6H, m), 1.75-1.53 (4H, m), 1.43-1.29 (2H, m); UPLC (Method B): 2.32 min, 99%, MS (ESI) 376.2 (M+H)⁺.

### Example 79: Synthesis of 2-((2-fluoropyridin-4-yl)oxy)-1-(2-(3-methyl-3,8-diazabicyclo[3.2.1]octan-8-yl)-6,7-dihydrothiazolo[5,4-c]pyridin-5(4H)-yl)ethan-1-one

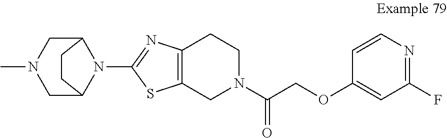 [SMILES: CN1CC2CCC(C1)N2c1nc2c(s1)CN(C(=O)COc1ccnc(F)c1)CC2]

Prepared using Example 73 as starting reagent.

¹H-NMR: δ_{H} (400 MHz, CDCl₃, mixture of rotamers) 8.06 (1H, d, J 5.9), 6.86-6.77 (1H, m), 6.53-6.42 (1H, m), 4.84 (2H, d, J 13.0), 4.64 (1.3H, d, J2.2), 4.54 (0,7H, d, J 2.2), 4.16 (2H, d, J 4.8), 4.00-3.87 (0.7H, m), 3.87-3.73 (1.3H, m), 2.84-2.69 (2H, m), 2.69-2.58 (2H, m), 2.46-2.42 (2H, m), 2.23 (3H, s), 2.06-1.91 (4H, m); UPLC (Method B): 1.59 min, 99%, MS (ESI) 418.2 (M+H)⁺.

### Example 80: Synthesis of 2-((6-fluoropyridin-3-yl)oxy)-1-(2-(3-methyl-3,8-diazabicyclo[3.2.1]octan-8-yl)-6,7-dihydrothiazolo[5,4-c]pyridin-5(4H)-yl)ethan-1-one

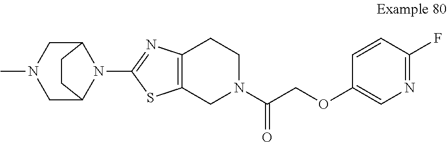 [SMILES: CN1CC2CCC(C1)N2c1nc2c(s1)CN(C(=O)COc1ccc(F)nc1)CC2]

Prepared using Example 74 as starting reagent.

¹H-NMR: δ_{H} (400 MHz, CDCl₃, mixture of rotamers) 7.94-7.82 (1H, m), 7.48-7.36 (1H, m), 6.91-6.80 (1H, m), 4.84-4.73 (2H, m), 4.66-4.58 (1.3H, m), 4.58-4.52 (0.7H, m), 4.23-4.09 (2H, m), 3.95-3.84 (0.7H, m), 3.84-3.74 (1.3H, m), 2.81-2.67 (2H, m), 2.67-2.56 (2H, m), 2.49-2.38 (2H, m), 2.23 (3H, s), 2.04-1.91 (4H, m); UPLC (Method B): 1.64 min, 99%, MS (ESI) 418.2 (M+H)⁺.

### Example 81: Synthesis of N-(4,4-difluorocyclohexyl)-2-(3-methyl-3,8-diazabicyclo[3.2.1]octan-8-yl)-4,6-dihydro-5H-pyrrolo[3,4-d]thiazole-5-carboxamide

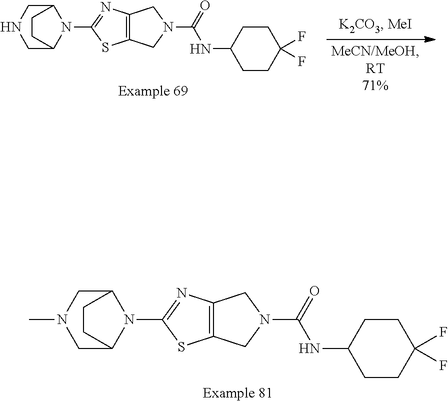 [SMILES: O=C(NC1CCC(F)(F)CC1)N1Cc2nc(N3C4CCC3CNC4)sc2C1 ; CN1CC2CCC(C1)N2c1nc2c(s1)CN(C(=O)NC1CCC(F)(F)CC1)C2]

To a solution of 2-(3,8-diazabicyclo[3.2.1]octan-8-yl)-N-(4,4-difluorocyclohexyl)-4,6-dihydro-5H-pyrrolo[3,4-d]thiazole-5-carboxamide (62 mg, 0.16 mmol, Example 69) and potassium carbonate (54 mg, 0.39 mmol) in acetonitrile (1 mL) and dichloromethane (1 mL) was added methyl iodide (29 μL, 0.46 mmol), The mixture was stirred at room temperature for 90 minutes and concentrated in vacuo. The residue was purified with preparative reversed phase chromatography (Waters XSelect™ CSH C18 column, ammonium bicarbonate in water/acetonitrile) and lyophilized to afford N-(4,4-difluorocyclohexyl)-2-(3-methyl-3,8-diazabicyclo[3.2.1]octan-8-yl)-4,6-dihydro-5H-pyrrolo[3,4-d]thiazole-5-carboxamide (46 mg, 71%, Example 81) as a white solid. ¹H-NMR: δ_{H} (400 MHz, CDCl₃) 4.64-4.50 (2H, m), 4.38 (2H, t, J 3.9), 4.26-4.14 (2H, m), 4.05 (1H, d, J7.7), 3.85 (1H, d, J9.2), 2.70-2.57 (2H, m), 2.53-2.42 (2H, m), 2.23 (3H, s), 2.17-1.78 (10H, m), 1.53 (2H, d, J 12.6); UPLC (Method B): t_{R} 2.41 min, 99%, MS (ESI) 412.2 (M+H)⁺

The following examples (82-84) were prepared using procedures analogous to Example 81, using the appropriate starting materials.

### Example 82: Synthesis of N-cyclopentyl-2-(3-methyl-3,8-diazabicyclo[3.2.1]octan-8-yl)-4,6-dihydro-5H-pyrrolo[3,4-d]thiazole-5-carboxamide

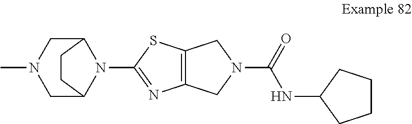 [SMILES: CN1CC2CCC(C1)N2c1nc2c(s1)CN(C(=O)NC1CCCC1)C2]

Prepared using Example 68 as starting reagent.

¹H-NMR: δ_{H} (400 MHz, CDCl₃, mixture of rotamers) 4.66-4.47 (2H, m), 4.47-4.31 (2H, m), 4.23-4.05 (4H, m), 2.70-2.54 (2H, m), 2.53-2.41 (2H, m), 2.23 (3H, s), 2.11-1.93 (6H, m), 1.75-1.60 (4H, m), 1.46-1.32 (2H, m); UPLC (Method B): 2.31 min, 99%, MS (ESI) 362.2 (M+H)⁺.

### Example 83: Synthesis of 2-cyclopentyl-1-(2-(3-methyl-3,8-diazabicyclo[3.2.1]octan-8-yl)-4,6-dihydro-5H-pyrrolo[3,4-d]thiazol-5-yl)ethan-1-one

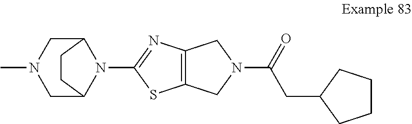 [SMILES: CN1CC2CCC(C1)N2c1nc2c(s1)CN(C(=O)CC1CCCC1)C2]

Prepared using Example 66 as starting reagent.

¹H-MR: δ_{H} (400 MHz, CDCl₃, mixture of rotamers) 4.73-4.60 (2H, m), 4.55-4.52 (2H, m), 4.19 (2H, s), 2.70-2.59 (2H, m), 2.55-2.43 (2H, m), 2.34 (3H, m), 2.23 (3H, s), 2.09-1.96 (4H, m), 1.88 (2H, m), 1.65-1.56 (4H, m), 1.26-1.16 (2H, m); UPLC (Method C): t_{R} 4.21 min, 99%, MS (ESI) 361.2 (M+H)⁺

### Example 84: Synthesis of 2-(4,4-difluorocyclohexyl)-1-(2-(3-methyl-3,8-diazabicyclo[3.2.1]octan-8-yl)-4,6-dihydro-5H-pyrrolo[3,4-d]thiazol-5-yl)ethan-1-one

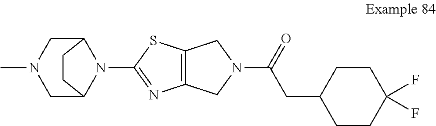 [SMILES: CN1CC2CCC(C1)N2c1nc2c(s1)CN(C(=O)CC1CCC(F)(F)CC1)C2]

Prepared using Example 67 as starting reagent.

¹H-NMR: δ_{H} (400 MHz, CDCl₃, mixture of rotamers) 4.72-4.59 (2H, m), 4.58-4.46 (2H, m), 4.19 (2H, br s), 2.71-2.56 (2H, m), 2.53-2.41 (2H, m), 2.29-2.18 (5H, m), 2.14-1.95 (7H, m), 1.95-1.67 (4H, m), 1.43-1.25 (2H, m); UPLC (Method C): 4.24 min, 99%, MS (ESI) 411.2 (M+H)⁺.

### Example 85: Synthesis of N-(4,4-difluorocyclohexyl)-2-(3-methyl-3,8-diazabicyclo[3.2.1]octan-8-yl)-6,7-dihydropyrazolo[1,5-a]pyrazine-5(4H)-carboxamide

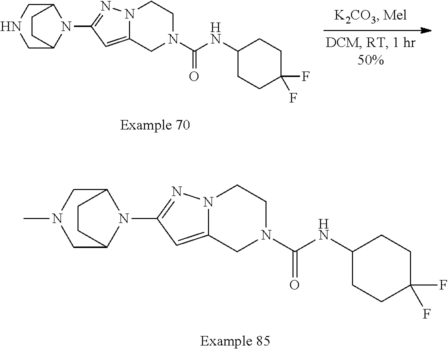 [SMILES: O=C(NC1CCC(F)(F)CC1)N1CCn2nc(N3C4CCC3CNC4)cc2C1 ; CN1CC2CCC(C1)N2c1cc2n(n1)CCN(C(=O)NC1CCC(F)(F)CC1)C2]

To a solution of 2-(3,8-diazabicyclo[3.2.1]octan-8-yl)-N-(4,4-difluorocyclohexyl)-6,7-dihydropyrazolo[1,5-a]pyrazine-5(4H)-carboxamide (70 mg, 0.18 mmol, Example 70) and potassium carbonate (61 mg, 0.44 mmol) in dichloromethane (1.5 mL) was added methyl iodide (33 μL, 0.53 mmol), The mixture was stirred at room temperature for 1 hour and concentrated in vacuo. The residue was purified with preparative reversed phase chromatography (Waters XSelect™ CSH C18 column, ammonium bicarbonate in water/acetonitrile) and lyophilized to afford N-(4,4-difluorocyclohexyl)-2-(3-methyl-3,8-diazabicyclo[3.2.1]octan-8-yl)-6,7-dihydropyrazolo[1,5-a]pyrazine-5(4H)-carboxamide (36 mg, 53%, Example 85) as a white solid. ¹H-NMR: δ_{H} (400 MHz, CDCl₃) 5.43 (1H, s), 4.49 (2H, s), 4.31 (1H, d, J7.4), 4.11-3.90 (4H, m), 3.84 (3H, t, J5.4), 2.70-2.52 (2H, m), 2.49-2.36 (2H, m), 2.21 (3H, s), 2.18-1.97 (4H, m), 1.97-1.76 (6H, m), 1.51 (2H, m); UPLC (Method B): t_{R} 2.10 min, 99%, MS (ESI) 409.4 (M+H)⁺.

The following example (86) was prepared using procedures analogous to Example 85, using the appropriate starting materials.

### Example 86: Synthesis of 2-(4,4-difluorocyclohexyl)-1-(2-(3-methyl-3,8-diazabicyclo[3.2.1]octan-8-yl)-6,7-dihydropyrazolo[1,5-a]pyrazin-5(4H)-yl)ethan-1-one

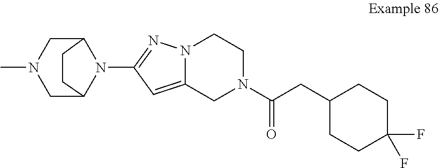 [SMILES: CN1CC2CCC(C1)N2c1cc2n(n1)CCN(C(=O)CC1CCC(F)(F)CC1)C2]

Prepared using Example 71 as starting reagent.

¹H-NMR: δ_{H} (400 MHz, CDCl₃) 5.45 (1H, d, J 8.1), 4.77-4.55 (2H, m), 4.10-3.94 (5H, m), 3.88 (1H, t, J5.5), 2.60 (2H, d, J 10.5), 2.42 (2H, d, J 10.5), 2.38-2.27 (2H, m), 2.22 (3H, s), 2.16-1.94 (3H, m), 1.94-1.65 (8H, m), 1.36-1.24 (2H, m); UPLC (Method B): t_{R} 2.29 min, 99%, MS (ESI) 408.2 (M+H)⁺.

### Example 97: Synthesis of 1-(2-(3,8-diazabicyclo[3.2.1]octan-8-yl)-5,6-dihydro-[1,2,4]triazolo[1,5-a]pyrazin-7 (8H)-yl)-2-(4,4-difluorocyclohexyl)ethan-1-one

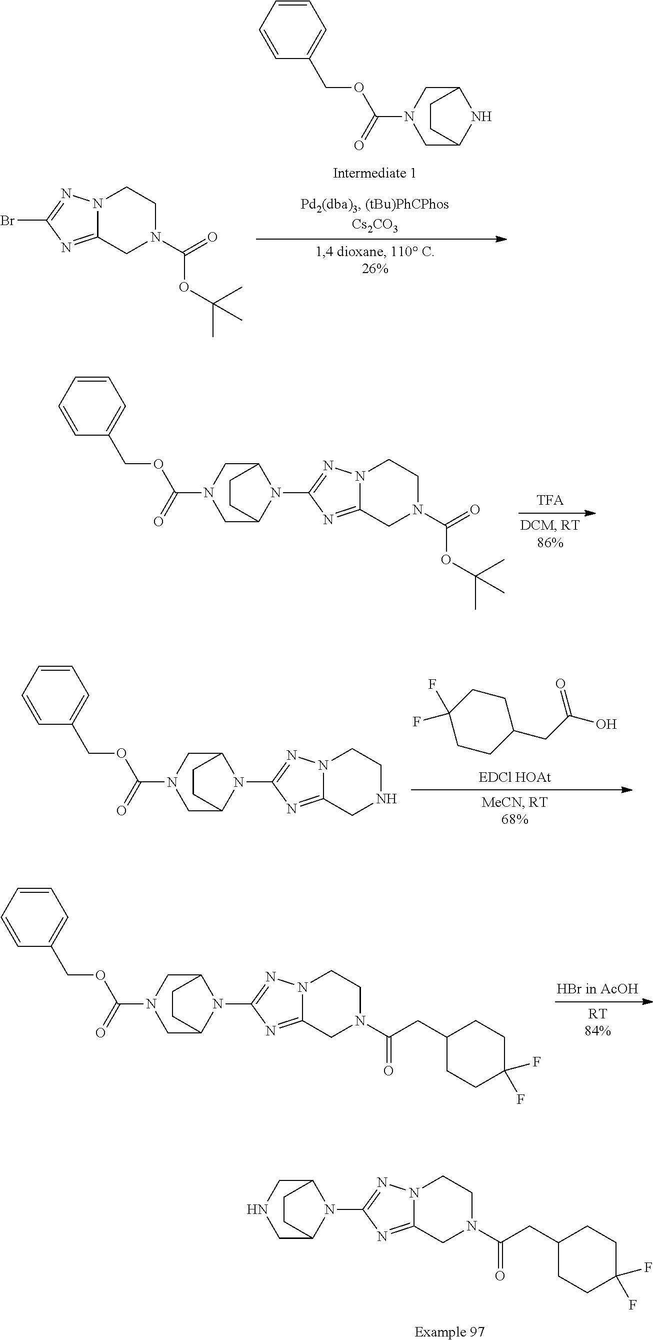

Under an argon atmosphere, (′Bu) PhCPhos (65.5 mg, 0.16 mmol) and Pd₂(dba)₃ (67 mg, 0.07 mmol) were suspended in 1,4-dioxane (1 mL) and heated to 80° C. for 5 minutes. The mixture was cooled to room temperature and transferred under an argon atmosphere to a stirring solution of tert-butyl 2-bromo-5,6-dihydro-[1,2,4]triazolo[1,5-a]pyrazine-7 (8H)-carboxylate (223 mg, 0.74 mmol), benzyl 3,8-diazabicyclo[3.2.1]octane-3-carboxylate (254 mg, 1.03 mmol, Intermediate 1) and cesium carbonate (599 mg, 1.84 mmol) in 1,4-dioxane (3 mL) and the mixture was heated to 100° C. for 16 hours. The mixture was filtered over Celite®, washed with acetonitrile and concentrated in vacuo. The residue was purified with silica column chromatography (0% to 100% ethyl acetate in n-heptane) to afford tert-butyl 2-(3-((benzyloxy)carbonyl)-3,8-diazabicyclo[3.2.1]octan-8-yl)-5,6-dihydro-[1,2,4]triazolo[1,5-a]pyrazine-7 (8H)-carboxylate (92 mg, 26%) as a white solid.

To a solution of tert-butyl 2-(3-((benzyloxy)carbonyl)-3,8-diazabicyclo[3.2.1]octan-8-yl)-5,6-dihydro-[1,2,4]triazolo[1,5-a]pyrazine-7 (8H)-carboxylate (92 mg, 0.20 mmol) in dichloromethane (2 mL) was added trifluoroacetic acid (0.30 mL, 3.93 mmol) and the mixture was stirred at room temperature for 1 hour. The mixture was concentrated in vacuo, partitioned between dichloromethane and saturated aqueous sodium carbonate solution. The layers were separated, and the aqueous layer was extracted with dichloromethane twice. The combined organic layers were washed with brine, dried with sodium sulfate and concentrated in vacuo to afford benzyl 8-(5,6,7,8-tetrahydro-[1,2,4]triazolo[1,5-a]pyrazin-2-yl)-3,8-diazabicyclo[3.2.1]octane-3-carboxylate (65 mg, 86%) as a yellow solid.

A solution of benzyl 8-(5,6,7,8-tetrahydro-[1,2,4]triazolo[1,5-a]pyrazin-2-yl)-3,8-diazabicyclo[3.2.1]octane-3-carboxylate (65 mg, 0.18 mmol), 2-(4,4-difluorocyclohexyl)acetic acid (34.6 mg, 0.19 mmol), triethylamine (0.04 mL, 0.27 mmol), N-(3-dimethylaminopropyl)-N′-ethylcarbodiimide hydrochloride (40.6 mg, 0.21 mmol) and 1-hydroxy-7-azabenzotriazole (2.4 mg, 0.02 mmol) in acetonitrile (2 mL) was stirred at room temperature for 16 hours. The mixture was concentrated in vacuo, purified with preparative reversed phase chromatography (Waters XSelect™ CSH C18 column, ammonium bicarbonate in water/acetonitrile) and lyophilized to afford benzyl 8-(7-(2-(4,4-difluorocyclohexyl)acetyl)-5,6,7,8-tetrahydro-[1,2,4]triazolo[1,5-a]pyrazin-2-yl)-3,8-diazabicyclo[3.2.1]octane-3-carboxylate (63 mg, 68%) as a white solid.

To a solution of benzyl 8-(7-(2-(4,4-difluorocyclohexyl)acetyl)-5,6,7,8-tetrahydro-[1,2,4]triazolo[1,5-a]pyrazin-2-yl)-3,8-diazabicyclo[3.2.1]octane-3-carboxylate (63 mg, 0.12 mmol) in acetic acid (1 mL) was added 33% hydrogen bromide in acetic acid (0.10 mL, 0.60 mmol) and the mixture was stirred at room temperature for 2 hours. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-5,6-dihydro-[1,2,4]triazolo[1,5-a]pyrazin-7(8H)-yl)-2-(4,4-difluorocyclohexyl)ethan-1-one (40 mg, 84%, Example 97) as a white solid. ¹H-NMR: δ_{H} (400 MHz, CDCl₃, mixture of rotamers) 4.85-4.60 (2H, m), 4.20 (2H, s), 4.11-4.08 (2H, m), 4.06-3.89 (2H, m), 3.23-3.10 (2H, m), 2.66-2.62 (2H, m), 2.39-2.28 (2H, m), 2.14-1.67 (1H, m), 1.32 (2H, m); UPLC (Method A): t_{R} 1.78 min, 99%, MS (ESI) 395.2 (M+H)⁺.

### Example 98: Synthesis of 1-(2-(3,8-diazabicyclo[3.2.1]octan-8-yl)-7,7-dimethyl-6,7-dihydrothiazolo[5,4-c]pyridin-5(4H)-yl)-2-(4,4-difluorocyclohexyl)ethan-1-one

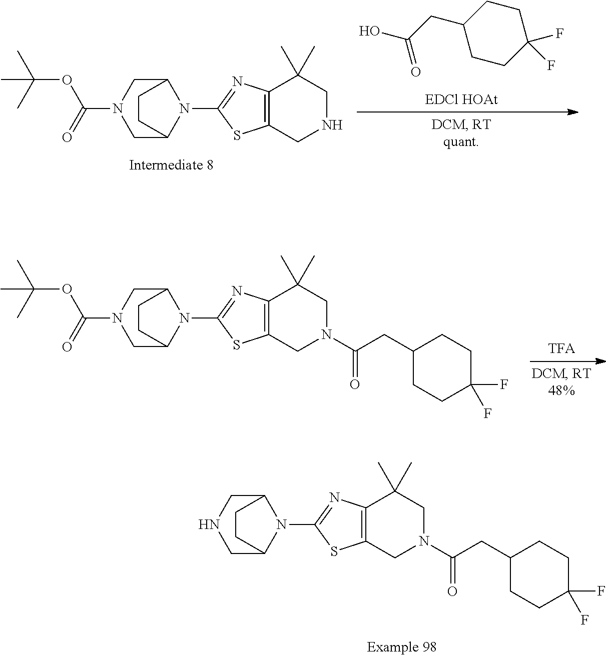 [SMILES: CC(C)(C)OC(=O)N1CC2CCC(C1)N2c1nc2c(s1)CNCC2(C)C ; O=C(O)CC1CCC(F)(F)CC1 ; CC(C)(C)OC(=O)N1CC2CCC(C1)N2c1nc2c(s1)CN(C(=O)CC1CCC(F)(F)CC1)CC2(C)C ; CC1(C)CN(C(=O)CC2CCC(F)(F)CC2)Cc2sc(N3C4CCC3CNC4)nc21]

A solution of 2-(4,4-difluorocyclohexyl)acetic acid (51.8 mg, 0.29 mmol), 1-hydroxy-7-azabenzotriazole (3.60 mg, 0.03 mmol) and N-(3-dimethylaminopropyl)-N′-ethylcarbodiimide hydrochloride (55.7 mg, 0.29 mmol) in dichloromethane (3 mL) was stirred at room temperature for 10 minutes. Next, tert-butyl 8-(7,7-dimethyl-4,5,6,7-tetrahydrothiazolo[5,4-c]pyridin-2-yl)-3,8-diazabicyclo[3.2.1]octane-3-carboxylate (100 mg, 0.26 mmol, Intermediate 8) and N,N-diisopropylethylamine (0.06 mL, 0.32 mmol) were added and the mixture was stirred at room temperature for 16 hours. The mixture was concentrated in vacuo and purified with silica column chromatography (0% to 100% ethyl acetate in n-heptane) to afford tert-butyl 8-(5-(2-(4,4-difluorocyclohexyl)acetyl)-7,7-dimethyl-4,5,6,7-tetrahydrothiazolo[5,4-c]pyridin-2-yl)-3,8-diazabicyclo[3.2.1]octane-3-carboxylate (172 mg, quantitative) as a white solid.

To a solution of tert-butyl 8-(5-(2-(4,4-difluorocyclohexyl)acetyl)-7,7-dimethyl-4,5,6,7-tetrahydrothiazolo[5,4-c]pyridin-2-yl)-3,8-diazabicyclo[3.2.1]octane-3-carboxylate (172 mg, 0.32 mmol) in dichloromethane (1 mL) was added trifluoroacetic acid (0.49 mL, 6.39 mmol) and the mixture was stirred at room temperature for 2 hours. The mixture was concentrated in vacuo, redissolved in methanol and 7N ammonia in methanol was added. The resulting mixture was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-7,7-dimethyl-6,7-dihydrothiazolo[5,4-c]pyridin-5(4H)-yl)-2-(4,4-difluorocyclohexyl)ethan-1-one (67 mg, 48%, Example 98) as a white solid. ¹H-NMR: δ_{H} (400 MHz, CDCl₃, mixture of rotamers) 4.66-4.46 (2H, m), 4.16-4.07 (2H, m), 3.65-3.38 (2H, m,), 3.22 (2H, d, J 12.3), 2.69-2.59 (2H, m), 2.38-2.30 (2H, m), 2.15-1.95 (5H, m), 1.95-1.66 (6H, m), 1.33-1.25 (2H, m), 1.23 (6H, d, J 14.8); UPLC (Method A): t_{R} 2.96 min, 99%, MS (ESI) 439.2 (M+H)⁺.

The following examples (99-101) were prepared using procedures analogous to Example 98, using the appropriate starting materials.

### Example 99: Synthesis of 1-(2-(3,8-diazabicyclo[3.2.1]octan-8-yl)-7,7-dimethyl-6,7-dihydrothiazolo[5,4-c]pyridin-5(4H)-yl)-2-cyclopentylethan-1-one

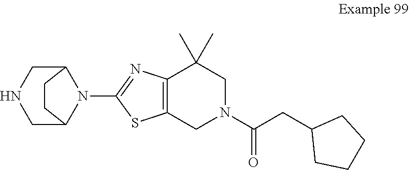 [SMILES: CC1(C)CN(C(=O)CC2CCCC2)Cc2sc(N3C4CCC3CNC4)nc21]

Prepared using 2-cyclopentylacetic acid as the acid compound.

¹H-NMR: δ_{H} (400 MHz, CDCl₃, mixture of rotamers) 4.68-4.45 (2H, m), 4.21-4.06 (2H, m), 3.66-3.40 (2H, m), 3.29-3.17 (2H, m), 2.72-2.55 (2H, m), 2.48-2.40 (2H, m), 2.40-2.22 (1H, m), 2.13-1.99 (2H, m), 1.95-1.80 (4H, m), 1.68-1.62 (2H, m), 1.59-1.51 (2H, m), 1.29-1.08 (8H, m); UPLC (Method A): t_{R} 2.95 min, 98%, MS (ESI) 389.2 (M+H)⁺.

### Example 100: Synthesis of 2-(4,4-difluorocyclohexyl)-1-(7,7-dimethyl-2-(piperazin-1-yl)-6,7-dihydrothiazolo[5,4-c]pyridin-5(4H)-yl)ethan-1-one

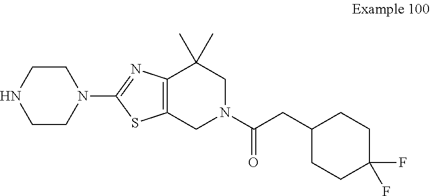 [SMILES: CC1(C)CN(C(=O)CC2CCC(F)(F)CC2)Cc2sc(N3CCNCC3)nc21]

Prepared using 2-(4,4-difluorocyclohexyl)acetic acid as the acid compound.

¹H-NMR: δ_{H} (400 MHz, CDCl₃, mixture of rotamers) 4.65-4.45 (2H, m), 3.65-3.29 (6H, m), 3.01-2.87 (4H, m), 2.39-2.26 (2H, m), 2.14-1.93 (3H, m), 1.91-1.66 (4H, m), 1.39-1.12 (8H, m); UPLC (Method A): t_{R} 2.74 min, 99%, MS (ESI) 413.2 (M+H)⁺.

### Example 101: Synthesis of 2-cyclopentyl-1-(7,7-dimethyl-2-(piperazin-1-yl)-6,7-dihydrothiazolo[5,4-c]pyridin-5(4H)-yl)ethan-1-one

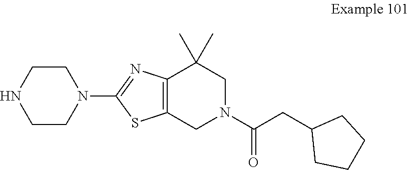 [SMILES: CC1(C)CN(C(=O)CC2CCCC2)Cc2sc(N3CCNCC3)nc21]

Prepared using 2-cyclopentylacetic acid as the acid compound.

¹H-NMR: δ_{H} (400 MHz, CDCl₃, mixture of rotamers) 4.65-4.46 (2H, m), 3.65-3.32 (6H, m), 3.02-2.91 (4H, m), 2.43 (2H, t, J 6.4), 2.39-2.21 (1H, m), 1.94-1.79 (2H, m), 1.69-1.61 (2H, dm), 1.59-1.50 (2H, m), 1.25-1.20 (6H, m), 1.20-1.11 (2H, m); UPLC (Method A): t_{R} 2.73 min, 99%, MS (ESI) 363.2 (M+H)⁺.

### Example 102: Synthesis of 2-cyclopentyl-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one

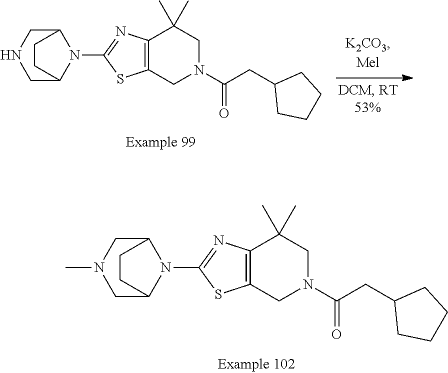 [SMILES: CC1(C)CN(C(=O)CC2CCCC2)Cc2sc(N3C4CCC3CNC4)nc21 ; CN1CC2CCC(C1)N2c1nc2c(s1)CN(C(=O)CC1CCCC1)CC2(C)C]

A solution of 1-(2-(3,8-diazabicyclo[3.2.1]octan-8-yl)-7,7-dimethyl-6,7-dihydrothiazolo[5,4-c]pyridin-5(4H)-yl)-2-cyclopentylethan-1-one (41.0 mg, 0.11 mmol, Example 99), potassium carbonate (36.5 mg, 0.26 mmol) and methyl iodide (0.02 mL, 0.32 mmol) in dichloromethane (2 mL) was stirred at room temperature for 16 hours. The mixture was filtered over Celite® washed with dichloromethane and concentrated in vacuo. The residue was purified with preparative reversed phase chromatography (Waters XSelect™ CSH C18 column, ammonium bicarbonate in water/acetonitrile) and lyophilized to afford 2-cyclopentyl-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one (23 mg, 53%, Example 102) as a beige solid. ¹H-NMR: δ_{H} (400 MHz, CDCl₃, mixture of rotamers) 4.66-4.46 (2H, m), 4.21-4.09 (2H, m), 3.63-3.38 (2H, m), 2.67-2.56 (2H, m), 2.49-2.40 (4H, m), 2.40-2.25 (1H, m), 2.23-2.20 (3H, m), 1.97-1.94 (4H, m), 1.92-1.81 (2H, m), 1.69-1.63 (2H, m), 1.59-1.49 (2H, m), 1.27-1.11 (8H, m); UPLC (Method A): t_{R} 1.83 min, 99%, MS (ESI) 403.2 (M+H)⁺.

The following examples (103-104) were prepared using procedures analogous to Example 102, using the appropriate starting materials.

### Example 103: Synthesis of 2-(4,4-difluorocyclohexyl)-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one

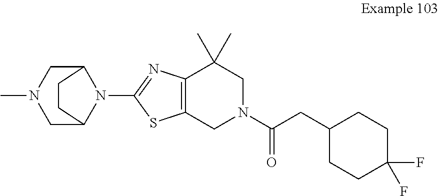 [SMILES: CN1CC2CCC(C1)N2c1nc2c(s1)CN(C(=O)CC1CCC(F)(F)CC1)CC2(C)C]

Prepared using Example 98 as starting reagent.

¹H-NMR: δ_{H} (400 MHz, CDCl₃, mixture of rotamers) 4.66-4.44 (2H, m), 4.16 (2H, d, J 4.5), 3.65-3.38 (2H, m), 2.68-2.56 (2H, m), 2.44 (2H, d, J 10.9), 2.39-2.28 (2H, m), 2.22 (3H, d, J 2.3), 2.14-2.01 (3H, m), 1.96-1.93 (4H, m), 1.92-1.67 (4H, m), 1.36-1.33 (2H, m), 1.28-1.21 (6H, m); UPLC (Method A): 1.83 min, 100%, MS (ESI) 453.2 (M+H)⁺.

### Example 104: Synthesis of 2-(4,4-difluorocyclohexyl)-1-(7,7-dimethyl-2-(4-methylpiperazin-1-yl)-6,7-dihydrothiazolo[5,4-c]pyridin-5(4H)-yl)ethan-1-one

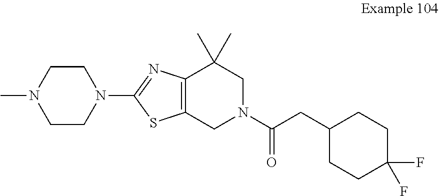 [SMILES: CN1CCN(c2nc3c(s2)CN(C(=O)CC2CCC(F)(F)CC2)CC3(C)C)CC1]

Prepared using Example 100 as starting reagent.

¹H-NMR: δ_{H} (400 MHz, CDCl₃, mixture of rotamers) 4.66-4.46 (2H, m), 3.64-3.36 (6H, m), 2.53-2.47 (4H, m), 2.39-2.28 (5H, m), 2.16-1.93 (3H, m), 1.93-1.65 (4H, m), 1.36-1.33 (2H, m), 1.29-1.23 (6H, m); UPLC (Method A): 1.74 min, 99%, MS (ESI) 427.2 (M+H)⁺.

### Example 107: Synthesis of 2-(3,8-diazabicyclo[3.2.1]octan-8-yl)-5-((cyclopentylmethyl)sulfonyl)-4,5,6,7-tetrahydrothiazolo[5,4-c]pyridine

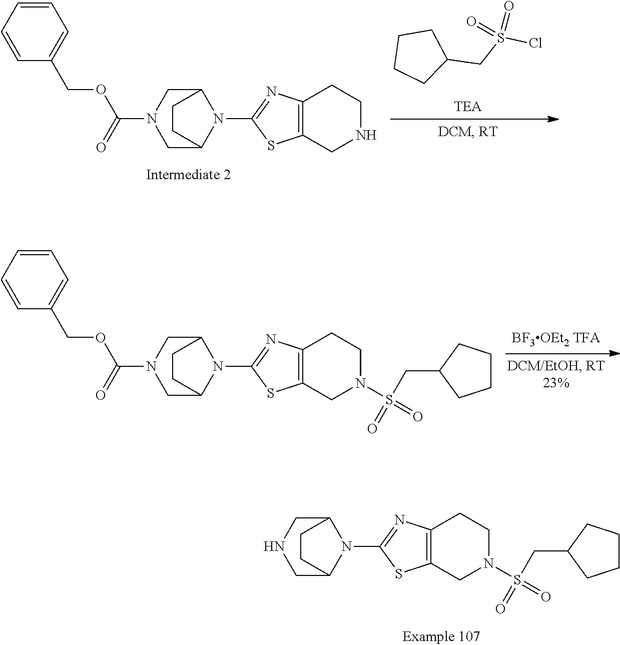 [SMILES: O=C(OCc1ccccc1)N1CC2CCC(C1)N2c1nc2c(s1)CNCC2 ; O=S(=O)(Cl)CC1CCCC1 ; O=C(OCc1ccccc1)N1CC2CCC(C1)N2c1nc2c(s1)CN(S(=O)(=O)CC1CCCC1)CC2 ; O=S(=O)(CC1CCCC1)N1CCc2nc(N3C4CCC3CNC4)sc2C1]

To a solution of benzyl 8-(4,5,6,7-tetrahydrothiazolo[5,4-c]pyridin-2-yl)-3,8-diazabicyclo[3.2.1]octane-3-carboxylate (100 mg, 0.26 mmol, Intermediate 2) and triethylamine (72.5 μL, 0.52 mmol) in dichloromethane was added cyclopentylmethanesulfonyl chloride (71 mg, 0.39 mmol) and the mixture was stirred at room temperature for 1 hour. The mixture was concentrated in vacuo. The residue was purified with preparative reversed phase chromatography (Waters XSelect™ CSH C18 column, ammonium bicarbonate in water/acetonitrile) and lyophilized to afford benzyl 8-(5-((cyclopentylmethyl)sulfonyl)-4,5,6,7-tetrahydrothiazolo[5,4-c]pyridin-2-yl)-3,8-diazabicyclo[3.2.1]octane-3-carboxylate (245 mg, 0.46 mmol) as a yellow solid.

To a solution of boron trifluoride etherate (2 mL, 15.8 mmol) and trifluoroacetic acid (1 mL, 13.1 mmol) in ethanol (0.3 mL) was added benzyl 8-(5-((cyclopentylmethyl)sulfonyl)-4,5,6,7-tetrahydrothiazolo[5,4-c]pyridin-2-yl)-3,8-diazabicyclo[3.2.1]octane-3-carboxylate (138 mg, 0.26 mmol) in dichloromethane (21 mL). The mixture was stirred at room temperature for 2 hours. The mixture was poured into saturated aqueous sodium carbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-5-((cyclopentylmethyl)sulfonyl)-4,5,6,7-tetrahydrothiazolo[5,4-c]pyridine (25 mg, 23%, Example 107) as a pink solid. ¹H-NMR: δ_{H} (400 MHz, CDCl₃) 4.39 (2H, t, J 2.0), 4.13 (2H, dd, J 4.4, 2.3), 3.64 (2H, t, J 5.8), 3.22 (2H, dd, J 12.4, 1.8), 2.97 (2H, d, J 7.0), 2.81-2.72 (2H, m), 2.67 (2H, dd, J 12.5, 2.2), 2.41-2.28 (1H, m), 2.12-2.01 (2H, m), 2.01-1.90 (4H, m), 1.70-1.62 (2H, m), 1.58-1.51 (2H, m), 1.35-1.21 (2H, m); UPLC (Method A): t_{R} 1.65 min, 94%, MS (ESI) 397.2 (M+H)⁺.

### Example 108: Synthesis of 2-(3,8-diazabicyclo[3.2.1]octan-8-yl)-N-cyclopentyl-6,7-dihydrothiazolo[5,4-c]pyridine-5(4H)-sulfonamide

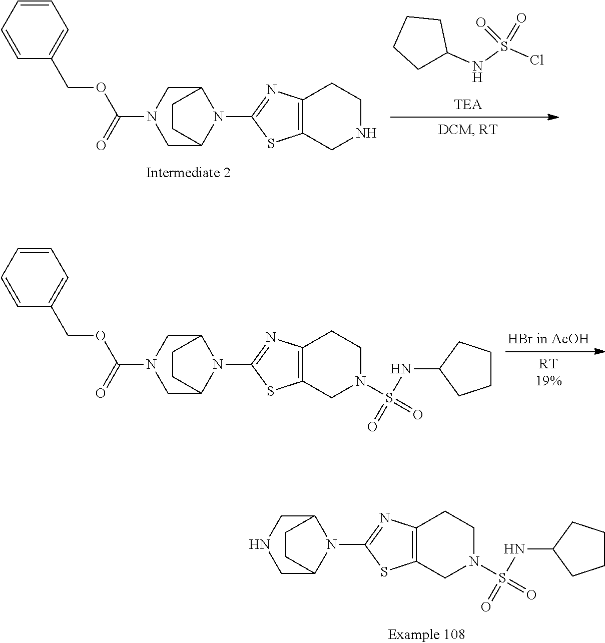 [SMILES: O=C(OCc1ccccc1)N1CC2CCC(C1)N2c1nc2c(s1)CNCC2 ; O=S(=O)(Cl)NC1CCCC1 ; O=C(OCc1ccccc1)N1CC2CCC(C1)N2c1nc2c(s1)CN(S(=O)(=O)NC1CCCC1)CC2 ; O=S(=O)(NC1CCCC1)N1CCc2nc(N3C4CCC3CNC4)sc2C1]

To a solution of benzyl 8-(4,5,6,7-tetrahydrothiazolo[5,4-c]pyridin-2-yl)-3,8-diazabicyclo[3.2.1]octane-3-carboxylate (100 mg, 0.26 mmol, Intermediate 2) and triethylamine (0.07 mL, 0.52 mmol) in dichloromethane (2 mL) was added cyclopentylsulfamoyl chloride (52.5 mg, 0.29 mmol) and the mixture was stirred at room temperature for 16 hours. The mixture was concentrated in vacuo to afford benzyl 8-(5-(N-cyclopentylsulfamoyl)-4,5,6,7-tetrahydrothiazolo[5,4-c]pyridin-2-yl)-3,8-diazabicyclo[3.2.1]octane-3-carboxylate (160 mg) as a brown gum, which was used in the next step without further purification.

A solution of benzyl 8-(5-(N-cyclopentylsulfamoyl)-4,5,6,7-tetrahydrothiazolo[5,4-c]pyridin-2-yl)-3,8-diazabicyclo[3.2.1]octane-3-carboxylate (160 mg, 0.30 mmol) in 33% hydrogen bromide in acetic acid (1.5 mL, 26.0 mmol) was stirred at room temperature for 16 hours. The mixture was slowly poured into diethyl ether and the resulting suspension was filtered off and quickly rinsed with diethyl ether. The residue was dissolved in saturated aqueous sodium bicarbonate solution and extracted with ethyl acetate twice. The combined organic layers were dried with sodium sulfate and concentrated in vacuo. The residue was purified with preparative reversed phase chromatography (Waters XSelect™ CSH C18 column, ammonium bicarbonate in water/acetonitrile) and lyophilized to afford 2-(3,8-diazabicyclo[3.2.1]octan-8-yl)-N-cyclopentyl-6,7-dihydrothiazolo[5,4-c]pyridine-5(4H)-sulfonamide (24 mg, 19%, Example 108) as a light-yellow solid. ¹H-NMR: δ_{H} (400 MHz, CDCl₃) 4.31 (2H, t, J 2.0), 4.15-4.11 (2H, m), 3.74-3.63 (1H, m), 3.57 (2H, t, J 5.8), 3.22 (2H, dd, J 12.4, 1.8), 2.81-2.73 (2H, m), 2.67 (2H, dd, J 12.3, 2.3), 2.11-1.90 (6H, m), 1.73-1.41 (7H, m); UPLC (Method A): t_{R} 2.08 min, 94%, MS (ESI) 398.2 (M+H)⁺.

### Example 109: Synthesis of 2-cyclopentyl-1-(2-(piperazin-1-yl)-6,7-dihydrothiazolo[5,4-c]pyridin-5(4H)-yl)ethan-1-one

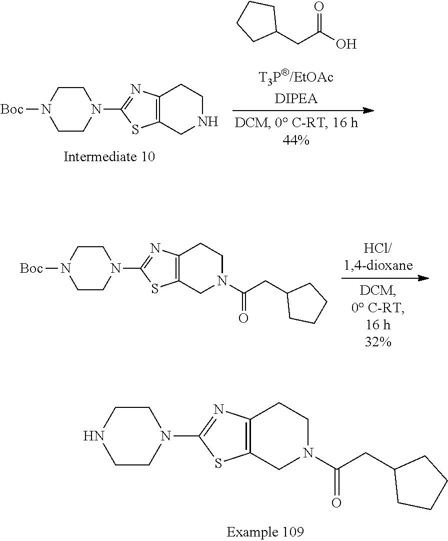 [SMILES: O=C(O)CC1CCCC1 ; CC(C)(C)OC(=O)N1CCN(c2nc3c(s2)CNCC3)CC1 ; CC(C)(C)OC(=O)N1CCN(c2nc3c(s2)CN(C(=O)CC2CCCC2)CC3)CC1 ; O=C(CC1CCCC1)N1CCc2nc(N3CCNCC3)sc2C1]

To a stirred solution of tert-butyl 4-(4,5,6,7-tetrahydrothiazolo[5,4-c]pyridin-2-yl)piperazine-1-carboxylate (1.70 g, 5.24 mmol, Intermediate 10) in DCM (30 mL) at 0° C. were added n-propylphosphonic acid anhydride, cyclic trimer (50% in EtOAc; 6.24 mL, 10.5 mmol), DIPEA (4.56 mL, 26.2 mmol) and 2-cyclopentylacetic acid (0.67 g, 5.24 mmol). The mixture was stirred at r.t. for 16 h, then diluted with DCM (100 mL) and washed with water (50 mL) and brine (50 mL). The organic layer was separated, dried over anhydrous sodium sulfate and concentrated under reduced pressure. The residue was purified by flash chromatography, eluting with 40% ethyl acetate in petroleum ether, to afford tert-butyl 4-(5-(2-cyclopentylacetyl)-4,5,6,7-tetrahydrothiazolo[5,4-c]pyridin-2-yl)piperazine-1-carboxylate (1.0 g, 44%) as an off-white solid.

To a stirred solution of tert-butyl 4-(5-(2-cyclopentylacetyl)-4,5,6,7-tetrahydrothiazolo[5,4-c]pyridin-2-yl)piperazine-1-carboxylate (1.0 g, 2.30 mmol) in DCM (10 mL) at 0° C. was added HCl (4 M in dioxane; 2.30 mL, 9.20 mmol). The reaction mixture was stirred at r.t. for 16 h, then concentrated under reduced pressure. The residue was dissolved in water, slowly basified with saturated sodium bicarbonate solution, stirred at r.t. for 15 min, then extracted with 10% methanol in DCM (2×50 mL). The combined organic layer was washed with water (25 mL) and brine (25 mL), dried over anhydrous sodium sulfate and concentrated under reduced pressure. The residue was purified by preparative HPLC using ammonium bicarbonate as a buffer to afford 2-cyclopentyl-1-(2-(piperazin-1-yl)-6,7-dihydrothiazolo[5,4-c]pyridin-5(4H)-yl)ethan-1-one (245 mg, 32%, Example 109) as an off-white solid. ¹H-NMR: δ_{H} (400 MHz, DMSO-d₆) 4.53-4.48 (2H, m), 3.72-3.48 (2H, m), 3.37-3.27 (4H, m), 2.83-2.81 (4H, m), 2.59-2.51 (2H, m), 2.43-2.37 (2H, m), 2.17-2.10 (1H, m), 1.75-1.62 (2H, m), 1.58-1.50 (4H, m), 1.13-1.08 (2H, m).

The following example (110) was prepared using procedures analogous to Example 109, using the appropriate starting materials.

### Example 110: Synthesis of 2-(4-fluorophenoxy)-1-(2-(piperazin-1-yl)-6,7-dihydrothiazolo[5,4-c]pyridin-5(4H)-yl)ethan-1-one

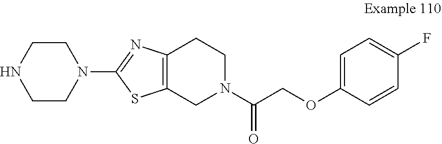 [SMILES: O=C(COc1ccc(F)cc1)N1CCc2nc(N3CCNCC3)sc2C1]

Prepared using 2-(4-fluorophenoxy)acetic acid as the acid component. ¹H-NMR: δ_{H} (400 MHz, DMSO-d₆) 7.02-6.97 (4H, m), 4.90-4.85 (2H, m), 4.65-4.63 (2H, m), 3.92-3.84 (2H, m), 3.45-3.42 (4H, m), 2.98-2.95 (4H, m), 2.77-2.66 (2H, m).

### Example 116: Synthesis of (S)-2-cyclopentyl-1-(2-(3-(hydroxymethyl)piperazin-1-yl)-6,7-dihydrothiazolo[5,4-c]pyridin-5(4H)-yl)ethan-1-one

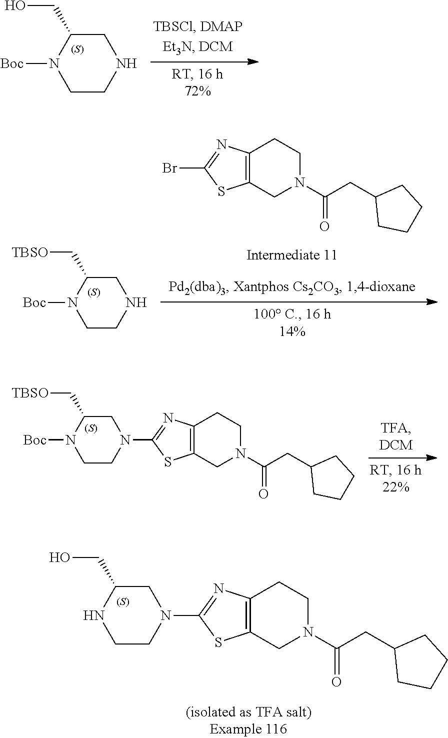

To a stirred solution of tert-butyl (S)-2-(hydroxymethyl)piperazine-1-carboxylate (200 mg, 0.925 mmol) in DCM (20 mL) at 0° C. under nitrogen were added TBDMS-Cl (1.05 g, 6.94 mmol) and triethylamine (0.258 mL, 1.85 mmol) followed by DMAP (11.3 mg, 0.092 mmol). The reaction mixture was stirred at r.t. for 16 h, then diluted with saturated sodium bicarbonate solution (20 mL) and extracted with 10% MeOH in DCM (2×20 mL). The combined organic extract was washed with brine (20 mL), dried over anhydrous sodium sulfate, filtered and concentrated under reduced pressure to afford tert-butyl (S)-2-(((tert-butyldimethylsilyl)oxy)methyl)piperazine-1-carboxylate (300 mg, 72%).

To a stirred solution of 1-(2-bromo-6,7-dihydrothiazolo[5,4-c]pyridin-5(4H)-yl)-2-cyclopentylethan-1-one (Intermediate 11; 200 mg, 0.607 mmol) in 1,4-dioxane (5 mL) under continuous bubbling of nitrogen were added tert-butyl (S)-2-(((tert-butyldimethylsilyl)oxy)methyl)piperazine-1-carboxylate (402 mg, 1.22 mmol), Cs₂CO₃ (594 mg, 1.82 mmol) and Xantphos (70.3 mg, 0.121 mmol). After 5 minutes, Pd₂(dba)₃ (55.6 mg, 0.061 mmol) was added. The mixture was stirred at 100° C. for 16 h, then quenched with ice-cold water (50 mL) and extracted with ethyl acetate (3×50 mL). The combined organic extract was washed with brine (30 mL), dried over anhydrous sodium sulfate, filtered and concentrated under reduced pressure. The residue was purified by flash chromatography, eluting with 50% ethyl acetate in petroleum ether, to obtain tert-butyl (S)-2-(((tert-butyldimethylsilyl)oxy)methyl)-4-(5-(2-cyclopentylacetyl)-4,5,6,7-tetrahydrothiazolo[5,4-c]pyridin-2-yl)piperazine-1-carboxylate (130 mg, 14%).

To a stirred solution of tert-butyl (S)-2-(((tert-butyldimethylsilyl)oxy)methyl)-4-(5-(2-cyclopentylacetyl)-4,5,6,7-tetrahydrothiazolo[5,4-c]pyridin-2-yl)piperazine-1-carboxylate (130 mg, 0.225 mmol) in DCM (5 mL) at r.t. was added TFA (2.81 mL, 11.2 mmol). The reaction mixture was stirred at r.t. for 16 h, then concentrated under reduced pressure. The residue was purified by preparative HPLC using TFA buffer to afford(S)-2-cyclopentyl-1-(2-(3-(hydroxymethyl)piperazin-1-yl)-6,7-dihydrothiazolo[5,4-c]pyridin-5(4H)-yl)ethan-1-one, trifluoroacetate salt (18.0 mg, 22%, Example 116) as a gummy solid. ¹H-NMR: δ_{H} (400 MHz, DMSO-d₆) 9.15-9.05 (1H, br. m), 8.79-8.68 (1H, br. m), 5.71-5.32 (1H, br. m), 4.57-4.52 (2H, m), 3.97-3.84 (2H, m), 3.75-3.57 (4H, m), 3.42-3.07 (5H, m), 2.63-2.54 (2H, m), 2.44-2.37 (2H, m), 2.22-2.09 (1H, m), 1.80-1.69 (2H, m), 1.64-1.43 (4H, m), 1.19-1.06 (2H, m).

The following example (117) may be prepared using procedures analogous to Example 116, using the appropriate starting materials.

### Example 117: Synthesis of (R)-2-cyclopentyl-1-(2-(3-(hydroxymethyl)piperazin-1-yl)-6,7-dihydrothiazolo[5,4-c]pyridin-5(4H)-yl)ethan-1-one

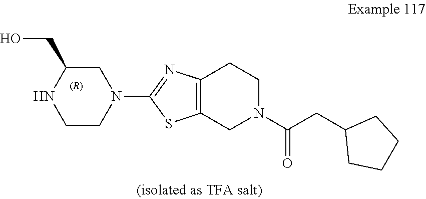

Example 117 may be prepared using tert-butyl (R)-2-(hydroxymethyl)piperazine-1-carboxylate instead of tert-butyl (S)-2-(hydroxymethyl)piperazine-1-carboxylate. The title compound was isolated as a trifluoroacetate salt. ¹H-NMR: δ_{H} (400 MHz, DMSO-d₆) 9.17-9.07 (1H, br. m), 8.80-8.68 (1H, br. m), 5.58-5.38 (1H, br. m), 4.57-4.52 (2H, m), 3.97-3.84 (2H, m), 3.75-3.57 (4H, m), 3.42-3.07 (5H, m), 2.63-2.54 (2H, m), 2.44-2.37 (2H, m), 2.22-2.09 (1H, m), 1.80-1.69 (2H, m), 1.64-1.43 (4H, m), 1.19-1.06 (2H, m).

### Example 118: Synthesis of 2-cyclopentyl-1-(2-(piperidin-4-yl)-6,7-dihydrothiazolo[5,4-c]pyridin-5(4H)-yl)ethan-1-one

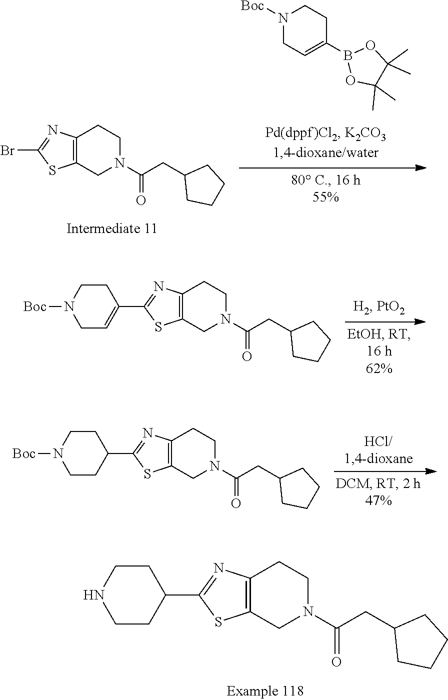 [SMILES: CC(C)(C)OC(=O)N1CC=C(B2OC(C)(C)C(C)(C)O2)CC1 ; O=C(CC1CCCC1)N1CCc2nc(Br)sc2C1 ; CC(C)(C)OC(=O)N1CC=C(c2nc3c(s2)CN(C(=O)CC2CCCC2)CC3)CC1 ; CC(C)(C)OC(=O)N1CCC(c2nc3c(s2)CN(C(=O)CC2CCCC2)CC3)CC1 ; O=C(CC1CCCC1)N1CCc2nc(C3CCNCC3)sc2C1]

To a stirred solution of 1-(2-bromo-6,7-dihydrothiazolo[5,4-c]pyridin-5(4H)-yl)-2-cyclopentylethan-1-one (500 mg, 1.52 mmol, Intermediate 11) in 1,4-dioxane (5 mL) and water (1 mL) under continuous bubbling of nitrogen were added tert-butyl 4-(4,4,5,5-tetramethyl-1,3,2-dioxaborolan-2-yl)-3,6-dihydropyridine-1 (2H)-carboxylate (470 mg, 1.52 mmol), potassium carbonate (420 mg, 3.04 mmol) and PdCl₂(dppf)-CH₂Cl₂ complex (111 mg, 0.152 mmol). The reaction mixture was heated to 100° C. and stirred for 16 h, then concentrated under reduced pressure, diluted with water (20 mL) and extracted with EtOAc (2×20 mL). The combined organic extract was washed with brine (20 mL), dried over anhydrous sodium sulfate, filtered and concentrated under reduced pressure. The residue was purified by flash chromatography, eluting with 40% ethyl acetate in hexane, to afford tert-butyl 4-(5-(2-cyclopentylacetyl)-4,5,6,7-tetrahydrothiazolo[5,4-c]pyridin-2-yl)-3,6-dihydropyridine-1 (2H)-carboxylate (400 mg, 55%) as an off-white solid.

To a stirred solution of tert-butyl 4-(5-(2-cyclopentylacetyl)-4,5,6,7-tetrahydrothiazolo[5,4-c]pyridin-2-yl)-3,6-dihydropyridine-1 (2H)-carboxylate (700 mg, 1.62 mmol) in ethanol (20 mL) under continuous bubbling of nitrogen was added platinum (IV) oxide (368 mg, 1.62 mmol). The mixture was stirred at r.t. under H₂ bladder pressure for 16 h, then filtered through Celite®, washing with methanol (20 mL) and concentrated under reduced pressure to obtain crude tert-butyl 4-(5-(2-cyclopentylacetyl)-4,5,6,7-tetrahydrothiazolo[5,4-c]pyridin-2-yl)piperidine-1-carboxylate (620 mg, 62%) as a brown solid, which was taken forward to the next step without further purification.

To a stirred solution of tert-butyl 4-(5-(2-cyclopentylacetyl)-4,5,6,7-tetrahydrothiazolo[5,4-c]pyridin-2-yl)piperidine-1-carboxylate (600 mg, 1.38 mmol) in DCM (25 mL) at r.t. was added HCl in dioxane (4 M; 0.692 mL, 2.77 mmol). The mixture was stirred at r.t. for 2 h, then concentrated under reduced pressure. The residue was purified by preparative HPLC using ammonium bicarbonate as buffer to afford 2-cyclopentyl-1-(2-(piperidin-4-yl)-6,7-dihydrothiazolo[5,4-c]pyridin-5(4H)-yl)ethan-1-one (420 mg, 47%, Example 118) as an off-white solid. ¹H-NMR: δ_{H} (400 MHz, DMSO-d₆) 4.72-4.64 (2H, m), 3.77-3.74 (2H, m), 3.03-3.00 (3H, m), 2.80-2.68 (1H, m), 2.67-2.60 (2H, m), 2.50-2.33 (3H, m), 2.33 (1H, m), 1.93-1.90 (2H, m), 1.77-1.72 (2H, m), 1.55-1.48 (6H, m), 1.14-1.11 (2H, m).

### Example 119: Synthesis of 1-(2-(3,8-diazabicyclo[3.2.1]octan-8-yl)-6,7-dihydrothiazolo[5,4-c]pyridin-5(4H)-yl)-2-(3,3-dimethylcyclobutyl)ethan-1-one

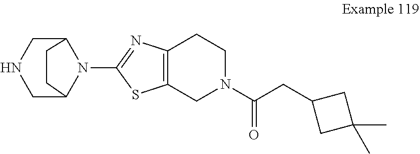 [SMILES: CC1(C)CC(CC(=O)N2CCc3nc(N4C5CCC4CNC5)sc3C2)C1]

Prepared according to the method of Example 1 using 2-(3,3-dimethylcyclobutyl)acetic acid as the acid component.

¹H-NMR: δ_{H} (400 MHz, CDCl₃, mixture of rotamers) 4.59 (1.3H, s), 4.47 (0.7H, s), 4.14 (2H, br s), 3.87 (0.7H, t, J 5.8), 3.72 (1.3H, t, J 5.8), 3.23 (2H, d, J 12.2), 2.76-2.61 (5H, m), 2.50 (2H, dd, J 16.1, 7.4), 2.11-2.03 (2H, m), 2.02-1.91 (4H, m), 1.54-1.44 (2H, m), 1.16-1.03 (6H, m); UPLC (Method A): t_{R} 1.72 min, 99%, MS (ESI) 375.2 (M+H)⁺.

### Example 120: Synthesis of (2-(3,8-diazabicyclo[3.2.1]octan-8-yl)-6,7-dihydrothiazolo[5,4-c]pyridin-5(4H)-yl)(2,2-dimethylpyrrolidin-1-yl)methanone

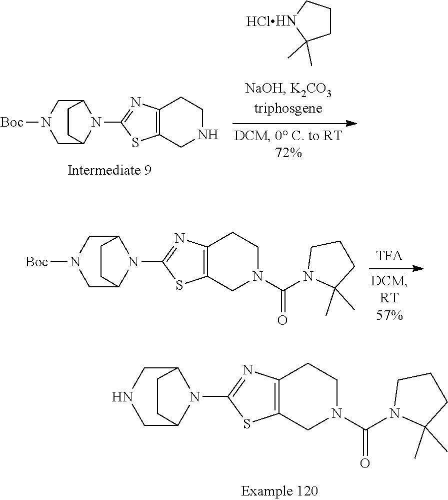

To a solution of tert-butyl 8-(4,5,6,7-tetrahydrothiazolo[5,4-c]pyridin-2-yl)-3,8-diazabicyclo[3.2.1]octane-3-carboxylate (100 mg, 0.29 mmol, Intermediate 9) in dichloromethane (1 mL) at 0° C. were added potassium carbonate (59 mg, 0.43 mmol) and triphosgene (34 mg, 0.11 mmol). The mixture was stirred at room temperature for 2 hours. Next, 2,2-dimethylpyrrolidine hydrochloride (77 mg, 0.57 mmol) and 1M aqueous sodium hydroxide solution (1.5 mL, 1.50 mmol) were added and the mixture was stirred at room temperature for 16 hours. The mixture was partitioned between dichloromethane and water. The layers were separated and the aqueous layer was extracted with dichloromethane twice. The combined organic layers were concentrated, purified with preparative reversed phase chromatography (Waters XSelect™ CSH C18 column, ammonium bicarbonate in water/acetonitrile) and co-evaporated with dichloromethane and n-heptrane to afford tert-butyl 8-(5-(2,2-dimethylpyrrolidine-1-carbonyl)-4,5,6,7-tetrahydrothiazolo[5,4-c]pyridin-2-yl)-3,8-diazabicyclo[3.2.1]octane-3-carboxylate (107 mg, 72%) as a yellow solid.

To a solution of tert-butyl 8-(5-(2,2-dimethylpyrrolidine-1-carbonyl)-4,5,6,7-tetrahydrothiazolo[5,4-c]pyridin-2-yl)-3,8-diazabicyclo[3.2.1]octane-3-carboxylate (106 mg, 0.22 mmol) in dichloromethane (1 mL) was added trifluoroacetic acid (0.34 mL, 4.46 mmol) and the mixture was stirred at room temperature for 1 hour. The mixture was concentrated in vacuo, purified with preparative reversed phase chromatography (Waters XSelect™ CSH C18 column, ammonium bicarbonate in water/acetonitrile) and lyophilized to afford (2-(3,8-diazabicyclo[3.2.1]octan-8-yl)-6,7-dihydrothiazolo[5,4-c]pyridin-5(4H)-yl)(2,2-dimethylpyrrolidin-1-yl)methanone (48 mg, 58%, Example 120) as a white solid. ¹H-NMR: δ_{H} (400 MHz, CDCl₃) 4.21-4.16 (2H, m), 4.16-4.09 (2H, m), 3.55-3.47 (2H, m), 3.47-3.40 (2H, m), 3.29-3.17 (2H, m), 2.75-2.69 (2H, m), 2.69-2.61 (2H, m), 2.12-2.00 (2H, m), 1.96-1.80 (4H, m), 1.80-1.71 (2H, m), 1.44 (6H, s); UPLC (Method A): t_{R} 2.30 min, 100%, MS (ESI) 376.2 (M+H)⁺.

The following examples (121-125) were prepared using procedures analogous to Example 120, using the appropriate starting materials.

### Example 121: Synthesis of (2-(3,8-diazabicyclo[3.2.1]octan-8-yl)-6,7-dihydrothiazolo[5,4-c]pyridin-5(4H)-yl)(3,3-dimethylpyrrolidin-1-yl)methanone

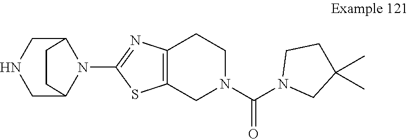 [SMILES: CC1(C)CCN(C(=O)N2CCc3nc(N4C5CCC4CNC5)sc3C2)C1]

Prepared using 3,3-dimethylpyrrolidine hydrochloride as the amine component.

¹H-NMR: δ_{H} (400 MHz, CDCl₃) 4.36-4.22 (2H, m), 4.20-4.08 (2H, m), 3.59-3.44 (4H, m), 3.29-3.19 (2H, m), 3.14 (2H, s), 2.80-2.60 (4H, m), 2.14-2.02 (2H, m), 1.99-1.89 (2H, m), 1.64 (2H, t, J 7.1), 1.07 (6H, s); UPLC (Method A): t_{R} 2.30 min, 100%, MS (ESI) 376.2 (M+H)⁺.

### Example 122: Synthesis of (2-(3,8-diazabicyclo[3.2.1]octan-8-yl)-6,7-dihydrothiazolo[5,4-c]pyridin-5(4H)-yl)(2-azaspiro[4.4]nonan-2-yl)methanone

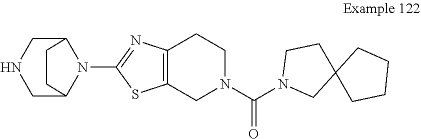 [SMILES: O=C(N1CCc2nc(N3C4CCC3CNC4)sc2C1)N1CCC2(CCCC2)C1]

Prepared using 2-azaspiro[4.4]nonane as the amine component.

¹H-NMR: δ_{H} (400 MHz, CDCl₃) 4.35-4.23 (2H, m), 4.18-4.06 (2H, m), 3.58-3.51 (2H, m), 3.51-3.43 (2H, m), 3.28-3.17 (4H, m), 2.78-2.69 (2H, m), 2.69-2.60 (2H, m), 2.12-2.02 (2H, m), 1.97-1.86 (2H, m), 1.79-1.70 (2H, m), 1.70-1.66 (2H, m), 1.64-1.46 (6H, m); UPLC (Method A): t_{R} 2.72 min, 98%, MS (ESI) 402.4 (M+H)⁺.

### Example 123: Synthesis of (2-(3,8-diazabicyclo[3.2.1]octan-8-yl)-6,7-dihydrothiazolo[5,4-c]pyridin-5(4H)-yl)(6-azaspiro[3.4]octan-6-yl)methanone

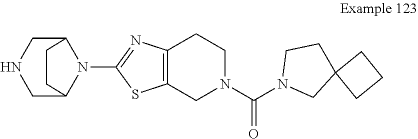 [SMILES: O=C(N1CCc2nc(N3C4CCC3CNC4)sc2C1)N1CCC2(CCC2)C1]

Prepared using 6-azaspiro[3.4]octane hydrochloride as the amine component.

¹H-NMR: δ_{H} (400 MHz, CDCl₃) 4.32-4.25 (2H, m), 4.18-4.09 (2H, m), 3.58-3.47 (2H, m), 3.46-3.38 (2H, m), 3.35 (2H, s), 3.28-3.17 (2H, m), 2.79-2.70 (2H, m), 2.70-2.61 (2H, m), 2.10-2.02 (2H, m), 2.02-1.96 (2H, m), 1.96-1.88 (6H, m), 1.88-1.82 (2H, m); UPLC (Method A): t_{R} 2.57 min, 99%, MS (ESI) 388.4 (M+H)⁺.

### Example 124: Synthesis of 2-(3,8-diazabicyclo[3.2.1]octan-8-yl)-N-(1-methylcyclobutyl)-6,7-dihydrothiazolo[5,4-c]pyridine-5(4H)-carboxamide

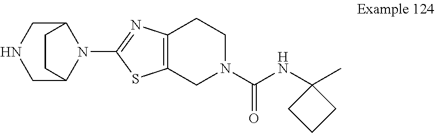 [SMILES: CC1(NC(=O)N2CCc3nc(N4C5CCC4CNC5)sc3C2)CCC1]

Prepared using 1-methylcyclobutan-1-amine hydrochloride as the amine component.

¹H-NMR: δ_{H} (400 MHz, CDCl₃) 4.57 (1H, br s), 4.39 (2H, s), 4.13 (2H, br s), 3.63 (2H, t, J5.7), 3.23 (2H, dd, J 12.5, 2.0), 2.74-2.63 (4H, m), 2.30-2.19 (2H, m), 2.10-1.99 (4H, m), 1.98-1.91 (2H, m), 1.90-1.76 (4H, m), 1.48 (3H, s); UPLC (Method A): t_{R} 1.28 min, 99%, MS (ESI) 362.2 (M+H)⁺.

### Example 125: Synthesis of 2-(3,8-diazabicyclo[3.2.1]octan-8-yl)-N-(1-methylcyclopentyl)-6,7-dihydrothiazolo[5,4-c]pyridine-5(4H)-carboxamide

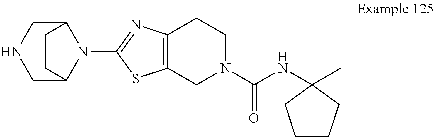 [SMILES: CC1(NC(=O)N2CCc3nc(N4C5CCC4CNC5)sc3C2)CCCC1]

Prepared using 1-methylcyclopentan-1-amine hydrochloride as the amine component.

¹H-NMR: δ_{H} (400 MHz, CDCl₃) 4.44 (1H, br s), 4.39 (2H, t, J 2.0), 4.16-4.09 (2H, m), 3.62 (2H, t, J 5.7), 3.23 (2H, dd, J 12.4, 1.8), 2.72-2.64 (4H, m), 2.09-2.00 (2H, m), 1.98-1.88 (4H, m), 1.76-1.65 (5H, m), 1.42 (3H, s); UPLC (Method A): t_{R} 1.46 min, 100%, MS (ESI) 376.2 (M+H)⁺.

The following examples (126-128) were prepared using procedures analogous to Example 71, using the appropriate starting materials.

### Example 126: Synthesis of 1-(2-(3,8-diazabicyclo[3.2.1]octan-8-yl)-6,7-dihydropyrazolo[1,5-a]pyrazin-5(4H)-yl)-2,2-difluoro-2-phenylethan-1-one

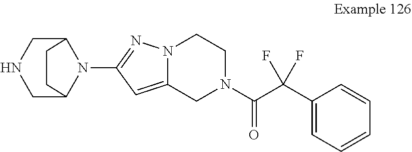 [SMILES: O=C(N1CCn2nc(N3C4CCC3CNC4)cc2C1)C(F)(F)c1ccccc1]

Prepared using 2,2-difluoro-2-phenylacetic acid as the acid component.

¹H-NMR: δ_{H} (400 MHz, CDCl₃, mixture of rotamers) 7.61-7.41 (5H, m), 5.54-5.24 (1H, m), 4.89-4.58 (2H, m), 4.23-3.74 (6H, m), 3.22-3.11 (2H, m), 2.65-2.55 (2H, m), 2.01-1.77 (4H, m); UPLC (Method A): 2.14 min, 99%, MS (ESI) 388.2 (M+H)⁺.

### Example 127: Synthesis of 1-(2-(3,8-diazabicyclo[3.2.1]octan-8-yl)-6,7-dihydropyrazolo[1,5-a]pyrazin-5(4H)-yl)-2-cyclopentylethan-1-one

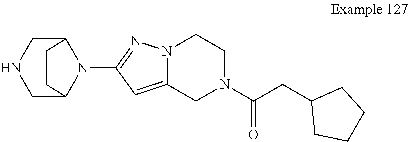 [SMILES: O=C(CC1CCCC1)N1CCn2nc(N3C4CCC3CNC4)cc2C1]

Prepared using cyclopentylacetic acid as the acid component.

¹H-NMR: δ_{H} (400 MHz, CDCl₃, mixture of rotamers) 5.45 (1H, d, J 5.5), 4.77-4.55 (2H, m), 4.09-3.85 (6H, m), 3.23-3.15 (2H, m), 2.65-2.56 (2H, m), 2.43 (2H, dd, J 13.9, 7.2), 2.34-2.20 (1H, m), 2.03-1.78 (6H, m), 1.69-1.49 (5H, m), 1.23-1.09 (2H, m); UPLC (Method A): 1.95 min, 99%, MS (ESI) 344.2 (M+H)⁺.

### Example 128: Synthesis of 1-(2-(3,8-diazabicyclo[3.2.1]octan-8-yl)-6,7-dihydropyrazolo[1,5-a]pyrazin-5(4H)-yl)-2-(1-methylcyclopentyl)ethan-1-one

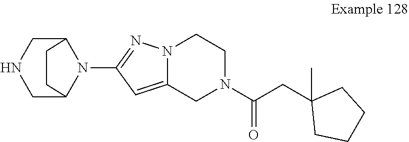 [SMILES: CC1(CC(=O)N2CCn3nc(N4C5CCC4CNC5)cc3C2)CCCC1]

Prepared using 2-(1-methylcyclopentyl)acetic acid as the acid component.

¹H-NMR: δ_{H} (400 MHz, CDCl₃, mixture of rotamers) 5.45 (1H, d, J7.2), 4.72 (1H, s), 4.65 (1H, s), 4.09-3.88 (6H, m), 3.24-3.15 (2H, m), 2.61 (2H, d, J 12.1), 2.44 (2H, d, J 13.8), 2.03-1.88 (2H, m), 1.87-1.78 (2H, m), 1.66 (11H, s), 1.61-1.40 (4H, m), 1.07 (3H, d, J 12.9); UPLC (Method A): 2.30 min, 99%, MS (ESI) 358.2 (M+H)⁺.

### Example 129: Synthesis of 1-(2-(3,8-diazabicyclo[3.2.1]octan-8-yl)-6,7-dihydrothiazolo[5,4-c]pyridin-5(4H)-yl)-2-(2-(2-methoxyethoxy)phenoxy)ethan-1-one

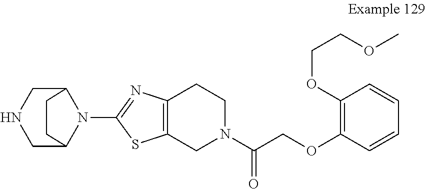 [SMILES: COCCOc1ccccc1OCC(=O)N1CCc2nc(N3C4CCC3CNC4)sc2C1]

Prepared according to the method of Example 73 using 2-(2-methoxyethoxy)phenol as the starting reagent.

¹H-NMR: δ_{H} (400 MHz, CDCl₃, mixture of rotamers) 7.06-6.78 (4H, m), 4.80 (2H, d, J 7.3), 4.70 (0.8H, s), 4.66-4.59 (1.2H, m), 4.22-4.14 (2H, m), 4.14-4.06 (2H, m), 3.95-3.85 (2H, m), 3.82-3.72 (2H, m), 3.48-3.39 (3H, m), 3.22 (2H, d, J 12.2), 2.81-2.70 (2H, m), 2.70-2.62 (2H, m), 2.13-1.98 (2H, m), 1.98-1.87 (2H, m); UPLC (Method A): t_{R} 1.48 min, 97%, MS (ESI) 459.2 (M+H)⁺.

The following examples (130-243) were prepared using procedures analogous to those used in Example 73, steps 3 and 4, starting from Intermediate 9 and the appropriate carboxylic acid starting materials.

### Example 130: Synthesis of 1-(2-(3,8-diazabicyclo[3.2.1]octan-8-yl)-6,7-dihydrothiazolo[5,4-c]pyridin-5(4H)-yl)-2-(1-methylcyclobutyl)ethan-1-one

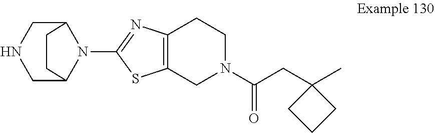 [SMILES: CC1(CC(=O)N2CCc3nc(N4C5CCC4CNC5)sc3C2)CCC1]

Prepared using 2-(1-methylcyclobutyl)acetic acid as the acid component.

¹H-NMR: δ_{H} (400 MHz, CDCl₃, mixture of rotamers) 4.70-4.47 (2H, m), 4.24-4.08 (2H, m), 3.97-3.70 (2H, m), 3.22 (2H, d, J 12.3), 2.80-2.63 (4H, m), 2.49 (2H, d, J 15.9), 2.17-1.74 (11H, m), 1.35-1.21 (3H, m); UPLC (Method A): t_{R} 1.55 min, 100%, MS (ESI) 361.2 (M+H)⁺.

### Example 131: Synthesis of 1-(2-(3,8-diazabicyclo[3.2.1]octan-8-yl)-6,7-dihydrothiazolo[5,4-c]pyridin-5(4H)-yl)-2-(4-fluorophenyl)-2-methoxyethan-1-one

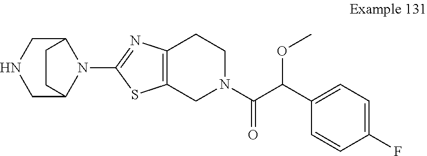 [SMILES: COC(C(=O)N1CCc2nc(N3C4CCC3CNC4)sc2C1)c1ccc(F)cc1]

Prepared using 2-(4-fluorophenyl)-2-methoxyacetic acid as the acid component.

¹H-NMR: δ_{H} (400 MHz, CDCl₃, mixture of rotamers) 7.63-7.32 (2H, m), 7.26-6.94 (2H, m), 5.22-5.02 (1H, m), 4.79-4.37 (2H, m), 4.25-4.05 (2H, m), 4.04-3.44 (5H, m), 3.30-3.14 (2H, m), 2.87-2.28 (4H, m), 2.16-1.87 (4H, m); UPLC (Method A): t_{R} 1.42 min, 98%, MS (ESI) 417.2 (M+H)⁺.

### Example 132: Synthesis of 1-(2-(3,8-diazabicyclo[3.2.1]octan-8-yl)-6,7-dihydrothiazolo[5,4-c]pyridin-5(4H)-yl)-2-(4-fluorophenyl)-2-hydroxyethan-1-one

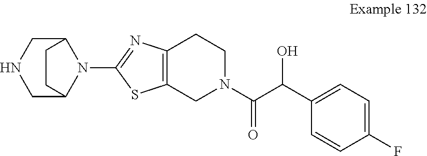 [SMILES: O=C(C(O)c1ccc(F)cc1)N1CCc2nc(N3C4CCC3CNC4)sc2C1]

Prepared using 2-(4-fluorophenyl)-2-hydroxyacetic acid as the acid component.

¹H-NMR: δ_{H} (400 MHz, CDCl₃, mixture of rotamers) 7.40-7.31 (2H, m), 7.13-6.99 (2H, m), 5.37-5.21 (1H, m), 4.94-4.83 (0.7H, m), 4.59-4.48 (0.7H, m), 4.39-4.31 (0.3H, m), 4.15-4.06 (2H, m), 4.02-3.95 (0.3H, m), 3.90-3.41 (2H, m), 3.26-3.07 (2H, m), 2.81-2.37 (3.5H, m), 2.16-1.88 (5.5H, m); UPLC (Method A): t_{R} 1.31 min, 100%, MS (ESI) 403.2 (M+H)⁺.

### Example 133: Synthesis of (2-(3,8-diazabicyclo[3.2.1]octan-8-yl)-6,7-dihydrothiazolo[5,4-c]pyridin-5(4H)-yl)(tetrahydrofuran-2-yl)methanone

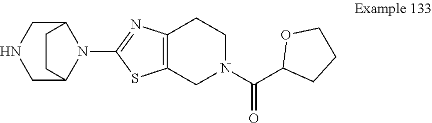 [SMILES: O=C(C1CCCO1)N1CCc2nc(N3C4CCC3CNC4)sc2C1]

Prepared using tetrahydrofuran-2-carboxylic acid as the acid component.

¹H-NMR: δ_{H} (400 MHz, CDCl₃, mixture of rotamers) 4.74-4.55 (3H, m), 4.17 (2H, s), 4.12-3.65 (4H, m), 3.26 (2H, d, J 12.1), 2.84-2.65 (4H, m), 2.39-2.25 (1H, m), 2.17-1.85 (7H, m); UPLC (Method A): t_{R} 1.00 min, 100%, MS (ESI) 349.2 (M+H)⁺.

### Example 134: Synthesis of 1-(2-(3,8-diazabicyclo[3.2.1]octan-8-yl)-6,7-dihydrothiazolo[5,4-c]pyridin-5(4H)-yl)-2-(1-methoxycyclopentyl)ethan-1-one

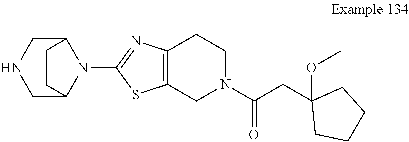 [SMILES: COC1(CC(=O)N2CCc3nc(N4C5CCC4CNC5)sc3C2)CCCC1]

Prepared using 2-(1-methoxycyclopentyl)acetic acid as the acid component.

¹H-NMR: δ_{H} (400 MHz, CDCl₃, mixture of rotamers) 4.67-4.52 (2H, m), 4.12 (2H, d, J 4.2), 3.95-3.75 (2H, m), 3.27-3.09 (5H, m), 2.80-2.59 (6H, m), 2.12-1.98 (2H, m), 1.97-1.81 (4H, m), 1.77-1.64 (6H, m); UPLC (Method A): t_{R} 1.37 min, 97%, MS (ESI) 391.2 (M+H)⁺.

### Example 135: Synthesis of 1-(2-(3,8-diazabicyclo[3.2.1]octan-8-yl)-6,7-dihydrothiazolo[5,4-c]pyridin-5(4H)-yl)-2-(1-(methoxymethyl)cyclopentyl)ethan-1-one

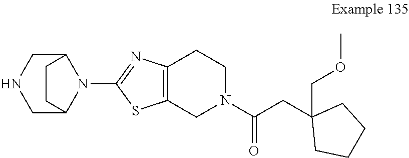 [SMILES: COCC1(CC(=O)N2CCc3nc(N4C5CCC4CNC5)sc3C2)CCCC1]

Prepared using 2-(1-(methoxymethyl)cyclopentyl)acetic acid as the acid component.

¹H-NMR: δ_{H} (400 MHz, CDCl₃, mixture of rotamers) 4.65-4.55 (2H, m), 4.18 (2H, d, J 4.8), 3.97-3.75 (2H, m), 3.54 (1H, s), 3.36-3.16 (7H, m), 2.79 (2H, d, J 12.1), 2.75-2.63 (2H, m), 2.57-2.45 (2H, m), 2.19-1.95 (4H, m), 1.70-1.50 (8H, m); UPLC (Method A): t_{R} 1.61 min, 100%, MS (ESI) 405.2 (M+H)⁺.

### Example 136: Synthesis of 1-(2-(3,8-diazabicyclo[3.2.1]octan-8-yl)-6,7-dihydrothiazolo[5,4-c]pyridin-5(4H)-yl)-2-(pyrazin-2-yl)ethan-1-one

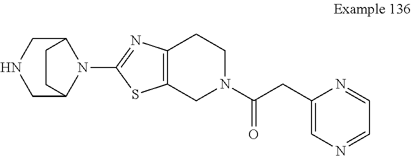 [SMILES: O=C(Cc1cnccn1)N1CCc2nc(N3C4CCC3CNC4)sc2C1]

Prepared using 2-pyrazineacetic acid as the acid component.

¹H-NMR: δ_{H} (400 MHz, CDCl₃, mixture of rotamers) 8.67-8.62 (1H, m), 8.53-8.45 (2H, m), 4.69-4.59 (2H, m), 4.17-4.08 (2H, m), 4.04 (1.3H, s), 4.00 (0.7H, s), 3.96-3.85 (2H, m), 3.26-3.18 (2H, m), 2.74-2.61 (4H, m), 2.13-2.01 (2H, m), 1.98-1.88 (2H, m); UPLC (Method A): t_{R} 1.15 min, 99%, MS (ESI) 371.2 (M+H)⁺.

### Example 137: Synthesis of 1-(2-(3,8-diazabicyclo[3.2.1]octan-8-yl)-6,7-dihydrothiazolo[5,4-c]pyridin-5(4H)-yl)-2-(3,3-difluorocyclopentyl)ethan-1-one

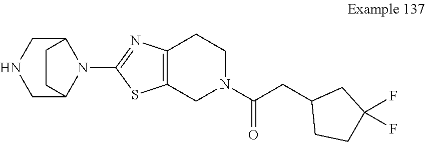 [SMILES: O=C(CC1CCC(F)(F)C1)N1CCc2nc(N3C4CCC3CNC4)sc2C1]

Prepared using 2-(3,3-difluorocyclopentyl)acetic acid as the acid component.

¹H-NMR: δ_{H} (400 MHz, CDCl₃, mixture of rotamers) 4.62 (1.3H, s), 4.49 (0.7H, s), 4.21 (2H, d, J 4.9), 3.89 (0.7H, t, J5.9), 3.73 (1.3H, t, J5.8), 3.37-3.28 (2H, m), 2.87 (2H, d, J12.3), 2.77-2.56 (3H, m), 2.56-2.35 (3H, m), 2.25-1.98 (7H, m), 1.84-1.66 (1H, m), 1.54-1.39 (1H, m); UPLC (Method A): t_{R} 2.12 min, 99%, MS (ESI) 397.2 (M+H)⁺.

### Example 138: Synthesis of 1-(2-(3,8-diazabicyclo[3.2.1]octan-8-yl)-6,7-dihydrothiazolo[5,4-c]pyridin-5(4H)-yl)-2-(2-methoxyphenoxy)ethan-1-one

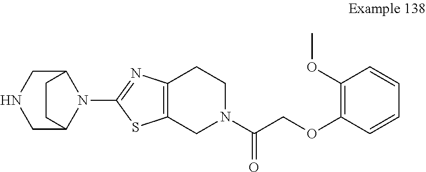 [SMILES: COc1ccccc1OCC(=O)N1CCc2nc(N3C4CCC3CNC4)sc2C1]

Prepared using 2-(2-methoxyphenoxy)acetic acid as the acid component.

¹H-NMR: δ_{H} (400 MHz, CDCl₃, mixture of rotamers) 7.01-6.94 (2H, m), 6.93-6.85 (2H, m), 4.85-4.79 (2H, m), 4.75 (1H, s), 4.71-4.58 (2H, m), 4.19-4.08 (2H, m), 3.93-3.86 (5H, m), 3.21 (2H, d, J 12.3), 2.79-2.61 (4H, m), 2.10-2.02 (2H, m), 1.97-1.89 (2H, m); UPLC (Method A): t_{R} 1.45 min, 99%, MS (ESI) 415.2 (M+H)⁺.

### Example 139: Synthesis of 1-(2-(3,8-diazabicyclo[3.2.1]octan-8-yl)-6,7-dihydrothiazolo[5,4-c]pyridin-5(4H)-yl)-2-(2-methoxyphenyl)ethan-1-one

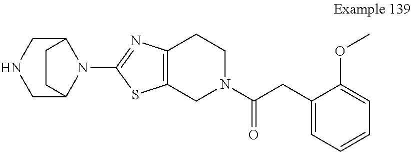 [SMILES: COc1ccccc1CC(=O)N1CCc2nc(N3C4CCC3CNC4)sc2C1]

Prepared using 2-(2-methoxyphenyl)acetic acid as the acid component.

¹H-NMR: δ_{H} (400 MHz, CDCl₃, mixture of rotamers) 7.26-7.17 (2H, m), 6.95-6.83 (2H, m), 4.66-4.48 (2H, m), 4.14-4.08 (2H, m), 3.92 (0.6H, t, J 5.8), 3.84 (2H, s), 3.82-3.78 (2.4H, m), 3.76-3.71 (2H, m), 3.21 (2H, d, J 12.3), 2.74-2.48 (4H, m), 2.08-1.89 (4H, m); UPLC (Method A): t_{R} 1.47 min, 100%, MS (ESI) 399.2 (M+H)⁺.

### Example 140: Synthesis of 1-(2-(3,8-diazabicyclo[3.2.1]octan-8-yl)-6,7-dihydrothiazolo[5,4-c]pyridin-5(4H)-yl)-2-(4-fluoro-2-methoxyphenyl)ethan-1-one

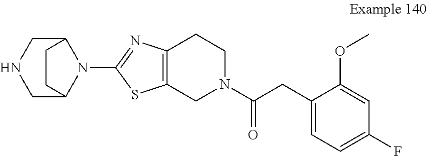 [SMILES: COc1cc(F)ccc1CC(=O)N1CCc2nc(N3C4CCC3CNC4)sc2C1]

Prepared using 2-(4-fluoro-2-methoxyphenyl)acetic acid as the acid component.

¹H-NMR: δ_{H} (400 MHz, CDCl₃, mixture of rotamers) 7.21-7.09 (1H, m), 6.67-6.56 (2H, m), 4.69-4.46 (2H, m), 4.17-4.07 (2H, m), 3.91 (1H, t, J 5.8), 3.82 (2H, s), 3.78 (1H, s), 3.77-3.66 (3.4H, m), 3.21 (2H, d, J 12.2), 2.74-2.53 (4H, m), 2.12-1.88 (4H, m); UPLC (Method A): t_{R} 1.53 min, 97%, MS (ESI) 417.2 (M+H)⁺.

### Example 141: Synthesis of 1-(2-(3,8-diazabicyclo[3.2.1]octan-8-yl)-6,7-dihydrothiazolo[5,4-c]pyridin-5(4H)-yl)-2-(2-fluoropyridin-4-yl)ethan-1-one

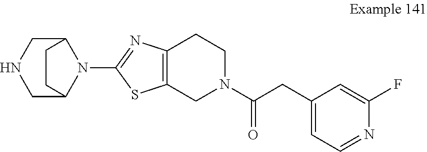 [SMILES: O=C(Cc1ccnc(F)c1)N1CCc2nc(N3C4CCC3CNC4)sc2C1]

Prepared using 2-fluoropyridine-4-acetic acid as the acid component.

¹H-NMR: δ_{H} (400 MHz, CDCl₃, mixture of rotamers) 8.21-8.12 (1H, m), 7.11 (0.6H, d, J 5.1), 7.07 (0.4H, d, J 5.1), 6.87 (0.6H, s), 6.82 (0.4H, s), 4.65 (1.3H, d, J 2.2), 4.47 (0.7H, d, J 2.2), 4.18-4.07 (2H, m), 3.93 (0.7H, t, J 5.8), 3.86-3.77 (2H, m), 3.73 (1.3H, t, J 5.8), 3.27-3.17 (2H, m), 2.77-2.62 (4H, m), 2.12-1.91 (4H, m); UPLC (Method A): t_{R} 1.51 min, 97%, MS (ESI) 388.2 (M+H)⁺.

### Example 142: Synthesis of 1-(2-(3,8-diazabicyclo[3.2.1]octan-8-yl)-6,7-dihydrothiazolo[5,4-c]pyridin-5(4H)-yl)-3-(6-fluoropyridin-3-yl)propan-1-one

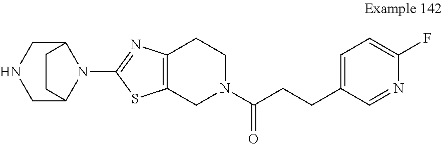 [SMILES: O=C(CCc1ccc(F)nc1)N1CCc2nc(N3C4CCC3CNC4)sc2C1]

Prepared using 3-(6-fluoropyridin-3-yl)propanoic acid as the acid component.

¹H-NMR: δ_{H} (400 MHz, CDCl₃, mixture of rotamers) 8.12-8.04 (1H, m), 7.73-7.63 (1H, m), 6.90-6.79 (1H, m), 4.68-4.38 (2H, m), 4.18-4.06 (2H, m), 3.95-3.64 (2H, m), 3.21 (2H, dd, J 12.4, 1.7), 3.08-2.95 (2H, m), 2.76-2.61 (6H, m), 2.13-1.89 (4H, m); UPLC (Method A): t_{R} 1.24 min, 100%, MS (ESI) 402.2 (M+H)⁺.

### Example 143: Synthesis of 1-(2-(3,8-diazabicyclo[3.2.1]octan-8-yl)-6,7-dihydrothiazolo[5,4-c]pyridin-5(4H)-yl)-2-(4-fluoro-2-methoxyphenoxy)ethan-1-one

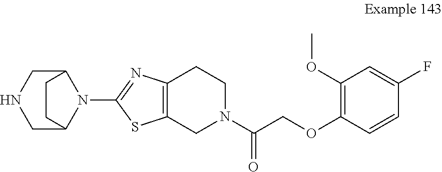 [SMILES: COc1cc(F)ccc1OCC(=O)N1CCc2nc(N3C4CCC3CNC4)sc2C1]

Prepared using 2-(4-fluoro-2-methoxyphenoxy)acetic acid as the acid component.

¹H-NMR: δ_{H} (400 MHz, CDCl₃, mixture of rotamers) 6.96-6.87 (1H, m), 6.69-6.61 (1H, m), 6.61-6.52 (1H, m), 4.76 (2H, d, J 11.0), 4.67 (0.7H, s), 4.65-4.59 (1.3H, m), 4.12 (2H, s), 3.91-3.86 (2H, m), 3.85 (3H, s), 3.22 (2H, d, J 12.3), 2.80-2.61 (4H, m), 2.12-1.98 (2H, m), 1.98-1.89 (2H, m); UPLC (Method A): t_{R} 2.28 min, 99%, MS (ESI) 433.2 (M+H)⁺.

### Example 144: Synthesis of 1-(2-(3,8-diazabicyclo[3.2.1]octan-8-yl)-6,7-dihydrothiazolo[5,4-c]pyridin-5(4H)-yl)-2-(2,4-difluorophenyl)ethan-1-one

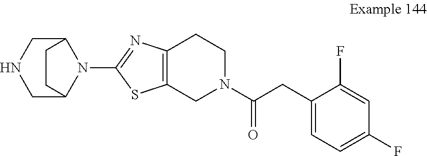 [SMILES: O=C(Cc1ccc(F)cc1F)N1CCc2nc(N3C4CCC3CNC4)sc2C1]

Prepared using 2,4-difluorophenylacetic acid as the acid component.

¹H-NMR: δ_{H} (400 MHz, CDCl₃, mixture of rotamers) 7.37-7.24 (1H, m), 7.24-7.13 (1H, m), 7.08-6.96 (1H, m), 4.64 (0.7H, s), 4.51 (1.3H, s), 4.04 (2H, s), 3.90-3.71 (4H, m), 2.98 (2H, d, J 12.4), 2.72-2.58 (3H, m), 2.53 (1H, s), 1.90 (4H, s); UPLC (Method A): t_{R} 2.21 min, 99%, MS (ESI) 405.2 (M+H)⁺.

### Example 145: Synthesis of 1-(2-(3,8-diazabicyclo[3.2.1]octan-8-yl)-6,7-dihydrothiazolo[5,4-c]pyridin-5(4H)-yl)-2-(4-fluoro-2-methylphenoxy)ethan-1-one

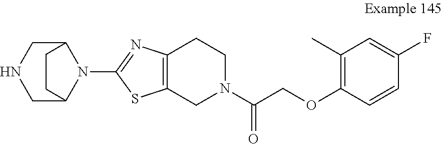 [SMILES: Cc1cc(F)ccc1OCC(=O)N1CCc2nc(N3C4CCC3CNC4)sc2C1]

Prepared using 2-(4-fluoro-2-methylphenoxy)acetic acid as the acid component.

¹H-NMR: δ_{H} (400 MHz, CDCl₃, mixture of rotamers) 6.90-6.83 (1H, m), 6.83-6.74 (2H, m), 4.73 (2H, d, J 14.1), 4.67-4.58 (2H, m), 4.12 (2H, s), 3.96-3.82 (2H, m), 3.21 (2H, d, J 12.2), 2.72 (2H, d, J 5.5), 2.70-2.62 (2H, m), 2.25 (3H, d, J 9.5), 2.11-2.00 (2H, m), 2.00-1.89 (2H, m); UPLC (Method A): t_{R} 2.63 min, 99%, MS (ESI) 417.2 (M+H)⁺.

### Example 146: Synthesis of 1-(2-(3,8-diazabicyclo[3.2.1]octan-8-yl)-6,7-dihydrothiazolo[5,4-c]pyridin-5(4H)-yl)-2-cyclopentyl-2-methylpropan-1-one

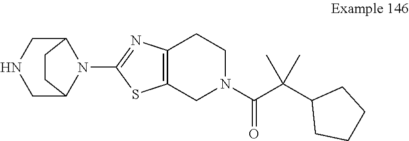 [SMILES: CC(C)(C(=O)N1CCc2nc(N3C4CCC3CNC4)sc2C1)C1CCCC1]

Prepared using 2-cyclopentyl-2-methylpropanoic acid as the acid component.

¹H-NMR: δ_{H} (400 MHz, CDCl₃, mixture of rotamers) 4.61 (2H, d, J2.4), 4.13 (2H, t, J3.6), 3.91 (2H, t, J5.7), 3.22 (2H, d, J 12.3), 2.71 (2H, t, J 5.8), 2.68-2.63 (2H, m), 2.39-2.25 (1H, m), 2.12-2.00 (2H, m), 1.97-1.88 (2H, m), 1.61-1.43 (6H, m), 1.38-1.27 (2H, m), 1.25 (6H, s); UPLC (Method A): t_{R} 2.94 min, 100%, MS (ESI) 389.2 (M+H)⁺.

### Example 147: Synthesis of 1-(2-(3,8-diazabicyclo[3.2.1]octan-8-yl)-6,7-dihydrothiazolo[5,4-c]pyridin-5(4H)-yl)-3-(4-fluoro-2-methoxyphenyl)propan-1-one

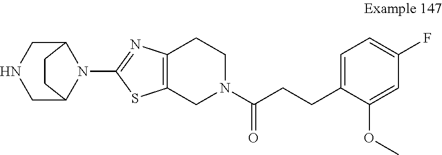 [SMILES: COc1cc(F)ccc1CCC(=O)N1CCc2nc(N3C4CCC3CNC4)sc2C1]

Prepared using 3-(4-fluoro-2-methoxyphenyl)propanoic acid as the acid component.

¹H-NMR: δ_{H} (400 MHz, CDCl₃, mixture of rotamers) 7.15-7.04 (1H, m), 6.63-6.49 (2H, m), 4.62 (1.3H, d, J2.0), 4.44 (0.7H, d, J2.2), 4.18-4.05 (2H, m), 3.89 (0.7H, t, J5.8), 3.81 (3H, s), 3.71 (1.3H, t, J 5.8), 3.22 (2H, d, J 12.2), 2.98-2.84 (2H, m), 2.73-2.54 (6H, m), 2.11-2.01 (2H, m), 1.97-1.89 (2H, m); UPLC (Method A): t_{R} 2.51 min, 99%, MS (ESI) 431.2 (M+H)⁺.

### Example 148: Synthesis of 1-(2-(3,8-diazabicyclo[3.2.1]octan-8-yl)-6,7-dihydrothiazolo[5,4-c]pyridin-5(4H)-yl)-2-(2,4-difluorophenoxy)ethan-1-one

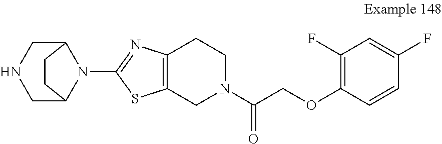 [SMILES: O=C(COc1ccc(F)cc1F)N1CCc2nc(N3C4CCC3CNC4)sc2C1]

Prepared using 2-(2,4-difluorophenoxy)acetic acid as the acid component.

¹H-NMR: δ_{H} (400 MHz, CDCl₃, mixture of rotamers) 7.09-6.97 (1H, m), 6.91-6.82 (1H, m), 6.82-6.74 (1H, m), 4.80 (2H, d, J9.2), 4.68-4.57 (2H, m), 4.13 (2H, s), 3.94-3.81 (2H, m), 3.23 (2H, d, J 12.3), 2.82-2.73 (1.3H, m), 2.73-2.65 (2.7H, m), 2.16-1.91 (4H, m); UPLC (Method A): t_{R} 2.29 min, 100%, MS (ESI) 421.2 (M+H)⁺.

### Example 149: Synthesis of 1-(2-(3,8-diazabicyclo[3.2.1]octan-8-yl)-6,7-dihydrothiazolo[5,4-c]pyridin-5(4H)-yl)-2-(cyclopentyloxy)ethan-1-one

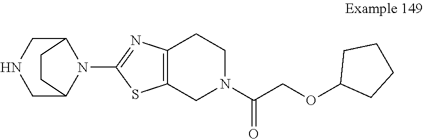 [SMILES: O=C(COC1CCCC1)N1CCc2nc(N3C4CCC3CNC4)sc2C1]

Prepared using 2-(cyclopentyloxy)acetic acid as the acid component.

¹H-NMR: δ_{H} (400 MHz, CDCl₃, mixture of rotamers) 4.64-4.55 (2H, m), 4.23-4.09 (4H, m), 4.04-3.93 (1H, m), 3.93-3.84 (0.7H, m), 3.84-3.74 (1.3H, m), 3.28-3.16 (2H, m), 2.81-2.73 (1.3H, m), 2.73-2.65 (2.7H, m), 2.14-1.93 (4H, m), 1.75-1.61 (6H, m), 1.61-1.45 (2H, m); UPLC (Method A): t_{R} 2.01 min, 97%, MS (ESI) 377.2 (M+H)⁺.

### Example 150: Synthesis of (2-(3,8-diazabicyclo[3.2.1]octan-8-yl)-6,7-dihydrothiazolo[5,4-c]pyridin-5(4H)-yl)(1-methylcyclopentyl)methanone

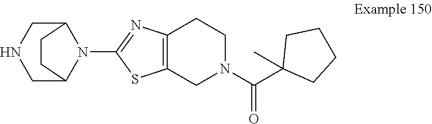 [SMILES: CC1(C(=O)N2CCc3nc(N4C5CCC4CNC5)sc3C2)CCCC1]

Prepared using 1-methylcyclopentane-1-carboxylic acid as the acid component.

¹H-NMR: δ_{H} (400 MHz, CDCl₃, mixture of rotamers) 4.62-4.51 (2H, m), 4.18-4.09 (2H, m), 3.90-3.79 (2H, m), 3.30-3.18 (2H, m), 2.75-2.65 (4H, m), 2.31-2.14 (2H, m), 2.14-2.02 (2H, m), 1.98-1.90 (2H, m), 1.67-1.56 (6H, m), 1.31 (3H, s); UPLC (Method A): t_{R} 1.58 min, 99%, MS (ESI) 361.4 (M+H)⁺.

### Example 151: Synthesis of (2-(3,8-diazabicyclo[3.2.1]octan-8-yl)-6,7-dihydrothiazolo[5,4-c]pyridin-5(4H)-yl)(1-fluorocyclopentyl)methanone

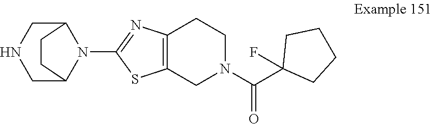 [SMILES: O=C(N1CCc2nc(N3C4CCC3CNC4)sc2C1)C1(F)CCCC1]

Prepared using 1-fluorocyclopentane-1-carboxylic acid as the acid component.

¹H-NMR: δ_{H} (400 MHz, CDCl₃, mixture of rotamers) 4.71 (0.7H, s), 4.61 (1.3H, s), 4.19-4.06 (2H, m), 4.03-3.78 (2H, m), 3.28-3.15 (2H, m), 2.75 (2H, s), 2.70-2.62 (2H, m), 2.47-2.21 (2H, m), 2.21-1.97 (4H, m), 1.98-1.89 (2H, m), 1.89-1.69 (4H, m); UPLC (Method A): t_{R} 1.56 min, 100%, MS (ESI) 365.4 (M+H)⁺.

### Example 152: Synthesis of 3-(2-(3,8-diazabicyclo[3.2.1]octan-8-yl)-6,7-dihydrothiazolo[5,4-c]pyridin-5(4H)-yl)-2-cyclopentyl-3-oxopropanenitrile

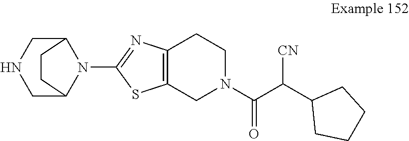 [SMILES: N#CC(C(=O)N1CCc2nc(N3C4CCC3CNC4)sc2C1)C1CCCC1]

Prepared using 2-cyano-2-cyclopentylacetic acid as the acid component.

¹H-NMR: δ_{H} (400 MHz, CDCl₃, mixture of rotamers) 4.70-4.46 (2H, m), 4.13 (2H, s), 4.10-4.02 (0.35H, m), 3.85-3.80 (1H, m), 3.80-3.71 (0.65H, m), 3.68-3.58 (1H, m), 3.29-3.15 (2H, m), 2.92-2.70 (2H, m), 2.69-2.62 (2H, m), 2.60-2.39 (1H, m), 2.13-1.81 (6H, m), 1.78-1.61 (4H, m), 1.58-1.46 (2H, m), 1.35-1.23 (1H, m); UPLC (Method A): t_{R} 2.19 min, 97%, MS (ESI) 386.2 (M+H)⁺.

### Example 153: Synthesis of 1-(2-(3,8-diazabicyclo[3.2.1]octan-8-yl)-6,7-dihydrothiazolo[5,4-c]pyridin-5(4H)-yl)-2-(2,6-dimethylpyridin-4-yl)ethan-1-one

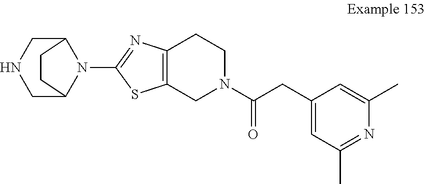 [SMILES: Cc1cc(CC(=O)N2CCc3nc(N4C5CCC4CNC5)sc3C2)cc(C)n1]

Prepared using 2-(2,6-dimethylpyridin-4-yl)acetic acid as the acid component.

¹H-NMR: δ_{H} (400 MHz, CDCl₃, mixture of rotamers) 7.01-6.94 (1H, m), 6.88-6.82 (1H, m), 4.70-4.59 (2H, m), 4.22-4.13 (2H, m), 3.97-3.85 (4H, m), 3.31-3.25 (2H, m), 2.85-2.77 (2H, m), 2.71-2.51 (2H, m), 2.50-2.44 (3H, m), 2.28-2.24 (3H, m), 2.18-1.97 (4H, m); UPLC (Method A): t_{R} 1.07 min, 100%, MS (ESI) 398.2 (M+H)⁺.

### Example 154: Synthesis of 1-(2-(3,8-diazabicyclo[3.2.1]octan-8-yl)-6,7-dihydrothiazolo[5,4-c]pyridin-5(4H)-yl)-2,2-dimethylbutan-1-one

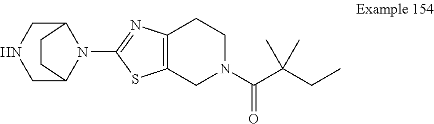 [SMILES: CCC(C)(C)C(=O)N1CCc2nc(N3C4CCC3CNC4)sc2C1]

Prepared using 2,2-dimethylbutyric acid as the acid component.

¹H-NMR: δ_{H} (400 MHz, CDCl₃) 4.61 (2H, t, J2.0), 4.17-4.10 (2H, m), 3.89 (2H, t, J5.7), 3.24 (2H, dd, J 12.2, 1.8), 2.75-2.64 (4H, m), 2.10-1.90 (4H, m), 1.67 (3H, t, J 7.4), 1.28 (6H, s), 0.88 (3H, t, J 7.5); UPLC (Method A): t_{R} 1.41 min, 99%, MS (ESI) 349.2 (M+H)⁺.

### Example 155: Synthesis of 1-(2-(3,8-diazabicyclo[3.2.1]octan-8-yl)-6,7-dihydrothiazolo[5,4-c]pyridin-5(4H)-yl)-2-(2-oxaspiro[3.5]nonan-7-yl)ethan-1-one

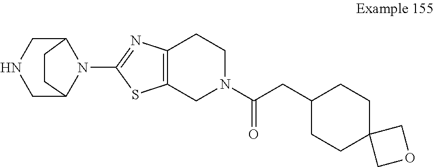 [SMILES: O=C(CC1CCC2(CC1)COC2)N1CCc2nc(N3C4CCC3CNC4)sc2C1]

Prepared using 2-(2-oxaspiro[3.5]nonan-7-yl)acetic acid as the acid component.

¹H-NMR: δ_{H} (400 MHz, CDCl₃, mixture of rotamers) 4.67-4.55 (1.3H, m), 4.47 (0.7H, t, J2.0), 4.44-4.37 (2H, m), 4.37-4.29 (2H, m), 4.20-4.05 (2H, m), 3.89 (0.7H, t, J5.8), 3.72 (1.3H, t, J 5.8), 3.22 (2H, d, J 12.2), 2.77-2.62 (4H, m), 2.32-2.19 (2H, m), 2.17-2.08 (2H, m), 2.08-1.98 (2H, m), 1.98-1.89 (2H, m), 1.88-1.68 (3H, m), 1.52-1.39 (2H, m), 1.02-0.85 (2H, m); UPLC (Method A): t_{R} 1.33 min, 99%, MS (ESI) 417.2 (M+H)⁺.

### Example 156: Synthesis of 1-(2-(3,8-diazabicyclo[3.2.1]octan-8-yl)-6,7-dihydrothiazolo[5,4-c]pyridin-5(4H)-yl)-2-(4-fluoro-2-methylphenyl)ethan-1-one

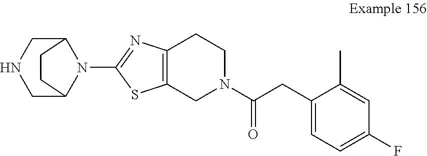 [SMILES: Cc1cc(F)ccc1CC(=O)N1CCc2nc(N3C4CCC3CNC4)sc2C1]

Prepared using 4-fluoro-2-methylphenylacetic acid as the acid component.

¹H-NMR: δ_{H} (400 MHz, CDCl₃, mixture of rotamers) 7.11-6.99 (1H, m), 6.92-6.78 (2H, m), 4.66 (1.3H, br s), 4.46 (0.7H, br s), 4.17-4.09 (2H, m), 3.94 (0.7H, t, J5.8), 3.75-3.66 (3.3H, m), 3.23 (2H, dd, J 12.4, 1.9), 2.75-2.57 (4H, m), 2.28 (2H, s), 2.26 (1H, s), 2.12-2.02 (2H, m), 1.99-1.91 (2H, m); UPLC (Method A): t_{R} 1.54 min, 99%, MS (ESI) 401.2 (M+H)⁺.

### Example 157: Synthesis of 1-(2-(3,8-diazabicyclo[3.2.1]octan-8-yl)-6,7-dihydrothiazolo[5,4-c]pyridin-5(4H)-yl)-2-cyclopentyl-2-methoxyethan-1-one

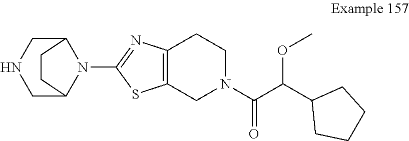 [SMILES: COC(C(=O)N1CCc2nc(N3C4CCC3CNC4)sc2C1)C1CCCC1]

Prepared using 2-cyclopentyl-2-methoxyacetic acid as the acid component.

¹H-NMR: δ_{H} (400 MHz, CDCl₃, mixture of rotamers) 4.84-4.75 (0.7H, m), 4.74-4.54 (1.3H, m), 4.21-4.03 (2.6H, m), 4.03-3.74 (2.4H, m), 3.41-3.29 (3H, m), 3.29-3.18 (2H, m), 2.82-2.63 (4H, m), 2.34-1.80 (5H, m), 1.70-1.20 (8H, m); UPLC (Method A): t_{R} 1.46 min, 96%, MS (ESI) 391.2 (M+H)⁺.

### Examples 158 and 159: Synthesis of both enantiomers of 1-(2-(3,8-diazabicyclo[3.2.1]octan-8-yl)-6,7-dihydrothiazolo[5,4-c]pyridin-5(4H)-yl)-2,3,3-trimethylbutan-1-one

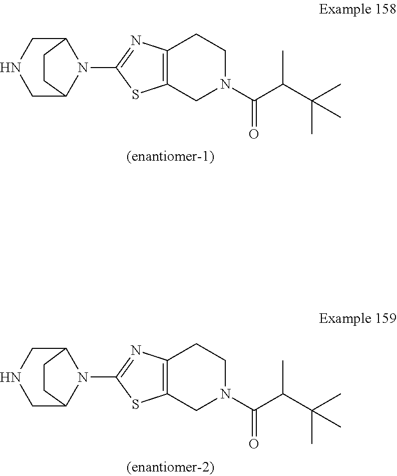 [SMILES: CC(C(=O)N1CCc2nc(N3C4CCC3CNC4)sc2C1)C(C)(C)C ; CC(C(=O)N1CCc2nc(N3C4CCC3CNC4)sc2C1)C(C)(C)C]

Prepared using 2,3,3-trimethylbutanoic acid as the acid component. The enantiomers were separated by chiral preparative SFC (Phenomenex Lux Amylose-1 column, CO₂/20 mM NH₃ in MeOH eluent).

First eluting isomer (Example 158): ¹H-NMR: δ_{H} (400 MHz, CDCl₃, mixture of rotamers) 4.81-4.70 (0.7H, m), 4.69-4.43 (1.3H, m), 4.20-4.07 (2H, m), 4.02-3.88 (1H, m), 3.88-3.80 (0.3H, m), 3.80-3.70 (0.7H, m), 3.23 (2H, d, J 12.2), 2.80-2.58 (5H, m), 2.12-1.98 (2H, m), 1.98-1.87 (2H, m), 1.18-1.05 (3H, m), 1.05-0.91 (9H, m); UPLC (Method A): t_{R} 1.55 min, 100%, MS (ESI) 363.2 (M+H)⁺; SFC (Phenomenex Lux Amylose-1 column, CO₂/NH₃ in MeOH eluent): t_{R} 3.58 min, 100%.

Second eluting isomer (Example 159): ¹H-NMR: δ_{H} (400 MHz, CDCl₃, mixture of rotamers) 4.80-4.69 (0.7H, m), 4.69-4.44 (1.3H, m), 4.19-4.06 (2H, m), 4.02-3.88 (1H, m), 3.88-3.80 (0.3H, m), 3.80-3.70 (0.7H, m), 3.23 (2H, d, J 12.2), 2.80-2.59 (5H, m), 2.12-1.98 (2H, m), 1.98-1.87 (2H, m), 1.17-1.06 (3H, m), 1.06-0.91 (9H, m); UPLC (Method A): t_{R} 1.55 min, 100%, MS (ESI) 363.2 (M+H)⁺; SFC (Phenomenex Lux Amylose-1 column, CO₂/NH₃ in MeOH eluent): t_{R} 3.94 min, 99%.

### Example 160: Synthesis of 1-(2-(3,8-diazabicyclo[3.2.1]octan-8-yl)-6,7-dihydrothiazolo[5,4-c]pyridin-5(4H)-yl)-2-(bicyclo[2.2.1]heptan-1-yl)ethan-1-one

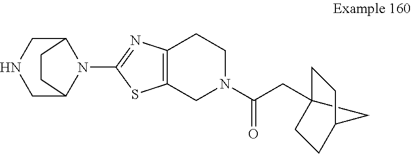 [SMILES: O=C(CC12CCC(CC1)C2)N1CCc2nc(N3C4CCC3CNC4)sc2C1]

Prepared using 2-{bicyclo[2.2.1]heptan-1-yl}acetic acid as the acid component.

¹H-NMR: δ_{H} (400 MHz, CDCl₃, mixture of rotamers) 4.65-4.60 (1.3H, m), 4.55-4.50 (0.7H, m), 4.18-4.09 (2H, m), 3.90 (0.7H, t, J 5.8), 3.77 (1.3H, t, J 5.8), 3.23 (2H, dt, J 12.3, 2.0), 2.75-2.63 (6H, m), 2.22-2.15 (1H, m), 2.10-1.99 (2H, m), 1.99-1.91 (2H, m), 1.66-1.55 (3H, m), 1.55-1.36 (4H, m), 1.36-1.23 (4H, m); UPLC (Method A): t_{R} 1.65 min, 98%, MS (ESI) 387.2 (M+H)⁺.

### Example 161: Synthesis of 1-(2-(3,8-diazabicyclo[3.2.1]octan-8-yl)-6,7-dihydrothiazolo[5,4-c]pyridin-5(4H)-yl)-2,2,3-trimethylbutan-1-one

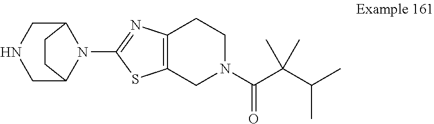 [SMILES: CC(C)C(C)(C)C(=O)N1CCc2nc(N3C4CCC3CNC4)sc2C1]

Prepared using 2,2,3-trimethylbutanoic acid as the acid component.

¹H-NMR: δ_{H} (400 MHz, CDCl₃) 4.62 (2H, t, J2.1), 4.14 (2H, br s), 3.91 (2H, t, J 5.7), 3.24 (2H, dd, J 12.5, 1.9), 2.74-2.65 (4H, m), 2.18-2.04 (3H, m), 2.01-1.92 (2H, m), 1.21 (6H, s), 0.88 (6H, d, J 6.7); UPLC (Method A): t_{R} 1.54 min, 96%, MS (ESI) 363.2 (M+H)⁺.

### Example 162: Synthesis of (2-(3,8-diazabicyclo[3.2.1]octan-8-yl)-6,7-dihydrothiazolo[5,4-c]pyridin-5(4H)-yl)(spiro[2.4]heptan-1-yl)methanone

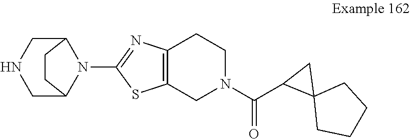 [SMILES: O=C(C1CC12CCCC2)N1CCc2nc(N3C4CCC3CNC4)sc2C1]

Prepared using spiro[2.4]heptane-1-carboxylic acid as the acid component.

¹H-NMR: δ_{H} (400 MHz, CDCl₃, mixture of rotamers) 4.72 (1H, m), 4.55 (1H, m), 4.32 (0.4H, m), 4.14 (2H, br s), 4.02-3.93 (0.6H, m), 3.82-3.72 (0.6H, m), 3.36 (0.4H, m), 3.29-3.20 (2H, m), 2.86-2.63 (4H, m), 2.12-1.93 (4H, m), 1.82-1.61 (6H, m), 1.58-1.32 (4H, m), 0.93 (1H, m); UPLC (Method A): t_{R} 1.52 min, 99%, MS (ESI) 373.2 (M+H)⁺.

### Example 163: Synthesis of 1-(2-(3,8-diazabicyclo[3.2.1]octan-8-yl)-6,7-dihydrothiazolo[5,4-c]pyridin-5(4H)-yl)-2-(2-isopropoxyphenoxy)ethan-1-one

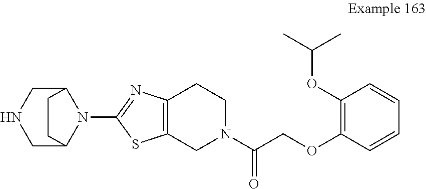 [SMILES: CC(C)Oc1ccccc1OCC(=O)N1CCc2nc(N3C4CCC3CNC4)sc2C1]

Prepared using 2-(2-isopropoxyphenoxy)acetic acid as the acid component.

¹H-NMR: δ_{H} (400 MHz, CDCl₃, mixture of rotamers) 7.02-6.84 (4H, m), 4.82-4.74 (2H, m), 4.74-4.69 (0.7H, m), 4.67-4.60 (1.3H, m), 4.56 (1H, h, J 6.0), 4.19-4.07 (2H, m), 4.00-3.85 (2H, m), 3.28-3.15 (2H, m), 2.81-2.70 (2H, m), 2.70-2.60 (2H, m), 2.12-1.98 (2H, m), 1.98-1.88 (2H, m), 1.36 (6H, d, J 6.1); UPLC (Method A): t_{R} 0.86 min, 99%, MS (ESI) 443.2 (M+H)⁺.

### Example 164: Synthesis of 1-(2-(3,8-diazabicyclo[3.2.1]octan-8-yl)-6,7-dihydrothiazolo[5,4-c]pyridin-5(4H)-yl)-2-(2-(trifluoromethoxy)phenoxy)ethan-1-one

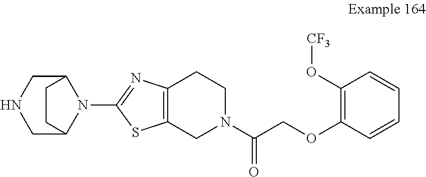 [SMILES: O=C(COc1ccccc1OC(F)(F)F)N1CCc2nc(N3C4CCC3CNC4)sc2C1]

Prepared using 2-(2-(trifluoromethoxy)phenoxy)acetic acid as the acid component.

¹H-NMR: δ_{H} (400 MHz, CDCl₃, mixture of rotamers) 7.28-7.26 (0.5H, m), 7.26-7.21 (1.5H, m), 7.14-7.05 (1H, m), 7.05-6.94 (1H, m), 4.88-4.75 (2H, m), 4.69-4.56 (2H, m), 4.19-4.05 (2H, m), 3.97-3.82 (2H, m), 3.29-3.14 (2H, m), 2.79-2.69 (2H, m), 2.69-2.62 (2H, m), 2.13-1.98 (2H, m), 1.98-1.86 (2H, m); UPLC (Method A): t_{R} 0.92 min, 99%, MS (ESI) 469.1 (M+H)⁺.

### Example 165: Synthesis of 1-(2-(3,8-diazabicyclo[3.2.1]octan-8-yl)-6,7-dihydrothiazolo[5,4-c]pyridin-5(4H)-yl)-2-(6-fluoropyridin-3-yl)ethan-1-one

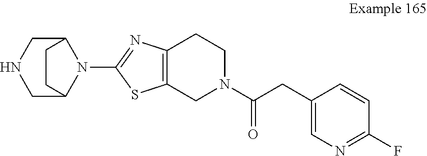 [SMILES: O=C(Cc1ccc(F)nc1)N1CCc2nc(N3C4CCC3CNC4)sc2C1]

Prepared using 2-(6-fluoropyridin-3-yl)acetic acid as the acid component.

¹H-NMR: δ_{H} (400 MHz, CDCl₃, mixture of rotamers) 8.07 (1H, dd, J 16.1, 2.4), 7.79-7.69 (1H, m), 6.95-6.86 (1H, m), 4.69-4.50 (2H, m), 4.17-4.08 (2H, m), 3.96-3.72 (4H, m), 3.28-3.14 (2H, m), 2.75-2.61 (4H, m), 2.14-1.88 (4H, m); UPLC (Method A): t_{R} 0.62 min, 100%, MS (ESI) 388.2 (M+H)⁺.

### Example 166: Synthesis of 1-(2-(3,8-diazabicyclo[3.2.1]octan-8-yl)-6,7-dihydrothiazolo[5,4-c]pyridin-5(4H)-yl)-2,2-difluoro-2-(6-fluoropyridin-3-yl)ethan-1-one

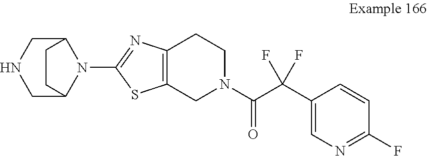 [SMILES: O=C(N1CCc2nc(N3C4CCC3CNC4)sc2C1)C(F)(F)c1ccc(F)nc1]

Prepared using 2,2-difluoro-2-(6-fluoropyridin-3-yl)acetic acid as the acid component.

¹H-NMR: δ_{H} (400 MHz, CDCl₃, mixture of rotamers) 8.51-8.37 (1H, m), 8.04-7.86 (1H, m), 7.11-6.96 (1H, m), 4.69 (2H, d, J 11.6), 4.13 (2H, s), 4.01-3.91 (2H, m), 3.22 (2H, d, J 12.3), 2.82-2.62 (4H, m), 2.12-1.90 (4H, m); UPLC (Method A): t_{R} 2.04 min, 99%, MS (ESI) 424.2 (M+H)⁺.

### Example 167: Synthesis of 2-(2-(2-(3,8-diazabicyclo[3.2.1]octan-8-yl)-6,7-dihydrothiazolo[5,4-c]pyridin-5(4H)-yl)-2-oxoethoxy)benzonitrile

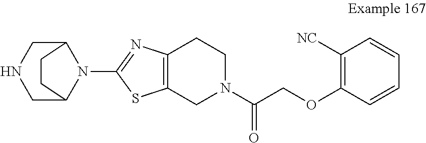 [SMILES: N#Cc1ccccc1OCC(=O)N1CCc2nc(N3C4CCC3CNC4)sc2C1]

Prepared using 2-(2-cyanophenoxy)acetic acid as the acid component.

¹H-NMR: δ_{H} (400 MHz, CDCl₃, mixture of rotamers) 7.62-7.50 (2H, m), 7.14-7.03 (2H, m), 4.97-4.87 (2H, m), 4.77-4.59 (2H, m), 4.32 (2H, s), 3.98-3.85 (2H, m), 3.50-3.38 (2H, m), 3.17-3.06 (2H, m), 2.84-2.66 (2H, m), 2.35-2.18 (4H, m); UPLC (Method A): t_{R} 1.39 min, 100%, MS (ESI) 410.2 (M+H)⁺.

### Example 168: Synthesis of 2-(2-(2-(3,8-diazabicyclo[3.2.1]octan-8-yl)-6,7-dihydrothiazolo[5,4-c]pyridin-5(4H)-yl)-2-oxoethoxy)-3-methoxybenzonitrile

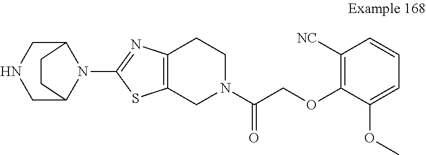 [SMILES: COc1cccc(C#N)c1OCC(=O)N1CCc2nc(N3C4CCC3CNC4)sc2C1]

Prepared using 2-(2-cyano-6-methoxyphenoxy)acetic acid as the acid component.

¹H-NMR: δ_{H} (400 MHz, CDCl₃, mixture of rotamers) 7.20-7.07 (3H, m), 4.98-4.88 (2H, m), 4.80-4.62 (2H, m), 4.18-4.09 (2H, m), 3.94 (2H, t, J 5.8), 3.90-3.81 (3H, m), 3.28-3.18 (2H, m), 2.85-2.71 (2H, m), 2.71-2.62 (2H, m), 2.13-1.90 (4H, m); UPLC (Method A): t_{R} 1.45 min, 99%, MS (ESI) 440.2 (M+H)⁺.

### Example 169: Synthesis of 1-(2-(3,8-diazabicyclo[3.2.1]octan-8-yl)-6,7-dihydrothiazolo[5,4-c]pyridin-5(4H)-yl)-2-(cyclopentyloxy)propan-1-one

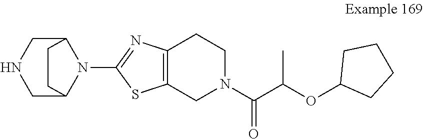 [SMILES: CC(OC1CCCC1)C(=O)N1CCc2nc(N3C4CCC3CNC4)sc2C1]

Prepared using 2-(cyclopentyloxy)propanoic acid as the acid component.

¹H-NMR: δ_{H} (400 MHz, CDCl₃, mixture of rotamers) 4.92-4.69 (0.8H, m), 4.68-4.52 (1.2H, m), 4.38-4.24 (1H, m), 4.17-4.09 (2.6H, m), 4.01-3.80 (2.4H, m), 3.24 (2H, dd, J 12.5, 1.7), 2.79-2.64 (4H, m), 2.11-1.93 (4H, m), 1.83-1.57 (6H, m), 1.55-1.46 (2H, m), 1.43 (1.8H, d, J 6.9), 1.38 (1.2H, d, J 6.9); UPLC (Method A): t_{R} 1.45 min, 99%, MS (ESI) 391.2 (M+H)⁺.

### Example 170: Synthesis of (2S)-1-(2-(3,8-diazabicyclo[3.2.1]octan-8-yl)-6,7-dihydrothiazolo[5,4-c]pyridin-5(4H)-yl)-2-cyclopentyl-2-hydroxyethan-1-one

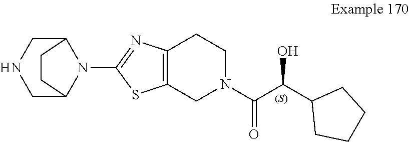

Prepared using(S)-2-cyclopentyl-2-hydroxyacetic acid as the acid component.

¹H-NMR: δ_{H} (400 MHz, CDCl₃, mixture of rotamers) 4.73-4.41 (3H, m), 4.18-4.10 (2H, m), 4.10-3.78 (1H, m), 3.78-3.59 (2H, m), 3.22 (2H, d, J 12.2), 2.84-2.60 (4H, m), 2.16-1.98 (3H, m), 1.98-1.90 (2H, m), 1.81-1.62 (4H, m), 1.54-1.34 (4H, m); UPLC (Method A): t_{R} 1.38 min, 100%, MS (ESI) 377.2 (M+H)⁺.

### Example 171: Synthesis of (2R)-1-(2-(3,8-diazabicyclo[3.2.1]octan-8-yl)-6,7-dihydrothiazolo[5,4-c]pyridin-5(4H)-yl)-2-cyclopentyl-2-hydroxyethan-1-one

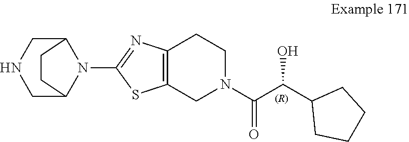

Prepared using (R)-2-cyclopentyl-2-hydroxyacetic acid as the acid component.

¹H-NMR: δ_{H} (400 MHz, CDCl₃, mixture of rotamers) 4.71-4.42 (3H, m), 4.16-4.10 (2H, m), 4.09-3.78 (1H, m), 3.78-3.52 (2H, m), 3.22 (2H, d, J 12.1), 2.82-2.63 (4H, m), 2.16-2.00 (3H, m), 1.99-1.90 (2H, m), 1.83-1.64 (4H, m), 1.54-1.33 (4H, m); UPLC (Method A): t_{R} 1.37 min, 100%, MS (ESI) 377.2 (M+H)⁺.

### Example 172: Synthesis of 1-(2-(3,8-diazabicyclo[3.2.1]octan-8-yl)-6,7-dihydrothiazolo[5,4-c]pyridin-5(4H)-yl)-2-(2,6-dimethoxyphenoxy)ethan-1-one

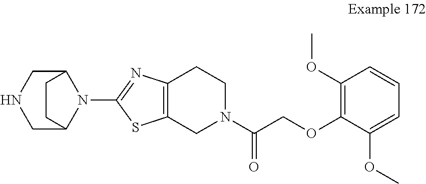 [SMILES: COc1cccc(OC)c1OCC(=O)N1CCc2nc(N3C4CCC3CNC4)sc2C1]

Prepared using 2-(2,6-dimethoxyphenoxy)acetic acid as the acid component.

¹H-NMR: δ_{H} (400 MHz, CDCl₃, mixture of rotamers) 7.02 (1H, t, J8.4), 6.57 (2H, d, J8.4), 4.98-4.60 (4H, m), 4.16 (2H, s), 4.08 (1.2H, t, J 5.7), 3.93 (0.8H, t, J 5.8), 3.84 (3.6H, s), 3.80 (2.4H, s), 3.26 (2H, d, J 12.3), 2.89-2.68 (4H, m), 2.16-2.02 (2H, m), 1.98 (2H, t, J 6.7); UPLC (Method A): t_{R} 1.48 min, 98%, MS (ESI) 445.2 (M+H)⁺.

### Example 173: Synthesis of 1-(2-(3,8-diazabicyclo[3.2.1]octan-8-yl)-6,7-dihydrothiazolo[5,4-c]pyridin-5(4H)-yl)-2-(2-(difluoromethyl)phenoxy)ethan-1-one

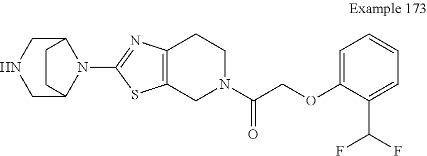 [SMILES: O=C(COc1ccccc1C(F)F)N1CCc2nc(N3C4CCC3CNC4)sc2C1]

Prepared using 2-(2-(difluoromethyl)phenoxy)acetic acid as the acid component.

¹H-NMR: δ_{H} (400 MHz, CDCl₃, mixture of rotamers) 7.58 (1H, d, J7.6), 7.42 (1H, t, J 8.0), 7.15-6.83 (3H, m), 4.89-4.78 (2H, m), 4.67-4.56 (2H, m), 4.17-4.05 (2H, m), 3.94-3.80 (2H, m), 3.28-3.15 (2H, m), 2.77-2.69 (2H, m), 2.69-2.62 (2H, m), 2.11-1.89 (4H, m); UPLC (Method A): t_{R} 1.64 min, 97%, MS (ESI) 435.2 (M+H)⁺.

### Example 174: Synthesis of 1-(2-(3,8-diazabicyclo[3.2.1]octan-8-yl)-6,7-dihydrothiazolo[5,4-c]pyridin-5(4H)-yl)-3,3-dimethylpentan-1-one

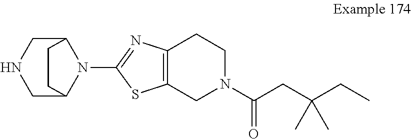 [SMILES: CCC(C)(C)CC(=O)N1CCc2nc(N3C4CCC3CNC4)sc2C1]

Prepared using 3,3-dimethylpentanoic acid as the acid component.

¹H-NMR: δ_{H} (400 MHz, CDCl₃, mixture of rotamers) 4.66-4.49 (2H, m), 4.13 (2H, s), 3.95-3.75 (2H, m), 3.22 (2H, d, J 12.4), 2.78-2.62 (4H, m), 2.37-2.25 (2H, m), 2.13-1.88 (4H, m), 1.47-1.35 (2H, m), 1.08-0.96 (6H, m), 0.91-0.82 (3H, m); UPLC (Method A): t_{R} 1.30 min, 99%, MS (ESI) 363.2 (M+H)⁺.

### Example 175: Synthesis of 1-(2-(3,8-diazabicyclo[3.2.1]octan-8-yl)-6,7-dihydrothiazolo[5,4-c]pyridin-5(4H)-yl)-4,4-difluoro-3,3-dimethylbutan-1-one

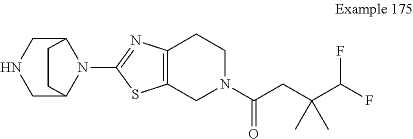 [SMILES: CC(C)(CC(=O)N1CCc2nc(N3C4CCC3CNC4)sc2C1)C(F)F]

Prepared using 4,4-difluoro-3,3-dimethylbutanoic acid as the acid component.

¹H-NMR: δ_{H} (400 MHz, CDCl₃, mixture of rotamers) 6.13 (0.26H, d, J 11.4), 5.99 (0.43H, d, J 11.6), 5.84 (0.26H, d, J 11.4), 4.63-4.58 (1.23H, m), 4.52-4.47 (0.71H, m), 4.16-4.09 (2H, m), 3.88 (0.72H, t, J5.8), 3.75 (1.25H, t, J5.8), 3.22 (2H, d, J 12.2), 2.76-2.60 (4H, m), 2.49-2.45 (2H, m), 2.09-1.89 (4H, m), 1.16-1.13 (6H, m); UPLC (Method A): t_{R} 2.05 min, 99%, MS (ESI) 385.4 (M+H)⁺.

### Example 176: Synthesis of (2R)-1-(2-(3,8-diazabicyclo[3.2.1]octan-8-yl)-6,7-dihydrothiazolo[5,4-c]pyridin-5(4H)-yl)-2-hydroxy-3,3-dimethylbutan-1-one

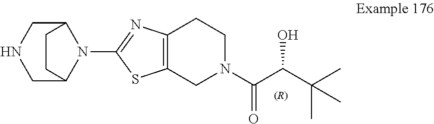

Prepared using (2R)-2-hydroxy-3,3-dimethylbutanoic acid as the acid component.

¹H-NMR: δ_{H} (400 MHz, CDCl₃, mixture of rotamers) 4.90-4.42 (2H, m), 4.35-4.17 (1H, m), 4.17-4.07 (2H, m), 4.06-3.69 (2H, m), 3.48-3.28 (1H, m), 3.22 (2H, dd, J 12.3, 1.9), 2.83-2.57 (4H, m), 2.14-1.85 (4H, m), 1.09-0.87 (9H, m); UPLC (Method A): t_{R} 1.81 min, 100%, MS (ESI) 365.2 (M+H)⁺.

### Example 177: Synthesis of (2R)-1-(2-(3,8-diazabicyclo[3.2.1]octan-8-yl)-6,7-dihydrothiazolo[5,4-c]pyridin-5(4H)-yl)-2-cyclohexyl-2-hydroxyethan-1-one

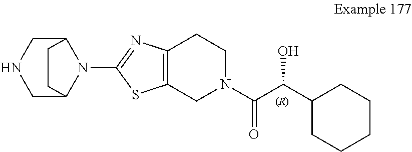

Prepared using (R)-hexahydromandelic acid as the acid component.

¹H-NMR: δ_{H} (400 MHz, CDCl₃, mixture of rotamers) 4.73-4.36 (2H, m), 4.35-4.20 (1H, m), 4.18-4.05 (2H, m), 3.85-3.51 (3H, m), 3.29-3.13 (2H, m), 2.84-2.59 (4H, m), 2.15-1.87 (4H, m), 1.86-1.63 (4H, m), 1.56-1.33 (3H, m), 1.32-1.00 (4H, m); UPLC (Method A): t_{R} 2.20 min, 100%, MS (ESI) 391.2 (M+H)⁺.

### Example 178: Synthesis of 4-(2-(3,8-diazabicyclo[3.2.1]octan-8-yl)-6,7-dihydrothiazolo[5,4-c]pyridin-5(4H)-yl)-2,2-dimethyl-4-oxobutanenitrile

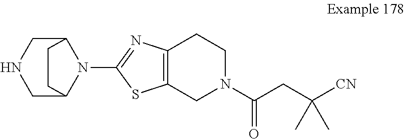 [SMILES: CC(C)(C#N)CC(=O)N1CCc2nc(N3C4CCC3CNC4)sc2C1]

Prepared using 3-cyano-3-methylbutanoic acid as the acid component.

¹H-NMR: δ_{H} (400 MHz, CDCl₃, mixture of rotamers) 4.67-4.44 (2H, m), 4.16-4.09 (2H, m), 3.96-3.70 (2H, m), 3.22 (2H, dd, J 12.4, 1.8), 2.78-2.60 (6H, m), 2.10-1.89 (4H, m), 1.52 (6H, d, J 4.7); UPLC (Method A): t_{R} 1.47 min, 99%, MS (ESI) 360.2 (M+H)⁺.

### Example 179: Synthesis of (2-(3,8-diazabicyclo[3.2.1]octan-8-yl)-6,7-dihydrothiazolo[5,4-c]pyridin-5(4H)-yl)(2,3-dihydrobenzofuran-2-yl)methanone

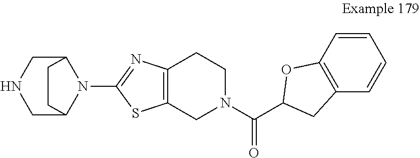 [SMILES: O=C(C1Cc2ccccc2O1)N1CCc2nc(N3C4CCC3CNC4)sc2C1]

Prepared using 2,3-dihydrobenzo[b]furan-2-carboxylic acid as the acid component.

¹H-NMR: δ_{H} (400 MHz, CDCl₃, mixture of rotamers) 7.23-7.17 (1H, m), 7.16-7.08 (1H, m), 6.92-6.84 (1H, m), 6.84-6.76 (1H, m), 5.52-5.37 (1H, m), 4.74 (1.3H, d, J 15.1), 4.59 (0.7H, d, J 16.2), 4.26-4.10 (2.3H, m), 4.10-3.97 (0.7H, m), 3.93-3.77 (1.7H, m), 3.76-3.63 (0.3H, m), 3.42-3.29 (1H, m), 3.24 (2H, d, J 11.8), 2.89 (0.7H, s), 2.85-2.63 (3.3H, m), 2.14-1.90 (4H, m); UPLC (Method A): t_{R} 2.22 min, 100%, MS (ESI) 397.2 (M+H)⁺.

### Example 180: Synthesis of (2-(3,8-diazabicyclo[3.2.1]octan-8-yl)-6,7-dihydrothiazolo[5,4-c]pyridin-5(4H)-yl)(2,3-dihydrobenzo[b][1,4]dioxin-2-yl)methanone

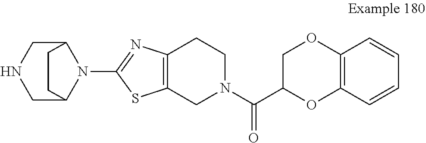 [SMILES: O=C(C1COc2ccccc2O1)N1CCc2nc(N3C4CCC3CNC4)sc2C1]

Prepared using 1,4-benzodioxan-2-carboxylic acid as the acid component.

¹H-NMR: δ_{H} (400 MHz, CDCl₃, mixture of rotamers) 6.97-6.83 (4H, m), 4.95-4.90 (0.7H, m), 4.88 (0.3H, d, J 7.9), 4.83-4.64 (1.3H, m), 4.59-4.45 (1.7H, m), 4.40-4.30 (1H, m), 4.23-4.10 (2.3H, m), 4.10-4.01 (0.7H, m), 3.89-3.78 (0.7H, m), 3.75-3.65 (0.3H, m), 3.28-3.16 (2H, m), 2.96 (0.7H, d, J 15.4), 2.84-2.71 (1.3H, m), 2.71-2.61 (2H, m), 2.14-1.98 (2H, m), 1.95 (2H, t, J 6.6); UPLC (Method A): t_{R} 2.38 min, 99%, MS (ESI) 413.2 (M+H)⁺.

### Example 181: Synthesis of (2-(3,8-diazabicyclo[3.2.1]octan-8-yl)-6,7-dihydrothiazolo[5,4-c]pyridin-5(4H)-yl)(chroman-2-yl)methanone

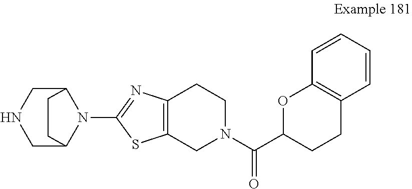 [SMILES: O=C(C1CCc2ccccc2O1)N1CCc2nc(N3C4CCC3CNC4)sc2C1]

Prepared using chromane-2-carboxylic acid as the acid component.

¹H-NMR: δ_{H} (400 MHz, CDCl₃, mixture of rotamers) 7.15-7.04 (2H, m), 6.93-6.82 (2H, m), 4.88-4.83 (0.7H, m), 4.83-4.79 (0.3H, m), 4.79-4.65 (1.3H, m), 4.60-4.52 (0.7H, m), 4.20-4.07 (2.3H, m), 4.07-3.96 (0.7H, m), 3.91-3.79 (0.7H, m), 3.79-3.68 (0.3H, m), 3.28-3.17 (2H, m), 2.98-2.84 (2.7H, m), 2.84-2.70 (1.3H, m), 2.70-2.61 (2H, m), 2.32-2.17 (2H, m), 2.12-2.02 (2H, m), 2.02-1.89 (2H, m); UPLC (Method A): t_{R} 2.52 min, 100%, MS (ESI) 411.2 (M+H)⁺.

### Example 182: Synthesis of (2-(3,8-diazabicyclo[3.2.1]octan-8-yl)-6,7-dihydrothiazolo[5,4-c]pyridin-5(4H)-yl)(chroman-3-yl)methanone

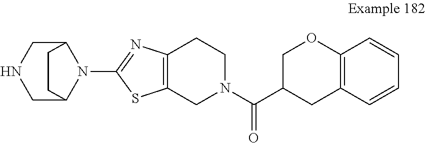 [SMILES: O=C(C1COc2ccccc2C1)N1CCc2nc(N3C4CCC3CNC4)sc2C1]

Prepared using 3-chromanecarboxylic acid as the acid component.

¹H-NMR: δ_{H} (400 MHz, CDCl₃, mixture of rotamers) 7.16-7.05 (2H, m), 6.92-6.81 (2H, m), 4.70-4.58 (2H, m), 4.45-4.34 (1H, m), 4.19-4.01 (3H, m), 4.01-3.89 (0.7H, m), 3.89-3.82 (1.3H, m), 3.34-3.12 (4H, m), 2.92-2.70 (3H, m), 2.70-2.62 (2H, m), 2.15-2.02 (2H, m), 1.99-1.91 (2H, m); UPLC (Method A): t_{R} 2.53 min, 99%, MS (ESI) 411.2 (M+H)⁺.

### Example 183: Synthesis of (2-(3,8-diazabicyclo[3.2.1]octan-8-yl)-6,7-dihydrothiazolo[5,4-c]pyridin-5(4H)-yl)(isochroman-3-yl)methanone

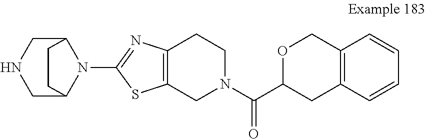 [SMILES: O=C(C1Cc2ccccc2CO1)N1CCc2nc(N3C4CCC3CNC4)sc2C1]

Prepared using 3,4-dihydro-1H-2-benzopyran-3-carboxylic acid as the acid component.

¹H-NMR: δ_{H} (400 MHz, CDCl₃, mixture of rotamers) 7.23-7.15 (3H, m), 7.04-6.98 (1H, m), 4.98-4.79 (2H, m), 4.79-4.58 (2H, m), 4.58-4.48 (1H, m), 4.18-4.04 (2.3H, m), 3.96-3.75 (1.7H, m), 3.42-3.27 (1H, m), 3.23 (2H, d, J 12.2), 2.95-2.80 (1.7H, m), 2.80-2.71 (1.3H, m), 2.67 (2H, d, J 12.3), 2.14-2.01 (2H, m), 1.98-1.89 (2H, m); UPLC (Method A): t_{R} 2.36 min, 100%, MS (ESI) 411.2 (M+H)⁺.

### Example 184: Synthesis of (2-(3,8-diazabicyclo[3.2.1]octan-8-yl)-6,7-dihydrothiazolo[5,4-c]pyridin-5(4H)-yl)(2,3-dihydrobenzofuran-3-yl)methanone

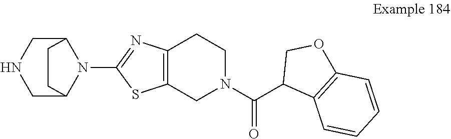 [SMILES: O=C(C1COc2ccccc21)N1CCc2nc(N3C4CCC3CNC4)sc2C1]

Prepared using 2,3-dihydrobenzofuran-3-carboxylic acid as the acid component.

¹H-NMR: δ_{H} (400 MHz, CDCl₃, mixture of rotamers) 7.21-7.12 (1.5H, m), 6.99 (0.5H, m), 6.90-6.78 (2H, m), 5.02-4.80 (2H, m), 4.76-4.63 (2.5H, m), 4.50 (0.5H, d, J 16.3), 4.26-4.04 (3H, m), 3.95-3.70 (1H, m), 3.30-3.20 (2H, m), 2.86-2.64 (4H, m), 2.14-2.04 (2H, m), 2.02-1.94 (2H, m); UPLC (Method A): t_{R} 1.39 min, 99%, MS (ESI) 397.2 (M+H)⁺.

### Example 185: Synthesis of (2-(3,8-diazabicyclo[3.2.1]octan-8-yl)-6,7-dihydrothiazolo[5,4-c]pyridin-5(4H)-yl)(benzofuran-2-yl)methanone

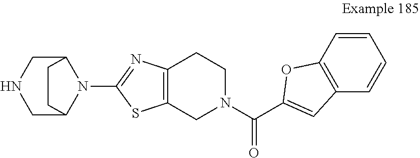 [SMILES: O=C(c1cc2ccccc2o1)N1CCc2nc(N3C4CCC3CNC4)sc2C1]

Prepared using benzofuran-2-carboxylic acid as the acid component.

¹H-NMR: δ_{H} (400 MHz, CDCl₃, mixture of rotamers) 7.67 (1H, d, J 7.8), 7.55 (1H, d, J 8.4), 7.46-7.38 (1H, m), 7.35 (1H, s), 7.33-7.27 (1H, m), 5.00-4.72 (2H, m), 4.24-3.99 (4H, m), 3.24 (2H, d, J 12.2), 2.88 (2H, s), 2.73 (2H, d, J 12.4), 2.16-1.91 (4H, m); UPLC (Method A): t_{R} 2.57 min, 100%, MS (ESI) 395.2 (M+H)⁺.

### Example 186: Synthesis of (2-(3,8-diazabicyclo[3.2.1]octan-8-yl)-6,7-dihydrothiazolo[5,4-c]pyridin-5(4H)-yl)(isoquinolin-1-yl)methanone

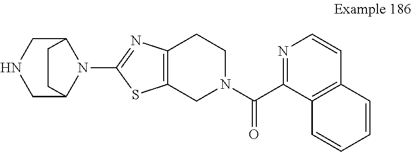 [SMILES: O=C(c1nccc2ccccc12)N1CCc2nc(N3C4CCC3CNC4)sc2C1]

Prepared using isoquinoline-1-carboxylic acid as the acid component.

¹H-NMR: δ_{H} (400 MHz, CDCl₃, mixture of rotamers) 8.59-8.49 (1H, m), 8.05 (1H, d, J 8.4), 7.93-7.84 (1H, m), 7.78-7.68 (2H, m), 7.66-7.56 (1H, m), 5.03-4.89 (1.3H, m), 4.35-4.27 (0.7H, m), 4.27-4.19 (0.7H, m), 4.19-4.11 (1.3H, m), 4.11-4.03 (0.7H, m), 3.60-3.47 (1.3H, m), 3.28-3.21 (1.3H, m), 3.21-3.15 (0.7H, m), 2.98-2.87 (0.7H, m), 2.72-2.57 (3.3H, m), 2.14-1.99 (2H, m), 1.99-1.87 (2H, m); UPLC (Method A): t_{R} 1.93 min, 99%, MS (ESI) 406.2 (M+H)⁺.

### Example 187: Synthesis of (2-(3,8-diazabicyclo[3.2.1]octan-8-yl)-6,7-dihydrothiazolo[5,4-c]pyridin-5(4H)-yl)(benzofuran-3-yl)methanone

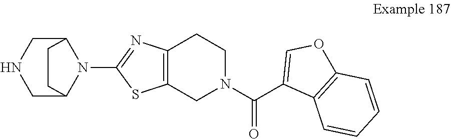 [SMILES: O=C(c1coc2ccccc12)N1CCc2nc(N3C4CCC3CNC4)sc2C1]

Prepared using 1-benzofuran-3-carboxylic acid as the acid component.

¹H-NMR: δ_{H} (400 MHz, CDCl₃) 7.90 (1H, s), 7.69 (1H, d, J7.7), 7.55 (1H, d, J8.1), 7.41-7.28 (2H, m), 4.74 (2H, br s), 4.14 (2H, s), 3.95 (2H, br s), 3.23 (2H, d, J 12.2), 2.80 (2H, s), 2.68 (2H, dd, J 12.3, 2.3), 2.13-2.03 (2H, m), 1.99-1.92 (2H, m).; UPLC (Method A): t_{R} 1.51 min, 99%, MS (ESI) 395.2 (M+H)⁺.

### Example 188: Synthesis of (2-(3,8-diazabicyclo[3.2.1]octan-8-yl)-6,7-dihydrothiazolo[5,4-c]pyridin-5(4H)-yl)(1H-indol-2-yl)methanone

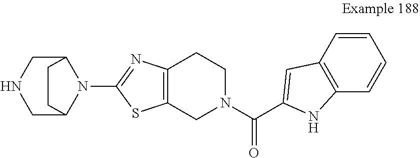 [SMILES: O=C(c1cc2ccccc2[nH]1)N1CCc2nc(N3C4CCC3CNC4)sc2C1]

Prepared using indole-2-carboxylic acid as the acid component.

¹H-NMR: δ_{H} (400 MHz, CDCl₃) 9.15 (1H, s), 7.73-7.62 (1H, m), 7.48-7.38 (1H, m), 7.33-7.27 (1H, m), 7.20-7.10 (1H, m), 6.92-6.82 (1H, m), 4.91 (2H, s), 4.34-4.02 (4H, m), 3.31-3.15 (2H, m), 2.88 (2H, s), 2.76-2.59 (2H, m), 2.14-1.99 (2H, m), 1.99-1.89 (2H, m); UPLC (Method A): t_{R} 1.58 min, 98%, MS (ESI) 394.2 (M+H)⁺.

### Example 189: Synthesis of (2-(3,8-diazabicyclo[3.2.1]octan-8-yl)-6,7-dihydrothiazolo[5,4-c]pyridin-5(4H)-yl)(1-methyl-1H-indol-2-yl)methanone

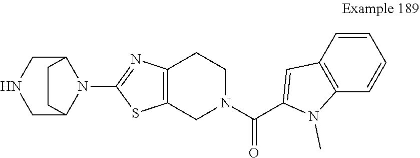 [SMILES: Cn1c(C(=O)N2CCc3nc(N4C5CCC4CNC5)sc3C2)cc2ccccc21]

Prepared using 1-methylindole-2-carboxylic acid as the acid component.

¹H-NMR: δ_{H} (400 MHz, CDCl₃) 7.69-7.60 (1H, m), 7.41-7.35 (1H, m), 7.35-7.28 (1H, m), 7.19-7.12 (1H, m), 6.72-6.64 (1H, m), 4.84-4.73 (2H, m), 4.20-4.09 (2H, m), 4.09-3.95 (2H, m), 3.85 (3H, s), 3.24 (2H, d, J 12.2), 2.87-2.75 (2H, m), 2.75-2.64 (2H, m), 2.14-2.00 (2H, m), 2.00-1.91 (2H, m); UPLC (Method A): t_{R} 1.65 min, 97%, MS (ESI) 408.2 (M+H)⁺.

### Example 190: Synthesis of (2-(3,8-diazabicyclo[3.2.1]octan-8-yl)-6,7-dihydrothiazolo[5,4-c]pyridin-5(4H)-yl)(benzo[d]oxazol-2-yl)methanone

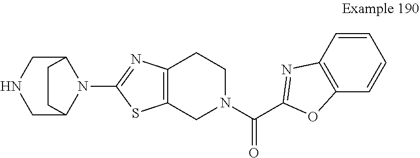 [SMILES: O=C(c1nc2ccccc2o1)N1CCc2nc(N3C4CCC3CNC4)sc2C1]

Prepared using benzo[d]oxazole-2-carboxylic acid as the acid component.

¹H-NMR: δ_{H} (400 MHz, CDCl₃, mixture of rotamers) 7.89-7.81 (1H, m), 7.69-7.63 (1H, m), 7.53-7.40 (2H, m), 5.27-5.18 (0.8H, m), 4.91-4.79 (1.2H, m), 4.47-4.36 (1.2H, m), 4.29-4.04 (2.8H, m), 3.43-3.08 (1.8H, m), 2.95-2.89 (1.2H, m), 2.89-2.83 (0.8H, m), 2.79-2.62 (1.8H, m), 2.14-2.00 (2H, m), 2.00-1.87 (2H, m); UPLC (Method A): t_{R} 1.49 min, 100%, MS (ESI) 396.2 (M+H)⁺.

### Example 192: Synthesis of (2-(3,8-diazabicyclo[3.2.1]octan-8-yl)-6,7-dihydrothiazolo[5,4-c]pyridin-5(4H)-yl)(isoquinolin-3-yl)methanone

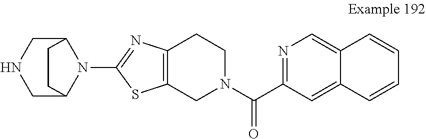 [SMILES: O=C(c1cc2ccccc2cn1)N1CCc2nc(N3C4CCC3CNC4)sc2C1]

Prepared using isoquinoline-3-carboxylic acid as the acid component.

¹H-NMR: δ_{H} (400 MHz, CDCl₃, mixture of rotamers) 9.26 (1H, s), 8.21-8.09 (1H, m), 8.04 (1H, d, J 8.0), 7.92 (1H, d, J 8.2), 7.81-7.73 (1H, m), 7.73-7.66 (1H, m), 4.90-4.68 (2H, m), 4.27-3.83 (4H, m), 3.37-3.23 (2H, m), 2.91-2.62 (4H, m), 2.18-1.99 (4H, m); UPLC (Method A): t_{R} 1.41 min, 98%, MS (ESI) 406.2 (M+H)⁺.

### Example 193: Synthesis of (2-(3,8-diazabicyclo[3.2.1]octan-8-yl)-6,7-dihydrothiazolo[5,4-c]pyridin-5(4H)-yl)(phenyl)methanone

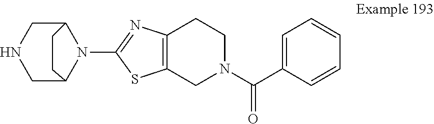 [SMILES: O=C(c1ccccc1)N1CCc2nc(N3C4CCC3CNC4)sc2C1]

Prepared using benzoic acid as the acid component.

¹H-NMR: δ_{H} (400 MHz, CDCl₃, mixture of rotamers) 7.50-7.37 (5H, m), 4.87-4.63 (1.3H, m), 4.59-4.36 (0.7H, m), 4.23-3.95 (2.7H, m), 3.79-3.59 (1.3H, m), 3.23 (2H, d, J 12.3), 2.93-2.59 (4H, m), 2.16-1.98 (2H, m), 1.94 (2H, d, J 7.6); UPLC (Method A): t_{R} 1.82 min, 99%, MS (ESI) 355.2 (M+H)⁺.

### Example 194: Synthesis of (2-(3,8-diazabicyclo[3.2.1]octan-8-yl)-6,7-dihydrothiazolo[5,4-c]pyridin-5(4H)-yl)(o-tolyl)methanone

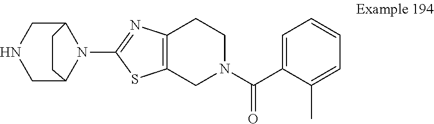 [SMILES: Cc1ccccc1C(=O)N1CCc2nc(N3C4CCC3CNC4)sc2C1]

Prepared using 2-methylbenzoic acid as the acid component.

¹H-NMR: δ_{H} (400 MHz, CDCl₃, mixture of rotamers) 7.34-7.27 (1H, m), 7.26-7.16 (3H, m), 5.03-4.61 (1.25H, m), 4.35-3.81 (3.5H, m), 3.52 (1.25H, m), 3.28-3.16 (2H, m), 2.88-2.55 (4H, m), 2.36-2.23 (3H, m), 2.11-1.89 (4H, m); UPLC (Method A): t_{R} 1.39 min, 98%, MS (ESI) 369.2 (M+H)⁺.

### Example 195: Synthesis of (2-(3,8-diazabicyclo[3.2.1]octan-8-yl)-6,7-dihydrothiazolo[5,4-c]pyridin-5(4H)-yl)(m-tolyl)methanone

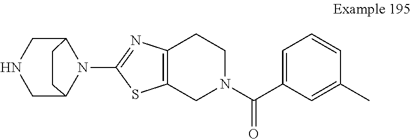 [SMILES: Cc1cccc(C(=O)N2CCc3nc(N4C5CCC4CNC5)sc3C2)c1]

Prepared using 3-methylbenzoic acid as the acid component.

¹H-NMR: δ_{H} (400 MHz, CDCl₃, mixture of rotamers) 7.34-7.20 (4H, m), 4.83-4.37 (2H, m), 4.13 (2H, s), 4.08-3.60 (2H, m), 3.31-3.09 (2H, m), 2.84-2.61 (4H, m), 2.38 (3H, s), 2.13-2.03 (2H, m), 1.98-1.87 (2H, m); UPLC (Method A): t_{R} 1.46 min, 97%, MS (ESI) 369.2 (M+H)⁺.

### Example 196: Synthesis of (2-(3,8-diazabicyclo[3.2.1]octan-8-yl)-6,7-dihydrothiazolo[5,4-c]pyridin-5(4H)-yl)(p-tolyl)methanone

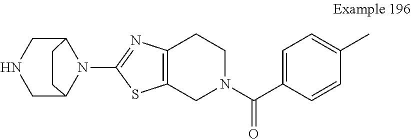 [SMILES: Cc1ccc(C(=O)N2CCc3nc(N4C5CCC4CNC5)sc3C2)cc1]

Prepared using 4-methylbenzoic acid as the acid component.

¹H-NMR: δ_{H} (400 MHz, CDCl₃, mixture of rotamers) 7.39-7.32 (2H, m), 7.23 (2H, d, J 7.8), 4.85-4.38 (2H, m), 4.13 (2H, s), 4.08-3.58 (2H, m), 3.22 (2H, d, J 12.3), 2.86-2.61 (4H, m), 2.39 (3H, s), 2.13-2.01 (2H, m), 2.00-1.89 (2H, m); UPLC (Method A): t_{R} 1.47 min, 100%, MS (ESI) 369.2 (M+H)⁺.

### Example 197: Synthesis of (2-(3,8-diazabicyclo[3.2.1]octan-8-yl)-6,7-dihydrothiazolo[5,4-c]pyridin-5(4H)-yl)(2-methoxyphenyl)methanone

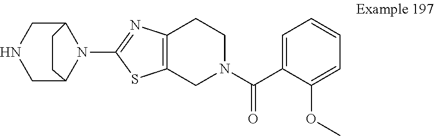 [SMILES: COc1ccccc1C(=O)N1CCc2nc(N3C4CCC3CNC4)sc2C1]

Prepared using 2-methoxybenzoic acid as the acid component.

¹H-NMR: δ_{H} (400 MHz, CDCl₃, mixture of rotamers) 7.41-7.34 (1H, m), 7.29-7.24 (1H, m), 7.03-6.89 (2H, m), 4.87-4.74 (1H, m), 4.42-4.19 (1H, m), 4.18-4.06 (2H, m), 3.82 (3H, d, J 16.4), 3.64-3.43 (1H, m), 3.28-3.16 (2H, m), 2.84-2.52 (4H, m), 2.14-1.87 (4H, m).; UPLC (Method A): t_{R} 1.32 min, 100%, MS (ESI) 385.2 (M+H)⁺.

### Example 198: Synthesis of (2-(3,8-diazabicyclo[3.2.1]octan-8-yl)-6,7-dihydrothiazolo[5,4-c]pyridin-5(4H)-yl)(4-fluorophenyl)methanone

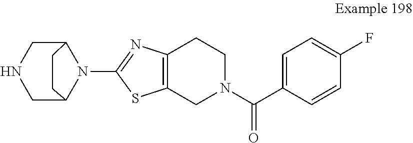 [SMILES: O=C(c1ccc(F)cc1)N1CCc2nc(N3C4CCC3CNC4)sc2C1]

Prepared using 4-fluorobenzoic acid as the acid component.

¹H-NMR: δ_{H} (400 MHz, CDCl₃, mixture of rotamers) 7.55-7.40 (2H, m), 7.22-7.04 (2H, m), 4.85-4.36 (2H, m), 4.13 (2H, s), 4.07-3.54 (2H, m), 3.31-3.04 (2H, m), 2.90-2.56 (4H, m), 2.12-1.85 (4H, m); UPLC (Method A): t_{R} 1.35 min, 100%, MS (ESI) 373.2 (M+H)⁺.

### Example 199: Synthesis of (2-(3,8-diazabicyclo[3.2.1]octan-8-yl)-6,7-dihydrothiazolo[5,4-c]pyridin-5(4H)-yl)(3-(difluoromethyl)phenyl)methanone

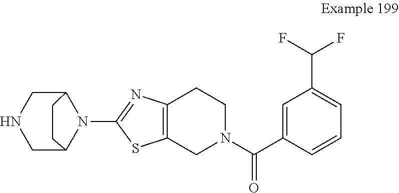 [SMILES: O=C(c1cccc(C(F)F)c1)N1CCc2nc(N3C4CCC3CNC4)sc2C1]

Prepared using 3-(difluoromethyl)benzoic acid as the acid component.

¹H-NMR: δ_{H} (400 MHz, CDCl₃, mixture of rotamers) 7.64-7.49 (4H, m), 6.67 (1H, m), 4.83-4.39 (2H, m), 4.20-4.09 (2H, m), 4.09-3.58 (2H, m), 3.29-3.14 (2H, m), 2.89-2.61 (4H, m), 2.13-2.01 (2H, m), 1.98-1.89 (2H, m); UPLC (Method A): t_{R} 1.45 min, 100%, MS (ESI) 405.2 (M+H)⁺.

### Example 200: Synthesis of (2-(3,8-diazabicyclo[3.2.1]octan-8-yl)-6,7-dihydrothiazolo[5,4-c]pyridin-5(4H)-yl)(6-fluoropyridin-3-yl)methanone

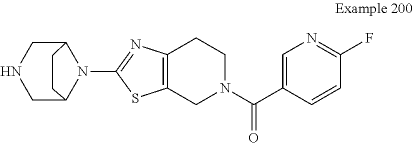 [SMILES: O=C(c1ccc(F)nc1)N1CCc2nc(N3C4CCC3CNC4)sc2C1]

Prepared using 6-fluoronicotinic acid as the acid component.

¹H-NMR: δ_{H} (400 MHz, CDCl₃, mixture of rotamers) 8.37 (1H, d, J2.5), 7.94 (1H, m), 7.03 (1H, dd, J 8.2, 2.8), 4.84-4.44 (2H, m), 4.14 (2H, br s), 4.08-3.65 (2H, m), 3.23 (2H, d, J 12.3), 2.87-2.65 (4H, m), 2.12-1.94 (4H, m); UPLC (Method A): t_{R} 1.37 min, 95%, MS (ESI) 374.2 (M+H)⁺.

### Example 201: Synthesis of (2-(3,8-diazabicyclo[3.2.1]octan-8-yl)-6,7-dihydrothiazolo[5,4-c]pyridin-5(4H)-yl)(pyridin-2-yl)methanone

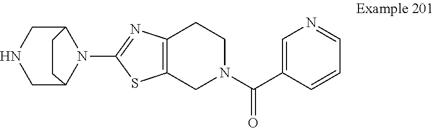 [SMILES: O=C(c1cccnc1)N1CCc2nc(N3C4CCC3CNC4)sc2C1]

Prepared using 2-picolinic acid as the acid component.

¹H-NMR: δ_{H} (400 MHz, CDCl₃, mixture of rotamers) 8.62 (1H, t, J4.5), 7.82 (1H, td, J7.7, 1.7), 7.73 (0.4H, d, J 7.8), 7.67 (1H, d, J 7.8), 7.37 (1H, dd, J 4.8, 1.2), 4.81 (1.2H, s), 4.66 (0.8H, s), 4.18-4.06 (2.8H, m), 3.83 (1.2H, t, J 5.8), 3.24 (2H, dd, J 14.1, 12.2), 2.87-2.74 (2H, m), 2.74-2.64 (2H, m), 2.12-1.93 (4H, m); UPLC (Method C): t_{R} 1.32 min, 99%, MS (ESI) 356.2 (M+H)⁺.

### Example 202: Synthesis of (2-(3,8-diazabicyclo[3.2.1]octan-8-yl)-6,7-dihydrothiazolo[5,4-c]pyridin-5(4H)-yl)(5-fluoropyridin-2-yl)methanone

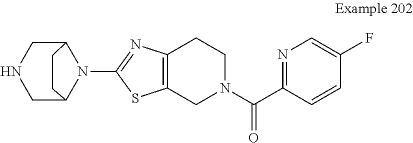 [SMILES: O=C(c1ccc(F)cn1)N1CCc2nc(N3C4CCC3CNC4)sc2C1]

Prepared using 5-fluoropicolinic acid as the acid component.

¹H-NMR: δ_{H} (400 MHz, CDCl₃, mixture of rotamers) 8.48-8.42 (1H, m), 7.82 (0.4H, dd, J8.6, 4.5), 7.77 (0.6H, dd, J 8.6, 4.5), 7.56-7.47 (1H, m), 4.79 (1.2H, t, J2.1), 4.69 (0.8H, t, J 2.1), 4.19-4.10 (2H, m), 4.07 (1.2H, t, J5.8), 3.86 (0.8H, t, J5.8), 3.23 (2H, t, J 12.8), 2.87-2.76 (2H, m), 2.73-2.63 (2H, m), 2.13-1.91 (4H, m); UPLC (Method C): t_{R} 1.42 min, 99%, MS (ESI) 374.2 (M+H)⁺.

### Example 203: Synthesis of (2-(3,8-diazabicyclo[3.2.1]octan-8-yl)-6,7-dihydrothiazolo[5,4-c]pyridin-5(4H)-yl)(2-(difluoromethoxy)phenyl)methanone

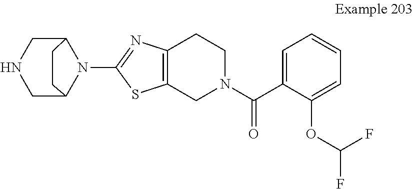 [SMILES: O=C(c1ccccc1OC(F)F)N1CCc2nc(N3C4CCC3CNC4)sc2C1]

Prepared using 2-(difluoromethoxy)benzoic acid as the acid component.

¹H-NMR: δ_{H} (400 MHz, CDCl₃, mixture of rotamers) δ_{H} (400 MHz, CDCl₃) 7.48-7.40 (1H, m), 7.40-7.34 (1H, m), 7.33-7.28 (1H, m), 7.26-7.20 (1H, m), 6.76-6.25 (1H, m), 5.04-4.57 (1H, m), 4.42-4.19 (1H, m), 4.17-4.02 (2H, m), 3.96-3.48 (2H, m), 3.22 (2H, td, J 12.0, 1.9), 2.91-2.55 (4H, m), 2.16-1.85 (4H, m); UPLC (Method A): t_{R} 0.75 min, 100%, MS (ESI) 421.2 (M+H)⁺.

### Example 204: Synthesis of (2-(3,8-diazabicyclo[3.2.1]octan-8-yl)-6,7-dihydrothiazolo[5,4-c]pyridin-5(4H)-yl)(4-(difluoromethoxy)phenyl)methanone

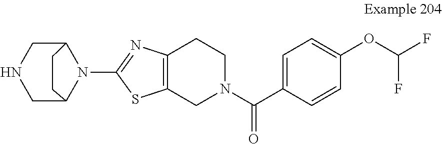 [SMILES: O=C(c1ccc(OC(F)F)cc1)N1CCc2nc(N3C4CCC3CNC4)sc2C1]

Prepared using 4-(difluoromethoxy)benzoic acid as the acid component.

¹H-NMR: δ_{H} (400 MHz, CDCl₃, mixture of rotamers) 7.52-7.45 (2H, m), 7.18 (2H, d, J 8.3), 6.76-6.35 (1H, m), 4.83-4.38 (2H, m), 4.20-3.59 (4H, m), 3.22 (2H, d, J 12.3), 2.87-2.58 (4H, m), 2.15-1.86 (4H, m); UPLC (Method A): t_{R} 0.80 min, 97%, MS (ESI) 421.2 (M+H)⁺.

### Example 205: Synthesis of (2-(3,8-diazabicyclo[3.2.1]octan-8-yl)-6,7-dihydrothiazolo[5,4-c]pyridin-5(4H)-yl)(3-(trifluoromethoxy)phenyl)methanone

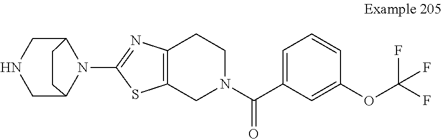 [SMILES: O=C(c1cccc(OC(F)(F)F)c1)N1CCc2nc(N3C4CCC3CNC4)sc2C1]

Prepared using 3-(trifluoromethoxy)benzoic acid as the acid component.

¹H-NMR: δ_{H} (400 MHz, CDCl₃, mixture of rotamers) 7.53-7.43 (1H, m), 7.42-7.36 (1H, m), 7.35-7.28 (2H, m), 4.81-4.39 (2H, m), 4.16 (2H, s), 4.08-3.58 (2H, m), 3.28-3.23 (2H, m), 2.88-2.65 (4H, m), 2.14-2.05 (2H, m), 2.01-1.91 (2H, m); UPLC (Method A): t_{R} 0.88 min, 95%, MS (ESI) 439.1 (M+H)⁺.

### Example 206: Synthesis of (2-(3,8-diazabicyclo[3.2.1]octan-8-yl)-6,7-dihydrothiazolo[5,4-c]pyridin-5(4H)-yl)(3-(difluoromethoxy)phenyl)methanone

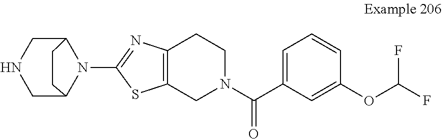 [SMILES: O=C(c1cccc(OC(F)F)c1)N1CCc2nc(N3C4CCC3CNC4)sc2C1]

Prepared using 3-(difluoromethoxy)benzoic acid as the acid component.

¹H-NMR: δ_{H} (400 MHz, CDCl₃, mixture of rotamers) 7.44 (1H, t, J 7.9), 7.33-7.27 (1H, m), 7.25-7.18 (2H, m), 6.75-6.33 (1H, m), 4.85-4.36 (2H, m), 4.13 (2H, s), 4.08-3.58 (2H, m), 3.23 (2H, d, J 12.2), 2.87-2.62 (4H, m), 2.14-1.88 (4H, m); UPLC (Method A): t_{R} 0.79 min, 99%, MS (ESI) 421.2 (M+H)⁺.

### Example 207: Synthesis of (2-(3,8-diazabicyclo[3.2.1]octan-8-yl)-6,7-dihydrothiazolo[5,4-c]pyridin-5(4H)-yl)(2-(trifluoromethoxy)phenyl)methanone

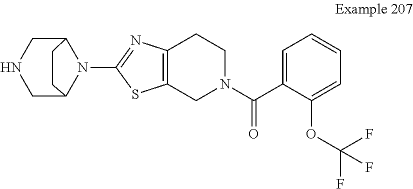 [SMILES: O=C(c1ccccc1OC(F)(F)F)N1CCc2nc(N3C4CCC3CNC4)sc2C1]

Prepared using 2-(trifluoromethoxy)benzoic acid as the acid component.

¹H-NMR: δ_{H} (400 MHz, CDCl₃, mixture of rotamers) 7.53-7.27 (4H, m), 5.07-4.54 (1H, m), 4.40-4.06 (3H, m), 3.97-3.46 (2H, m), 3.29-3.07 (2H, m), 2.89-2.55 (4H, m), 2.16-1.87 (4H, m); UPLC (Method A): t_{R} 0.82 min, 95%, MS (ESI) 439.1 (M+H)⁺.

### Example 208: Synthesis of (2-(3,8-diazabicyclo[3.2.1]octan-8-yl)-6,7-dihydrothiazolo[5,4-c]pyridin-5(4H)-yl)(4-(trifluoromethoxy)phenyl)methanone

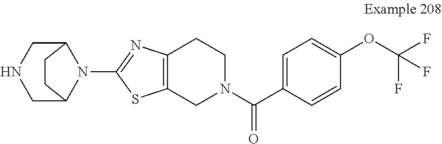 [SMILES: O=C(c1ccc(OC(F)(F)F)cc1)N1CCc2nc(N3C4CCC3CNC4)sc2C1]

Prepared using 4-(trifluoromethoxy)benzoic acid as the acid component.

¹H-NMR: δ_{H} (400 MHz, CDCl₃, mixture of rotamers) 7.55-7.47 (2H, m), 7.28 (2H, d, J 7.9), 4.86-4.37 (2H, m), 4.21-4.10 (2H, m), 4.09-3.57 (2H, m), 3.30-3.14 (2H, m), 2.94-2.60 (4H, m), 2.17-1.87 (4H, m); UPLC (Method A): t_{R} 0.89 min, 99%, MS (ESI) 439.2 (M+H)⁺.

### Example 209: Synthesis of (2-(3,8-diazabicyclo[3.2.1]octan-8-yl)-6,7-dihydrothiazolo[5,4-c]pyridin-5(4H)-yl)(2-fluorophenyl)methanone

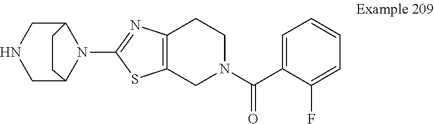 [SMILES: O=C(c1ccccc1F)N1CCc2nc(N3C4CCC3CNC4)sc2C1]

Prepared using 2-fluorobenzoic acid as the acid component.

¹H-NMR: δ_{H} (400 MHz, CDCl₃, mixture of rotamers) 7.46-7.38 (2H, m), 7.25-7.19 (1H, m), 7.17-7.09 (1H, m), 4.81 (1H, s), 4.43-4.13 (2H, m), 4.12-3.58 (2H, m), 3.29-3.15 (2H, m), 2.88-2.58 (4H, m), 2.12-1.90 (4H, m); UPLC (Method A): t_{R} 1.31 min, 96%, MS (ESI) 373.2 (M+H)⁺.

### Example 210: Synthesis of (2-(3,8-diazabicyclo[3.2.1]octan-8-yl)-6,7-dihydrothiazolo[5,4-c]pyridin-5(4H)-yl)(2-chlorophenyl)methanone

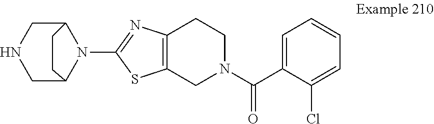 [SMILES: O=C(c1ccccc1Cl)N1CCc2nc(N3C4CCC3CNC4)sc2C1]

Prepared using 2-chlorobenzoic acid as the acid component.

¹H-NMR: δ_{H} (400 MHz, CDCl₃, mixture of rotamers) 7.46-7.29 (4H, m), 5.00-4.65 (1.3H, m), 4.38-4.17 (0.7H, m), 4.16-4.06 (2H, m), 4.01-3.86 (0.3H, m), 3.62-3.47 (1.3H, m), 3.29-3.16 (2H, m), 2.88-2.53 (4H, m), 2.12-1.88 (4H, m); UPLC (Method A): t_{R} 1.39 min, 100%, MS (ESI) 389.0 (M+H)⁺.

### Example 211: Synthesis of (2-(3,8-diazabicyclo[3.2.1]octan-8-yl)-6,7-dihydrothiazolo[5,4-c]pyridin-5(4H)-yl)(2-isopropoxyphenyl)methanone

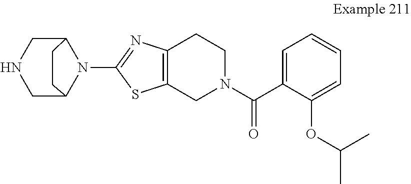 [SMILES: CC(C)Oc1ccccc1C(=O)N1CCc2nc(N3C4CCC3CNC4)sc2C1]

Prepared using 2-isopropoxybenzoic acid as the acid component.

¹H-NMR: δ_{H} (400 MHz, CDCl₃, mixture of rotamers) 7.36-7.29 (1H, m), 7.29-7.27 (0.5H, m), 7.26-7.24 (0.5H, m), 7.01-6.94 (1H, m), 6.94-6.86 (1H, m), 5.00-4.63 (1H, m), 4.62-4.44 (1H, m), 4.42-4.16 (1H, m), 4.16-3.99 (2.8H, m), 3.59-3.51 (1.2H, m), 3.29-3.16 (2H, m), 2.84-2.51 (4H, m), 2.13-1.87 (4H, m), 1.37-1.16 (6H, m); UPLC (Method A): t_{R} 1.52 min, 98%, MS (ESI) 413.2 (M+H)⁺.

### Example 212: Synthesis of (2-(3,8-diazabicyclo[3.2.1]octan-8-yl)-6,7-dihydrothiazolo[5,4-c]pyridin-5(4H)-yl)(2,4-difluorophenyl)methanone

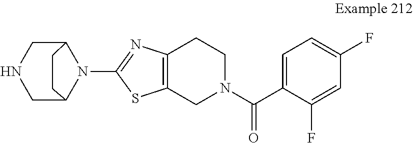 [SMILES: O=C(c1ccc(F)cc1F)N1CCc2nc(N3C4CCC3CNC4)sc2C1]

Prepared using 2,4-difluorobenzoic acid as the acid component.

¹H-NMR: δ_{H} (400 MHz, CDCl₃, mixture of rotamers) 7.60-7.45 (1H, m), 7.45-7.33 (1H, m), 7.26-7.14 (1H, m), 4.75-4.30 (2H, m), 4.04-3.47 (4H, m), 2.92 (2H, t, J 13.7), 2.66-2.51 (4H, m), 1.98-1.78 (4H, m); UPLC (Method A): t_{R} 1.37 min, 100%, MS (ESI) 391.2 (M+H)⁺.

### Example 213: Synthesis of 2-(2-(3,8-diazabicyclo[3.2.1]octan-8-yl)-4,5,6,7-tetrahydrothiazolo[5,4-c]pyridine-5-carbonyl)benzonitrile

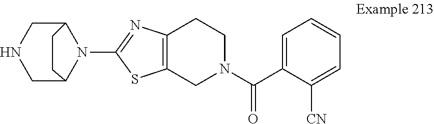 [SMILES: N#Cc1ccccc1C(=O)N1CCc2nc(N3C4CCC3CNC4)sc2C1]

Prepared using 2-cyanobenzoic acid as the acid component.

¹H-NMR: δ_{H} (400 MHz, CDCl₃, mixture of rotamers) 7.80-7.72 (1H, m), 7.72-7.63 (1H, m), 7.58-7.46 (2H, m), 4.92-4.32 (2H, m), 4.19-3.55 (4H, m), 3.22 (2H, t, J 12.0), 2.90-2.60 (4H, m), 2.16-1.88 (4H, m); UPLC (Method C): t_{R} 1.50 min, 91%, MS (ESI) 380.2 (M+H)⁺.

### Example 214: Synthesis of (2-(3,8-diazabicyclo[3.2.1]octan-8-yl)-6,7-dihydrothiazolo[5,4-c]pyridin-5(4H)-yl)(2-(methoxymethyl)phenyl)methanone

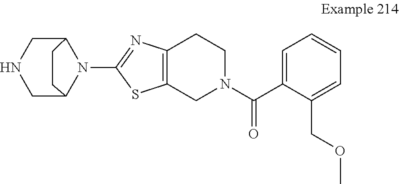 [SMILES: COCc1ccccc1C(=O)N1CCc2nc(N3C4CCC3CNC4)sc2C1]

Prepared using 2-(methoxymethyl)benzoic acid as the acid component.

¹H-NMR: δ_{H} (400 MHz, CDCl₃, mixture of rotamers) 7.50-7.29 (3H, m), 7.24 (1H, t, J 6.7), 5.01-4.30 (3.4H, m), 4.25 (1H, s), 4.20-4.07 (2H, m), 3.87-3.44 (1.6H, m), 3.36 (1.7H, s), 3.28-3.16 (3.3H, m), 2.91-2.56 (4H, m), 2.12-2.00 (2H, m), 2.00-1.90 (2H, m); UPLC (Method A): t_{R} 1.29 min, 97%, MS (ESI) 399.2 (M+H)⁺.

### Example 215: Synthesis of (2-(3,8-diazabicyclo[3.2.1]octan-8-yl)-6,7-dihydrothiazolo[5,4-c]pyridin-5(4H)-yl)(2-isopropylphenyl)methanone

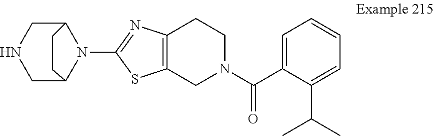 [SMILES: CC(C)c1ccccc1C(=O)N1CCc2nc(N3C4CCC3CNC4)sc2C1]

Prepared using 2-isopropylbenzoic acid as the acid component.

¹H-NMR: δ_{H} (400 MHz, CDCl₃, mixture of rotamers) 7.41-7.33 (2H, m), 7.25-7.19 (1H, m), 7.18-7.11 (1H, m), 5.02-4.62 (1.2H, m), 4.36 (0.4H, m), 4.31-4.07 (2.7H, m), 3.81 (0.4H, m), 3.61-3.43 (1.3H, m), 3.23 (2H, t, J 13.5), 3.03-2.89 (1H, m), 2.87-2.51 (4H, m), 2.14-2.01 (2H, m), 2.00-1.90 (2H, m), 1.29-1.21 (5H, m), 1.10 (1H, d, J 6.8); UPLC (Method A): t_{R} 1.63 min, 100%, MS (ESI) 397.2 (M+H)⁺.

### Example 216: Synthesis of (2-(3,8-diazabicyclo[3.2.1]octan-8-yl)-6,7-dihydrothiazolo[5,4-c]pyridin-5(4H)-yl)(4-fluoro-2-methylphenyl)methanone

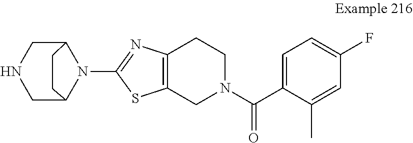 [SMILES: Cc1cc(F)ccc1C(=O)N1CCc2nc(N3C4CCC3CNC4)sc2C1]

Prepared using 4-fluoro-2-methylbenzoic acid as the acid component.

¹H-NMR: δ_{H} (400 MHz, CDCl₃, mixture of rotamers) 7.22-7.14 (1H, m), 7.00-6.87 (2H, m), 5.01-4.59 (1.2H, m), 4.36-4.04 (3.2H, m), 3.89 (0.4H, m), 3.52 (1.2H, br s), 3.23 (2H, t, J 11.1), 2.87-2.54 (4H, m), 2.33 (2H, s), 2.26 (1H, s), 2.13-2.02 (2H, m), 2.01-1.91 (2H, m); UPLC (Method A): t_{R} 1.43 min, 98%, MS (ESI) 387.2 (M+H)⁺.

### Example 217: Synthesis of (2-(3,8-diazabicyclo[3.2.1]octan-8-yl)-6,7-dihydrothiazolo[5,4-c]pyridin-5(4H)-yl)(chroman-8-yl)methanone

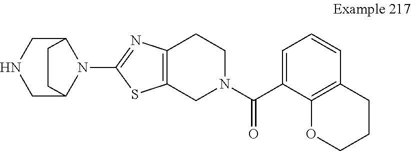 [SMILES: O=C(c1cccc2c1OCCC2)N1CCc2nc(N3C4CCC3CNC4)sc2C1]

Prepared using chromane-8-carboxylic acid as the acid component.

¹H-NMR: δ_{H} (400 MHz, CDCl₃, mixture of rotamers) 7.07 (2H, d, J 7.5), 6.91-6.82 (1H, m), 4.91-4.70 (1H, m), 4.41-4.08 (5H, m), 3.83-3.47 (2H, m), 3.22 (2H, t, J 11.4), 2.86-2.54 (6H, m), 2.13-1.88 (6H, m); UPLC (Method A): t_{R} 1.46 min, 100%, MS (ESI) 411.2 (M+H)⁺.

### Example 218: Synthesis of (2-(3,8-diazabicyclo[3.2.1]octan-8-yl)-6,7-dihydrothiazolo[5,4-c]pyridin-5(4H)-yl)(2-(difluoromethyl)phenyl)methanone

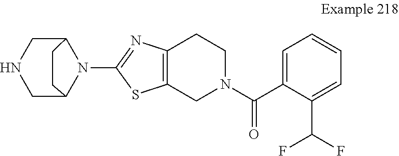 [SMILES: O=C(c1ccccc1C(F)F)N1CCc2nc(N3C4CCC3CNC4)sc2C1]

Prepared using 2-(difluoromethyl)benzoic acid as the acid component.

¹H-NMR: δ_{H} (400 MHz, CDCl₃, mixture of rotamers) 7.79-7.66 (1H, m), 7.59-7.46 (2H, m), 7.40-7.28 (1H, m), 7.07-6.67 (1H, m), 5.03-4.13 (3H, m), 4.09-3.41 (2H, m), 3.31-3.15 (2H, m), 2.89-2.57 (4H, m), 2.13-1.88 (4H, m); UPLC (Method A): t_{R} 1.40 min, 99%, MS (ESI) 405.2 (M+H)⁺.

### Example 219: Synthesis of (2-(3,8-diazabicyclo[3.2.1]octan-8-yl)-6,7-dihydrothiazolo[5,4-c]pyridin-5(4H)-yl)(2-methoxy-4-methylphenyl)methanone

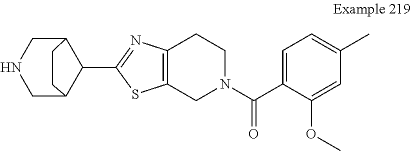 [SMILES: COc1cc(C)ccc1C(=O)N1CCc2nc(C3C4CCC3CNC4)sc2C1]

Prepared using 2-methoxy-4-methylbenzoic acid as the acid component.

¹H-NMR: δ_{H} (400 MHz, CDCl₃, mixture of rotamers) 7.18-7.12 (1H, m), 6.84-6.77 (1H, m), 6.73 (1H, d, J 7.9), 4.79 (1.3H, q, J 2.0), 4.41-4.20 (1H, m), 4.17-4.07 (2H, m), 3.82 (2H, s), 3.78 (1H, s), 3.64-3.45 (1.4H, m), 3.27-3.17 (2H, m), 2.86-2.74 (0.6H, m), 2.74-2.52 (3.5H, m), 2.38 (2H, s), 2.37 (1H, s), 2.12-1.99 (2H, m), 1.98-1.88 (2H, m); UPLC (Method A): t_{R} 2.15 min, 99%, MS (ESI) 399.2 (M+H)⁺.

### Example 220: Synthesis of (2-(3,8-diazabicyclo[3.2.1]octan-8-yl)-6,7-dihydrothiazolo[5,4-c]pyridin-5(4H)-yl)(2-cyclopropylphenyl)methanone

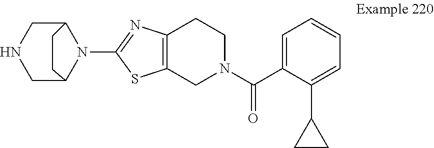 [SMILES: O=C(c1ccccc1C1CC1)N1CCc2nc(N3C4CCC3CNC4)sc2C1]

Prepared using 2-cyclopropylbenzoic acid as the acid component.

¹H-NMR: δ_{H} (400 MHz, CDCl₃, mixture of rotamers) 7.33-7.27 (1H, m), 7.23-7.16 (2H, m), 6.89 (1H, t, J 8.2), 4.92 (0.6H, m), 4.76 (0.67H, m), 4.41-4.33 (0.4H, m), 4.32 (0.1H, s), 4.30-4.24 (0.7H, m), 4.23 (0.1H, s), 4.12 (2H, d, J 13.6), 3.89-3.80 (0.29H, m), 3.56 (1.2H, t, J 5.8), 3.27-3.17 (2H, m), 2.86-2.79 (0.7H, m), 2.71-2.54 (3.3H, m), 2.12-1.99 (2H, m), 1.98-1.81 (3H, m), 1.02-0.71 (3.2H, m), 0.68-0.60 (0.6H, m), 0.50-0.42 (0.4H, m); UPLC (Method A): t_{R} 2.28 min, 99%, MS (ESI) 395.2 (M+H)⁺.

### Example 221: Synthesis of (2-(3,8-diazabicyclo[3.2.1]octan-8-yl)-6,7-dihydrothiazolo[5,4-c]pyridin-5(4H)-yl)(6-chloropyridin-3-yl)methanone

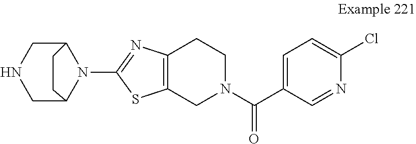 [SMILES: O=C(c1ccc(Cl)nc1)N1CCc2nc(N3C4CCC3CNC4)sc2C1]

Prepared using 6-chloronicotinic acid as the acid component.

¹H-NMR: δ_{H} (400 MHz, CDCl₃, mixture of rotamers) 8.54-8.47 (1H, m), 7.78 (1H, dd, J 8.2, 2.4), 7.43 (1H, d, J 8.1), 4.75 (1.17H, s), 4.47 (0.71H, s), 4.13 (2H, s), 4.04 (0.72H, s), 3.69 (1.21H, s), 3.22 (2H, d, J 12.2), 2.88-2.70 (2H, m), 2.70-2.62 (2H, m), 2.13-1.98 (2H, m), 1.98-1.90 (2H, m); UPLC (Method A): t_{R} 1.61 min, 99%, MS (ESI) 390.2 (M+H)⁺.

### Example 222: Synthesis of (2-(3,8-diazabicyclo[3.2.1]octan-8-yl)-6,7-dihydrothiazolo[5,4-c]pyridin-5(4H)-yl)(4-fluoro-2-methoxyphenyl)methanone

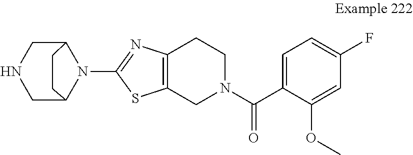 [SMILES: COc1cc(F)ccc1C(=O)N1CCc2nc(N3C4CCC3CNC4)sc2C1]

Prepared using 4-fluoro-2-methoxybenzoic acid as the acid component.

¹H-NMR: δ_{H} (400 MHz, CDCl₃, mixture of rotamers) 7.29-7.21 (1H, m), 6.76-6.61 (2H, m), 4.78 (1H, t, J2.6), 4.40-4.18 (1H, m), 4.17-4.08 (2H, m), 3.81 (3.5H, d, J 16.2), 3.53 (1.5H, m), 3.22 (2H, t, J 11.9), 2.88-2.52 (4H, m), 2.12-1.99 (2H, m), 1.95 (2H, m); UPLC (Method A): t_{R} 1.92 min, 100%, MS (ESI) 403.2 (M+H)⁺.

### Example 223: Synthesis of (2-(3,8-diazabicyclo[3.2.1]octan-8-yl)-6,7-dihydrothiazolo[5,4-c]pyridin-5(4H)-yl)(2-(1,1-difluoroethyl)phenyl)methanone

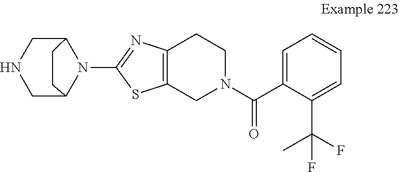 [SMILES: CC(F)(F)c1ccccc1C(=O)N1CCc2nc(N3C4CCC3CNC4)sc2C1]

Prepared using 2-(1,1-difluoroethyl)benzoic acid as the acid component.

¹H-NMR: δ_{H} (400 MHz, CDCl₃, mixture of rotamers) 7.58-7.50 (1H, m), 7.50-7.41 (2H, m), 7.29-7.20 (1H, m), 5.07-4.45 (1H, m), 4.26-3.97 (3.5H, m), 3.63-3.32 (1.5H, m), 3.31-3.15 (2H, m), 2.85-2.56 (4H, m), 2.15-1.86 (7H, m); UPLC (Method A): t_{R} 2.40 min, 100%, MS (ESI) 419.2 (M+H)⁺.

### Example 224: Synthesis of (2-(3,8-diazabicyclo[3.2.1]octan-8-yl)-6,7-dihydrothiazolo[5,4-c]pyridin-5(4H)-yl)(2-chloropyridin-3-yl)methanone

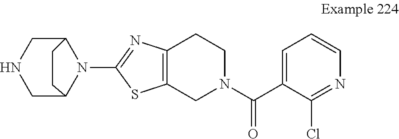 [SMILES: O=C(c1cccnc1Cl)N1CCc2nc(N3C4CCC3CNC4)sc2C1]

Prepared using 2-chloronicotinic acid as the acid component.

¹H-NMR: δ_{H} (400 MHz, CDCl₃, mixture of rotamers) 8.53-8.39 (1H, m), 7.76-7.62 (1H, m), 7.43-7.30 (1H, m), 5.00-4.64 (1H, m), 4.46-4.10 (3H, m), 4.09-3.46 (2H, m), 3.28-3.16 (2H, m), 2.88-2.56 (4H, m), 2.12-1.89 (4H, m); UPLC (Method A): t_{R} 1.42 min, 100%, MS (ESI) 390.4 (M+H)⁺.

### Example 225: Synthesis of (2-(3,8-diazabicyclo[3.2.1]octan-8-yl)-6,7-dihydrothiazolo[5,4-c]pyridin-5(4H)-yl)(2,4-dimethylphenyl)methanone

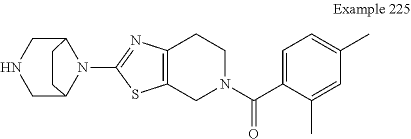 [SMILES: Cc1ccc(C(=O)N2CCc3nc(N4C5CCC4CNC5)sc3C2)c(C)c1]

Prepared using 2,4-dimethylbenzoic acid as the acid component.

¹H-NMR: δ_{H} (400 MHz, CDCl₃, mixture of rotamers) 7.11-6.99 (3H, m), 4.92 (0.6H, s), 4.69 (0.6H, s), 4.24 (1H, s), 4.17-4.08 (2H, m), 3.88 (0.4H, s), 3.53 (1.3H, s), 3.27-3.17 (2H, m), 2.81 (0.8H, s), 2.71-2.52 (3.3H, m), 2.34 (3H, d, J 2.4), 2.29 (2H, s), 2.22 (1H, s), 2.12-1.99 (2H, m), 1.94 (2H, q, J 6.3); UPLC (Method A): t_{R} 2.34 min, 100%, MS (ESI) 383.4 (M+H)⁺.

### Example 226: Synthesis of (2-(3,8-diazabicyclo[3.2.1]octan-8-yl)-6,7-dihydrothiazolo[5,4-c]pyridin-5(4H)-yl)(4-chlorophenyl)methanone

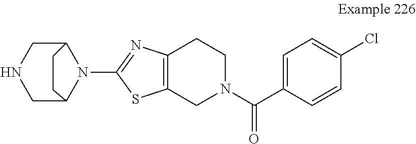 [SMILES: O=C(c1ccc(Cl)cc1)N1CCc2nc(N3C4CCC3CNC4)sc2C1]

Prepared using 4-chlorobenzoic acid as the acid component.

¹H-NMR: δ_{H} (400 MHz, CDCl₃, mixture of rotamers) 7.46-7.37 (4H, m), 4.90-4.07 (4H, m), 4.07-3.11 (4H, m), 2.91-2.50 (4H, m), 2.19-1.90 (4H, m); UPLC (Method A): t_{R} 1.51 min, 99%, MS (ESI) 389.2 (M+H)⁺.

### Example 227: Synthesis of (2-(3,8-diazabicyclo[3.2.1]octan-8-yl)-6,7-dihydrothiazolo[5,4-c]pyridin-5(4H)-yl)(4-(trifluoromethyl)phenyl)methanone

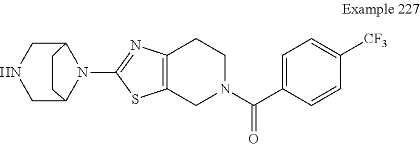 [SMILES: O=C(c1ccc(C(F)(F)F)cc1)N1CCc2nc(N3C4CCC3CNC4)sc2C1]

Prepared using 4-(trifluoromethyl)benzoic acid as the acid component.

¹H-NMR: δ_{H} (400 MHz, CDCl₃, mixture of rotamers) 7.75-7.67 (2H, m), 7.59-7.55 (2H, m), 4.98-4.09 (4H, m), 4.09-3.18 (4H, m), 2.90-2.65 (4H, m), 2.18-1.92 (4H, m); UPLC (Method A): t_{R} 1.64 min, 99%, MS (ESI) 423.2 (M+H)⁺.

### Example 228: Synthesis of (2-(3,8-diazabicyclo[3.2.1]octan-8-yl)-6,7-dihydrothiazolo[5,4-c]pyridin-5(4H)-yl)(4-cyclopropylphenyl)methanone

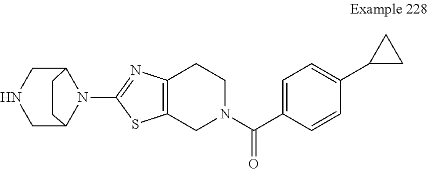 [SMILES: O=C(c1ccc(C2CC2)cc1)N1CCc2nc(N3C4CCC3CNC4)sc2C1]

Prepared using 4-cyclopropylbenzoic acid as the acid component.

¹H-NMR: δ_{H} (400 MHz, CDCl₃, mixture of rotamers) 7.38-7.32 (2H, m), 7.14-7.07 (2H, m), 4.86-4.09 (4H, m), 4.06-3.18 (4H, m), 2.85-2.60 (4H, m), 2.10-1.88 (5H, m), 1.05-0.98 (2H), 0.74 (2H, dt, J 6.7, 4.7); UPLC (Method A): t_{R} 1.64 min, 99%, MS (ESI) 395.2 (M+H)⁺.

### Example 229: Synthesis of (2-(3,8-diazabicyclo[3.2.1]octan-8-yl)-6,7-dihydrothiazolo[5,4-c]pyridin-5(4H)-yl)(4-(difluoromethyl)phenyl)methanone

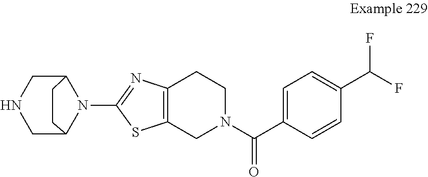 [SMILES: O=C(c1ccc(C(F)F)cc1)N1CCc2nc(N3C4CCC3CNC4)sc2C1]

Prepared using 4-(difluoromethyl)benzoic acid as the acid component.

¹H-NMR: δ_{H} (400 MHz, CDCl₃, mixture of rotamers) 7.63-7.50 (4H, m), 6.68 (1H, t, J 56.2), 4.84-4.09 (4H, m), 4.09-3.14 (4H, m), 2.92-2.60 (4H, m), 2.16-1.83 (4H, m); UPLC (Method A): t_{R} 1.47 min, 99%, MS (ESI) 405.2 (M+H)⁺.

### Example 230: Synthesis of (2-(3,8-diazabicyclo[3.2.1]octan-8-yl)-6,7-dihydrothiazolo[5,4-c]pyridin-5(4H)-yl)(2,3-dihydrobenzofuran-7-yl)methanone

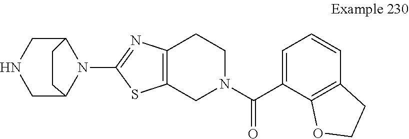 [SMILES: O=C(c1cccc2c1OCC2)N1CCc2nc(N3C4CCC3CNC4)sc2C1]

Prepared using 2,3-dihydrobenzofuran-7-carboxylic acid as the acid component.

¹H-NMR: δ_{H} (400 MHz, CDCl₃, mixture of rotamers) 7.28-7.16 (2H, m), 6.90 (1H, t, J 7.3), 4.77 (1.25H, s), 4.61 (2H, t, J8.7), 4.43 (0.5H, s), 4.14 (2H, s), 3.64 (1.25H, t, J5.7), 3.23 (4H, m), 2.85-2.62 (4H, m), 2.10-2.01 (2H, m), 1.98-1.89 (2H, m); UPLC (Method A): t_{R} 1.96 min, 97%, MS (ESI) 397.2 (M+H)⁺.

### Example 231: Synthesis of (2-(3,8-diazabicyclo[3.2.1]octan-8-yl)-6,7-dihydrothiazolo[5,4-c]pyridin-5(4H)-yl)(4-(2,2-difluoroethyl)phenyl)methanone

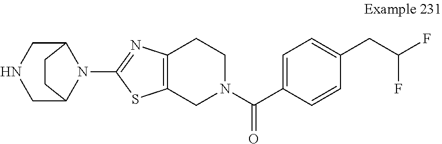 [SMILES: O=C(c1ccc(CC(F)F)cc1)N1CCc2nc(N3C4CCC3CNC4)sc2C1]

Prepared using 4-(2,2-difluoroethyl)benzoic acid as the acid component.

¹H-NMR: δ_{H} (400 MHz, CDCl₃, mixture of rotamers) 7.47-7.40 (2H, m), 7.32 (2H, d, J 7.8), 6.11-5.78 (1H, m), 4.84-4.35 (2H, m), 4.13 (2H, s), 4.08-3.62 (2H, m), 3.28-3.11 (4H, m), 2.89-2.57 (4H, m), 2.12-1.89 (4H, m); UPLC (Method A): t_{R} 1.53 min, 97%, MS (ESI) 419.2 (M+H)⁺.

### Example 232: Synthesis of (2-(3,8-diazabicyclo[3.2.1]octan-8-yl)-6,7-dihydrothiazolo[5,4-c]pyridin-5(4H)-yl)(4-(2,2,2-trifluoroethyl)phenyl)methanone

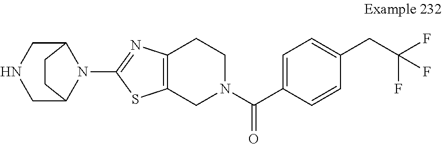 [SMILES: O=C(c1ccc(CC(F)(F)F)cc1)N1CCc2nc(N3C4CCC3CNC4)sc2C1]

Prepared using 4-(2,2,2-trifluoroethyl)benzoic acid as the acid component.

¹H-NMR: δ_{H} (400 MHz, CDCl₃, mixture of rotamers) 7.49-7.42 (2H, m), 7.37 (2H, d, J 7.8), 4.80-4.38 (2H, m), 4.13 (2H, s), 4.08-3.57 (2H, m), 3.41 (2H, m), 3.23 (2H, d, J 12.2), 2.90-2.62 (4H, m), 2.12-1.89 (4H, m); UPLC (Method A): t_{R} 1.64 min, 99%, MS (ESI) 437.2 (M+H)⁺.

### Example 233: Synthesis of (2-(3,8-diazabicyclo[3.2.1]octan-8-yl)-6,7-dihydrothiazolo[5,4-c]pyridin-5(4H)-yl)(2,2-difluorobenzo[d][1,3]dioxol-4-yl)methanone

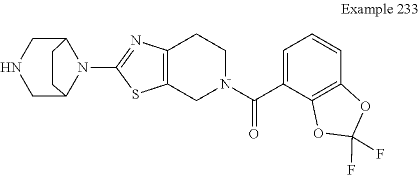 [SMILES: O=C(c1cccc2c1OC(F)(F)O2)N1CCc2nc(N3C4CCC3CNC4)sc2C1]

Prepared using 2,2-difluorobenzo[d][1,3]dioxole-4-carboxylic acid as the acid component.

¹H-NMR: δ_{H} (400 MHz, CDCl₃, mixture of rotamers) 7.24-7.11 (3H, m), 4.78 (1H, t, J 2.0), 4.46 (1H, s), 4.18-4.03 (3H, m), 3.67 (1H, t, J 5.8), 3.22 (2H, t, J 12.5), 2.87-2.73 (2H, m), 2.72-2.62 (2H, m), 2.12-1.89 (4H, m); UPLC (Method A): t_{R} 1.63 min, 99%, MS (ESI) 435.2 (M+H)⁺.

### Example 234: Synthesis of (2-(3,8-diazabicyclo[3.2.1]octan-8-yl)-6,7-dihydrothiazolo[5,4-c]pyridin-5(4H)-yl)(benzo[d][1,3]dioxol-4-yl)methanone

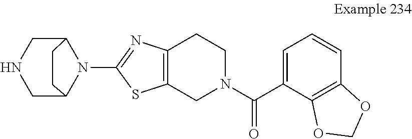 [SMILES: O=C(c1cccc2c1OCO2)N1CCc2nc(N3C4CCC3CNC4)sc2C1]

Prepared using 1,3-benzodioxole-4-carboxylic acid as the acid component.

¹H-NMR: δ_{H} (400 MHz, CDCl₃, mixture of rotamers) 6.99-6.80 (3H, m), 6.01 (2H, s), 4.87-4.43 (2H, m), 4.14 (2H, s), 4.07-3.63 (2H, m), 3.30-3.17 (2H, m), 2.88-2.60 (4H, m), 2.16-1.91 (4H, m); UPLC (Method A): t_{R} 1.38 min, 99%, MS (ESI) 399.2 (M+H)⁺.

### Example 235: Synthesis of (2-(3,8-diazabicyclo[3.2.1]octan-8-yl)-6,7-dihydrothiazolo[5,4-c]pyridin-5(4H)-yl)(3-fluorophenyl)methanone

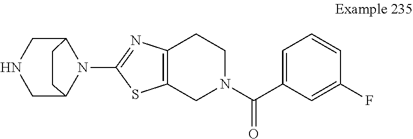 [SMILES: O=C(c1cccc(F)c1)N1CCc2nc(N3C4CCC3CNC4)sc2C1]

Prepared using 3-fluorobenzoic acid as the acid component.

¹H-NMR: δ_{H} (400 MHz, CDCl₃, mixture of rotamers) 7.48-7.35 (1H, m), 7.26-7.20 (3H, m), 7.19-7.10 (2H, m), 4.88-4.36 (2H, m), 4.15 (2H, s), 4.08-3.57 (2H, m), 3.26 (2H, d, J 12.3), 2.89-2.60 (4H, m), 2.22-1.89 (5H, m); UPLC (Method A): t_{R} 1.38 min, 98%, MS (ESI) 373.0 (M+H)⁺.

### Example 236: Synthesis of (2-(3,8-diazabicyclo[3.2.1]octan-8-yl)-6,7-dihydrothiazolo[5,4-c]pyridin-5(4H)-yl)(2,3-dihydrobenzofuran-4-yl)methanone

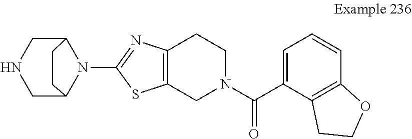 [SMILES: O=C(c1cccc2c1CCO2)N1CCc2nc(N3C4CCC3CNC4)sc2C1]

Prepared using 2,3-dihydrobenzofuran-4-carboxylic acid as the acid component.

¹H-NMR: δ_{H} (400 MHz, CDCl₃, mixture of rotamers) 7.16 (1H, t, J 7.8), 6.88-6.75 (2H, m), 4.84-4.34 (4H, m), 4.18 (2H, s), 4.12-3.60 (2H, m), 3.36-3.13 (4H, m), 2.87-2.58 (4H, m), 2.18-2.02 (5H, m); UPLC (Method A): t_{R} 1.29 min, 98%, MS (ESI) 397.2 (M+H)⁺.

### Example 237: Synthesis of (2-(3,8-diazabicyclo[3.2.1]octan-8-yl)-6,7-dihydrothiazolo[5,4-c]pyridin-5(4H)-yl)(quinolin-8-yl)methanone

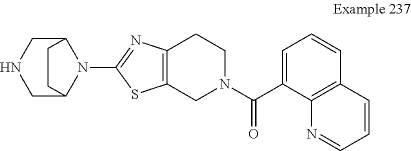 [SMILES: O=C(c1cccc2cccnc12)N1CCc2nc(N3C4CCC3CNC4)sc2C1]

Prepared using quinoline-8-carboxylic acid as the acid component.

¹H-NMR: δ_{H} (400 MHz, CDCl₃, mixture of rotamers) 8.98-8.91 (1H, m), 8.20 (1H, dt, J 8.4, 1.5), 7.92-7.85 (1H, m), 7.72 (1H, dt, J 7.0, 1.5), 7.64-7.55 (1H, m), 7.51-7.41 (1H, m), 5.05-4.76 (2H, m), 4.32-4.05 (2H, m), 3.77-3.39 (2H, m), 3.35-3.20 (2H, m), 2.93-2.46 (4H, m), 2.19-1.97 (4H, m); UPLC (Method A): t_{R} 1.27 min, 99%, MS (ESI) 406.2 (M+H)⁺.

### Example 238: Synthesis of 2-(2-(3,8-diazabicyclo[3.2.1]octan-8-yl)-4,5,6,7-tetrahydrothiazolo[5,4-c]pyridine-5-carbonyl)-5-methylbenzonitrile

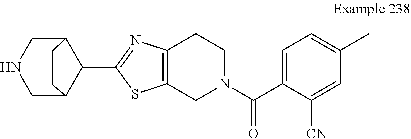 [SMILES: Cc1ccc(C(=O)N2CCc3nc(C4C5CCC4CNC5)sc3C2)c(C#N)c1]

Prepared using 2-cyano-4-methylbenzoic acid as the acid component.

¹H-NMR: δ_{H} (400 MHz, CDCl₃, mixture of rotamers) 7.59-7.50 (1H, m), 7.50-7.41 (1H, m), 7.41-7.30 (1H, m), 4.98-4.27 (2H, m), 4.24-3.97 (3H, m), 3.60 (1H, t, J 5.8), 3.32-3.10 (2H, m), 2.91-2.78 (1H, m), 2.75-2.58 (3H, m), 2.43 (3H, s), 2.17-1.84 (4H, m); UPLC (Method A): t_{R} 1.99 min, 95%, MS (ESI) 394.2 (M+H)⁺.

### Example 239: Synthesis of 4-(2-(3,8-diazabicyclo[3.2.1]octan-8-yl)-4,5,6,7-tetrahydrothiazolo[5,4-c]pyridine-5-carbonyl)benzonitrile

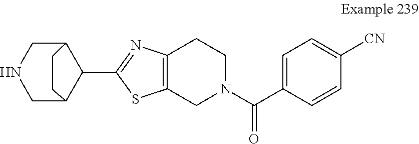 [SMILES: N#Cc1ccc(C(=O)N2CCc3nc(C4C5CCC4CNC5)sc3C2)cc1]

Prepared using 4-cyanobenzoic acid as the acid component.

¹H-NMR: δ_{H} (400 MHz, CDCl₃, mixture of rotamers) 7.80-7.69 (2H, m), 7.61-7.48 (2H, m), 4.83-4.08 (4H, m), 4.09-3.14 (4H, m), 2.86-2.60 (4H, m), 2.13-1.87 (4H, m); UPLC (Method A): t_{R} 1.31 min, 96%, MS (ESI) 380.0 (M+H)⁺.

### Example 240: Synthesis of (2-(3,8-diazabicyclo[3.2.1]octan-8-yl)-6,7-dihydrothiazolo[5,4-c]pyridin-5(4H)-yl)(6-fluoro-2-methoxypyridin-3-yl)methanone

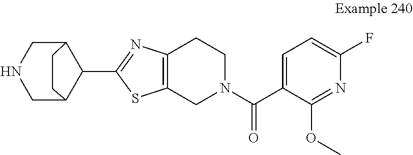 [SMILES: COc1nc(F)ccc1C(=O)N1CCc2nc(C3C4CCC3CNC4)sc2C1]

Prepared using 6-fluoro-2-methoxynicotinic acid as the acid component.

¹H-NMR: δ_{H} (400 MHz, CDCl₃, mixture of rotamers) 7.79-7.70 (1H, m), 6.59-6.52 (1H, m), 4.84-4.08 (4H, m), 3.99-3.92 (3H, m), 3.75-3.15 (4H, m), 2.86-2.60 (4H, m), 2.09-1.91 (4H, m); UPLC (Method D): t_{R} 2.80 min, 95%, MS (ESI) 404.2 (M+H)⁺.

### Example 241: Synthesis of (2-(3,8-diazabicyclo[3.2.1]octan-8-yl)-6,7-dihydrothiazolo[5,4-c]pyridin-5(4H)-yl)(6-(trifluoromethyl)pyridin-3-yl)methanone

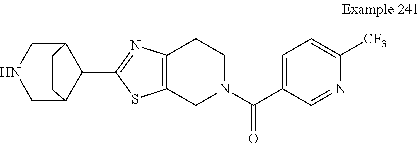 [SMILES: O=C(c1ccc(C(F)(F)F)nc1)N1CCc2nc(C3C4CCC3CNC4)sc2C1]

Prepared using 6-(trifluoromethyl)nicotinic acid as the acid component.

¹H-NMR: δ_{H} (400 MHz, CDCl₃, mixture of rotamers) 8.82 (1H, s), 7.99 (1H, dd, J 8.0), 7.79 (1H, d, J 7.8), 4.87-4.40 (2H, m), 4.18-3.62 (4H, m), 3.24 (2H, d, J 12.5), 2.89-2.63 (4H, m), 2.14-1.91 (4H, m); UPLC (Method A): t_{R} 1.38 min, 100%, MS (ESI) 424.0 (M+H)⁺.

### Example 242: Synthesis of (2-(3,8-diazabicyclo[3.2.1]octan-8-yl)-6,7-dihydrothiazolo[5,4-c]pyridin-5(4H)-yl)(6-fluoro-4-methylpyridin-3-yl)methanone

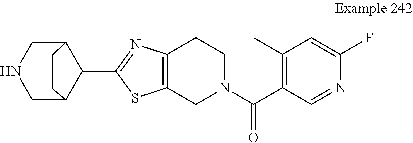 [SMILES: Cc1cc(F)ncc1C(=O)N1CCc2nc(C3C4CCC3CNC4)sc2C1]

Prepared using 6-fluoro-4-methylnicotinic acid as the acid component.

¹H-NMR: δ_{H} (400 MHz, CDCl₃, mixture of rotamers) 8.09 (1H, d, J 10.3), 6.85 (1H, d, J 12.8), 5.01-4.19 (4H, m), 3.62-3.54 (1H, m), 3.49-3.37 (2H, m), 3.14-3.02 (2H, m), 2.90-2.60 (2H, m), 2.42-1.98 (8H, m); UPLC (Method A): t_{R} 1.47 min, 99%, MS (ESI) 388.2 (M+H)⁺.

### Example 243: Synthesis of (2-(3,8-diazabicyclo[3.2.1]octan-8-yl)-6,7-dihydrothiazolo[5,4-c]pyridin-5(4H)-yl)(6-(difluoromethyl)pyridin-3-yl)methanone

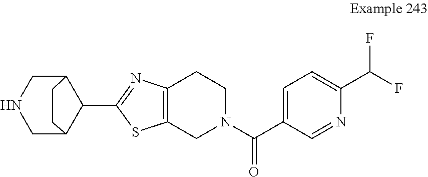 [SMILES: O=C(c1ccc(C(F)F)nc1)N1CCc2nc(C3C4CCC3CNC4)sc2C1]

Prepared using 6-(difluoromethyl)nicotinic acid as the acid component.

¹H-NMR: δ_{H} (400 MHz, CDCl₃, mixture of rotamers) 8.75 (1H, s), 7.95 (1H, dd, J8.0, 2.1), 7.74 (1H, d, J 7.8), 6.86-6.49 (1H, m), 4.87-4.38 (2H, m), 4.19-3.61 (4H, m), 3.30-3.16 (2H, m), 2.91-2.63 (4H, m), 2.13-1.89 (4H, m); UPLC (Method A): t_{R} 1.49 min, 98%, MS (ESI) 406.2 (M+H)⁺.

### Example 244: Synthesis of cyclopentyl 2-(3,8-diazabicyclo[3.2.1]octan-8-yl)-6,7-dihydrothiazolo[5,4-c]pyridine-5(4H)-carboxylate

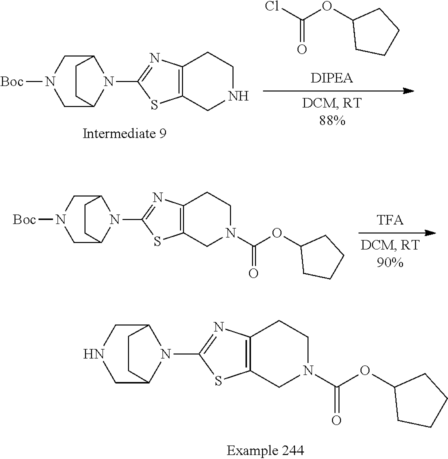 [SMILES: CC(C)(C)OC(=O)N1CC2CCC(C1)N2c1nc2c(s1)CNCC2 ; O=C(Cl)OC1CCCC1 ; CC(C)(C)OC(=O)N1CC2CCC(C1)N2c1nc2c(s1)CN(C(=O)OC1CCCC1)CC2 ; O=C(OC1CCCC1)N1CCc2nc(N3C4CCC3CNC4)sc2C1]

To a mixture of tert-butyl 8-(4,5,6,7-tetrahydrothiazolo[5,4-c]pyridin-2-yl)-3,8-diazabicyclo[3.2.1]octane-3-carboxylate (81 mg, 0.23 mmol) and diisopropylethylamine (0.15 mL, 0.90 mmol) in dichloromethane (2 mL) was added cyclopentyl chloroformate (0.11 mL, 0.90 mmol) and reaction mixture was stirred at room temperature for 1 hour.

The mixture was concentrated in vacuo and purified with silica column chromatography (0% to 100% ethyl acetate in n-heptane) to afford cyclopentyl 2-(3-(tert-butoxycarbonyl)-3,8-diazabicyclo[3.2.1]octan-8-yl)-6,7-dihydrothiazolo[5,4-c]pyridine-5(4H)-carboxylate (96 mg, 88%) as a white solid. LCMS (Method B): t_{R} 2.26 min, 98%, MS (ESI) 463.2 (M+H)⁺.

To a solution of cyclopentyl 2-(3-(tert-butoxycarbonyl)-3,8-diazabicyclo[3.2.1]octan-8-yl)-6,7-dihydrothiazolo[5,4-c]pyridine-5(4H)-carboxylate (95 mg, 0.21 mmol) in dichloromethane (1 mL) was added trifluoroacetic acid (0.32 mL, 4.11 mmol) and the mixture was stirred at room temperature for 1 hour. The mixture was concentrated in vacuo, partitioned between saturated aqueous potassium carbonate and dichlormethane and the layers were separated. The aqueous layer was extracted with dichloromethane three times, dried with sodium sulfate and concentrated in vacuo. The residue was lyophilized to afford cyclopentyl 2-(3,8-diazabicyclo[3.2.1]octan-8-yl)-6,7-dihydrothiazolo[5,4-c]pyridine-5(4H)-carboxylate (67 mg, 90%, Example 244) as an off-white solid. ¹H-NMR: δ_{H} (400 MHz, CDCl₃, mixture of rotamers) 5.20-5.09 (1H, m), 4.58-4.37 (2H, m), 4.19-4.06 (2H, m), 3.84-3.61 (2H, m), 3.21 (2H, dd, J 12.4, 1.8), 2.65 (4H, dd, J 12.5, 2.3), 2.13-1.97 (2H, m), 1.97-1.79 (4H, m), 1.79-1.65 (4H, m), 1.65-1.61 (1H, m), 1.60-1.55 (1H, m); UPLC (Method A): t_{R} 1.61 min, 99%, MS (ESI) 363.2 (M+H)⁺.

The following examples 245-247) were prepared using procedures analogous to Example 244, using the appropriate starting materials.

### Example 245: Synthesis of isopropyl 2-(3,8-diazabicyclo[3.2.1]octan-8-yl)-6,7-dihydrothiazolo[5,4-c]pyridine-5(4H)-carboxylate

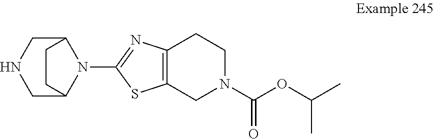 [SMILES: CC(C)OC(=O)N1CCc2nc(N3C4CCC3CNC4)sc2C1]

Prepared using isopropyl chloroformate as the starting reagent.

¹H-NMR: δ_{H} (400 MHz, CDCl₃, mixture of rotamers) 4.96 (1H, hept, J 6.3), 4.48 (2H, s), 4.13 (2H, dd, J 4.5, 2.3), 3.74 (2H, s), 3.22 (2H, dd, J 12.5, 1.8), 2.72-2.62 (4H, m), 2.09-2.01 (2H, m), 1.97-1.88 (2H, m), 1.26 (6H, d, J 6.2); UPLC (Method A): t_{R} 1.41 min, 93%, MS (ESI) 337.2 (M+H)⁺.

### Example 246: Synthesis of benzyl 2-(3,8-diazabicyclo[3.2.1]octan-8-yl)-6,7-dihydrothiazolo[5,4-c]pyridine-5(4H)-carboxylate

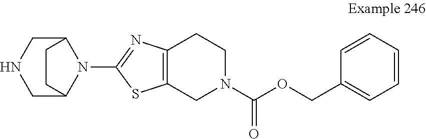 [SMILES: O=C(OCc1ccccc1)N1CCc2nc(N3C4CCC3CNC4)sc2C1]

Prepared using benzyl chloroformate as the starting reagent.

¹H-NMR: δ_{H} (400 MHz, CDCl₃, mixture of rotamers) 7.39-7.29 (5H, m), 5.17 (2H, s), 4.53 (2H, s), 4.12 (2H, m), 3.79 (2H, s), 3.22 (2H, dd, J 12.3, 1.7), 2.66 (4H, dd, J 12.5, 2.2), 2.10-2.00 (2H, m), 2.00-1.88 (2H, m); UPLC (Method A): t_{R} 2.61 min, 99%, MS (ESI) 385.2 (M+H)⁺.

### Example 247: Synthesis of phenyl 2-(3,8-diazabicyclo[3.2.1]octan-8-yl)-6,7-dihydrothiazolo[5,4-c]pyridine-5(4H)-carboxylate

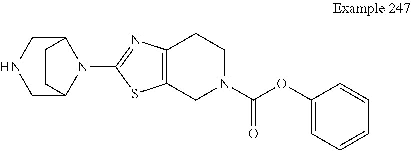 [SMILES: O=C(Oc1ccccc1)N1CCc2nc(N3C4CCC3CNC4)sc2C1]

Prepared using phenyl chloroformate as the starting reagent.

¹H-NMR: δ_{H} (400 MHz, CDCl₃, mixture of rotamers) 7.40-7.32 (2H, m), 7.24-7.17 (1H, m), 7.16-7.09 (2H, m), 4.71 (1H, s), 4.60 (1H, s), 4.15 (2H, s), 3.94 (1H, t, J 6.0), 3.86 (1H, t), 3.24 (2H, dd, J 12.1, 1.9), 2.79 (2H, s), 2.68 (2H, dd, J 12.2, 2.3), 2.10-1.99 (2H, m), 1.95 (2H, dd, J 7.6, 5.5); UPLC (Method A): t_{R} 2.34 min, 99%, MS (ESI) 371.2 (M+H)⁺.

### Example 248: Synthesis of 1-(2-(3,8-diazabicyclo[3.2.1]octan-8-yl)-6,7-dihydrothiazolo[5,4-c]pyridin-5(4H)-yl)-2-hydroxy-2-(1-methylcyclopentyl)ethan-1-one

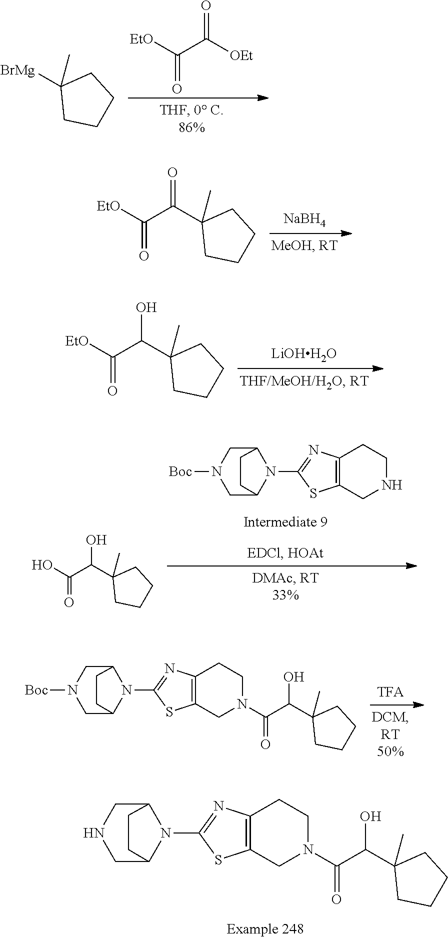

To a solution of diethyl oxalate (3.97 mL, 29.4 mmol) in tetrahydrofuran (10 mL) at 0° C. was added (1-methylcyclopentyl) magnesium bromide (1.1 g, 5.87 mmol) and the mixture was stirred at room temperature for 2 hours. The mixture was quenched with saturated ammonium chloride solution and was extracted with ethyl acetate twice. The combined organic layers were dried with sodium sulfate and concentrated to afford ethyl 2-(1-methylcyclopentyl)-2-oxoacetate as a yellow oil. GCMS: t_{R} 2.72 min, 99%, MS (ESI) 184.1 (M). This was taken forward into the next step without further purification.

To a solution of ethyl 2-(1-methylcyclopentyl)-2-oxoacetate (1 g, 5.43 mmol) in methanol (10 mL) was added sodium borohydride (0.31 g, 8.14 mmol) and the mixture was stirred at room temperature for 16 hours. The mixture was concentrated in vacuo, the residue was coated onto silica and purified with silica column chromatography (0% to 100% ethyl acetate in n-heptane) to afford ethyl 2-hydroxy-2-(1-methylcyclopentyl)acetate (260 mg, 25%) as a colorless oil. ¹H-NMR: δ_{H} (400 MHz, CDCl₃, mixture of rotamers) 4.41-4.17 (2H, m), 3.91 (1H, d, J 6.1), 2.90 (1H, d, J 6.6), 1.82-1.59 (6H, m), 1.45-1.33 (2H, m), 1.33-1.29 (3H, m), 0.91 (3H, s). This was taken forward into the next step without further purification.

A solution of ethyl 2-hydroxy-2-(1-methylcyclopentyl)acetate (260 mg, 1.40 mmol) and lithium hydroxide monohydrate (176 mg, 4.19 mmol) in tetrahydrofuran (5 mL) and water (2 mL) was stirred at room temperature for 20 hours. The mixture was acidified with 1M hydrochloric acid solution to pH ˜3 and concentrated to afford 2-hydroxy-2-(1-methylcyclopentyl)acetic acid (221 mg, quant.) as a white solid. This was taken forward into the next step without further purification.

A solution of tert-butyl 8-(4,5,6,7-tetrahydrothiazolo[5,4-c]pyridin-2-yl)-3,8-diazabicyclo[3.2.1]octane-3-carboxylate (300 mg, 0.86 mmol, Intermediate 9), 2-hydroxy-2-(1-methylcyclopentyl)acetic acid (220 mg, 1.39 mmol), N-(3-dimethylaminopropyl)-N′-ethylcarbodiimide hydrochloride (328 mg, 1.71 mmol) and 1-hydroxy-7-azabenzotriazole (11 mg, 0.09 mmol) in N,N-dimethylacetamide (2 mL) was stirred at room temperature for 3 hours. The mixture was purified with preparative reversed phase chromatography (Waters XSelect™ CSH C18 column, ammonium bicarbonate in water/acetonitrile) and lyophilized to afford tert-butyl 8-(5-(2-hydroxy-2-(1-methylcyclopentyl)acetyl)-4,5,6,7-tetrahydrothiazolo[5,4-c]pyridin-2-yl)-3,8-diazabicyclo[3.2.1]octane-3-carboxylate (147 mg, 33%) as a colorless gum. LCMS (Method B): t_{R} 2.11 min, 95%, MS (ESI) 491.3 (M+H)⁺.

To a solution of tert-butyl 8-(5-(2-hydroxy-2-(1-methylcyclopentyl)acetyl)-4,5,6,7-tetrahydrothiazolo[5,4-c]pyridin-2-yl)-3,8-diazabicyclo[3.2.1]octane-3-carboxylate (50 mg, 0.10 mmol) in dichloromethane (2 mL) was added trifluoroacetic acid (0.5 mL, 6.49 mmol) and the mixture was stirred at room temperature for 3 hours. The mixture was concentrated, purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-hydroxy-2-(1-methylcyclopentyl)ethan-1-one (24 mg, 50%, Example 248) as a white solid. ¹H-NMR: δ_{H} (400 MHz, CDCl₃, mixture of rotamers) 4.84-4.47 (2H, m), 4.45-4.29 (1H, m), 4.18-4.07 (2H, m), 4.05-3.72 (2H, m), 3.49-3.28 (1H, m), 3.27-3.18 (2H, m), 2.83-2.60 (4H, m), 2.13-2.02 (2H, m), 2.00-1.89 (2H, m), 1.83-1.63 (6H, m), 1.38-1.24 (2H, m), 1.04-0.91 (3H, m); UPLC (Method A): t_{R} 1.51 min, 97%, MS (ESI) 391.2 (M+H)⁺.

### Example 249: Synthesis of 1-(2-(3,8-diazabicyclo[3.2.1]octan-8-yl)-6,7-dihydrothiazolo[5,4-c]pyridin-5(4H)-yl)-2-methoxy-2-(1-methylcyclopentyl)ethan-1-one

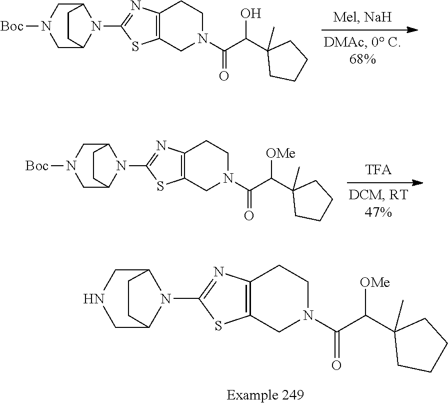 [SMILES: CC(C)(C)OC(=O)N1CC2CCC(C1)N2c1nc2c(s1)CN(C(=O)C(O)C1(C)CCCC1)CC2 ; COC(C(=O)N1CCc2nc(N3C4CCC3CN(C(=O)OC(C)(C)C)C4)sc2C1)C1(C)CCCC1 ; COC(C(=O)N1CCc2nc(N3C4CCC3CNC4)sc2C1)C1(C)CCCC1]

To a solution of tert-butyl 8-(5-(2-hydroxy-2-(1-methylcyclopentyl)acetyl)-4,5,6,7-tetrahydrothiazolo[5,4-c]pyridin-2-yl)-3,8-diazabicyclo[3.2.1]octane-3-carboxylate (50 mg, 0.10 mmol, Example 248) in N,N-dimethylacetamide (2 mL) was added 60% sodium hydride in mineral oil (4.08 mg, 0.10 mmol) and the mixture was stirred at room temperature for 16 hours. The mixture was purified with preparative reversed phase chromatography (Waters XSelect™ CSH C18 column, ammonium bicarbonate in water/acetonitrile) and lyophilized to afford tert-butyl 8-(5-(2-methoxy-2-(1-methylcyclopentyl)acetyl)-4,5,6,7-tetrahydrothiazolo[5,4-c]pyridin-2-yl)-3,8-diazabicyclo[3.2.1]octane-3-carboxylate (35 mg, 68%) as a colorless gum.

To a solution of tert-butyl 8-(5-(2-methoxy-2-(1-methylcyclopentyl)acetyl)-4,5,6,7-tetrahydrothiazolo[5,4-c]pyridin-2-yl)-3,8-diazabicyclo[3.2.1]octane-3-carboxylate (50 mg, 0.10 mmol) in dichloromethane (2 mL) was added trifluoroacetic acid (0.5 mL, 6.49 mmol) and the mixture was stirred at room temperature for 16 hours. The mixture was concentrated, purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-methoxy-2-(1-methylcyclopentyl)ethan-1-one (20 mg, 47%, Example 249) as a white solid. ¹H-NMR: δ_{H} (400 MHz, CDCl₃, mixture of rotamers) 4.93-4.57 (2H, m), 4.17-3.78 (5H, m), 3.39-3.29 (3H, m), 3.27-3.19 (2H, m), 2.81-2.61 (4H, m), 2.12-2.02 (2H, m), 1.99-1.90 (2H, m), 1.86-1.62 (6H, m), 1.43-1.21 (2H, m), 1.08-0.91 (3H, m); UPLC (Method A): t_{R} 1.61 min, 100%, MS (ESI) 405.2 (M+H)⁺.

### Example 250: Synthesis of 1-(2-(3,8-diazabicyclo[3.2.1]octan-8-yl)-6,7-dihydrothiazolo[5,4-c]pyridin-5(4H)-yl)-2-(2-(2-hydroxyethoxy)phenoxy)ethan-1-one

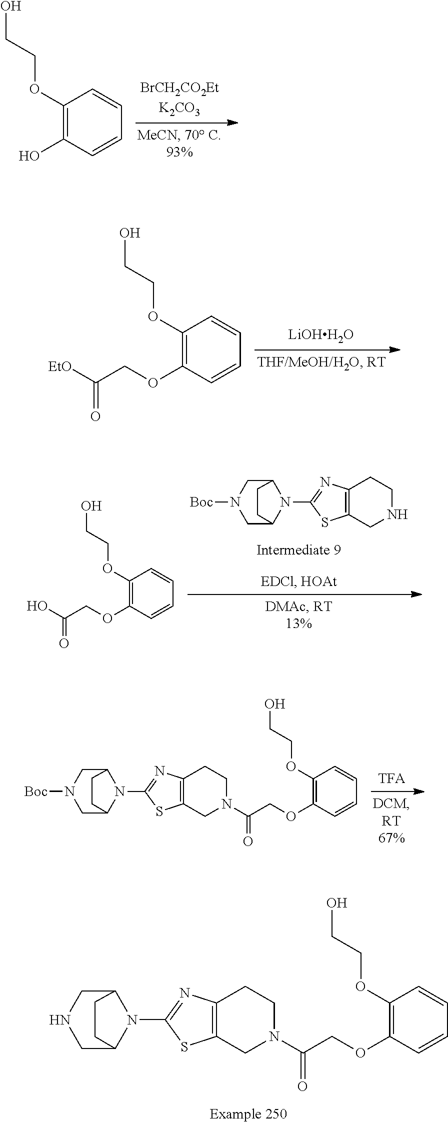

Ethyl bromoacetate (0.36 mL, 3.20 mmol), 2-(2-hydroxyethoxy)phenol (470 mg, 3.05 mmol) and potassium carbonate (463 mg, 3.35 mmol) were suspended in acetonitrile (6 mL) and the mixture was stirred at 70° C. for 16 hours. The mixture was filtered over Celite, thoroughly washed with acetonitrile and concentrated in vacuo to afford ethyl 2-(2-(2-hydroxyethoxy)phenoxy)acetate (756 mg, 93%) as a light yellow oil. LCMS (Method B): t_{R} 1.72 min, 90%, MS (ESI) 241.1 (M+H)⁺.

To a solution of ethyl 2-(2-(2-hydroxyethoxy)phenoxy)acetate (750 mg, 3.12 mmol) in methanol (5 mL) was added lithium hydroxide monohydrate (144 mg, 3.43 mmol) and water (2 mL). The mixture was stirred at room temperature for 1 hour, acidified with 12M HCl (˜5 mL) and concentrated in vacuo to afford 2-(2-(2-hydroxyethoxy)phenoxy)acetic acid (1.04 g, quant.) as a light tan solid. ¹H-NMR: δ_{H} (400 MHz, CDCl₃, mixture of rotamers) 7.06-6.92 (1H, m), 6.92-6.76 (3H, m), 4.54 (2H, s), 3.99 (2H, t, J5.1), 3.71 (2H, t, J5.1); LCMS (Method A): t_{R} 1.30 min, 98%, MS (ESI) 213.0 (M+H)⁺.

A mixture of 2-(2-(2-hydroxyethoxy)phenoxy)acetic acid (97 mg, 0.46 mmol), N-(3-dimethylaminopropyl)-N′-ethylcarbodiimide hydrochloride (88 mg, 0.46 mmol) and 1-hydroxy-7-azabenzotriazole (4 mg, 0.04 mmol) in dichloromethane (3 mL) was stirred at room temperature for 5 minutes. Next, tert-butyl 8-(4,5,6,7-tetrahydrothiazolo[5,4-c]pyridin-2-yl)-3,8-diazabicyclo[3.2.1]octane-3-carboxylate (100 mg, 0.29 mmol, Intermediate 9) was added and the mixture was stirred at room temperature for 20 hours. The mixture was purified with silica column chromatography (0% to 100% ethyl acetate in n-heptane) to afford tert-butyl 8-(5-(2-(2-(2-hydroxyethoxy)phenoxy)acetyl)-4,5,6,7-tetrahydrothiazolo[5,4-c]pyridin-2-yl)-3,8-diazabicyclo[3.2.1]octane-3-carboxylate (27 mg, 13%) as a white solid. LCMS (Method A): t_{R} 1.97 min, 98%, MS (ESI) 545.2 (M+H)⁺.

To a solution of tert-butyl 8-(5-(2-(2-(2-hydroxyethoxy)phenoxy)acetyl)-4,5,6,7-tetrahydrothiazolo[5,4-c]pyridin-2-yl)-3,8-diazabicyclo[3.2.1]octane-3-carboxylate (57 mg, 0.11 mmol) in dichloromethane (1 mL) was added trifluoroacetic acid (0.16 mL, 2.1 mmol) and the mixture was stirred at room temperature for 30 minutes. The mixture was concentrated and redissolved in 7N ammonia in methanol (1 mL). The solution was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-(2-(2-hydroxyethoxy)phenoxy)ethan-1-one (32 mg, 67%, Example 250) as a white solid. ¹H-NMR: δ_{H} (400 MHz, CDCl₃, mixture of rotamers) 7.03-6.86 (4H, m), 4.78 (2H, d, J 2.3), 4.71-4.62 (2H, m), 4.18-4.03 (4H, m), 3.95-3.80 (4H, m), 3.29-3.16 (2H, m), 2.97-2.86 (1.3H, m), 2.77-2.58 (2.7H, m), 2.13-1.99 (2H, m), 1.99-1.89 (2H, m), 1.65 (1H, s); UPLC (Method A): t_{R} 0.67 min, 97%, MS (ESI) 445.3 (M+H)⁺.

### Example 251: Synthesis of 1-(2-(3,8-diazabicyclo[3.2.1]octan-8-yl)-6,7-dihydrothiazolo[5,4-c]pyridin-5(4H)-yl)-2,2-difluoro-2-(2-fluoropyridin-4-yl)ethan-1-one

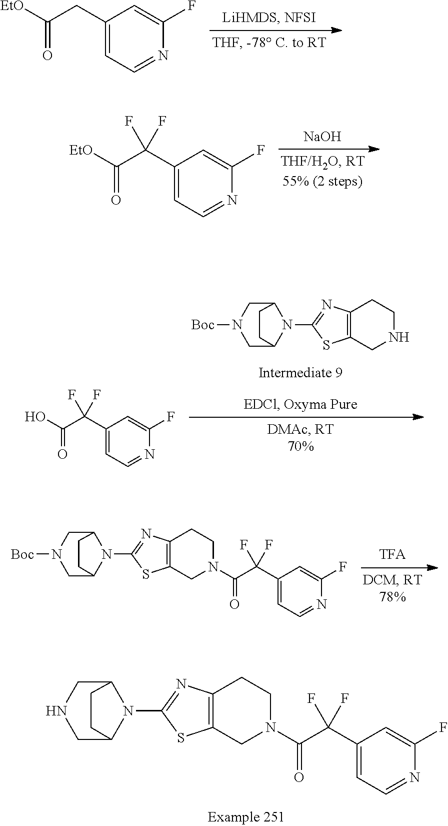

A solution of ethyl 2-(2-fluoropyridin-4-yl)acetate (282 mg, 1.54 mmol) in tetrahydrofuran (2.5 mL) was cooled to −78° C. and 1M lithium bis(trimethylsilyl)amide in tetrahydrofuran (3.5 mL, 3.54 mmol) was added dropwise. The mixture was stirred at −78° C. for 40 minutes and a solution of N-fluorobenzenesulfonimide (1.07 g, 3.39 mmol) in 3.5 mL tetrahydrofuran was added dropwise at −78° C. over 15 minutes. The mixture was allowed to reach room temperature and was stirred for 16 hours. The mixture was quenched with saturated ammonium chloride solution, diluted with diethyl ether and the layers were separated. The organic layer was washed with water and brine, dried over sodium sulfate and partially concentrated in vacuo (at 450 mbar) to afford ethyl 2,2-difluoro-2-(2-fluoropyridin-4-yl)acetate (2.62 g) as an orange liquid/solution. GCMS: t_{R} 2.45 min, 98%, MS (ESI) 146.0 (M). This was taken forward into the next step without further purification

The above solution of ethyl 2,2-difluoro-2-(2-fluoropyridin-4-yl)acetate (2.62 g) was diluted with tetrahydrofuran (10 mL) and water (5 mL). Sodium hydroxide (61 mg, 1.54 mmol) was added and the turbid mixture was stirred at room temperature for 16 hours. The mixture was transferred into a separation funnel and washed with diethyl ether. The layers were separated and the aqueous layer was acidified with concentrated hydrochloric acid to PH ˜3. The aqueous layer was extracted with ethyl acetate twice. The combined organic layers were washed with brine, dried with sodium sulfate and concentrated in vacuo to afford 2,2-difluoro-2-(2-fluoropyridin-4-yl)acetic acid (164 mg, 55% 2 steps) as a yellow solid. LCMS (Method A): t_{R} 1.33 min, 97%, MS (ESI) 192.0 (M+H)⁺.

To a suspension of tert-butyl 8-(4,5,6,7-tetrahydrothiazolo[5,4-c]pyridin-2-yl)-3,8-diazabicyclo[3.2.1]octane-3-carboxylate (76 mg, 0.22 mmol, Intermediate 9) and 2,2-difluoro-2-(2-fluoropyridin-4-yl)acetic acid (41 mg, 0.22 mmol) in N,N-dimethylformamide (1 mL) was added N-(3-dimethylaminopropyl)-N′-ethylcarbodiimide hydrochloride (46 mg, 0.24 mmol) and Oxyma Pure (31 mg, 0.22 mmol). The mixture was stirred at room temperate for 3 days. The mixture was partitioned between ethyl acetate and water, the layers were separated and the aqueous layer was extracted with ethyl acetate. The combined organic layers were washed with brine, dried over sodium sulfate and concentrated in vacuo. The residue was purified with silica column chromatography (10% to 60% ethyl acetate in n-heptane) to afford tert-butyl 8-(5-(2,2-difluoro-2-(2-fluoropyridin-4-yl)acetyl)-4,5,6,7-tetrahydrothiazolo[5,4-c]pyridin-2-yl)-3,8-diazabicyclo[3.2.1]octane-3-carboxylate (80 mg, 70%) as a white solid. LCMS (Method B): t_{R} 2.10 min, 97%, MS (ESI) 524.1 (M−H)⁺.

To a solution of tert-butyl 8-(5-(2,2-difluoro-2-(2-fluoropyridin-4-yl)acetyl)-4,5,6,7-tetrahydrothiazolo[5,4-c]pyridin-2-yl)-3,8-diazabicyclo[3.2.1]octane-3-carboxylate (79 mg, 0.15 mmol) in dichloromethane (2 mL) was added trifluoroacetic acid (0.58 mL, 7.5 mmol) and the mixture was stirred for 15 minutes. The mixture was concentrated in vacuo and coevaporated with dichloromethane The residue was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2,2-difluoro-2-(2-fluoropyridin-4-yl)ethan-1-one (50 mg, 78%, Example 251) as a white solid. ¹H-NMR: δ_{H} (400 MHz, CDCl₃, mixture of rotamers) 8.42-8.29 (1H, m), 7.40-7.29 (1H, m), 7.17-7.05 (1H, m), 4.75-4.55 (2H, m), 4.24-4.04 (2H, m), 4.02-3.84 (2H, m), 3.31-3.11 (2H, m), 2.83-2.59 (4H, m), 2.15-1.85 (4H, m); UPLC (Method A): t_{R} 2.12 min, 99%, MS (ESI) 424.2 (M−H)⁺.

### Example 252: Synthesis of 1-(2-(3,8-diazabicyclo[3.2.1]octan-8-yl)-6,7-dihydrothiazolo[5,4-c]pyridin-5(4H)-yl)-2-(2-(difluoromethoxy)phenoxy)ethan-1-one

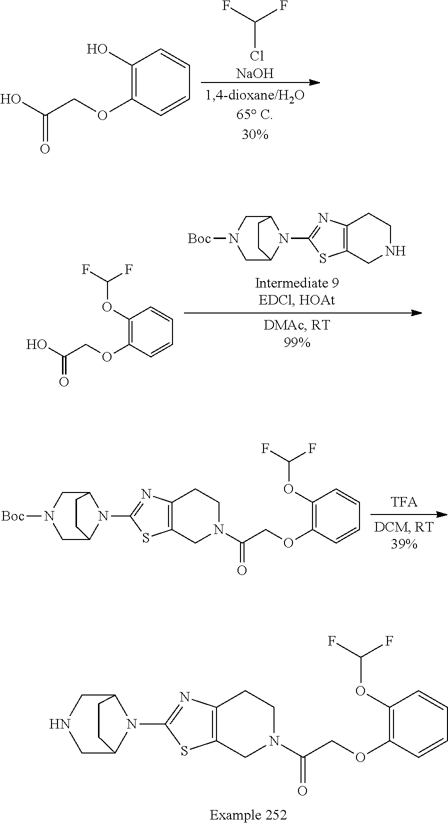

To a solution of 2-hydroxyphenoxyacetic acid (1.13 g, 6.72 mmol) in 1,4-dioxane (5 mL) was added a solution of sodium hydroxide (952 mg, 23.8 mmol) in water (12 mL) and the mixture was heated to 65° C. Next, chlorodifluoromethane was bubbled through via a syringe over the course of 2 hour while stirring at 65° C. The mixture was acidified (pH ˜3) with concentrated hydrochloric acid and left to stand in an ice bath for 10 minutes. The resulting suspension was extracted with ethyl acetate three times, the combined organic layers were filtered and concentrated in vacuo. The residue was purified with preparative reversed phase chromatography (Waters XSelect™ CSH C18 column, ammonium bicarbonate in water/acetonitrile) and lyophilized to afford 2-(2-(difluoromethoxy)phenoxy)acetic acid (448 mg, 30%) as a white solid. ¹H-NMR: δ_{H} (400 MHz, CDCl₃, mixture of rotamers) 9.64 (1H, s), 7.24-7.15 (2H, m), 7.07-6.98 (1H, m), 6.93 (1H, dd, J 8.1, 1.4), 6.84-6.41 (1H, m), 4.75 (2H, s); LCMS (Method A): t_{R} 1.63 min, 99%, MS (ESI) 217.0 (M−H)⁺.

A mixture of 2-(2-(difluoromethoxy)phenoxy)acetic acid (69 mg, 0.32 mmol), N-(3-dimethylaminopropyl)-N-ethylcarbodiimide hydrochloride (74 mg, 0.39 mmol) and 1-hydroxy-7-azabenzotriazole (3 mg, 0.02 mmol) in dichloromethane (3 mL) was stirred at room temperature for 5 minutes. Next, tert-butyl 8-(4,5,6,7-tetrahydrothiazolo[5,4-c]pyridin-2-yl)-3,8-diazabicyclo[3.2.1]octane-3-carboxylate (80 mg, 0.23 mmol, Intermediate 9) was added and the mixture was stirred at room temperature for 1 hour. The mixture was purified with silica column chromatography (0% to 100% ethyl acetate in n-heptane) to afford tert-butyl 8-(5-(2-(2-(difluoromethoxy)phenoxy)acetyl)-4,5,6,7-tetrahydrothiazolo[5,4-c]pyridin-2-yl)-3,8-diazabicyclo[3.2.1]octane-3-carboxylate (158 mg, 99%) as a light yellow solid. LCMS (Method B): t_{R} 2.14 min, 99%, MS (ESI) 551.2 (M−H)⁺.

To a solution of tert-butyl 8-(5-(2-(2-(difluoromethoxy)phenoxy)acetyl)-4,5,6,7-tetrahydrothiazolo[5,4-c]pyridin-2-yl)-3,8-diazabicyclo[3.2.1]octane-3-carboxylate (157 mg, 0.29 mmol) in dichloromethane (1 mL) was added. trifluoroacetic acid (0.44 mL, 5.7 mmol) and the mixture was stirred at room temperature for 30 minutes. The mixture was concentrated and redissolved in 7N ammonia in methanol (1 mL). The solution was purified with preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-6,7-dihydrothiazolo[5,4-c]pyridin-5(4H)-yl)-2-(2-(difluoromethoxy)phenoxy)ethan-1-one (51 mg, 39%, Example 252) as a light yellow solid. ¹H-NMR: δ_{H} (400 MHz, CDCl₃, mixture of rotamers) 7.21-7.13 (2H, m), 7.07-6.94 (2H, m), 6.85-6.41 (1H, m), 4.84 (2H, d, J 13.0), 4.67-4.57 (2H, m), 4.18-4.06 (2H, m), 3.94-3.79 (2H, m), 3.22 (2H, d, J 12.2), 2.82-2.61 (4H, m), 2.12-1.98 (2H, m), 1.98-1.88 (2H, m); UPLC (Method A): t_{R} 1.60 min, 98%, MS (ESI) 451.2 (M+H)⁺.

### Example 253: Synthesis of 2-(2-(2-(3,8-diazabicyclo[3.2.1]octan-8-yl)-6,7-dihydrothiazolo[5,4-c]pyridin-5(4H)-yl)-2-oxoethoxy)-5-fluoro-3-methoxybenzonitrile

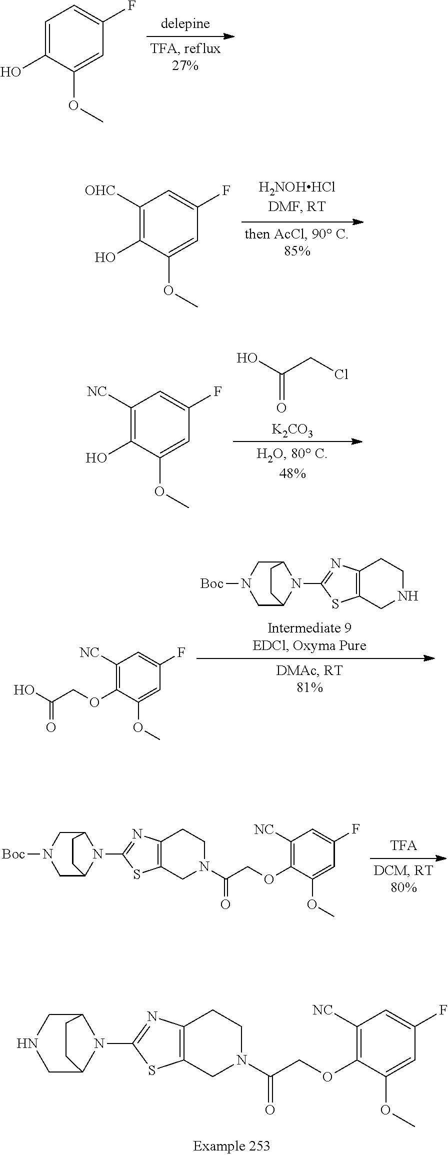

To a solution of 4-fluoro-2-methoxyphenol (553 mg, 3.89 mmol) in trifluoroacetic acid (5 mL) was added hexamethylenetetramine (2.0 g, 14 mmol) and the mixture was refluxed for 4 hours. The mixture was cooled to room temperature, 50% sulfuric acid in water (10 mL) was added and the mixture was stirred for 45 minutes. The mixture was diluted with water and extracted with ethyl acetate twice. The combined organic layers were washed with water, 1 M hydrochloric acid solution and brine. The organic layer was dried with sodium sulfate and concentrated in vacuo. The residue was purified with silica column chromatography (10% ethyl acetate in n-heptane) to afford 5-fluoro-2-hydroxy-3-methoxybenzaldehyde (180 mg, 27%) as a yellow liquid. ¹H-NMR: δ_{H} (400 MHz, CDCl₃) δ 10.82 (1H, s), 9.87 (1H, s), 6.91-6.84 (2H, m), 3.92 (3H, s).; LCMS (Method A): t_{R} 1.55 min, 100%, MS (ESI) 171.0 (M+H)⁺.

A solution of 5-fluoro-2-hydroxy-3-methoxybenzaldehyde (178 mg, 1.05 mmol) and hydroxylamine hydrochloride (72 mg, 1.05 mmol) in N,N-dimethylformamide (5 mL) was stirred at room temperature for 30 minutes. Next, acetyl chloride (0.07 mL, 1.05 mmol) was added dropwise and the mixture was heated to 90° C. for 3 days. The mixture was cooled to room temperature, diluted with ethyl acetate and washed with brine. The organic layer was dried with sodium sulfate and concentrated in vacuo. The residue was purified with silica column chromatography (20% to 50% ethyl acetate in n-heptane) to afford 5-fluoro-2-hydroxy-3-methoxybenzonitrile (149 mg, 85%) as a white solid. ¹H-NMR: δ_{H} (400 MHz, CDCl₃) 6.86-6.77 (2H, m), 6.02 (1H, s), 3.94 (3H, s); LCMS (Method A): t_{R} 1.60 min, 100%, MS (ESI) 168.0 (M+H)⁺.

To a suspension of 5-fluoro-2-hydroxy-3-methoxybenzonitrile (100 mg, 0.60 mmol) and chloroacetic acid (90 mg, 0.96 mmol) in water (1.20 mL) was added potassium carbonate (215 mg, 1.56 mmol). The mixture was heated to 80° C. for 2 days. The mixture was cooled to room temperature, diluted with water and transferred to a separatory funnel. The aqueous layer (pH ˜8) was acidified with 2 m hydrochloric acid solution to pH ˜3 and extracted with ethyl acetate twice. The combined organic layers were washed with brine, dried with sodium sulfate and concentrated in vacuo to afford 2-(2-cyano-4-fluoro-6-methoxyphenoxy)acetic acid (65 mg, 48%) as a light orange solid. ¹H-NMR: δ_{H} (400 MHz, CDCl₃) 6.93-6.85 (2H, m), 4.81 (2H, s), 3.91 (3H, s); LCMS (Method A): t_{R} 1.63 min, 95%, MS (ESI) 224.0 (M−H)⁺.

To a solution of 2-(2-cyano-4-fluoro-6-methoxyphenoxy)acetic acid (48 mg, 0.21 mmol) and tert-butyl 8-(4,5,6,7-tetrahydrothiazolo[5,4-c]pyridin-2-yl)-3,8-diazabicyclo[3.2.1]octane-3-carboxylate (75 mg, 0.21 mmol) in N,N-dimethylformamide (2 mL) was added Oxyma Pure (30 mg, 0.21 mmol) and N-(3-dimethylaminopropyl)-N-ethylcarbodiimide hydrochloride (49 mg, 0.269 mmol). The mixture was stirred at room temperature for 3 days. The mixture was partitioned between water and ethyl acetate and the aqueous layer was extracted once more with ethyl acetate. The combined organic layers were washed with brine, dried over sodium sulfate and concentrated in vacuo. The residue was purified with silica column chromatography (60% to 100% ethyl acetate in n-heptane) to afford (97 mg, 81%) as a colorless gum. LCMS (Method B): t_{R} 2.08 min, 98%, MS (ESI) 558.2 (M+H)⁺.

A solution of tert-butyl 8-(5-(2-(2-cyano-4-fluoro-6-methoxyphenoxy)acetyl)-4,5,6,7-tetrahydrothiazolo[5,4-c]pyridin-2-yl)-3,8-diazabicyclo[3.2.1]octane-3-carboxylate (97 mg, 0.17 mmol) in dichloromethane (2 mL) and trifluoroacetic acid (0.27 mL, 3.5 mmol was stirred at room temperature for 16 hours. The mixture was concentrated in vacuo and coevaporated with dichloromethane twice. The residue was purified with preparative reversed phase chromatography (Waters XSelect™ CSH C18 column, ammonium bicarbonate in water/acetonitrile) and lyophilized to afford 2-(2-(2-(3,8-diazabicyclo[3.2.1]octan-8-yl)-6,7-dihydrothiazolo[5,4-c]pyridin-5(4H)-yl)-2-oxoethoxy)-5-fluoro-3-methoxybenzonitrile (64 mg, 80%, Example 253) as a white solid. ¹H-NMR: δ_{H} (400 MHz, CDCl₃, mixture of rotamers) 6.92-6.80 (2H, m), 4.87 (2H, d, J 13.0), 4.79-4.59 (2H, m), 4.21-4.05 (2H, m), 3.98-3.75 (5H, m), 3.22 (2H, d, J 12.3), 2.90-2.58 (4H, m), 2.13-1.87 (4H, m); UPLC (Method A): t_{R} 2.31 min, 99%, MS (ESI) 458.2 (M+H)⁺.

### Examples 254 and 255: Synthesis of both enantiomers of 1-(2-(3,8-diazabicyclo[3.2.1]octan-8-yl)-6,7-dihydrothiazolo[5,4-c]pyridin-5(4H)-yl)-2-cyclopentyl-2-hydroxypropan-1-one

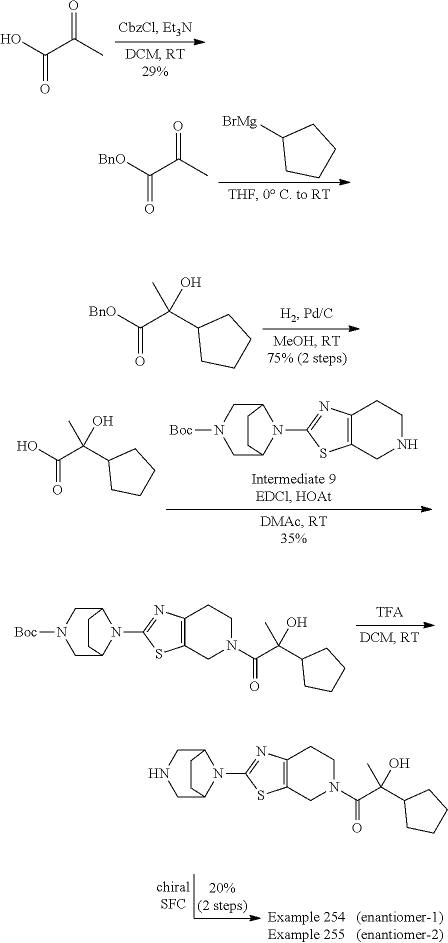

To a solution of pyruvic acid (1.60 mL, 22.7 mmol) and triethylamine (3.17 mL, 22.7 mmol) in dichloromethane (20 mL) was slowly added benzyl chloroformate (3.24 mL, 22.77 mmol). The mixture was stirred at room temperature for 2 hours. The mixture was diluted with diethyl ether and washed with 1M hydrochloric acid solution twice followed by sodium bicarbonate solution once. The organic layer was washed with brine, dried with sodium sulfate and concentrated to afford a dark yellow oil. The oil was purified with silica column chromatography (0% to 100% ethyl acetate in n-heptane) to afford benzyl 2-oxopropanoate (1.2 g, 29%) as a light yellow oil. ¹H-NMR: δ_{H} (400 MHz, CDCl₃) 7.44-7.32 (5H, m), 5.28 (2H, s), 2.48 (3H, s).

Under argon, benzyl 2-oxopropanoate (1.2 g, 6.73 mmol) was dissolved in tetrahydrofuran (50 mL) and cooled to 0° C. Next, 2M cyclopentylmagnesium chloride solution in diethyl ether (3.70 mL, 7.41 mmol) was added and the mixture was allowed to reach room temperature and stirred for 16 hours. The mixture was quenched with saturated ammonium chloride solution and extracted with ethyl acetate twice. The combined organic layers were dried with sodium sulfate and concentrated to afford benzyl 2-cyclopentyl-2-hydroxypropanoate (1.67 g, quant.) as a yellow gum. This was taken forward into the next step without further purification. Under argon atmosphere, benzyl 2-cyclopentyl-2-hydroxypropanoate (1.6 g, 6.44 mmol) was dissolved in methanol (50 mL) and 10% palladium on carbon (0.69 g, 0.64 mmol) was added. Hydrogen atmosphere was introduced and the mixture was stirred at room temperature for 2 hours. The mixture was filtered through Celite® and concentrated to afford 2-cyclopentyl-2-hydroxypropanoic acid (763 mg, 75%) as a yellow oil. This was taken forward into the next step without further purification.

A solution of tert-butyl 8-(4,5,6,7-tetrahydrothiazolo[5,4-c]pyridin-2-yl)-3,8-diazabicyclo[3.2.1]octane-3-carboxylate (100 mg, 0.29 mmol), 2-cyclopentyl-2-hydroxypropanoic acid (54 mg, 0.34 mmol), N-(3-dimethylaminopropyl)-N′-ethylcarbodiimide hydrochloride (109 mg, 0.57 mmol) and 1-hydroxy-7-azabenzotriazole (10 mg, 0.07 mmol) in N,N-dimethylacetamide (1 mL) was stirred at room temperature for 16 hours. The mixture was purified with preparative reversed phase chromatography (Waters XSelect™ CSH C18 column, ammonium bicarbonate in water/acetonitrile) and lyophilized to afford tert-butyl 8-(5-(2-cyclopentyl-2-hydroxypropanoyl)-4,5,6,7-tetrahydrothiazolo[5,4-c]pyridin-2-yl)-3,8-diazabicyclo[3.2.1]octane-3-carboxylate (60 mg, 35%) as a colorless gum. LCMS (Method A): t_{R} 2.14 min, 83%, MS (ESI) 491.2 (M+H)⁺.

To a solution of tert-butyl 8-(5-(2-cyclopentyl-2-hydroxypropanoyl)-4,5,6,7-tetrahydrothiazolo[5,4-c]pyridin-2-yl)-3,8-diazabicyclo[3.2.1]octane-3-carboxylate (60 mg, 0.12 mmol) in dichloromethane (6 mL) was added trifluoroacetic acid (0.50 mL, 6.5 mmol). The mixture was stirred at room temperature for 4 hours. The mixture was purified with SCX (ion exchange) chromatography (washed with methanol and eluted with 3.5 M ammonia in methanol), concentrated in vacuo and lyophilized to afford a white solid. The solid was separated by chiral prep-SFC separation (Phenomenex Lux i-Cellulose-5, CO₂/20 mM NH₃ in MeOH eluent) affording the isolated enantiomers. First eluting isomer (5 mg, 10%, Example 254): ¹H-NMR: δ_{H} (400 MHz, CDCl₃) 4.68 (2H, s), 4.26-4.07 (2H, m), 4.03-3.83 (2H, m), 3.22 (1.5H, d, J 12.1), 2.93-2.62 (4H, m), 2.51 (0.5H, d, J 10.8), 2.33-2.19 (1H, m), 2.13-1.87 (4H, m), 1.77-1.57 (5H, m), 1.56-1.40 (5H, m), 1.39-1.28 (2H, m); UPLC (Method A): t_{R} 1.45 min, 100%, MS (ESI) 391.2 (M+H)⁺; SFC (Phenomenex Lux i-Cellulose-5, CO₂/20 mM NH₃ in MeOH eluent): t_{R} 3.93 min, 100%. Second eluting isomer (5 mg, 10%, Example 255): ¹H-NMR: δ_{H} (400 MHz, CDCl₃) 4.68 (2H, s), 4.26-4.07 (2H, m), 4.03-3.83 (2H, m), 3.22 (1.5H, d, J 12.1), 2.93-2.62 (4H, m), 2.51 (0.5H, d, J 10.8), 2.33-2.19 (1H, m), 2.13-1.87 (4H, m), 1.77-1.57 (5H, m), 1.56-1.40 (5H, m), 1.39-1.28 (2H, m); UPLC (Method A): t_{R} 1.45 min, 100%, MS (ESI) 391.2 (M+H)⁺; SFC (Phenomenex Lux i-Cellulose-5, CO₂/20 mM NH₃ in MeOH eluent): t_{R} 4.20 min, 100%.

The following examples (256-266) were prepared using procedures analogous to Example 98, using the appropriate starting materials.

### Example 256: Synthesis of (2-(3,8-diazabicyclo[3.2.1]octan-8-yl)-7,7-dimethyl-6,7-dihydrothiazolo[5,4-c]pyridin-5(4H)-yl)(cyclopentyl)methanone

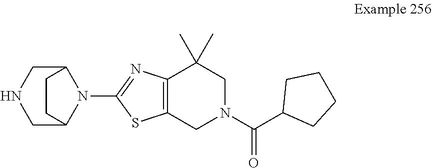 [SMILES: CC1(C)CN(C(=O)C2CCCC2)Cc2sc(N3C4CCC3CNC4)nc21]

Prepared using Intermediate 8 and cyclopentanecarboxylic acid as the acid compound.

¹H-NMR: δ_{H} (400 MHz, CDCl₃, mixture of rotamers) 4.69-4.50 (2H, m), 4.21-4.07 (2H, m), 3.67-3.45 (2H, m), 3.23 (2H, d, J 12.0), 3.07-2.91 (1H, m), 2.75-2.60 (2H, m), 2.14-2.00 (2H, m), 1.98-1.52 (14H, m), 1.35-1.17 (6H, m); UPLC (Method A): t_{R} 1.66 min, 100%, MS (ESI) 375.2 (M+H)⁺.

### Example 257: Synthesis of 1-(2-(3,8-diazabicyclo[3.2.1]octan-8-yl)-7,7-dimethyl-6,7-dihydrothiazolo[5,4-c]pyridin-5(4H)-yl)-3-methylbutan-1-one

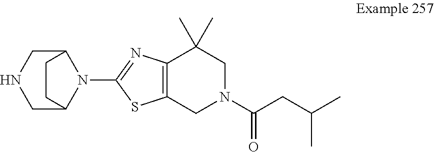 [SMILES: CC(C)CC(=O)N1Cc2sc(N3C4CCC3CNC4)nc2C(C)(C)C1]

Prepared using Intermediate 8 and 3-methylbutanoic acid as the acid compound.

¹H-NMR: δ_{H} (400 MHz, CDCl₃, mixture of rotamers) 4.66-4.46 (2H, m), 4.13 (2H, s), 3.65-3.38 (2H, m), 3.23 (2H, d, J 12.4), 2.64 (2H, d, J 12.4), 2.35-2.11 (3H, m), 2.10-1.99 (2H, m), 1.96-1.86 (2H, m), 1.31-1.19 (7H, m), 1.02-0.95 (6H, m); UPLC (Method A): t_{R} 1.61 min, 96%, MS (ESI) 363.2 (M+H)⁺.

### Example 258: Synthesis of 1-(2-(3,8-diazabicyclo[3.2.1]octan-8-yl)-7,7-dimethyl-6,7-dihydrothiazolo[5,4-c]pyridin-5(4H)-yl)-2-methylpropan-1-one

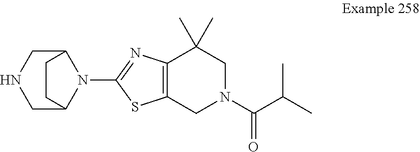 [SMILES: CC(C)C(=O)N1Cc2sc(N3C4CCC3CNC4)nc2C(C)(C)C1]

Prepared using Intermediate 8 and isobutyric acid as the acid compound.

¹H-NMR: δ_{H} (400 MHz, CDCl₃, mixture of rotamers) 4.66-4.49 (2H, m), 4.19-4.06 (2H, m), 3.61 (1H, s), 3.46 (1H, s), 3.23 (2H, d, J 12.3), 3.00-2.80 (1H, m), 2.71-2.59 (2H, m), 2.11-1.96 (2H, m), 1.96-1.84 (2H, m), 1.30-1.11 (12H, m); UPLC (Method A): t_{R} 1.41 min, 96%, MS (ESI) 349.2 (M+H)⁺.

### Example 259: Synthesis of 1-(2-(3,8-diazabicyclo[3.2.1]octan-8-yl)-7,7-dimethyl-6,7-dihydrothiazolo[5,4-c]pyridin-5(4H)-yl)-2-hydroxy-3-methylbutan-1-one

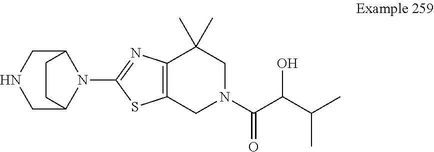 [SMILES: CC(C)C(O)C(=O)N1Cc2sc(N3C4CCC3CNC4)nc2C(C)(C)C1]

Prepared using Intermediate 8 and 2-hydroxy-3-methylbutyric acid as the acid compound.

¹H-NMR: δ_{H} (400 MHz, CDCl₃, mixture of rotamers) 5.08 (0.5H, d, J 16.3), 4.47-4.39 (1H, m), 4.37-4.22 (1.5H, m), 4.13 (2H, s), 3.81-3.17 (5H, m), 2.65 (2H, d, J 11.9), 2.14-2.00 (2H, m), 2.00-1.79 (3H, m), 1.31-1.17 (6H, m), 1.15-1.03 (3H, m), 0.87-0.77 (3H, m); UPLC (Method A): t_{R} 1.42 min, 97%, MS (ESI) 379.2 (M+H)⁺.

### Example 260: Synthesis of 1-(2-(3,8-diazabicyclo[3.2.1]octan-8-yl)-7,7-dimethyl-6,7-dihydrothiazolo[5,4-c]pyridin-5(4H)-yl)-2-(2-oxaspiro[3.5]nonan-7-yl)ethan-1-one

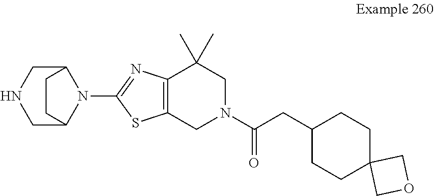 [SMILES: CC1(C)CN(C(=O)CC2CCC3(CC2)COC3)Cc2sc(N3C4CCC3CNC4)nc21]

Prepared using Intermediate 8 and 2-{2-oxaspiro[3.5]nonan-7-yl}acetic acid as the acid compound.

¹H-NMR: δ_{H} (400 MHz, CDCl₃, mixture of rotamers) 4.61 (1.3H, s), 4.48 (0.7H, s), 4.41 (2H, d, J 5.7), 4.33 (2H, d, J 2.9), 4.20-4.04 (2H, m), 3.60 (0.7H, s), 3.40 (1.3H, s), 3.27-3.16 (2H, m), 2.70-2.60 (2H, m), 2.26 (2H, dd, J 6.8, 2.3), 2.19-2.08 (2H, m), 2.08-1.96 (2H, m), 1.96-1.80 (3H, m), 1.79-1.71 (2H, m), 1.53-1.39 (2H, m), 1.22 (6H, d, J 13.6), 1.01-0.85 (2H, m); UPLC (Method A): t_{R} 1.58 min, 98%, MS (ESI) 445.2 (M+H)⁺.

### Example 261: Synthesis of 3-(2-(3,8-diazabicyclo[3.2.1]octan-8-yl)-7,7-dimethyl-6,7-dihydrothiazolo[5,4-c]pyridin-5(4H)-yl)-2-cyclopentyl-3-oxopropanenitrile

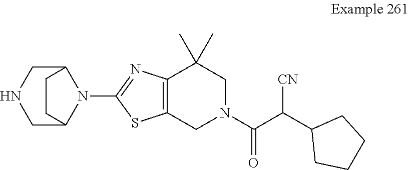 [SMILES: CC1(C)CN(C(=O)C(C#N)C2CCCC2)Cc2sc(N3C4CCC3CNC4)nc21]

Prepared using Intermediate 8 and 2-cyano-2-cyclopentylacetic acid as the acid compound. ¹H-NMR: δ_{H} (400 MHz, CDCl₃, mixture of rotamers) 4.74 (0.6H, d, J 16.4), 4.67-4.50 (1.4H, m), 4.18-4.08 (2H, m), 3.79 (0.4H, d, J 12.9), 3.70-3.58 (1H, m), 3.58-3.37 (1.6H, m), 3.21 (2H, m), 2.71-2.45 (3H, m), 2.13-1.97 (3H, m), 1.97-1.85 (3H, m), 1.78-1.60 (4H, m), 1.59-1.49 (3H, m), 1.34-1.28 (2H, m), 1.28-1.17 (5H, m); UPLC (Method A): t_{R} 1.77 min, 98%, MS (ESI) 414.2 (M+H)⁺.

### Example 262: Synthesis of (2R)-1-(2-(3,8-diazabicyclo[3.2.1]octan-8-yl)-7,7-dimethyl-6,7-dihydrothiazolo[5,4-c]pyridin-5(4H)-yl)-2-cyclopentyl-2-hydroxyethan-1-one

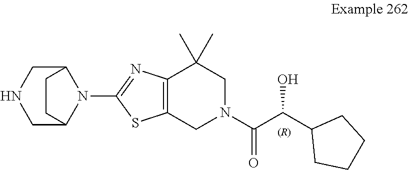

Prepared using Intermediate 8 and (2R)-2-cyclopentyl-2-hydroxyacetic acid as the acid compound.

¹H-NMR: δ_{H} (400 MHz, CDCl₃, mixture of rotamers) 5.18-4.16 (5H, m), 3.83-3.25 (4H, m), 2.93 (2H, d, J 12.4), 2.24-2.04 (5H, m), 1.85-1.58 (4H, m), 1.58-1.34 (4H, m), 1.35-1.12 (6H, m); UPLC (Method A): t_{R} 1.60 min, 99%, MS (ESI) 405.2 (M+H)⁺.

### Example 263: Synthesis of (2-(3,8-diazabicyclo[3.2.1]octan-8-yl)-7,7-dimethyl-6,7-dihydrothiazolo[5,4-c]pyridin-5(4H)-yl)(phenyl)methanone

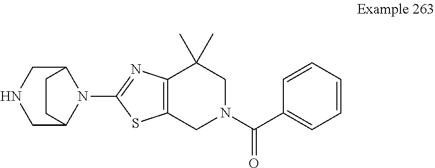 [SMILES: CC1(C)CN(C(=O)c2ccccc2)Cc2sc(N3C4CCC3CNC4)nc21]

Prepared using Intermediate 8 and benzoic acid as the acid compound.

¹H-NMR: δ_{H} (400 MHz, CDCl₃, mixture of rotamers) 7.46-7.36 (5H, m), 4.90-4.34 (2H, m), 4.18-4.06 (2H, m), 3.88-3.32 (2H, m), 3.23 (2H, d, J 12.3), 2.64 (2H, dd, J 12.4, 2.2), 2.11-1.96 (2H, m), 1.96-1.85 (2H, m), 1.41-1.06 (6H, m); UPLC (Method A): t_{R} 1.56 min, 99%, MS (ESI) 383.2 (M+H)⁺.

### Example 264: Synthesis of (2-(3,8-diazabicyclo[3.2.1]octan-8-yl)-7,7-dimethyl-6,7-dihydrothiazolo[5,4-c]pyridin-5(4H)-yl)(6-fluoropyridin-3-yl)methanone

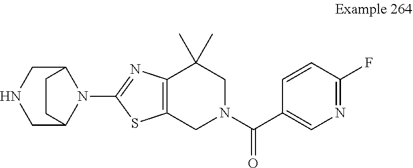 [SMILES: CC1(C)CN(C(=O)c2ccc(F)nc2)Cc2sc(N3C4CCC3CNC4)nc21]

Prepared using Intermediate 8 and 6-fluoronicotinic acid as the acid compound.

¹H-NMR: δ_{H} (400 MHz, CDCl₃, mixture of rotamers) 8.34 (1H, d, J 2.4), 7.96-7.87 (1H, m), 7.02 (1H, dd, J 8.5, 2.8), 4.92-4.36 (2H, m), 4.12 (2H, d, J 4.8), 3.90-3.31 (2H, m), 3.22 (2H, d, J 12.3), 2.65 (2H, dd, J 12.5, 2.3), 2.14-1.96 (2H, m), 1.96-1.84 (2H, m), 1.46-1.06 (6H, m); UPLC (Method A): t_{R} 1.36 min, 96%, MS (ESI) 402.2 (M+H)⁺.

### Example 265: Synthesis of 1-(2′-(3,8-diazabicyclo[3.2.1]octan-8-yl)-4′H-spiro[cyclopropane-1,7′-thiazolo[5,4-c]pyridin]-5′(6′H)-yl)-2-cyclopentylethan-1-one

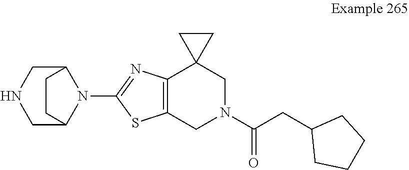 [SMILES: O=C(CC1CCCC1)N1Cc2sc(N3C4CCC3CNC4)nc2C2(CC2)C1]

Prepared using Intermediate 12 and 2-cyclopentylacetic acid as the acid compound.

¹H-NMR: δ_{H} (400 MHz, CDCl₃, mixture of rotamers) 4.78-4.56 (2H, m), 4.17-4.03 (2H, m), 3.77-3.53 (2H, m), 3.30-3.13 (2H, m), 2.63 (2H, dt, J 12.4, 3.2), 2.41 (2H, dd, J 16.5, 7.1), 2.35-2.20 (1H, m), 2.09-1.80 (6H, m), 1.63-1.46 (4H, m), 1.27-1.07 (4H, m), 0.91-0.72 (2H, m); UPLC (Method A): t_{R} 1.83 min, 100%, MS (ESI) 387.2 (M+H)⁺.

### Example 266: Synthesis of 1-(2′-(3,8-diazabicyclo[3.2.1]octan-8-yl)-2,3,5,6-tetrahydro-4′H-spiro[pyran-4,7′-thiazolo[5,4-c]pyridin]-5′(6′H)-yl)-2-cyclopentylethan-1-one

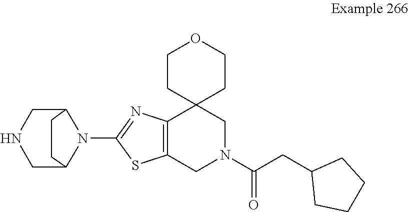 [SMILES: O=C(CC1CCCC1)N1Cc2sc(N3C4CCC3CNC4)nc2C2(CCOCC2)C1]

Prepared using Intermediate 13 and 2-cyclopentylacetic acid as the acid compound.

¹H-NMR: δ_{H} (400 MHz, CDCl₃, mixture of rotamers) 400 MHz, CDCl₃) 4.61 (0.9H, s), 4.52 (1.1H, s), 4.22-4.06 (2.9H, m), 4.00-3.90 (1.1H, m), 3.87 (1.1H, s), 3.81-3.64 (2H, m), 3.59 (0.9H, s), 3.25 (2H, d, J 12.4), 2.72 (2H, d, J 12.4), 2.48-2.39 (2H, m), 2.39-2.24 (1H, m), 2.24-2.13 (1H, m), 2.13-2.01 (3H, m), 2.01-1.93 (2H, m), 1.93-1.81 (2H, m), 1.70-1.47 (5H, m), 1.39 (1H, d, J 13.8), 1.23-1.10 (2H, m); UPLC (Method A): t_{R} 0.85 min, 99%, MS (ESI) 431.5 (M+H)⁺.

### Example 267: Synthesis of 1-(2-(3,8-diazabicyclo[3.2.1]octan-8-yl)-6,6-dimethyl-6,7-dihydrothiazolo[5,4-c]pyridin-5(4H)-yl)-2-cyclopentylethan-1-one

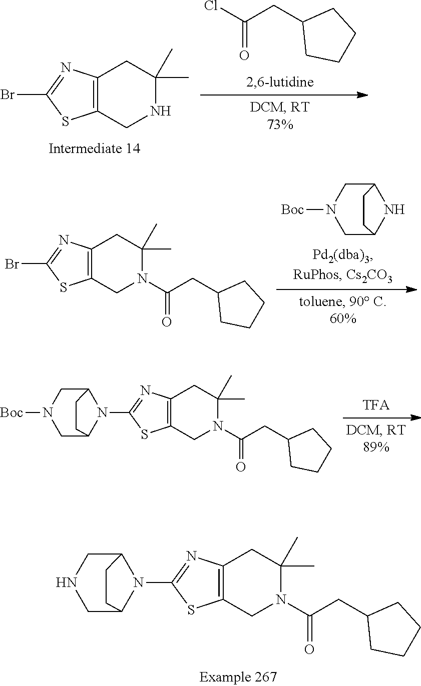 [SMILES: CC1(C)Cc2nc(Br)sc2CN1 ; O=C(Cl)CC1CCCC1 ; CC1(C)Cc2nc(Br)sc2CN1C(=O)CC1CCCC1 ; CC(C)(C)OC(=O)N1CC2CCC(C1)N2 ; CC(C)(C)OC(=O)N1CC2CCC(C1)N2c1nc2c(s1)CN(C(=O)CC1CCCC1)C(C)(C)C2 ; CC1(C)Cc2nc(N3C4CCC3CNC4)sc2CN1C(=O)CC1CCCC1]

To a solution of 2-bromo-6,6-dimethyl-4,5,6,7-tetrahydrothiazolo[5,4-c]pyridine (57 mg, 0.23 mmol, Intermediate 14) in dichloromethane (3 mL) was added cyclopentylacetyl chloride (0.05 mL, 0.35 mmol) and 2,6-lutidine (0.05 mL, 0.46 mmol). The mixture was stirred at room temperature for 16 hours. The mixture was concentrated in vacuo, coated onto silica and purified with silica column chromatography (0% to 100% ethyl acetate in n-heptane) to afford 1-(2-bromo-6,6-dimethyl-6,7-dihydrothiazolo[5,4-c]pyridin-5(4H)-yl)-2-cyclopentylethan-1-one (64 mg, 73%) as a colorless gum. LCMS (Method B): t_{R} 2.18 min, 95%, MS (ESI) 357.1 (M+H)⁺.

Under argon, 1-(2-bromo-6,6-dimethyl-6,7-dihydrothiazolo[5,4-c]pyridin-5(4H)-yl)-2-cyclopentylethan-1-one (64 mg, 0.18 mmol), 3-boc-3,8-diazabicyclo[3.2.1]octane (49 mg, 0.23 mmol) and cesium carbonate (146 mg, 0.45 mmol) were suspended in toluene (0.5 mL). Next, RuPhos (13 mg, 0.03 mmol) and Pd₂(dba)₃ (11 mg, 0.01 mmol) were added and the mixture was stirred at 90° C. for 16 hours. The mixture was filtered over Celite®, washed with acetonitrile and concentrated in vacuo. The residue was dissolved in dichloromethane and purified with silica column chromatography (0% to 100% ethyl acetate in n-heptane) to afford tert-butyl 8-(5-(2-cyclopentylacetyl)-6,6-dimethyl-4,5,6,7-tetrahydrothiazolo[5,4-c]pyridin-5(4H)-yl)-3,8-diazabicyclo[3.2.1]octane-3-carboxylate (56 mg, 60%) as a yellow waxy solid. LCMS (Method B): t_{R} 2.33 min, 95%, MS (ESI) 489.3 (M+H)⁺.

To a solution of tert-butyl 8-(5-(2-cyclopentylacetyl)-6,6-dimethyl-4,5,6,7-tetrahydrothiazolo[5,4-c]pyridin-2-yl)-3,8-diazabicyclo[3.2.1]octane-3-carboxylate (56 mg, 0.12 mmol) in dichloromethane (5 mL) was added trifluoroacetic acid (0.18 mL, 2.29 mmol) and the mixture was stirred at room temperature for 1 hour. The mixture was concentrated in vacuo, redissolved in methanol (3 mL), purified with SCX (ion exchange) chromatography (washed with methanol and eluted with 3.5 M ammonia in methanol) and lyophilized to afford 1-(2-(3,8-diazabicyclo[3.2.1]octan-8-yl)-6,6-dimethyl-6,7-dihydrothiazolo[5,4-c]pyridin-5(4H)-yl)-2-cyclopentylethan-1-one (41 mg, 89%, Example 267) as a yellow solid. ¹H-NMR: δ_{H} (400 MHz, CDCl₃, mixture of rotamers) 4.48-4.37 (2H, m), 4.20-4.07 (2H, m), 3.29-3.16 (2H, m), 2.75-2.61 (4H, m), 2.37 (2H, d, J 7.1), 2.31-2.17 (1H, m), 2.14-2.02 (2H, m), 1.98-1.90 (2H, m), 1.90-1.78 (2H, m), 1.65-1.47 (10H, m), 1.23-1.04 (2H, m); UPLC (Method A): t_{R} 1.79 min, 97%, MS (ESI) 389.4 (M+H)⁺.

The following examples (268-269) were prepared using procedures analogous to Example 267, using the appropriate starting materials.

### Example 268: Synthesis of 1-(2-(3,8-diazabicyclo[3.2.1]octan-8-yl)-6,6-dimethyl-6,7-dihydrothiazolo[5,4-c]pyridin-5(4H)-yl)-2-methylpropan-1-one

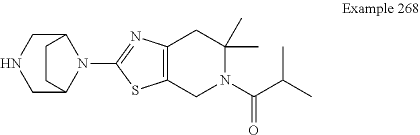 [SMILES: CC(C)C(=O)N1Cc2sc(N3C4CCC3CNC4)nc2CC1(C)C]

Prepared using isobutyryl chloride.

¹H-NMR: δ_{H} (400 MHz, CDCl₃, mixture of rotamers) 4.52-4.43 (2H, m), 4.17-4.10 (2H, m), 3.29-3.19 (2H, m), 2.85-2.75 (1H, m), 2.72 (2H, t, J 1.6), 2.66 (2H, dd, J 12.1, 2.2), 2.13-1.88 (4H, m), 1.54 (6H, s), 1.13-1.08 (6H, m); UPLC (Method A): t_{R} 1.43 min, 98%, MS (ESI) 349.2 (M+H)⁺.

### Example 269: Synthesis of (2-(3,8-diazabicyclo[3.2.1]octan-8-yl)-6,6-dimethyl-6,7-dihydrothiazolo[5,4-c]pyridin-5(4H)-yl)(phenyl)methanone

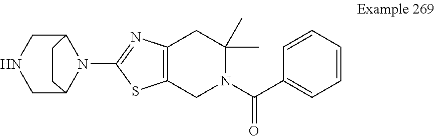 [SMILES: CC1(C)Cc2nc(N3C4CCC3CNC4)sc2CN1C(=O)c1ccccc1]

Prepared using benzoyl chloride.

¹H-NMR: δ_{H} (400 MHz, CDCl₃, mixture of rotamers) 7.55-7.49 (2H, m), 7.46-7.36 (3H, m), 4.39 (2H, t, J 1.8), 4.16-4.05 (2H, m), 3.21 (2H, dd, J 12.4, 1.9), 2.86 (2H, t, J 1.8), 2.65 (2H, dd, J 12.4, 2.5), 2.09-1.88 (4H, m), 1.62 (6H, s); UPLC (Method A): t_{R} 1.61 min, 98%, MS (ESI) 383.2 (M+H)⁺.

### Example 270: Synthesis of 1-(2-(3,8-diazabicyclo[3.2.1]octan-8-yl)-7,7-difluoro-6,7-dihydrothiazolo[5,4-c]pyridin-5(4H)-yl)-2-cyclopentylethan-1-one

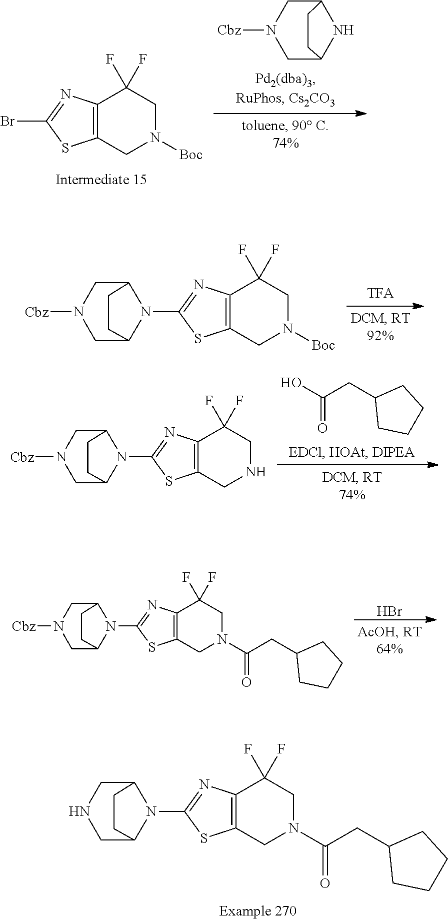

Under argon atmosphere, tert-butyl 2-bromo-7,7-difluoro-6,7-dihydrothiazolo[5,4-c]pyridine-5(4H)-carboxylate (234 mg, 0.66 mmol, Intermediate 15), benzyl 3,8-diazabicyclo[3.2.1]octane-3-carboxylate (211 mg, 0.86 mmol), cesium carbonate (537 mg, 1.65 mmol), RuPhos (49 mg, 0.11 mmol) and Pd₂(dba)₃ (42 mg, 0.05 mmol) were suspended in toluene (2 mL) and the mixture was heated to 90° C. for 16 hours. The mixture was filtered over Celite, washed with acetonitrile and concentrated in vacuo. The residue was dissolved in dichloromethane and purified with silica column chromatography (0% to 100% ethyl acetate in n-heptane) to afford tert-butyl 2-(3-((benzyloxy)carbonyl)-3,8-diazabicyclo[3.2.1]octan-8-yl)-7,7-difluoro-6,7-dihydrothiazolo[5,4-c]pyridine-5(4H)-carboxylate (276 mg, 74%) as a tan solid. ¹H-NMR: δ_{H} (400 MHz, CDCl₃, mixture of rotamers) 7.40-7.29 (5H, m), 5.14 (2H, s), 4.56 (2H, s), 4.28 (2H, s), 4.09-3.96 (2H, m), 3.92 (1H, d, J 13.1), 3.82 (1H, d, J 12.8), 3.39 (1H, d, J 13.0), 3.31 (1H, d, J 12.9), 2.11-1.96 (2H, m), 1.90-1.73 (2H, m), 1.49 (9H, s); LCMS (Method B): t_{R} 2.26 min, 95%, MS (ESI) 521.2 (M+H)⁺.

To a solution of tert-butyl 2-(3-((benzyloxy)carbonyl)-3,8-diazabicyclo[3.2.1]octan-8-yl)-7,7-difluoro-6,7-dihydrothiazolo[5,4-c]pyridine-5(4H)-carboxylate (275 mg, 0.53 mmol) in dichloromethane (2 mL) was added trifluoroacetic acid (0.81 mL, 10.56 mmol) and the mixture was stirred at room temperature for 1 hour. The mixture was concentrated in vacuo, partitioned between saturated aqueous potassium carbonate solution and dichloromethane and the layers were separated. The aqueous layer was extracted with dichloromethane three times and the combined organic layers were filtered, concentrated in vacuo and coevaporated with heptane to afford benzyl 8-(7,7-difluoro-4,5,6,7-tetrahydrothiazolo[5,4-c]pyridin-2-yl)-3,8-diazabicyclo[3.2.1]octane-3-carboxylate (214 mg, 92%) as a purple solid. LCMS (Method B): t_{R} 1.95 min, 96%, MS (ESI) 421.2 (M+H)⁺.

A solution of cyclopentylacetic acid (0.09 mL, 0.71 mmol), N-(3-dimethylaminopropyl)-N′-ethylcarbodiimide hydrochloride (0.13 mL, 0.71 mmol) and 1-hydroxy-7-azabenzotriazole (6.93 mg, 0.05 mmol) in dichloromethane (3 mL) was stirred at room temperature for 5 minutes. benzyl 8-(7,7-difluoro-4,5,6,7-tetrahydrothiazolo[5,4-c]pyridin-2-yl)-3,8-diazabicyclo[3.2.1]octane-3-carboxylate (214 mg, 0.51 mmol) and diisopropylethylamine (0.13 mL, 0.76 mmol) were added and the mixture was stirred at room temperature for 16 hours. The mixture was purified with silica column chromatography (0% to 100% ethyl acetate in n-heptane) to afford benzyl 8-(5-(2-cyclopentylacetyl)-7,7-difluoro-4,5,6,7-tetrahydrothiazolo[5,4-c]pyridin-2-yl)-3,8-diazabicyclo[3.2.1]octane-3-carboxylate (208 mg, 74%) as a white solid. ¹H-NMR: δ_{H} (400 MHz, CDCl₃, mixture of rotamers) 7.41-7.28 (5H, m), 5.14 (2H, s), 4.80-4.66 (1.6H, m), 4.65-4.53 (0.4H, m), 4.36-4.16 (2.4H, m), 4.07-3.97 (1.6H, m), 3.93 (1H, d, J 13.0), 3.82 (1H, d, J 13.0), 3.39 (1H, d, J 12.9), 3.30 (1H, d, J 13.0), 2.52-2.39 (2H, m), 2.37-2.20 (1H, m), 2.12-1.97 (2H, m), 1.96-1.71 (4H, m), 1.69-1.57 (3H, m), 1.56-1.50 (1H, m), 1.23-1.08 (2H, m); LCMS (Method B): t_{R} 2.23 min, 97%, MS (ESI) 531.2 (M+H)⁺.

To a solution of benzyl 8-(5-(2-cyclopentylacetyl)-7,7-difluoro-4,5,6,7-tetrahydrothiazolo[5,4-c]pyridin-2-yl)-3,8-diazabicyclo[3.2.1]octane-3-carboxylate (207 mg, 0.39 mmol) in acetic acid (2 mL) was added 33% hydrobromic acid in acetic acid (0.34 mL, 2.0 mmol) and the mixture was stirred at room temperature for 1 hour. The mixture was poured into diethyl ether (30 mL) and the resulting precipitate was filtered off and washed with diethyl ether. The solids were dissolved in 7N ammonia in methanol (0.5 mL), purified by preparative reversed phase chromatography (Waters XSelect™ CSH C18 column, ammonium bicarbonate in water/acetonitrile) and lyophilized to afford 1-(2-(3,8-diazabicyclo[3.2.1]octan-8-yl)-7,7-difluoro-6,7-dihydrothiazolo[5,4-c]pyridin-5(4H)-yl)-2-cyclopentylethan-1-one (99 mg, 63% yield, Example 270) as a white solid. ¹H-NMR: δ_{H} (400 MHz, CDCl₃, mixture of rotamers) 4.77-4.65 (1.6H, m), 4.59 (0.4H, s), 4.28-4.13 (2.4H, m), 4.08-3.96 (1.6H, m), 3.20 (2H, dd, J 12.5, 1.7), 2.68 (2H, dd, J 12.5, 2.3), 2.53-2.38 (2H, m), 2.38-2.22 (1H, m), 2.15-1.99 (2H, m), 1.99-1.92 (2H, m), 1.92-1.80 (2H, m), 1.69-1.62 (2H, m), 1.60-1.50 (2H, m), 1.25-1.07 (2H, m); UPLC (Method A): 1.66 min, 99%, MS (ESI) 397.2 (M+H)⁺.

### Example 271: Synthesis of (S)-2-cyclopentyl-1-(2-(hexahydropyrrolo[1,2-a]pyrazin-2(1H)-yl)-6,7-dihydrothiazolo[5,4-c]pyridin-5(4H)-yl)ethan-1-one

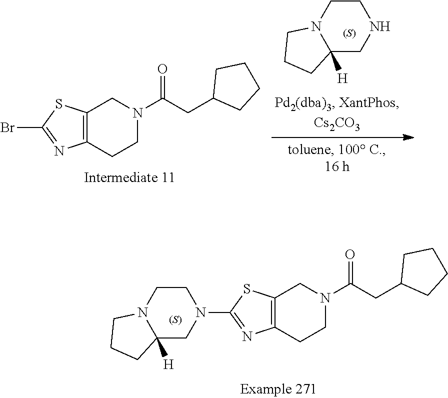

To a stirred solution of 1-(2-bromo-6,7-dihydrothiazolo[5,4-c]pyridin-5(4H)-yl)-2-cyclopentylethan-1-one (Intermediate 11, 350 mg, 1.06 mmol) in 1,4-dioxane (10 mL) were added(S)-octahydropyrrolo[1,2-a]pyrazine (161 mg, 1.28 mmol) and cesium carbonate (691 mg, 2.12 mmol) at room temperature and the resulting mixture was degassed by bubbling nitrogen for 10 minutes. Subsequently, tris(dibenzylideneacetone)dipalladium(0) (97.0 mg, 0.106 mmol) and XantPhos (123 mg, 0.213 mmol) were added and the mixture was heated at 100° C. for 16 h. After completion, the reaction mixture was filtered through a pad of Celite®, washing with DCM. The filtrate was concentrated to obtain a crude residue that was diluted with DCM and washed with water and brine. The organic phase was dried (Na₂SO₄) and evaporated to a residue that was processed by reversed-phase preparative HPLC using ammonium bicarbonate as a buffer. Pure fractions were lyophilized to afford (S)-2-cyclopentyl-1-(2-(hexahydropyrrolo[1,2-a]pyrazin-2(1H)-yl)-6,7-dihydrothiazolo[5,4-c]pyridin-5(4H)-yl)ethan-1-one (15 mg, 4%, Example 271) as an off-white solid. ¹H-NMR: δ_{H} (400 MHz, DMSO-d₆, 80° C.) 4.51 (2H, s), 3.93-3.89 (1H, m), 3.76-3.70 (3H, m), 3.06-3.00 (3H, m), 2.72 (1H, dd, J 11.7 and 10.5), 2.63-2.50 (2H, m), 2.43-2.37 (2H, m), 2.26-1.98 (4H, m), 1.88-1.65 (5H, m), 1.65-1.45 (4H, m), 1.41-1.31 (1H, m), 1.23-1.10 (2H, m); MS (ESI) 375.3 (M+H)⁺.

### Example 272: Synthesis of (R)-2-cyclopentyl-1-(2-(hexahydropyrrolo[1,2-a]pyrazin-2(1H)-yl)-6,7-dihydrothiazolo[5,4-c]pyridin-5(4H)-yl)ethan-1-one

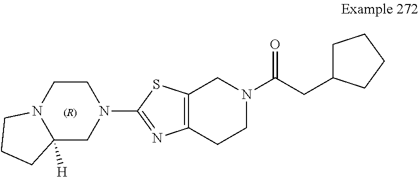

Prepared according to the method of Example 271, using (R)-octahydropyrrolo[1,2-a]pyrazine instead of (S)-octahydropyrrolo[1,2-a]pyrazine.

¹H-NMR: δ_{H} (400 MHz, DMSO-d₆, 80° C.) 4.51 (2H, s), 3.93-3.89 (1H, m), 3.76-3.70 (3H, m), 3.06-3.00 (3H, m), 2.72 (1H, dd, J 11.7 and 10.5), 2.63-2.50 (2H, m), 2.43-2.37 (2H, m), 2.26-1.98 (4H, m), 1.88-1.65 (5H, m), 1.65-1.45 (4H, m), 1.41-1.31 (1H, m), 1.23-1.10 (2H, m); MS (ESI) 375.2 (M+H)⁺.

### Example 273: Synthesis of 1-(2-(3,9-diazabicyclo[3.3.1]nonan-9-yl)-6,7-dihydrothiazolo[5,4-c]pyridin-5(4H)-yl)-2-cyclopentylethan-1-one, TFA Salt

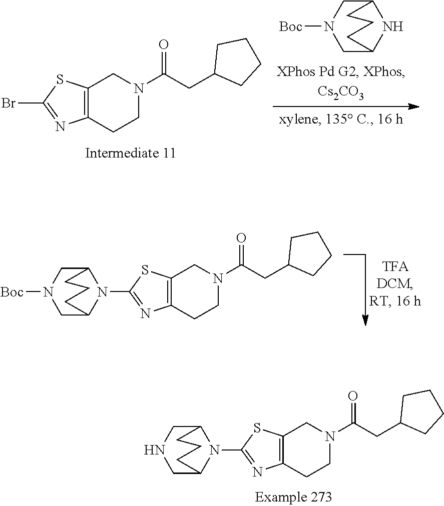 [SMILES: O=C(CC1CCCC1)N1CCc2nc(Br)sc2C1 ; CC(C)(C)OC(=O)N1CC2CCCC(C1)N2 ; CC(C)(C)OC(=O)N1CC2CCCC(C1)N2c1nc2c(s1)CN(C(=O)CC1CCCC1)CC2 ; O=C(CC1CCCC1)N1CCc2nc(N3C4CCCC3CNC4)sc2C1]

To a stirred solution of 1-(2-bromo-6,7-dihydrothiazolo[5,4-c]pyridin-5(4H)-yl)-2-cyclopentylethan-1-one (Intermediate 11, 175 mg, 0.532 mmol) in xylene (2 mL) were added tert-butyl 3,9-diazabicyclo[3.3.1]nonane-3-carboxylate (80.0 mg, 0.353 mmol), cesium carbonate (346 mg, 1.06 mmol), XPhos (33.7 mg, 0.071 mmol) and XPhos Pd G2 (27.8 mg, 0.035 mmol) under continuous bubbling of nitrogen. The resulting reaction mixture was heated at 135° C. for 16 h. After completion, the reaction mixture was evaporated and the crude material processed by flash column chromatography (0 to 50% EtOAc/petroleum eluent), affording tert-butyl 9-(5-(2-cyclopentylacetyl)-4,5,6,7-tetrahydrothiazolo[5,4-c]pyridin-2-yl)-3,9-diazabicyclo[3.3.1]nonane-3-carboxylate (50 mg, 30%) as a pale yellow oil.

To a stirred solution of tert-butyl 9-(5-(2-cyclopentylacetyl)-4,5,6,7-tetrahydrothiazolo[5,4-c]pyridin-2-yl)-3,9-diazabicyclo[3.3.1]nonane-3-carboxylate (100 mg, 0.211 mmol) in DCM (5 mL) was added TFA (240 mg, 2.11 mmol) under nitrogen atmosphere. The resulting mixture was stirred at room temperature for 16 h. After completion, the reaction mixture was concentrated, diluted with water (5 mL) and basified with saturated aq. sodium bicarbonate solution (5 mL). The organic components were extracted with 10% MeOH in DCM (10 mL×2). The combined organic layers were washed with brine (10 mL), dried (Na₂SO₄) and evaporated. The crude product (60 mg) was purified by reversed-phase preparative HPLC purification using TFA as a buffer. Pure fractions were lyophilized to obtain 1-(2-(3,9-diazabicyclo[3.3.1]nonan-9-yl)-6,7-dihydrothiazolo[5,4-c]pyridin-5(4H)-yl)-2-cyclopentylethan-1-one, TFA salt (10 mg, 9%, Example 273) as a pale yellow solid. ¹H-NMR: δ_{H} (400 MHz, DMSO-d₆, mixture of rotamers) 9.44-9.31 (1H, m), 8.04-7.90 (1H, m), 4.61-4.45 (2H, m), 4.17 (2H, br s), 3.73-3.38 (2H, m), 3.45-3.37 (2H, m), 3.31-3.20 (2H, m), 2.65-2.57 (2H, m), 2.46-2.36 (2H, m), 2.22-2.03 (2H, m), 2.00-1.85 (2H, m), 1.81-1.68 (4H, m), 1.67-1.41 (5H, m), 1.19-1.05 (2H, m); MS (ESI) 375.4 (M+H)⁺.

### Example 274: Synthesis of 3,3-dimethyl-1-(2-(piperazin-1-yl)-6,7-dihydrothiazolo[5,4-c]pyridin-5(4H)-yl) butan-1-one

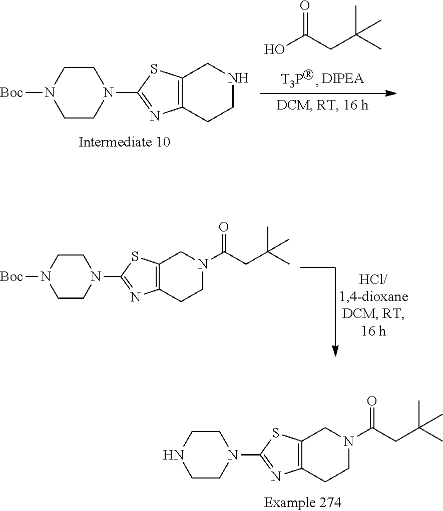

To a stirred solution of tert-butyl 4-(4,5,6,7-tetrahydrothiazolo[5,4-c]pyridin-2-yl)piperazine-1-carboxylate (Intermediate 10, 400 mg, 1.23 mmol) in THF (10 mL) were added 3,3-dimethylbutanoic acid (215 mg, 1.85 mmol), n-propylphosphonic acid anhydride, cyclic trimer (50% in EtOAc; 1.47 mL, 2.47 mmol) and DIPEA (1.07 mL, 6.16 mmol) under nitrogen atmosphere and the resulting mixture was heated at 50° C. for 16 h. After completion, the reaction mixture was diluted with DCM (100 mL) and washed with water (2×50 mL) and brine (50 mL). The combined organic layers were dried (Na₂SO₄) and evaporated. The crude material thus obtained was triturated with 20% EtOAc/petroleum to give a solid that was collected by filtration and dried, affording tert-butyl 4-(5-(3,3-dimethylbutanoyl)-4,5,6,7-tetrahydrothiazolo[5,4-c]pyridin-2-yl)piperazine-1-carboxylate (420 mg, 81%) as an off-white solid.

To a stirred solution of tert-butyl 4-(5-(3,3-dimethylbutanoyl)-4,5,6,7-tetrahydrothiazolo[5,4-c]pyridin-2-yl)piperazine-1-carboxylate (420 mg, 0.994 mmol) in DCM (10 mL) at room temperature was added 4M HCl in dioxane (0.994 mL, 3.98 mmol). After 16 h the reaction mixture was evaporated. The residue obtained was dissolved in water, basified using aq. NaHCO₃ solution and then extracted with 10% methanol in DCM (2×50 mL). The combined organic layers were washed with water (25 mL) and brine (25 mL), dried (Na₂SO₄) and evaporated. The crude material was triturated with 20% EtOAc/petroleum to give solid that was processed by lyophilization with acetonitrile and water, affording 3,3-dimethyl-1-(2-(piperazin-1-yl)-6,7-dihydrothiazolo[5,4-c]pyridin-5(4H)-yl) butan-1-one (280 mg, 87%, Example 274) as an off-white solid. ¹H-NMR: δ_{H} (400 MHz, DMSO-d₆, mixture of rotamers) 4.59-4.56 (2H, 2×s, rotamers), 3.74 (2H, J5.8, t), 3.32-3.23 (4H, m), 2.77-2.75 (4H, m), 2.59-2.56 (2H, m), 2.32-2.28 (2H, 2×s, rotamers), 1.09-0.99 (9H, 2×s, rotamers); MS (ESI) 323.2 (M+H)⁺.

### Example 275: Synthesis of (R)-2-cyclopentyl-1-(2-(2-(hydroxymethyl)piperazin-1-yl)-6,7-dihydrothiazolo[5,4-c]pyridin-5(4H)-yl)ethan-1-one TFA Salt

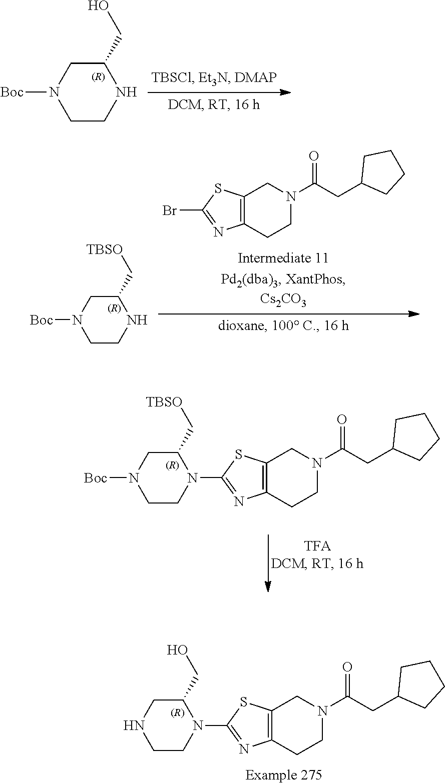

To a stirred solution of tert-butyl (R)-3-(hydroxymethyl)piperazine-1-carboxylate (300 mg, 1.39 mmol) in DCM (20 mL) at 0° C. were added TBDMSCl (1.05 g, 6.93 mmol), triethylamine (0.387 mL, 2.78 mmol) and DMAP (17.0 mg, 0.139 mmol). The reaction mixture was then brought to room temperature. After 16 h the mixture was diluted with saturated aq. NaHCO₃ solution (20 mL) and extracted with 10% MeOH in DCM (30 mL×2). The combined organic extracts were washed with brine (30 mL), dried (Na₂SO₄) and evaporated to afford tert-butyl (R)-3-(((tert-butyldimethylsilyl)oxy)methyl)piperazine-1-carboxylate (400 mg, 87%) as a gum.

To a stirred solution of 1-(2-bromo-6,7-dihydrothiazolo[5,4-c]pyridin-5(4H)-yl)-2-cyclopentylethan-1-one (Intermediate 11, 200 mg, 0.607 mmol) and tert-butyl (R)-3-(hydroxymethyl)piperazine-1-carboxylate (402 mg, 1.22 mmol), in 1,4-dioxane (5 mL) were added cesium carbonate (594 mg, 1.82 mmol), XantPhos (70.3 mg, 0.121 mmol) and Pd₂(dba)₃ (55.6 mg, 0.061 mmol) under continuous purging of nitrogen. Subsequently, and Pd₂(dba)₃ (55.6 mg, 0.061 mmol) was added, and the resulting mixture was heated at 100° C. After 16 h the reaction mixture was diluted with ice-cold water (50 mL) and extracted with EtOAc (50 mL×3). The combined organic layers were washed with brine (30 mL), dried (Na₂SO₄) and evaporated. The resulting residue was processed by flash column chromatography using (50% EtOAc/petroleum eluent) to afford tert-butyl (R)-3-(((tert-butyldimethylsilyl)oxy)methyl)-4-(5-(2-cyclopentylacetyl)-4,5,6,7-tetrahydrothiazolo[5,4-c]pyridin-2-yl)piperazine-1-carboxylate (200 mg, 57%) as a yellow oil.

To a stirred solution of tert-butyl (R)-3-(((tert-butyldimethylsilyl)oxy)methyl)-4-(5-(2-cyclopentylacetyl)-4,5,6,7-tetrahydrothiazolo[5,4-c]pyridin-2-yl)piperazine-1-carboxylate (150 mg, 0.259 mmol) in DCM (5 mL) at room temperature was added TFA (0.991 mL, 11.4 mmol). After 16 h the reaction mixture was evaporated to give a residue that was processed by reversed-phase preparative HPLC using TFA as a buffer. Pure fractions were lyophilized to afford (R)-2-cyclopentyl-1-(2-(2-(hydroxymethyl)piperazin-1-yl)-6,7-dihydrothiazolo[5,4-c]pyridin-5(4H)-yl)ethan-1-one TFA salt (10 mg, 8%, Example 275) as a gummy solid. ¹H-NMR: δ_{H} (400 MHz, DMSO-d₆, mixture of rotamers) 9.11 (1H, br d, J 10.8), 8.35 (1H, br q, J 10.5), 4.55-4.50 (2H, m), 4.10-4.03 (1H, m), 3.89-3.81 (1H, m), 3.80-3.74 (1H, m), 3.74-3.67 (3H, m), 3.50-3.39 (2H, m), 3.33-3.26 (1H, m), 3.26-3.15 (1H, m), 3.14-3.00 (1H, m), 2.62-2.53 (2H, m), 2.44-2.37 (2H, m), 2.22-2.08 (1H, m), 1.81-1.65 (2H, m), 1.65-1.41 (4H, m), 1.18-1.04 (2H, m); MS (ESI) 365.2 (M+H)⁺.

The following examples (276-279) were prepared using procedures analogous to Example 273, using the appropriate amine in place of tert-butyl 3,9-diazabicyclo[3.3.1]nonane-3-carboxylate.

### Example 276: Synthesis of 1-(2-(3,8-diazabicyclo[3.2.1]octan-3-yl)-6,7-dihydrothiazolo[5,4-c]pyridin-5(4H)-yl)-2-cyclopentylethan-1-one

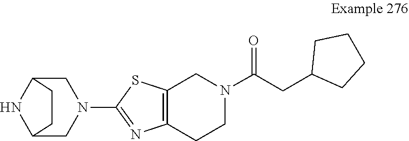 [SMILES: O=C(CC1CCCC1)N1CCc2nc(N3CC4CCC(C3)N4)sc2C1]

Prepared using tert-butyl 3,8-diazabicyclo[3.2.1]octane-8-carboxylate.

¹H-NMR: δ_{H} (400 MHz, DMSO-d₆, mixture of rotamers) 4.52-4.45 (2H, m), 3.74-3.65 (2H, m), 3.49-3.43 (2H, m), 3.41-3.34 (2H, m), 3.05-2.99 (2H, m), 2.61-2.52 (2H, m), 2.44-2.35 (2H, m), 2.21-2.07 (1H, m), 1.80-1.42 (10H, m), 1.18-1.05 (2H, m); MS (ESI) 361.2 (M+H)⁺.

### Example 277: Synthesis of 1-(2-((1S,4S)-2,5-diazabicyclo[2.2.1]heptan-2-yl)-6,7-dihydrothiazolo[5,4-c]pyridin-5(4H)-yl)-2-cyclopentylethan-1-one

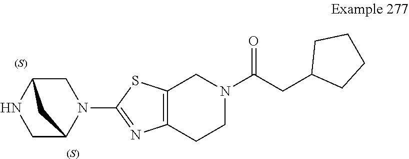

Prepared using tert-butyl (1S,4S)-2,5-diazabicyclo[2.2.1]heptane-2-carboxylate.

¹H-NMR: δ_{H} (400 MHz, DMSO-d₆, mixture of rotamers) 4.54-4.42 (2H, m), 4.30-4.24 (1H, m), 3.76-3.66 (2H, m), 3.66-3.60 (1H, m), 3.45-3.40 (1H, m), 3.11-3.06 (1H, m), 2.89-2.84 (2H, m), 2.61-2.55 (2H, m), 2.44-2.36 (2H, m), 2.21-2.08 (1H, m), 1.81-1.68 (3H, m), 1.67-1.41 (5H, m), 1.18-1.05 (2H, m); MS (ESI) 347.2 (M+H)⁺.

### Example 278: Synthesis of 1-(2-(2,5-diazabicyclo[2.2.2]octan-2-yl)-6,7-dihydrothiazolo[5,4-c]pyridin-5(4H)-yl)-2-cyclopentylethan-1-one

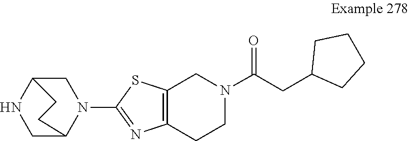 [SMILES: O=C(CC1CCCC1)N1CCc2nc(N3CC4CCC3CN4)sc2C1]

Prepared using tert-butyl 2,5-diazabicyclo[2.2.2]octane-2-carboxylate.

¹H-NMR: δ_{H} (400 MHz, CD₃OD, mixture of rotamers) 4.62-4.57 (2H, 2×s, rotamers), 4.00-3.96 (1H, m), 3.90-3.81 (2H, 2×t, J 5.8, rotamers), 3.67 (1H, dt, J 10.5 and 2.6), 3.51 (1H, dd, J 10.6 and 1.7), 3.31-3.25 (1H, m), 3.22-3.16 (1H, m), 3.11 (1H, dd, J 11.2 and 1.7), 2.73-2.60 (2H, m), 2.53-2.47 (2H, 2×d, J7.3, rotamers), 2.33-2.16 (1H, m), 2.13-1.38 (6H, m), 1.75-1.51 (4H, m), 1.30-1.16 (2H, m); MS (ESI) 361.2 (M+H)⁺.

### Example 279: Synthesis of 1-(2-((1R,4R)-2,5-diazabicyclo[2.2.1]heptan-2-yl)-6,7-dihydrothiazolo[5,4-c]pyridin-5(4H)-yl)-2-cyclopentylethan-1-one

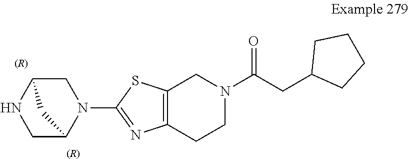

Prepared using tert-butyl (1R,4R)-2,5-diazabicyclo[2.2.1]heptane-2-carboxylate

¹H-NMR: (400 MHz, DMSO-d₆, mixture of rotamers) 4.54-4.45 (2H, m), 4.30-4.24 (1H, m), 3.76-3.66 (2H, m), 3.66-3.60 (1H, m), 3.45-3.40 (1H, m), 3.11-3.06 (1H, m), 2.89-2.84 (2H, m), 2.61-2.55 (2H, m), 2.44-2.36 (2H, m), 2.21-2.08 (1H, m), 1.81-1.68 (3H, m), 1.67-1.41 (5H, m), 1.18-1.05 (2H, m); MS (ESI) 347.2 (M+H)⁺.

### Example 280: Synthesis of 1-(2-(3,9-diazabicyclo[3.3.1]nonan-3-yl)-6,7-dihydrothiazolo[5,4-c]pyridin-5(4H)-yl)-2-cyclopentylethan-1-one

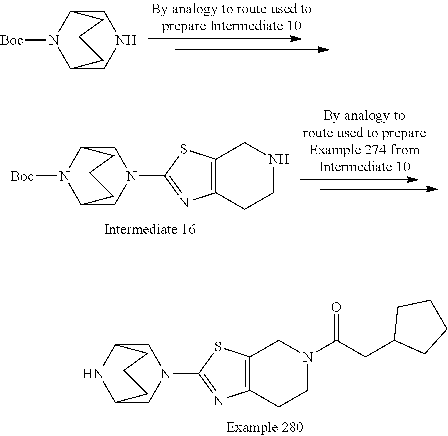 [SMILES: CC(C)(C)OC(=O)N1C2CCCC1CNC2 ; CC(C)(C)OC(=O)N1C2CCCC1CN(c1nc3c(s1)CNCC3)C2 ; O=C(CC1CCCC1)N1CCc2nc(N3CC4CCCC(C3)N4)sc2C1]

Prepared by conversion of tert-butyl 3,9-diazabicyclo[3.3.1]nonane-9-carboxylate into tert-butyl 3-(4,5,6,7-tetrahydrothiazolo[5,4-c]pyridin-2-yl)-3,9-diazabicyclo[3.3.1]nonane-9-carboxylate (Intermediate 16) by an analogy to the route described for preparation of Intermediate 10 and then conversion of Intermediate 16 into 1-(2-(3,9-diazabicyclo[3.3.1]nonan-3-yl)-6,7-dihydrothiazolo[5,4-c]pyridin-5(4H)-yl)-2-cyclopentylethan-1-one (Example 280) according to the route used to prepare Example 274 but using 2-cyclopentylacetic acid instead of 3,3-dimethylbutanoic acid. ¹H-NMR: δ_{H} (400 MHz, DMSO-d₆, mixture of rotamers) 4.55-4.44 (2H, m), 3.76-3.67 (2H, m), 3.67-3.60 (2H, m), 3.28-3.17 (2H, m), 3.12-3.02 (2H, m), 2.64-2.54 (2H, m), 2.48-2.34 (2H, m), 2.22-2.08 (1H, m), 1.91-1.85 (1H, m), 1.85-1.64 (6H, m), 1.64-1.43 (5H, m), 2.18-2.05 (2H, m); MS (ESI) 375.2 (M+H)⁺.

### Example 281: Synthesis of 1-(2-(3,8-diazabicyclo[3.2.1]octan-8-yl)-6,7-dihydrothiazolo[4,5-c]pyridin-5(4H)-yl)-2-cyclopentylethan-1-one

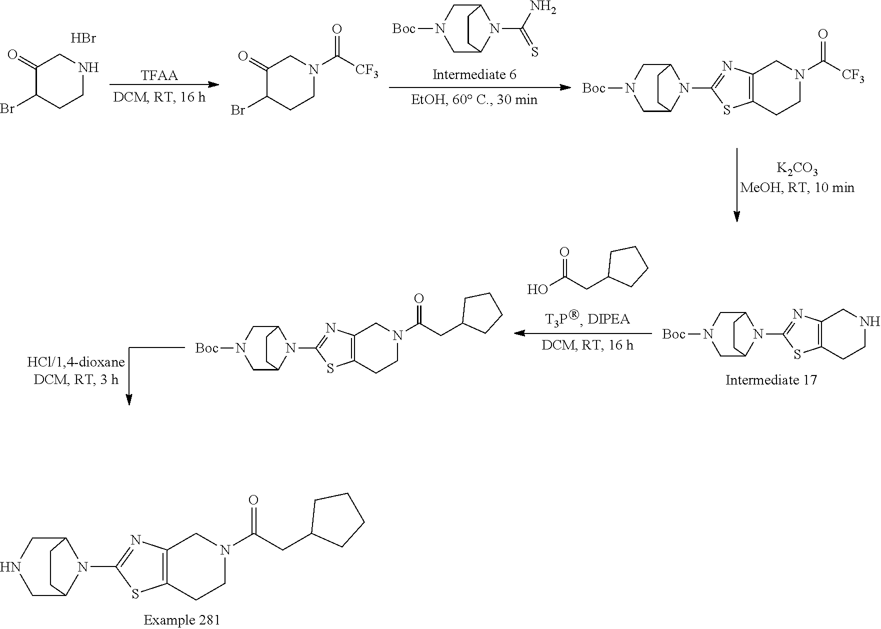

To a stirred solution of 4-bromopiperidin-3-one hydrobromide (2.50 g, 9.65 mmol) in DCM (30 mL) at 0° C. was added trifluoroacetic anhydride (2.68 mL, 19.3 mmol); the reaction mixture was then brought to room temperature. After 16 h the mixture was poured into ice-cold water and extracted twice with DCM. The combined organic extracts were washed successively with saturated NaHCO₃ aq and brine, dried (Na₂SO₄), and evaporated to afford 4-bromo-1-(2,2,2-trifluoroacetyl) piperidin-3-one (2.0 g, 76%) as a brown solid.

To a stirred solution of 4-bromo-1-(2,2,2-trifluoroacetyl) piperidin-3-one (2.0 g, 7.30 mmol) in ethanol (20 mL) at room temperature was added tert-butyl 8-carbamothioyl-3,8-diazabicyclo[3.2.1]octane-3-carboxylate (Intermediate 6, 1.39 g, 5.11 mmol). The reaction mixture was stirred at 60° C. for 30 min and then evaporated. The resulting residue was processed by flash column chromatography (0 to 70% EtOAc/petroleum ether eluent) to afford tert-butyl 8-(5-(2,2,2-trifluoroacetyl)-4,5,6,7-tetrahydrothiazolo[4,5-c]pyridin-2-yl)-3,8-diazabicyclo[3.2.1]octane-3-carboxylate (1.6 g, 70%) as an off-white solid.

To a stirred solution of tert-butyl 8-(5-(2,2,2-trifluoroacetyl)-4,5,6,7-tetrahydrothiazolo[4,5-c]pyridin-2-yl)-3,8-diazabicyclo[3.2.1]octane-3-carboxylate (1.6 g, 3.6 mmol) in methanol (15 mL) and water (3 mL) at room temperature was added potassium carbonate (0.991 g, 7.17 mmol). After 10 min the reaction mixture was concentrated to a residue that was diluted with water and extracted with 10% MeOH in DCM. The combined organic extracts were washed successively with water and brine, dried (Na₂SO₄), and evaporated to afford tert-butyl 8-(4,5,6,7-tetrahydrothiazolo[4,5-c]pyridin-2-yl)-3,8-diazabicyclo[3.2.1]octane-3-carboxylate (Intermediate 17, 1.3 g, quant.) as a brown solid.

To a stirred solution of tert-butyl 8-(4,5,6,7-tetrahydrothiazolo[4,5-c]pyridin-2-yl)-3,8-diazabicyclo[3.2.1]octane-3-carboxylate (Intermediate 17, 250 mg, 0.713 mmol) in DCM (10 mL) were added 2-cyclopentylacetic acid (137 mg, 1.07 mmol), n-propylphosphonic acid anhydride, cyclic trimer (50% in EtOAc; 0.454 mL, 1.43 mmol) and DIPEA (0.580 mL, 3.57 mmol). After 3 h the reaction mixture was diluted with water and extracted with DCM. The combined organic extracts were washed with brine, dried (Na₂SO₄), and evaporated. The resulting residue was processed by flash column chromatography (0 to 30% EtOAc/petroleum ether eluent) to afford tert-butyl 8-(5-(2-cyclopentylacetyl)-4,5,6,7-tetrahydrothiazolo[4,5-c]pyridin-2-yl)-3,8-diazabicyclo[3.2.1]octane-3-carboxylate (120 mg, 37%) as an off-white solid.

To a solution of tert-butyl 8-(5-(2-cyclopentylacetyl)-4,5,6,7-tetrahydrothiazolo[4,5-c]pyridin-2-yl)-3,8-diazabicyclo[3.2.1]octane-3-carboxylate (120 mg, 0.261 mmol) in DCM (10 mL) at 0° C. was added HCl (4M in 1,4-dioxane; 0.130 mL, 0.520 mmol); the reaction mixture was then brought to room temperature. After 3 h the mixture was evaporated to give a residue that was processed by reversed-phase preparative HPLC using TFA as a buffer. Pure fractions were combined and evaporated to obtain a solid (TFA salt). This was further processed by reversed-phase preparative HPLC using ammonium bicarbonate as a buffer to afford 1-(2-(3,8-diazabicyclo[3.2.1]octan-8-yl)-6,7-dihydrothiazolo[4,5-c]pyridin-5(4H)-yl)-2-cyclopentylethan-1-one (25.5 mg, 27%, Example 281) as an off-white solid. ¹H-NMR: δ_{H} (400 MHz, CD₃OD, mixture of rotamers) 4.62-4.60 (2H, m), 4.18-4.12 (2H, m), 3.89-384 (2H, m), 3.14-3.11 (2H, m), 2.75-2.60 (4H, m), 2.60-2.45 (2H, m), 2.33-2.15 (1H, m), 2.15-1.98 (4H, m), 1.95-1.75 (2H, m), 1.75-1.45 (4H, m), 1.30-1.15 (2H, m); MS (ESI) 361.2 (M+H)⁺.

### Example 282: Synthesis of 1-(2-(3,8-diazabicyclo[3.2.1]octan-8-yl)-6,7-dihydrothiazolo[4,5-c]pyridin-5(4H)-yl)-3,3-dimethylbutan-1-one

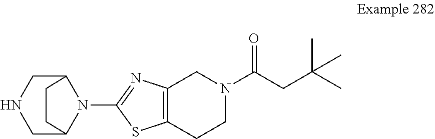 [SMILES: CC(C)(C)CC(=O)N1CCc2sc(N3C4CCC3CNC4)nc2C1]

Prepared by the route described for Example 281 but using 3,3-dimethylbutanoic acid instead of 2-cyclopentylacetic acid for reaction with Intermediate 17. ¹H-NMR: δ_{H} (400 MHz, DMSO-de, mixture of rotamers) 4.56-4.49 (2H, 2×s, rotamers), 4.02-3.96 (2H, m), 3.75 (2H, t, J 5.7), 2.94 (2H, app. d, J 12.2), 2.63-2.50 (4H, m), 2.32-2.28 (2H, 2×s, rotamers), 1.94-1.81 (4H, m), 1.01-0.97 (9H, 2×s, rotamers); MS (ESI) 349.2 (M+H)⁺.

### Example 283: Synthesis of 2-cyclopentyl-1-(2-(7-hydroxy-3,9-diazabicyclo[3.3.1]nonan-9-yl)-6,7-dihydrothiazolo[5,4-c]pyridin-5(4H)-yl)ethan-1-one

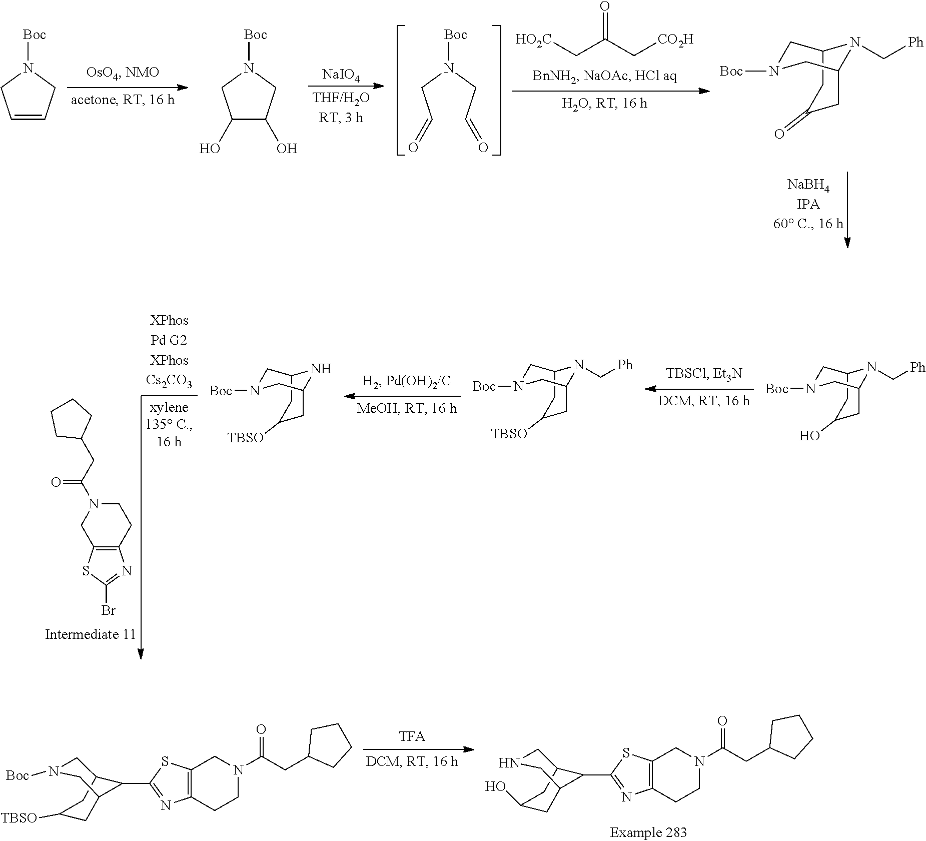

To a stirred solution of NMO (6.92 g, 59.1 mmol) in acetone (50 mL) and water (50 mL) was added osmium tetroxide (4 wt % in water, 10.8 mL, 1.70 mmol) at 0° C. and stirred for 30 min. To this tert-butyl 2,5-dihydro-1H-pyrrole-1-carboxylate (10.0 g, 59.1 mmol) in acetone (10 mL) was added and the resulting mixture was stirred at room temperature for 16 h. After completion, the reaction mixture was quenched with sodium bisulphite and concentrated to remove acetone. The mixture was acidified with dilute sulphuric acid (25 mL), diluted with EtOAc (25 mL), and filtered through a pad of Celite®. The filtrate was extracted with EtOAc (100 mL×3). The combined organic extracts were washed with brine (100 mL), dried (Na₂SO₄), filtered and concentrated under reduced pressure to afford crude tert-butyl 3,4-dihydroxypyrrolidine-1-carboxylate (9.8 g, 81%) as a purple liquid.

To a stirred solution of sodium periodate (1.05 g, 4.92 mmol) in water (10 mL) and THF (25 mL) was added tert-butyl 3,4-dihydroxypyrrolidine-1-carboxylate (1.00 g, 4.92 mmol) in THF (10 mL) at 0° C. The resulting mixture was stirred at room temperature for 3 h. After completion of the reaction, the mixture was filtered through a pad of Celite®, washing with THF (10 mL). The filtrate was concentrated to remove THF and the aqueous solution phase containing the crude tert-butyl bis(2-oxoethyl) carbamate was taken directly to the next step.

To a stirred solution of tert-butyl bis(2-oxoethyl) carbamate (ca. 86 mmol) in water (100 mL) at 0° C. was added sodium acetate (7.14 g, 87.0 mmol) and 3-oxopentanedioic acid (12.6 g, 86.1 mmol). The resulting mixture was stirred at 0° C. for 30 min. To this, a solution of benzylamine (9.23 g, 86.1 mmol) in 3M HCl (57.4 mL, 172 mmol) was added dropwise and stirred at room temperature for 16 hours. After completion, the reaction mixture was basified with K₂CO₃ solution (100 mL), diluted with 10% MeOH in DCM (25 mL) and filtered through a pad of Celite®. The filtrate was extracted with 10% MeOH in DCM (400 mL×3). The combined organic extracts were washed with brine (200 mL), dried (Na₂SO₄), filtered and concentrated under reduced pressure to give crude material that was processed by a flash column chromatography (0-60% EtOAc/petroleum ether eluent), affording tert-butyl 9-benzyl-7-oxo-3,9-diazabicyclo[3.3.1]nonane-3-carboxylate (13 g, 45%) as an off-white solid.

To a stirred solution of tert-butyl 9-benzyl-7-oxo-3,9-diazabicyclo[3.3.1]nonane-3-carboxylate (500 mg, 1.51 mmol) in 2-propanol (10 mL) at 0° C. was added NaBH₄ (343 mg, 9.08 mmol) and the resulting mixture was heated at 60° C. for 16 h. Afterwards, the reaction mixture was concentrated, diluted with ice-cold water (10 mL) and extracted with EtOAc (15 mL×3). The combined organic extracts were washed with brine (15 mL), dried (Na₂SO₄), filtered and concentrated under reduced pressure to afford tert-butyl 9-benzyl-7-hydroxy-3,9-diazabicyclo[3.3.1]nonane-3-carboxylate (490 mg, 95%) as a yellow oil.

To a stirred solution of tert-butyl 9-benzyl-7-hydroxy-3,9-diazabicyclo[3.3.1]nonane-3-carboxylate (1.60 g, 4.81 mmol) in DCM (10 mL) at 0° C. were added Et₃N (2.01 mL, 14.4 mmol), TBDMSCl (2.90 g, 19.2 mmol) and DMAP (59 mg, 0.48 mmol) and the resulting mixture was stirred at room temperature for 16 h. Afterwards, the mixture was quenched with 10% aqueous NaHCO₃ solution (20 mL), diluted with water (10 mL) and extracted with DCM (25 mL×3). The combined organic extracts were washed with brine (25 mL), dried (Na₂SO₄), filtered, and concentrated under reduced pressure to give crude material that was processed by flash column chromatography (0-30% EtOAc/petroleum ether eluent), affording tert-butyl 9-benzyl-7-((tert-butyldimethylsilyl)oxy)-3,9-diazabicyclo[3.3.1]nonane-3-carboxylate (1.68 g, 78%) as a yellow oil.

To a stirred solution of tert-butyl 9-benzyl-7-((tert-butyldimethylsilyl)oxy)-3,9-diazabicyclo[3.3.1]nonane-3-carboxylate (1.60 g, 3.58 mmol) in MeOH (15 mL) was added palladium hydroxide on carbon (20% dry basis) (0.45 g) under nitrogen atmosphere. The reaction mixture was stirred under a hydrogen gas bladder at room temperature for 16 h. After completion, the reaction mixture was filtered through a pad of Celite®, washing with MeOH (200 mL). The filtrate was concentrated under reduced pressure to give crude material that was processed by flash column chromatography (0-10% MeOH/DCM eluent), affording tert-butyl 7-((tert-butyldimethylsilyl)oxy)-3,9-diazabicyclo[3.3.1]nonane-3-carboxylate (900 mg, 70%) as an pale yellow semi solid.

To a stirred solution of 1-(2-bromo-6,7-dihydrothiazolo[5,4-c]pyridin-5(4H)-yl)-2-cyclopentylethan-1-one (Intermediate 11, 90.0 mg, 0.273 mmol) in xylene (10 mL) were added a solution of tert-butyl 7-((tert-butyldimethylsilyl)oxy)-3,9-diazabicyclo[3.3.1]nonane-3-carboxylate (107 mg, 0.300 mmol) in xylene (10 mL), Cs₂CO₃ (267 mg, 0.819 mmol), XPhos (26.1 mg, 0.0548 mmol) and XPhos Pd G2 (21.5 mg, 0.0273 mmol) under continuous bubbling with nitrogen. The resulting mixture was heated at 135° C. After 16 h the reaction mixture was cooled, diluted with DCM and filtered through a pad of Celite®. The filtrate was evaporated and the resulting residue processed by flash column chromatography (0 to 30% EtOAc/petroleum ether eluent) to afford tert-butyl 7-((tert-butyldimethylsilyl)oxy)-9-(5-(2-cyclopentylacetyl)-4,5,6,7-tetrahydrothiazolo[5,4-c]pyridin-2-yl)-3,9-diazabicyclo[3.3.1]nonane-3-carboxylate (140 mg, 85%).

To a stirred solution of tert-butyl (1R,5S)-7-((tert-butyldimethylsilyl)oxy)-9-(5-(2-cyclopentylacetyl)-4,5,6,7-tetrahydrothiazolo[5,4-c]pyridin-2-yl)-3,9-diazabicyclo[3.3.1]nonane-3-carboxylate (140 mg, 0.231 mmol) in DCM (4 mL) at 0° C. was added TFA (0.090 mL, 1.03 mmol); the reaction mixture was then brought to room temperature. After 16 h the mixture was evaporated to give a residue that was processed by reversed-phase preparative HPLC using ammonium bicarbonate as a buffer. Pure fractions were lyophilized to afford 2-cyclopentyl-1-(2-(7-hydroxy-3,9-diazabicyclo[3.3.1]nonan-9-yl)-6,7-dihydrothiazolo[5,4-c]pyridin-5(4H)-yl)ethan-1-one (8 mg, 9%, Example 283) as an off-white solid. ¹H-NMR: δ_{H} (400 MHz, DMSO-d₆, mixture of rotamers) 4.52-4.48 (2H, m), 4.03-3.96 (2H, m), 3.76-3.67 (3H, m), 2.92-2.80 (4H, m), 2.63-2.50 (2H, m), 2.44-2.35 (2H, m), 2.21-2.07 (1H, m), 1.82-1.40 (10H, m), 1.20-1.03 (3H, m); MS (ESI) 391.3 (M+H)⁺.

### Example 284: Synthesis of (S)-2-cyclopentyl-1-(2-(2-(hydroxymethyl)piperazin-1-yl)-6,7-dihydrothiazolo[5,4-c]pyridin-5(4H)-yl)ethan-1-one TFA Salt

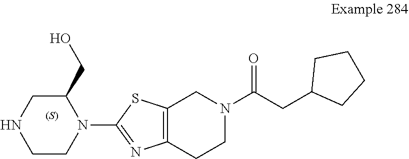

Prepared according to the method of Example 275, using tert-butyl (S)-3-(hydroxymethyl)piperazine-1-carboxylate instead of tert-butyl (R)-3-(hydroxymethyl)piperazine-1-carboxylate.

¹H-NMR: δ_{H} (400 MHz, DMSO-d₆, mixture of rotamers) 9.11 (1H, br d, J 10.8), 8.35 (1H, br q, J 10.5), 4.55-4.50 (2H, m), 4.10-4.03 (1H, m), 3.89-3.81 (1H, m), 3.80-3.74 (1H, m), 3.74-3.67 (3H, m), 3.50-3.39 (2H, m), 3.33-3.26 (1H, m), 3.26-3.15 (1H, m), 3.14-3.00 (1H, m), 2.62-2.53 (2H, m), 2.44-2.37 (2H, m), 2.22-2.08 (1H, m), 1.81-1.65 (2H, m), 1.65-1.41 (4H, m), 1.18-1.04 (2H, m); MS (ESI) 365.2 (M+H)⁺.

### Examples 285 and 286: Synthesis of 2-(3,8-diazabicyclo[3.2.1]octan-8-yl)-5-((4-fluorobenzyl)sulfonyl)-4,5,6,7-tetrahydrothiazolo[5,4-c]pyridine and 2-(3-ethyl-3,8-diazabicyclo[3.2.1]octan-8-yl)-5-((4-fluorobenzyl)sulfonyl)-4,5,6,7-tetrahydrothiazolo[5,4-c]pyridine

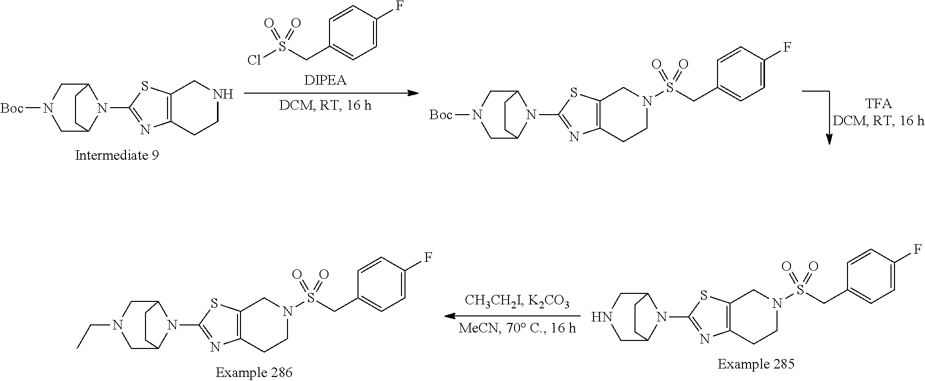 [SMILES: CC(C)(C)OC(=O)N1CC2CCC(C1)N2c1nc2c(s1)CNCC2 ; O=S(=O)(Cl)Cc1ccc(F)cc1 ; CC(C)(C)OC(=O)N1CC2CCC(C1)N2c1nc2c(s1)CN(S(=O)(=O)Cc1ccc(F)cc1)CC2 ; CCN1CC2CCC(C1)N2c1nc2c(s1)CN(S(=O)(=O)Cc1ccc(F)cc1)CC2 ; O=S(=O)(Cc1ccc(F)cc1)N1CCc2nc(N3C4CCC3CNC4)sc2C1]

To a stirred solution of tert-butyl 8-(4,5,6,7-tetrahydrothiazolo[5,4-c]pyridin-2-yl)-3,8-diazabicyclo[3.2.1]octane-3-carboxylate (Intermediate 9, 400 mg, 1.14 mmol) in DCM (5 mL) at room temperature were added (4-fluorophenyl) methanesulfonyl chloride (357 mg, 1.71 mmol) and DIPEA (443 mg, 3.42 mmol). After 16 h the mixture was diluted with water (20 mL) and extracted with 10% MeOH in DCM (10 mL×2). The combined organic extracts were washed with brine (20 mL), dried (Na₂SO₄), and evaporated to give a residue that was processed by flash column chromatography (0 to 100% EtOAc/petroleum ether eluent) to afford tert-butyl 8-(5-((4-fluorobenzyl)sulfonyl)-4,5,6,7-tetrahydrothiazolo[5,4-c]pyridin-2-yl)-3,8-diazabicyclo[3.2.1]octane-3-carboxylate (300 mg, 50%) as an off-white solid.

To a stirred solution of tert-butyl 8-(5-((4-fluorobenzyl)sulfonyl)-4,5,6,7-tetrahydrothiazolo[5,4-c]pyridin-2-yl)-3,8-diazabicyclo[3.2.1]octane-3-carboxylate (50 mg, 0.096 mmol) in DCM (3 mL) at room temperature was added TFA (0.022 mL, 0.25 mmol). After 16 h the reaction mixture was diluted with sat. NaHCO₃ aq (20 mL) and extracted with 10% MeOH in DCM (10 mL×2). The combined organic extracts were washed with brine (20 mL), dried (Na₂SO₄), and evaporated to give a residue that was processed by reversed-phase preparative HPLC using ammonium bicarbonate as a buffer. The pure fractions were lyophilized to afford 2-(3,8-diazabicyclo[3.2.1]octan-8-yl)-5-((4-fluorobenzyl)sulfonyl)-4,5,6,7-tetrahydrothiazolo[5,4-c]pyridine (12 mg, 30%, Example 285) as an off-white solid. ¹H-NMR: δ_{H} (400 MHz, CD₃OD) 7.47-7.41 (2H, m), 7.13-7.05 (2H, m), 4.42 (2H, s), 4.24 (2H, s), 4.16-4.10 (2H, m), 3.54 (2H, t, J 5.8), 3.13 (2H, app. d, J 12.3), 2.68 (2H, app. d, J 12.5), 2.62-2.55 (2H, m), 2.15-1.97 (4H, m); MS (ESI) 423.1 (M+H)⁺.

To a stirred solution of 2-(3,8-diazabicyclo[3.2.1]octan-8-yl)-5-((4-fluorobenzyl)sulfonyl)-4,5,6,7-tetrahydrothiazolo[5,4-c]pyridine (100 mg, 0.237 mmol) in acetonitrile (3 mL) were added iodoethane (25.8 mg, 0.166 mmol) and potassium carbonate (98 mg, 0.71 mmol) at room temperature. The resulting mixture was heated at 70° C. for 16 h. After completion, the reaction mixture was diluted with water (20 mL) and extracted with EtOAc (10 mL×2). The combined organic extracts were washed with brine (20 mL), dried (Na₂SO₄), and evaporated to give a residue (140 mg) that was processed by reversed-phase preparative HPLC using ammonium bicarbonate as a buffer. The pure fractions were lyophilized to obtain 2-(3-ethyl-3,8-diazabicyclo[3.2.1]octan-8-yl)-5-((4-fluorobenzyl)sulfonyl)-4,5,6,7-tetrahydrothiazolo[5,4-c]pyridine (5 mg, 7%, Example 286) as an off-white solid. ¹H-NMR: δ_{H} (400 MHz, DMSO-d₆) 7.45-7.41 (2H, m), 7.22-7.15 (2H, m), 4.50 (2H, s), 4.23 (2H, s), 4.16-4.08 (2H, m), 3.43 (2H, t, J 5.8), 2.66 (2H, dd, J 11.2 and 2.1), 2.58-2.51 (2H, m), 2.35-2.25 (4H, m), 1.91-1.78 (4H, m), 0.97 (3H, t, J 7.2); MS (ESI) 451.1 (M+H)⁺.

### Example 287: Synthesis of 5-((4-fluorobenzyl)sulfonyl)-2-(piperazin-1-yl)-4,5,6,7-tetrahydrothiazolo[5,4-c]pyridine

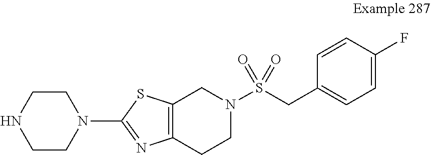 [SMILES: O=S(=O)(Cc1ccc(F)cc1)N1CCc2nc(N3CCNCC3)sc2C1]

Prepared as shown for Example 285 but by reaction of tert-butyl 4-(4,5,6,7-tetrahydrothiazolo[5,4-c]pyridin-2-yl)piperazine-1-carboxylate (Intermediate 10) with phenylmethanesulfonyl chloride in the first step in place of the reaction of Intermediate 9 with (4-fluorophenyl) methanesulfonyl chloride. ¹H-NMR: δ_{H} (400 MHz, DMSO-d₆) 7.44-7.40 (2H, m), 7.22-7.15 (2H, m), 4.49 (2H, s), 4.24 (2H, s), 3.43 (2H, t, J 5.7), 3.29-3.22 (4H, m), 2.80-2.74 (4H, m), 2.58-2.54 (2H, m); MS (ESI) 397.1 (M+H)⁺.

### Example 288: Synthesis 5-(benzylsulfonyl)-2-(3,8-diazabicyclo[3.2.1]octan-8-yl)-4,5,6,7-tetrahydrothiazolo[5,4-c]pyridine

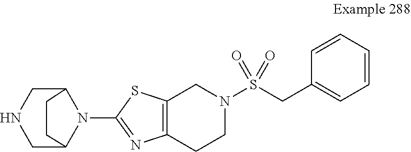 [SMILES: O=S(=O)(Cc1ccccc1)N1CCc2nc(N3C4CCC3CNC4)sc2C1]

Prepared as schematised for Example 285 but using phenylmethanesulfonyl chloride in the first step in place of (4-fluorophenyl) methanesulfonyl chloride. ¹H-NMR: δ_{H} (400 MHz, DMSO-d₆) 7.43-7.33 (5H, m), 4.48 (2H, s), 4.23 (2H, s), 4.02-3.95 (2H, m), 3.42 (2H, t, J5.8), 2.94 (2H, app. d, J 12.2), 2.62-2.53 (4H, m) 1.94-1.82 (4H, m); MS (ESI) 405.1 (M+H)⁺.

### Example 289: Synthesis of 5-(benzylsulfonyl)-2-(3-ethyl-3,8-diazabicyclo[3.2.1]octan-8-yl)-4,5,6,7-tetrahydrothiazolo[5,4-c]pyridine

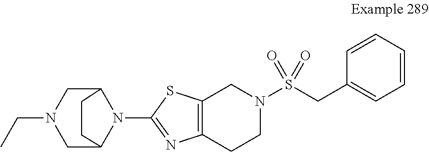 [SMILES: CCN1CC2CCC(C1)N2c1nc2c(s1)CN(S(=O)(=O)Cc1ccccc1)CC2]

Prepared as schematised for Example 286 but using phenylmethanesulfonyl chloride in the first step in place of (4-fluorophenyl) methanesulfonyl chloride. ¹H-NMR: δ_{H} (400 MHZ, CD₃OD) 7.44-7.32 (5H, m), 4.43 (2H, s), 4.23-4.16 (4H, m), 3.51 (2H, t, J5.8), 2.80-2.72 (2H, m), 2.58-2.50 (2H, m), 2.47-2.33 (4H, m), 2.07-1.93 (4H, m), 1.07 (3H, t, J 7.2); MS (ESI) 433.1 (M+H)⁺.

### Example 290: Synthesis of 2-cyclopentyl-1-(2-(3-isopropyl-3,8-diazabicyclo[3.2.1]octan-8-yl)-6,7-dihydrothiazolo[5,4-c]pyridin-5(4H)-yl)ethan-1-one

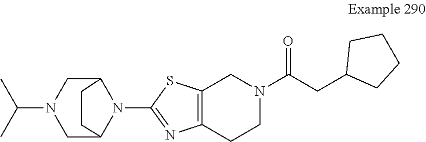 [SMILES: CC(C)N1CC2CCC(C1)N2c1nc2c(s1)CN(C(=O)CC1CCCC1)CC2]

Prepared by alkylation of Example 1 following the procedure described for conversion of Example 285 into Example 286 but using 2-iodopropane in place of iodoethane. ¹H-NMR: OH (400 MHZ, DMSO-d₆, mixture of rotamers) 4.52-4.48 (2H, m), 4.16-4.08 (2H, m), 3.76-3.67 (2H, m), 2.62-2.45 (7H, m), 2.45-2.34 (2H, m), 2.22-2.06 (1H, m), 1.87-1.67 (6H, m), 1.65-1.41 (4H, m), 1.19-1.05 (2H, m), 0.93 (6H, d, J6.5); MS (ESI) 403.2 (M+H)⁺.

### Example 291: Synthesis of 2-cyclopentyl-1-(2-(3-ethyl-3,8-diazabicyclo[3.2.1]octan-8-yl)-6,7-dihydrothiazolo[5,4-c]pyridin-5(4H)-yl)ethan-1-one

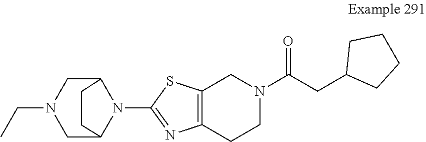 [SMILES: CCN1CC2CCC(C1)N2c1nc2c(s1)CN(C(=O)CC1CCCC1)CC2]

Prepared by alkylation of Example 1 with idoethane following the procedure described for conversion of Example 285 into Example 286. ¹H-NMR: δ_{H} (400 MHZ, DMSO-d₆, mixture of rotamers) 4.52-4.48 (2H, m), 4.16-4.08 (2H, m), 3.76-3.67 (2H, m), 2.69-2.62 (2H, m), 2.62-2.53 (2H, m), 2.45-2.36 (2H, m), 2.32-2.24 (4H, m), 2.21-2.07 (1H, m), 1.91-1.67 (6H, m), 1.65-1.40 (4H, m), 1.18-1.07 (2H, m), 0.96 (3H, t, J 7.2); MS (ESI) 389.2 (M+H)⁺.

### Example 292: Synthesis of 2-cyclopentyl-1-(2-(3-propyl-3,8-diazabicyclo[3.2.1]octan-8-yl)-6,7-dihydrothiazolo[5,4-c]pyridin-5(4H)-yl)ethan-1-one

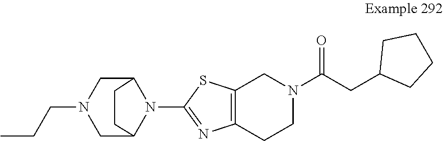 [SMILES: CCCN1CC2CCC(C1)N2c1nc2c(s1)CN(C(=O)CC1CCCC1)CC2]

Prepared by alkylation of Example 1 following the procedure described for conversion of Example 285 into Example 286 but using 1-iodopropane in place of iodoethane. ¹H-NMR: OH (400 MHZ, DMSO-d₆, mixture of rotamers) 4.52-4.48 (2H, m), 4.16-4.08 (2H, m), 3.76-3.67 (2H, m), 2.68-2.53 (4H, m), 2.45-2.35 (2H, m), 2.33-2.25 (2H, m), 2.25-2.08 (3H, m), 1.91-1.68 (6H, m), 1.65-1.34 (6H, m), 1.18-1.05 (2H, m) 0.85 (3H, t, J7.3); MS (ESI) 403.2 (M+H)⁺.

### Examples 293 and 294: Synthesis of 2-(3,8-diazabicyclo[3.2.1]octan-8-yl)-N—((S)-chroman-4-yl)-6,7-dihydrothiazolo[5,4-c]pyridine-5(4H)-carboxamide and N—((S)-chroman-4-yl)-2-(3-ethyl-3,8-diazabicyclo[3.2.1]octan-8-yl)-6,7-dihydrothiazolo[5,4-c]pyridine-5(4H)-carboxamide

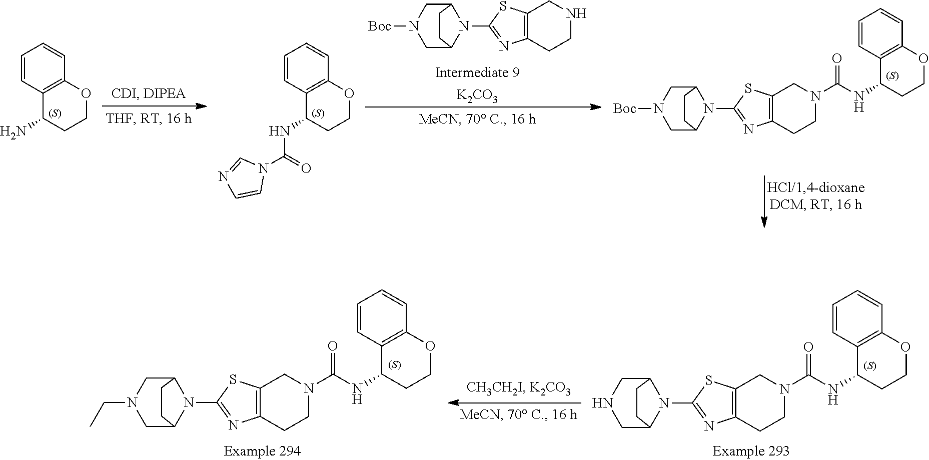

To a stirred solution of (S)-chroman-4-amine (2.00 g, 13.4 mmol) in THF (20 mL) at room temperature were added CDI (4.35 g, 26.8 mmol) and DIPEA (11.6 mL, 67.0 mmol). The reaction mixture was stirred at room temperature for 16 h. Afterwards, the reaction mixture was diluted with water (100 mL) and extracted with EtOAc (50 mL×2). The combined organic extracts were washed with brine (100 mL), dried (Na₂SO₄), filtered, and concentrated under reduced pressure to afford (S)—N-(chroman-4-yl)-1H-imidazole-1-carboxamide (2.5 g, 77%) as an off-white solid.

To a stirred solution of (S)—N-(chroman-4-yl)-1H-imidazole-1-carboxamide (300 mg, 1.23 mmol) in acetonitrile (5 mL) were added tert-butyl 8-(4,5,6,7-tetrahydrothiazolo[5,4-c]pyridin-2-yl)-3,8-diazabicyclo[3.2.1]octane-3-carboxylate (648 mg, 1.85 mmol) and K₂CO₃ (170 mg, 1.23 mmol). The resulting mixture was heated at 70° C. After 16 h the reaction mixture was cooled, diluted with water (20 mL) and extracted with DCM (5 mL×3). The combined organic extracts were washed with brine (20 mL), dried (Na₂SO₄), and evaporated to give a residue that was processed by flash column chromatography (0 to 100% EtOAc/petroleum ether eluent) to afford tert-butyl 8-(5-(((S)-chroman-4-yl) carbamoyl)-4,5,6,7-tetrahydrothiazolo[5,4-c]pyridin-2-yl)-3,8-diazabicyclo[3.2.1]octane-3-carboxylate (280 mg, 43%) as an off-white solid.

To a stirred solution of tert-butyl 8-(5-(((S)-chroman-4-yl) carbamoyl)-4,5,6,7-tetrahydrothiazolo[5,4-c]pyridin-2-yl)-3,8-diazabicyclo[3.2.1]octane-3-carboxylate (450 mg, 0.856 mmol) in DCM (3 mL) was added 4M HCl in dioxane (0.64 mL, 2.6 mmol) at 0° C.; the mixture was then brought to room temperature. After 16 h the reaction mixture was evaporated. The residue was treated with saturated NaHCO₃ aq (10 mL) and extracted with EtOAc (10 mL×2). The combined organic extracts were washed with brine (20 mL), dried (Na₂SO₄) and evaporated. The crude product (300 mg) was processed by reversed-phase preparative HPLC using ammonium bicarbonate as buffer. The combined purified fractions were lyophilized to afford 2-(3,8-diazabicyclo[3.2.1]octan-8-yl)-N—((S)-chroman-4-yl)-6,7-dihydrothiazolo[5,4-c]pyridine-5(4H)-carboxamide (18 mg, 5%, Example 293) as an off-white solid. ¹H-NMR: δ_{H} (400 MHZ, DMSO-d₆) 7.19-7.09 (2H, m), 7.02 (1H, d, J 8.0), 6.86 (1H, td, J 8.0 and 1.1), 6.76 (1H, dd, J 8.0 and 1.0), 4.93 (1H, app. q, J 6.8), 4.48-4.34 (2H, AB m), 4.28-4.14 (2H, m), 4.02-3.95 (2H, m), 3.72-3.58 (2H, m), 2.96-2.89 (2H, m), 2.55-2.43 (4H, m), 2.07-1.81 (6H, m); MS (ESI) 426.2 (M+H)⁺.

To a stirred solution of 2-(3,8-diazabicyclo[3.2.1]octan-8-yl)-N—((S)-chroman-4-yl)-6,7-dihydrothiazolo[5,4-c]pyridine-5(4H)-carboxamide (230 mg, 0.540 mmol) in acetonitrile (5 mL) were added ethyl iodide (76 mg, 0.49 mmol) and potassium carbonate (308 mg, 2.70 mmol). The resulting mixture was heated at 70° C. After 16 h the reaction mixture was cooled, diluted with water (10 mL) and extracted with EtOAc (20 mL×2). The combined organic extracts were washed with brine (10 mL), dried (Na₂SO₄) and evaporated. The crude product was processed by reversed-phase preparative HPLC using ammonium bicarbonate as buffer. The combined purified fractions were lyophilized to afford N—((S)-chroman-4-yl)-2-(3-ethyl-3,8-diazabicyclo[3.2.1]octan-8-yl)-6,7-dihydrothiazolo[5,4-c]pyridine-5(4H)-carboxamide (25 mg, 10%, Example 294) as an off-white solid. ¹H-NMR: δ_{H} (400 MHZ, DMSO-d₆, 80° C.) 7.24-7.17 (1H, m), 7.15-7.08 (1H, m), 6.88-6.82 (1H, m), 6.80-6.71 (2H, m), 5.00-4.93 (1H, m), 4.49-4.36 (2H, m), 4.29-4.09 (4H, m), 3.73-3.60 (2H, m), 2.70-2.64 (2H, m), 2.57-2.52 (2H, m), 2.38-2.32 (4H, m), 2.10-1.94 (2H, m), 1.94-1.82 (4H, m), 0.99 (3H, t, J7.1); MS (ESI) 454.2 (M+H)⁺.

### Example 295: Synthesis of N—((R)-chroman-4-yl)-2-(3-ethyl-3,8-diazabicyclo[3.2.1]octan-8-yl)-6,7-dihydrothiazolo[5,4-c]pyridine-5(4H)-carboxamide

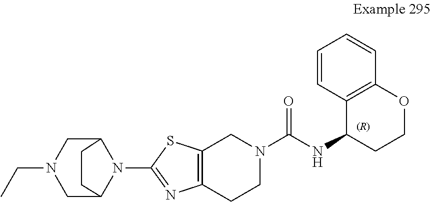

Prepared as described for Example 294 but starting from (R)-chroman-4-amine instead of (S)-chroman-4-amine. ¹H-NMR: δ_{H} (400 MHZ, DMSO-d₆) 7.18-7.10 (2H, m), 7.02-6.98 (1H, m), 6.88-6.84 (1H, m), 6.77-6.74 (1H, m), 4.98-4.89 (1H, m), 4.48-4.34 (2H, m), 4.28-4.11 (4H, m), 3.73-3.59 (2H, m), 2.69-2.64 (2H, m), 2.62-2.52 (2H, m), 2.35-2.25 (4H, m), 2.05-1.75 (6H, m), 0.97 (3H, t, J7.1); MS (ESI) 454.2 (M+H)⁺.

### Examples 296 and 297: Synthesis of (S)—N-(chroman-4-yl)-2-(piperazin-1-yl)-6,7-dihydrothiazolo[5,4-c]pyridine-5(4H)-carboxamide and (S)—N-(chroman-4-yl)-2-(4-ethylpiperazin-1-yl)-6,7-dihydrothiazolo[5,4-c]pyridine-5(4H)-carboxamide

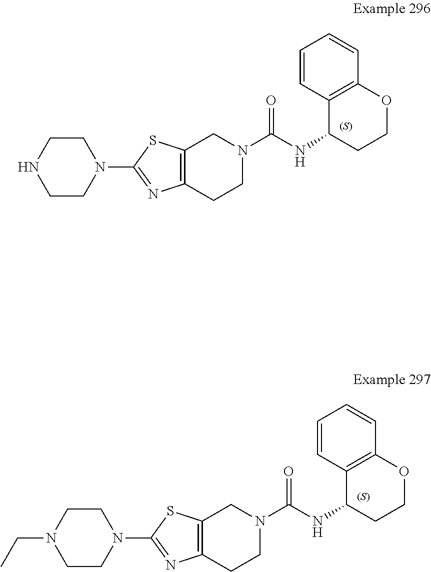

Prepared as shown for Example 293 and Example 294 but using tert-butyl 4-(4,5,6,7-tetrahydrothiazolo[5,4-c]pyridin-2-yl) piperazine-1-carboxylate (Intermediate 10) in the second step in place of Intermediate 9. Example 296. ¹H-NMR: δ_{H} (400 MHZ, DMSO-d₆) 7.18-7.09 (2H, m), 7.04-6.97 (1H, m), 6.89-6.83 (1H, m), 6.77-6.73 (1H, m), 4.96-4.90 (1H, m), 4.48-4.34 (2H, m), 4.28-4.13 (2H, m), 3.71-3.57 (2H, m), 3.28-3.22 (4H, m), 2.80-2.73 (4H, m), 2.62-2.52 (2H, m), 2.06-1.86 (2H, m); MS (ESI) 400.2 (M+H)⁺.

Example 297. ¹H-NMR: δ_{H} (400 MHZ, DMSO-d₆) 7.18-7.10 (2H, m), 7.01 (1H, d, J8.1), 6.86 (1H, td, J7.6 and 1.2), 6.76 (1H, dd, J 8.1 and 1.1), 4.97-4.89 (1H, m), 4.49-4.34 (2H, m), 4.28-4.13 (2H, m), 3.72-3.57 (2H, m), 3.36-3.30 (4H, m), 2.62-2.52 (2H, m), 2.48-2.42 (4H, m), 2.37 (2H, q, J7.1), 2.06-1.88 (2H, m), 1.02 (3H, t, J7.1); MS (ESI) 428.1 (M+H)⁺.

### Example 298: Synthesis of 2-(4-fluorophenoxy)-1-(2-(3-isopropyl-3,8-diazabicyclo[3.2.1]octan-8-yl)-6,7-dihydrothiazolo[5,4-c]pyridin-5(4H)-yl)ethan-1-one

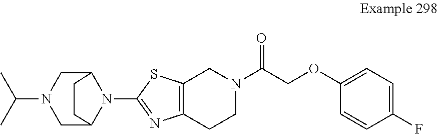 [SMILES: CC(C)N1CC2CCC(C1)N2c1nc2c(s1)CN(C(=O)COc1ccc(F)cc1)CC2]

Prepared by alkylation of Example 21 following the procedure described for conversion of Example 285 into Example 286 but using 2-iodopropane in place of iodoethane. ¹H-NMR: OH (400 MHZ, DMSO-d₆, mixture of rotamers) 7.15-7.09 (2H, m), 6.98-6.92 (2H, m), 4.94-4.85 (2H, m), 4.57-4.49 (2H, m), 4.18-4.09 (2H, m), 3.78-3.67 (2H, m), 2.70-2.43 (7H, m), 1.86-1.78 (4H, m), 0.93 (6H, d, J 6.5); MS (ESI) 445.2 (M+H)⁺.

### Example 299:1-(2-(4-ethylpiperazin-1-yl)-6,7-dihydrothiazolo[5,4-c]pyridin-5(4H)-yl)-3,3-dimethylbutan-1-one

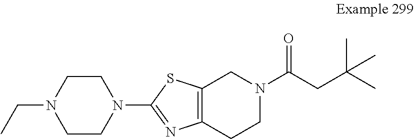 [SMILES: CCN1CCN(c2nc3c(s2)CN(C(=O)CC(C)(C)C)CC3)CC1]

Prepared by alkylation of Example 274 following the procedure described for conversion of Example 285 into Example 286 using iodoethane. ¹H-NMR: δ_{H} (400 MHZ, CD₃OD, mixture of rotamers) 4.68-4.60 (2H, m), 3.92-3.86 (2H, m), 3.50-3.45 (4H, m), 2.74-2.58 (6H, m), 2.52 (2H, q, J 7.3), 2.44-2.41 (2H, 2×s, rotamers), 1.16 (3H, t, J 7.2), 1.10-1.02 (9H, 2×s, rotamers); MS (ESI) 351.2 (M+H)⁺.

### Example 300: Synthesis of 2-(3-ethyl-3,8-diazabicyclo[3.2.1]octan-8-yl)-N-(4-fluorobenzyl)-6,7-dihydrothiazolo[5,4-c]pyridine-5(4H)-carboxamide

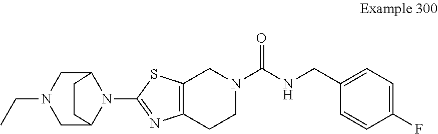 [SMILES: CCN1CC2CCC(C1)N2c1nc2c(s1)CN(C(=O)NCc1ccc(F)cc1)CC2]

Prepared by alkylation of Example 55 following the procedure described for conversion of Example 285 into Example 286 using iodoethane. ¹H-NMR: δ_{H} (400 MHZ, CD₃OD) 7.34-7.29 (2H, m), 7.06-7.01 (2H, m), 4.51-4.44 (2H, m), 4.38-4.35 (2H, m), 4.22-4.18 (2H, m), 3.74 (2H, t, J 5.8), 2.79-2.73 (2H, m), 2.67-2.62 (2H, m), 2.44-2.35 (4H, m), 2.08-1.92 (4H, m), 1.06 (3H, t, J7.2); MS (ESI) 430.2 (M+H)⁺.

### Example 301: Synthesis of 2-(4-fluorophenoxy)-1-(2-(3-methyl-3,8-diazabicyclo[3.2.1]octan-8-yl)-6,7-dihydrothiazolo[5,4-c]pyridin-5(4H)-yl)ethan-1-one

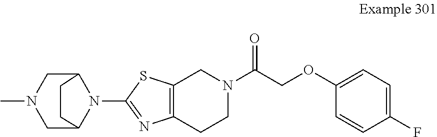 [SMILES: CN1CC2CCC(C1)N2c1nc2c(s1)CN(C(=O)COc1ccc(F)cc1)CC2]

Prepared by alkylation of Example 21 following the procedure described for conversion of Example 285 into Example 286 but using iodomethane in place of iodoethane. ¹H-NMR: δ_{H} (400 MHz, DMSO-d₆, mixture of rotamers) 7.15-7.07 (2H, m), 6.99-6.91 (2H, m), 4.94-4.85 (2H, m), 4.58-4.47 (2H, m), 4.15-4.08 (2H, m), 3.79-3.68 (2H, m), 2.70-2.52 (4H, m), 2.31-2.26 (2H, m), 2.13 (3H, s), 1.92-1.79 (4H, m); MS (ESI) 417.2 (M+H)⁺.

### Example 302: Synthesis of 1-(2-(3-ethyl-3,8-diazabicyclo[3.2.1]octan-8-yl)-6,7-dihydrothiazolo[5,4-c]pyridin-5(4H)-yl)-2-(4-fluorophenoxy)ethan-1-one

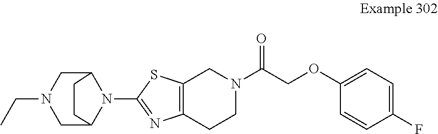 [SMILES: CCN1CC2CCC(C1)N2c1nc2c(s1)CN(C(=O)COc1ccc(F)cc1)CC2]

Prepared by alkylation of Example 21 following the procedure described for conversion of Example 285 into Example 286 using iodoethane. ¹H-NMR: δ_{H} (400 MHZ, DMSO-d₆, mixture of rotamers) 7.14-7.08 (2H, m), 6.98-6.92 (2H, m), 4.91-4.86 (2H, m), 4.56-4.50 (2H, m), 4.17-4.09 (2H, m), 3.78-3.68 (2H, m), 2.72-2.61 (4H, m), 2.34-2.24 (4H, m), 1.91-1.78 (4H, m), 0.96 (3H, t, J 7.2); MS (ESI) 431.2 (M+H)⁺.

### Example 303: Synthesis of 2-(4-fluorophenoxy)-1-(2-(3-propyl-3,8-diazabicyclo[3.2.1]octan-8-yl)-6,7-dihydrothiazolo[5,4-c]pyridin-5(4H)-yl)ethan-1-one

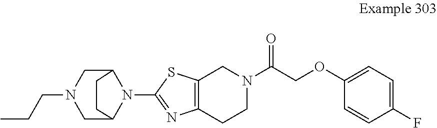 [SMILES: CCCN1CC2CCC(C1)N2c1nc2c(s1)CN(C(=O)COc1ccc(F)cc1)CC2]

Prepared by alkylation of Example 21 following the procedure described for conversion of Example 285 into Example 286 but using 1-iodopropane in place of iodoethane. ¹H-NMR: OH (400 MHZ, DMSO-d₆, mixture of rotamers) 7.14-7.08 (2H, m) 6.98-6.92 (2H, m), 4.91-4.86 (2H, m) 4.56-4.50 (2H, m) 4.15-4.09 (2H, m), 3.78-3.68 (2H, m), 2.69-2.54 (4H, m), 2.32-2.25 (2H, m), 2.20 (2H, t, J7.1), 1.91-1.78 (4H, m), 1.40 (2H, sextet, J 7.1) 0.85 (3H, t, J 7.2); MS (ESI) 445.2 (M+H)⁺.

### Example 304: Synthesis of 2-(4-ethylpiperazin-1-yl)-5-((4-fluorobenzyl)sulfonyl)-4,5,6,7-tetrahydrothiazolo[5,4-c]pyridine

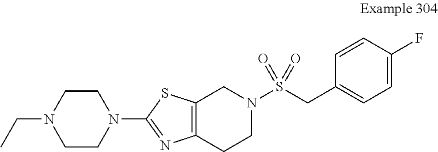 [SMILES: CCN1CCN(c2nc3c(s2)CN(S(=O)(=O)Cc2ccc(F)cc2)CC3)CC1]

Prepared by alkylation of Example 287 following the procedure described for conversion of Example 285 into Example 286 using iodoethane. ¹H-NMR: δ_{H} (400 MHZ, DMSO-d₆) 7.45-7.40 (2H, m), 7.22-7.16 (2H, m), 4.49 (2H, s), 4.24 (2H, s), 3.43 (2H, t, J 5.8), 3.37-3.31 (4H, m), 2.58-2.53 (2H, m), 2.47-2.42 (4H, m), 2.37 (2H, q, J 7.2), 1.02 (3H, t, J 7.2); MS (ESI) 425.1 (M+H)⁺.

### Example 305: Synthesis of 2-cyclopentyl-1-(2-(6-methylpyridin-3-yl)-6,7-dihydrothiazolo[5,4-c]pyridin-5(4H)-yl)ethan-1-one

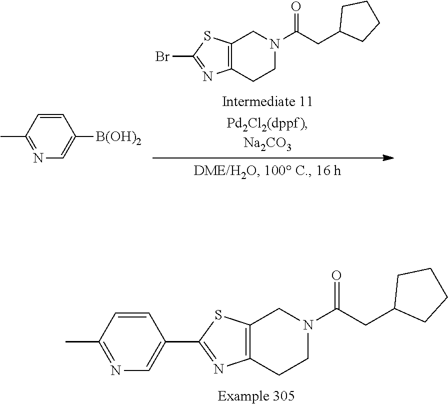

To a stirred suspension of 1-(2-bromo-6,7-dihydrothiazolo[5,4-c]pyridin-5(4H)-yl)-2-cyclopentylethan-1-one (Intermediate 11; 1 equiv.), (6-methylpyridin-3-yl) boronic acid (1 equiv.) and Na₂CO₃ (2 equiv.) in a mixture of DME (4 vol.) and water (1 vol.) was added PdCl₂ (dppf)·CH₂Cl₂ (0.1 equiv.) under continuous bubbling with nitrogen. The resulting mixture was then heated at 100° C. After 16 h the reaction mixture was cooled and filtered through a pad of Celite®, washing with EtOAc. The filtrate was concentrated to give a residue that was processed by reversed-phase preparative HPLC using ammonium bicarbonate as a buffer, affording 2-cyclopentyl-1-(2-(6-methylpyridin-3-yl)-6,7-dihydrothiazolo[5,4-c]pyridin-5(4H)-yl)ethan-1-one as an off-white solid.

¹H-NMR: δ_{H} (400 MHZ, DMSO-d₆, mixture of rotamers) 8.97-8.93 (1H, m), 8.16-8.10 (1H, m), 7.39 (1H, d, J 8.0), 4.83-4.77 (2H, m), 3.86-3.79 (2H, m), 2.94-2.78 (2H, m), 2.53 (3H, s), 2.50-2.40 (2H, m), 2.23-2.07 (1H, m), 1.85-1.65 (2H, m), 1.65-1.40 (4H, m), 1.18-1.07 (2H, m); MS (ESI) 342.1 (M+H)⁺.

### Example 306: Synthesis of 2-cyclopentyl-1-(2-(5-methylpyridin-3-yl)-6,7-dihydrothiazolo[5,4-c]pyridin-5(4H)-yl)ethan-1-one

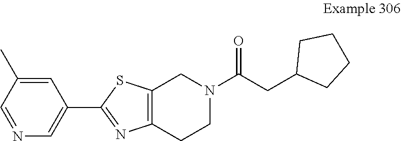 [SMILES: Cc1cncc(-c2nc3c(s2)CN(C(=O)CC2CCCC2)CC3)c1]

Prepared as Example 305 but using 3-methyl-5-(4,4,5,5-tetramethyl-1,3,2-dioxaborolan-2-yl)pyridine in place of (6-methylpyridin-3-yl) boronic acid in the reaction with Intermediate 11. ¹H-NMR: δ_{H} (400 MHZ, DMSO-d₆, mixture of rotamers) 8.88 (1H, app. s), 8.50 (1H, app. s), 8.07 (1H, app. s), 4.85-4.78 (2H, m), 3.87-3.80 (2H, m), 2.96-2.79 (2H, m), 2.50-2.40 (2H, m), 2.39 (3H, s), 2.25-2.10 (1H, m), 1.85-1.65 (2H, m), 1.65-1.40 (4H, m), 1.18-1.07 (2H, m); MS (ESI) 342.1 (M+H)⁺.

### Biological Assays

### Cell Culture

HEK 293 and MDCK cells (Public Health England, Cell Culture Collections) were maintained in Dulbecco's Modified Eagle's Medium (DMEM) supplemented with 10% (v/v) fetal bovine serum (FBS) (Seralab), 2 mM L-glutamine and 100 U/mL penicillin streptomycin cocktail (ThermoFisher); termed complete media. Clonal HEK 293 cell lines were maintained in complete media supplemented with 0.6 mg/ml G418 (Enzo Life Sciences). Mouse inner medullary collecting duct (m-IMCD3) cells [American Type Culture Collection (ATCC)] were maintained in media consisting of DMEM/Hams F-12 50/50 Mix (DMEM F12; Corning) supplemented with penicillin streptomycin cocktail (100 U/mL) and FBS (10% v/v) (DMEM F12-10% FBS).

### Experiment 1: Measurement of Modulation of Enzyme Activity by PDE4 Long Form Activators of the Present Invention Using Full-Length Human PDE4 Isoforms; Long Forms PDE4D5, PDE4C3, PDE4B1 and Short Form PDE4B2

(Marchmont, R. J. and Houslay, M. D. Biochem. J. 187: 381-92, 1980)

### Exogenous Expression of Long Form PDE4 Enzymes and Stable Cell Line Generation

For transient transfection of exogenous PDE4 long isoforms, HEK 293 cells were transfected with pcDNA3.1 or pDEST™ PDE4 expression vectors using Lipofectamine LTX/Plus reagent (Invitrogen) as outlined by the manufacturer.

Where stable cell lines were generated the clonal isolates were expanded to obtain cell lines that stably expressed the full-length human PDE4 long isoforms and the full length human PDE4B2 short isoform. These were called the HEK-PDE4D5, HEK-PDE4B1 and HEK-PDE4B2 cell lines, respectively.

### Lysate Preparation (Using PDE4D5 as a Typical Example)

HEK-PDE4D5 cells were seeded out in 100 mm plates and incubated at 37° C. in an atmosphere of 5% CO₂, 95% air. Cell lysates were prepared using KHEM buffer [50 mM KCl, 10 mM EGTA, 50 mM HEPES (pH 7.2), 1.92 mM MgCl₂].

To prepare the cell lysates, the 100 mm plates containing the cells were placed on ice and washed with ice-cold PBS (phosphate buffered saline, pH 7.4). KHEM buffer (500 μL) was added to the cells. Cells were then scraped off the plate and triturated using a needle (BD Microlance™ 0.8, 40 mm). The lysed cells were then centrifuged at 2000 rpm for 10 minutes to remove cell debris and the supernatant (cell lysate containing recombinant PDE4D5) was transferred to a fresh tube and kept on ice.

### Cytosol Fraction Preparation (Using PDE4D5 as a Typical Example)

The cell lysate containing recombinant PDE4D5 was transferred into a centrifuge tube and placed into an ultracentrifuge (BECKMAN COULTER) and spun at high speed (100,000 g) for 30 minutes at 4° C. The cytosol fraction was then collected and its protein amount determined using a BCA protein assay.

### PDE Assay—(Using PDE4D5 as a Typical Example)

PDE assays were performed in thin-walled V-bottomed 96-well plates. The assays were performed at a final concentration of 10 mM Tris/5 mM MgCl₂ plus PDE4D5 cell lysate cytosol fraction, containing over-expressed PDE4D5, with and without test compound. The lysate/compound mix were incubated together for 15 min at room temperature on an orbital shaker prior to addition of [³H] cAMP (final concentration 1 μM [³H] cAMP; Perkin Elmer) to a final volume of 50 μL per reaction. The reactions were then incubated for 10 minutes at 30° C., terminated by heating for 2 min at 95 and allowed to cool. Snake venom (12.5 μL of 1 mg/mL; Crotalus atrox, Sigma) was then added and the plates were agitated and incubated for a further 15 minutes at 30° C. Dowex ion exchange resin (Sigma, chloride form, 200-400 mesh; 200 μL; prepared as a 1:1 Dowex:water stock, thoroughly re-suspended and diluted 2:1 with ethanol) was then added to each well and the plates incubated for 15 min at room temperature on an orbital shaker ensuring sufficient agitation for resin suspension (550 RPM). The reaction mixture was then transferred to a 96 well filter plate (Millipore; 0.45 μm pore size) and filtered into a receiving 96-well plate to remove the dowex suspension. 30 μL of the filtered solution was then transferred to the wells of an Opti-plate (Perkin Elmer) 96 well assay plate and 120 μL of Microscint 40 scintillation fluid was added. The plate was then placed on an orbital shaker for 10 min at high speed (900 RPM) to mix the sample with scintillation fluid prior to quantitation using a plate based scintillation counter (Top-Count).

The % increase in counts in the presence of test compound at a particular concentration indicates the % increase in enzyme activity at that concentration.

Data are shown in Table 2.

Experiment 2: Reduction of cAMP Levels in m-IMCD3 Cell Culture Treated with PDE4 Long Form Activators

Mouse Inner Medullary Collecting Duct (m-IMCD3) cells have been shown to express PDE4A, PDE4B, PDE4C and PDE4D long isoforms (Hansen et al., EMBO reports e54315 | 2022, see FIG. 4). Activation of intracellular PDE4 long forms leads to a reduction of both intracellular cAMP and externalised cAMP (Omar et al., PNAS 116: 13320-13329, 2019). This reduction may be measured in the cell culture media supernatant of 3D cystic cell cultures, such as in the m-IMCD3 cell cyst suppression assay described in Experiment 3.

Following the completion of the m-IMCD3 cell cyst suppression assay (Experiment 3), the cAMP level in the assay media from individual or pooled assay wells was measured using a cAMP ELISA kit (Enzo Life Sciences) or a LANCE Ultra cAMP kit (PerkinElmer) according to the manufacturer's instructions.

Treatment with PDE4 long form activators reduced cAMP levels in PGE2-stimulated m-IMCD3 cell culture. Data are shown in Table 3.

Experiment 3: Inhibition of In Vitro Cyst Formation in m-IMCD3 Cells Treated with PDE4 Long Form Activators

The mouse Inner Medullary Collecting Duct cell line (m-IMCD3) spontaneously forms cystic spheroids in 3D culture with a Type 1 collagen/Matrigel extracellular matrix. This process can be stimulated with agents which raise intracellular cAMP and is used as an in vitro model for the formation of cystic structures in the kidneys of patients with ADPKD.

Rat Collagen I (Fisher Scientific) is prepared on ice by neutralising with 1M NaOH and diluting with a 2× volume of DMEM/F12+10% FBS. This is mixed 1:1.1 with ice cold Matrigel (Corning) for coating plates (coating mix), and 1:0.93 with Matrigel for cell plating (plating mix).

The assay is conducted in the wells of a 96-well plate culture dish using a total volume of 130 μL of collagen/Matrigel/DMEM F12-10% FBS suspension in growth media for each matrix plug per individual well. Initially, 30 μL of collagen/Matrigel/DMEM F12-10% FBS (coating mix) is added into the well of a 96 well plate and the collagen is set to a gel by incubating at 37° C. for at least 15 minutes. A second layer of 100 μL collagen/Matrigel suspension (plating mix) containing m-IMCD3 cells (2.75×106 m-IMCD3 cells per 96-well plate) is layered over the coating mix and the collagen/Matrigel/cell mix again set to a gel by incubating at 37° C. Cell cultures are maintained at 37° C. in an atmosphere of 5% CO₂, 95% air.

Between 18 and 24 h after plating, test compound(s) as DMSO stock solutions [0.1% (v/v) final DMSO concentration] and PGE2 (100 nM final concentration) in DMEM F12-10% FBS are added in quadruplicate wells per condition. Media, together with the test compound and PGE2 are replenished after 2 or 3 days. After 6 days of culture z stack images of the wells are captured using the Nikon Eclipse Ti2-E microscope. Nikon General Analysis software is used to measure the following parameters in each well: the mean cyst area, the number of cysts, and the total cyst area.

PDE4 long form activators inhibited in vitro cyst formation in m-IMCD3 cells. Data are shown in Table 4 as mean cyst area (%), compared to 100% for (DMSO+PGE2) and 0% for DMSO control.

### Experiment 4: Inhibition of Proliferation of LNCaP Human Prostate Cancer Cells

In this study, the potential utility of PDE4 long form activators in the treatment of prostate cancer is studied using the LNCaP human prostate cancer cell line. The experiments are carried out according to the method described by Henderson et al. (Henderson, D. J. P., Byrne, A., Dulla, K., Jenster, G., Hoffmann, R., Baillie, G. S., Houslay, M. D. Br. J. Cancer 110: 1278-1287, 2014).

### LNCaP Cell Culture

Androgen-sensitive (AS) LNCaP cells are maintained in RPMI1640 supplemented with 10% FBS (Seralabs), 2 mM L-glutamine and 1,000U penicillin-streptomycin. LNCaP androgen-insensitive (AI) cells are generated by culturing the LNCaP-AS cells in RPMI1640 supplemented with 10% charcoal stripped FBS, 2 mM L-glutamine and 1,000U penicillin-streptomycin for a minimum of four weeks. All tissue culture reagents are from Life Technologies.

### Xcelligence (Roche) Proliferation Assay

Cell proliferation is measured as a function of changing electrical impedance. Values are represented by cell index number, a dimensionless unit of measurement representing the cell status, which increases as cells adhere to 96-well electrode plates and divide.

LNCaP AI/AS cells are plated at a density of 25,000 cells per well in a 96-well electrode plate (in triplicate), in the presence/absence of various concentrations of test compound.

Cell indices are measured every 10 minutes for up to 100 hours, analysed using RTCA software and normalised to the cell index of vehicle-treated cells (n=3).

PDE4 long form activators inhibited the proliferation of AS and AI LNCaP human prostate cancer cells.

Experiment 5: In vivo preclinical model of hyperparathyroidism: Inhibition of PTH-induced cAMP elevation in urine in the anaesthetised rat

Within the kidney, the binding of parathyroid hormone (PTH) to PTH receptors results in the Gₐₛ-mediated elevation of intracellular cAMP. This increase of intracellular cAMP results in extrusion of cAMP to the urine (Yates et al., J Clin Invest 81: 932-938, 1988). This experiment is based upon a modified Ellsworth-Howard assay (Kruse, K. and Kracht, U., European Journal of Pediatrics 146: 373-377, 1987) and conducted in anaesthetized rats. In this experiment, rats were anaesthetized using isoflurane and catheterised to allow the collection of urine from the bladder. After an initial stabilization period, test compounds were administered by i.v. infusion (modelled to steady state) from time 0-120 min. PTH challenge infusion (33 ug/kg/hour) was started after 60 min of test compound infusion and sustained for one hour (60-120 min). Urine collection was conducted in 30-minute periods. Urine cAMP levels were assessed by ELISA (R&D systems). Urine samples were prepared for analysis as per the manufacturer's instructions. A standard curve for cAMP was assayed for each experiment and samples were assessed using a standard dilution range of 1:2, 1:4, 1:8 and 1:16, ensuring that the resulting data remained on the linear portion of the standard curve.

Control animals treated with vehicle alone (no PTH) showed no increase from baseline in urinary cAMP concentrations over the course of the experiment. Urine cAMP concentration remained below 50,000 pmol/mL. Control animals treated with PTH infusion plus vehicle (PTH challenge) showed an increase from baseline in cAMP concentration in urine collected from 90 to 120 min.

Treatment with PDE4 long form activators suppressed the elevation in cAMP concentration in urine in response to PTH challenge.

TABLE 1
Small molecule PDE4 long form activators according to the present invention

| | | Mole- | |
|---|---|---|---|
| | | cular | |
| Com- | | weight | |
| pound | Chemical name | (Daltons) | Chemical structure |
| Example 1 | 1-(2-(3,8- diazabicyclo[3.2.1]octan-8-yl)- 6,7-dihydrothiazolo[5,4- c]pyridin-5(4H)-yl)-2- cyclopentylethan-1-one | 360 | 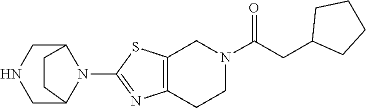 [SMILES: O=C(CC1CCCC1)N1CCc2nc(N3C4CCC3CNC4)sc2C1] |
| Example 2 | 1-(2-(3,8- diazabicyclo[3.2.1]octan-8-yl)- 6,7-dihydrothiazolo[5,4- c]pyridin-5(4H)-yl)-2-(4,4- difluorocyclohexyl)ethan-1-one | 410 | 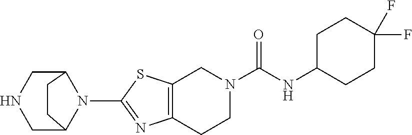 [SMILES: O=C(NC1CCC(F)(F)CC1)N1CCc2nc(N3C4CCC3CNC4)sc2C1] |
| Example 3 | 1-(2-(3,8- diazabicyclo[3.2.1]octan-8-yl)- 6,7-dihydrothiazolo[5,4- c]pyridin-5(4H)-yl)-2- (tetrahydro-2H-pyran-4- yl)ethan-1-one | 376 | 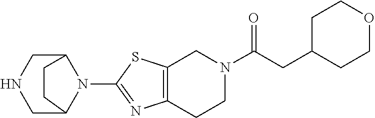 [SMILES: O=C(CC1CCOCC1)N1CCc2nc(N3C4CCC3CNC4)sc2C1] |
| Example 4 | 1-(2-(3,8-diazabicyclo[3.2.1]octan-8-yl)- 6,7-dihydrothiazolo[5,4- c]pyridin-5(4H)-yl)-2- isopropoxyethan-1-one | 350 | 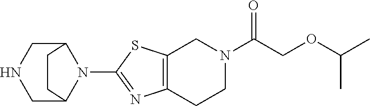 [SMILES: CC(C)OCC(=O)N1CCc2nc(N3C4CCC3CNC4)sc2C1] |
| Example 5 | 1-(2-(3,8-diazabicyclo[3.2.1]octan-8-yl)- 6,7-dihydrothiazolo[5,4- c]pyridin-5(4H)-yl)-3-phenylpropan-1-one | 382 | 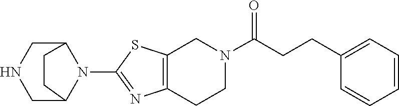 [SMILES: O=C(CCc1ccccc1)N1CCc2nc(N3C4CCC3CNC4)sc2C1] |
| Example 6 | 1-(2-(3,8-diazabicyclo[3.2.1]octan-8-yl)- 6,7-dihydrothiazolo[5,4- c]pyridin-5(4H)-yl)-2-phenoxyethan-1-one | 384 | 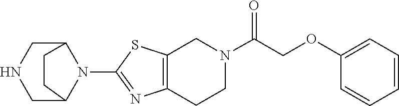 [SMILES: O=C(COc1ccccc1)N1CCc2nc(N3C4CCC3CNC4)sc2C1] |
| Example 7 | 4-(3-(2-(3,8-diazabicyclo[3.2.1]octan-8- yl)-6,7-dihydrothiazolo[5,4- c]pyridin-5(4H)-yl)-3-oxopropyl)benzonitrile | 407 | 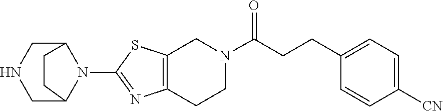 [SMILES: N#Cc1ccc(CCC(=O)N2CCc3nc(N4C5CCC4CNC5)sc3C2)cc1] |
| Example 8 | 1-(2-(3,8-diazabicyclo[3.2.1]octan-8-yl)- 6,7-dihydrothiazolo[5,4- c]pyridin-5(4H)-yl)-2-phenylethan-1-one | 368 | 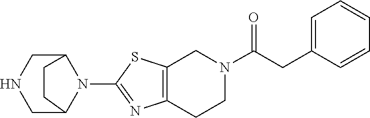 [SMILES: O=C(Cc1ccccc1)N1CCc2nc(N3C4CCC3CNC4)sc2C1] |
| Example 9 | 1-(2-(3,8-diazabicyclo[3.2.1]octan-8-yl)- 6,7-dihydrothiazolo[5,4- c]pyridin-5(4H)-yl)-3-methylbutan-1-one | 334 | 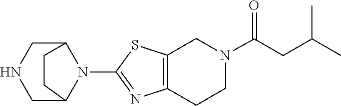 [SMILES: CC(C)CC(=O)N1CCc2nc(N3C4CCC3CNC4)sc2C1] |
| Example 10 | 1-(2-(3,8-diazabicyclo[3.2.1]octan-8-yl)- 6,7-dihydrothiazolo[5,4- c]pyridin-5(4H)-yl)-3,3-dimethylbutan-1-one | 348 | 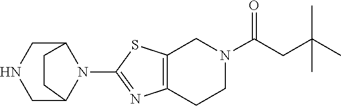 [SMILES: CC(C)(C)CC(=O)N1CCc2nc(N3C4CCC3CNC4)sc2C1] |
| Example 11 | 1-(2-(3,8-diazabicyclo[3.2.1]octan-8-yl)- 6,7-dihydrothiazolo[5,4- c]pyridin-5(4H)-yl)-2-cyclobutylethan-1- one | 346 | 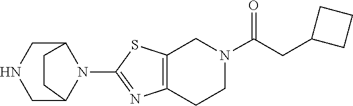 [SMILES: O=C(CC1CCC1)N1CCc2nc(N3C4CCC3CNC4)sc2C1] |
| Example 12 | 1-(2-(3,8-diazabicyclo[3.2.1]octan-8-yl)- 6,7-dihydrothiazolo[5,4- c]pyridin-5(4H)-yl)-2-(bicyclo[1.1.1]pentan-1- yl)ethan-1-one | 358 | 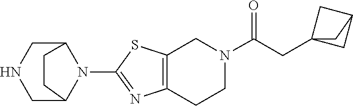 [SMILES: O=C(CC12CC(C1)C2)N1CCc2nc(N3C4CCC3CNC4)sc2C1] |
| Example 13 | 1-(2-(3,8-diazabicyclo[3.2.1]octan-8-yl)- 6,7-dihydrothiazolo[5,4- c]pyridin-5(4H)-yl)-2-cyclohexylethan-1-one | 374 | 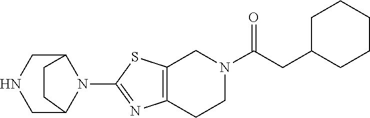 [SMILES: O=C(CC1CCCCC1)N1CCc2nc(N3C4CCC3CNC4)sc2C1] |
| Example 14 | (2-(3,8-diazabicyclo[3.2.1]octan-8-yl)- 6,7-dihydrothiazolo[5,4-c]pyridin-5(4H)-yl) (4,4-difluorocyclohexyl)methanone | 396 | 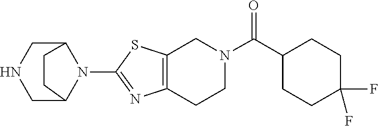 [SMILES: O=C(C1CCC(F)(F)CC1)N1CCc2nc(N3C4CCC3CNC4)sc2C1] |
| Example 15 | (2S)-1-(2-(3,8-diazabicyclo[3.2.1]octan-8-yl)- 6,7-dihydrothiazolo[5,4-c]pyridin-5(4H)-yl)- 2-methylbutan-1-one | 334 | 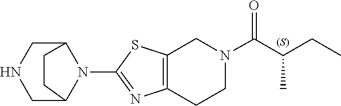 |
| Example 16 | (2R)-1-(2-(3,8-diazabicyclo[3.2.1]octan-8- yl)-6,7-dihydrothiazolo[5,4-c]pyridin-5(4H)-yl)- 2-methylbutan-1-one | 334 | 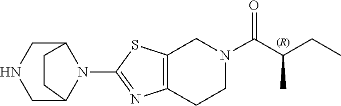 |
| Example 17 | 1-(2-(3,8-diazabicyclo[3.2.1]octan-8-yl)- 6,7-dihydrothiazolo[5,4-c]pyridin-5(4H)-yl)- 2,3-dimethylbutan-1-one | 348 | 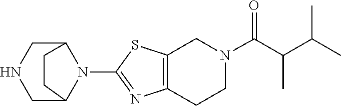 [SMILES: CC(C)C(C)C(=O)N1CCc2nc(N3C4CCC3CNC4)sc2C1] |
| Example 18 | (2S)-1-(2-(3,8-diazabicyclo[3.2.1]octan-8-yl)- 6,7-dihydrothiazolo[5,4-c]pyridin-5(4H)-yl)- 2,3-dimethylbutan-1-one | 348 | 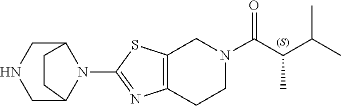 |
| Example 19 | (2R)-1-(2-(3,8-diazabicyclo[3.2.1]octan-8-yl)- 6,7-dihydrothiazolo[5,4-c]pyridin-5(4H)-yl)- 2,3-dimethylbutan-1-one | 348 | 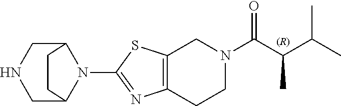 |
| Example 20 | 1-(2-(3,8-diazabicyclo[3.2.1]octan-8-yl)- 6,7-dihydrothiazolo[5,4-c]pyridin-5(4H)-yl)- 2-methylpropan-1-one | 320 | 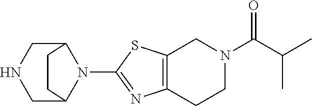 [SMILES: CC(C)C(=O)N1CCc2nc(N3C4CCC3CNC4)sc2C1] |
| Example 21 | 1-(2-(3,8-diazabicyclo[3.2.1]octan-8-yl)- 6,7-dihydrothiazolo[5,4-c]pyridin-5(4H)-yl)- 2-(4-fluorophenoxy)ethan-1-one | 402 | 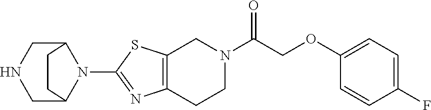 [SMILES: O=C(COc1ccc(F)cc1)N1CCc2nc(N3C4CCC3CNC4)sc2C1] |
| Example 22 | (2-(3,8-diazabicyclo[3.2.1]octan-8-yl)- 6,7-dihydrothiazolo[5,4-c]pyridin-5(4H)-yl) (cyclopentyl)methanone | 346 | 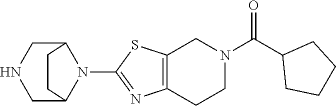 [SMILES: O=C(C1CCCC1)N1CCc2nc(N3C4CCC3CNC4)sc2C1] |
| Example 23 | 1-(2-(3,8-diazabicyclo[3.2.1]octan-8-yl)- 6,7-dihydrothiazolo[5,4-c]pyridin-5(4H)-yl)- 2-phenoxypropan-1-one | 398 | 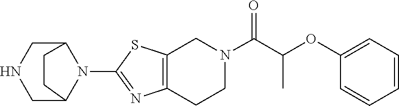 [SMILES: CC(Oc1ccccc1)C(=O)N1CCc2nc(N3C4CCC3CNC4)sc2C1] |
| Example 24 | 1-(2-(3,8-diazabicyclo[3.2.1]octan-8-yl)- 6,7-dihydrothiazolo[5,4-c]pyridin-5(4H)-yl)-2- cyclopentyl-2,2-difluoroethan-1-one | 396 | 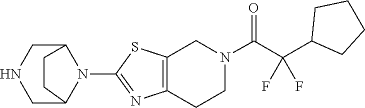 [SMILES: O=C(N1CCc2nc(N3C4CCC3CNC4)sc2C1)C(F)(F)C1CCCC1] |
| Example 25 | 1-(2-(3,8-diazabicyclo[3.2.1]octan-8-yl)- 6,7-dihydrothiazolo[5,4-c]pyridin-5(4H)-yl)- 2-(4-fluorophenyl)ethan-1-one | 386 | 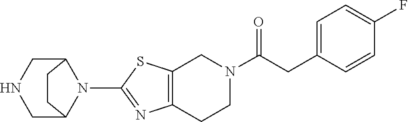 [SMILES: O=C(Cc1ccc(F)cc1)N1CCc2nc(N3C4CCC3CNC4)sc2C1] |
| Example 26 | 1-(2-(3,8-diazabicyclo[3.2.1]octan-8-yl)- 6,7-dihydrothiazolo[5,4-c]pyridin-5(4H)-yl)- 3,3,3-trifluoropropan-1-one | 360 | 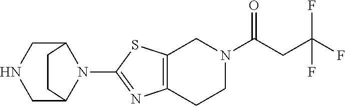 [SMILES: O=C(CC(F)(F)F)N1CCc2nc(N3C4CCC3CNC4)sc2C1] |
| Example 27 | 1-(2-(3,8-diazabicyclo[3.2.1]octan-8-yl)- 6,7-dihydrothiazolo[5,4-c]pyridin-5(4H)-yl)- 2-(1-methylcyclopentyl)ethan-1-one | 374 | 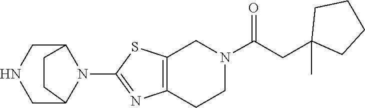 [SMILES: CC1(CC(=O)N2CCc3nc(N4C5CCC4CNC5)sc3C2)CCCC1] |
| Example 28 | 1-(2-(3,8-diazabicyclo[3.2.1]octan-8-yl)- 6,7-dihydrothiazolo[5,4-c]pyridin-5(4H)-yl)- 2-hydroxy-3-methylbutan-1-one | 350 | 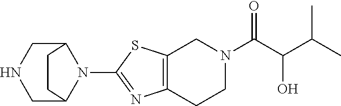 [SMILES: CC(C)C(O)C(=O)N1CCc2nc(N3C4CCC3CNC4)sc2C1] |
| Example 29 | (2-(3,8-diazabicyclo[3.2.1]octan-8-yl)- 6,7-dihydrothiazolo[5,4-c]pyridin-5(4H)-yl)(3,3- difluorocyclopentyl)methanone | 382 | 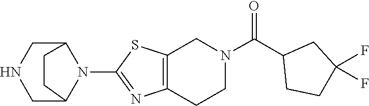 [SMILES: O=C(C1CCC(F)(F)C1)N1CCc2nc(N3C4CCC3CNC4)sc2C1] |
| Example 30 | 1-(2-(3,8-diazabicyclo[3.2.1]octan-8-yl)- 6,7-dihydrothiazolo[5,4-c]pyridin-5(4H)-yl)-2- methyl-2-phenoxypropan-1-one | 412 | 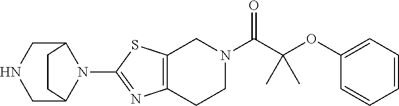 [SMILES: CC(C)(Oc1ccccc1)C(=O)N1CCc2nc(N3C4CCC3CNC4)sc2C1] |
| Example 31 | (2-(3,8-diazabicyclo[3.2.1]octan-8-yl)- 6,7-dihydrothiazolo[5,4-c]pyridin-5(4H)-yl)(1- phenoxycyclopropyl)methanone | 410 | 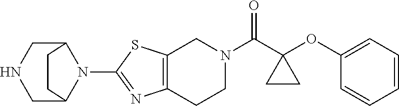 [SMILES: O=C(N1CCc2nc(N3C4CCC3CNC4)sc2C1)C1(Oc2ccccc2)CC1] |
| Example 32 | 1-(2-(3,8-diazabicyclo[3.2.1]octan-8-yl)- 6,7-dihydrothiazolo[5,4-c]pyridin-5(4H)-yl)- 3,3,3-trifluoro-2,2-dimethylpropan-1-one | 388 | 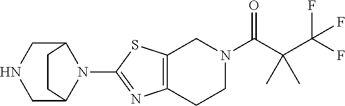 [SMILES: CC(C)(C(=O)N1CCc2nc(N3C4CCC3CNC4)sc2C1)C(F)(F)F] |
| Example 33 | (2-(3,8-diazabicyclo[3.2.1]octan-8-yl)- 6,7-dihydrothiazolo[5,4-c]pyridin-5(4H)-yl)(1- phenylcyclopropyl)methanone | 394 | 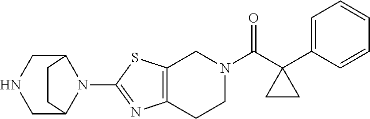 [SMILES: O=C(N1CCc2nc(N3C4CCC3CNC4)sc2C1)C1(c2ccccc2)CC1] |
| Example 34 | 1-(2-(3,8-diazabicyclo[3.2.1]octan-8-yl)- 6,7-dihydrothiazolo[5,4-c]pyridin-5(4H)-yl)-2- cyclopentyl-2-fluoroethan-1-one | 378 | 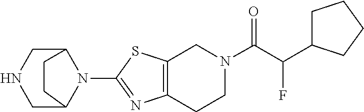 [SMILES: O=C(C(F)C1CCCC1)N1CCc2nc(N3C4CCC3CNC4)sc2C1] |
| Example 35 | 1-(2-(3,8-diazabicyclo[3.2.1]octan-8-yl)- 6,7-dihydrothiazolo[5,4-c]pyridin-5(4H)-yl)-2- (4,4-difluorocyclohexyl)-2-fluoroethan-1-one | 428 | 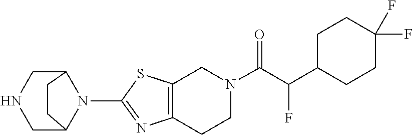 [SMILES: O=C(C(F)C1CCC(F)(F)CC1)N1CCc2nc(N3C4CCC3CNC4)sc2C1] |
| Example 36 | 1-(2-(3,8-diazabicyclo[3.2.1]octan-8-yl)- 6,7-dihydrothiazolo[5,4-c]pyridin-5(4H)-yl)-2,2- difluoro-2-phenylethan-1-one | 404 | 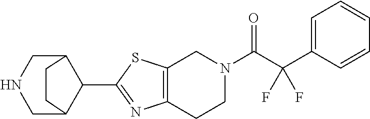 [SMILES: O=C(N1CCc2nc(C3C4CCC3CNC4)sc2C1)C(F)(F)c1ccccc1] |
| Example 37 | (2-(3,8-diazabicyclo[3.2.1]octan-8-yl)- 6,7-dihydrothiazolo[5,4-c]pyridin-5(4H)-yl)(2,2- dimethylcyclopentyl)methanone | 374 | 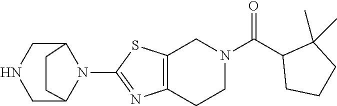 [SMILES: CC1(C)CCCC1C(=O)N1CCc2nc(N3C4CCC3CNC4)sc2C1] |
| Example 38 | (2-(3,8-diazabicyclo[3.2.1]octan-8-yl)- 6,7-dihydrothiazolo[5,4-c]pyridin-5(4H)-yl)(3,3- dimethylcyclopentyl)methanone | 374 | 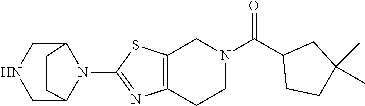 [SMILES: CC1(C)CCC(C(=O)N2CCc3nc(N4C5CCC4CNC5)sc3C2)C1] |
| Example 50 | 2-(3,8-diazabicyclo[3.2.1]octan-8- yl)-N-cyclopentyl-6,7-dihydrothiazolo [5,4-c]pyridine-5(4H)-carboxamide | 361 | 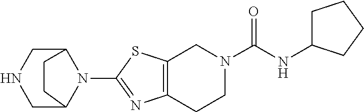 [SMILES: O=C(NC1CCCC1)N1CCc2nc(N3C4CCC3CNC4)sc2C1] |
| Example 51 | 2-(3,8-diazabicyclo[3.2.1]octan-8-yl)- N-isopropyl-6,7-dihydrothiazolo[5,4- c]pyridine-5(4H)-carboxamide | 335 | 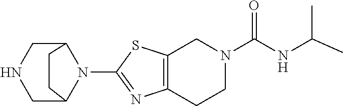 |
| Example 52 | 2-(3,8-diazabicyclo[3.2.1]octan-8-yl)- N-cyclohexyl-6,7-dihydrothiazolo[5,4-c] pyridine-5(4H)-carboxamide | 375 | 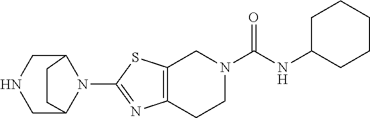 |
| Example 53 | 2-(3,8-diazabicyclo[3.2.1]octan-8- yl)-N-cycloheptyl-6,7-dihydrothiazolo[5,4-c] pyridine-5(4H)-carboxamide | 389 | 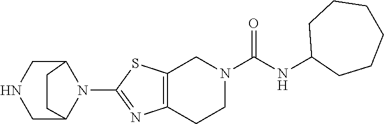 |
| Example 54 | 2-(3,8-diazabicyclo[3.2.1]octan-8-yl)- N-(cyclohexylmethyl)-6,7-dihydrothiazolo[5,4-c] pyridine-5(4H)-carboxamide | 389 | 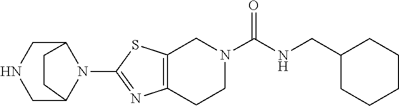 [SMILES: O=C(NCC1CCCCC1)N1CCc2nc(N3C4CCC3CNC4)sc2C1] |
| Example 55 | 2-(3,8-diazabicyclo[3.2.1]octan-8-yl)- N-(4-fluorobenzyl)-6,7-dihydrothiazolo[5,4-c] pyridine-5(4H)-carboxamide | 401 | 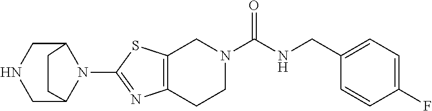 [SMILES: O=C(NCc1ccc(F)cc1)N1CCc2nc(N3C4CCC3CNC4)sc2C1] |
| Example 56 | 2-(3,8-diazabicyclo[3.2.1]octan-8- yl)-N-(4-(trifluoromethyl)benzyl)- 6,7-dihydrothiazolo[5,4-c] pyridine-5(4H)-carboxamide | 451 | 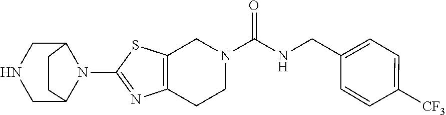 [SMILES: O=C(NCc1ccc(C(F)(F)F)cc1)N1CCc2nc(N3C4CCC3CNC4)sc2C1] |
| Example 57 | 2-(3,8-diazabicyclo[3.2.1]octan-8- yl)-N-phenyl-6,7-dihydrothiazolo[5,4-c] pyridine-5(4H)-carboxamide | 369 | 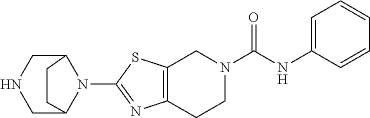 |
| Example 58 | 2-(3,8-diazabicyclo[3.2.1]octan-8-yl)- N-(4,4-difluorocyclohexyl)- 6,7-dihydrothiazolo[5,4-c] pyridine-5(4H)-carboxamide | 411 | 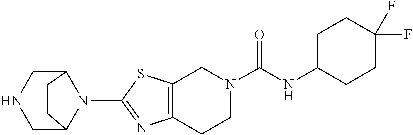 [SMILES: O=C(NC1CCC(F)(F)CC1)N1CCc2nc(N3C4CCC3CNC4)sc2C1] |
| Example 60 | (2-(3,8-diazabicyclo[3.2.1]octan-8- yl)-6,7-dihydrothiazolo[5,4-c]pyridin- 5(4H)-yl)(pyrrolidin-1-yl)methanone | 347 | 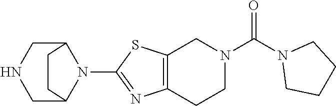 [SMILES: O=C(N1CCCC1)N1CCc2nc(N3C4CCC3CNC4)sc2C1] |
| Example 62 | 2-(3,8-diazabicyclo[3.2.1]octan-8-yl)- N-cyclopentyl-N-methyl- 6,7-dihydrothiazolo[5,4-c] pyridine-5(4H)-carboxamide | 375 | 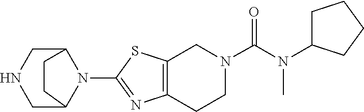 [SMILES: CN(C(=O)N1CCc2nc(N3C4CCC3CNC4)sc2C1)C1CCCC1] |
| Example 65 | 1-(2-(3,8-diazabicyclo[3.2.1]octan-8- yl)-6,7-dihydrothiazolo[5,4-c]pyridin- 5(4H)-yl)-2,2-dimethylpropan-1-one | 334 | 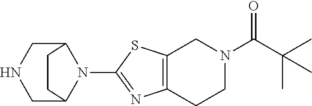 [SMILES: CC(C)(C)C(=O)N1CCc2nc(N3C4CCC3CNC4)sc2C1] |
| Example 66 | 1-(2-(3,8-diazabicyclo[3.2.1]octan-8- yl)-4,6-dihydro-5H-pyrrolo[3,4- d]thiazol-5-yl)-2-cyclopentylethan-1-one | 346 | 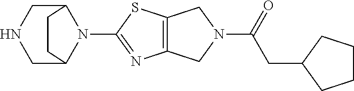 [SMILES: O=C(CC1CCCC1)N1Cc2nc(N3C4CCC3CNC4)sc2C1] |
| Example 67 | 1-(2-(3,8-diazabicyclo[3.2.1]octan-8- yl)-4,6-dihydro-5H-pyrrolo[3,4- d]thiazol-5-yl)-2-(4,4-difluorocyclohexyl) ethan-1-one | 396 | 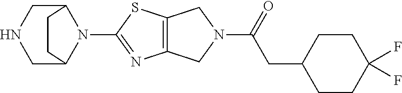 [SMILES: O=C(CC1CCC(F)(F)CC1)N1Cc2nc(N3C4CCC3CNC4)sc2C1] |
| Example 68 | 2-(3,8-diazabicyclo[3.2.1]octan-8-yl)- N-cyclopentyl-4,6-dihydro-5H- pyrrolo[3,4-d]thiazole-5-carboxamide | 347 | 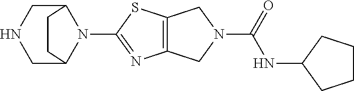 [SMILES: O=C(NC1CCCC1)N1Cc2nc(N3C4CCC3CNC4)sc2C1] |
| Example 69 | 2-(3,8-diazabicyclo[3.2.1]octan-8-yl)- N-(4,4-difluorocyclohexyl)-4,6- dihydro-5H-pyrrolo[3,4-d]thiazole-5- carboxamide | 397 | 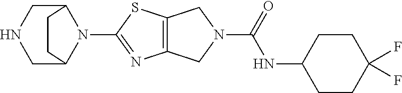 [SMILES: O=C(NC1CCC(F)(F)CC1)N1Cc2nc(N3C4CCC3CNC4)sc2C1] |
| Example 70 | 2-(3,8-diazabicyclo[3.2.1]octan-8- yl)-N-(4,4-difluorocyclohexyl)-6,7- dihydropyrazolo[1,5-a]pyrazine- 5(4H)-carboxamide | 394 | 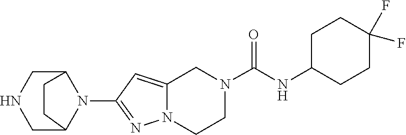 [SMILES: O=C(NC1CCC(F)(F)CC1)N1CCn2nc(N3C4CCC3CNC4)cc2C1] |
| Example 71 | 1-(2-(3,8-diazabicyclo[3.2.1]octan-8- 8-yl)-6,7-dihydropyrazolo[1,5- a]pyrazin-5(4H)-yl)-2-(4,4- difluorocyclohexyl)ethan-1-one | 393 | 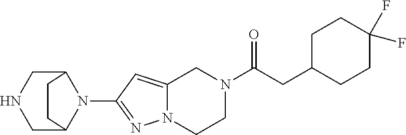 [SMILES: O=C(CC1CCC(F)(F)CC1)N1CCn2nc(N3C4CCC3CNC4)cc2C1] |
| Example 72 | 1-(2-(3,8-diazabicyclo[3.2.1]octan-8- yl)-6,7-dihydropyrazolo[1,5-a]pyrazin-5(4H)- yl)-2-(4-fluorophenoxy)ethan-1-one | 385 | 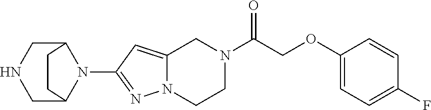 [SMILES: O=C(COc1ccc(F)cc1)N1CCn2nc(N3C4CCC3CNC4)cc2C1] |
| Example 73 | 1-(2-(3,8-diazabicyclo[3.2.1]octan-8- yl)-6,7-dihydrothiazolo[5,4-c]pyridin-5(4H)- yl)-2-((2-fluoropyridin-4-yl)oxy)ethan-1-one | 403 | 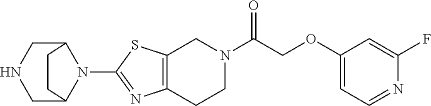 [SMILES: O=C(COc1ccnc(F)c1)N1CCc2nc(N3C4CCC3CNC4)sc2C1] |
| Example 74 | 1-(2-(3,8-diazabicyclo[3.2.1]octan-8- yl)-6,7-dihydrothiazolo[5,4-c]pyridin-5(4H)- yl)-2-((6-fluoropyridin-3-yl)oxy)ethan- 1-one | 403 | 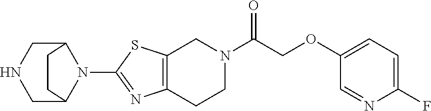 [SMILES: O=C(COc1ccc(F)nc1)N1CCc2nc(N3C4CCC3CNC4)sc2C1] |
| Example 75 | 2-cyclopentyl-1-(2-(3-methyl-3,8- diazabicyclo[3.2.1]octan-8-yl)- 6,7-dihydrothiazolo[5,4-c]pyridin-5(4H)- yl)ethan-1-one | 374 | 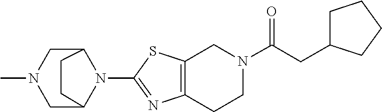 [SMILES: CN1CC2CCC(C1)N2c1nc2c(s1)CN(C(=O)CC1CCCC1)CC2] |
| Example 76 | 2-(4,4-difluorocyclohexyl)-1-(2-(3- methyl-3,8-diazabicyclo[3.2.1]octan-8-yl)- 6,7-dihydrothiazolo[5,4-c]pyridin-5(4H)- yl)ethan-1-one | 424 | 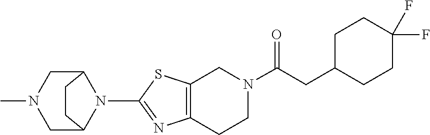 [SMILES: CN1CC2CCC(C1)N2c1nc2c(s1)CN(C(=O)CC1CCC(F)(F)CC1)CC2] |
| Example 77 | N-(4,4-difluorocyclohexyl)-2-(3- methyl-3,8-diazabicyclo[3.2.1]octan-8-yl)- 6,7-dihydrothiazolo[5,4-c]pyridine-5(4H)- carboxamide | 425 | 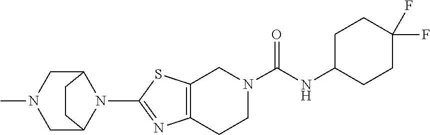 [SMILES: CN1CC2CCC(C1)N2c1nc2c(s1)CN(C(=O)NC1CCC(F)(F)CC1)CC2] |
| Example 78 | N-cyclopentyl-2-(3-methyl-3,8- diazabicyclo[3.2.1]octan-8-yl)- 6,7-dihydrothiazolo[5,4-c]pyridine-5(4H)- carboxamide | 375 | 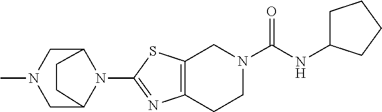 [SMILES: CN1CC2CCC(C1)N2c1nc2c(s1)CN(C(=O)NC1CCCC1)CC2] |
| Example 79 | 2-((2-fluoropyridin-4-yl)oxy)-1-(2-(3- methyl-3,8-diazabicyclo[3.2.1]octan- 8-yl)-6,7-dihydrothiazolo[5,4-c]pyridin- 5(4H)-yl)ethan-1-one | 417 | 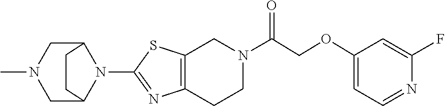 [SMILES: CN1CC2CCC(C1)N2c1nc2c(s1)CN(C(=O)COc1ccnc(F)c1)CC2] |
| Example 80 | 2-((6-fluoropyridin-3-yl)oxy)-1-(2-(3- methyl-3,8-diazabicyclo[3.2.1]octan-8-yl)- 6,7-dihydrothiazolo[5,4-c]pyridin-5(4H)- yl)ethan-1-one | 417 | 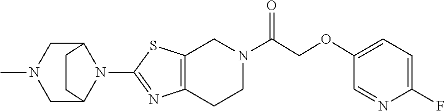 [SMILES: CN1CC2CCC(C1)N2c1nc2c(s1)CN(C(=O)COc1ccc(F)nc1)CC2] |
| Example 81 | N-(4,4-difluorocyclohexyl)-2-(3- methyl-3,8-diazabicyclo[3.2.1]octan-8-yl)- 4,6-dihydro-5H-pyrrolo[3,4-d]thiazole-5- carboxamide | 411 | 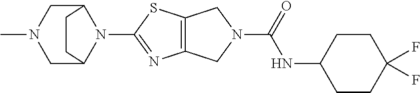 [SMILES: CN1CC2CCC(C1)N2c1nc2c(s1)CN(C(=O)NC1CCC(F)(F)CC1)C2] |
| Example 82 | N-cyclopentyl-2-(3-methyl-3,8- diazabicyclo[3.2.1]octan-8-yl)-4,6- dihydro-5H-pyrrolo[3,4-d]thiazole-5- carboxamide | 361 | 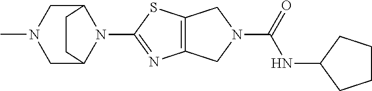 [SMILES: CN1CC2CCC(C1)N2c1nc2c(s1)CN(C(=O)NC1CCCC1)C2] |
| Example 83 | 2-cyclopentyl-1-(2-(3-methyl-3,8- diazabicyclo[3.2.1]octan-8-yl)-4,6- dihydro-5H-pyrrolo[3,4-d]thiazol-5- yl)ethan-1-one | 360 | 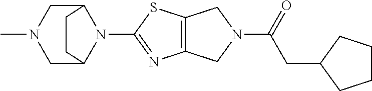 [SMILES: CN1CC2CCC(C1)N2c1nc2c(s1)CN(C(=O)CC1CCCC1)C2] |
| Example 84 | 2-(4,4-difluorocyclohexyl)-1-(2-(3- methyl-3,8-diazabicyclo[3.2.1]octan-8-yl)-4,6- dihydro-5H-pyrrolo[3,4-d]thiazol-5- yl)ethan-1-one | 410 | 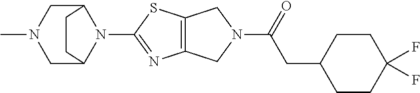 [SMILES: CN1CC2CCC(C1)N2c1nc2c(s1)CN(C(=O)CC1CCC(F)(F)CC1)C2] |
| Example 85 | N-(4,4-difluorocyclohexyl)-2-(3- methyl-3,8-diazabicyclo[3.2.1]octan-8-yl)-6,7- dihydropyrazolo[1,5-a]pyrazine- 5(4H)-carboxamide | 408 | 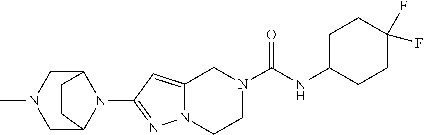 [SMILES: CN1CC2CCC(C1)N2c1cc2n(n1)CCN(C(=O)NC1CCC(F)(F)CC1)C2] |
| Example 86 | 2-(4,4-difluorocyclohexyl)-1-(2-(3- methyl-3,8-diazabicyclo[3.2.1]octan- 8-yl)-6,7-dihydropyrazolo[1,5- a]pyrazin-5(4H)-yl)ethan-1-one | 407 | 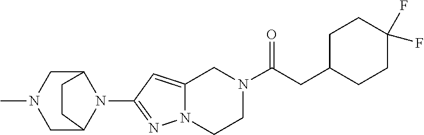 [SMILES: CN1CC2CCC(C1)N2c1cc2n(n1)CCN(C(=O)CC1CCC(F)(F)CC1)C2] |
| Example 97 | 1-(2-(3,8-diazabicyclo[3.2.1]octan-8- yl)-5,6-dihydro-[1,2,4]triazolo[1,5- a]pyrazin-7(8H)-yl)-2-(4,4- difluorocyclohexyl)ethan-1-one | 394 | 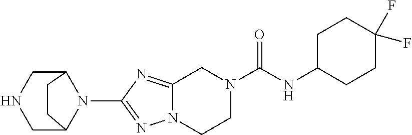 [SMILES: O=C(NC1CCC(F)(F)CC1)N1CCn2nc(N3C4CCC3CNC4)nc2C1] |
| Example 98 | 1-(2-(3,8-diazabicyclo[3.2.1]octan-8- yl)-7,7-dimethyl-6,7-dihydrothiazolo[5,4-c] pyridin-5(4H)-yl)-2-(4,4-difluorocyclohexyl) ethan-1-one | 438 | 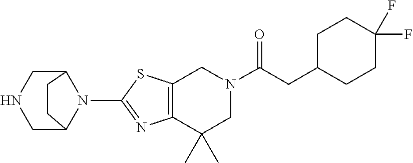 [SMILES: CC1(C)CN(C(=O)CC2CCC(F)(F)CC2)Cc2sc(N3C4CCC3CNC4)nc21] |
| Example 99 | 1-(2-(3,8-diazabicyclo[3.2.1]octan-8- yl)-7,7-dimethyl-6,7-dihydrothiazolo[5,4-c] pyridin-5(4H)-yl)-2-cyclopentylethan-1-one | 388 | 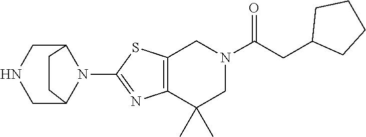 [SMILES: CC1(C)CN(C(=O)CC2CCCC2)Cc2sc(N3C4CCC3CNC4)nc21] |
| Example 100 | 2-(4,4-difluorocyclohexyl)-1-(7,7- dimethyl-2-(piperazin-1-yl)-6,7- dihydrothiazolo[5,4-c]pyridin-5(4H)- yl)ethan-1-one | 412 | 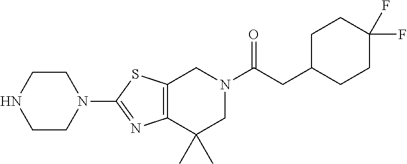 [SMILES: CC1(C)CN(C(=O)CC2CCC(F)(F)CC2)Cc2sc(N3CCNCC3)nc21] |
| Example 101 | 2-cyclopentyl-1-(7,7-dimethyl-2- (piperazin-1-yl)-6,7-dihydrothiazolo[5,4-c] pyridin-5(4H)-yl)ethan-1-one | 362 | 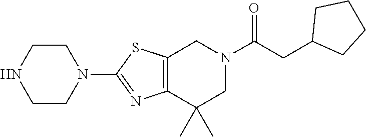 [SMILES: CC1(C)CN(C(=O)CC2CCCC2)Cc2sc(N3CCNCC3)nc21] |
| Example 102 | 2-cyclopentyl-1-(7,7-dimethyl-2-(3- methyl-3,8-diazabicyclo[3.2.1]octan-8-yl)- 6,7-dihydrothiazolo[5,4-c]pyridin-5(4H)- yl)ethan-1-one | 402 | 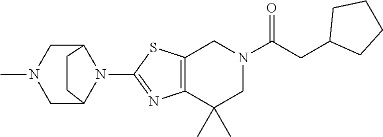 [SMILES: CN1CC2CCC(C1)N2c1nc2c(s1)CN(C(=O)CC1CCCC1)CC2(C)C] |
| Example 103 | 2-(4,4-difluorocyclohexyl)-1-(7,7- dimethyl-2-(3-methyl-3,8- diazabicyclo[3.2.1]octan-8-yl)- 6,7-dihydrothiazolo[5,4-c]pyridin-5(4H)- yl)ethan-1-one | 452 | 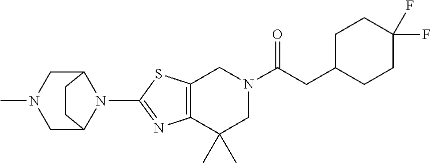 [SMILES: CN1CC2CCC(C1)N2c1nc2c(s1)CN(C(=O)CC1CCC(F)(F)CC1)CC2(C)C] |
| Example 104 | 2-(4,4-difluorocyclohexyl)-1-(7,7- dimethyl-2-(4-methylpiperazin-1-yl)- 6,7-dihydrothiazolo[5,4-c]pyridin- 5(4H)-yl)ethan-1-one | 426 | 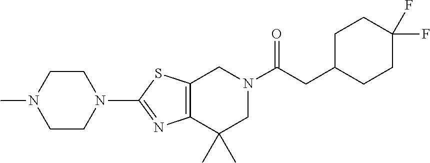 [SMILES: CN1CCN(c2nc3c(s2)CN(C(=O)CC2CCC(F)(F)CC2)CC3(C)C)CC1] |
| Example 107 | 2-(3,8-diazabicyclo[3.2.1]octan-8-yl)- 5-((cyclopentylmethyl)sulfonyl)- 4,5,6,7-tetrahydrothiazolo[5,4-c]pyridine | 396 | 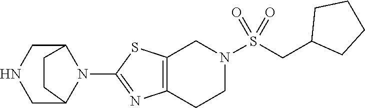 [SMILES: O=S(=O)(CC1CCCC1)N1CCc2nc(N3C4CCC3CNC4)sc2C1] |
| Example 108 | 2-(3,8-diazabicyclo[3.2.1]octan-8-yl)- N-cyclopentyl-6,7-dihydrothiazolo[5,4-c] pyridine-5(4H)-sulfonamide | 397 | 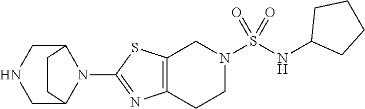 [SMILES: O=S(=O)(NC1CCCC1)N1CCc2nc(N3C4CCC3CNC4)sc2C1] |
| Example 109 | 2-cyclopentyl-1-(2-(piperazin-1-yl)- 6,7-dihydrothiazolo[5,4-c]pyridin-5(4H)- yl)ethan-1-one | 334 | 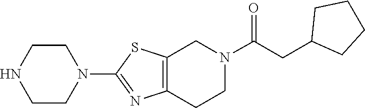 [SMILES: O=C(CC1CCCC1)N1CCc2nc(N3CCNCC3)sc2C1] |
| Example 110 | 2-(4-fluorophenoxy)-1-(2-(piperazin- 1-yl)-6,7-dihydrothiazolo[5,4-c]pyridin- 5(4H)-yl)ethan-1-one | 376 | 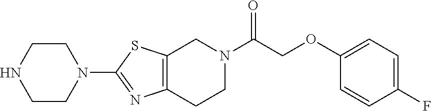 [SMILES: O=C(COc1ccc(F)cc1)N1CCc2nc(N3CCNCC3)sc2C1] |
| Example 116 | (S)-2-cyclopentyl-1-(2-(3- (hydroxymethyl)piperazin-1-yl)- 6,7-dihydrothiazolo[5,4-c]pyridin-5(4H)- yl)ethan-1-one | 364 | 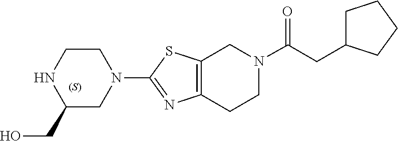 |
| Example 117 | (R)-2-cyclopentyl-1-(2-(3- (hydroxymethyl)piperazin-1-yl)- 6,7-dihydrothiazolo[5,4-c]pyridin-5(4H)- yl)ethan-1-one | 364 | 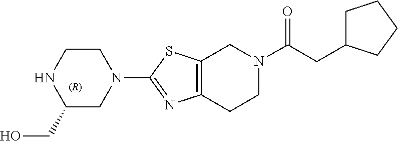 |
| Example 118 | 2-cyclopentyl-1-(2-(piperidin-4-yl)- 6,7-dihydrothiazolo[5,4-c]pyridin-5(4H)- yl)ethan-1-one | 333 | 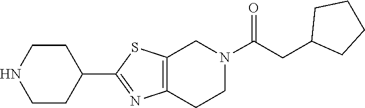 [SMILES: O=C(CC1CCCC1)N1CCc2nc(C3CCNCC3)sc2C1] |
| Example 119 | 1-(2-(3,8-diazabicyclo[3.2.1]octan-8- yl)-6,7-dihydrothiazolo[5,4-c]pyridin-5(4H)-yl)- 2-(3,3-dimethylcyclobutyl)ethan-1-one | 374 | 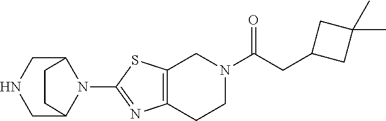 [SMILES: CC1(C)CC(CC(=O)N2CCc3nc(N4C5CCC4CNC5)sc3C2)C1] |
| Example 120 | (2-(3,8-diazabicyclo[3.2.1]octan-8- yl)-6,7-dihydrothiazolo[5,4-c]pyridin- 5(4H)-yl)(2,2-dimethylpyrrolidin-1- yl)methanone | 375 | 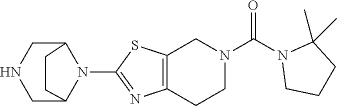 [SMILES: CC1(C)CCCN1C(=O)N1CCc2nc(N3C4CCC3CNC4)sc2C1] |
| Example 121 | (2-(3,8-diazabicyclo[3.2.1]octan-8- yl)-6,7-dihydrothiazolo[5,4-c]pyridin- 5(4H)-yl)(3,3-dimethylpyrrolidin-1- yl)methanone | 375 | 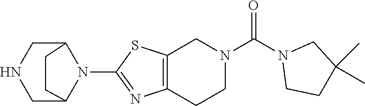 [SMILES: CC1(C)CCN(C(=O)N2CCc3nc(N4C5CCC4CNC5)sc3C2)C1] |
| Example 122 | (2-(3,8-diazabicyclo[3.2.1]octan-8- yl)-6,7-dihydrothiazolo[5,4-c]pyridin- 5(4H)-yl)(2-azaspiro[4.4]nonan-2- yl)methanone | 401 | 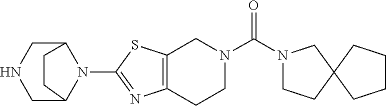 [SMILES: O=C(N1CCc2nc(N3C4CCC3CNC4)sc2C1)N1CCC2(CCCC2)C1] |
| Example 123 | (2-(3,8-diazabicyclo[3.2.1]octan-8- yl)-6,7-dihydrothiazolo[5,4-c]pyridin- 5(4H)-yl)(6-azaspiro[3.4]octan-6- yl)methanone | 387 | 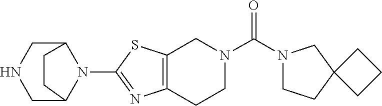 [SMILES: O=C(N1CCc2nc(N3C4CCC3CNC4)sc2C1)N1CCC2(CCC2)C1] |
| Example 124 | 2-(3,8-diazabicyclo[3.2.1]octan-8-yl)- N-(1-methylcyclobutyl)- 6,7-dihydrothiazolo[5,4-c]pyridine-5(4H)- carboxamide | 361 | 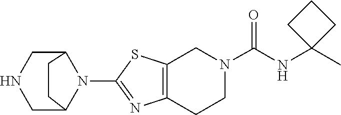 [SMILES: CC1(NC(=O)N2CCc3nc(N4C5CCC4CNC5)sc3C2)CCC1] |
| Example 125 | 2-(3,8-diazabicyclo[3.2.1]octan-8-yl)- N-(1-methylcyclopentyl)- 6,7-dihydrothiazolo[5,4-c]pyridine-5(4H)- carboxamide | 375 | 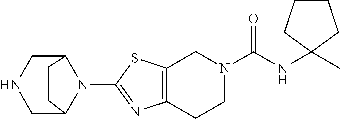 [SMILES: CC1(NC(=O)N2CCc3nc(N4C5CCC4CNC5)sc3C2)CCCC1] |
| Example 126 | 1-(2-(3,8-diazabicyclo[3.2.1]octan-8-yl)- 6,7-dihydropyrazolo[1,5-a]pyrazin-5(4H)-yl)- 2,2-difluoro-2-phenylethan-1-one | 387 | 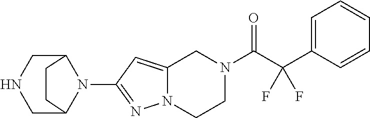 [SMILES: O=C(N1CCn2nc(N3C4CCC3CNC4)cc2C1)C(F)(F)c1ccccc1] |
| Example 127 | 1-(2-(3,8-diazabicyclo[3.2.1]octan-8- yl)-dihydropyrazolo[1,5-a]pyrazin-5(4H)- yl)-2-cyclopentylethan-1-one | 343 | 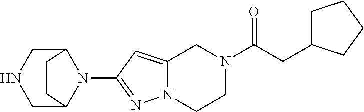 [SMILES: O=C(CC1CCCC1)N1CCn2nc(N3C4CCC3CNC4)cc2C1] |
| Example 128 | 1-(2-(3,8-diazabicyclo[3.2.1]octan-8- yl)-6,7-dihydropyrazolo[1,5-a]pyrazin-5(4H)- yl)-2-(1-methylcyclopentyl)ethan-1-one | 357 | 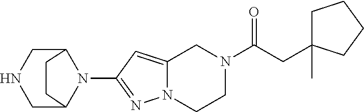 [SMILES: CC1(CC(=O)N2CCn3nc(N4C5CCC4CNC5)cc3C2)CCCC1] |
| Example 129 | 1-(2-(3,8-diazabicyclo[3.2.1]octan-8- yl)-6,7-dihydrothiazolo[5,4-c]pyridin- 5(4H)-yl)-2-(2-(2-methoxyethoxy)phenoxy) ethan-1-one | 458 | 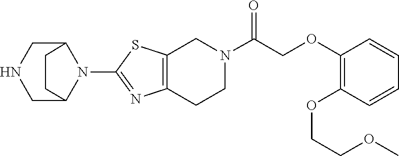 [SMILES: COCCOc1ccccc1OCC(=O)N1CCc2nc(N3C4CCC3CNC4)sc2C1] |
| Example 130 | 1-(2-(3,8-diazabicyclo[3.2.1]octan-8-yl)- 6,7-dihydrothiazolo[5,4-c]pyridin-5(4H)-yl)-2- (1-methylcyclobutyl)ethan-1-one | 360 | 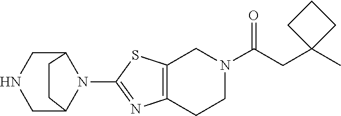 [SMILES: CC1(CC(=O)N2CCc3nc(N4C5CCC4CNC5)sc3C2)CCC1] |
| Example 131 | 1-(2-(3,8-diazabicyclo[3.2.1]octan-8- 6,7-dihydrothiazolo[5,4-c]pyridin-5(4H)- yl)-2-(4-fluorophenyl)-2-methoxyethan-1- one | 416 | 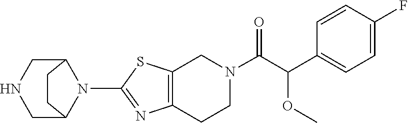 [SMILES: COC(C(=O)N1CCc2nc(N3C4CCC3CNC4)sc2C1)c1ccc(F)cc1] |
| Example 132 | 1-(2-(3,8-diazabicyclo[3.2.1]octan-8-yl)- 6,7-dihydrothiazolo[5,4-c]pyridin-5(4H)-yl)-2- (4-fluorophenyl)-2-hydroxyethan-1-one | 402 | 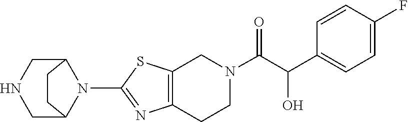 [SMILES: O=C(C(O)c1ccc(F)cc1)N1CCc2nc(N3C4CCC3CNC4)sc2C1] |
| Example 133 | (2-(3,8-diazabicyclo[3.2.1]octan-8-yl)- 6,7-dihydrothiazolo[5,4-c]pyridin-5(4H)-yl) (tetrahydrofuran-2-yl)methanone | 348 | 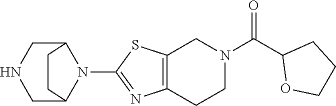 [SMILES: O=C(C1CCCO1)N1CCc2nc(N3C4CCC3CNC4)sc2C1] |
| Example 134 | 1-(2-(3,8-diazabicyclo[3.2.1]octan-8- yl)-6,7-dihydrothiazolo[5,4-c]pyridin-5(4H)- yl)-2-(1-methoxycyclopentyl)ethan-1-one | 390 | 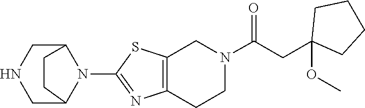 [SMILES: COC1(CC(=O)N2CCc3nc(N4C5CCC4CNC5)sc3C2)CCCC1] |
| Example 135 | 1-(2-(3,8-diazabicyclo[3.2.1]octan-8- yl)-6,7-dihydrothiazolo[5,4-c]pyridin-5(4H)- yl)-2-(1-(methoxymethyl)cyclopentyl)ethan-1- one | 404 | 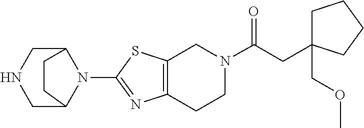 [SMILES: COCC1(CC(=O)N2CCc3nc(N4C5CCC4CNC5)sc3C2)CCCC1] |
| Example 136 | 1-(2-(3,8-diazabicyclo[3.2.1]octan-8- yl)-6,7-dihydrothiazolo[5,4-c]pyridin- 5(4H)-yl)-2-(pyrazin-2-yl)ethan-1-one | 370 | 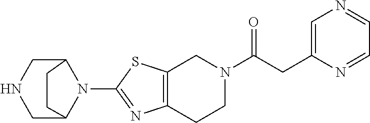 [SMILES: O=C(Cc1cnccn1)N1CCc2nc(N3C4CCC3CNC4)sc2C1] |
| Example 137 | 1-(2-(3,8-diazabicyclo[3.2.1]octan-8- yl)-6,7-dihydrothiazolo[5,4-c]pyridin-5(4H)- yl)-2-(3,3-difluorocyclopentyl)ethan-1-one | 396 | 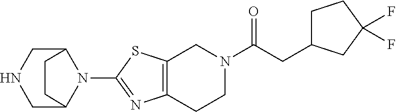 [SMILES: O=C(CC1CCC(F)(F)C1)N1CCc2nc(N3C4CCC3CNC4)sc2C1] |
| Example 138 | 1-(2-(3,8-diazabicyclo[3.2.1]octan-8- yl)-6,7-dihydrothiazolo[5,4-c]pyridin- 5(4H)-yl)-2-(2-methoxyphenoxy)ethan-1-one | 414 | 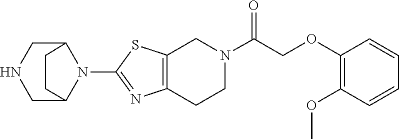 [SMILES: COc1ccccc1OCC(=O)N1CCc2nc(N3C4CCC3CNC4)sc2C1] |
| Example 139 | 1-(2-(3,8-diazabicyclo[3.2.1]octan-8- yl)-6,7-dihydrothiazolo[5,4-c]pyridin-5(4H)- yl)-2-(2-methoxyphenyl)ethan-1-one | 398 | 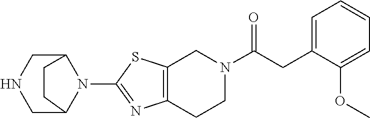 [SMILES: COc1ccccc1CC(=O)N1CCc2nc(N3C4CCC3CNC4)sc2C1] |
| Example 140 | 1-(2-(3,8-diazabicyclo[3.2.1]octan-8- yl)-6,7-dihydrothiazolo[5,4-c] pyridin-5(4H)-yl)-2-(4-fluoro-2- methoxyphenyl)ethan-1-one | 416 | 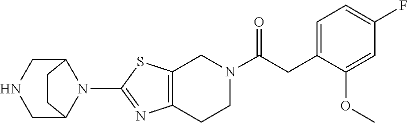 [SMILES: COc1cc(F)ccc1CC(=O)N1CCc2nc(N3C4CCC3CNC4)sc2C1] |
| Example 141 | 1-(2-(3,8-diazabicyclo[3.2.1]octan-8- yl)-6,7-dihydrothiazolo[5,4-c]pyridin- 5(4H)-yl)-2-(2-fluoropyridin-4-yl)ethan- 1-one | 387 | 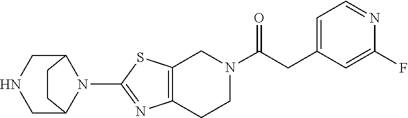 [SMILES: O=C(Cc1ccnc(F)c1)N1CCc2nc(N3C4CCC3CNC4)sc2C1] |
| Example 142 | 1-(2-(3,8-diazabicyclo[3.2.1]octan-8- yl)-6,7-dihydrothiazolo[5,4-c]pyridin- 5(4H)-yl)-3-(6-fluoropyridin-3- yl)propan-1-one | 401 | 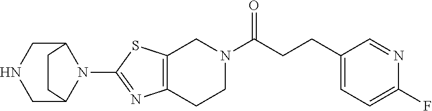 [SMILES: O=C(CCc1ccc(F)nc1)N1CCc2nc(N3C4CCC3CNC4)sc2C1] |
| Example 143 | 1-(2-(3,8-diazabicyclo[3.2.1]octan-8- yl)-6,7-dihydrothiazolo[5,4-c]pyridin- 5(4H)-yl)-2-(4-fluoro-2- methoxyphenoxy)ethan-1-one | 432 | 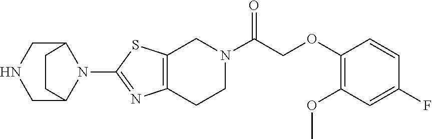 [SMILES: COc1cc(F)ccc1OCC(=O)N1CCc2nc(N3C4CCC3CNC4)sc2C1] |
| Example 144 | 1-(2-(3,8-diazabicyclo[3.2.1]octan-8- yl)-6,7-dihydrothiazolo[5,4-c]pyridin- 5(4H)-yl)-2-yl)-2-(2,4-difluorophenyl) ethan-1-one | 404 | 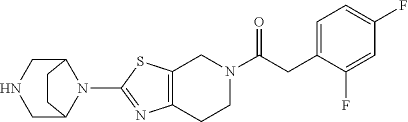 [SMILES: O=C(Cc1ccc(F)cc1F)N1CCc2nc(N3C4CCC3CNC4)sc2C1] |
| Example 145 | 1-(2-(3,8-diazabicyclo[3.2.1]octan-8- yl)-6,7-dihydrothiazolo[5,4-c]pyridin- 5(4H)-yl)-2-(4-fluoro-2- methoxyphenoxy)ethan-1-one | 416 | 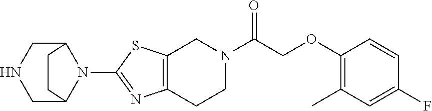 [SMILES: Cc1cc(F)ccc1OCC(=O)N1CCc2nc(N3C4CCC3CNC4)sc2C1] |
| Example 146 | 1-(2-(3,8-diazabicyclo[3.2.1]octan-8- yl)-6,7-dihydrothiazolo[5,4-c]pyridin- 5(4H)-yl)-2-cyclopentyl-2- methylpropan-1-one | 388 | 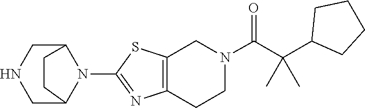 [SMILES: CC(C)(C(=O)N1CCc2nc(N3C4CCC3CNC4)sc2C1)C1CCCC1] |
| Example 147 | 1-(2-(3,8-diazabicyclo[3.2.1]octan-8- 8-yl)-6,7-dihydrothiazolo[5,4-c] pyridin-5(4H)-yl)-3-(4-fluoro-2- methoxyphenyl)propan-1-one | 430 | 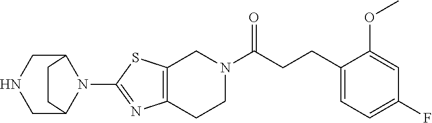 [SMILES: COc1cc(F)ccc1CCC(=O)N1CCc2nc(N3C4CCC3CNC4)sc2C1] |
| Example 148 | 1-(2-(3,8-diazabicyclo[3.2.1]octan-8- yl)-6,7-dihydrothiazolo[5,4-c]pyridin- 5(4H)-yl)-2-(2,4-difluorophenoxy) ethan-1-one | 420 | 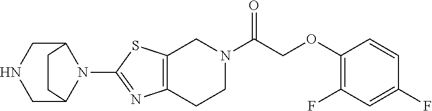 [SMILES: O=C(COc1ccc(F)cc1F)N1CCc2nc(N3C4CCC3CNC4)sc2C1] |
| Example 149 | 1-(2-(3,8-diazabicyclo[3.2.1]octan-8- yl)-6,7-dihydrothiazolo[5,4-c]pyridin- 5(4H)-yl)-2-(cyclopentyloxy)ethan-1- one | 376 | 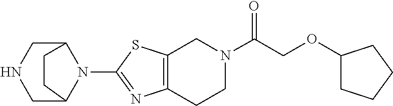 [SMILES: O=C(COC1CCCC1)N1CCc2nc(N3C4CCC3CNC4)sc2C1] |
| Example 150 | (2-(3,8-diazabicyclo[3.2.1]octan-8- yl)-6,7-dihydrothiazolo[5,4-c]pyridin- 5(4H)-yl)(1- methylcyclopentyl)methanone | 360 | 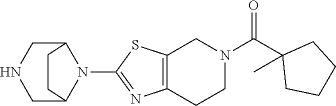 [SMILES: CC1(C(=O)N2CCc3nc(N4C5CCC4CNC5)sc3C2)CCCC1] |
| Example 151 | (2-(3,8-diazabicyclo[3.2.1]octan-8- yl)-6,7-dihydrothiazolo[5,4-c]pyridin- 5(4H)-yl)(1- fluorocyclopentyl)methanone | 364 | 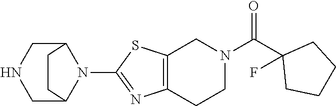 [SMILES: O=C(N1CCc2nc(N3C4CCC3CNC4)sc2C1)C1(F)CCCC1] |
| Example 152 | 3-(2-(3,8-diazabicyclo[3.2.1]octan-8- yl)-6,7-dihydrothiazolo[5,4-c]pyridin- 5(4H)-yl)-2-cyclopentyl-3- oxopropanenitrile | 385 | 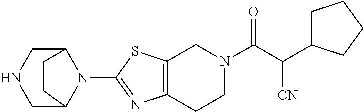 [SMILES: N#CC(C(=O)N1CCc2nc(N3C4CCC3CNC4)sc2C1)C1CCCC1] |
| Example 153 | 1-(2-(3,8-diazabicyclo[3.2.1]octan-8- yl)-6,7-dihydrothiazolo[5,4-c]pyridin- 5(4H)-yl)-2-(2,6-dimethylpyridin-4- yl)ethan-1-one | 397 | 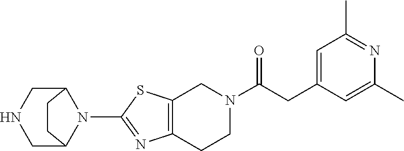 [SMILES: Cc1cc(CC(=O)N2CCc3nc(N4C5CCC4CNC5)sc3C2)cc(C)n1] |
| Example 154 | 1-(2-(3,8-diazabicyclo[3.2.1]octan-8- yl)-6,7-dihydrothiazolo[5,4-c]pyridin- 5(4H)-yl)-2,2-dimethylbutan-1-one | 348 | 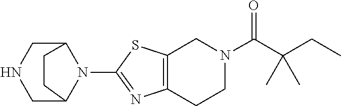 [SMILES: CCC(C)(C)C(=O)N1CCc2nc(N3C4CCC3CNC4)sc2C1] |
| Example 155 | 1-(2-(3,8-diazabicyclo[3.2.1]octan-8- yl)-6,7-dihydrothiazolo[5,4-c]pyridin- 5(4H)-yl)-2-(2-oxaspiro[3.5]nonan-7-yl) ethan-1-one | 416 | 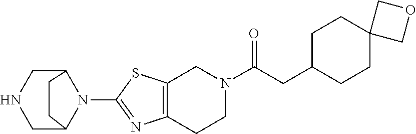 [SMILES: O=C(CC1CCC2(CC1)COC2)N1CCc2nc(N3C4CCC3CNC4)sc2C1] |
| Example 156 | 1-(2-(3,8-diazabicyclo[3.2.1]octan-8- yl)-6,7-dihydrothiazolo[5,4-c]pyridin- 5(4H)-yl)-2-(4-fluoro-2- methylphenyl)ethan-1-one | 400 | 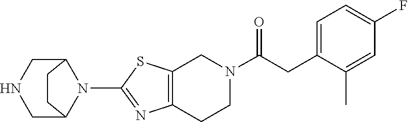 [SMILES: Cc1cc(F)ccc1CC(=O)N1CCc2nc(N3C4CCC3CNC4)sc2C1] |
| Example 157 | 1-(2-(3,8-diazabicyclo[3.2.1]octan-8- yl)-6,7-dihydrothiazolo[5,4-c]pyridin- 5(4H)-yl)-2-cyclopentyl-2- methoxyethan-1-one | 390 | 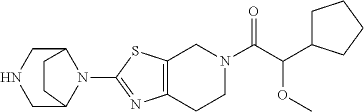 [SMILES: COC(C(=O)N1CCc2nc(N3C4CCC3CNC4)sc2C1)C1CCCC1] |
| Example 158 | 1-(2-(3,8-diazabicyclo[3.2.1]octan-8- yl)-6,7-dihydrothiazolo[5,4-c]pyridin- 5(4H)-yl)-2,3,3-trimethylbutan-1- one (enantiomer-1) | 362 | 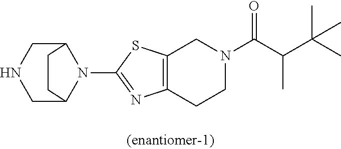 [SMILES: CC(C(=O)N1CCc2nc(N3C4CCC3CNC4)sc2C1)C(C)(C)C] |
| Example 159 | 1-(2-(3,8-diazabicyclo[3.2.1]octan-8- yl)-6,7-dihydrothiazolo[5,4-c]pyridin- 5(4H)-yl)-2,3,3-trimethylbutan-1-one (enantiomer-2) | 362 | 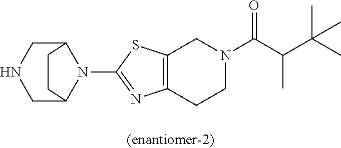 [SMILES: CC(C(=O)N1CCc2nc(N3C4CCC3CNC4)sc2C1)C(C)(C)C] |
| Example 160 | 1-(2-(3,8-diazabicyclo[3.2.1]octan-8- yl)-6,7-dihydrothiazolo[5,4-c]pyridin- 5(4H)-yl)-2-(bicyclo[2.2.1]heptan-1- yl)ethan-1-one | 386 | 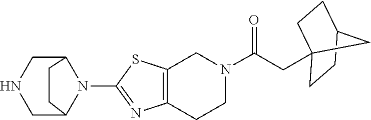 [SMILES: O=C(CC12CCC(CC1)C2)N1CCc2nc(N3C4CCC3CNC4)sc2C1] |
| Example 161 | 1-(2-(3,8-diazabicyclo[3.2.1]octan-8- yl)-6,7-dihydrothiazolo[5,4-c]pyridin- 5(4H)-yl)-2,2,3- trimethylbutan-1-one | 362 | 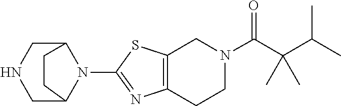 [SMILES: CC(C)C(C)(C)C(=O)N1CCc2nc(N3C4CCC3CNC4)sc2C1] |
| Example 162 | (2-(3,8-diazabicyclo[3.2.1]octan-8- yl)-6,7-dihydrothiazolo[5,4-c]pyridin- 5(4H)-yl)(spiro[2.4]heptan-1- yl)methanone | 372 | 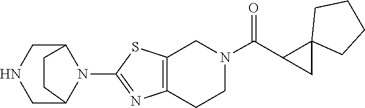 [SMILES: O=C(C1CC12CCCC2)N1CCc2nc(N3C4CCC3CNC4)sc2C1] |
| Example 163 | 1-(2-(3,8-diazabicyclo[3.2.1]octan-8- yl)-6,7-dihydrothiazolo[5,4-c]pyridin- 5(4H)-yl)-2-(2-isopropoxyphenoxy) ethan-1-one | 442 | 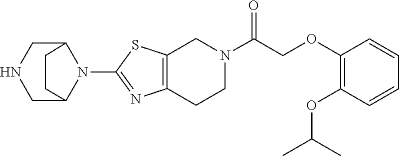 [SMILES: CC(C)Oc1ccccc1OCC(=O)N1CCc2nc(N3C4CCC3CNC4)sc2C1] |
| Example 164 | 1-(2-(3,8-diazabicyclo[3.2.1]octan-8- yl)-6,7-dihydrothiazolo[5,4-c]pyridin- 5(4H)-yl)-2-(2-(trifluoromethoxy) phenoxy)ethan-1-one | 468 | 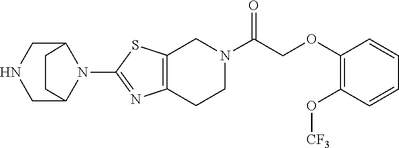 [SMILES: O=C(COc1ccccc1OC(F)(F)F)N1CCc2nc(N3C4CCC3CNC4)sc2C1] |
| Example 165 | 1-(2-(3,8-diazabicyclo[3.2.1]octan-8- yl)-6,7-dihydrothiazolo[5,4-c]pyridin- 5(4H)-yl)-2-(6-fluoropyridin-3- yl)ethan-1-one | 387 | 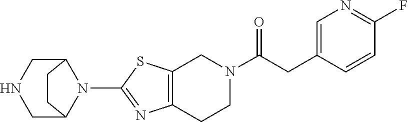 [SMILES: O=C(Cc1ccc(F)nc1)N1CCc2nc(N3C4CCC3CNC4)sc2C1] |
| Example 166 | 1-(2-(3,8-diazabicyclo[3.2.1]octan-8- yl)-6,7-dihydrothiazolo[5,4-c]pyridin- 5(4H)-yl)-2,2-difluoro-6-(6- fluoropyridin-3-yl)ethan-1-one | 423 | 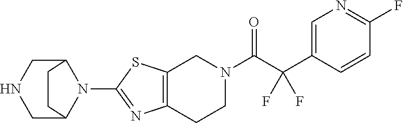 [SMILES: O=C(N1CCc2nc(N3C4CCC3CNC4)sc2C1)C(F)(F)c1ccc(F)nc1] |
| Example 167 | 2-(2-(2-(3,8-diazabicyclo[3.2.1]octan-8- yl)-6,7-dihydrothiazolo[5,4-c]pyridin- 5(4H)-yl)-2-oxoethoxy)benzonitrile | 409 | 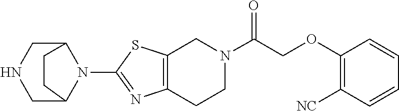 [SMILES: N#Cc1ccccc1OCC(=O)N1CCc2nc(N3C4CCC3CNC4)sc2C1] |
| Example 168 | 2-(2-(2-(3,8-diazabicyclo[3.2.1]octan-8- yl)-6,7-dihydrothiazolo[5,4-c]pyridin- 5(4H)-yl)-2-oxoethoxy)-3- methoxybenzonitrile | 439 | 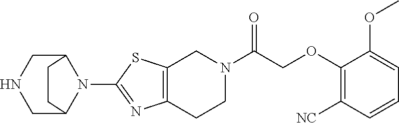 [SMILES: COc1cccc(C#N)c1OCC(=O)N1CCc2nc(N3C4CCC3CNC4)sc2C1] |
| Example 169 | 1-(2-(3,8-diazabicyclo[3.2.1]octan-8- 8-yl)-6,7-dihydrothiazolo[5,4-c]pyridin- 5(4H)-yl)-2-(cyclopentyloxy)propan- 1-one | 390 | 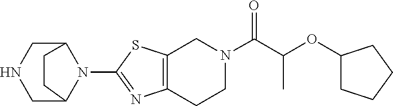 [SMILES: CC(OC1CCCC1)C(=O)N1CCc2nc(N3C4CCC3CNC4)sc2C1] |
| Example 170 | (2S)-1-(2-(3,8-diazabicyclo[3.2.1]octan-8- yl)-6,7-dihydrothiazolo[5,4-c]pyridin- 5(4H)-yl)-2-cyclopentyl-2- hydroxyethan-1-one | 376 | 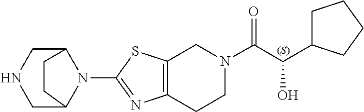 |
| Example 171 | (2R)-1-(2-(3,8-diazabicyclo[3.2.1]octan-8- yl)-6,7-dihydrothiazolo[5,4-c]pyridin- 5(4H)-yl)-2-cyclopentyl-2- hydroxyethan-1-one | 376 | 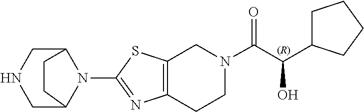 |
| Example 172 | 1-(2-(3,8-diazabicyclo[3.2.1]octan-8- yl)-6,7-dihydrothiazolo[5,4-c]pyridin- 5(4H)-yl)-2-(2,6- dimethoxyphenoxy)ethan-1-one | 444 | 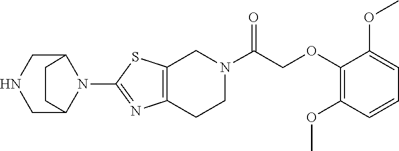 [SMILES: COc1cccc(OC)c1OCC(=O)N1CCc2nc(N3C4CCC3CNC4)sc2C1] |
| Example 173 | 1-(2-(3,8-diazabicyclo[3.2.1]octan-8- yl)-6,7-dihydrothiazolo[5,4-c]pyridin- 5(4H)-yl)-2-(2- (difluoromethyl)phenoxy)ethan-1-one | 434 | 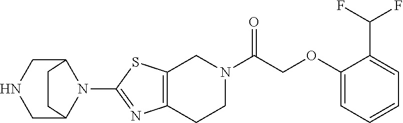 [SMILES: O=C(COc1ccccc1C(F)F)N1CCc2nc(N3C4CCC3CNC4)sc2C1] |
| Example 174 | 1-(2-(3,8-diazabicyclo[3.2.1]octan-8- yl)-6,7-dihydrothiazolo[5,4-c]pyridin- 5(4H)-yl)-3,3-dimethylpentan-1-one | 362 | 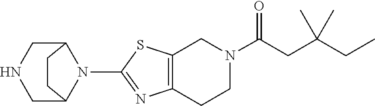 [SMILES: CCC(C)(C)CC(=O)N1CCc2nc(N3C4CCC3CNC4)sc2C1] |
| Example 175 | 1-(2-(3,8-diazabicyclo[3.2.1]octan-8- yl)-6,7-dihydrothiazolo[5,4-c]pyridin- 5(4H)-yl)-4,4-difluoro-3,3- dimethylbutan-1-one | 384 | 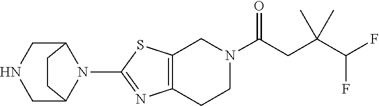 [SMILES: CC(C)(CC(=O)N1CCc2nc(N3C4CCC3CNC4)sc2C1)C(F)F] |
| Example 176 | (2R)-1-(2-(3,8-diazabicyclo[3.2.1]octan-8- yl)-6,7-dihydrothiazolo[5,4-c]pyridin- 5(4H)-yl)-2-hydroxy-3,3- dimethylbutan-1-one | 364 | 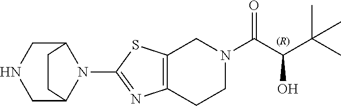 |
| Example 177 | (2R)-(2-(3,8-diazabicyclo[3.2.1]octan-8- yl)-6,7-dihydrothiazolo[5,4-c]pyridin- 5(4H)-yl)-2-cyclohexyl-2- hydroxyethan-1-one | 390 | 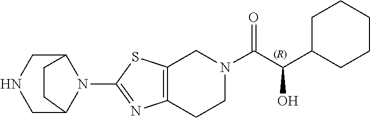 |
| Example 178 | 4-(2-(3,8-diazabicyclo[3.2.1]octan-8- yl)-6,7-dihydrothiazolo[5,4-c]pyridin- 5(4H)-yl)-2,2-dimethyl-4- oxobutanenitrile | 359 | 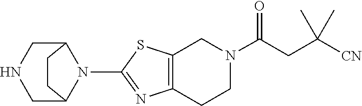 [SMILES: CC(C)(C#N)CC(=O)N1CCc2nc(N3C4CCC3CNC4)sc2C1] |
| Example 179 | (2-(3,8-diazabicyclo[3.2.1]octan-8- yl)-6,7-dihydrothiazolo[5,4-c]pyridin- 5(4H)-yl)(2,3-dihydrobenzofuran-2- yl)methanone | 396 | 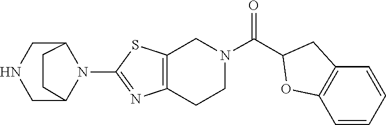 [SMILES: O=C(C1Cc2ccccc2O1)N1CCc2nc(N3C4CCC3CNC4)sc2C1] |
| Example 180 | (2-(3,8-diazabicyclo[3.2.1]octan-8- yl)-6,7-dihydrothiazolo[5,4-c]pyridin- 5(4H)-yl)(2,3-dihydrobenzo[b][1,4] dioxin-2-yl)methanone | 412 | 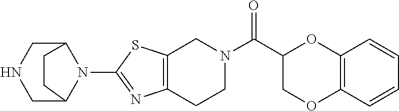 [SMILES: O=C(C1COc2ccccc2O1)N1CCc2nc(N3C4CCC3CNC4)sc2C1] |
| Example 181 | (2-(3,8-diazabicyclo[3.2.1]octan-8- yl)-6,7-dihydrothiazolo[5,4-c]pyridin- 5(4H)-yl)(chroman-2-yl)methanone | 410 | 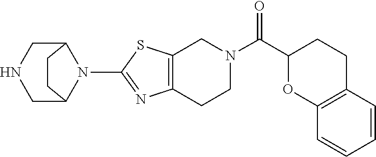 [SMILES: O=C(C1CCc2ccccc2O1)N1CCc2nc(N3C4CCC3CNC4)sc2C1] |
| Example 182 | (2-(3,8-diazabicyclo[3.2.1]octan-8- yl)-6,7-dihydrothiazolo[5,4-c]pyridin- 5(4H)-yl)(chroman-3-yl)methanone | 410 | 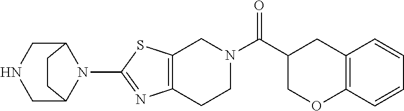 [SMILES: O=C(C1COc2ccccc2C1)N1CCc2nc(N3C4CCC3CNC4)sc2C1] |
| Example 183 | (2-(3,8-diazabicyclo[3.2.1]octan-8- yl)-6,7-dihydrothiazolo[5,4-c]pyridin- 5(4H)-yl)(isochroman-3-yl) methanone | 410 | 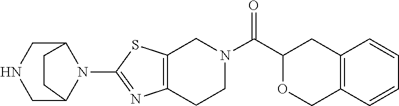 [SMILES: O=C(C1Cc2ccccc2CO1)N1CCc2nc(N3C4CCC3CNC4)sc2C1] |
| Example 184 | (2-(3,8-diazabicyclo[3.2.1]octan-8- yl)-6,7-dihydrothiazolo[5,4-c]pyridin- 5(4H)-yl)(2,3-dihydrobenzofuran-3- yl)methanone | 396 | 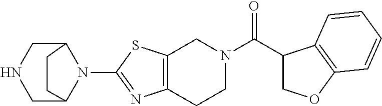 [SMILES: O=C(C1COc2ccccc21)N1CCc2nc(N3C4CCC3CNC4)sc2C1] |
| Example 185 | (2-(3,8-diazabicyclo[3.2.1]octan-8- yl)-6,7-dihydrothiazolo[5,4-c]pyridin- 5(4H)-yl)(benzofuran-2-yl) methanone | 394 | 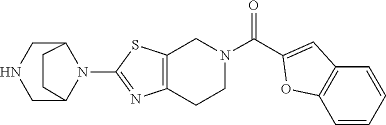 [SMILES: O=C(c1cc2ccccc2o1)N1CCc2nc(N3C4CCC3CNC4)sc2C1] |
| Example 186 | (2-(3,8-diazabicyclo[3.2.1]octan-8- yl)-6,7-dihydrothiazolo[5,4-c]pyridin- 5(4H)-yl)(isoquinolin-1- yl)methanone | 405 | 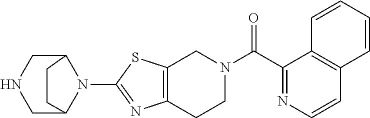 [SMILES: O=C(c1nccc2ccccc12)N1CCc2nc(N3C4CCC3CNC4)sc2C1] |
| Example 187 | (2-(3,8-diazabicyclo[3.2.1]octan-8- yl)-6,7-dihydrothiazolo[5,4-c]pyridin- 5(4H)-yl)(benzofuran-3- yl)methanone | 394 | 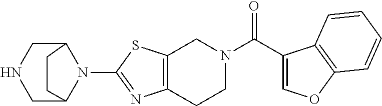 [SMILES: O=C(c1coc2ccccc12)N1CCc2nc(N3C4CCC3CNC4)sc2C1] |
| Example 188 | (2-(3,8-diazabicyclo[3.2.1]octan-8- yl)-6,7-dihydrothiazolo[5,4-c]pyridin- 5(4H)-yl)(1H-indol-2-yl) methanone | 393 | 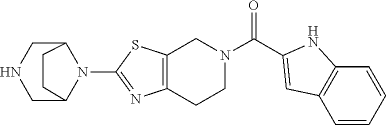 [SMILES: O=C(c1cc2ccccc2[nH]1)N1CCc2nc(N3C4CCC3CNC4)sc2C1] |
| Example 189 | (2-(3,8-diazabicyclo[3.2.1]octan-8- yl)-6,7-dihydrothiazolo[5,4-c]pyridin- 5(4H)-yl)(1-methyl-1H-indol-2- yl)methanone | 407 | 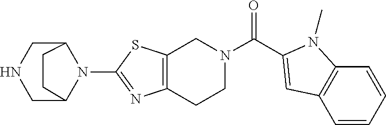 [SMILES: Cn1c(C(=O)N2CCc3nc(N4C5CCC4CNC5)sc3C2)cc2ccccc21] |
| Example 190 | (2-(3,8-diazabicyclo[3.2.1]octan-8- yl)-6,7-dihydrothiazolo[5,4-c]pyridin- 5(4H)-yl)benzo[d]oxazol-2- yl)methanone | 395 | 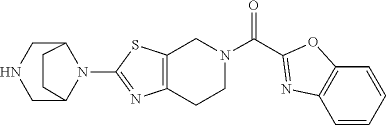 [SMILES: O=C(c1nc2ccccc2o1)N1CCc2nc(N3C4CCC3CNC4)sc2C1] |
| Example 192 | (2-(3,8-diazabicyclo[3.2.1]octan-8- yl)-6,7-dihydrothiazolo[5,4-c]pyridin- 5(4H)-yl)(isoquinolin-3-yl)methanone | 405 | 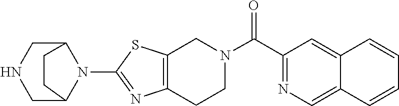 [SMILES: O=C(c1cc2ccccc2cn1)N1CCc2nc(N3C4CCC3CNC4)sc2C1] |
| Example 193 | (2-(3,8-diazabicyclo[3.2.1]octan-8- yl)-6,7-dihydrothiazolo[5,4-c]pyridin- 5(4H)-yl)(phenyl)methanone | 354 | 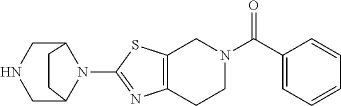 [SMILES: O=C(c1ccccc1)N1CCc2nc(N3C4CCC3CNC4)sc2C1] |
| Example 194 | (2-(3,8-diazabicyclo[3.2.1]octan-8- yl)-6,7-dihydrothiazolo[5,4-c]pyridin- 5(4H)-yl)(o-tolyl)methanone | 368 | 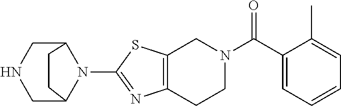 [SMILES: Cc1ccccc1C(=O)N1CCc2nc(N3C4CCC3CNC4)sc2C1] |
| Example 195 | (2-(3,8-diazabicyclo[3.2.1]octan-8- yl)-6,7-dihydrothiazolo[5,4-c]pyridin- 5(4H)-yl)(m-tolyl)methanone | 368 | 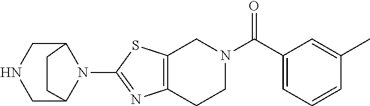 [SMILES: Cc1cccc(C(=O)N2CCc3nc(N4C5CCC4CNC5)sc3C2)c1] |
| Example 196 | (2-(3,8-diazabicyclo[3.2.1]octan-8- yl)-6,7-dihydrothiazolo[5,4-c]pyridin- 5(4H)-yl)(p-tolyl)methanone | 368 | 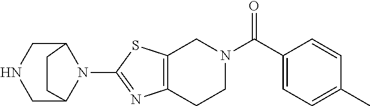 [SMILES: Cc1ccc(C(=O)N2CCc3nc(N4C5CCC4CNC5)sc3C2)cc1] |
| Example 197 | (2-(3,8-diazabicyclo[3.2.1]octan-8- yl)-6,7-dihydrothiazolo[5,4-c]pyridin- 5(4H)-yl)(2- methoxyphenyl)methanone | 384 | 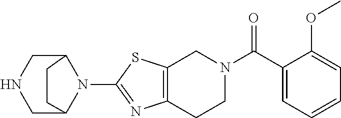 [SMILES: COc1ccccc1C(=O)N1CCc2nc(N3C4CCC3CNC4)sc2C1] |
| Example 198 | (2-(3,8-diazabicyclo[3.2.1]octan-8- yl)-6,7-dihydrothiazolo[5,4-c]pyridin- 5(4H)-yl)(4-fluorophenyl)methanone | 372 | 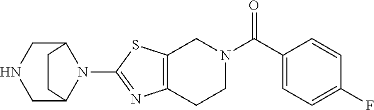 [SMILES: O=C(c1ccc(F)cc1)N1CCc2nc(N3C4CCC3CNC4)sc2C1] |
| Example 199 | (2-(3,8-diazabicyclo[3.2.1]octan-8- yl)-6,7-dihydrothiazolo[5,4-c]pyridin- 5(4H)-yl)(3- (difluoromethyl)phenyl)methanone | 404 | 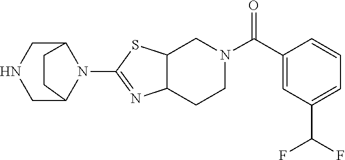 [SMILES: O=C(c1cccc(C(F)F)c1)N1CCC2N=C(N3C4CCC3CNC4)SC2C1] |
| Example 200 | (2-(3,8-diazabicyclo[3.2.1]octan-8- yl)-6,7-dihydrothiazolo[5,4-c]pyridin- 5(4H)-yl)(6-fluoropyridin-3- yl)methanone | 373 | 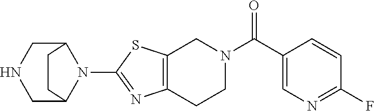 [SMILES: O=C(c1ccc(F)nc1)N1CCc2nc(N3C4CCC3CNC4)sc2C1] |
| Example 201 | (2-(3,8-diazabicyclo[3.2.1]octan-8- yl)-6,7-dihydrothiazolo[5,4-c]pyridin- 5(4H)-yl)(pyridin-2-yl)methanone | 355 | 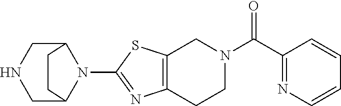 [SMILES: O=C(c1ccccn1)N1CCc2nc(N3C4CCC3CNC4)sc2C1] |
| Example 202 | (2-(3,8-diazabicyclo[3.2.1]octan-8- yl)-6,7-dihydrothiazolo[5,4-c]pyridin- 5(4H)-yl)(5-fluoropyridin-2- yl)methanone | 373 | 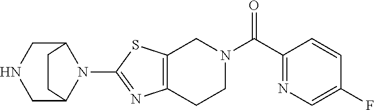 [SMILES: O=C(c1ccc(F)cn1)N1CCc2nc(N3C4CCC3CNC4)sc2C1] |
| Example 203 | (2-(3,8-diazabicyclo[3.2.1]octan-8- yl)-6,7-dihydrothiazolo[5,4-c]pyridin- 5(4H)-yl)(2- (difluoromethoxy)phenyl) methanone | 420 | 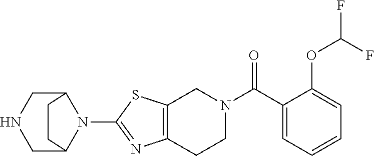 [SMILES: O=C(c1ccccc1OC(F)F)N1CCc2nc(N3C4CCC3CNC4)sc2C1] |
| Example 204 | (2-(3,8-diazabicyclo[3.2.1]octan-8- yl)-6,7-dihydrothiazolo[5,4-c]pyridin- 5(4H)-yl)(4- (difluoromethoxy)phenyl) methanone | 420 | 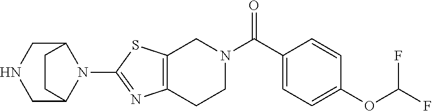 [SMILES: O=C(c1ccc(OC(F)F)cc1)N1CCc2nc(N3C4CCC3CNC4)sc2C1] |
| Example 205 | (2-(3,8-diazabicyclo[3.2.1]octan-8- yl)-6,7-dihydrothiazolo[5,4-c]pyridin- 5(4H)-yl)(3- (trifluoromethoxy)phenyl) methanone | 438 | 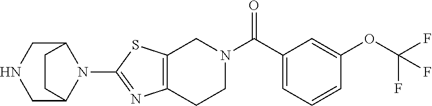 [SMILES: O=C(c1cccc(OC(F)(F)F)c1)N1CCc2nc(N3C4CCC3CNC4)sc2C1] |
| Example 206 | (2-(3,8-diazabicyclo[3.2.1]octan-8- yl)-6,7-dihydrothiazolo[5,4-c]pyridin- 5(4H)-yl)(3- (difluoromethoxy)phenyl) methanone | 420 | 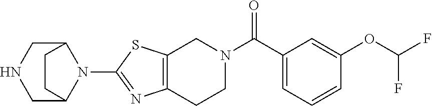 [SMILES: O=C(c1cccc(OC(F)F)c1)N1CCc2nc(N3C4CCC3CNC4)sc2C1] |
| Example 207 | (2-(3,8-diazabicyclo[3.2.1]octan-8- yl)-6,7-dihydrothiazolo[5,4-c]pyridin- 5(4H)-yl)(2- (trifluoromethoxy)phenyl) methanone | 438 | 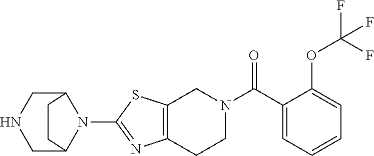 [SMILES: O=C(c1ccccc1OC(F)(F)F)N1CCc2nc(N3C4CCC3CNC4)sc2C1] |
| Example 208 | (2-(3,8-diazabicyclo[3.2.1]octan-8- yl)-6,7-dihydrothiazolo[5,4-c]pyridin- 5(4H)-yl)(4- (trifluoromethoxy)phenyl) methanone | 438 | 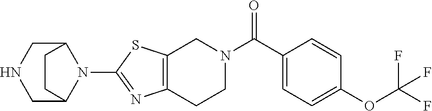 [SMILES: O=C(c1ccc(OC(F)(F)F)cc1)N1CCc2nc(N3C4CCC3CNC4)sc2C1] |
| Example 209 | (2-(3,8-diazabicyclo[3.2.1]octan-8- yl)-6,7-dihydrothiazolo[5,4-c]pyridin- 5(4H)-yl)(2-fluorophenyl) methanone | 372 | 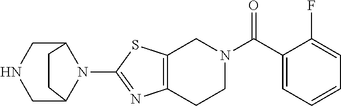 [SMILES: O=C(c1ccccc1F)N1CCc2nc(N3C4CCC3CNC4)sc2C1] |
| Example 210 | (2-(3,8-diazabicyclo[3.2.1]octan-8- yl)-6,7-dihydrothiazolo[5,4-c]pyridin- 5(4H)-yl)(2- chlorophenyl)methanone | 388 | 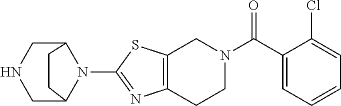 [SMILES: O=C(c1ccccc1Cl)N1CCc2nc(N3C4CCC3CNC4)sc2C1] |
| Example 211 | (2-(3,8-diazabicyclo[3.2.1]octan-8- yl)-6,7-dihydrothiazolo[5,4-c]pyridin- 5(4H)-yl)(2- isopropoxyphenyl)methanone | 412 | 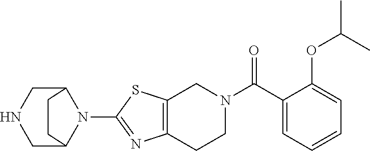 [SMILES: CC(C)Oc1ccccc1C(=O)N1CCc2nc(N3C4CCC3CNC4)sc2C1] |
| Example 212 | (2-(3,8-diazabicyclo[3.2.1]octan-8- yl)-6,7-dihydrothiazolo[5,4-c]pyridin- 5(4H)-yl)(2,4- difluorophenyl)methanone | 390 | 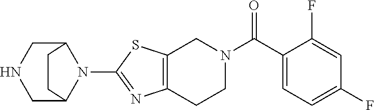 [SMILES: O=C(c1ccc(F)cc1F)N1CCc2nc(N3C4CCC3CNC4)sc2C1] |
| Example 213 | 2-(2-(3,8-diazabicyclo[3.2.1]octan-8- yl)-4,5,6,7-tetrahydrothiazolo[5,4- c]pyridine-5-carbonyl) benzonitrile | 379 | 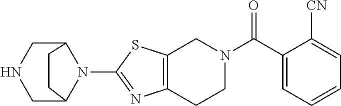 [SMILES: N#Cc1ccccc1C(=O)N1CCc2nc(N3C4CCC3CNC4)sc2C1] |
| Example 214 | (2-(3,8-diazabicyclo[3.2.1]octan-8- yl)-6,7-dihydrothiazolo[5,4-c]pyridin- 5(4H)-yl)(2-(methoxymethyl) phenyl)methanone | 398 | 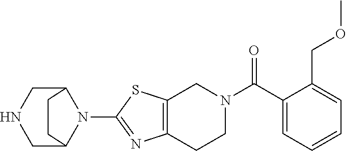 [SMILES: COCc1ccccc1C(=O)N1CCc2nc(N3C4CCC3CNC4)sc2C1] |
| Example 215 | (2-(3,8-diazabicyclo[3.2.1]octan-8- yl)-6,7-dihydrothiazolo[5,4-c]pyridin- 5(4H)-yl)(2-isopropylphenyl) methanone | 396 | 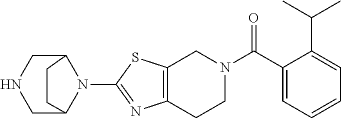 [SMILES: CC(C)c1ccccc1C(=O)N1CCc2nc(N3C4CCC3CNC4)sc2C1] |
| Example 216 | (2-(3,8-diazabicyclo[3.2.1]octan-8- yl)-6,7-dihydrothiazolo[5,4-c]pyridin- 5(4H)-yl)(4-fluoro-2- methylphenyl)methanone | 386 | 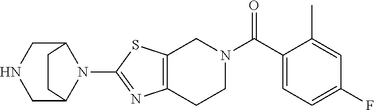 [SMILES: Cc1cc(F)ccc1C(=O)N1CCc2nc(N3C4CCC3CNC4)sc2C1] |
| Example 217 | (2-(3,8-diazabicyclo[3.2.1]octan-8- yl)-6,7-dihydrothiazolo[5,4-c]pyridin- 5(4H)-yl)(chroman-8-yl) methanone | 410 | 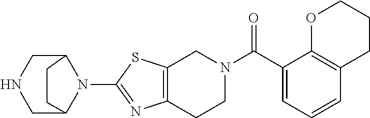 [SMILES: O=C(c1cccc2c1OCCC2)N1CCc2nc(N3C4CCC3CNC4)sc2C1] |
| Example 218 | (2-(3,8-diazabicyclo[3.2.1]octan-8- yl)-6,7-dihydrothiazolo[5,4-c]pyridin- 5(4H)-yl)(2-(difluoromethyl)phenyl) methanone | 404 | 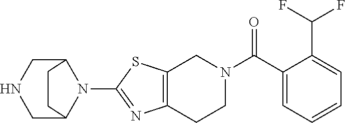 [SMILES: O=C(c1ccccc1C(F)F)N1CCc2nc(N3C4CCC3CNC4)sc2C1] |
| Example 219 | (2-(3,8-diazabicyclo[3.2.1]octan-8- yl)-6,7-dihydrothiazolo[5,4-c]pyridin- 5(4H)-yl)(2-methoxy-4- methylphenyl)methanone | 398 | 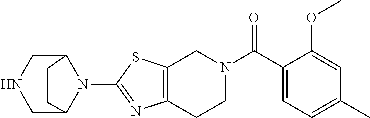 [SMILES: COc1cc(C)ccc1C(=O)N1CCc2nc(N3C4CCC3CNC4)sc2C1] |
| Example 220 | (2-(3,8-diazabicyclo[3.2.1]octan-8- yl)-6,7-dihydrothiazolo[5,4-c]pyridin- 5(4H)-yl)(2-cyclopropylphenyl) methanone | 394 | 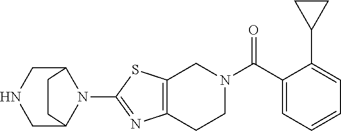 [SMILES: O=C(c1ccccc1C1CC1)N1CCc2nc(N3C4CCC3CNC4)sc2C1] |
| Example 221 | (2-(3,8-diazabicyclo[3.2.1]octan-8-yl)-6,7-dihydrothiazolo[5,4-c]pyridin- 5(4H)-yl)(6-chloropyridin-3- yl)methanone | 389 | 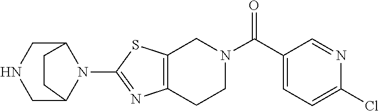 [SMILES: O=C(c1ccc(Cl)nc1)N1CCc2nc(N3C4CCC3CNC4)sc2C1] |
| Example 222 | (2-(3,8-diazabicyclo[3.2.1]octan-8- yl)-6,7-dihydrothiazolo[5,4-c]pyridin- 5(4H)-yl)(4-fluoro-2- methoxyphenyl)methanone | 402 | 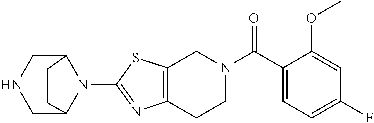 [SMILES: COc1cc(F)ccc1C(=O)N1CCc2nc(N3C4CCC3CNC4)sc2C1] |
| Example 223 | (2-(3,8-diazabicyclo[3.2.1]octan-8- yl)-6,7-dihydrothiazolo[5,4-c]pyridin- 5(4H)-yl)(2-(1,1- difluoroethyl)phenyl) methanone | 418 | 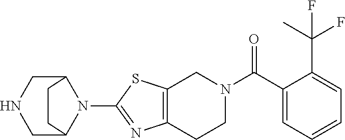 [SMILES: CC(F)(F)c1ccccc1C(=O)N1CCc2nc(N3C4CCC3CNC4)sc2C1] |
| Example 224 | (2-(3,8-diazabicyclo[3.2.1]octan-8- yl)-6,7-dihydrothiazolo[5,4-c]pyridin- 5(4H)-yl)(2-chloropyridin-3- yl)methanone | 389 | 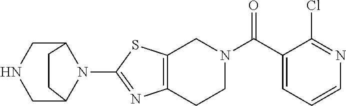 [SMILES: O=C(c1cccnc1Cl)N1CCc2nc(N3C4CCC3CNC4)sc2C1] |
| Example 225 | (2-(3,8-diazabicyclo[3.2.1]octan-8- yl)-6,7-dihydrothiazolo[5,4-c]pyridin- 5(4H)-yl)(2,4-dimethylphenyl) methanone | 382 | 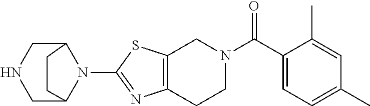 [SMILES: Cc1ccc(C(=O)N2CCc3nc(N4C5CCC4CNC5)sc3C2)c(C)c1] |
| Example 226 | (2-(3,8-diazabicyclo[3.2.1]octan-8- yl)-6,7-dihydrothiazolo[5,4-c]pyridin- 5(4H)-yl)(4-chlorophenyl) methanone | 388 | 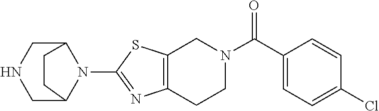 [SMILES: O=C(c1ccc(Cl)cc1)N1CCc2nc(N3C4CCC3CNC4)sc2C1] |
| Example 227 | (2-(3,8-diazabicyclo[3.2.1]octan-8- yl)-6,7-dihydrothiazolo[5,4-c]pyridin- 5(4H)-yl)(4-(trifluoromethyl) phenyl)methanone | 422 | 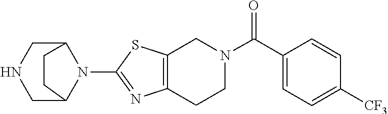 [SMILES: O=C(c1ccc(C(F)(F)F)cc1)N1CCc2nc(N3C4CCC3CNC4)sc2C1] |
| Example 228 | (2-(3,8-diazabicyclo[3.2.1]octan-8- yl)-6,7-dihydrothiazolo[5,4-c]pyridin- 5(4H)-yl)(4-cyclopropylphenyl) methanone | 394 | 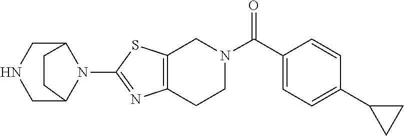 [SMILES: O=C(c1ccc(C2CC2)cc1)N1CCc2nc(N3C4CCC3CNC4)sc2C1] |
| Example 229 | (2-(3,8-diazabicyclo[3.2.1]octan-8- yl)-6,7-dihydrothiazolo[5,4-c]pyridin- 5(4H)-yl)(4-(difluoromethyl)phenyl) methanone | 404 | 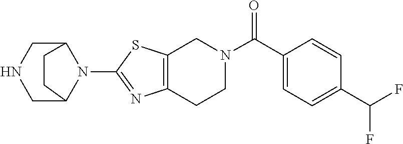 [SMILES: O=C(c1ccc(C(F)F)cc1)N1CCc2nc(N3C4CCC3CNC4)sc2C1] |
| Example 230 | (2-(3,8-diazabicyclo[3.2.1]octan-8- yl)-6,7-dihydrothiazolo[5,4-c]pyridin- 5(4H)-yl)(2,3-dihydrobenzofuran-7- yl)methanone | 396 | 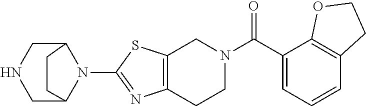 [SMILES: O=C(c1cccc2c1OCC2)N1CCc2nc(N3C4CCC3CNC4)sc2C1] |
| Example 231 | (2-(3,8-diazabicyclo[3.2.1]octan-8- yl)-6,7-dihydrothiazolo[5,4-c]pyridin- 5(4H)-yl)(4-(2,2-difluoroethyl) phenyl)methanone | 418 | 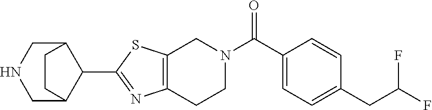 [SMILES: O=C(c1ccc(CC(F)F)cc1)N1CCc2nc(C3C4CCC3CNC4)sc2C1] |
| Example 232 | (2-(3,8-diazabicyclo[3.2.1]octan-8- yl)-6,7-dihydrothiazolo[5,4-c]pyridin- 5(4H)-yl)(4-(2,2,2-trifluoroethyl) phenyl)methanone | 436 | 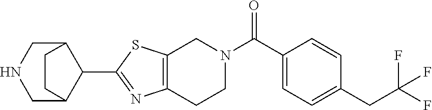 [SMILES: O=C(c1ccc(CC(F)(F)F)cc1)N1CCc2nc(C3C4CCC3CNC4)sc2C1] |
| Example 233 | (2-(3,8-diazabicyclo[3.2.1]octan-8- yl)-6,7-dihydrothiazolo[5,4-c]pyridin- 5(4H)-yl)(2,2-difluorobenzo[d][1,3] dioxol-4-yl)methanone | 434 | 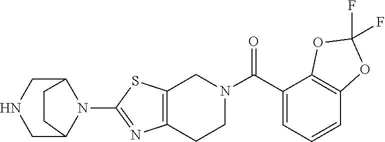 [SMILES: O=C(c1cccc2c1OC(F)(F)O2)N1CCc2nc(N3C4CCC3CNC4)sc2C1] |
| Example 234 | (2-(3,8-diazabicyclo[3.2.1]octan-8- yl)-6,7-dihydrothiazolo[5,4-c]pyridin- 5(4H)-yl)(benzo[d][1,3]dioxol-4- yl)methanone | 398 | 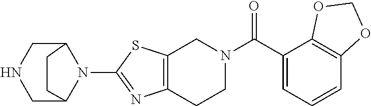 [SMILES: O=C(c1cccc2c1OCO2)N1CCc2nc(N3C4CCC3CNC4)sc2C1] |
| Example 235 | (2-(3,8-diazabicyclo[3.2.1]octan-8- yl)-6,7-dihydrothiazolo[5,4-c]pyridin- 5(4H)-yl)(3-fluorophenyl)methanone | 372 | 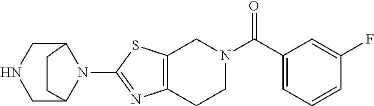 [SMILES: O=C(c1cccc(F)c1)N1CCc2nc(N3C4CCC3CNC4)sc2C1] |
| Example 236 | (2-(3,8-diazabicyclo[3.2.1]octan-8- yl)-6,7-dihydrothiazolo[5,4-c]pyridin- 5(4H)-yl)(2,3-dihydrobenzofuran-4- yl)methanone | 396 | 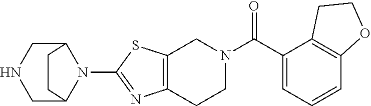 [SMILES: O=C(c1cccc2c1CCO2)N1CCc2nc(N3C4CCC3CNC4)sc2C1] |
| Example 237 | (2-(3,8-diazabicyclo[3.2.1]octan-8- yl)-6,7-dihydrothiazolo[5,4-c]pyridin- 5(4H)-yl)(quinolin-8-yl)methanone | 405 | 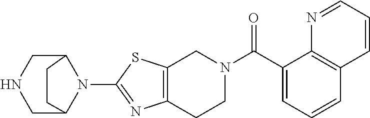 [SMILES: O=C(c1cccc2cccnc12)N1CCc2nc(N3C4CCC3CNC4)sc2C1] |
| Example 238 | 2-(2-(3,8-diazabicyclo[3.2.1]octan-8- yl)-4,5,6,7-tetrahydrothiazolo[5,4-c] pyridine-5-carbonyl)-5- methylbenzonitrile | 393 | 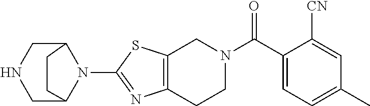 [SMILES: Cc1ccc(C(=O)N2CCc3nc(N4C5CCC4CNC5)sc3C2)c(C#N)c1] |
| Example 239 | 4-(2-(3,8-diazabicyclo[3.2.1]octan-8- yl)-4,5,6,7-tetrahydrothiazolo[5,4- c]pyridine-5-carbonyl)benzonitrile | 379 | 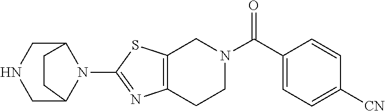 [SMILES: N#Cc1ccc(C(=O)N2CCc3nc(N4C5CCC4CNC5)sc3C2)cc1] |
| Example 240 | (2-(3,8-diazabicyclo[3.2.1]octan-8- yl)-6,7-dihydrothiazolo[5,4-c]pyridin- 5(4H)-yl)(6-fluoro-2-methoxypyridin- 3-yl)methanone | 403 | 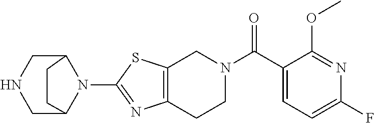 [SMILES: COc1nc(F)ccc1C(=O)N1CCc2nc(N3C4CCC3CNC4)sc2C1] |
| Example 241 | (2-(3,8-diazabicyclo[3.2.1]octan-8- yl)-6,7-dihydrothiazolo[5,4-c]pyridin- 5(4H)-yl)(6-(trifluoromethyl)pyridin- 3-yl)methanone | 423 | 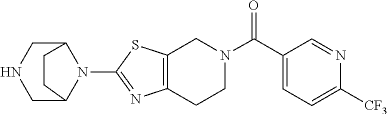 [SMILES: O=C(c1ccc(C(F)(F)F)nc1)N1CCc2nc(N3C4CCC3CNC4)sc2C1] |
| Example 242 | (2-(3,8-diazabicyclo[3.2.1]octan-8- yl)-6,7-dihydrothiazolo[5,4-c]pyridin- 5(4H)-yl)(6-fluoro-4-methylpyridin-3- yl)methanone | 387 | 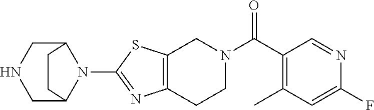 [SMILES: Cc1cc(F)ncc1C(=O)N1CCc2nc(N3C4CCC3CNC4)sc2C1] |
| Example 243 | (2-(3,8-diazabicyclo[3.2.1]octan-8- yl)-6,7-dihydrothiazolo[5,4-c]pyridin- 5(4H)-yl)(6-(difluoromethyl)pyridin-3- yl)methanone | 405 | 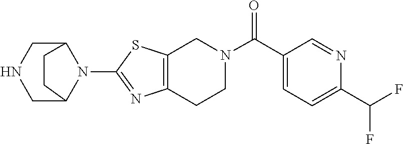 [SMILES: O=C(c1ccc(C(F)F)nc1)N1CCc2nc(N3C4CCC3CNC4)sc2C1] |
| Example 244 | cyclopentyl 2-(3,8- diazabicyclo[3.2.1]octan-8-yl)-6,7- dihydrothiazolo[5,4-c]pyridine-5(4H)- carboxylate | 362 | 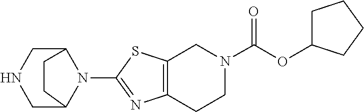 [SMILES: O=C(OC1CCCC1)N1CCc2nc(N3C4CCC3CNC4)sc2C1] |
| Example 245 | isopropyl 2-(3,8- diazabicyclo[3.2.1]octan-8-yl)-6,7- dihydrothiazolo[5,4-c]pyridine-5(4H)- carboxylate | 336 | 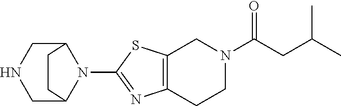 [SMILES: CC(C)CC(=O)N1CCc2nc(N3C4CCC3CNC4)sc2C1] |
| Example 246 | benzyl 2-(3,8- diazabicyclo[3.2.1]octan-8-yl)-6,7- dihydrothiazolo[5,4-c]pyridine-5(4H)- carboxylate | 384 | 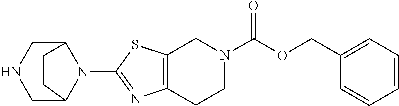 [SMILES: O=C(OCc1ccccc1)N1CCc2nc(N3C4CCC3CNC4)sc2C1] |
| Example 247 | phenyl 2-(3,8- diazabicyclo[3.2.1]octan-8- yl)-6,7-dihydrothiazolo[5,4-c] pyridine-5(4H)-carboxylate | 370 | 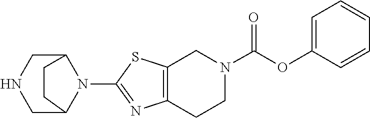 [SMILES: O=C(Oc1ccccc1)N1CCc2nc(N3C4CCC3CNC4)sc2C1] |
| Example 248 | 1-(2-(3,8-diazabicyclo[3.2.1]octan-8- yl)-6,7-dihydrothiazolo[5,4-c]pyridin- 5(4H)-yl)-2-hydroxy-2-(1- methylcyclopentyl)ethan-1-one | 390 | 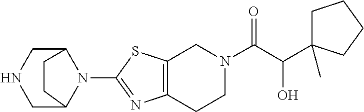 [SMILES: CC1(C(O)C(=O)N2CCc3nc(N4C5CCC4CNC5)sc3C2)CCCC1] |
| Example 249 | 1-(2-(3,8-diazabicyclo[3.2.1]octan-8- yl)-6,7-dihydrothiazolo[5,4-c]pyridin- 5(4H)-yl)-2-methoxy-2-(1- methylcyclopentyl)ethan-1-one | 404 | 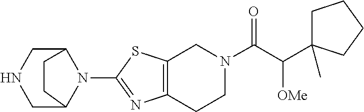 [SMILES: COC(C(=O)N1CCc2nc(N3C4CCC3CNC4)sc2C1)C1(C)CCCC1] |
| Example 250 | 1-(2-(3,8-diazabicyclo[3.2.1]octan-8- yl)-6,7-dihydrothiazolo[5,4-c]pyridin- 5(4H)-yl)-2-(2-(2- hydroxyethoxy)phenoxy) ethan-1-one | 444 | 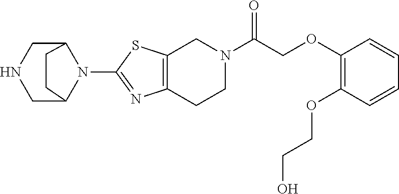 [SMILES: O=C(COc1ccccc1OCCO)N1CCc2nc(N3C4CCC3CNC4)sc2C1] |
| Example 251 | 1-(2-(3,8-diazabicyclo[3.2.1]octan-8- yl)-6,7-dihydrothiazolo[5,4-c]pyridin- 5(4H)-yl)-2,2-difluoro-2-(2- fluoropyridin-4-yl)ethan-1-one | 423 | 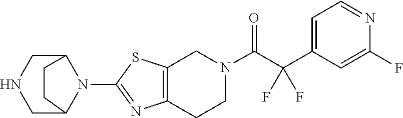 [SMILES: O=C(N1CCc2nc(N3C4CCC3CNC4)sc2C1)C(F)(F)c1ccnc(F)c1] |
| Example 252 | 1-(2-(3,8-diazabicyclo[3.2.1]octan-8- yl)-6,7-dihydrothiazolo[5,4-c]pyridin- 5(4H)-yl)-2-(2-(difluoromethoxy) phenoxy)ethan-1-one | 450 | 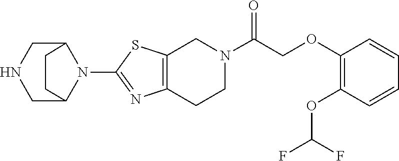 [SMILES: O=C(COc1ccccc1OC(F)F)N1CCc2nc(N3C4CCC3CNC4)sc2C1] |
| Example 253 | 2-(2-(2-(3,8-diazabicyclo[3.2.1]octan-8- yl)-6,7-dihydrothiazolo[5,4-c]pyridin- 5(4H)-yl)-2-oxoethoxy)-5- fluoro-3-methoxybenzonitrile | 457 | 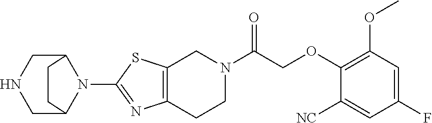 [SMILES: COc1cc(F)cc(C#N)c1OCC(=O)N1CCc2nc(N3C4CCC3CNC4)sc2C1] |
| Example 254 | 1-(2-(3,8-diazabicyclo[3.2.1]octan-8- yl)-6,7-dihydrothiazolo[5,4-c]pyridin- 5(4H)-yl)-2-cyclopentyl-2- hydroxypropan-1-one (enantiomer-1) | 390 | 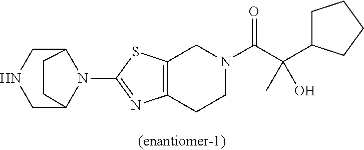 [SMILES: CC(O)(C(=O)N1CCc2nc(N3C4CCC3CNC4)sc2C1)C1CCCC1] |
| Example 255 | 1-(2-(3,8-diazabicyclo[3.2.1]octan-8- yl)-6,7-dihydrothiazolo[5,4-c]pyridin- 5(4H)-yl)-2-cyclopentyl-2- hydroxypropan-1-one (enantiomer-2) | 390 | 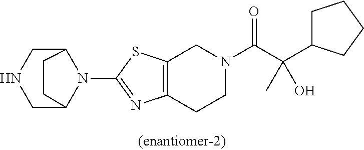 [SMILES: CC(O)(C(=O)N1CCc2nc(N3C4CCC3CNC4)sc2C1)C1CCCC1] |
| Example 256 | (2-(3,8-diazabicyclo[3.2.1]octan-8- yl)-7,7-dimethyl- 6,7-dihydrothiazolo[5,4-c]pyridin- 5(4H)-yl)(cyclopentyl) methanone | 374 | 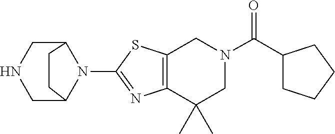 [SMILES: CC1(C)CN(C(=O)C2CCCC2)Cc2sc(N3C4CCC3CNC4)nc21] |
| Example 257 | 1-(2-(3,8-diazabicyclo[3.2.1]octan-8- yl)-7,7-dimethyl-6,7- dihydrothiazolo[5,4-c]pyridin-5(4H)- yl)-3-methylbutan-1-one | 362 | 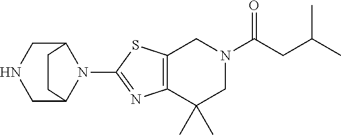 [SMILES: CC(C)CC(=O)N1Cc2sc(N3C4CCC3CNC4)nc2C(C)(C)C1] |
| Example 258 | 1-(2-(3,8-diazabicyclo[3.2.1]octan-8- yl)-7,7-dimethyl-6,7- dihydrothiazolo[5,4-c]pyridin- 5(4H)-yl)-2-methylpropan-1-one | 348 | 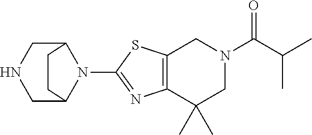 [SMILES: CC(C)C(=O)N1Cc2sc(N3C4CCC3CNC4)nc2C(C)(C)C1] |
| Example 259 | 1-(2-(3,8-diazabicyclo[3.2.1]octan-8- yl)-7,7-dimethyl-6,7- dihydrothiazolo[5,4-c]pyridin-5(4H)- yl)-2-hydroxy-3-methylbutan-1-one | 378 | 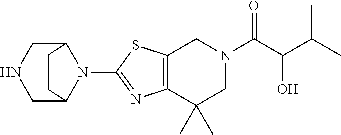 [SMILES: CC(C)C(O)C(=O)N1Cc2sc(N3C4CCC3CNC4)nc2C(C)(C)C1] |
| Example 260 | 1-(2-(3,8-diazabicyclo[3.2.1]octan-8- yl)-7,7-dimethyl-6,7- dihydrothiazolo[5,4-c]pyridin-5(4H)- yl)-2-(2-oxasprio[3.5]nonan-7- yl)ethan-1-one | 444 | 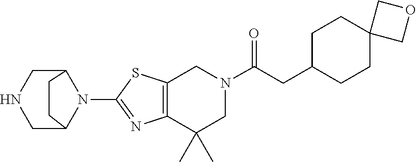 [SMILES: CC1(C)CN(C(=O)CC2CCC3(CC2)COC3)Cc2sc(N3C4CCC3CNC4)nc21] |
| Example 261 | 3-(2-(3,8-diazabicyclo[3.2.1]octan-8- yl)-7,7-dimethyl-6,7- dihydrothiazolo[5,4-c]pyridin-5(4H)- yl)-2-cyclopentyl-3-oxopropanenitrile | 413 | 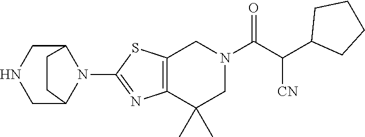 [SMILES: CC1(C)CN(C(=O)C(C#N)C2CCCC2)Cc2sc(N3C4CCC3CNC4)nc21] |
| Example 262 | (2R)-1-(2-(3,8- diazabicyclo[3.2.1]octan-8-yl)- 7,7-dimethyl-6,7-dihydrothiazolo [5,4-c]pyridin-5(4H)-yl)-2- cyclopentyl-2-hydroxyethan- 1-one | 404 | 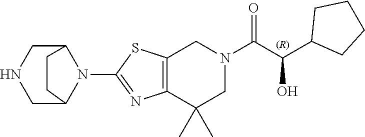 |
| Example 263 | (2-(3,8-diazabicyclo[3.2.1]octan-8- yl)-7,7-dimethyl-6,7- dihydrothiazolo[5,4-c]pyridin- 5(4H)-yl)(phenyl)methanone | 382 | 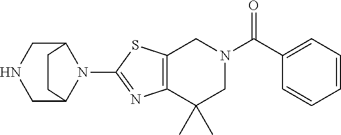 [SMILES: CC1(C)CN(C(=O)c2ccccc2)Cc2sc(N3C4CCC3CNC4)nc21] |
| Example 264 | (2-(3,8-diazabicyclo[3.2.1]octan-8- yl)-7,7-dimethyl-6,7-dihydrothiazolo [5,4-c]pyridin-5(4H)-yl)(6- fluoropyridin-3-yl)methanone | 401 | 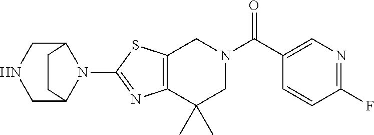 [SMILES: CC1(C)CN(C(=O)c2ccc(F)nc2)Cc2sc(N3C4CCC3CNC4)nc21] |
| Example 265 | 1-(2′-(3,8-diazabicyclo[3.2.1]octan-8- yl)-4′H-spiro[cyclopropane-1,7′- thiazolo[5,4-c]pyridin]-5′(6′H)-yl)-2- cyclopentylethan-1-one | 386 | 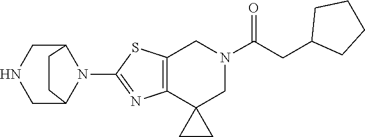 [SMILES: O=C(CC1CCCC1)N1Cc2sc(N3C4CCC3CNC4)nc2C2(CC2)C1] |
| Example 266 | 1-(2′-(3,8-diazabicyclo[3.2.1]octan-8- yl)-2,3,5,6-tetrahydro-4′H- spiro[pyran-4,7′-thiazolo[5,4- c]pyridin]-5′(6′H)-yl)-2- cyclopentylethan-1-one | 430 | 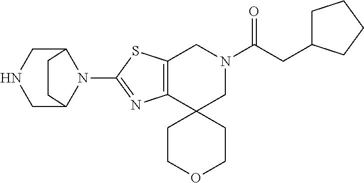 [SMILES: O=C(CC1CCCC1)N1Cc2sc(N3C4CCC3CNC4)nc2C2(CCOCC2)C1] |
| Example 267 | 1-(2-(3,8-diazabicyclo[3.2.1]octan-8- yl)-6,6-dimethyl-6,7- dihydrothiazolo[5,4-c]pyridin-5(4H)- yl)-2-cyclopentylethan-1-one | 388 | 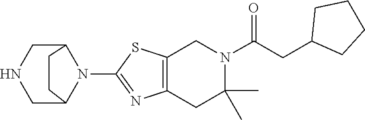 [SMILES: CC1(C)Cc2nc(N3C4CCC3CNC4)sc2CN1C(=O)CC1CCCC1] |
| Example 268 | 1-(2-(3,8-diazabicyclo[3.2.1]octan-8- yl)-6,6-dimethyl-6,7- dihydrothiazolo[5,4-c]pyridin-5(4H)- yl)-2-methylpropan-1-one | 348 | 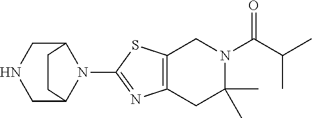 [SMILES: CC(C)C(=O)N1Cc2sc(N3C4CCC3CNC4)nc2CC1(C)C] |
| Example 269 | (2-(3,8-diazabicyclo[3.2.1]octan-8- yl)-6,6-dimethyl-6,7- dihydrothiazolo[5,4-c]pyridin-5(4H)- yl)(phenyl)methanone | 382 | 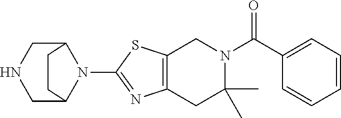 [SMILES: CC1(C)Cc2nc(N3C4CCC3CNC4)sc2CN1C(=O)c1ccccc1] |
| Example 270 | 1-(2-(3,8-diazabicyclo[3.2.1]octan-8- yl)-7,7-difluoro-6,7- dihydrothiazolo[5,4-c]pyridin-5(4H)- yl)-2-cyclopentylethan-1-one | 396 | 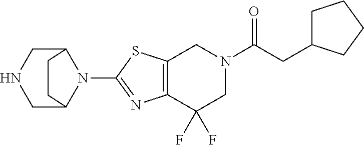 [SMILES: O=C(CC1CCCC1)N1Cc2sc(N3C4CCC3CNC4)nc2C(F)(F)C1] |
| Example 271 | (S)-2-cyclopentyl-1-(2- (hexahydropyrrolo[1,2-a]pyrazin- 2(1H)-yl)-6,7- dihydrothiazolo[5,4-c]pyridin-5(4H)- yl)ethan-1-one | 374 | 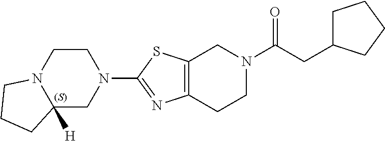 |
| Example 272 | (R)-2-cyclopentyl-1-(2- (hexahydropyrrolo[1,2-a]pyrazin- 2(1H)-6,7- dihydrothiazolo[5,4-c]pyridin-5(4H)- yl)ethan-1-one | 374 | 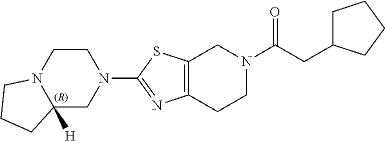 |
| Example 273 | 1-(2-(3,9-diazabicyclo[3.3.1]nonan-9- yl)-6,7- dihydrothiazolo[5,4-c]pyridin-5(4H)- yl)-2-cyclopentylethan-1-one | 374 | 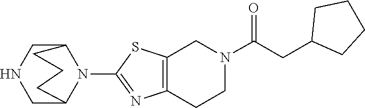 [SMILES: O=C(CC1CCCC1)N1CCc2nc(N3C4CCCC3CNC4)sc2C1] |
| Example 274 | 3,3-dimethyl-1-(2-(piperazin-1-yl)- 6,7-dihydrothiazolo[5,4-c]pyridin-5(4H)- yl)butan-1-one | 322 | 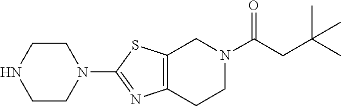 [SMILES: CC(C)(C)CC(=O)N1CCc2nc(N3CCNCC3)sc2C1] |
| Example 275 | (R)-2-cyclopentyl-1-(2-(2- (hydroxymethyl)piperazin-1-yl)- 6,7-dihydrothiazolo[5,4-c]pyridin-5(4H)- yl)ethan-1-one | 364 | 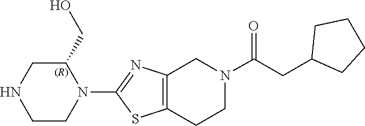 |
| Example 276 | 1-(2-(3,8-diazabicyclo[3.2.1]octan-8- yl)-6,7-dihydrothiazolo[5,4-c]pyridin-5(4H)- yl)-2-cyclopentylethan-1-one | 360 | 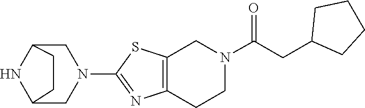 [SMILES: O=C(CC1CCCC1)N1CCc2nc(N3CC4CCC(C3)N4)sc2C1] |
| Example 277 | 1-(2-((1S,4S)-2,5- diazabicyclo[2.2.1]heptan-2-yl)- 6,7-dihydrothiazolo[5,4-c]pyridin-5(4H)- yl)-2-cyclopentylethan-1-one | 346 | 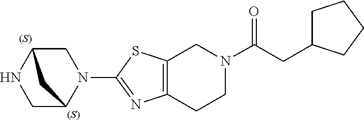 |
| Example 278 | 1-(2-(2,5-diazabicyclo[2.2.2]octan-2- yl)-6,7-dihydrothiazolo[5,4-c]pyridin-5(4H)- yl)-2-cyclopentylethan-1-one | 360 | 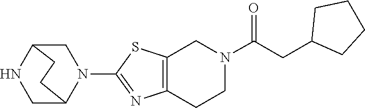 [SMILES: O=C(CC1CCCC1)N1CCc2nc(N3CC4CCC3CN4)sc2C1] |
| Example 279 | 1-(2-((1R,4R)-2,5- diazabicyclo[2.2.1]heptan-2-yl)- 6,7-dihydrothiazolo[5,4-c]pyridin-5(4H)- yl)-2-cyclopentylethan-1-one | 346 | 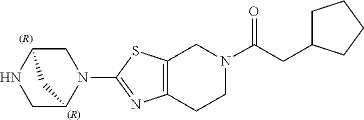 |
| Example 280 | 1-(2-(3,9-diazabicyclo[3.3.1]nonan-3- yl)-6,7-dihydrothiazolo[5,4-c]pyridin-5(4H)- yl)-2-cyclopentylethan-1-one | 374 | 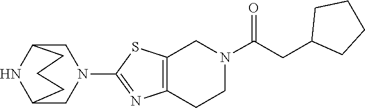 [SMILES: O=C(CC1CCCC1)N1CCc2nc(N3CC4CCCC(C3)N4)sc2C1] |
| Example 281 | 1-(2-(3,8-diazabicyclo[3.2.1]octan-8- yl)-6,7-dihydrothiazolo[5,4-c]pyridin-5(4H)- yl)-2-cyclopentylethan-1-one | 360 | 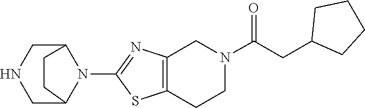 [SMILES: O=C(CC1CCCC1)N1CCc2sc(N3C4CCC3CNC4)nc2C1] |
| Example 282 | 1-(2-(3,8-diazabicyclo[3.2.1]octan-8- yl)-6,7-dihydrothiazolo[5,4-c]pyridin-5(4H)- yl)-3,3-dimethylbutan-1-one | 348 | 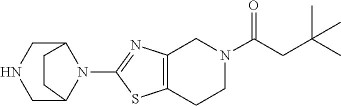 [SMILES: CC(C)(C)CC(=O)N1CCc2sc(N3C4CCC3CNC4)nc2C1] |
| Example 283 | 2-cyclopentyl-1-(2-(7-hydroxy-3,9- diazabicyclo[3.3.1]nonan-9-yl)- 6,7-dihydrothiazolo[5,4-c]pyridin-5(4H)- yl)ethan-1-one | 390 | 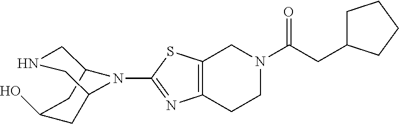 [SMILES: O=C(CC1CCCC1)N1CCc2nc(N3C4CNCC3CC(O)C4)sc2C1] |
| Example 284 | (S)-2-cyclopentyl-1-(2-(2- (hydroxymethyl)piperazin-1-yl)- 6,7-dihydrothiazolo[5,4-c]pyridin-5(4H)- yl)ethan-1-one | 364 | 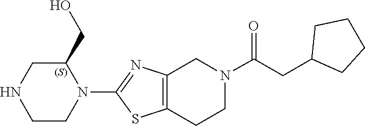 |
| Example 285 | (2-(3,8-diazabicyclo[3.2.1]octan-8- 5-((4-fluorobenzyl)sulfonyl)-4,5,6,7- tetrahydrothiazolo[5,4-c]pyridine | 422 | 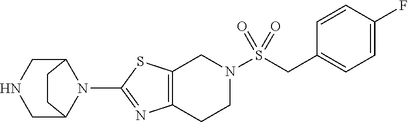 [SMILES: O=S(=O)(Cc1ccc(F)cc1)N1CCc2nc(N3C4CCC3CNC4)sc2C1] |
| Example 286 | 2-(3-ethyl-(3,8- diazabicyclo[3.2.1]octan-8- yl)-5-((4-fluorobenzyl)sulfonyl)- 4,5,6,7-tetrahydrothiazolo[5,4-c] pyridine | 450 | 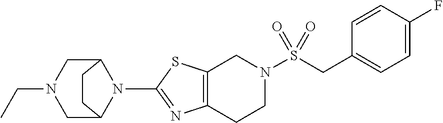 [SMILES: CCN1CC2CCC(C1)N2c1nc2c(s1)CN(S(=O)(=O)Cc1ccc(F)cc1)CC2] |
| Example 287 | 5-((4-fluorobenzyl)sulfonyl)-2- (piperazin-1-yl)-4,5,6,7- tetrahydrothiazolo[5,4-c]pyridine | 396 | 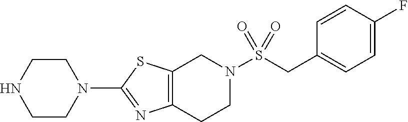 [SMILES: O=S(=O)(Cc1ccc(F)cc1)N1CCc2nc(N3CCNCC3)sc2C1] |
| Example 288 | 5-(benzylsulfonyl)-2- (3,8-diazabicyclo[3.2.1]octan-8- yl)-4,5,6,7-tetrahydrothiazolo [5,4-c]pyridine | 404 | 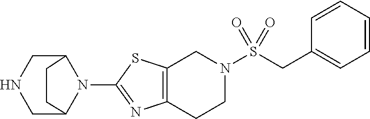 [SMILES: O=S(=O)(Cc1ccccc1)N1CCc2nc(N3C4CCC3CNC4)sc2C1] |
| Example 289 | 5-(benzylsulfonyl)-2-(3-ethyl- 3,8-diazabicyclo[3.2.1]octan-8- yl)-4,5,6,7-tetrahydrothiazolo [5,4-c]pyridine | 404 | 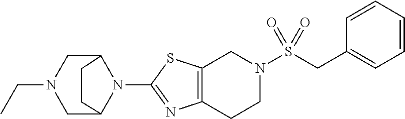 [SMILES: CCN1CC2CCC(C1)N2c1nc2c(s1)CN(S(=O)(=O)Cc1ccccc1)CC2] |
| Example 290 | 2-cyclopentyl-1-(2-(3-isopropyl- 3,8-diazabicyclo[3.2.1]octan-8- yl)-6,7-dihydrothiazolo[5,4-c]pyridin- 5(4H)-yl)ethan-1-one | 402 | 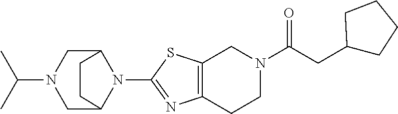 [SMILES: CC(C)N1CC2CCC(C1)N2c1nc2c(s1)CN(C(=O)CC1CCCC1)CC2] |
| Example 291 | 2-cyclopentyl-1-(2-(3-ethyl- 2-(3,8-diazabicyclo[3.2.1]octan-8-yl)- 6,7-dihydrothiazolo[5,4-c]pyridin- 5(4H)-yl)ethan-1-one | 388 | 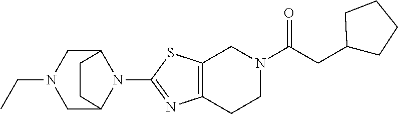 [SMILES: CCN1CC2CCC(C1)N2c1nc2c(s1)CN(C(=O)CC1CCCC1)CC2] |
| Example 292 | 2-cyclopentyl-1-(2-(3-propyl- 3,8-diazabicyclo[3.2.1]octan-8- yl)-6,7-dihydrothiazolo[5,4-c] pyridin-5(4H)-yl)ethan-1-one | 402 | 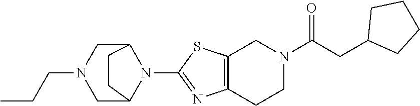 [SMILES: CCCN1CC2CCC(C1)N2c1nc2c(s1)CN(C(=O)CC1CCCC1)CC2] |
| Example 293 | 2-(3,8-diazabicyclo[3.2.1]octan-8-yl)- N-((S)-chroman-4-yl)- 6,7-dihydrothiazolo[5,4-c]pyridine- 5(4H)-carboxamide | 425 | 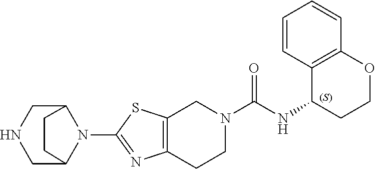 |
| Example 294 | N-((S)-chroman-4-yl)-2-(3-ethyl 3,8-diazabicyclo[3.2.1]octan-8- yl)-6,7-dihydrothiazolo[5,4-c]pyridine- 5(4H)-carboxamide | 453 | 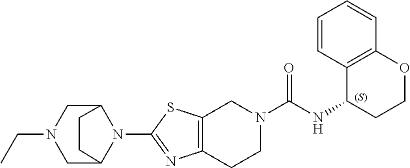 |
| Example 295 | N-((R)-chroman-4-yl)-2-(3-ethyl- 3,8-diazabicyclo[3.2.1]octan-8-yl)- 6,7-dihydrothiazolo[5,4-c]pyridine- 5(4H)-carboxamide | 453 | 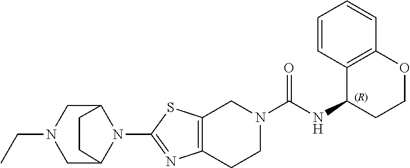 |
| Example 296 | (S)-N-(chroman-4-yl)-2-(piperazin-1- yl)-6,7-dihydrothiazolo[5,4-c] pyridine-5(4H)-carboxamide | 399 | 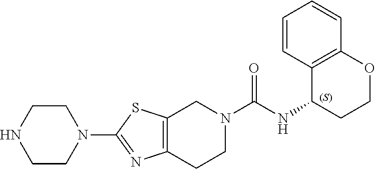 |
| Example 297 | (S)-N-(chroman-4-yl)-2-(4- ethylpiperazin-1-yl)-6,7- dihydrothiazolo[5,4-c]pyridine- 5(4H)-carboxamide | 427 | 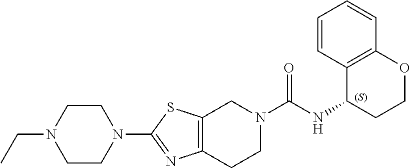 |
| Example 298 | 2-(4-fluorophenoxy)-1-(2-(3- isopropyl-3,8- diazabicyclo[3.2.1]octan-8-yl)- 6,7-dihydrothiazolo[5,4-c]pyridin-5(4H)- yl)ethan-1-one | 444 | 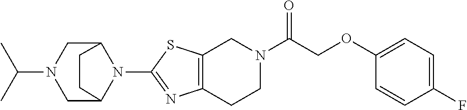 [SMILES: CC(C)N1CC2CCC(C1)N2c1nc2c(s1)CN(C(=O)COc1ccc(F)cc1)CC2] |
| Example 299 | 1-(2-(4-ethylpiperazin-1-yl)-6,7- dihydrothiazolo[5,4-c]pyridin-5(4H)- yl)-3,3-dimethylbutan-1-one | 350 | 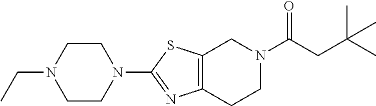 [SMILES: CCN1CCN(c2nc3c(s2)CN(C(=O)CC(C)(C)C)CC3)CC1] |
| Example 300 | 2-(3-ethyl-3,8- diazabicyclo[3.2.1]octan-8-yl)-N- (4-fluorobenzyl)-6,7- dihydrothiazolo[5,4-c]pyridine- 5(4H)-carboxamide | 429 | 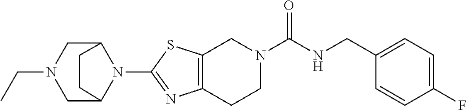 [SMILES: CCN1CC2CCC(C1)N2c1nc2c(s1)CN(C(=O)NCc1ccc(F)cc1)CC2] |
| Example 301 | 2-(4-fluorophenoxy)-1-(2-(3-methyl- 3,8-diazabicyclo[3.2.1]octan-8-yl)- 6,7-dihydrothiazolo[5,4-c]pyridin- 5(4H)-yl)ethan-1-one | 416 | 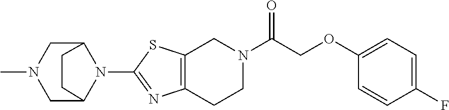 [SMILES: CN1CC2CCC(C1)N2c1nc2c(s1)CN(C(=O)COc1ccc(F)cc1)CC2] |
| Example 302 | 1-(2-(3-ethyl-3,8- diazabicyclo[3.2.1]octan-8-yl)- 6,7-dihydrothiazolo[5,4-c]pyridin-5(4H)- yl)-2-(4-fluorophenoxy)ethan-1-one | 430 | 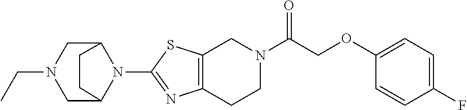 [SMILES: CCN1CC2CCC(C1)N2c1nc2c(s1)CN(C(=O)COc1ccc(F)cc1)CC2] |
| Example 303 | 2-(4-fluorophenoxy)-1-(2-(3-propyl- 3,8-diazabicyclo[3.2.1]octan-8-yl)- 6,7-dihydrothiazolo[5,4-c]pyridin-5(4H)- yl)ethan-1-one | 444 | 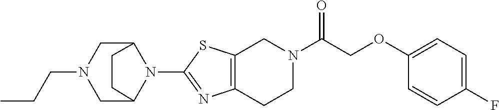 [SMILES: CCCN1CC2CCC(C1)N2c1nc2c(s1)CN(C(=O)COc1ccc(F)cc1)CC2] |
| Example 304 | 2-(4-ethylpiperazin-1-yl)-5-((4- fluorobenzyl)sulfonyl)-4,5,6,7- tetrahydrothiazolo[5,4-c]pyridine | 424 | 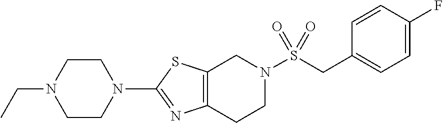 [SMILES: CCN1CCN(c2nc3c(s2)CN(S(=O)(=O)Cc2ccc(F)cc2)CC3)CC1] |
| Example 305 | 2-cyclopentyl-1-(2-(6-methylpyridin- 3-yl)-6,7-dihydrothiazolo[5,4-c]pyridin- 5(4H)-yl)ethan-1-one | 341 | 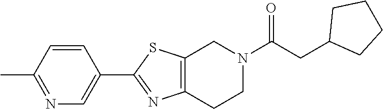 [SMILES: Cc1ccc(-c2nc3c(s2)CN(C(=O)CC2CCCC2)CC3)cn1] |
| Example 306 | 2-cyclopentyl-1-(2-(5-methylpyridin- 3-yl)-6,7-dihydrothiazolo[5,4-c]pyridin- 5(4H)-yl)ethan-1-one | 341 | 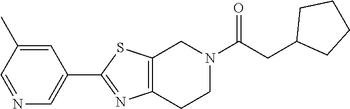 [SMILES: Cc1cncc(-c2nc3c(s2)CN(C(=O)CC2CCCC2)CC3)c1] |

TABLE 2
Enzyme assay data for PDE4D5, a long form of PDE4
and PDE4B2, a short form of PDE4
Using the method described in Experiment 1, the following PDE4 assay
data were obtained for exemplary compounds of the present invention.

| | % increase in PDE4D5 | % increase in PDE4B2 |
|---|---|---|
| Compound Example | (long isoform) activity | (short isoform) activity |
| Number | at 100 μM* | at 100 μM* |
| Example 108 | 22 | NT |
| Example 271 | 28 | −15 |

*Measured as mean % increase in counts over basal activity
NT: not tested

TABLE 3
cAMP levels in cultures of m-IMCD3 cells expressing PDE4 long forms
Using the method described in Experiment 2, the following m-IMCD3 cell
culture cAMP measurements were obtained for exemplary
compounds of the present invention.

| Compound Example | | | cAMP level (% of |
|---|---|---|---|
| Number | Concentration | | PGE2-stimulated)^{§} |
| Example 1 | 10 | μM | 14% |
| Example 10 | 10 | μM | 38% |
| Example 66 | 3 | μM | 36% |
| Example 68 | 3 | μM | 19% |
| Example 108 | 3 | μM | 53% |
| Example 118 | 3 | μM | 52% |
| Example 126 | 3 | μM | 39% |
| Example 127 | 3 | μM | 42% |
| Example 128 | 3 | μM | 50% |
| Example 171 | 3 | μM | 56% |
| Example 184 | 3 | μM | 58% |
| Example 241 | 3 | μM | 43% |
| Example 244 | 3 | μM | 40% |
| Example 257 | 3 | μM | 36% |
| Example 260 | 3 | μM | 52% |
| Example 268 | 3 | μM | 39% |
| Example 271 | 3 | μM | 42% |
| Example 273 | 3 | μM | 58% |
| Example 276 | 3 | μM | 41% |
| Example 277 | 3 | μM | 42% |
| Example 279 | 3 | μM | 27% |
| Example 280 | 3 | μM | 33% |
| Example 281 | 3 | μM | 51% |
| Example 282 | 3 | μM | 35% |
| Example 285 | 3 | μM | 35% |
| Example 286 | 3 | μM | 52% |
| Example 288 | 3 | μM | 34% |
| Example 290 | 3 | μM | 35% |
| Example 291 | 3 | μM | 45% |
| Example 293 | 3 | μM | 36% |
| Example 294 | 3 | μM | 39% |
| Example 297 | 3 | μM | 45% |
| Example 298 | 3 | μM | 48% |
| Example 300 | 3 | μM | 48% |
| Example 302 | 3 | μM | 48% |
| Example 303 | 3 | μM | 55% |
| Example 304 | 3 | μM | 47% |

^{§}Compared to 100% for (DMSO + PGE2)

TABLE 4
Inhibition of PGE2-stimulated in vitro cyst formation in m-IMCD3 cells
Using the method described in Experiment 3, the following m-IMCD3
kidney cell cyst suppression data were obtained for exemplary
compounds of the present invention.

| Compound | % Cyst area* after | Compound | % Cyst area* after |
|---|---|---|---|
| Example | incubation with | Example | incubation with |
| Number | 10 μM test compound | Number | 3 μM test compound |
| Example 1 | −5% | Example 129 | 3% |
| Example 2 | 2% | Example 157 | 17% |
| Example 3 | 13% | Example 158 | 7% |
| Example 4 | 12% | Example 159 | 0% |
| Example 5 | −5% | Example 160 | −2% |
| Example 9 | 0% | Example 161 | −3% |
| Example 10 | −3% | Example 162 | 14% |
| Example 11 | −3% | Example 164 | −7% |
| Example 12 | −2% | Example 165 | 2% |
| Example 16 | −2% | Example 167 | 19% |
| Example 19 | −2% | Example 168 | −3% |
| Example 20 | 9% | Example 169 | −9% |
| Example 21 | −1% | Example 170 | 5% |
| Example 22 | −2% | Example 171 | 6% |
| Example 23 | 0% | Example 172 | 10% |
| Example 24 | 0% | Example 173 | −2% |
| Example 25 | 6% | Example 174 | 3% |
| Example 26 | 1% | Example 175 | −5% |
| Example 28 | 3% | Example 176 | 12% |
| Example 30 | 1% | Example 177 | −2% |
| Example 31 | 7% | Example 178 | 24% |
| Example 32 | 1% | Example 179 | 2% |
| Example 33 | 8% | Example 180 | 2% |
| Example 34 | −2% | Example 181 | 5% |
| Example 37 | −2% | Example 182 | 1% |
| Example 38 | 8% | Example 183 | 0% |
| Example 51 | 9% | Example 184 | 15% |
| Example 52 | −6% | Example 185 | −2% |
| Example 55 | 0% | Example 186 | 0% |
| Example 56 | −6% | Example 187 | −8% |
| Example 57 | −3% | Example 188 | 3% |
| Example 58 | −3% | Example 189 | −1% |
| Example 60 | 2% | Example 190 | 1% |
| Example 62 | 1% | Example 192 | 1% |
| Example 65 | −2% | Example 193 | −1% |
| Example 66 | −5% | Example 194 | 1% |
| Example 67 | −4% | Example 195 | 1% |
| Example 68 | −4% | Example 196 | −1% |
| Example 70 | 7% | Example 197 | 4% |
| Example 71 | −3% | Example 198 | −5% |
| Example 72 | −3% | Example 199 | 3% |
| Example 73 | 3% | Example 200 | 4% |
| Example 75 | −4% | Example 201 | 17% |
| Example 77 | 1% | Example 202 | −4% |
| Example 79 | −2% | Example 203 | −1% |
| Example 82 | 2% | Example 204 | 4% |
| Example 83 | 4% | Example 205 | 0% |
| Example 84 | 1% | Example 206 | 6% |
| Example 85 | −2% | Example 207 | 0% |
| Example 86 | −1% | Example 208 | 8% |
| Example 97 | 6% | Example 209 | −5% |
| Example 98 | 3% | Example 210 | 11% |
| Example 100 | −1% | Example 211 | 40% |
| Example 102 | −3% | Example 212 | 0% |
| Example 103 | −1% | Example 213 | 17% |
| Example 104 | 0% | Example 214 | 54% |
| Example 107 | −16% | Example 215 | 7% |
| Example 109 | 6% | Example 216 | −3% |
| Example 110 | −5% | Example 217 | −3% |
| Example 116 | 55% | Example 218 | 11% |
| Example 117 | 1% | Example 219 | 11% |
| Example 118 | 5% | Example 220 | −5% |
| Example 119 | −10% | Example 221 | 0% |
| Example 120 | 2% | Example 222 | 4% |
| Example 121 | 0% | Example 223 | 14% |
| Example 122 | −8% | Example 224 | 22% |
| Example 123 | −4% | Example 225 | −1% |
| Example 124 | −12% | Example 228 | −6% |
| Example 125 | −11% | Example 229 | −1% |
| Example 126 | −10% | Example 230 | 22% |
| Example 127 | 3% | Example 232 | 5% |
| Example 128 | −2% | Example 233 | −2% |
| Example 130 | −5% | Example 234 | 1% |
| Example 131 | −9% | Example 235 | −3% |
| Example 132 | −6% | Example 236 | 27% |
| Example 133 | 19% | Example 237 | 37% |
| Example 134 | −4% | Example 238 | 5% |
| Example 135 | −6% | Example 239 | −2% |
| Example 136 | 18% | Example 240 | −3% |
| Example 137 | −4% | Example 241 | 4% |
| Example 138 | 0% | Example 242 | 29% |
| Example 139 | −1% | Example 243 | 10% |
| Example 140 | 0% | Example 244 | 6% |
| Example 141 | −7% | Example 247 | 18% |
| Example 142 | −4% | Example 249 | −5% |
| Example 143 | 2% | Example 250 | 17% |
| Example 144 | 13% | Example 251 | −6% |
| Example 145 | 17% | Example 253 | −4% |
| Example 146 | 16% | Example 254 | −5% |
| Example 147 | 9% | Example 255 | −5% |
| Example 148 | 3% | Example 257 | −3% |
| Example 149 | −2% | Example 259 | −6% |
| Example 150 | 1% | Example 260 | 10% |
| Example 151 | 5% | Example 262 | 23% |
| Example 152 | 4% | Example 264 | −1% |
| Example 153 | 31% | Example 265 | −3% |
| Example 154 | −13% | Example 268 | 1% |
| Example 155 | −3% | Example 270 | 2% |
| Example 156 | 0% | Example 271 | 4% |
| Example 266 | 40% | Example 272 | 16% |
| Example 284 | 19% | Example 273 | −11% |
| | | Example 274 | 14% |
| | | Example 275 | 19% |
| | | Example 276 | 13% |
| | | Example 277 | 6% |
| | | Example 278 | 2% |
| | | Example 279 | 19% |
| | | Example 280 | −10% |
| | | Example 281 | −9% |
| | | Example 282 | 3% |
| | | Example 285 | −3% |
| | | Example 286 | −2% |
| | | Example 287 | −3% |
| | | Example 288 | 7% |
| | | Example 289 | −7% |
| | | Example 290 | −9% |
| | | Example 291 | 3% |
| | | Example 292 | 0% |
| | | Example 293 | 4% |
| | | Example 294 | −5% |
| | | Example 295 | −4% |
| | | Example 296 | 33% |
| | | Example 297 | 9% |
| | | Example 298 | −7% |
| | | Example 299 | 3% |
| | | Example 300 | −3% |
| | | Example 301 | −6% |
| | | Example 302 | 5% |
| | | Example 303 | 0% |
| | | Example 304 | 13% |
| | | Example 305 | 26% |
| | | Example 306 | −3% |

*Compared to 100% for (DMSO + PGE2) and 0% for DMSO control

It will be appreciated that the above description is made by way of example and not limitation of the scope of the appended claims, including any equivalents as included within the scope of the claims. Various modifications are possible and will be readily apparent to the skilled person in the art. Likewise, features of the described embodiments can be combined with any appropriate aspect described above and optional features of any one aspect can be combined with any other appropriate aspect.

1. A compound of Formula I 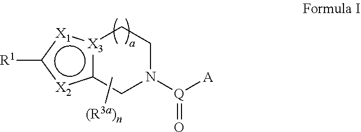
or a pharmaceutically acceptable salt or derivative thereof, wherein:
one of X₁ and X₂ is N and the other is S, N, or CR^{3b}, X₃ is N or C;
Q is C or S(O);
R¹ is a 4- to 10-membered non-aromatic ring system that may be monocyclic, bridged or bicyclic, containing at least 1 ring N heteroatom and optionally a ring O heteroatom, and wherein R¹ is optionally substituted with 1 or more R⁴;
A is R^{2c}, NR^{2a}R^{2b}, or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1;
for use in the treatment or prevention of a disease or disorder that can be ameliorated by activation of long isoforms of PDE4 or a disease or disorder mediated by excessive intracellular cyclic AMP signalling.

2. The compound for use of claim 1, where in the compound is a compound of formula: 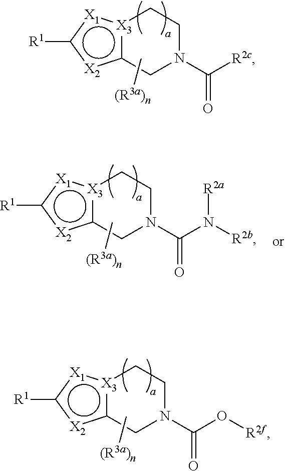
or a pharmaceutically acceptable salt or derivative thereof.

3. The compound, or a pharmaceutically acceptable salt or derivative thereof, for use of claim 1 or 2, wherein R¹ is a 5- to 6-membered saturated, monocyclic ring containing at least 1 ring N heteroatom and optionally a ring O heteroatom; a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.

4. The compound, or a pharmaceutically acceptable salt or derivative thereof, for use of any preceding claim, wherein R¹ is a 4- to 10-membered non-aromatic ring that may be monocyclic, bridged or bicyclic containing at least 1 ring N heteroatom and optionally a ring O heteroatom; wherein R¹ is optionally substituted with 1 R⁴.

5. The compound, or a pharmaceutically acceptable salt or derivative thereof, for use of any one of claims 1 to 3, wherein R¹ is a 7- to 9-membered saturated, bridged ring system containing 2 ring N heteroatoms, optionally a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

6. The compound, or a pharmaceutically acceptable salt or derivative thereof, for use of any preceding claim, wherein A is R^{2c} or NR^{2a}R^{2b}.

7. The compound, or a pharmaceutically acceptable salt or derivative thereof, for use of any preceding claim, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered (optionally 5- to 7-membered) non-aromatic heterocycle, optionally containing 1 further heteroatom selected from O, and optionally substituted with 1 or more R⁵; or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵; or
c) R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵.

8. The compound, or a pharmaceutically acceptable salt or derivative thereof, for use of any preceding claim, wherein:
a) R^{2a} is a (C5-10)alkyl group comprising a cyclic moiety; and wherein R^{2a} is optionally substituted with 1 or more R⁵; and R^{2b} is H or (C1-6)alkyl, and wherein (C1-6)alkyl is optionally substituted with 1 or more R⁵; or
b) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain or branched; and wherein R^{2a} is substituted with 1 or more R⁵ (optionally wherein R⁵ is halogen); and R^{2b} is H or (C1-6)alkyl, and wherein (C1-6)alkyl is optionally substituted with 1 or more R⁵.

9. The compound, or a pharmaceutically acceptable salt or derivative thereof, for use of any of claims 1 to 7, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain or branched, wherein a C atom in straight chain portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; or
6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0 or 1 ring N atoms; and wherein R^{2c} is substituted with 1 or more R⁵ (optionally wherein R⁵ is halogen).

10. The compound, or a pharmaceutically acceptable salt or derivative thereof, for use of any preceding claim, wherein the compound is of formula: 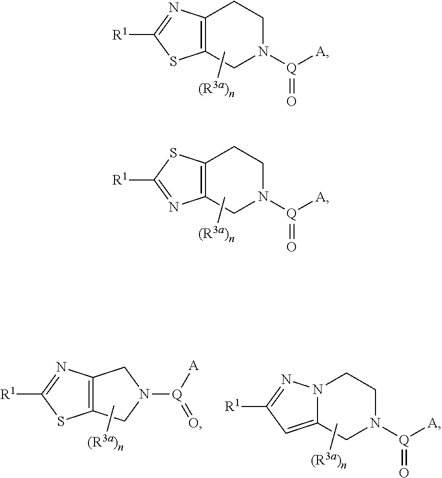
or a pharmaceutically acceptable salt or derivative thereof.

11. The compound, or a pharmaceutically acceptable salt or derivative thereof, for use of any preceding claim, wherein each R^{3a} is —CH₃ or F, or two R^{3a} attached to the same carbon are joined together with the atom to which they are attached to form a 3- to 6-membered cycloalkyl ring, or 5- to 6-membered heterocycloalky ring containing an O heteroatom (optionally a cyclopropyl ring).

12. The compound, or a pharmaceutically acceptable salt or derivative thereof, for use of any preceding claim, wherein n is 0, 1 or 2, optionally wherein n is 2 and both R^{3a} substituents appear on the same ring carbon atom.

13. The compound, or a pharmaceutically acceptable salt or derivative thereof, for use of any preceding claim, wherein n is 0.

14. The compound, or a pharmaceutically acceptable salt or derivative thereof, for use of any preceding claim, wherein one of X₁ and X₂ is N and the other is S, and X₃ is C.

15. The compound, or a pharmaceutically acceptable salt or derivative thereof, for use of any preceding claim, wherein Q is C and/or A is R^{2c}.

16. The compound, or a pharmaceutically acceptable salt or derivative thereof, for use of any preceding claim, wherein
Q is C;
R¹ is a 7- to 9-membered saturated, bridged ring system containing 2 ring N heteroatoms, wherein R¹ is optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

17. A compound of Formula II: 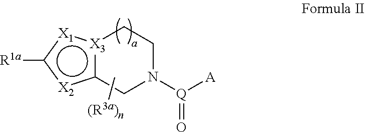
or a pharmaceutically acceptable salt or derivative thereof, wherein:
one of X₁ and X₂ is N and the other is S, N, or CR^{3b}, X₃ is N or C;
Q is C or S(O);
R^{1a} is a 7- to 9-membered saturated, bridged ring system containing 2 ring N heteroatoms, and wherein R^{1a} is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocyclic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1.

18. The compound, or a pharmaceutically acceptable salt or derivative thereof, of claim 17, wherein R^{1a} is a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

19. The compound, or a pharmaceutically acceptable salt or derivative thereof, of any claim 17 or 18, wherein the compound is of formula: 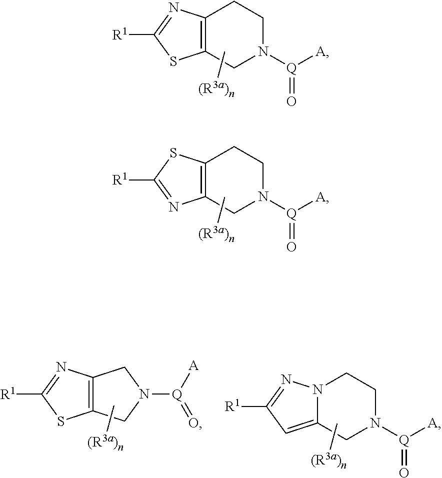
or a pharmaceutically acceptable salt or derivative thereof.

20. The compound, or a pharmaceutically acceptable salt or derivative thereof, of any of claims 17-19, wherein one of X₁ and X₂ is N and the other is S and X₃ is C.

21. A compound of Formula III: 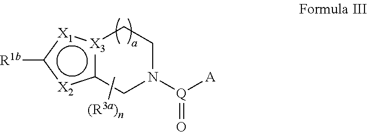
or a pharmaceutically acceptable salt or derivative thereof, wherein:
one of X₁ and X₂ is N and the other is S, N, or CR^{3b}, X₃ is N or C;
Q is C or S(O);
R^{1b} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1b}, and wherein R^{1b} is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together to form a 3- to 6-membered carbocyclic ring or heterocyclic ring containing an O heteroatom, optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
n is 0, 1, 2, 3 or 4; and
a is 0 or 1;
wherein when one of X₁ and X₂ is N and the other is S, X₃ is C and (a) A is R^{2c} or NR^{2a}R^{2b}, then R^{1b} is not 4-methyl-piperazin-1-yl or 4-ethyl-piperazin-1yl or (b) A is OR^{2f}, then R^{1b} is not a monocyclic piperazine ring.

22. The compound or a pharmaceutically acceptable salt or derivative thereof of claim 21, wherein R^{1b} is a 5- to 6-membered saturated, monocyclic ring containing at least 1 ring N heteroatom and optionally a ring O heteroatom; or a 7- to 9-membered saturated, bridged ring system containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R^{1b} is optionally substituted with 1, 2 or 3 R⁴.

23. The compound, or a pharmaceutically acceptable salt or derivative thereof, of claim 21 or 22, wherein R^{1b} is a 7- to 9-membered saturated, bridged ring system containing 2 ring N heteroatoms; wherein R^{1a} is optionally substituted with 1 R⁴, optionally wherein R^{1a} is a 7- to 8-membered saturated, bridged ring system containing 2 ring N heteroatoms (for example a bridged piperazine, such as 3,8-diazabicyclo[3.2.1]octanyl), wherein R^{1b} is optionally substituted with 1 R⁴.

24. The compound, or a pharmaceutically acceptable salt or derivative thereof, of any of claims 17-23, wherein:
a) R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group (optionally a (C3-10)alkyl group) that may be straight chain, branched or cyclic, or a combination thereof; and wherein R^{2a} is optionally substituted with 1 or more R⁵; and
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered (optionally 5- to 7-membered) non-aromatic heterocycle, optionally containing 1 further heteroatom selected from O, and optionally substituted with 1 or more R⁵; or
b) R^{2c} is CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵; or
c) R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; and wherein R^{2f} is optionally substituted with 1 or more R⁵.

25. The compound, or a pharmaceutically acceptable salt or derivative thereof, of any of claims 17-24, wherein R^{2c} is:
a) a (C3-10)alkyl group comprising a cyclic moeity, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵;
b) CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; or a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; or 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is substituted with 1 or more R⁵ (optionally wherein R⁵ is halogen).

26. The compound, or a pharmaceutically acceptable salt or derivative thereof, of any of claims 17-25, wherein the compound is of formula: 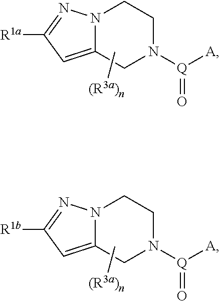
or a pharmaceutically acceptable salt or derivative thereof.

27. The compound, or a pharmaceutically acceptable salt or derivative thereof, of any of claims 17-26, wherein Q is C and/or A is R^{2c}.

28. The compound, or a pharmaceutically acceptable salt or derivative thereof, of any of claims 17-26, wherein
Q is C;
R^{1a} or R^{1b} is a 7- to 9-membered saturated, bridged ring system containing 2 ring N heteroatoms, wherein R^{1a} and R^{1b} are optionally substituted with 1 R⁴;
A is R^{2c};
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group maybe substituted with a 6-membered aromatic or heteroaromatic ring that contains 0, 1 or 2 ring N atoms; and wherein R^{2c} is optionally substituted with 1 or more R⁵
R^{3a}, where present, is methyl;
R⁴, where present, is (C1-6)alkyl optionally substituted with OH, optionally (C1-2)alkyl optionally substituted with OH;
R⁵, where present, is OH or halo; and
n is 0, 1 or 2.

29. The compound, or a pharmaceutically acceptable salt or derivative thereof, of any of claims 17-28, wherein each R^{3a} is —CH₃, or two R^{3a} attached to the same carbon are joined together with the atoms to which they are attached to form a cyclopropyl ring.

30. The compound, or a pharmaceutically acceptable salt or derivative thereof, of any of claims 17-29, wherein n is 0, 1 or 2.

31. The compound, or a pharmaceutically acceptable salt or derivative thereof, of any of claims 17-30, wherein n is 0.

32. A compound of Formula V: 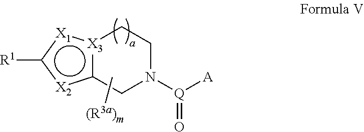
or a pharmaceutically acceptable salt or derivative thereof, wherein:
one of X₁ and X₂ is N and the other is S, N, or CR^{3b}, X₃ is N or C;
Q is C or S(O);
R¹ is a 4- to 10-membered non-aromatic ring system that may be monocyclic, bridged or bicyclic containing at least 1 ring N heteroatom and optionally a ring O heteroatom, and wherein R¹ is optionally substituted with 1 or more R⁴;
A is R^{2c}, NR^{2a}R^{2b} or OR^{2f};
R^{2a} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2a} is optionally substituted with 1 or more R⁵;
R^{2b} is H or (C1-6)alkyl, and wherein (C1-6)alkyl is optionally substituted with 1 or more R⁵; or
R^{2a} and R^{2b}, together with the N atom to which they are attached, form a 5- to 10-membered non-aromatic heterocycle, optionally containing 1 further heteroatom selected from O, and optionally substituted with 1 or more R⁵;
R^{2c} is a (C3-10)alkyl group that may be straight chain, branched or cyclic, or a combination thereof, wherein a C atom in straight chain or cyclic portion of said (C3-10)alkyl group may be optionally replaced by 1 —O— other than at the point of attachment of R^{2c}, wherein said (C3-10)alkyl group may be substituted with a 6-membered aromatic or heteroaromatic ring that contains 0, 1 or 2 ring N atoms; CH₂-[6-membered aromatic or heteroaromatic ring that contains 0, 1 or 2 ring N atoms]; CH₂—O-[6-membered aromatic or heteroaromatic ring that contains 0, 1 or 2 ring N atoms]; 6-membered aromatic or heteroaromatic ring that contains 0, 1 or 2 ring N atoms; or a 9- or 10-membered bicyclic ring system comprising a 5- to 6-membered aromatic or non-aromatic heterocycle containing 1, 2 or 3 ring N or O heteroatoms fused to a 6-membered aromatic or heteroaromatic ring that contains 0, 1 or 2 ring N atoms; and wherein R^{2c} is optionally substituted with 1 or more R⁵;
R^{2f} is a 6-membered aromatic or heteroaromatic ring that contains 0, 1 or 2 ring N atoms; CH₂-[6-membered aromatic or heteroaromatic ring that contains 0, 1 or 2 ring N atoms]; or a (C2-10)alkyl group that may be straight chain, branched or cyclic, or a combination thereof; (C5-7)cycloalkyl fused to a 6-membered aromatic or heteroaromatic ring that contains 0, 1 or 2 ring N atoms; or a 5- to 7-membered non-aromatic heterocycle containing one ring O heteroatom, optionally fused to a 6-membered aromatic or heteroaromatic ring that contains 0, 1 or 2 ring N atoms; and wherein R^{2f} is optionally substituted with 1 or more R⁵;
each R^{3a} is independently (C1-6)alkyl or fluoro, the (C1-6)alkyl being optionally substituted by 1 or more halogen; or
two R^{3a} attached to the same or adjacent carbon atoms may be joined together with the atoms to which they are attached to form a 3- to 6-membered carbocyclic ring or heterocyclic ring containing an O heteroatom, wherein said ring is optionally substituted by 1 or more halogen;
each R^{3b} is independently H or (C1-6)alkyl;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen, OH or (C1-6)alkoxy;
m is 1, 2, 3 or 4; and
a is 0 or 1.

33. The compound, or a pharmaceutically acceptable salt or derivative thereof, of any of claims 17-32, wherein A is R^{2c} or NR^{2a}R^{2b}.

34. A compound selected from:
1-(2-(3,8-diazabicyclo[3.2.1]octan-8-yl)-6,7-dihydrothiazolo[5,4-c]pyridin-5(4H)-yl)-2-cyclopentylethan-1-one;
1-(2-(3,8-diazabicyclo[3.2.1]octan-8-yl)-6,7-dihydrothiazolo[5,4-c]pyridin-5(4H)-yl)-2-(4,4-difluorocyclohexyl)ethan-1-one;
1-(2-(3,8-diazabicyclo[3.2.1]octan-8-yl)-6,7-dihydrothiazolo[5,4-c]pyridin-5(4H)-yl)-2-(tetrahydro-2H-pyran-4-yl)ethan-1-one;
1-(2-(3,8-diazabicyclo[3.2.1]octan-8-yl)-6,7-dihydrothiazolo[5,4-c]pyridin-5(4H)-yl)-2-isopropoxyethan-1-one;
1-(2-(3,8-diazabicyclo[3.2.1]octan-8-yl)-6,7-dihydrothiazolo[5,4-c]pyridin-5(4H)-yl)-3-phenylpropan-1-one;
1-(2-(3,8-diazabicyclo[3.2.1]octan-8-yl)-6,7-dihydrothiazolo[5,4-c]pyridin-5(4H)-yl)-2-phenoxyethan-1-one;
4-(3-(2-(3,8-diazabicyclo[3.2.1]octan-8-yl)-6,7-dihydrothiazolo[5,4-c]pyridin-5(4H)-yl)-3-oxopropyl)benzonitrile;
1-(2-(3,8-diazabicyclo[3.2.1]octan-8-yl)-6,7-dihydrothiazolo[5,4-c]pyridin-5(4H)-yl)-2-phenylethan-1-one;
1-(2-(3,8-diazabicyclo[3.2.1]octan-8-yl)-6,7-dihydrothiazolo[5,4-c]pyridin-5(4H)-yl)-3-methylbutan-1-one;
1-(2-(3,8-diazabicyclo[3.2.1]octan-8-yl)-6,7-dihydrothiazolo[5,4-c]pyridin-5(4H)-yl)-3,3-dimethylbutan-1-one;
1-(2-(3,8-diazabicyclo[3.2.1]octan-8-yl)-6,7-dihydrothiazolo[5,4-c]pyridin-5(4H)-yl)-2-cyclobutylethan-1-one;
1-(2-(3,8-diazabicyclo[3.2.1]octan-8-yl)-6,7-dihydrothiazolo[5,4-c]pyridin-5(4H)-yl)-2-(bicyclo[1.1.1]pentan-1-yl)ethan-1-one;
1-(2-(3,8-diazabicyclo[3.2.1]octan-8-yl)-6,7-dihydrothiazolo[5,4-c]pyridin-5(4H)-yl)-2-cyclohexylethan-1-one;
(2-(3,8-diazabicyclo[3.2.1]octan-8-yl)-6,7-dihydrothiazolo[5,4-c]pyridin-5(4H)-yl)(4,4-difluorocyclohexyl)methanone;
(2S)-1-(2-(3,8-diazabicyclo[3.2.1]octan-8-yl)-6,7-dihydrothiazolo[5,4-c]pyridin-5(4H)-yl)-2-methylbutan-1-one;
(2R)-1-(2-(3,8-diazabicyclo[3.2.1]octan-8-yl)-6,7-dihydrothiazolo[5,4-c]pyridin-5(4H)-yl)-2-methylbutan-1-one;
1-(2-(3,8-diazabicyclo[3.2.1]octan-8-yl)-6,7-dihydrothiazolo[5,4-c]pyridin-5(4H)-yl)-2,3-dimethylbutan-1-one;
(2S)-1-(2-(3,8-diazabicyclo[3.2.1]octan-8-yl)-6,7-dihydrothiazolo[5,4-c]pyridin-5(4H)-yl)-2,3-dimethylbutan-1-one;
(2R)-1-(2-(3,8-diazabicyclo[3.2.1]octan-8-yl)-6,7-dihydrothiazolo[5,4-c]pyridin-5(4H)-yl)-2,3-dimethylbutan-1-one;
1-(2-(3,8-diazabicyclo[3.2.1]octan-8-yl)-6,7-dihydrothiazolo[5,4-c]pyridin-5(4H)-yl)-2-methylpropan-1-one;
1-(2-(3,8-diazabicyclo[3.2.1]octan-8-yl)-6,7-dihydrothiazolo[5,4-c]pyridin-5(4H)-yl)-2-(4-fluorophenoxy)ethan-1-one;
(2-(3,8-diazabicyclo[3.2.1]octan-8-yl)-6,7-dihydrothiazolo[5,4-c]pyridin-5(4H)-yl)(cyclopentyl)methanone;
1-(2-(3,8-diazabicyclo[3.2.1]octan-8-yl)-6,7-dihydrothiazolo[5,4-c]pyridin-5(4H)-yl)-2-phenoxypropan-1-one;
1-(2-(3,8-diazabicyclo[3.2.1]octan-8-yl)-6,7-dihydrothiazolo[5,4-c]pyridin-5(4H)-yl)-2-cyclopentyl-2,2-difluoroethan-1-one;
1-(2-(3,8-diazabicyclo[3.2.1]octan-8-yl)-6,7-dihydrothiazolo[5,4-c]pyridin-5(4H)-yl)-2-(4-fluorophenyl)ethan-1-one;
1-(2-(3,8-diazabicyclo[3.2.1]octan-8-yl)-6,7-dihydrothiazolo[5,4-c]pyridin-5(4H)-yl)-3,3,3-trifluoropropan-1-one;
1-(2-(3,8-diazabicyclo[3.2.1]octan-8-yl)-6,7-dihydrothiazolo[5,4-c]pyridin-5(4H)-yl)-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-hydroxy-3-methylbutan-1-one;
(2-(3,8-diazabicyclo[3.2.1]octan-8-yl)-6,7-dihydrothiazolo[5,4-c]pyridin-5(4H)-yl)(3,3-difluorocyclopentyl)methanone;
1-(2-(3,8-diazabicyclo[3.2.1]octan-8-yl)-6,7-dihydrothiazolo[5,4-c]pyridin-5(4H)-yl)-2-methyl-2-phenoxypropan-1-one;
(2-(3,8-diazabicyclo[3.2.1]octan-8-yl)-6,7-dihydrothiazolo[5,4-c]pyridin-5(4H)-yl)(1-phenoxycyclopropyl)methanone;
1-(2-(3,8-diazabicyclo[3.2.1]octan-8-yl)-6,7-dihydrothiazolo[5,4-c]pyridin-5(4H)-yl)-3,3,3-trifluoro-2,2-dimethylpropan-1-one;
(2-(3,8-diazabicyclo[3.2.1]octan-8-yl)-6,7-dihydrothiazolo[5,4-c]pyridin-5(4H)-yl)(1-phenylcyclopropyl)methanone;
1-(2-(3,8-diazabicyclo[3.2.1]octan-8-yl)-6,7-dihydrothiazolo[5,4-c]pyridin-5(4H)-yl)-2-cyclopentyl-2-fluoroethan-1-one;
1-(2-(3,8-diazabicyclo[3.2.1]octan-8-yl)-6,7-dihydrothiazolo[5,4-c]pyridin-5(4H)-yl)-2-(4,4-difluorocyclohexyl)-2-fluoroethan-1-one;
1-(2-(3,8-diazabicyclo[3.2.1]octan-8-yl)-6,7-dihydrothiazolo[5,4-c]pyridin-5(4H)-yl)-2,2-difluoro-2-phenylethan-1-one;
(2-(3,8-diazabicyclo[3.2.1]octan-8-yl)-6,7-dihydrothiazolo[5,4-c]pyridin-5(4H)-yl)(2,2-dimethylcyclopentyl)methanone;
(2-(3,8-diazabicyclo[3.2.1]octan-8-yl)-6,7-dihydrothiazolo[5,4-c]pyridin-5(4H)-yl)(3,3-dimethylcyclopentyl)methanone;
2-(3,8-diazabicyclo[3.2.1]octan-8-yl)-N-cyclopentyl-6,7-dihydrothiazolo[5,4-c]pyridine-5(4H)-carboxamide;
2-(3,8-diazabicyclo[3.2.1]octan-8-yl)-N-isopropyl-6,7-dihydrothiazolo[5,4-c]pyridine-5(4H)-carboxamide;
2-(3,8-diazabicyclo[3.2.1]octan-8-yl)-N-cyclohexyl-6,7-dihydrothiazolo[5,4-c]pyridine-5(4H)-carboxamide;
2-(3,8-diazabicyclo[3.2.1]octan-8-yl)-N-cycloheptyl-6,7-dihydrothiazolo[5,4-c]pyridine-5(4H)-carboxamide;
2-(3,8-diazabicyclo[3.2.1]octan-8-yl)-N-(cyclohexylmethyl)-6,7-dihydrothiazolo[5,4-c]pyridine-5(4H)-carboxamide;
2-(3,8-diazabicyclo[3.2.1]octan-8-yl)-N-(4-fluorobenzyl)-6,7-dihydrothiazolo[5,4-c]pyridine-5(4H)-carboxamide;
2-(3,8-diazabicyclo[3.2.1]octan-8-yl)-N-(4-(trifluoromethyl)benzyl)-6,7-dihydrothiazolo[5,4-c]pyridine-5(4H)-carboxamide;
2-(3,8-diazabicyclo[3.2.1]octan-8-yl)-N-phenyl-6,7-dihydrothiazolo[5,4-c]pyridine-5(4H)-carboxamide;
2-(3,8-diazabicyclo[3.2.1]octan-8-yl)-N-(4,4-difluorocyclohexyl)-6,7-dihydrothiazolo[5,4-c]pyridine-5(4H)-carboxamide;
(2-(3,8-diazabicyclo[3.2.1]octan-8-yl)-6,7-dihydrothiazolo[5,4-c]pyridin-5(4H)-yl)(pyrrolidin-1-yl)methanone;
2-(3,8-diazabicyclo[3.2.1]octan-8-yl)-N-cyclopentyl-N-methyl-6,7-dihydrothiazolo[5,4-c]pyridine-5(4H)-carboxamide;
1-(2-(3,8-diazabicyclo[3.2.1]octan-8-yl)-6,7-dihydrothiazolo[5,4-c]pyridin-5(4H)-yl)-2,2-dimethylpropan-1-one;
1-(2-(3,8-diazabicyclo[3.2.1]octan-8-yl)-4,6-dihydro-5H-pyrrolo[3,4-d]thiazol-5-yl)-2-cyclopentylethan-1-one;
1-(2-(3,8-diazabicyclo[3.2.1]octan-8-yl)-4,6-dihydro-5H-pyrrolo[3,4-d]thiazol-5-yl)-2-(4,4-difluorocyclohexyl)ethan-1-one;
2-(3,8-diazabicyclo[3.2.1]octan-8-yl)-N-cyclopentyl-4,6-dihydro-5H-pyrrolo[3,4-d]thiazole-5-carboxamide;
2-(3,8-diazabicyclo[3.2.1]octan-8-yl)-N-(4,4-difluorocyclohexyl)-4,6-dihydro-5H-pyrrolo[3,4-d]thiazole-5-carboxamide;
2-(3,8-diazabicyclo[3.2.1]octan-8-yl)-N-(4,4-difluorocyclohexyl)-6,7-dihydropyrazolo[1,5-a]pyrazine-5(4H)-carboxamide;
1-(2-(3,8-diazabicyclo[3.2.1]octan-8-yl)-6,7-dihydropyrazolo[1,5-a]pyrazin-5(4H)-yl)-2-(4,4-difluorocyclohexyl)ethan-1-one;
1-(2-(3,8-diazabicyclo[3.2.1]octan-8-yl)-6,7-dihydropyrazolo[1,5-a]pyrazin-5(4H)-yl)-2-(4-fluorophenoxy)ethan-1-one;
1-(2-(3,8-diazabicyclo[3.2.1]octan-8-yl)-6,7-dihydrothiazolo[5,4-c]pyridin-5(4H)-yl)-2-((2-fluoropyridin-4-yl)oxy)ethan-1-one;
1-(2-(3,8-diazabicyclo[3.2.1]octan-8-yl)-6,7-dihydrothiazolo[5,4-c]pyridin-5(4H)-yl)-2-((6-fluoropyridin-3-yl)oxy)ethan-1-one;
2-cyclopentyl-1-(2-(3-methyl-3,8-diazabicyclo[3.2.1]octan-8-yl)-6,7-dihydrothiazolo[5,4-c]pyridin-5(4H)-yl)ethan-1-one;
2-(4,4-difluorocyclohexyl)-1-(2-(3-methyl-3,8-diazabicyclo[3.2.1]octan-8-yl)-6,7-dihydrothiazolo[5,4-c]pyridin-5(4H)-yl)ethan-1-one;
N-(4,4-difluorocyclohexyl)-2-(3-methyl-3,8-diazabicyclo[3.2.1]octan-8-yl)-6,7-dihydrothiazolo[5,4-c]pyridine-5(4H)-carboxamide;
N-cyclopentyl-2-(3-methyl-3,8-diazabicyclo[3.2.1]octan-8-yl)-6,7-dihydrothiazolo[5,4-c]pyridine-5(4H)-carboxamide;
2-((2-fluoropyridin-4-yl)oxy)-1-(2-(3-methyl-3,8-diazabicyclo[3.2.1]octan-8-yl)-6,7-dihydrothiazolo[5,4-c]pyridin-5(4H)-yl)ethan-1-one;
2-((6-fluoropyridin-3-yl)oxy)-1-(2-(3-methyl-3,8-diazabicyclo[3.2.1]octan-8-yl)-6,7-dihydrothiazolo[5,4-c]pyridin-5(4H)-yl)ethan-1-one;
N-(4,4-difluorocyclohexyl)-2-(3-methyl-3,8-diazabicyclo[3.2.1]octan-8-yl)-4,6-dihydro-5H-pyrrolo[3,4-d]thiazole-5-carboxamide;
N-cyclopentyl-2-(3-methyl-3,8-diazabicyclo[3.2.1]octan-8-yl)-4,6-dihydro-5H-pyrrolo[3,4-d]thiazole-5-carboxamide;
2-cyclopentyl-1-(2-(3-methyl-3,8-diazabicyclo[3.2.1]octan-8-yl)-4,6-dihydro-5H-pyrrolo[3,4-d]thiazol-5-yl)ethan-1-one;
2-(4,4-difluorocyclohexyl)-1-(2-(3-methyl-3,8-diazabicyclo[3.2.1]octan-8-yl)-4,6-dihydro-5H-pyrrolo[3,4-d]thiazol-5-yl)ethan-1-one;
N-(4,4-difluorocyclohexyl)-2-(3-methyl-3,8-diazabicyclo[3.2.1]octan-8-yl)-6,7-dihydropyrazolo[1,5-a]pyrazine-5(4H)-carboxamide;
2-(4,4-difluorocyclohexyl)-1-(2-(3-methyl-3,8-diazabicyclo[3.2.1]octan-8-yl)-6,7-dihydropyrazolo[1,5-a]pyrazin-5(4H)-yl)ethan-1-one;
1-(2-(3,8-diazabicyclo[3.2.1]octan-8-yl)-5,6-dihydro-[1,2,4]triazolo[1,5-a]pyrazin-7 (8H)-yl)-2-(4,4-difluorocyclohexyl)ethan-1-one;
1-(2-(3,8-diazabicyclo[3.2.1]octan-8-yl)-7,7-dimethyl-6,7-dihydrothiazolo[5,4-c]pyridin-5(4H)-yl)-2-(4,4-difluorocyclohexyl)ethan-1-one;
1-(2-(3,8-diazabicyclo[3.2.1]octan-8-yl)-7,7-dimethyl-6,7-dihydrothiazolo[5,4-c]pyridin-5(4H)-yl)-2-cyclopentylethan-1-one;
2-(4,4-difluorocyclohexyl)-1-(7,7-dimethyl-2-(piperazin-1-yl)-6,7-dihydrothiazolo[5,4-c]pyridin-5(4H)-yl)ethan-1-one;
2-cyclopentyl-1-(7,7-dimethyl-2-(piperazin-1-yl)-6,7-dihydrothiazolo[5,4-c]pyridin-5(4H)-yl)ethan-1-one;
2-cyclopentyl-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one;
2-(4,4-difluorocyclohexyl)-1-(7,7-dimethyl-2-(3-methyl-3,8-diazabicyclo[3.2.1]octan-8-yl)-6,7-dihydrothiazolo[5,4-c]pyridin-5(4H)-yl)ethan-1-one;
2-(4,4-difluorocyclohexyl)-1-(7,7-dimethyl-2-(4-methylpiperazin-1-yl)-6,7-dihydrothiazolo[5,4-c]pyridin-5(4H)-yl)ethan-1-one;
2-(3,8-diazabicyclo[3.2.1]octan-8-yl)-5-((cyclopentylmethyl) sulfonyl)-4,5,6,7-tetrahydrothiazolo[5,4-c]pyridine;
2-(3,8-diazabicyclo[3.2.1]octan-8-yl)-N-cyclopentyl-6,7-dihydrothiazolo[5,4-c]pyridine-5(4H)-sulfonamide;
2-cyclopentyl-1-(2-(piperazin-1-yl)-6,7-dihydrothiazolo[5,4-c]pyridin-5(4H)-yl)ethan-1-one;
2-(4-fluorophenoxy)-1-(2-(piperazin-1-yl)-6,7-dihydrothiazolo[5,4-c]pyridin-5(4H)-yl)ethan-1-one;
(S)-2-cyclopentyl-1-(2-(3-(hydroxymethyl) piperazin-1-yl)-6,7-dihydrothiazolo[5,4-c]pyridin-5(4H)-yl)ethan-1-one;
(R)-2-cyclopentyl-1-(2-(3-(hydroxymethyl) piperazin-1-yl)-6,7-dihydrothiazolo[5,4-c]pyridin-5(4H)-yl)ethan-1-one;
2-cyclopentyl-1-(2-(piperidin-4-yl)-6,7-dihydrothiazolo[5,4-c]pyridin-5(4H)-yl)ethan-1-one;
1-(2-(3,8-diazabicyclo[3.2.1]octan-8-yl)-6,7-dihydrothiazolo[5,4-c]pyridin-5(4H)-yl)-2-(3,3-dimethylcyclobutyl)ethan-1-one;
(2-(3,8-diazabicyclo[3.2.1]octan-8-yl)-6,7-dihydrothiazolo[5,4-c]pyridin-5(4H)-yl)(2,2-dimethylpyrrolidin-1-yl)methanone;
(2-(3,8-diazabicyclo[3.2.1]octan-8-yl)-6,7-dihydrothiazolo[5,4-c]pyridin-5(4H)-yl)(3,3-dimethylpyrrolidin-1-yl)methanone;
(2-(3,8-diazabicyclo[3.2.1]octan-8-yl)-6,7-dihydrothiazolo[5,4-c]pyridin-5(4H)-yl)(2-azaspiro[4.4]nonan-2-yl)methanone;
(2-(3,8-diazabicyclo[3.2.1]octan-8-yl)-6,7-dihydrothiazolo[5,4-c]pyridin-5(4H)-yl)(6-azaspiro[3.4]octan-6-yl)methanone;
2-(3,8-diazabicyclo[3.2.1]octan-8-yl)-N-(1-methylcyclobutyl)-6,7-dihydrothiazolo[5,4-c]pyridine-5(4H)-carboxamide;
2-(3,8-diazabicyclo[3.2.1]octan-8-yl)-N-(1-methylcyclopentyl)-6,7-dihydrothiazolo[5,4-c]pyridine-5(4H)-carboxamide;
1-(2-(3,8-diazabicyclo[3.2.1]octan-8-yl)-6,7-dihydropyrazolo[1,5-a]pyrazin-5(4H)-yl)-2,2-difluoro-2-phenylethan-1-one;
1-(2-(3,8-diazabicyclo[3.2.1]octan-8-yl)-6,7-dihydropyrazolo[1,5-a]pyrazin-5(4H)-yl)-2-cyclopentylethan-1-one;
1-(2-(3,8-diazabicyclo[3.2.1]octan-8-yl)-6,7-dihydropyrazolo[1,5-a]pyrazin-5(4H)-yl)-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-(2-(2-methoxyethoxy) phenoxy)ethan-1-one;
1-(2-(3,8-diazabicyclo[3.2.1]octan-8-yl)-6,7-dihydrothiazolo[5,4-c]pyridin-5(4H)-yl)-2-(1-methylcyclobutyl)ethan-1-one;
1-(2-(3,8-diazabicyclo[3.2.1]octan-8-yl)-6,7-dihydrothiazolo[5,4-c]pyridin-5(4H)-yl)-2-(4-fluorophenyl)-2-methoxyethan-1-one;
1-(2-(3,8-diazabicyclo[3.2.1]octan-8-yl)-6,7-dihydrothiazolo[5,4-c]pyridin-5(4H)-yl)-2-(4-fluorophenyl)-2-hydroxyethan-1-one;
(2-(3,8-diazabicyclo[3.2.1]octan-8-yl)-6,7-dihydrothiazolo[5,4-c]pyridin-5(4H)-yl)(tetrahydrofuran-2-yl)methanone;
1-(2-(3,8-diazabicyclo[3.2.1]octan-8-yl)-6,7-dihydrothiazolo[5,4-c]pyridin-5(4H)-yl)-2-(1-methoxycyclopentyl)ethan-1-one;
1-(2-(3,8-diazabicyclo[3.2.1]octan-8-yl)-6,7-dihydrothiazolo[5,4-c]pyridin-5(4H)-yl)-2-(1-(methoxymethyl)cyclopentyl)ethan-1-one;
1-(2-(3,8-diazabicyclo[3.2.1]octan-8-yl)-6,7-dihydrothiazolo[5,4-c]pyridin-5(4H)-yl)-2-(pyrazin-2-yl)ethan-1-one;
1-(2-(3,8-diazabicyclo[3.2.1]octan-8-yl)-6,7-dihydrothiazolo[5,4-c]pyridin-5(4H)-yl)-2-(3,3-difluorocyclopentyl)ethan-1-one;
1-(2-(3,8-diazabicyclo[3.2.1]octan-8-yl)-6,7-dihydrothiazolo[5,4-c]pyridin-5(4H)-yl)-2-(2-methoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2-methoxyphenyl)ethan-1-one;
1-(2-(3,8-diazabicyclo[3.2.1]octan-8-yl)-6,7-dihydrothiazolo[5,4-c]pyridin-5(4H)-yl)-2-(4-fluoro-2-methoxyphenyl)ethan-1-one;
1-(2-(3,8-diazabicyclo[3.2.1]octan-8-yl)-6,7-dihydrothiazolo[5,4-c]pyridin-5(4H)-yl)-2-(2-fluoropyridin-4-yl)ethan-1-one;
1-(2-(3,8-diazabicyclo[3.2.1]octan-8-yl)-6,7-dihydrothiazolo[5,4-c]pyridin-5(4H)-yl)-3-(6-fluoropyridin-3-yl)propan-1-one;
1-(2-(3,8-diazabicyclo[3.2.1]octan-8-yl)-6,7-dihydrothiazolo[5,4-c]pyridin-5(4H)-yl)-2-(4-fluoro-2-methoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2,4-difluorophenyl)ethan-1-one;
1-(2-(3,8-diazabicyclo[3.2.1]octan-8-yl)-6,7-dihydrothiazolo[5,4-c]pyridin-5(4H)-yl)-2-(4-fluoro-2-methylphenoxy)ethan-1-one;
1-(2-(3,8-diazabicyclo[3.2.1]octan-8-yl)-6,7-dihydrothiazolo[5,4-c]pyridin-5(4H)-yl)-2-cyclopentyl-2-methylpropan-1-one;
1-(2-(3,8-diazabicyclo[3.2.1]octan-8-yl)-6,7-dihydrothiazolo[5,4-c]pyridin-5(4H)-yl)-3-(4-fluoro-2-methoxyphenyl)propan-1-one;
1-(2-(3,8-diazabicyclo[3.2.1]octan-8-yl)-6,7-dihydrothiazolo[5,4-c]pyridin-5(4H)-yl)-2-(2,4-difluorophenoxy)ethan-1-one;
1-(2-(3,8-diazabicyclo[3.2.1]octan-8-yl)-6,7-dihydrothiazolo[5,4-c]pyridin-5(4H)-yl)-2-(cyclopentyloxy)ethan-1-one;
(2-(3,8-diazabicyclo[3.2.1]octan-8-yl)-6,7-dihydrothiazolo[5,4-c]pyridin-5(4H)-yl)(1-methylcyclopentyl)methanone;
(2-(3,8-diazabicyclo[3.2.1]octan-8-yl)-6,7-dihydrothiazolo[5,4-c]pyridin-5(4H)-yl)(1-fluorocyclopentyl)methanone;
3-(2-(3,8-diazabicyclo[3.2.1]octan-8-yl)-6,7-dihydrothiazolo[5,4-c]pyridin-5(4H)-yl)-2-cyclopentyl-3-oxopropanenitrile;
1-(2-(3,8-diazabicyclo[3.2.1]octan-8-yl)-6,7-dihydrothiazolo[5,4-c]pyridin-5(4H)-yl)-2-(2,6-dimethylpyridin-4-yl)ethan-1-one;
1-(2-(3,8-diazabicyclo[3.2.1]octan-8-yl)-6,7-dihydrothiazolo[5,4-c]pyridin-5(4H)-yl)-2,2-dimethylbutan-1-one;
1-(2-(3,8-diazabicyclo[3.2.1]octan-8-yl)-6,7-dihydrothiazolo[5,4-c]pyridin-5(4H)-yl)-2-(2-oxaspiro[3.5]nonan-7-yl)ethan-1-one;
1-(2-(3,8-diazabicyclo[3.2.1]octan-8-yl)-6,7-dihydrothiazolo[5,4-c]pyridin-5(4H)-yl)-2-(4-fluoro-2-methylphenyl)ethan-1-one;
1-(2-(3,8-diazabicyclo[3.2.1]octan-8-yl)-6,7-dihydrothiazolo[5,4-c]pyridin-5(4H)-yl)-2-cyclopentyl-2-methoxyethan-1-one;
1-(2-(3,8-diazabicyclo[3.2.1]octan-8-yl)-6,7-dihydrothiazolo[5,4-c]pyridin-5(4H)-yl)-2,3,3-trimethylbutan-1-one (enantiomer-1);
1-(2-(3,8-diazabicyclo[3.2.1]octan-8-yl)-6,7-dihydrothiazolo[5,4-c]pyridin-5(4H)-yl)-2,3,3-trimethylbutan-1-one (enantiomer-2);
1-(2-(3,8-diazabicyclo[3.2.1]octan-8-yl)-6,7-dihydrothiazolo[5,4-c]pyridin-5(4H)-yl)-2-(bicyclo[2.2.1]heptan-1-yl)ethan-1-one;
1-(2-(3,8-diazabicyclo[3.2.1]octan-8-yl)-6,7-dihydrothiazolo[5,4-c]pyridin-5(4H)-yl)-2,2,3-trimethylbutan-1-one;
(2-(3,8-diazabicyclo[3.2.1]octan-8-yl)-6,7-dihydrothiazolo[5,4-c]pyridin-5(4H)-yl)(spiro[2.4]heptan-1-yl)methanone;
1-(2-(3,8-diazabicyclo[3.2.1]octan-8-yl)-6,7-dihydrothiazolo[5,4-c]pyridin-5(4H)-yl)-2-(2-isopropoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2-(trifluoromethoxy) phenoxy)ethan-1-one;
1-(2-(3,8-diazabicyclo[3.2.1]octan-8-yl)-6,7-dihydrothiazolo[5,4-c]pyridin-5(4H)-yl)-2-(6-fluoropyridin-3-yl)ethan-1-one;
1-(2-(3,8-diazabicyclo[3.2.1]octan-8-yl)-6,7-dihydrothiazolo[5,4-c]pyridin-5(4H)-yl)-2,2-difluoro-2-(6-fluoropyridin-3-yl)ethan-1-one;
2-(2-(2-(3,8-diazabicyclo[3.2.1]octan-8-yl)-6,7-dihydrothiazolo[5,4-c]pyridin-5(4H)-yl)-2-oxoethoxy)benzonitrile;
2-(2-(2-(3,8-diazabicyclo[3.2.1]octan-8-yl)-6,7-dihydrothiazolo[5,4-c]pyridin-5(4H)-yl)-2-oxoethoxy)-3-methoxybenzonitrile;
1-(2-(3,8-diazabicyclo[3.2.1]octan-8-yl)-6,7-dihydrothiazolo[5,4-c]pyridin-5(4H)-yl)-2-(cyclopentyloxy)propan-1-one;
(2S)-1-(2-(3,8-diazabicyclo[3.2.1]octan-8-yl)-6,7-dihydrothiazolo[5,4-c]pyridin-5(4H)-yl)-2-cyclopentyl-2-hydroxyethan-1-one;
(2R)-1-(2-(3,8-diazabicyclo[3.2.1]octan-8-yl)-6,7-dihydrothiazolo[5,4-c]pyridin-5(4H)-yl)-2-cyclopentyl-2-hydroxyethan-1-one;
1-(2-(3,8-diazabicyclo[3.2.1]octan-8-yl)-6,7-dihydrothiazolo[5,4-c]pyridin-5(4H)-yl)-2-(2,6-dimethoxyphenoxy)ethan-1-one;
1-(2-(3,8-diazabicyclo[3.2.1]octan-8-yl)-6,7-dihydrothiazolo[5,4-c]pyridin-5(4H)-yl)-2-(2-(difluoromethyl) phenoxy)ethan-1-one;
1-(2-(3,8-diazabicyclo[3.2.1]octan-8-yl)-6,7-dihydrothiazolo[5,4-c]pyridin-5(4H)-yl)-3,3-dimethylpentan-1-one;
1-(2-(3,8-diazabicyclo[3.2.1]octan-8-yl)-6,7-dihydrothiazolo[5,4-c]pyridin-5(4H)-yl)-4,4-difluoro-3,3-dimethylbutan-1-one;
(2R)-1-(2-(3,8-diazabicyclo[3.2.1]octan-8-yl)-6,7-dihydrothiazolo[5,4-c]pyridin-5(4H)-yl)-2-hydroxy-3,3-dimethylbutan-1-one;
(2R)-1-(2-(3,8-diazabicyclo[3.2.1]octan-8-yl)-6,7-dihydrothiazolo[5,4-c]pyridin-5(4H)-yl)-2-cyclohexyl-2-hydroxyethan-1-one;
4-(2-(3,8-diazabicyclo[3.2.1]octan-8-yl)-6,7-dihydrothiazolo[5,4-c]pyridin-5(4H)-yl)-2,2-dimethyl-4-oxobutanenitrile;
(2-(3,8-diazabicyclo[3.2.1]octan-8-yl)-6,7-dihydrothiazolo[5,4-c]pyridin-5(4H)-yl)(2,3-dihydrobenzofuran-2-yl)methanone;
(2-(3,8-diazabicyclo[3.2.1]octan-8-yl)-6,7-dihydrothiazolo[5,4-c]pyridin-5(4H)-yl)(2,3-dihydrobenzo[b][1,4]dioxin-2-yl)methanone;
(2-(3,8-diazabicyclo[3.2.1]octan-8-yl)-6,7-dihydrothiazolo[5,4-c]pyridin-5(4H)-yl)(chroman-2-yl)methanone;
(2-(3,8-diazabicyclo[3.2.1]octan-8-yl)-6,7-dihydrothiazolo[5,4-c]pyridin-5(4H)-yl)(chroman-3-yl)methanone;
(2-(3,8-diazabicyclo[3.2.1]octan-8-yl)-6,7-dihydrothiazolo[5,4-c]pyridin-5(4H)-yl)(isochroman-3-yl)methanone;
(2-(3,8-diazabicyclo[3.2.1]octan-8-yl)-6,7-dihydrothiazolo[5,4-c]pyridin-5(4H)-yl)(2,3-dihydrobenzofuran-3-yl)methanone;
(2-(3,8-diazabicyclo[3.2.1]octan-8-yl)-6,7-dihydrothiazolo[5,4-c]pyridin-5(4H)-yl)(benzofuran-2-yl)methanone;
(2-(3,8-diazabicyclo[3.2.1]octan-8-yl)-6,7-dihydrothiazolo[5,4-c]pyridin-5(4H)-yl)(isoquinolin-1-yl)methanone;
(2-(3,8-diazabicyclo[3.2.1]octan-8-yl)-6,7-dihydrothiazolo[5,4-c]pyridin-5(4H)-yl)(benzofuran-3-yl)methanone;
(2-(3,8-diazabicyclo[3.2.1]octan-8-yl)-6,7-dihydrothiazolo[5,4-c]pyridin-5(4H)-yl)(1H-indol-2-yl)methanone;
(2-(3,8-diazabicyclo[3.2.1]octan-8-yl)-6,7-dihydrothiazolo[5,4-c]pyridin-5(4H)-yl)(1-methyl-1H-indol-2-yl)methanone;
(2-(3,8-diazabicyclo[3.2.1]octan-8-yl)-6,7-dihydrothiazolo[5,4-c]pyridin-5(4H)-yl)(benzo[d]oxazol-2-yl)methanone;
(2-(3,8-diazabicyclo[3.2.1]octan-8-yl)-6,7-dihydrothiazolo[5,4-c]pyridin-5(4H)-yl)(isoquinolin-3-yl)methanone;
(2-(3,8-diazabicyclo[3.2.1]octan-8-yl)-6,7-dihydrothiazolo[5,4-c]pyridin-5(4H)-yl)(phenyl)methanone;
(2-(3,8-diazabicyclo[3.2.1]octan-8-yl)-6,7-dihydrothiazolo[5,4-c]pyridin-5(4H)-yl)(o-tolyl)methanone;
(2-(3,8-diazabicyclo[3.2.1]octan-8-yl)-6,7-dihydrothiazolo[5,4-c]pyridin-5(4H)-yl)(m-tolyl)methanone;
(2-(3,8-diazabicyclo[3.2.1]octan-8-yl)-6,7-dihydrothiazolo[5,4-c]pyridin-5(4H)-yl)(p-tolyl)methanone;
(2-(3,8-diazabicyclo[3.2.1]octan-8-yl)-6,7-dihydrothiazolo[5,4-c]pyridin-5(4H)-yl)(2-methoxyphenyl)methanone;
(2-(3,8-diazabicyclo[3.2.1]octan-8-yl)-6,7-dihydrothiazolo[5,4-c]pyridin-5(4H)-yl)(4-fluorophenyl)methanone;
(2-(3,8-diazabicyclo[3.2.1]octan-8-yl)-6,7-dihydrothiazolo[5,4-c]pyridin-5(4H)-yl)(3-(difluoromethyl)phenyl)methanone;
(2-(3,8-diazabicyclo[3.2.1]octan-8-yl)-6,7-dihydrothiazolo[5,4-c]pyridin-5(4H)-yl)(6-fluoropyridin-3-yl)methanone;
(2-(3,8-diazabicyclo[3.2.1]octan-8-yl)-6,7-dihydrothiazolo[5,4-c]pyridin-5(4H)-yl)(pyridin-2-yl)methanone;
(2-(3,8-diazabicyclo[3.2.1]octan-8-yl)-6,7-dihydrothiazolo[5,4-c]pyridin-5(4H)-yl)(5-fluoropyridin-2-yl)methanone;
(2-(3,8-diazabicyclo[3.2.1]octan-8-yl)-6,7-dihydrothiazolo[5,4-c]pyridin-5(4H)-yl)(2-(difluoromethoxy)phenyl)methanone;
(2-(3,8-diazabicyclo[3.2.1]octan-8-yl)-6,7-dihydrothiazolo[5,4-c]pyridin-5(4H)-yl)(4-(difluoromethoxy)phenyl)methanone;
(2-(3,8-diazabicyclo[3.2.1]octan-8-yl)-6,7-dihydrothiazolo[5,4-c]pyridin-5(4H)-yl)(3-(trifluoromethoxy)phenyl)methanone;
(2-(3,8-diazabicyclo[3.2.1]octan-8-yl)-6,7-dihydrothiazolo[5,4-c]pyridin-5(4H)-yl)(3-(difluoromethoxy)phenyl)methanone;
(2-(3,8-diazabicyclo[3.2.1]octan-8-yl)-6,7-dihydrothiazolo[5,4-c]pyridin-5(4H)-yl)(2-(trifluoromethoxy)phenyl)methanone;
(2-(3,8-diazabicyclo[3.2.1]octan-8-yl)-6,7-dihydrothiazolo[5,4-c]pyridin-5(4H)-yl)(4-(trifluoromethoxy)phenyl)methanone;
(2-(3,8-diazabicyclo[3.2.1]octan-8-yl)-6,7-dihydrothiazolo[5,4-c]pyridin-5(4H)-yl)(2-fluorophenyl)methanone;
(2-(3,8-diazabicyclo[3.2.1]octan-8-yl)-6,7-dihydrothiazolo[5,4-c]pyridin-5(4H)-yl)(2-chlorophenyl)methanone;
(2-(3,8-diazabicyclo[3.2.1]octan-8-yl)-6,7-dihydrothiazolo[5,4-c]pyridin-5(4H)-yl)(2-isopropoxyphenyl)methanone;
(2-(3,8-diazabicyclo[3.2.1]octan-8-yl)-6,7-dihydrothiazolo[5,4-c]pyridin-5(4H)-yl)(2,4-difluorophenyl)methanone;
2-(2-(3,8-diazabicyclo[3.2.1]octan-8-yl)-4,5,6,7-tetrahydrothiazolo[5,4-c]pyridine-5-carbonyl)benzonitrile;
(2-(3,8-diazabicyclo[3.2.1]octan-8-yl)-6,7-dihydrothiazolo[5,4-c]pyridin-5(4H)-yl)(2-(methoxymethyl)phenyl)methanone;
(2-(3,8-diazabicyclo[3.2.1]octan-8-yl)-6,7-dihydrothiazolo[5,4-c]pyridin-5(4H)-yl)(2-isopropylphenyl)methanone;
(2-(3,8-diazabicyclo[3.2.1]octan-8-yl)-6,7-dihydrothiazolo[5,4-c]pyridin-5(4H)-yl)(4-fluoro-2-methylphenyl)methanone;
(2-(3,8-diazabicyclo[3.2.1]octan-8-yl)-6,7-dihydrothiazolo[5,4-c]pyridin-5(4H)-yl)(chroman-8-yl)methanone;
(2-(3,8-diazabicyclo[3.2.1]octan-8-yl)-6,7-dihydrothiazolo[5,4-c]pyridin-5(4H)-yl)(2-(difluoromethyl)phenyl)methanone;
(2-(3,8-diazabicyclo[3.2.1]octan-8-yl)-6,7-dihydrothiazolo[5,4-c]pyridin-5(4H)-yl)(2-methoxy-4-methylphenyl)methanone;
(2-(3,8-diazabicyclo[3.2.1]octan-8-yl)-6,7-dihydrothiazolo[5,4-c]pyridin-5(4H)-yl)(2-cyclopropylphenyl)methanone;
(2-(3,8-diazabicyclo[3.2.1]octan-8-yl)-6,7-dihydrothiazolo[5,4-c]pyridin-5(4H)-yl)(6-chloropyridin-3-yl)methanone;
(2-(3,8-diazabicyclo[3.2.1]octan-8-yl)-6,7-dihydrothiazolo[5,4-c]pyridin-5(4H)-yl)(4-fluoro-2-methoxyphenyl)methanone;
(2-(3,8-diazabicyclo[3.2.1]octan-8-yl)-6,7-dihydrothiazolo[5,4-c]pyridin-5(4H)-yl)(2-(1,1-difluoroethyl)phenyl)methanone;
(2-(3,8-diazabicyclo[3.2.1]octan-8-yl)-6,7-dihydrothiazolo[5,4-c]pyridin-5(4H)-yl)(2-chloropyridin-3-yl)methanone;
(2-(3,8-diazabicyclo[3.2.1]octan-8-yl)-6,7-dihydrothiazolo[5,4-c]pyridin-5(4H)-yl)(2,4-dimethylphenyl)methanone;
(2-(3,8-diazabicyclo[3.2.1]octan-8-yl)-6,7-dihydrothiazolo[5,4-c]pyridin-5(4H)-yl)(4-chlorophenyl)methanone;
(2-(3,8-diazabicyclo[3.2.1]octan-8-yl)-6,7-dihydrothiazolo[5,4-c]pyridin-5(4H)-yl)(4-(trifluoromethyl)phenyl)methanone;
(2-(3,8-diazabicyclo[3.2.1]octan-8-yl)-6,7-dihydrothiazolo[5,4-c]pyridin-5(4H)-yl)(4-cyclopropylphenyl)methanone;
(2-(3,8-diazabicyclo[3.2.1]octan-8-yl)-6,7-dihydrothiazolo[5,4-c]pyridin-5(4H)-yl)(4-(difluoromethyl)phenyl)methanone;
(2-(3,8-diazabicyclo[3.2.1]octan-8-yl)-6,7-dihydrothiazolo[5,4-c]pyridin-5(4H)-yl)(2,3-dihydrobenzofuran-7-yl)methanone;
(2-(3,8-diazabicyclo[3.2.1]octan-8-yl)-6,7-dihydrothiazolo[5,4-c]pyridin-5(4H)-yl)(4-(2,2-difluoroethyl)phenyl)methanone;
(2-(3,8-diazabicyclo[3.2.1]octan-8-yl)-6,7-dihydrothiazolo[5,4-c]pyridin-5(4H)-yl)(4-(2,2,2-trifluoroethyl)phenyl)methanone;
(2-(3,8-diazabicyclo[3.2.1]octan-8-yl)-6,7-dihydrothiazolo[5,4-c]pyridin-5(4H)-yl)(2,2-difluorobenzo[d][1,3]dioxol-4-yl)methanone;
(2-(3,8-diazabicyclo[3.2.1]octan-8-yl)-6,7-dihydrothiazolo[5,4-c]pyridin-5(4H)-yl)(benzo[d][1,3]dioxol-4-yl)methanone;
(2-(3,8-diazabicyclo[3.2.1]octan-8-yl)-6,7-dihydrothiazolo[5,4-c]pyridin-5(4H)-yl)(3-fluorophenyl)methanone;
(2-(3,8-diazabicyclo[3.2.1]octan-8-yl)-6,7-dihydrothiazolo[5,4-c]pyridin-5(4H)-yl)(2,3-dihydrobenzofuran-4-yl)methanone;
(2-(3,8-diazabicyclo[3.2.1]octan-8-yl)-6,7-dihydrothiazolo[5,4-c]pyridin-5(4H)-yl)(quinolin-8-yl)methanone;
2-(2-(3,8-diazabicyclo[3.2.1]octan-8-yl)-4,5,6,7-tetrahydrothiazolo[5,4-c]pyridine-5-carbonyl)-5-methylbenzonitrile;
4-(2-(3,8-diazabicyclo[3.2.1]octan-8-yl)-4,5,6,7-tetrahydrothiazolo[5,4-c]pyridine-5-carbonyl)benzonitrile;
(2-(3,8-diazabicyclo[3.2.1]octan-8-yl)-6,7-dihydrothiazolo[5,4-c]pyridin-5(4H)-yl)(6-fluoro-2-methoxypyridin-3-yl)methanone;
(2-(3,8-diazabicyclo[3.2.1]octan-8-yl)-6,7-dihydrothiazolo[5,4-c]pyridin-5(4H)-yl)(6-(trifluoromethyl)pyridin-3-yl)methanone;
(2-(3,8-diazabicyclo[3.2.1]octan-8-yl)-6,7-dihydrothiazolo[5,4-c]pyridin-5(4H)-yl)(6-fluoro-4-methylpyridin-3-yl)methanone;
(2-(3,8-diazabicyclo[3.2.1]octan-8-yl)-6,7-dihydrothiazolo[5,4-c]pyridin-5(4H)-yl)(6-(difluoromethyl)pyridin-3-yl)methanone;
cyclopentyl 2-(3,8-diazabicyclo[3.2.1]octan-8-yl)-6,7-dihydrothiazolo[5,4-c]pyridine-5(4H)-carboxylate;
isopropyl 2-(3,8-diazabicyclo[3.2.1]octan-8-yl)-6,7-dihydrothiazolo[5,4-c]pyridine-5(4H)-carboxylate;
benzyl 2-(3,8-diazabicyclo[3.2.1]octan-8-yl)-6,7-dihydrothiazolo[5,4-c]pyridine-5(4H)-carboxylate;
phenyl 2-(3,8-diazabicyclo[3.2.1]octan-8-yl)-6,7-dihydrothiazolo[5,4-c]pyridine-5(4H)-carboxylate;
1-(2-(3,8-diazabicyclo[3.2.1]octan-8-yl)-6,7-dihydrothiazolo[5,4-c]pyridin-5(4H)-yl)-2-hydroxy-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-methoxy-2-(1-methylcyclopentyl)ethan-1-one;
1-(2-(3,8-diazabicyclo[3.2.1]octan-8-yl)-6,7-dihydrothiazolo[5,4-c]pyridin-5(4H)-yl)-2-(2-(2-hydroxyethoxy) phenoxy)ethan-1-one;
1-(2-(3,8-diazabicyclo[3.2.1]octan-8-yl)-6,7-dihydrothiazolo[5,4-c]pyridin-5(4H)-yl)-2,2-difluoro-2-(2-fluoropyridin-4-yl)ethan-1-one;
1-(2-(3,8-diazabicyclo[3.2.1]octan-8-yl)-6,7-dihydrothiazolo[5,4-c]pyridin-5(4H)-yl)-2-(2-(difluoromethoxy) phenoxy)ethan-1-one;
2-(2-(2-(3,8-diazabicyclo[3.2.1]octan-8-yl)-6,7-dihydrothiazolo[5,4-c]pyridin-5(4H)-yl)-2-oxoethoxy)-5-fluoro-3-methoxybenzonitrile;
1-(2-(3,8-diazabicyclo[3.2.1]octan-8-yl)-6,7-dihydrothiazolo[5,4-c]pyridin-5(4H)-yl)-2-cyclopentyl-2-hydroxypropan-1-one (enantiomer-1);
1-(2-(3,8-diazabicyclo[3.2.1]octan-8-yl)-6,7-dihydrothiazolo[5,4-c]pyridin-5(4H)-yl)-2-cyclopentyl-2-hydroxypropan-1-one (enantiomer-2);
(2-(3,8-diazabicyclo[3.2.1]octan-8-yl)-7,7-dimethyl-6,7-dihydrothiazolo[5,4-c]pyridin-5(4H)-yl)(cyclopentyl)methanone;
1-(2-(3,8-diazabicyclo[3.2.1]octan-8-yl)-7,7-dimethyl-6,7-dihydrothiazolo[5,4-c]pyridin-5(4H)-yl)-3-methylbutan-1-one;
1-(2-(3,8-diazabicyclo[3.2.1]octan-8-yl)-7,7-dimethyl-6,7-dihydrothiazolo[5,4-c]pyridin-5(4H)-yl)-2-methylpropan-1-one;
1-(2-(3,8-diazabicyclo[3.2.1]octan-8-yl)-7,7-dimethyl-6,7-dihydrothiazolo[5,4-c]pyridin-5(4H)-yl)-2-hydroxy-3-methylbutan-1-one;
1-(2-(3,8-diazabicyclo[3.2.1]octan-8-yl)-7,7-dimethyl-6,7-dihydrothiazolo[5,4-c]pyridin-5(4H)-yl)-2-(2-oxaspiro[3.5]nonan-7-yl)ethan-1-one;
3-(2-(3,8-diazabicyclo[3.2.1]octan-8-yl)-7,7-dimethyl-6,7-dihydrothiazolo[5,4-c]pyridin-5(4H)-yl)-2-cyclopentyl-3-oxopropanenitrile;
(2R)-1-(2-(3,8-diazabicyclo[3.2.1]octan-8-yl)-7,7-dimethyl-6,7-dihydrothiazolo[5,4-c]pyridin-5(4H)-yl)-2-cyclopentyl-2-hydroxyethan-1-one;
(2-(3,8-diazabicyclo[3.2.1]octan-8-yl)-7,7-dimethyl-6,7-dihydrothiazolo[5,4-c]pyridin-5(4H)-yl)(phenyl)methanone;
(2-(3,8-diazabicyclo[3.2.1]octan-8-yl)-7,7-dimethyl-6,7-dihydrothiazolo[5,4-c]pyridin-5(4H)-yl)(6-fluoropyridin-3-yl)methanone;
1-(2′-(3,8-diazabicyclo[3.2.1]octan-8-yl)-4′H-spiro[cyclopropane-1,7′-thiazolo[5,4-c]pyridin]-5′ (6′H)-yl)-2-cyclopentylethan-1-one;
1-(2′-(3,8-diazabicyclo[3.2.1]octan-8-yl)-2,3,5,6-tetrahydro-4′H-spiro[pyran-4,7′-thiazolo[5,4-c]pyridin]-5′ (6′H)-yl)-2-cyclopentylethan-1-one;
1-(2-(3,8-diazabicyclo[3.2.1]octan-8-yl)-6,6-dimethyl-6,7-dihydrothiazolo[5,4-c]pyridin-5(4H)-yl)-2-cyclopentylethan-1-one;
1-(2-(3,8-diazabicyclo[3.2.1]octan-8-yl)-6,6-dimethyl-6,7-dihydrothiazolo[5,4-c]pyridin-5(4H)-yl)-2-methylpropan-1-one;
(2-(3,8-diazabicyclo[3.2.1]octan-8-yl)-6,6-dimethyl-6,7-dihydrothiazolo[5,4-c]pyridin-5(4H)-yl)(phenyl)methanone;
1-(2-(3,8-diazabicyclo[3.2.1]octan-8-yl)-7,7-difluoro-6,7-dihydrothiazolo[5,4-c]pyridin-5(4H)-yl)-2-cyclopentylethan-1-one;
(S)-2-cyclopentyl-1-(2-(hexahydropyrrolo[1,2-a]pyrazin-2 (1H)-yl)-6,7-dihydrothiazolo[5,4-c]pyridin-5(4H)-yl)ethan-1-one;
(R)-2-cyclopentyl-1-(2-(hexahydropyrrolo[1,2-a]pyrazin-2 (1H)-yl)-6,7-dihydrothiazolo[5,4-c]pyridin-5(4H)-yl)ethan-1-one;
1-(2-(3,9-diazabicyclo[3.3.1]nonan-9-yl)-6,7-dihydrothiazolo[5,4-c]pyridin-5(4H)-yl)-2-cyclopentylethan-1-one;
3,3-dimethyl-1-(2-(piperazin-1-yl)-6,7-dihydrothiazolo[5,4-c]pyridin-5(4H)-yl) butan-1-one;
(R)-2-cyclopentyl-1-(2-(2-(hydroxymethyl) piperazin-1-yl)-6,7-dihydrothiazolo[5,4-c]pyridin-5(4H)-yl)ethan-1-one;
1-(2-(3,8-diazabicyclo[3.2.1]octan-3-yl)-6,7-dihydrothiazolo[5,4-c]pyridin-5(4H)-yl)-2-cyclopentylethan-1-one;
1-(2-((1 S,4S)-2,5-diazabicyclo[2.2.1]heptan-2-yl)-6,7-dihydrothiazolo[5,4-c]pyridin-5(4H)-yl)-2-cyclopentylethan-1-one;
1-(2-(2,5-diazabicyclo[2.2.2]octan-2-yl)-6,7-dihydrothiazolo[5,4-c]pyridin-5(4H)-yl)-2-cyclopentylethan-1-one;
1-(2-((1R,4R)-2,5-diazabicyclo[2.2.1]heptan-2-yl)-6,7-dihydrothiazolo[5,4-c]pyridin-5(4H)-yl)-2-cyclopentylethan-1-one;
1-(2-(3,9-diazabicyclo[3.3.1]nonan-3-yl)-6,7-dihydrothiazolo[5,4-c]pyridin-5(4H)-yl)-2-cyclopentylethan-1-one;
1-(2-(3,8-diazabicyclo[3.2.1]octan-8-yl)-6,7-dihydrothiazolo[4,5-c]pyridin-5(4H)-yl)-2-cyclopentylethan-1-one;
1-(2-(3,8-diazabicyclo[3.2.1]octan-8-yl)-6,7-dihydrothiazolo[4,5-c]pyridin-5(4H)-yl)-3,3-dimethylbutan-1-one;
2-cyclopentyl-1-(2-(7-hydroxy-3,9-diazabicyclo[3.3.1]nonan-9-yl)-6,7-dihydrothiazolo[5,4-c]pyridin-5(4H)-yl)ethan-1-one;
(S)-2-cyclopentyl-1-(2-(2-(hydroxymethyl) piperazin-1-yl)-6,7-dihydrothiazolo[5,4-c]pyridin-5(4H)-yl)ethan-1-one;
2-(3,8-diazabicyclo[3.2.1]octan-8-yl)-5-((4-fluorobenzyl)sulfonyl)-4,5,6,7-tetrahydrothiazolo[5,4-c]pyridine;
2-(3-ethyl-3,8-diazabicyclo[3.2.1]octan-8-yl)-5-((4-fluorobenzyl)sulfonyl)-4,5,6,7-tetrahydrothiazolo[5,4-c]pyridine;
5-((4-fluorobenzyl)sulfonyl)-2-(piperazin-1-yl)-4,5,6,7-tetrahydrothiazolo[5,4-c]pyridine;
5-(benzylsulfonyl)-2-(3,8-diazabicyclo[3.2.1]octan-8-yl)-4,5,6,7-tetrahydrothiazolo[5,4-c]pyridine;
5-(benzylsulfonyl)-2-(3-ethyl-3,8-diazabicyclo[3.2.1]octan-8-yl)-4,5,6,7-tetrahydrothiazolo[5,4-c]pyridine;
2-cyclopentyl-1-(2-(3-isopropyl-3,8-diazabicyclo[3.2.1]octan-8-yl)-6,7-dihydrothiazolo[5,4-c]pyridin-5(4H)-yl)ethan-1-one;
2-cyclopentyl-1-(2-(3-ethyl-3,8-diazabicyclo[3.2.1]octan-8-yl)-6,7-dihydrothiazolo[5,4-c]pyridin-5(4H)-yl)ethan-1-one;
2-cyclopentyl-1-(2-(3-propyl-3,8-diazabicyclo[3.2.1]octan-8-yl)-6,7-dihydrothiazolo[5,4-c]pyridin-5(4H)-yl)ethan-1-one;
2-(3,8-diazabicyclo[3.2.1]octan-8-yl)-N—((S)-chroman-4-yl)-6,7-dihydrothiazolo[5,4-c]pyridine-5(4H)-carboxamide;
N—((S)-chroman-4-yl)-2-(3-ethyl-3,8-diazabicyclo[3.2.1]octan-8-yl)-6,7-dihydrothiazolo[5,4-c]pyridine-5(4H)-carboxamide;
N—((R)-chroman-4-yl)-2-(3-ethyl-3,8-diazabicyclo[3.2.1]octan-8-yl)-6,7-dihydrothiazolo[5,4-c]pyridine-5(4H)-carboxamide;
(S)—N-(chroman-4-yl)-2-(piperazin-1-yl)-6,7-dihydrothiazolo[5,4-c]pyridine-5(4H)-carboxamide;
(S)—N-(chroman-4-yl)-2-(4-ethylpiperazin-1-yl)-6,7-dihydrothiazolo[5,4-c]pyridine-5(4H)-carboxamide;
2-(4-fluorophenoxy)-1-(2-(3-isopropyl-3,8-diazabicyclo[3.2.1]octan-8-yl)-6,7-dihydrothiazolo[5,4-c]pyridin-5(4H)-yl)ethan-1-one;
1-(2-(4-ethylpiperazin-1-yl)-6,7-dihydrothiazolo[5,4-c]pyridin-5(4H)-yl)-3,3-dimethylbutan-1-one;
2-(3-ethyl-3,8-diazabicyclo[3.2.1]octan-8-yl)-N-(4-fluorobenzyl)-6,7-dihydrothiazolo[5,4-c]pyridine-5(4H)-carboxamide;
2-(4-fluorophenoxy)-1-(2-(3-methyl-3,8-diazabicyclo[3.2.1]octan-8-yl)-6,7-dihydrothiazolo[5,4-c]pyridin-5(4H)-yl)ethan-1-one;
1-(2-(3-ethyl-3,8-diazabicyclo[3.2.1]octan-8-yl)-6,7-dihydrothiazolo[5,4-c]pyridin-5(4H)-yl)-2-(4-fluorophenoxy)ethan-1-one;
2-(4-fluorophenoxy)-1-(2-(3-propyl-3,8-diazabicyclo[3.2.1]octan-8-yl)-6,7-dihydrothiazolo[5,4-c]pyridin-5(4H)-yl)ethan-1-one;
2-(4-ethylpiperazin-1-yl)-5-((4-fluorobenzyl)sulfonyl)-4,5,6,7-tetrahydrothiazolo[5,4-c]pyridine;
or a pharmaceutically acceptable salt or derivative thereof.

35. A pharmaceutical composition comprising a compound or a pharmaceutically acceptable salt or derivative as defined in any of claims 1-34, and a pharmaceutically acceptable excipient.

36. A compound or pharmaceutically acceptable salt or derivative of any of claims 17-34 for use in therapy.

37. A compound or pharmaceutically acceptable salt or derivative of any of claims 17-34 or a pharmaceutical composition of claim 36 for use in the treatment or prevention of a disease or disorder that can be ameliorated by activation of long isoforms of PDE4 or a disease or disorder mediated by excessive intracellular cyclic AMP signalling.

38. The compound or pharmaceutically acceptable salt or derivative for use of any of claims 1 to 16 or the compound or pharmaceutically acceptable salt or derivative or pharmaceutical composition for use of claim 37 in the treatment or prevention of a disease or disorder mediated by excessive intracellular cyclic AMP signalling.

39. A method of treating or preventing a disease or disorder that can be ameliorated by activation of long isoforms of PDE4 or a disease or disorder mediated by excessive intracellular cyclic AMP signalling, comprising administering to a patient in need thereof a therapeutically effective amount of a compound or a pharmaceutically acceptable salt or derivative as defined in any of claims 1 to 34.

40. Use of a compound or a pharmaceutically acceptable salt or derivative as defined in any of claims 1 to 34, in the manufacture of a medicament for treatment or prevention of a disease or disorder that can be ameliorated by activation of long isoforms of PDE4 or a disease or disorder mediated by excessive intracellular cyclic AMP signalling.

41. The method of claim 39 or the use of claim 40, wherein disease or disorder that can be ameliorated by activation of long isoforms of PDE4 is a disease or disorder mediated by excessive intracellular cyclic AMP signalling.

42. The compound or pharmaceutically acceptable salt or derivative or pharmaceutical composition for use of claim 38, or the method or use of any of claims 39-41, wherein the excessive intracellular cyclic AMP signalling is caused by:
a. excessive hormone levels produced by an adenoma.
b. a gain-of-function gene mutation in a G-protein coupled receptor (GPCR);
c. an activating mutation in the GNAS1 gene, which encodes the α-subunit of the G-protein Gs; or
d. a bacterial toxin.

43. The compound or pharmaceutically acceptable salt or derivative or pharmaceutical composition for use of any of claim 1-16 or 36, 37, 38 or 42, or the method or use of any of claims 40-43, wherein the disease is cancer, optionally wherein the cancer is prostate cancer.

44. The compound or pharmaceutically acceptable salt or derivative or pharmaceutical composition for use of any of claim 1-16 or 36, 37, 38 or 42, or the method or use of any of claims 39-42, wherein the disease is:
a. pituitary adenoma, Cushing's disease, polycystic kidney disease or polycystic liver disease;
b. hyperthyroidism, Jansens's metaphyseal chondrodysplasia, hyperparathyroidism, or familial male-limited precocious puberty;
c. McCune-Albright syndrome;
d. cholera, whooping cough, anthrax, or tuberculosis;
e. HIV, AIDS, or Common Variable Immunodeficiency (CVID);
f. melanoma, pancreatic cancer, leukaemia, prostate cancer, adrenocortical tumours, testicular cancer, primary pigmented nodular adrenocortical diseases (PPNAD), or Carney Complex;
g. autosomal dominant polycystic kidney disease (ADPKD) or autosomal recessive polycystic kidney disease (ARPKD); or
h. maturity onset diabetes of young type 5 (MODY5); or
i. cardiac hypertrophy.

45. The compound or pharmaceutically acceptable salt or derivative or pharmaceutical composition for use, method or use of claim 44, wherein the disease is:
a. autosomal dominant polycystic kidney disease (ADPKD) or autosomal recessive polycystic kidney disease (ARPKD); or
b. hyperparathyroidism.
